SEQUENCE LISTING (5 sequences)

SEQ ID NO: 1 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 10 aa, Artificial Sequence
  Synthetic
GGGGSGGGGS

SEQ ID NO: 4 - protein, 15 aa, Artificial Sequence
  Synthetic
GGGGSGGGGSGGGGS

SEQ ID NO: 5 - protein, 7 aa, Artificial Sequence
  Synthetic
ENLYFQG

(19) United States
(12) Patent Application Publication
(10) Pub. No.: US 2020/0158716 A1
(43) Pub. Date: May 21, 2020

(54) CELL ATLAS OF HEALTHY AND DISEASED BARRIER TISSUES

(71) Applicant: Massachusetts Institute of Technology, Cambridge, MA (US)

(72) Inventors: Alexander K. Shalek, Cambridge, MA (US); Jose Ordovas-Montanes, Cambridge, MA (US)

(21) Appl. No.: 16/631,898

(22) PCT Filed: Jul. 17, 2018
(86) PCT No.: PCT/US2018/042557

§ 371 Date: Jan. 17, 2020

Related U.S. Application Data
(60) Provisional application No. 62/533,639, filed on Jul. 17, 2017.
(60) Provisional application No. 62/585,534, filed on Nov. 13, 2017.
(60) Provisional application No. 62/690,304, filed on Jun. 26, 2018.

(51) Int. Cl. G01N 33/50; C12Q 1/6886

(52) U.S. Cl. CPC G01N 33/5032; C12Q 2600/158; C12Q 2600/106; C12Q 1/6886

(57) ABSTRACT

Embodiments disclosed herein provide a cell atlas of barrier tissues from healthy and diseased subject. The atlas was obtained by single cell sequencing of approximately 18,036 cells in a surgical data set and 18,704 cells from scrapings. The present invention discloses novel markers for cell types. Moreover, genes associated with disease, including type 2 inflammation are identified. The invention provides for diagnostic assays based on gene markers and cell composition, as well as therapeutic targets for controlling differentiation, proliferation, maintenance and/or function of the cell types disclosed herein. In addition, novel cell types and methods of quantitating, detecting and isolating the cell types are disclosed.

### CROSS-REFERENCE TO RELATED APPLICATIONS

This application claims the benefit of U.S. Provisional Application Nos. 62/533,639, filed Jul. 17, 2017, 62/585,534, filed Nov. 13, 2017 and 62/690,304, filed Jun. 26, 2018. The entire contents of the above-identified applications are hereby fully incorporated herein by reference.

### STATEMENT REGARDING FEDERALLY SPONSORED RESEARCH

This invention was made with government support under Grant Nos. OD020839, AI089992, CA217377, AI039671, AI118672, HG006193 and CA202820 awarded by the National Institutes of Health. The government has certain rights in the invention.

### TECHNICAL FIELD

The subject matter disclosed herein is generally directed to a cell atlas of barrier tissue cell types in healthy and disease states. The subject matter further relates to novel cell specific and disease specific markers and therapeutic targets.

### BACKGROUND

Upper respiratory inflammation can afflict individuals on both acute and chronic timescales. Chronic inflammation of the upper airway sinuses is clinically referred to as chronic rhinosinusitis (CRS). The main pathophysiological division within CRS is between individuals who develop an inflamed mucosa (CRSsNP), and those who develop structures known as nasal polyps (CRSwNP), which are outgrowths of cells from the normal mucosa and can completely obstruct nasal passages. Both types of CRS exhibit an inflammatory pattern that is consistent with exuberant Type 2 immunity (T2I). However, our understanding of the drivers of phenotypic divergence and polyp formation remain unclear.

### SUMMARY OF THE INVENTION

Applicants report the first single-cell transcriptomes for human respiratory epithelial cell subsets, immune cells, and parenchymal cells (18,036 total cells) from an allergic inflammatory disease and map key T2I mediators.

In certain embodiments, the cell type may be detected by measuring one or more markers for each cell type selected from Table 1-15.

In another aspect, the present invention comprises a method for detecting type 2 inflammation, including chronic type 2, inflammation in a barrier tissue, comprising detecting loss of cell type diversity, including increase basal cell composition, by detecting one or more markers from Table 1-15.

In certain embodiments, the cell type as defined by expression of the markers described herein may be obtained by sorting cells based on expression of one or more markers for each cell type according to Table 1-15. In certain example embodiments, the quantity of cells may be determined by cell specific markers and gene expression assigned to each cell. In another aspect, the present invention comprises an isolated barrier cell characterized by expression of one or more markers from Table 1-15.

In another aspect, the present invention provides methods for detecting or quantifying barrier cells in a biological sample of a subject, the method comprising detecting or quantifying in the biological sample barrier cells as defined in any embodiment herein. The barrier cells may be detected or quantified using one or more cell surface markers for a cell type selected from Table 1-15.

In another aspect, the present invention provides for a method of isolating a barrier cell from a biological sample of a subject, the method comprising isolating from the biological sample barrier cells as defined as defined in any embodiment herein. The barrier cell may be isolated using one or more surface markers for a cell type selected from Table 1-15.

In certain embodiments, the barrier may be isolated, detected or quantified using a technique selected from the group consisting of RT-PCR, RNA-seq, single cell RNA-seq, western blot, ELISA, flow cytometry, mass cytometry, fluorescence activated cell sorting, fluorescence microscopy, affinity separation, magnetic cell separation, microfluidic separation, and combinations thereof.

In another aspect, the present invention provides for a method of modulating the barrier cell composition comprising treating a subject with an agent capable of targeting a barrier cell and inducing it to differentiate.

In another aspect, the present invention provides for a method of modulating barrier cell proliferation, differentiation, maintenance and/or function, comprising: contacting a barrier cell or population of barrier cells with a barrier cell modulating agent in an amount sufficient to modify differentiation, maintenance, and/or function of the barrier cell or population of barrier cells as compared to differentiation, maintenance, and/or function of the barrier cell population or population of barrier cells in the absence of the barrier cell modulating agent.

In another aspect, the present invention provides for a method for modulating cellular interactions within cellular ensembles, comprising administering to a cellular ensemble a modulating agent in an amount sufficient to change or modify extracellular signaling from a first cell type such that a change in one or more cell states is induced in a second cell type. In certain embodiments, the cellular ensemble is a two dimensional (2D) or three dimensional (3D) in vitro or ex vivo culture, a tissue on a chip, an organoid, or in vivo cells within a defined tissue, tissue compartment, or signaling microenvironment.

In certain embodiments, the first cell type comprises one or more pathogenic cells and the second cell type comprises on or more host cell types. In certain embodiments, the first cell type comprises one or more diseased cell types and the second cell type comprises one or more healthy cell types. The one or more diseased cell types may be cancer cells. The first cell type may comprise one or more stem cell types and the second cell type comprises one or more immune cell types.

In certain embodiments, extracellular signaling is receptor-ligand mediated, cytokine/chemokine mediated, or metabolically mediated. In certain embodiments, the cellular ensemble comprises epithelial tissues. The first cell type may be an immune cell and the second cell type may be an epithelial stem cell. The epithelial stem cell may be a basal cell.

In certain embodiments, the modulating agent antagonizes IL-4/13 signaling in the immune cells such that the differentiation of basal cells is induced.

In another aspect, the present invention provides for a method of treating inflammatory disease in barrier tissues comprising administering to a subject in need thereof, an IL-4 and/or IL-13 modulating agent in an amount sufficient to induce basal cell differentiation. In certain embodiments, the modulating agent blocks IL-4 and/or IL-13 signaling. In certain embodiments, the modulating agent reduces or blocks induction of CTNNB 1/Wnt.

In certain embodiments, the method further comprises administration of a second modulating agent, wherein the second modulating agent targets one or more gene or gene products selected from the group consisting of: FAM3C and CTNNB1; or PHAX, LPP, PDE6A, MEFV, TVP23C, PLCXD1, PHACTR4, AP1S3, TRAF3IP2, PGAM5, SMYD4, UGGT1, DFFA, UBB, LOC284454, IFITM3, TUBB2A, IRF1, AXL, SPN, RNF41, ANGPTL4, TUBB2B, NMNAT1, TANGO2, HOOK3, S1PR2, HLA-H, LRTOMT, ZNF430, SCOC, ZNF793, CYP20A1, MDM2, KREMEN1, ZC3H12D, FBLIM1, LOC646214, PDDC1, HLA-E, KRT6C, HYDIN2, COX18, PPM1K, MBOAT1, LINC00294, ZNF526, P2RX5-TAX1BP3, GNE, NUDT19, KIF18B, PDE4C, LETM1, TLCD2, CHST6, METTL21A, IKZF3, POTEE, HLA-G, XPNPEP3, TMEM33, MOG, TSPYL1, RBMS2, TAPBP, ANKRD20A9P, STON2, CYP4V2, POTEM, OPA3, POLH, ZNF805, IFITM2, C17orf75, IRGQ, KCNQ1OT1, TAF8, UGDH-AS1, CCL5, NOL9, CHP1, ORAI2, CA5B, HLA-B, LYZ, TOR1AIP2, TRAPPC2, SGTB, ZNF264, XIAP, RAMP2-AS1, SCAI, ZBTB3, ZNF490, ORC4, DNAL1, FBXL18, CPT1A, TNFAIP8L1, LRRC57, RBM3, HLA-C, LOC148709, LYRM7, ATCAY, PRICKLE2-AS3, AGMAT, NF2, LOC100131257, ACTG1, RUNDC1, MAVS, RPL36A-HNRNPH2, PNPO, GDPD1, ILF3-AS1, LOC284023, PRR11, TMEM41B, ZBTB8A, TUBB4A, ZNF850, VHL, IVD, FOXK1, MDM4, CCDC142, TRPV1, UBC and SENP5; or TM4SF1, IL1B; or CD44, CTNNB1, POSTN and TNC; or CD44, ANXA2, YBX1, EDN1, ARPC2, VAMP8, RPS18, EHF, HAS3, MBNL2, MTRNR2L9, CPXM2, NOS2, HMGB3, TNC, IFITM3, TSPAN3, BIK, CXADR, LGALS7, SERPINFI, TPM4, SERINC5, IGHG4, SERPINE2, NTRK2, PAPSS2, SDPR, ANXA3, RPL21, POSTN, CXCL2, MTRNR2L1, NDRG1, TPI1, SERPINB4, WIPI1, AL353147.1, CKB, GCLM, LRRC17, FAM110C, RPL18A, CAV1, VAMPS, IGHA1, VSNL1, KCNE3, SPARCL1, ARID5B, ARL4C, MTRNR2L3, CXCL1, IGHG1, CNN3, CST1, MYO10, IGHG3, AAMDC, HSPH1, ENO1, VCL, SYNGR2, LAMTOR4, CCL26, TSLP, SERPINB2, TMEM123, TGIF1, IGHM, RPL7, CMYA5, SH3BGRL3, LOXL4, MAP3K8, ANO1, PGAM1, STOM, MYL9, PLS1, CTNNAL1, EFNA1, MTRNR2L8, RPL13A, PAWR, RPL41, LDLR, ITGB1, TCEB2, SERPINB10, NEDD9, SERPINB13, LDHA, DSE, DDX58, GPC3, SERF2, KLHL5, MRPS6, ADAM9, CDCP1, EGLN3, SMARCADI, CTNNB1, MIF, PFN1, CTD-2228K2.5, CDH26, IGHA2, ST6GAL1, PLEKHA1, RPS25, GALNT7, CTD-2090113.1, SNRPG, RPL17, SRGN, ACTN1, MTRNR2L13, MORF4L1, RPSAP58, AREG, PLEKHA5, TMSB4X, IFITM1, SLC5A3, GEM, REXO2, KCNJ16, RPS4Y1, MMP10, NRG1, TBC1D9, LBH, FBXO32, TXNDC17, PHLDB2, SLC6A14, KRT6A, CP, TNFAIP3, CTD-2319112.1, SERTAD4-AS1, AMD1, FOSL1, LRRFIP1, IL6, UBBP4, BTG3, SLC2A1, IGJ, TMA7, SSR4, FGFBP1, HSD17B13, CCDC51, SMOC2, SHISA5, CTSC and XPR1; or HLA-F, SOX4, MYL12B, BST2, SHC1, INHBA, IFI6, SQSTM1, LYPLA1, RAP1B, MT1L, ANXA8L1, PLTP, NBPF10, MGST3, PLEKHB2, ICAM1, PHLDA1, PPP1CB, PAFAH1B2, SLC3A2, ANXA8L2, ISG20, GNG12, TNFRSF10B, GRN, DSG3, FN1, IFI27, C6orf62, SERPINE1, BNIP3, HLA-L, AKIRIN1, SYPL1, EFNA1, CDKN1A, TUBA1A, HLA-A, CCND2, CACUL1, RPS26, B2M, FKBP9L, AHNAK2, IFITM1, TFPI2, RPS4Y1, SNAI2, KRT6A, SRP9, CTGF and PGK1; or CDH3, MYO1B, HAS3, EHF, TNC, DKK3, SERPINB4, FABP5, TNFSF10, PTHLH, TBL1XR1, GJB2, CAV1, THBS1, TNS4, SHISA9, LOC100132247, KRT18, LITAF, HSPH1, CCL26, CSF3, KRT8, SPHK1, HK1, CDH6, SERPINB3, TP63, KRT23 and CTSC; or ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4 and EGR1; or ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4, EGR1, GLUL, ANXA1, EGR3, JUN, ID1, LMNA, JUNB, KRT17, HSPA1B, PPP1R15A, TSC22D1, SERPINE1, ETS2, DNAJB1, MTSS1L, ERRFI1, KRT5, TIPARP, LGALS3, SERTAD1, TSC22D3, PTP4A1, BPIFB1, MAFF, MUC5AC, FOSB, PMAIP1, HSPA8, ZFP36L2, IER2, NFKBIA, CDKN1A, BRD2, RPS16, CAPS, EZR, FAM107B, KRT19, F3, KLF4, RHOB, FOSL2, GPRC5A, SOCS3, TRIB, MIR22HG, HSP90AA1, PRDX1, WEE1, FTL, SLC25A25, VMP1, DDIT3, RND3, ATP5G2, TACSTD2, HBEGF, CD55, EMP1, MCL1, CEBPD, FKBP5, YPEL5, ACTG1, C12orf57, KAL1, RN7SK, NR1D2, FXYD3, KLF5, FOS, ANKRD37, MIDN, PER2, STARD7, WFDC2, DUSP6, HSPB1, SEPW1, MYC, CXCL17, KLF9, SYT8, GPX4, MIR24-2, EPS8, AHNAK, JUP, ADH7, S100P, POLR2A, PPP1R10, GCNT1, GAPDH, IER3, HOOK1, HN1L, TOMM20, SCGB1A1, GSR, SERPINB5, S100A6, PEBP1, CDC42EP4, AQP3, ADRB2, DDIT4, PER1, ADM, IRF1, PRDX6, RPS14, TPT1, PLXNB1, BHLHE40, SQSTM1, SCP2, KRT15, SGK1, ACTB, NABP1, IGFBP3, PPP1R15B, SOD2, SOX9, CTSH, MXD1, THBS1, CIB1, RAP2B, NEDD4L, RAB34, PRDX5, ANAPC11, RAC1, ARF1, HS3ST1, MAOA, HLA-E, EID1, BAG5, SERPINH1, CITED2, AJUBA, CTSK, PAFAH1B2, GNB2L1, LMO7, PDPN, UBE3A, HNRNPAB, GSTP1, TM4SF1, PIM3, DLX5, C9orf3, HPGD, ANXA11, UBC, PLP2, EGR2, S100A10, LAMA5, ALOX15, MUC16, RPL4, TNS4, TUBB, RHOC, CYP2S1, PPAP2C, GPX1, QARS, CSNK1D, HINT1, PDLIM1, BLVRB, PSMB6, TSPO, CDCA7L, HNRNPF, RPS4X, EPB41L4A-AS1, IRF6, FSTL1, FTH1, UBL5, ADI1, PHLDA1, IFITM2, VAPA, PPP1CB, EIF4A3, UBB, SELM, NFKBIZ, UBR5, PTPN14, RAN, PPP2CA, CKS2, ELF3, EIF4A1, CSTB, TSPYL2, PLEC, UGDH, IBTK, TUBA1C, HLA-A, PHYHDI, RNA28S5, UBA52, RPL30, FLOT1, CHST9, TMEM14B, EIF1, PTHLH, STAT6, LRRC8A, MTRNR2L2, VPS37B, HN1, ELK4, NBN, MID1, RAB2A, CCND2, PSMB1, KDSR, SIK1, KDM2A, CDC42SE2, SLC35F5, CLTA, SF3B14, TUBAIB, C6orf48, SRSF3, RPS12, ARHGAP18, C16orf72, ALDOA, MRFAP1, LYPLAL1, JUND, TRAK1, MINK1, RPL11, HMGN2, LNX2, CFL1, TAGLN2, SPRY2, HLA-C, SDC4, EEF2, PCBP1, PER3, RPLP1, RPL8, REV1, S100A2, NDUFV1, EIF3K, MYADM, DDX3Y, KRT8, SLPI, CHD4, SEMA5A, TOR1AIP2, CHD2, GSN, GDI2, HLA-B, FLRT3, PERP, DNAJA1, OBSCN, TNFRSF1A, PSMB3, KLF6, WDR26, TFCP2L1, ZCRB1, LITAF, SNRNP70, EIF3I, SPINT1, FLOT2, SLC3A2, UGT2A2, ID2, TMEM59, HMGA1, CTNND1, FKBP9, TUBB4B, POLR2G, SLC9A3R1, SNHG5, HEBP2, RNF181, BCAM, SRI, ARHGDIA, HERPUD1, KLF10, MAT2A, LEPROT, SPCS1, SORL1, CCNL1, CYB5R3, USP22, MKNK2, SEC14L1, SEC61B, BTF3, FZD6, DAZAP2, SFN, MGST1, LAMB3, TNFRSF12A, OAT, CD99, CAV2, FLNA, STXBP3, RIPK4, SRSF7, EIF3D, TNPO1, MYL12B, GCLC, METTL7A, RPL28, F2R, RNA18S5, TMEM261, SOD1, YBX3, Cllorf31, MYL6, ELOVL5, ATOX1, RPLP2, FBXL5, MAP3K13, MDK, TOB1, DLL1, HSP90AB1, SNCA, LAMA3, CSNK1A1, ATP1B3, RPS2, EIF4H, UBE2D3, CEBPB, TGFBR2, RPS3, GADD45B, GAS5, ZNHIT1, DDR1, JAG2, WNT4, NDFIP1, APH1A, OAZ1, HDAC7, ATP1A1, FAU, PPL, RAB11A, NCOA4, ATP5O, SF1, UQCRQ, NONO, FBXL3, ID3, DHCR24, IER5, OPTN, TMSB10, PPP1CA, ARPC5, RAB1A, TSKU, RPL19, BTG1, NACA, RAI14, CAPN1, 10-Sep, CDKN2AIP, PHB2, DDOST, S100A16, BLCAP, CSDE1, EDF1, CDK5RAP2, PRKAR1A, CNN2, FGFR3, ALDH3A2, TMX4, RPL7A, RPS27, C14orf166, RPL18, C7orf55-LUC7L2, GNG5, GHITM, IVNS1ABP, H3F3B, RBM3, RPL27, SMIM15, ARFGAP3, PPP4R1, NDUFB4, SSBP1, RPS21, DDB1, ALAS1, SERINC3, TMBIM6, AQP5, RPS5, EIF3F, 7-Mar, SDF4, RPL15, CDC42SE1, LGALS8, PAK1, NAMPT, EGFR, NDUFS1, GPR155, RAD23B, CD9, LIMK2, DLK2, CBR1, PPP2R5C, PRRC2C, RPS11, SH3GLB1, HNRNPAO, NUMA1, FGFR2, GABARAPL2, ETV3, H3F3C, SSH1, ATF4, SH3BGRL, ABHD2, ZFYVE21, RPS24, SYNCRIP, DDX17, RPS8, AFG3L2, RPL26, HNRNPA3, VPS26A, CHD8, RPS19, NCL, PDCD10, DST, POMP, APP, CLU, SRP9, SKP1, RPL41, RPL39, UQCRB, MTUS1, IFT81, USP8, RPL6, RNF19A, ADAM28, LSM3, ASPH, GOLGB1, HNRNPH1, CP, TPM3, EEF1D, PDIA4, PTMA, CMYA5, PGAP1, MPHOSPH6, MTRNR2L8, MALAT1, VMO1, ITGAV, PDIA3, TLK1, RPL21, MTRNR2L10, CXADR, SPCS2, HSPA5, POSTN, LARS, MORF4L1, ATPSI, TNC, AKAP9, TMSB4X, BPTF, SESN3, SYNE2, TTC3, C1R, RPL10, MT-ND6, SERPINB4, PHPT1, RPL34, IGFBP2, MTRNR2L1, SLC38A1, SMC6, ENAH, SON, HLA-DRA, YWHAE, HSP90B1, MTRNR2L7, MTRNR2L5, MTRNR2L11, MT-ND2, MTRNR2L3, CTC-338M12.5, MTRNR2L13, MMP10, MT-RNR1, IGHA1, IGJ, MT-ATP8, MT-ND1, MT-ND5, MT-RNR2, MT-C02, MT-CYB, MT-ATP6, MT-ND4L, MT-C03, IGKC, MT-ND4, and MT-CO1; or SERPINB3, SERPINB4, FABP5, CTSC, GJB2, SPHK1, MBOAT1, ORAI2, KCNQ1OT1, PDDC1, CHP1, CCL5, CYP20A1, IGFBP7, LRTOMT, TOR1AIP2, XPNPEP3, LOXL4, TRAPPC2, ZNF490, GNE, LOC100131257, ZBTB8A, TAPBP, TNFAIP8L1, UGDH-AS1, CCL26, CDH6, IRF1, LOC646214, TNS4, PRICKLE2-AS3, KRT16, PRR11, RBMS2, LYZ, FAM3C, NOL9, POLH, DFFA, PPM1K, UBB, METTL21A, CAV1, IKZF3, PDE4C, SHISA9, AP1S3, HAS3, IRGQ, XIAP, FBLIM1, PHACTR4, SPN, C17orf75, HLA-B, HLA-C, KRT23, NMNAT1, HOOK3, KRT75, THBS1, CCDC142, HYDIN2, LETM1, CAV2, RUNDC1, TLCD2, PLCXD1, HLA-E, UGGT1, UBC, ZNF793, LOC643406, TNC, PNPO, FBXL18, EHF, ZNF264, SMYD4, LEPREL4, RPL36A-HNRNPH2, LRRC57, ANKRD20A9P, VHL, CDH3, ZNF850, MOG, TAF8, RNF41, GDPD1, KRT8, COTL1, PNPT1, ORC4, TRAF3IP2, NUDT19, RNF125, IVD, ODF2L, BTG1, OR7D2, MEFV, MDM4, CTNNB1, OPA3, LINC00294, ZNF526, CRX, CYP4V2, GLTP, KIF18B, SPREDI, MYO1B, ILF3-AS1, TANGO2, TMEM41B, SCAI, LYRM7, TRPV1, CYCS, TUBB2A, CA5B, ATCAY, ASTN2, SLC16A3, POTEE, PDE6A, P2RX5-TAX1BP3, TUBA4A, DDX51, CPT1A, TMEM212, KREMEN1, GPR82, S1PR2, SENP5, PHAX, TUBB4B, ZBTB3, TVP23C, PXMP4, WDR92, OPHN1, AP4S1, LOC284023, L2HGDH, TBC1D24, MTFMT, SGTB, PGAM5, SLFNL1-AS1, IFNLR1, GJC1, RAMP2-AS1, FKBP14, MDM2, KRT6C, AGMAT, CHST6, NWD1, MAVS, AKIP1, FOXK1, METTL2A, FDPSL2A, HLA-G, TFDP2, VSIG1, ZNF483, PTCHD4, SLC6A4, ZNF805, DNAL1, LOC284454, CD3EAP, PIGX, C10orf32, COX18, HLA-H, CPM, FBXO27, PALLD, LPP, NXN, NMT2, NPIPL3, PARD6B, VPS53, FUT1, GREB1, C21orf62, TINAGLI, WDR55, ZC3H12D, DTX3L, NF2, GATAD1, TRPM7, QPCTL, CFLAR, AXL, ALDH1A3, ARPP19, ZNF814, PSTPIP2, LOC90834, MREG, MPPE1, ZFP14, NT5DC3, SLC35F6, MAP1LC3C, PER2, UTP11L, TMEM136, KIN, NPHS1, GK5, PTHLH, LOH12CR2, GNL3L, MXRA7, TUBB4A, TSPYL1, POTEM, FBXL20, TBL1XR1, HP1BP3, TMEM33, PTRF, MYLK3, IFITM3, CABP4, LINC00338, ICA1L, MRI1, EMX2OS, IRAK4, IBA57, LOC728558, POU5F1, ZKSCAN3, PCDH11X, STON2, SLC50A1, LOC613037, AKAP5, ZNF430, ZNF587, EMP2, SLC5A5, STK4, RAC1, ARGFX, MCTS1, MAPK13, ULK2, CYB5R3, PEX13, LOC100132247, LOC100506190, MS4A10, PPP1CC, LIMS1, DCAF10, TBXA2R, ANAPC16, CD84, LOC284950, ACTG1, DLEU2, TRIM45, CCBE1, MED18, ME2, SLC28A2, ZNF738, DAND5, TTC39C, WHAMM, TMEM120B, FLVCR1, SMU1, CEP104, GTF2H3, FXN, IFITM2, RPS2, NME1-NME2, LOC727896, ZYG11B, LOC100507173, MTCH1, ATXN3, NLRP12, TUBB2B, ENPP1, MRPS16, SHOX, SLC35E3, GCLM, EDARADD, ESYT2, C12orf65, CNNM3, BRIP1, GOLGA3, FAM227A, TM7SF3, FCAR, DBT, CACNG8, ANPEP, LOC100287792, SLC12A6, LITAF, INMT, GTF2H2C, SIX4, CYP51A1, ALG1, SPIB, LRPAP1, DKK3, PRRG4, SNHG16, ARSA, RABL5, PACS2, DNAJC22, RELL1, LOC100289019, SCAMP4, KRT18, ZXDC, MOB3A, EXPH5, PCBD2, LOC286437, ANGPTL4, ABCC9, SNIP1, AFMID, C lorf58, GEMIN8, LOC148709, PTGIS, ZNF785, RNF168, SHROOM1, ZSCAN29, CARD8, FAM122C, FAM73A, SDE2, HSPH1, NLRC3, HAUS3, IAPP, SPAST, PNMA2, LRRC58, LOC100506746, GFOD2, UBE2Q2P1, APOL1, LRP10, CORO2A, ZSCAN22, TUBB6, MIR143HG, HNF1A-AS1, ZNF865, SPATS2, SLC25A32, CCL22, LOC100505876, ADAMTS4, RPS6KA3, TRIM16L, PDLIM5, MPL, ENTPD4, ADAT1, SAR1B, UCKL1-AS1, CYP1A2, METTL2B, ASB6, CEACAM22P, ABL2, LRRC27, ENAH, TP63, ZFAND5, LOC100506688, AK3, PPIEL, OCLN, WAC, SPATA5, TNFSF10, SCOC, LRRN4CL, BHMT2, PTAFR, GSTM3, FKBP5, TNFAIP8L2-SCNM1, PDK3, ZNF714, CXorf56, TRMT2B, CBFA2T2, SLC35E2, MTDH, EEF2K, HAUS2, TPMT, CEP68, SLC4A8, TSIX, CXADR, KLRD1, TMEM165, IDS, SS18, EFNB1, APOBEC3F, ADRA1A, SLC25A15, CLCC1, CBX5, STYX, RBBP5, GNG4, RBM34, ZNF829, RBM3, JAK3, FZD3, ZYG11A, ARHGAP1, PLEKHG2, NUP43, CXorf38, TRIM58, ZNF818P, SKA1, MTPAP, Clorf174, MFSD11, MAPKIIP1L, GA™-AS1, DESI1, IVNS1ABP, GLT25D1, C4orf19, GLG1, RFT1, ZNF626, LPIN3, CSF3, GPR155, SSR1, FCF1, ZNF737, NDUFV3, ATP1A1, EIF2S3, ATP6V1G1, TADA3, CLSPN, TBC1D15, RAB27A, HK1, RTCA, ARNTL2, KCNA7, SMIM12 and ZSWIM1; or IFI27, HLA-A, HLA-B, HLA-C, HLA-H, IFITM3, RPS26, ICAM1, IFITM1, IFI6, UBC, SERPINE1, TFPI2, EFNA1, TUBB2A, HLA-E, HLA-G, AHNAK2, TUBA1A, TUBB2B, POTEE, B2M, FN1, MBOAT1, TAPBP, KRT6C, RPS4Y1, TUBB4A, ORAI2, TOR1AIP2, SQSTM1, MT1L, RPL36A-HNRNPH2, TSPYL1, IFITM2, PDDC1, UBB, CHP1, CCL5, LYPLA1, NBPF10, ZBTB8A, PPP1CB, KCNQ1OT1, CYP20A1, ZNF490, IL1B, ANXA8L1, XPNPEP3, CTNNB1, LRTOMT, SHC1, STON2, LYZ, UGDH-AS1, POTEM, MDM2, NOL9, HLA-F, IKZF3, TNFAIP8L1, PPM1K, SLC3A2, AP1S3, PRICKLE2-AS3, DFFA, POLH, IRF1, PAFAH1B2, PLEKHB2, XIAP, UGGT1, CTGF, LOC100131257, TRAPPC2, PDE6A, FBLIM1, PDE4C, PRR11, PNPO, C17orf75, SRP9, RBMS2, CCDC142, AXL, ZNF793, KRT6A, TMEM41B, RAP1B, GNE, RBM3, FOXK1, INHBA, NMNAT1, IRGQ, TLCD2, BST2, NF2, TRAF3IP2, CA5B, SENP5, NUDT19, LINC00294, HOOK3, CYP4V2, LOC646214, CACUL1, PHACTR4, VHL, ZNF526, ZNF264, RUNDC1, HYDIN2, FBXL18, SPN, GDPD1, PHAX, TAF8, LRRC57, METTL21A, IVD, MGST3, SMYD4, ANXA8L2, PLTP, TVP23C, TM4SF1, ZBTB3, FKBP9L, MDM4, C6orf62, ILF3-AS1, TRPV1, LETM1, CHST6, TNFRSF10B, S1PR2, KIF18B, CPT1A, MOG, RNF41, SOX4, PLCXD1, PGAM5, LPP, GNG12, MEFV, ZNF850, OPA3, SCAI, TMEM33, LYRM7, SNAI2, SCOC, DSG3, LOC148709, SGTB, GRN, ATCAY, SYPL1, KREMEN1, BNIP3, ANGPTL4, RAMP2-AS1, MAVS, ZC3H12D, P2RX5-TAX1BP3, CDKN1A, ISG20, ANKRD20A9P, ZNF805, LOC284454, HLA-L, AGMAT, AKIRIN1, MYL12B, TANGO2, ACTG1, LOC284023, COX18, ORC4, PHLDA1, DNAL1, FAM3C, PGK1, ZNF430 and CCND2.

In certain embodiments, the subject suffers from a chronic human inflammatory disease. The chronic human inflammatory disease may be characterized by basal cell hyperplasia. The chronic human inflammatory disease may comprise a Type 2 immunity response. The chronic human inflammatory disease may be chronic rhinosinusitis.

In certain embodiments, the subject in need thereof is selected based on the presence of an IL4/13 signature in on or more epithelia cell types. In certain embodiments, the IL4/IL13 signature comprises one or more genes selected from the group consisting of: FAM3C and CTNNB1; or PHAX, LPP, PDE6A, MEFV, TVP23C, PLCXD1, PHACTR4, AP1S3, TRAF3IP2, PGAM5, SMYD4, UGGT1, DFFA, UBB, LOC284454, IFITM3, TUBB2A, IRF1, AXL, SPN, RNF41, ANGPTL4, TUBB2B, NMNAT1, TANGO2, HOOK3, S1PR2, HLA-H, LRTOMT, ZNF430, SCOC, ZNF793, CYP20A1, MDM2, KREMEN1, ZC3H12D, FBLIM1, LOC646214, PDDC1, HLA-E, KRT6C, HYDIN2, COX18, PPM1K, MBOAT1, LINC00294, ZNF526, P2RX5-TAX1BP3, GNE, NUDT19, KIF18B, PDE4C, LETM1, TLCD2, CHST6, METTL21A, IKZF3, POTEE, HLA-G, XPNPEP3, TMEM33, MOG, TSPYL1, RBMS2, TAPBP, ANKRD20A9P, STON2, CYP4V2, POTEM, OPA3, POLH, ZNF805, IFITM2, C17orf75, IRGQ, KCNQ1OT1, TAF8, UGDH-AS1, CCL5, NOL9, CHP1, ORAI2, CA5B, HLA-B, LYZ, TOR1AIP2, TRAPPC2, SGTB, ZNF264, XIAP, RAMP2-AS1, SCAI, ZBTB3, ZNF490, ORC4, DNAL1, FBXL18, CPT1A, TNFAIP8L1, LRRC57, RBM3, HLA-C, LOC148709, LYRM7, ATCAY, PRICKLE2-AS3, AGMAT, NF2, LOC100131257, ACTG1, RUNDC1, MAVS, RPL36A-HNRNPH2, PNPO, GDPD1, ILF3-AS1, LOC284023, PRR11, TMEM41B, ZBTB8A, TUBB4A, ZNF850, VHL, IVD, FOXK1, MDM4, CCDC142, TRPV1, UBC and SENP5; or TM4SF1, IL1B; or CD44, CTNNB1, POSTN and TNC; or CD44, ANXA2, YBX1, EDN1, ARPC2, VAMP8, RPS18, EHF, HAS3, MBNL2, MTRNR2L9, CPXM2, NOS2, HMGB3, TNC, IFITM3, TSPAN3, BIK, CXADR, LGALS7, SERPINFI, TPM4, SERINC5, IGHG4, SERPINE2, NTRK2, PAPSS2, SDPR, ANXA3, RPL21, POSTN, CXCL2, MTRNR2L1, NDRG1, TPI1, SERPINB4, WIPI1, AL353147.1, CKB, GCLM, LRRC17, FAM110C, RPL18A, CAV1, VAMPS, IGHA1, VSNL1, KCNE3, SPARCL1, ARID5B, ARL4C, MTRNR2L3, CXCL1, IGHG1, CNN3, CST1, MYO10, IGHG3, AAMDC, HSPH1, ENO1, VCL, SYNGR2, LAMTOR4, CCL26, TSLP, SERPINB2, TMEM123, TGIF1, IGHM, RPL7, CMYA5, SH3BGRL3, LOXL4, MAP3K8, ANO1, PGAM1, STOM, MYL9, PLS1, CTNNAL1, EFNA1, MTRNR2L8, RPL13A, PAWR, RPL41, LDLR, ITGB1, TCEB2, SERPINB10, NEDD9, SERPINB13, LDHA, DSE, DDX58, GPC3, SERF2, KLHL5, MRPS6, ADAM9, CDCP1, EGLN3, SMARCADI, CTNNB1, MIF, PFN1, CTD-2228K2.5, CDH26, IGHA2, ST6GAL1, PLEKHA1, RPS25, GALNT7, CTD-2090I13.1, SNRPG, RPL17, SRGN, ACTN1, MTRNR2L13, MORF4L1, RPSAP58, AREG, PLEKHA5, TMSB4X, IFITM1, SLC5A3, GEM, REXO2, KCNJ16, RPS4Y1, MMP10, NRG1, TBC1D9, LBH, FBXO32, TXNDC17, PHLDB2, SLC6A14, KRT6A, CP, TNFAIP3, CTD-2319112.1, SERTAD4-AS1, AMD1, FOSL1, LRRFIP1, IL6, UBBP4, BTG3, SLC2A1, IGJ, TMA7, SSR4, FGFBP1, HSD17B13, CCDC51, SMOC2, SHISA5, CTSC and XPR1; or HLA-F, SOX4, MYL12B, BST2, SHC1, INHBA, IFI6, SQSTM1, LYPLA1, RAP1B, MT1L, ANXA8L1, PLTP, NBPF10, MGST3, PLEKHB2, ICAM1, PHLDA1, PPP1CB, PAFAH1B2, SLC3A2, ANXA8L2, ISG20, GNG12, TNFRSF10B, GRN, DSG3, FN1, IFI27, C6orf62, SERPINE1, BNIP3, HLA-L, AKIRIN1, SYPL1, EFNA1, CDKN1A, TUBA1A, HLA-A, CCND2, CACUL1, RPS26, B2M, FKBP9L, AHNAK2, IFITM1, TFPI2, RPS4Y1, SNAI2, KRT6A, SRP9, CTGF and PGK1; or CDH3, MYO1B, HAS3, EHF, TNC, DKK3, SERPINB4, FABP5, TNFSF10, PTHLH, TBL1XR1, GJB2, CAV1, THBS1, TNS4, SHISA9, LOC100132247, KRT18, LITAF, HSPH1, CCL26, CSF3, KRT8, SPHK1, HK1, CDH6, SERPINB3, TP63, KRT23 and CTSC; or ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4 and EGR1; or ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4, EGR1, GLUL, ANXA1, EGR3, JUN, ID1, LMNA, JUNB, KRT17, HSPA1B, PPP1R15A, TSC22D1, SERPINE1, ETS2, DNAJB1, MTSS1L, ERRFI1, KRT5, TIPARP, LGALS3, SERTAD1, TSC22D3, PTP4A1, BPIFB1, MAFF, MUC5AC, FOSB, PMAIP1, HSPA8, ZFP36L2, IER2, NFKBIA, CDKN1A, BRD2, RPS16, CAPS, EZR, FAM107B, KRT19, F3, KLF4, RHOB, FOSL2, GPRC5A, SOCS3, TRIB1, MIR22HG, HSP90AA1, PRDX1, WEE1, FTL, SLC25A25, VMP1, DDIT3, RND3, ATP5G2, TACSTD2, HBEGF, CD55, EMP1, MCL1, CEBPD, FKBP5, YPEL5, ACTG1, C12orf57, KAL1, RN7SK, NR1D2, FXYD3, KLF5, FOS, ANKRD37, MIDN, PER2, STARD7, WFDC2, DUSP6, HSPB1, SEPW1, MYC, CXCL17, KLF9, SYT8, GPX4, MIR24-2, EPS8, AHNAK, JUP, ADH7, S100P, POLR2A, PPP1R10, GCNT1, GAPDH, IER3, HOOK1, HN1L, TOMM20, SCGB1A1, GSR, SERPINB5, S100A6, PEBP1, CDC42EP4, AQP3, ADRB2, DDIT4, PER1, ADM, IRF1, PRDX6, RPS14, TPT1, PLXNB1, BHLHE40, SQSTM1, SCP2, KRT15, SGK1, ACTB, NABP1, IGFBP3, PPP1R15B, SOD2, SOX9, CTSH, MXD1, THBS1, CIB1, RAP2B, NEDD4L, RAB34, PRDX5, ANAPC11, RAC1, ARF1, HS3ST1, MAOA, HLA-E, EID1, BAG5, SERPINH1, CITED2, AJUBA, CTSK, PAFAH1B2, GNB2L1, LMO7, PDPN, UBE3A, HNRNPAB, GSTP1, TM4SF1, PIM3, DLX5, C9orf3, HPGD, ANXA11, UBC, PLP2, EGR2, S100A10, LAMA5, ALOX15, MUC16, RPL4, TNS4, TUBB, RHOC, CYP2S1, PPAP2C, GPX1, QARS, CSNK1D, HINT1, PDLIM1, BLVRB, PSMB6, TSPO, CDCA7L, HNRNPF, RPS4X, EPB41L4A-AS1, IRF6, FSTL1, FTH1, UBL5, ADI1, PHLDA1, IFITM2, VAPA, PPP1CB, EIF4A3, UBB, SELM, NFKBIZ, UBR5, PTPN14, RAN, PPP2CA, CKS2, ELF3, EIF4A1, CSTB, TSPYL2, PLEC, UGDH, IBTK, TUBA1C, HLA-A, PHYHDI, RNA28S5, UBA52, RPL30, FLOT1, CHST9, TMEM14B, EIF1, PTHLH, STAT6, LRRC8A, MTRNR2L2, VPS37B, HN1, ELK4, NBN, MID1, RAB2A, CCND2, PSMB1, KDSR, SIK1, KDM2A, CDC42SE2, SLC35F5, CLTA, SF3B14, TUBAIB, C6orf48, SRSF3, RPS12, ARHGAP18, C16orf72, ALDOA, MRFAP1, LYPLAL1, JUND, TRAK1, MINK1, RPL11, HMGN2, LNX2, CFL1, TAGLN2, SPRY2, HLA-C, SDC4, EEF2, PCBP1, PER3, RPLP1, RPL8, REV1, S100A2, NDUFV1, EIF3K, MYADM, DDX3Y, KRT8, SLPI, CHD4, SEMA5A, TOR1AIP2, CHD2, GSN, GDI2, HLA-B, FLRT3, PERP, DNAJA1, OBSCN, TNFRSF1A, PSMB3, KLF6, WDR26, TFCP2L1, ZCRB1, LITAF, SNRNP70, EIF3I, SPINT1, FLOT2, SLC3A2, UGT2A2, ID2, TMEM59, HMGA1, CTNND1, FKBP9, TUBB4B, POLR2G, SLC9A3R1, SNHG5, HEBP2, RNF181, BCAM, SRI, ARHGDIA, HERPUD1, KLF10, MAT2A, LEPROT, SPCS1, SORL1, CCNL1, CYB5R3, USP22, MKNK2, SEC14L1, SEC61B, BTF3, FZD6, DAZAP2, SFN, MGST1, LAMB3, TNFRSF12A, OAT, CD99, CAV2, FLNA, STXBP3, RIPK4, SRSF7, EIF3D, TNPO1, MYL12B, GCLC, METTL7A, RPL28, F2R, RNA18S5, TMEM261, SOD1, YBX3, Cllorf31, MYL6, ELOVL5, ATOX1, RPLP2, FBXL5, MAP3K13, MDK, TOB1, DLL1, HSP90AB1, SNCA, LAMA3, CSNK1A1, ATP1B3, RPS2, EIF4H, UBE2D3, CEBPB, TGFBR2, RPS3, GADD45B, GAS5, ZNHIT1, DDR1, JAG2, WNT4, NDFIP1, APH1A, OAZ1, HDAC7, ATP1A1, FAU, PPL, RAB11A, NCOA4, ATP5O, SF1, UQCRQ, NONO, FBXL3, ID3, DHCR24, IER5, OPTN, TMSB10, PPP1CA, ARPC5, RAB1A, TSKU, RPL19, BTG1, NACA, RAI14, CAPN1, 10-Sep, CDKN2AIP, PHB2, DDOST, S100A16, BLCAP, CSDE1, EDF1, CDK5RAP2, PRKAR1A, CNN2, FGFR3, ALDH3A2, TMX4, RPL7A, RPS27, C14orf166, RPL18, C7orf55-LUC7L2, GNG5, GHITM, IVNS1ABP, H3F3B, RBM3, RPL27, SMIM15, ARFGAP3, PPP4R1, NDUFB4, SSBP1, RPS21, DDB1, ALAS1, SERINC3, TMBIM6, AQP5, RPS5, EIF3F, 7-Mar, SDF4, RPL15, CDC42SE1, LGALS8, PAK1, NAMPT, EGFR, NDUFS1, GPR155, RAD23B, CD9, LIMK2, DLK2, CBR1, PPP2R5C, PRRC2C, RPS11, SH3GLB1, HNRNPAO, NUMA1, FGFR2, GABARAPL2, ETV3, H3F3C, SSH1, ATF4, SH3BGRL, ABHD2, ZFYVE21, RPS24, SYNCRIP, DDX17, RPS8, AFG3L2, RPL26, HNRNPA3, VPS26A, CHD8, RPS19, NCL, PDCD10, DST, POMP, APP, CLU, SRP9, SKP1, RPL41, RPL39, UQCRB, MTUS1, IFT81, USP8, RPL6, RNF19A, ADAM28, LSM3, ASPH, GOLGB1, HNRNPH1, CP, TPM3, EEF1D, PDIA4, PTMA, CMYA5, PGAP1, MPHOSPH6, MTRNR2L8, MALAT1, VMO1, ITGAV, PDIA3, TLK1, RPL21, MTRNR2L10, CXADR, SPCS2, HSPA5, POSTN, LARS, MORF4L1, ATP5I, TNC, AKAP9, TMSB4X, BPTF, SESN3, SYNE2, TTC3, C1R, RPL10, MT-ND6, SERPINB4, PHPT1, RPL34, IGFBP2, MTRNR2L1, SLC38A1, SMC6, ENAH, SON, HLA-DRA, YWHAE, HSP90B1, MTRNR2L7, MTRNR2L5, MTRNR2L11, MT-ND2, MTRNR2L3, CTC-338M12.5, MTRNR2L13, MMP10, MT-RNR1, IGHA1, IGJ, MT-ATP8, MT-ND1, MT-ND5, MT-RNR2, MT-CO2, MT-CYB, MT-ATP6, MT-ND4L, MT-CO3, IGKC, MT-ND4, and MT-CO1; or SERPINB3, SERPINB4, FABP5, CTSC, GJB2, SPHK1, MBOAT1, ORAI2, KCNQ1OT1, PDDC1, CHP1, CCL5, CYP20A1, IGFBP7, LRTOMT, TOR1AIP2, XPNPEP3, LOXL4, TRAPPC2, ZNF490, GNE, LOC100131257, ZBTB8A, TAPBP, TNFAIP8L1, UGDH-AS1, CCL26, CDH6, IRF1, LOC646214, TNS4, PRICKLE2-AS3, KRT16, PRR11, RBMS2, LYZ, FAM3C, NOL9, POLH, DFFA, PPM1K, UBB, METTL21A, CAV1, IKZF3, PDE4C, SHISA9, AP1S3, HAS3, IRGQ, XIAP, FBLIM1, PHACTR4, SPN, C17orf75, HLA-B, HLA-C, KRT23, NMNAT1, HOOK3, KRT75, THBS1, CCDC142, HYDIN2, LETM1, CAV2, RUNDC1, TLCD2, PLCXD1, HLA-E, UGGT1, UBC, ZNF793, LOC643406, TNC, PNPO, FBXL18, EHF, ZNF264, SMYD4, LEPREL4, RPL36A-HNRNPH2, LRRC57, ANKRD20A9P, VHL, CDH3, ZNF850, MOG, TAF8, RNF41, GDPD1, KRT8, COTL1, PNPT1, ORC4, TRAF3IP2, NUDT19, RNF125, IVD, ODF2L, BTG1, OR7D2, MEFV, MDM4, CTNNB1, OPA3, LINC00294, ZNF526, CRX, CYP4V2, GLTP, KIF18B, SPREDI, MYO1B, ILF3-AS1, TANGO2, TMEM41B, SCAI, LYRM7, TRPV1, CYCS, TUBB2A, CA5B, ATCAY, ASTN2, SLC16A3, POTEE, PDE6A, P2RX5-TAX1BP3, TUBA4A, DDX51, CPT1A, TMEM212, KREMEN1, GPR82, S1PR2, SENP5, PHAX, TUBB4B, ZBTB3, TVP23C, PXMP4, WDR92, OPHN1, AP4S1, LOC284023, L2HGDH, TBC1D24, MTFMT, SGTB, PGAM5, SLFNL1-AS1, IFNLR1, GJC1, RAMP2-AS1, FKBP14, MDM2, KRT6C, AGMAT, CHST6, NWD1, MAVS, AKIP1, FOXK1, METTL2A, FDPSL2A, HLA-G, TFDP2, VSIG1, ZNF483, PTCHD4, SLC6A4, ZNF805, DNAL1, LOC284454, CD3EAP, PIGX, C10orf32, COX18, HLA-H, CPM, FBXO27, PALLD, LPP, NXN, NMT2, NPIPL3, PARD6B, VPS53, FUT1, GREB1, C21orf62, TINAGL1, WDR55, ZC3H12D, DTX3L, NF2, GATADI, TRPM7, QPCTL, CFLAR, AXL, ALDH1A3, ARPP19, ZNF814, PSTPIP2, LOC90834, MREG, MPPE1, ZFP14, NT5DC3, SLC35F6, MAP1LC3C, PER2, UTP11L, TMEM136, KIN, NPHS1, GK5, PTHLH, LOH12CR2, GNL3L, MXRA7, TUBB4A, TSPYL1, POTEM, FBXL20, TBL1XR1, HP1BP3, TMEM33, PTRF, MYLK3, IFITM3, CABP4, LINC00338, ICA1L, MRI1, EMX2OS, IRAK4, IBA57, LOC728558, POU5F1, ZKSCAN3, PCDH11X, STON2, SLC50A1, LOC613037, AKAP5, ZNF430, ZNF587, EMP2, SLC5A5, STK4, RAC1, ARGFX, MCTS1, MAPK13, ULK2, CYB5R3, PEX13, LOC100132247, LOC100506190, MS4A10, PPPICC, LIMS1, DCAF10, TBXA2R, ANAPC16, CD84, LOC284950, ACTG1, DLEU2, TRIM45, CCBE1, MED18, ME2, SLC28A2, ZNF738, DAND5, TTC39C, WHAMM, TMEM120B, FLVCR1, SMU1, CEP104, GTF2H3, FXN, IFITM2, RPS2, NME1-NME2, LOC727896, ZYG11B, LOC100507173, MTCH1, ATXN3, NLRP12, TUBB2B, ENPP1, MRPS16, SHOX, SLC35E3, GCLM, EDARADD, ESYT2, C12orf65, CNNM3, BRIP1, GOLGA3, FAM227A, TM7SF3, FCAR, DBT, CACNG8, ANPEP, LOC100287792, SLC12A6, LITAF, INMT, GTF2H2C, SIX4, CYP51A1, ALG1, SPIB, LRPAP1, DKK3, PRRG4, SNHG16, ARSA, RABL5, PACS2, DNAJC22, RELL1, LOC100289019, SCAMP4, KRT18, ZXDC, MOB3A, EXPH5, PCBD2, LOC286437, ANGPTL4, ABCC9, SNIP1, AFMID, Cllorf58, GEMIN8, LOC148709, PTGIS, ZNF785, RNF168, SHROOM1, ZSCAN29, CARD8, FAM122C, FAM73A, SDE2, HSPH1, NLRC3, HAUS3, IAPP, SPAST, PNMA2, LRRC58, LOC100506746, GFOD2, UBE2Q2P1, APOL1, LRP10, CORO2A, ZSCAN22, TUBB6, MIR143HG, HNF1A-AS1, ZNF865, SPATS2, SLC25A32, CCL22, LOC100505876, ADAMTS4, RPS6KA3, TRIM16L, PDLIM5, MPL, ENTPD4, ADAT1, SAR1B, UCKL1-AS1, CYP1A2, METTL2B, ASB6, CEACAM22P, ABL2, LRRC27, ENAH, TP63, ZFAND5, LOC100506688, AK3, PPIEL, OCLN, WAC, SPATA5, TNFSF10, SCOC, LRRN4CL, BHMT2, PTAFR, GSTM3, FKBP5, TNFAIP8L2-SCNM1, PDK3, ZNF714, CXorf56, TRMT2B, CBFA2T2, SLC35E2, MTDH, EEF2K, HAUS2, TPMT, CEP68, SLC4A8, TSIX, CXADR, KLRD1, TMEM165, IDS, SS18, EFNB1, APOBEC3F, ADRA1A, SLC25A15, CLCC1, CBX5, STYX, RBBP5, GNG4, RBM34, ZNF829, RBM3, JAK3, FZD3, ZYG11A, ARHGAP1, PLEKHG2, NUP43, CXorf38, TRIM58, ZNF818P, SKA1, MTPAP, Clorf174, MFSD11, MAPKIIP1L, GA™-AS1, DESI1, IVNS1ABP, GLT25D1, C4orf19, GLG1, RFT1, ZNF626, LPIN3, CSF3, GPR155, SSR1, FCF1, ZNF737, NDUFV3, ATP1A1, EIF2S3, ATP6V1G1, TADA3, CLSPN, TBC1D15, RAB27A, HK1, RTCA, ARNTL2, KCNA7, SMIM12 and ZSWIM1; or IFI27, HLA-A, HLA-B, HLA-C, HLA-H, IFITM3, RPS26, ICAM1, IFITM1, IFI6, UBC, SERPINE1, TFPI2, EFNA1, TUBB2A, HLA-E, HLA-G, AHNAK2, TUBA1A, TUBB2B, POTEE, B2M, FN1, MBOAT1, TAPBP, KRT6C, RPS4Y1, TUBB4A, ORAI2, TOR1AIP2, SQSTM1, MT1L, RPL36A-HNRNPH2, TSPYL1, IFITM2, PDDC1, UBB, CHP1, CCL5, LYPLA1, NBPF10, ZBTB8A, PPP1CB, KCNQ1OT1, CYP20A1, ZNF490, IL1B, ANXA8L1, XPNPEP3, CTNNB1, LRTOMT, SHC1, STON2, LYZ, UGDH-AS1, POTEM, MDM2, NOL9, HLA-F, IKZF3, TNFAIP8L1, PPM1K, SLC3A2, AP1S3, PRICKLE2-AS3, DFFA, POLH, IRF1, PAFAH1B2, PLEKHB2, XIAP, UGGT1, CTGF, LOC100131257, TRAPPC2, PDE6A, FBLIM1, PDE4C, PRR11, PNPO, C17orf75, SRP9, RBMS2, CCDC142, AXL, ZNF793, KRT6A, TMEM41B, RAP1B, GNE, RBM3, FOXK1, INHBA, NMNAT1, IRGQ, TLCD2, BST2, NF2, TRAF3IP2, CA5B, SENP5, NUDT19, LINC00294, HOOK3, CYP4V2, LOC646214, CACUL1, PHACTR4, VHL, ZNF526, ZNF264, RUNDC1, HYDIN2, FBXL18, SPN, GDPD1, PHAX, TAF8, LRRC57, METTL21A, IVD, MGST3, SMYD4, ANXA8L2, PLTP, TVP23C, TM4SF1, ZBTB3, FKBP9L, MDM4, C6orf62, ILF3-AS1, TRPV1, LETM1, CHST6, TNFRSF10B, S1PR2, KIF18B, CPT1A, MOG, RNF41, SOX4, PLCXD1, PGAM5, LPP, GNG12, MEFV, ZNF850, OPA3, SCAI, TMEM33, LYRM7, SNAI2, SCOC, DSG3, LOC148709, SGTB, GRN, ATCAY, SYPL1, KREMEN1, BNIP3, ANGPTL4, RAMP2-AS1, MAVS, ZC3H12D, P2RX5-TAX1BP3, CDKN1A, ISG20, ANKRD20A9P, ZNF805, LOC284454, HLA-L, AGMAT, AKIRIN1, MYL12B, TANGO2, ACTG1, LOC284023, COX18, ORC4, PHLDA1, DNAL1, FAM3C, PGK1, ZNF430 and CCND2.

In certain embodiments, the modulating agent according to any embodiment herein is an antibody, or antigen binding fragment, an aptamer, affimer, non-immunoglobulin scaffold, small molecule, or genetic modifying agent.

In another aspect, the present invention provides for an isolated barrier tissue cell characterized by expression of one or more markers from one of clusters 0 to 21 in Table 1.

In certain embodiments, the isolated barrier tissue cell is a basal cell, the basal cell characterized by expression of one or more markers selected from: clusters 2, 8, and 12 of Table 1; selected from the group consisting of; S100A2, KRT5, KRT15, POSTN, MMP10, PERP, AQP3, EGR1, CD9, MIR205HG, F3, FOS, TACSTD2, KRT17, ALOX15, ETS2, JUNB, KRT19, DST, TNC, TSC22D1, ID1, TP63, LAMB3, CLDN1, IL33, ALDH3A1, SERPINFI, NCOA7, BTF3, FXYD3, PRSS23, ALDH3A2, SFN, CYR61, ATF3, SGK1, RPL10A; selected from the group consisting of; POSTN, S100A2, KRT5, KRT15, JUNB, MMP10, EGR1, MIR205HG, KRT17, TNC, RPL3, ETS2, DST, SERPINFI, TP63, RPL13A, RPS25, EIF1, IFITM3, IL33, LAMB3, RPL10A, RPL4, BTF3, RPS9; or selected from the group consisting of; KRT5, TSC22D1, KRT15, S100A2, DST, ALDH3A2, MIR205HG, CLDN1, TP63, KRT17, RASSF6, CYR61, ETS2, ADH7, MPZL2, BCAM, SLC6A6, PDPN, TNC, SFN, LAMB3, NTRK1, NTRK2, and SPINK5.

In certain embodiments, the isolated barrier tissue cell is a fibroblast cell, the fibroblast cell characterized by expression of one or more markers selected from: clusters 5 and 14 of Table 1; or selected from the group consisting of: DCN, COL1A2, LUM, COL3A1, MGP, LGALS1, CALD1, IGFBP7, FBLN1, CPE, SPAR, VIM, POSTN, IFITM3, SFRP1, SFRP2, C1S, COL1A1, SERPINGI, AEBP1, PCOLCE, TAGLN, C1R, SEPP1, PPAP2B, CRABP2, TPM2, IGFBP6, THY1, CDH11, CXCL14, FGF7, SELM, TMSB4X, RARRES2, VCAN, PRRX1, CLDN11, TPM1, NNMT, IGFBP4, BGN, LAPTM4A, PDGFRA, COL6A2, MXRA8, LIMA1, S100A6, APOD, FSTL1, LAMPS, NBL1, THBS1, EID1, IL6ST, KCNE4, IGF2, COL6A1, CCL2, MFAP4, COL15A1, ITGBL1, COL8A1, GLIPR1, TIMP3, RGS5, MYL9, ITGB1, APP, RARRES1, SERPINF 1, TGM2, and CLU.

In certain embodiments, the isolated barrier tissue cell is a myeloid cell, the myeloid cell characterized by expression of one or more markers selected from: cluster 11 of Table 1; or selected from the group consisting of: HLA-DRA, CD74, HLA-DRB1, SRGN, HLA-DPB 1, HLA-DPA1, TMSB4X, FTH1, AIF1, TMSB10, TYROBP, FTL, CST3, GPR183, HLA-DQA1, IFI30, HLA-DRB5, FGL2, ACTB, CTSS, IL8, PLAUR, LAPTM5, HLA-DQB1, PSAP, MS4A6A, FCER1G, NFKBIA, COTL1, DUSP2, HLA-DMA, IL1B, CPVL, MNDA, NAMPT, VIM, RGS2, CD83, PTPRC, ITGB2, SH3BGRL3, PLEK, LST1, TNFAIP3, OAZ1, BCL2A1, HLA-DMB, CLEC10A, LCP1, GPX1, F13A1, NPC2, TPM3, AMICA1, PFN1, HLA-B, CCL3, SAMSN1, ZNF331, ARPC1B, CYBB, NR4A2, PPP1R15A, ARPC2, RGS1, ARPC5, ARHGDIB, HLA-E, CTSH, CD68, CTSB, CD14, ACTR2, IGSF6, ARPC3, PTPRE, CFL1, ATP5E, CD52, CLEC7A, GRB2, MS4A7, SAMHD1, C5AR1, S100A4, CXCL2, CTSC, AREG, SOD2, S100A9, FCGRT, PABPC1.

In certain embodiments, the isolated barrier tissue cell is an apical cell, the apical cell characterized by expression of one or more markers selected from: clusters 0, 1, and 4 of Table 1; or selected from the group consisting of: SERPINB3, KRT19, S100A6, AGR2, ANXA, CLDN4, ELF3, SLPI, WFDC2, PRSS23, KRT8, TACSTD2, HSPB1, VMO1, SAT1, KRT18, TSPAN1, GSTP1, SERPINB4, AQP3, UGT2A2, EPAS1, ALDH1A1, LGALS3, ANXA2, PERP, EZR, CD9, TXN, ATP1B1, F3, MGST1, ALCAM, CXCL17, MT1X, FXYD3, PRDX1, S1OOP, GABRP, NTS, CSTB, ALOX15, CTG1, KRT7, CP, HES1, S100A11, DUSP1, CTSB, CLDN7, ATF3, ADAM28, KLF5, TNFSF10, SPINT2, CST1, and CD55.

In certain embodiments, the isolated barrier tissue cell is a glandular epithelium cell, the glandular epithelium cell characterized by expression of one or more markers selected from: clusters 3 and 13 of Table 1; selected from the group consisting of: LYZ, SLPI, AZGP1, PIGR, BPIFB1, LTF, ZG16B, STATH, TCN1, BPIFA1, PIP, C6orf58, DMBT1, RP11-1143G9.4, ODAM, XBP1, CXCL17, RNASE1, WFDC2, CCL28, NUCB2, NDRG2, SLC12A2, SCGB3A1, CA2, EHF, FAM3D, LRRC26, AQP5, PHLDA1, TMED3, PART1, CST3, PPP1R1B, MSMB, CLDN10, KIAA1324, FDCSP, P4HB, PRR4, HP, and MT-ND3; or selected from the group consisting of: LYZ, AZGP1, LTF, ZG16B, STATH, TCN1, BPIFB1, PIGR, SLPI, BPIFA1, PIP, C6orf58, DMBT1, RP11-1143G9.4, ODAM, XBP1, RNASE1, NUCB2, CCL28, SEC11C, SSR4, SCGB3A1, NDRG2, CA2, PHLDA1, CST3, CXCL17, LRRC26, SLC12A2, PPP1R1B, PART1, TMED3, FDCSP, FAM3D, PRR4, and HP; or selected from the group consisting of: LTF, ZG16B, STATH, AZGP1, TCN1, C6orf58, DMBT1, PIP, RP11-1143G9.4, ODAM, FDCSP, LPO, MUC5B, SCGB3A1, CCL28, CA2, NDRG2, SLC12A2, LRRC26, HP, PART1, PPP1R1B, CLDN10, and S100A1.

In certain embodiments, the isolated barrier tissue cell is a differentiating/secretory cell, the differentiating/secretory cell characterized by expression of one or more markers selected from: Table 15; or the group consisting of: VMO1, LYPD2, PSCA, SCGB1A1, S1OOP, MSMB, ALCAM, KRT7, KRT8, MGST1, MUC5AC, SPDEF, LCN2, CLDN7, PI3, SLC31A1, GABRP, TMEM213, STEAP4, GDF15, S100A14, MSLN, SORD, NTS, MUC16, ST6GALNAC1, SLC9A3R1, MUC1, CST1, CST4, IGFBP3, SLC6A14, CST2, CDH26, EGLN3, and POR.

In certain embodiments, the isolated barrier tissue cell is a ciliated cell, the ciliated cell characterized by expression of one or more markers selected from: cluster 16 of Table 1; or selected from the group consisting of: CAPS, C9orf24, TSPAN1, PIFO, TPPP3, C20orf85, SNTN, FAM183A, TUBB4B, TUBA1A, GSTA1, Cllorf88, RSPH1, PRDX5, OMG, AGR3, CAPSL, CIB1, CCDC170, DYNLT1, HSP90AA1, IFT57, DNAH5, DYNLL1, EZR, TMEM190, Clorf194, NUCB2, CALM1, ATPIF1, MORN2, RP11-356K23.1, PSENEN, SPA17, C9orf116, ZMYND10, ROPN1L, CETN2, LRRIQ1, DNAH12, C5orf49, PLAC8. TMC5, GSTP1, CCDC146, Clorf173, CALM2, CYP4B1, CHST9, TCTEX1D4, ARL3, CD59, FAM216B, SPAG6, FAM154B, FAM81B, FAM229B, SMIM22, EFCAB1, NQO1, ABCA13, IK, ARMC3, FOXJ1, CDHR3, SCGB2A1, IQCG, PRDX1, RRAD, ANXA1, TSPAN19, PCM1, FAM92B, DYDC2, RSPH4A, SLC44A4, UFC1, DNALI1, CKB, NME5, TEKT1, ODF3B, C9orf135, ALDH1A1, LGALS3, WDR78, ODF2L, HSPH1, ALDH3B1, TSPAN6, LRRC23, WDR52, CTSS, MS4A8, SPAG16, ENKUR, EFHC1, PSCA, NUDC, HMGN3, ZBBX, MLF1, KIF21A, RSPH9, Clorf192, CCDC11, CCDC113, AK7, AKAP9, LDLRAD1, WDR54, KIF9, EFCAB10, WDR96, C12orf75, DYNLRB2, HSPB11, FXYD3, TSTD1, HSBP1, AKAP14, WDR86-AS1, C10orf107, Cllorf70, CES1, MNS1, SPEF2, SPATA18, CCDC17, NPHP1, DPY30, TAX1BP1, TCTEX1D2, ARHGAP18, PPIL6, C14orf142, C21orf59, GSTA2, CCDC19, TMEM231, C6orf118, STOML3, FANK1, SEPW1, SPAG1, ALCAM, ANXA2, CSPP1, DHRS9, MRPS31, TSPAN3, CYSTM1, RP11-867G2.2, SRI, NEK10, ANKUB1, SYNE1, DPCD, CATSPERD, CCDC39, NWD1, MORN5, CD164, CLDN7, S100A6, SAMHD1, DNPH1, SPAG17, RP11-275114.4, B9D1, WDR66, LRRC46, MAP3K19, LRRC48, EFCAB2, AGR2, LINC0094, DNAAF1, PROM1, DNAJA4, CDS1, C9orf117, FHAD1, DNAH3, OSCP1, FAM174A, H2AFJ, WFDC2, PIH1D2, RABL5, PERP, IFI27, CCDC173, IGFBP2, SAT1, DTHD1, CCDC42B, DNAH9, CCDC176, LZTFL1, SOD1, CLU, CCDC65, C11orf74, CTGF, DRC1, CASC1, DSTN, TRAF3IP1, CCDC104, YWHAE, COX6A1, TMBIM6, IFT172, SLC27A2, LRP11, S100A11, ALOX15, IFT43, TXN, STK33, ARMC4, DZIP3, RAB11FIP1, UBXN10, IFT81, IGFBP7, TTC18, CYB5A, CAST, TMEM59, ELF3, UBB, DNAH11, C7orf57, PTGES3, TTC29, PPP1R42, CLDN3, MUC16, TUSC3, TCTN1, POLR2I, CCDC78, RUVBL2, TNFAIP8L1, CC2D2A, GDF15, TAGLN2, CDHR4, DNAL1, ECT2L, RUVBL1, SYAP1, METTL7A, DNAH7, IQCD, NDUFB1, UBL5, RP4-666F24.3, C9orf9, C21orf58, ANKRD66, EPCAM, PCDP1, CMPK1, TMEM14B, MORF4L2, and MT-RNR1; or from the group consisting of: CAPS, C9orf24, PIFO, TPPP3, C20orf85, SNTN, FAM183A, Cllorf88, RSPH1, CAPSL, TMEM190, Clorf194, MORN2, RP11-356K23.1, SPA17, C9orf116, ZMYND10, ROPN1L, LRRIQ1, DNAH12, C5orf49, CCDC146, Clorf173, TCTEX1D4, FAM216B, SPAG6, FAM154B, FAM81B, FAM229B, SMIM22, EFCAB1, ARMC3, FOXJ1, CDHR3, IQCG, RRAD, TSPAN19, FAM92B, DYDC2, RSPH4A, DNALI1, NME5, TEKT1, ODF3B, and C9orf135.

In certain embodiments, the isolated barrier tissue cell is a plasma cell, the plasma cell characterized by expression of one or more markers selected from: clusters 7, 10, 15, and 17 of Table 1; or selected from the group consisting of: IGJ, SSR4, MZB1, IGHA1, SEC11C, HSP90B1, ENAM, IGHG1, IGHA2, IGHG4, IGHG3, HERPUD1, DERL3, PRDX4, IGHG2, FKBP11, IGKC, AC096579.7, SPCS3, RGS1, TSC22D3, SLAMF7, FAM46C, SSR3, PIM2, RNA28S5, CD79A, XBP1, ITM2C, IGLC7, SEL1L, IGLL1, FCRL5, PRDM1, TRAM1, UBE2J1, RRBP1, SUB1, SPCS1, CCND2, IGLC3, ERLEC1, FKBP2, SPCS2, SELK, IGKV3-20, ISG20, CYBA, and C19orf10.

In certain embodiments, the isolated barrier tissue cell is an endothelial cell, the endothelial cell characterized by expression of one or more markers selected from: cluster 6 from Table 1; or from the group consisting of: SPARCL1, VIM, HLA-E, GNG11, A2M, TMSB10, IFI27, IGFBP7, IFITM3, CD74, CLDN5, ELTD1, TMSB4X, DARC, EMCN, TM4SF1, SPARC, PTRF, VWF, GIMAP7, IFITM2, PLVAP, RPS23, RPL3, ECSCR, RPL32, EMP1, HLA-DRB1, RAMP2, CALCRL, PTMA, HLA-DRA, RPL31, ESAM, ID3, APOLD1, FKBP1A, ADAMTS1, ADIRF, RPS27A, RAMP3, RPS15A, EGFL7, HLA-B, RPS13, IL33, RPS3, LIFR, CAV1, NPDC1, CD34, AC011526.1, RPS9, NOSTRIN, RPL24, IL6ST, CYYR1, CRIP2, RDX, RPL5, JAM2, TGFBR2, STOM, TPM3, TXNIP, HLA-DPA1, RPL26, TSPAN7, ENG, SPRY1, EEF1A1, PALMD, SPTBN1, RPL9, CD93, ELK3, SOCS3, MYL12A, RPL19, SELE, KCTD12, RPL12, HLA-DRB5, and RPL10.

In certain embodiments, the isolated barrier tissue cell is a T cell, the T cell characterized by expression of one or more markers selected from: cluster 9 of Table 1; or the group consisting of: TMSB4X, RPS29, RPS27A, CD52, RPS15A, RPS27, CXCR4, RPS25, SRGN, PTPRC, TRBC2, RPL13A, RPS20, HLA-B, IL32, CCL5, RPL32, RPL23A, CD2, RPS3, RPL37, CD3D, IL7R, HLA-E, PFN1, ARHGDIB, RPL39, TSC22D3, PTPRCAP, CD69, RPL14, RGS1, RPL10, HLA-C, HLA-A, S100A4, TNFAIP3, LCP1, UBA52, ETS1, KLRB1, and GZMA.

In certain embodiments, the isolated barrier tissue cell is a mast cell, the mast cell characterized by expression of one or more markers selected from: TPSAB1, CPA3, CD69, SRGN, HPGD, RGS1, HPGDS, SLC18A2, SAMSN1, KIT, NFKBIZ, HDC, CTSG, ACSL4, FTH1, LAPTM5, TMSB4X, TNFAIP3, TPSD1, CD52, PTGS2, GATA2, NFKBIA, PPP1R15A, IL1RL1, VIM, FTL, DUSP6, AHR, MS4A2, CD63, NR4A2, Clorf186, VWA5A, CLU, AREG, SELK, RGS2, CCL4, ANXA1, ALOX5AP, GPR65, TYROBP, GLUL, RGS13, S100A4, FOSB, CAPG, UBB, TSC22D3, FCER1G, PTMA, GCSAML, ALAS1, CTSD, NR4A1, KLF6, RAC2, BTG, ARHGDIB and RP11-354E11.2.

In another aspect, the present invention provides for a method of modulating barrier cell proliferation, differentiation, maintenance and/or function, comprising: contacting a barrier cell or population of barrier cells with a barrier cell modulating agent in an amount sufficient to modify differentiation, maintenance, and/or function of the barrier cell or population of barrier cells as compared to differentiation, maintenance, and/or function of the barrier cell population or population of barrier cells in the absence of the barrier cell modulating agent.

In certain embodiments, contacting the barrier cell or population of barrier cells with the barrier cell modulating agent rebalances barrier cell type diversity relevant to normal and/or non-diseased barrier cell tissue

In certain embodiments, modulating barrier cell proliferation, differentiation, maintenance and/or function is used to treat or prevent type 2 inflammatory responses in barrier tissues.

In certain embodiments, the barrier cell modulating agent modulates one or more of any of the targets: KRT5, KRT8, FOXJ1, LTF, DARC, COL1A2, CD79A, HLA-DRA, TRBC2, TPSAB1, CCL26, CCL11, CCL24, CXCL17, CCL28, HPGDS, PTGS2. ALOX5, ALOX15, IL-18, IL-1B, TP63, SCGB1A1, PIFO, MUC5B, MSMB, SCGB3A1, PSCA, LYPD2, CST4, CST1, TFF3, POSTN, PTHLH, SERPINB2, HS3ST1, CDH26, MMP10, SPINK5, ALDH3A1, CLCA4, GLUL, WNT, Notch, ITGA8, FN1, EPAS1, NTRK2, FGFBP1, ETS2, CSRP2, SULTIE1, FAM107A, PTN, ID2, EGFR, PAPPA, NNMT, COL1A1, LAMB1, ABCA5, LIMA1, TRAPPC3L, SMOC1, COL16A1, DCBLD2, RGS4, SLC39A6, EFEMP1, OXTR, PLOD2, LINC00152, TNFRSF12A, SERPINE1, CALU, TPM4, MPP4, RHOJ, FAM65C, ABCA8, KANK4, FGF10, FGFBP2, ABCA3, HUNK, PRB4, TENM1, CLMN, RIC3, BPIFB2, CCBE1, ATP1A2, CNTN3, COL11A1, PNISR, CCL2, CXCL12, CCL5, CCL13, IL-6, IL-10, IGFBP3, STEAP4, or EGLN3. In certain embodiments, the one or more modulating agents modulate at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104 or more of the targets, or at least (or equal to) 1-5, 5-10, 10-20, 20-50, 50-110 of the targets.

In certain embodiments, the barrier cell modulating agent modulates one or more of any of the targets: TBXAS1, MUC5AC, FOXA3, SPDEF, IGFB3, ELGN3, LGR6, CD44, p63, FOXA1, Bach2, Sox, IF144L, AP-1, STAT, CCL17, CCL18, PTGES, PTGDS, TGFB2, TNF, S100A8, or S100A9. In certain embodiments, the one or more modulating agents modulate 2, 3, 4, 5, 6, 7, 8, 9, 10, 11, 12, 13, 14, 15, 16, 17, 18, 19, 20, 21, 22, or 23 of the targets.

In certain embodiments, the barrier cell modulating agent modulates one or more of the genes in Table 1, Table 2, Table 3, or a combination thereof. In certain embodiments, the barrier cell modulating agent modulates one or more of any of CCL26, CCL11, CCL24, CXCL17, CCL28. In certain embodiments, barrier cell modulating agent inhibits expression of: CCL26 in the basal cells of claim 24 and/or the fibroblasts of claim 25; CCL11 in the fibroblasts of claim 25; CCL24 in the myeloid cells of claim 26; CXCL17 in the apical cells of claim 27 and/or the glandular epithelium cells of claim 6; CCL28 in the glandular epithelium of claim 28; or a combination thereof.

In certain embodiments, the barrier cell modulating agent modulates one or more of HPGDS, PTGS2, ALOX5, and TBXAS. In certain embodiments, the barrier cell modulating agent inhibits expression of: HPGDS, PTGS2, and/or ALOX5 expression in the mast cells of claim 34; or TBXAS expression in the myeloid cells of claim 26.

In certain embodiments, the barrier cell modulating agent inhibits IL-33 and/or TSLP expression or prevents IL-33 or TSLP from binding to a corresponding receptor. In certain embodiments, the barrier cell modulating agent inhibits: IL-33 expression in the basal cells of claim 24, the apical cells of claim 27, and/or the ciliated cells of claim 30; or TSLP expression in the basal cells of claim 24.

In certain embodiments, the barrier cell modulating agent inhibits IL4, IL5, IL13, HPGDS, and/or AREG expression, or prevents IL4, 115, IL13, HPGDS, and/or AREGfrom binding to a corresponding receptor. In certain embodiments, the barrier cell modulating agent inhibits: IL4, IL15, IL13, and/or HPGDS expression in the T cells of claim 33; IL5 and IL13 expression in the mast cells of claim 34; AREG expression in the mast cells of claim 34 and/or the myeloid cells of claim 4; or a combination thereof.

In certain embodiments, the barrier cell modulating agent either; inhibits expression of one or more of CST4, CST1, IGFBP3, TFF3, and ELGN3 in the glandular epithelium cells of claim 6 the differentiating/secretory cells of claim 7, or the apical cells of claim 27; or induces increased expression of MSMB, SCGB1A1, STEAP4, PSCA, LYPDL in the glandular epithelium cells of claim 6 the differentiating/secretory cells of claim 7, or the apical cells of claim 27; or a combination thereof.

In certain embodiments, the barrier cell modulating agent increases production of secreted mucins in the barrier tissue. The secreted mucins may be MUC5B and MUC5AC. In certain embodiments, the barrier cell modulating agent increases: production of MUC5B and MUC5AC+ by increasing expression of SPDEF; or production of MUC5AC+ by increasing expression of SCGB1A1 and/or FOXA3.

In certain embodiments, the barrier cell modulating agent induces differentiation of basal cells.

In certain embodiments, the barrier cell modulating agent inhibits expression of one or more of DLK2, DLL1, JAG2, DKK3, POSTN, FN1, and TNC in the basal cells of claim 24.

In certain embodiments, the barrier cell modulating agent either: inhibits expression of one or more AP-1 transcription factor family members, including JUN, FOXA1, BACH2, and p63; or increases expression of one or more SOX/STAT/MEF2 transcription factor family members; or a combination thereof.

In certain embodiments, the barrier cell modulating agent induces differentiation of basal cells.

In another aspect, the present invention provides for a method of modulating basal cell proliferation, differentiation, maintenance and/or function comprising administering, to the basal cell, an effective amount of one or more modulating agents able to interact with one or more genes or gene expression products in Table 4. In certain embodiments, the one or more genes or gene expression products is one or more of POSTN, PTHLH, ALOX15, SERPINB2, HS3ST1, CDH26, MMP10, CCL26, SPINK5, ALDH3A1, CLCA4, and/or GLUL. In certain embodiments, the one or more of POSTN, PTHLH, ALOX15, SERPINB2, HS3ST1, CDH26, MMP10, or CCL26, is decreased or one or more of SPINK5, ALDH3A1, CLCA4, or GLUL is increased, relative to prior to administration of the effective amount of one or more modulating agents.

In another aspect, the present invention provides for a method of modulating basal cell proliferation, differentiation, maintenance and/or function comprising administering, to the basal cell, an effective amount of one or more modulating agents able to interact with one or more of KRT5, IL-33, TSLP, and/or TP63. In certain embodiments, the one or more of KRT5, IL-33, TSLP, and/or TP63 is increased relative to prior to administration of the effective amounts of one or more modulating agents.

In another aspect, the present invention provides for a method of modulating endothelial cell proliferation, differentiation, maintenance and/or function comprising administering, to the endothelial cell, an effective amount of one or more modulating agents able to interact with one or more genes or gene expression products in Table 5. In certain embodiments, the one or more genes or gene expression products is MSMB. In certain embodiments, MSMB is increased relative to prior to administration of the effective amounts of one or more modulating agents.

In another aspect, the present invention provides for a method of modulating endothelial cell proliferation, differentiation, maintenance and/or function comprising administering, to the endothelial cell, an effective amount of one or more modulating agents able to interact with one or more genes or gene expression products, wherein the one or more genes or gene expression products is one or more of IL-33, IL-18, SCGB1A1, SCGB3A1, PSCA, or LYPD2. In certain embodiments, the one or more of IL-33 or IL-18 is decreased and/or one or more of, SCGB1A1, SCGB3A1, PSCA, and/or LYPD2 is increased relative to prior to administration of the effective amounts of one or more modulating agents.

In another aspect, the present invention provides for a method of modulating endothelial cell proliferation, differentiation, maintenance and/or function comprising administering, to the endothelial cell, an effective amount of one or more modulating agents able to interact with one or more genes or gene expression products, wherein the one or more genes or gene expression products is DARC. In certain embodiments, DARC is increased relative to prior administration of the effective amounts of one or more modulating agents.

In another aspect, the present invention provides for a method of modulating fibroblast cell proliferation, differentiation, maintenance and/or function in barrier tissues comprising administering, to the fibroblast cell, an effective amount of one or more modulating agents able to interact with one or more genes or gene expression products in Table 6. In certain embodiments, the one or more genes or gene expression products is one or more of CCL26 and/or CCL11. In certain embodiments, the one or more of CCL26 or CCL11 is decreased relative to prior to administration of the effective amounts of one or more modulating agents.

In another aspect, the present invention provides for a method of modulating fibroblast cell proliferation, differentiation, maintenance and/or function in barrier tissues comprising administering, to the fibroblast cell, an effective amount of one or more modulating agents able to interact with one or more genes or gene expression products wherein the one or more genes or gene expression products is one or more of COL1A2, and/or ITGA8. In certain embodiments, the one or more of COL1A2, and/or ITGA8 is increased/decreased relative to prior to administration of the effective amounts of one or more modulating agents.

In another aspect, the present invention provides for a method of modulating macrophage cell proliferation, differentiation, maintenance and/or function in barrier tissues comprising administering, to the macrophage cell, an effective amount of one or more modulating agents able to interact with one or more genes or gene expression targets in Table 7.

In another aspect, the present invention provides for a method of modulating mast cell proliferation differentiation, maintenance and/or function in barrier tissues comprising administering, to the macrophage cell, an effective amount of one or more modulating agents able to interact with one or more genes or gene expression targets in Table 8. In certain embodiments, the one or more genes or gene expression products is one or more of HPGDS, PTGS2, or AREG. In certain embodiments, the one or more of HPGDS, PTGS2, or AREG is decreased relative to prior to administration of the effective amounts of one or more modulating agents.

In another aspect, the present invention provides for a method of modulating mast cell proliferation differentiation, maintenance and/or function in barrier tissues comprising administering, to the mast cell, an effective amount of one or more modulating agents able to interact with one or more genes or gene expression targets, wherein the one or more genes or gene expression products is TPSAB1, IL-5, ALOX5 and/or IL-13. In certain embodiments, the one or more of TPSAB1, IL-5, ALOX5, and/or IL-13 is increased/decreased relative to prior to administration of the effective amounts of one or more modulating agents.

In another aspect, the present invention provides for a method of modulating plasma cell proliferation, differentiation, maintenance and/or function in barrier tissues comprising modulating one or more genes or gene expression targets in Table 9.

In another aspect, the present invention provides for a method of modulating T cell proliferation, differentiation, maintenance and/or function in barrier tissues comprising modulating one or more genes or gene expression targets in Table 10.

In certain embodiments, the barrier modulating agent according to any embodiment herein comprises a CRISPR system, a zinc finger system, TALE, TALEN, therapeutic antibody, bi-specific antibody, antibody fragment, antibody-like protein scaffold, aptamer, RNAi or small molecule.

In another aspect, the present invention provides for a method of identifying a basal cell comprising detecting one or more genes or gene expression products of claim 2. In certain embodiments, the gene or gene expression products are one or more of POSTN, PTHLH, ALOX15, SERPINB2, HS3ST1, CDH26, MMP10, CCL26, SPINK5, ALDH3A1, CLCA4, or GLUL. In certain embodiments, a basal cell in diseased tissue is identified by detecting an increase in one or more of POSTN, PTHLH, ALOX15, SERPINB2, HS3ST1, CDH26, MMP10, or CCL26, and/or a decrease in one or more of SPINK5, ALDH3A1, CLCA4, or GLUL compared to a basal cell from non-disease tissue.

In another aspect, the present invention provides for a method of identifying a basal cell comprising detecting one or more genes or gene expression products, wherein the one or more genes or gene expression products is one or more of KRT5, IL-33, TSLP, and/or TP63. In certain embodiments, the one or more of KRT5, IL-33, TSLP, and/or TP63 is increased compared to a basal cell from non-disease tissue.

In another aspect, the present invention provides for a method of identifying an endothelial cell comprising detecting one or more genes or gene expression products in Table 5. In certain embodiments, the one or more genes or gene expression products is MSMB. In certain embodiments, MSMB is decreased compared to an endothelial cell from non-disease tissue.

In another aspect, the present invention provides for a method of identifying an endothelial cell comprising detecting one or more genes or gene expression products, wherein the one or more genes or gene expression products is one or more of IL-33, IL-18, SCGB1A1, SCGB3A1, PSCA, or LYPD2. In certain embodiments, an endothelial cell from diseased tissue is identified by detecting an increase in one or more of IL-33 or IL-18, and/or a decrease in one or more of SCGB1A1, SCGB3A1, PSCA, or LYPD2 compared to an endothelial cell from non-disease tissue.

In another aspect, the present invention provides for a method of identifying an endothelial cell comprising detecting one or more genes or gene expression products, wherein the one or more genes or gene expression products is DARC. In certain embodiments, DARC is increased/decreased compared to an endothelial cell from non-disease tissue.

In another aspect, the present invention provides for a method of identifying a fibroblast cell comprising detecting one or more genes or gene expression products in Table 6. In certain embodiments, the one or more genes or gene expression products is one or more of CCL26 and/or CCL11. In certain embodiments, a fibroblast from disease tissue is identified by detecting an increase in one or more of CCL26 and/or CCL11 compared to a fibroblast from non-disease tissue.

In another aspect, the present invention provides for a method of identifying a fibroblast cell comprising detecting one or more genes or gene expression products, wherein the one or more genes or gene expression products is one or more of COLIA2, and/or ITGA8.

In another aspect, the present invention provides for a method of identifying a macrophage cell comprising detecting one or more target genes or target gene expression products in Table 7.

In another aspect, the present invention provides for a method of identifying a mast cell comprising detecting one or more target genes or target gene expression products in Table 8. In certain embodiments, the target gene or target gene expression products are one or more of HPGDS, PTGS2, AREG. In certain embodiments, a mast cell from disease tissue is identified by detecting an increase in one or more of HPGDS, PTGS2, or AREG compared to a mast cell from non-disease tissue.

In another aspect, the present invention provides for a method of identifying a mast cell comprising detecting one or more genes or gene expression targets, wherein the one or more genes or gene expression targets is TPSAB1, IL-5, ALOX5 and/or IL-13. In certain embodiments, the one or more of TPSAB1, IL-5, ALOX5 and/or IL-13 is increased/decreased compared to a mast cell from non-disease tissue.

In another aspect, the present invention provides for a method of identifying a plasma cell comprising detecting one or more genes or gene expression targets in Table 9.

In another aspect, the present invention provides for a method of identifying a T cell comprising detecting one or more genes or gene expression targets in Table 10.

In another aspect, the present invention provides for a method of treating, preventing or ameliorating chronic type 2 inflammation in barrier tissue comprising administering a modulating agent to a subject in need thereof, wherein the modulating agent restores cell diversity in epithelial tissue. In certain embodiments, restoring cell diversity in epithelial tissue comprises inducing basal cell differentiation. In certain embodiments, the modulating agent inhibits IL4, IL5 IL13, and/or NGF. In certain embodiments, the modulating agent inhibits WNT. In certain embodiments, the modulating agent increases expression of Notch, DLL1, DLL2, or a combination thereof.

In another aspect, the present invention provides for a method of detecting a type 2 inflammation induced tissue disease comprising: producing a single cell transcriptome/proteome for one or more cells from a first population of cells in a first sample; analyzing the transcriptome/proteome from the first sample to determine sub-populations of cells in the first sample; comparing the transcriptome/proteome of the one or more cells from the first population of cells with a transcriptome/proteome from a second population of cells; and determining a difference in the expression of any one of the following genes: i) one or more of IL4, IL5, or IL13; ii) Wnt or Notch; iii) POSTN, PTHLH, ALOX15, SERPINB2, HS3ST1, CDH26, MMP10, CCL26, SPINK5, ALDH3A1, CLCA4, or GLUL; iv) HPGDS, PTGS2, ALOX5, OR AREG; v) IL-33, IL-18, MSMB, SCGB1A1, SCGB3A1, PSCA, or LYPD2; or CCL26 and CCL11; [0077] wherein an increase in the expression of any one of: i) one or more of IL4, IL5, or IL13; ii) Wnt; iii) POSTN, PTHLH, ALOX15, SERPINB2, HS3ST1, CDH26, MMP10, CCL26; iv) HPGDS, PTGS2, ALOX5, OR AREG; or wherein a decrease in the expression of any one of: i) Notch; ii) SPINK5, ALDH3A1, CLCA4, or GLUL; iii) MSMB, SCGB1A1, SCGB3A1, PSCA, or LYPD2, or iv) CCL26 and CCL11; or v) one or more markers from non-polyp cluster of Table 15 is indicative of the type 2 inflammation induced tissue disease.

In certain embodiments, the method further comprises producing a single cell transcriptome/proteome for one or more cells from a second population of cells in a second sample to thereby provide transcriptome/proteome from the second population of cells. In certain embodiments, the transcriptome/proteome from the second population of cells is a reference sample, standard or a control. In certain embodiments, the tissue disease is nasal polyps. In certain embodiments, the first and second samples are from the same individual organism. In certain embodiments, the second sample is a reference sample, optionally from a second individual. In certain embodiments, t-distributed stochastic neighbor embedding is used to analyze the transcriptomes/proteome to determine sub-populations of cells.

In another aspect, the present invention provides for a method of modulating basal cell proliferation, differentiation, maintenance and/or function comprising administering, to the basal cell, an effective amount of one or more modulating agents able to interact with an AP-1 transcription family member. In certain embodiments, the one or more modulating agent inhibits the AP-1 transcription family member, wherein inhibition of the AP-1 transcription family member induces differentiation of the basal cell. In certain embodiments, the AP-1 transcription family member is p63, FOXA1, or Bach2.

In another aspect, the present invention provides for a method of determining cellular diversity in barrier tissue samples comprising: detecting an amount of basal, fibroblast, myeloid, apical, glandular epithelium, differentiating/secretory, ciliated, plasma, endothelial, mast, and T cells in a sample, by detecting one more markers corresponding to each cell type as defined in claims 24 to 34. In certain embodiments, the method further comprises detecting type 2 inflammation in in the sample by detecting a decrease in cellular diversity relative to a healthy barrier tissue reference. In certain embodiments, the decrease in cellular diversity comprises an increase in basal cells and/or a decrease in ciliated and glandular cells relative to the healthy barrier tissue reference.

In another aspect, the present invention provides for a method of detecting type 2 inflammation in barrier tissues comprising; detecting: i) an increase in expression of one or more of DLK2, DLL1, JAG2, DKK3, POSTN, FN1, and TNC relative to a healthy barrier tissue reference; ii) a decrease in SPINK5, ALDH3A1, CLCA4, an GLUL expression relative to a healthy barrier tissue reference; iii) an increase in expression of one or more of JUN, FOXA1, BACH2, and p63 relative to a healthy barrier tissue reference; or iv) a combination thereof.

In another aspect, the present invention provides for a method of characterizing a cell phenotype, cell signature, cell expression profile, or cell expression program, the method comprising detecting one or more expression products from Table 1, Table 2, Table 3, Table 4, Table 5, Table 6, Table 7, Table 8, Table 9, Table 10, Table 11, Table 12, Table 13, Table 14, Table 15, or combination thereof.

In certain embodiments, the cell phenotype, cell signature, cell expression profile, or cell expression program is microenvironment specific, the cell phenotype, cell signature, cell expression profile, or cell expression program is found in a particular spatio-temporal context; the cell phenotype, cell signature, cell expression profile, or cell expression program is specific to a particular pathological context; a combination of cell subtypes having a particular cell phenotype, cell signature, cell expression profile, or cell expression program indicates a therapeutic or diagnostic outcome; the cell phenotype, cell signature, cell expression profile, or cell expression program are used to deconvolute the network of cells present in a particular pathological condition; the presence of specific cells and cell subtypes are indicative of a particular response to treatment, optionally including increased or decreased susceptibility to treatment, optional by a particular pharmaceutical agent or mode of therapy; the cell phenotype, cell signature, cell expression profile, or cell expression program indicates the presence of one particular cell type.

In another aspect, the present invention provides for a method for screening for drugs that induce or reduce a cell phenotype, cell signature, cell expression profile, or cell expression program, optionally in immune cells, the method comprising detecting changes in one or more expression products from Table 1, Table 2, Table 3, Table 4, Table 5, Table 6, Table 7, Table 8, Table 9, Table 10, Table 11, Table 12, Table 13, Table 14, Table 15, or combination thereof. In certain embodiments, the cell phenotype, cell signature, cell expression profile, or cell expression program is used for GE-HTS (Gene Expression-based High-Throughput Screening) and, optionally, a pharmacological screen is used to identify drugs that selectively activate barrier cells.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—The inflamed human sinus cellular ecosystem by scRNA-seq provides a map for type 2 immunity mediators. FIG. 1a is an Illustration of the clinical disease spectrum sampled (n=12 samples) and the experimental workflow leading to the generation of a tSNE plot displaying 18,036 single cells, colored by clusters identified through shared nearest neighbor (SNN) analysis (Supplementary Table 3; Methods) from respiratory tissue. FIG. 1b is a tSNE plot of 18,036 single cells (n=12 samples), colored by cell types identified through marker discovery (ROC test) and biological curation of identified clusters (Supplementary Table 3; Methods). FIG. 1c is a graph showing select marker gene overlays displaying binned count-based (unique molecular identifier (UMI) collapsed) expression level (log(scaled UMI+1)) on a tSNE plot from FIG. 1b for key cell types identified (see Supplementary Table 3 for full gene lists); area under the curve (AUC) 0.998 to 0.7 for all markers displayed. FIG. 1d is a dot plot of chemokines and lipid mediators with known roles in type 2 immunity mapped onto cell types across all samples, dot size represents fraction of cells within that type expressing, and color intensity binned count-based expression level (log(scaled UMI+1)) amongst expressing cells (see FIG. 11 by disease state). FIG. 1e is a dot plot of inducers and effectors of type 2 immunity mapped onto cell types across all samples, dot size represents fraction of cells within that type expressing, and color intensity binned count-based expression level (log(scaled UMI+1)) amongst expressing cells (see FIG. 11 by disease state).

FIG. 2—Single-cell transcriptomes of epithelial cells in type 2 inflammation and stratification by disease state. FIG. 2a is a tSNE plot of 10,274 epithelial cells (n=12 samples), colored by clusters identified through SNN, with adjacent color bars representing related cell clusters based on marker genes from ROC-tests (AUC>0.65), hierarchical clustering, and scoring over basal cells (Cluster Numbers as in FIG. 1a,b; see Methods and FIG. 12). NB: epithelial data is not re-clustered to help in maintaining visual orientation, but is re-clustered in FIG. 13). FIG. 2b is a row-normalized heatmap of the top marker genes identified by ROC-test (AUC>0.65) for each epithelial cell type with select genes displayed on y-axis and cluster annotations on x-axis (see Supplementary Table 3 for full gene lists). FIG. 2c is a row-normalized heatmap of the top marker genes identified by ROC-test (AUC>0.6) within each cell type for each cell cluster with genes displayed on y-axis and cluster annotations on x-axis (see Supplementary Table 3 for full gene lists). FIG. 2d is a tSNE plot of 10,274 epithelial cells colored by disease state (Cluster Numbers as in FIG. 1a,b; n=6 non-polyp, n=6 polyp samples). NB: epithelial data is not re-clustered here to help in maintaining visual orientation, but is re-clustered limited to cells from same disease in FIG. 13). FIG. 2e shows violin plots for the count-based expression level (log(scaled UMI+1)) for key differentially expressed genes using bimodal test within the differentiating/secretory cell subset across disease states; n=6 non-polyp, n=6 polyp samples, *bimodal test, all p<2.03×10−55 or less with Bonferroni correction for multiple hypothesis testing based on number of genes tested.

FIG. 3—Nasal scrapings as a window into healthy nasal epithelium and shifts in cell state of secretory cells across health and disease. FIG. 3a shows a tSNE plot (left) of 18,704 single cells from nasal scrapings (n=9 samples) colored by clusters identified through shared nearest neighbor (SNN) analysis (Supplementary Table 3; Methods), a tSNE plot (middle) colored by cell types identified through marker discovery (ROC test) and biological curation of identified clusters (Supplementary Table 3; Methods), and a tSNE plot (right) colored by disease and tissue of origin from healthy inferior turbinate (7,603 cells; n=3 samples), polyp-bearing patient inferior turbinate (2,298 cells; n=4 samples), and polyp scraping directly from polyp in ethmoid sinus (8,803 cells; n=2 samples); with adjacent select marker gene overlays displaying count-based UMI-collapsed expression level (log(scaled UMI+1)) for apical epithelial (KRT8) and hematopoietic (PTPRC) genes. FIG. 3b shows select marker gene overlays displaying count-based UMI-collapsed expression level (log(scaled UMI+1)) on a tSNE plot from FIG. 3a for key cell types identified (see Supplementary Table 3 for full gene lists); area under the curve (AUC) 0.946 to 0.705 for all markers displayed. FIG. 3c shows violin plots for the count-based expression level (log(scaled UMI+1)) for key differentially expressed genes using ROC test within myeloid cells across disease states and tissues identified (Methods); 137 cells, n=3 healthy inferior turbinate; 157 cells, n=4 polyp inferior turbinate; 210 cells, n=2 polyp ethmoid sinus samples; *AUC 0.67 for TXNRD1, 0.615 for RALA, 0.647 for TLR2, 0.619 for RIPK2, 0.747 for C1QA, 0.674 for FGL2. FIG. 3d shows a tSNE plot (top) of 18,325 single cells from nasal scrapings (n=9 samples) merged and re-clustered with surgical samples (n=12 samples) colored by disease and tissue of origin from healthy inferior turbinate (3,681 cells, n=3 samples), polyp-bearing patient inferior turbinate (1,370 cells, n=4 samples), non-polyp ethmoid sinus surgical samples (5,928 cells, n=6 samples), and polyp surgical and scraping samples directly from polyp in ethmoid sinus (7,346 cells, n=8 samples), and a tSNE plot (bottom) colored by cell types identified through marker discovery (ROC test) and scoring based on deconvolution gene lists (Supplementary Table 3 used in FIGS. 4g and h; Methods) representing 3,152 basal, 3,089 differentiating, 8,840 secretory, 1,105 ciliated, and 2,139 glandular cells, NB: zero glandular cells were captured from scraping samples. FIG. 3e is a row-normalized heatmap on 1,000 sub-sampled secretory cells from each disease/location category of the top marker genes identified by ROC-test with select genes displayed on y-axis including a core secretory signature (ROC-test AUC>0.662 secretory cells vs. rest of cells), and then within secretory cells a ROC-test used to identify marker genes within each disease/location category, and cluster annotations on x-axis (see Supplementary Table 3 for full gene lists, all AUC>0.65 for markers displayed). FIG. 3f is a violin plot of expression contribution to a cell's transcriptome over IFNa, IFNy, and IL-4/IL-13 commonly-induced gene signature in secretory cells grouped as in (e) from each disease/location category (see Methods, Supplementary Table 4 for gene lists used; IFNa polyp ethmoid sinus vs healthy: effect size −1.16; IFNy polyp ethmoid sinus vs healthy: effect size −1.05; IL4/IL13 polyp ethmoid sinus vs. healthy: effect size 1.32; all p<2.2×10−16).

FIG. 4—The tissue ecological diversity of airway epithelial cells is reduced and basal cells are significantly increased in individuals with nasal polyps. FIG. 4a is a bar graph showing the frequency of each cell subset amongst all epithelial cells recovered in scRNA-seq calculated for each sample; n=6 non-polyp, 6 polyp samples, *t-test p<0.05 for indicated cell types, non-polyp vs polyp. FIG. 4b shows Simpson's index of diversity over epithelial cell types, an indication of the total richness present within an ecosystem, calculated for each sample; points represent individual samples, *t-test p<0.0021. FIG. 4c demonstrates representative gating strategy of flow cytometry performed on single cell suspensions of cells used to identify apical epithelial cells and basal cells (see Supplementary FIG. 9 for full gating strategy). FIG. 4d shows quantification of flow cytometry for the ratio of basal to Epcam+ epithelial cells recovered from tissue; points represent individual samples; n=6 non-polyp, 7 polyp samples, *t-test, p<0.0005. FIG. 4e shows representative immunofluorescence and quantification for p63-staining cells (basal cell marker) relative to isotype control normalized to 1000 μm2 of epithelial area; quantification of n=5 non-polyp patients from 18 fields of view, and n=8 polyp patients from 36 fields of view; *Mann-Whitney U-test, p<0.0031 with each patient plotted as a point and standard deviation plotted for error bars; scale bar 100 μm. FIG. 4f shows representative histology (5× magnification) and quantification of the glandular area detected in haematoxylin and eosin stained tissue sections from non-polyp or polyp patients; points represent individual patients; n=6 non-polyp, 6 polyp samples, *t-test, p<0.0022. FIG. 4g is a graph depicting principal components analysis (PCA) over epithelial subset-specific genes (Supplementary Table 3) on 27 whole tissue RNA-seq samples from non-polyp (n=9), or polyp (n=18) patients with clusters identified by KNN (n=set to 4; Methods). FIG. 4h is a column-normalized heatmap over epithelial subset-specific genes (Supplementary Table 3) grouped by KNN-clusters from FIG. 4g, color bars disease annotation (non-polyp=green, polyp=purple) and lower bar clusters from FIG. 4g. FIG. 4i is a violin plot of expression contribution to a cell's transcriptome of basal cell genes (effect size 0.457, polyp vs non-polyp; see Methods and Supplementary Table 4) across all recovered epithelial cells in non-polyp and polyp ecosystems; n=6 non-polyp, 6 polyp samples, *t-test p<2.2×10−16.

FIG. 5—Extrinsic pathways converge at the chromatin level in basal cells to intrinsically impair differentiation in vivo. FIG. 5a is a row-normalized heatmap of select differentially expressed genes using bimodal test over single-cells from basal cell clusters 8 and 12; n=6 non-polyp, 6 polyp samples (see Supplementary Table 3 for genes and statistics, *bimodal test, all displayed genes p<1.97×10−39 or less with Bonferroni correction for multiple hypothesis testing based on number of genes tested). FIG. 5b is a violin plot of expression contribution to a cell's transcriptome over IL-4/IL-13 commonly-induced gene signature in respiratory epithelial cells; n=6 non-polyp, 6 polyp samples (see Methods, FIG. 16 for unique genes, and Supplementary Table 4 for gene lists used, all p<1.98×10−15, relative to mean score, with Bonferroni correction for multiple comparisons). FIG. 5c is a violin plots of shared IL-4/IL-13 signature expression contribution to a cell's transcriptome (effect size 1.305, polyp vs. non-polyp) and of the Wnt:Notch target gene proportion (effect size 0.334, polyp vs. non-polyp, NB: axis truncated at −2.5 and 2.5) (see Methods, and Supplementary Table 4 for gene lists used, zero indicates equal scores, Wnt-pos, Notch-neg direction) across all epithelial cells grouped by disease state; n=6 non-polyp, 6 polyp samples, *t-test p<2.2×10−16 for both. FIG. 5d is a set of graphs showing selected genes detected in fibroblasts from single-cell data which correlate with the samples ranked by basal cell frequency detected within each ecosystem; n=6 non-polyp, 6 polyp samples, all genes used: abs(r)>0.7651, p<0.0037. NB: to determine genes correlated in specific cell types (e.g. fibroblasts) with the frequency of basal cells present in a cellular ecosystem, the average log-normalized single-cell count data for each gene was correlated to the rank of samples determined by increasing frequency of basal cells in each ecosystem (8.2% to 19.1% for non-polyp and 27.9% to 70.1% for polyp samples, FIG. 14b). FIG. 5e shows a clustered correlation matrix of genes identified in FIG. 5d in single-cell data from fibroblasts; abs(r)>0.048 is p<0.05 significant based on asymptotic p-values. FIG. 5f is a graph showing pseudotime analysis using diffusion mapping (see Methods) of selected clusters of epithelial cells, colored by cluster; n=3,516 cells (clusters 8/1/4), n=4,064 cells (clusters 12/2/0), and n=6 non-polyp, 6 polyp samples, diffusion map and DC (diffusion coefficients) are calculated over the set of basal and apical marker genes identified in FIG. 1a, see Supplementary Table 3. FIG. 5g is a violin plot of pseudotime component (see Methods) for cells in respective clusters; shading in green is non-polyp distribution and in purple is polyp distribution underlying respective clusters. FIG. 5h is a graph showing Omni-ATAC-seq (see Methods) profiling and HOMER motif enrichment of non-polyp peaks over all peaks as background and polyp peaks over all peaks as background on low-input sorted basal cell populations; n=3 non-polyp and n=7 polyp; all q-value <0.0002 Benjamini corrected. FIG. 5i is a set of bar graphs showing transcription factors significantly differentially expressed in sorted basal cells (FIG. 15 for sort gate) subjected to low-input RNA-seq which overlap with enriched motifs identified in Omni-ATAC-seq profiling; n=4 non-polyp, 8 polyp samples (*p<0.05 or less with Holm-Sidak correction for multiple hypothesis testing), NB: sorted basal cells from same patients profiled through ATAC-seq.

FIG. 6—Basal cells from polyps ex vivo retain transcriptional memory of IL-4/IL-13 exposure and in vivo blockade with an anti-IL-4Ra monoclonal antibody shifts basal cell state. FIG. 6a is a tSNE plot of 16,173 single cells (8,483 non-polyp and 7,690 polyp) from air-liquid interface (ALI) cultures at 21 days of differentiation initiated with basal cells from n=2 non-polyp and n=2 polyp patients colored by cell types identified through marker discovery (ROC test) and scoring based on deconvolution gene lists (Supplementary Table 3 used in FIGS. 4g and h; Methods) representing 1,345 basal, 6,420 secretory, 6,381 hybrid, and 2,027 ciliated cells. FIG. 6b is a set of violin plots for the count-based expression level (log(scaled UMI+1)) for key marker genes using ROC test across cell types identified in FIG. 6a; *AUC 0.943 for KRT5, 0.667 for TP63, 0.644 for LYPD2, <0.55 for SPDEF, <0.55 for KRT8, 0.602 for BPIFA1, 0.813 for PIFO, 0.73 for FOXJ1. FIG. 6c is a set of violin plots for the count-based expression level (log(scaled UMI+1)) for key in vivo secretory genes from FIG. 3e representing select healthy (S100A9, MUC4), non-polyp (PSCA, SCGB1A1), and polyp (CST1, POSTN) upregulated genes using bimodal test within ALI secretory cells between disease states from non-polyp cultures (3,277 cells, n=2 basal cell donors) and polyp cultures (3,143 cells, n=2 basal cell donors); *bimodal test, not significant (n.s.) except MUC4, PSCA and SCGB1A1 significantly greater expressed in polyp relative to non-polyp secretory cells, p<4.04×10−16 Bonferroni correction for multiple hypothesis testing based on number of genes tested. FIG. 6d is a bar graph showing quantification of flow cytometry for the ratio of basal to Epcamhi cells from ALI cultures at 21 days stimulated with IL-13 over the indicated doses; points represent individual biological replicates; n=6 non-polyp, 5 polyp samples for each dose, *2-way ANOVA, n.s. between disease groups at any dose tested, IL-13 dose significant. FIG. 6e is a graph showing basal cells from n=2 non-polyp and n=2 polyp patients were passaged, seeded at passage 5 (e.g. 5 weeks ex vivo) and cultured at confluence in 96 well plate format before addition of media, or increasing doses (ng/mL) of IL-4, IL-13, and IL-4+IL-13 in combination (n=32 samples non-polyp and n=32 samples polyp basal cells over all conditions, each condition run as a biological duplicate, and a technical duplicate therein) before lysis 12 hours after cytokine addition (Methods); bulk RNA-seq and principal components analysis (PCA) over variable genes (Methods) was performed to allow for a data-driven interpretation of the genes induced by IL-4 and/or IL-13 in vitro in basal cells; circled samples denote no cytokine added. Venn diagram overlaps of gene sets identified as upregulated in unstimulated polyp basal cells relative to unstimulated non-polyp cells, induced in polyp basal cells relative to unstimulated polyp basal cells, or induced in non-polyp basal cells relative to unstimulated polyp basal cells; genes considered significantly differentially expressed as p<0.05 with Bonferroni correction for multiple hypothesis testing based on number of genes tested, Supplementary Table 3 for full gene lists. FIG. 6f is a set of graphs showing expression levels for CTNNB1 and CTGF (Log 2 expression value of log-normalized count data) or gene set score for Wnt pathway (Z-Score, see Methods, Supplementary Table 4 for gene set used) in basal cells from non-polyp or polyp individuals across doses of cytokines displayed; 2-way ANOVA p<0.05 for all conditions non-polyp vs polyp except 0.1 ng/mL IL-4 dose for CTGF. FIG. 6g is a tSNE plot of 8,764 single cells from the nasal polyps of an anti-IL-4Ra (dupilumab) treated individual (1 patient, sampled at n=3 timepoints) colored by cell types identified through marker discovery (ROC test) and biological curation of identified clusters (Supplementary Table 3; Methods); tSNE plot colored by timepoint and tissue of origin from polyp pre-dupilumab scraping (5,731 cells), from polyp post-dupilumab scraping (647 cells), and polyp post-dupilumab surgical sample (2,386 cells; see FIG. 19c for tSNE plot colored by clusters identified through SNN, marker discovery (ROC test) and biological curation of identified clusters (Supplementary Table 3; Methods)). FIG. 6h is a row-normalized heatmap for basal cells (200 cells pre-dupilumab and 151 cells post-dupilumab; circled in FIG. 6g) with select genes displayed on y-axis including a core basal signature (ROC-test basal cells vs. rest of cells, AUC>0.68), and then within basal cells a bimodal test used to identify genes affected by dupilumab treatment, and treatment annotations on x-axis; bimodal test, all non-core genes p<2.46×10−5 or less with Bonferroni correction for multiple hypothesis testing based on number of genes tested, see Supplementary Table 3 for full list. FIG. 6i is a row-normalized heatmap for myeloid cells of the top marker genes identified by ROC-test (AUC>0.8) with select genes displayed on y-axis including a core myeloid signature (ROC-test myeloid cells vs. rest of cells), and then genes found to be differentially expressed from FIG. 6h in basal cells, and treatment annotations on x-axis; bimodal test, *asterisk denotes differential genes in both basal cells and myeloid cells pre- vs post-treatment p<0.003 or less with Bonferroni correction for multiple hypothesis testing based on number of genes tested. FIG. 6j is a set of violin plots for basal cells (200 cells pre-dupilumab and 151 cells post-dupilumab, circled in FIG. 6h) for the count-based expression level (log(scaled UMI+1)), except where indicated for gene scores, fraction of transcriptome and z-Score, see Methods, Supplementary Table 4 for gene set used) for key basal cell genes for selected biological processes, or from the baseline upregulated gene set from polyp basal cells in vitro FIG. 6f; differential expression testing for decreased expression post-treatment using bimodal test n.s. unless denoted by * for p<0.00087 or less with Bonferroni correction for multiple hypothesis testing based on number of genes tested, see Supplementary Table 3 for full list; Wnt score Pre vs Post: effect size 0.942 p<2.2×10−16; Basal in vitro score Pre vs Post: effect size 0.822 p<3.897×10−15.

FIG. 7—Consistency of cell capture and identification in scRNA-seq patient cohort. FIG. 7a is a graph showing number of unique molecular identifiers (nUMI) and genes identified, and fraction of reads mapping to mitochondrial or ribosomal genes across recovered cell types. FIG. 7b is a graph showing number of unique molecular identifiers (nUMI) and genes identified, and fraction of reads mapping to mitochondrial or ribosomal genes across patient samples. FIG. 7c is a tSNE plot as in FIG. 1b colored by cell types across all patients and then separated by sample. FIG. 7d is a graph showing the percentage of each cell type recovered within each sample.

FIG. 8—Top marker genes for cell types by scRNA-seq and bulk tissue RNA-seq recovers expected type 2 immune and eosinophilic module. FIG. 8a is a row-normalized heatmap of the top 10 marker genes identified by ROC-test (AUC>0.73 for all) over all cell types (FIG. 1b) with select genes displayed on y-axis and cluster annotations on x-axis (see Supplementary Table 3 for full gene lists). FIG. 8b is a graph showing an overlay of CLC displaying binned count-based expression level (log(scaled UMI+1) amongst myeloid cells (a pathognomonic gene for eosinophils). FIG. 8c is a row-normalized and row- and column-clustered heatmap over the top 100 positively and negatively differentially-expressed genes (50 in each direction) in bulk tissue RNA-seq of 27 samples from non-polyp (n=9) and polyp (n=18) tissue with select genes displayed; all p<9.03×10−5 for genes displayed corrected for multiple comparisons by Benjamini procedure, (see Supplementary Table 3 for full gene list and associated statistics). FIG. 8d is a graph showing the top differentially regulated pathways identified by Ingenuity Pathway Analysis (see Methods) over the top 1,000 differentially expressed genes, as determined by p<0.05 corrected for multiple comparisons by Benjamini procedure, across polyp and non-polyp tissue. FIG. 8e is a graph showing the predicted upstream regulators based on differentially expressed gene modules in polyp tissue relative to non-polyp determined using Ingenuity Pathway Analysis (see Methods).

FIG. 9—Subclustering of myeloid, fibroblast and endothelial cell types. FIG. 9a is a tSNE plot of 811 myeloid cells (n=12 samples non-polyp and polyp), colored by clusters identified through shared nearest neighbor (SNN) analysis (Supplementary Table 3; Methods) from ethmoid sinus tissue; select marker gene overlays displaying count-based (unique molecular identifier (UMI) collapsed) expression level (log(scaled UMI+1)) on a tSNE plot (see Supplementary Table 3 for full gene lists; genes identified via ROC test with AUC 0.689 for S100A8, 0.763 for CD1C, 0.927 for C1QC); a clustered correlation matrix of marker genes identified in single-cell data from myeloid cells; and violin plots for the expression value (log(scaled UMI+1)) of selected markers of myeloid activation state. FIG. 9b is a tSNE plot of 1,724 fibroblasts (n=12 samples non-polyp and polyp), colored by clusters identified through shared nearest neighbor (SNN) analysis (Supplementary Table 3; Methods) from ethmoid sinus tissue; select marker gene overlays displaying count-based (unique molecular identifier (UMI) collapsed) expression level (log(scaled UMI+1)) on a tSNE plot (see Supplementary Table 3 for full gene lists; genes identified via ROC test with AUC 0.691 for CTGF, 0.683 for CXCL12, 0.726 for MYH11); and a clustered correlation matrix of marker genes identified in single-cell data from fibroblasts. NB: Clusters 4 and 5 likely represent doublets with epithelial cells and endothelial cells, respectively. While these clusters were excluded from further formal analyses, it is noted that there may be interesting biology within pairs of cells found to interact more frequently than by chance. FIG. 9c is a tSNE plot of 1,143 endothelial cells (n=12 samples non-polyp and polyp), colored by clusters identified through shared nearest neighbor (SNN) analysis (Supplementary Table 3; Methods) from ethmoid sinus tissue; select marker gene overlays displaying count-based (unique molecular identifier (UMI) collapsed) expression level (log(scaled UMI+1)) on a tSNE plot (see Supplementary Table 3 for full gene lists; genes identified via ROC test with AUC 0.742 for SELE, 0.706 for PODXL, 0.822 for PLAT); and a clustered correlation matrix of marker genes identified in single-cell data from endothelial cells.

FIG. 10—The identities of T cells in type 2 immunity. FIG. 10a is a tSNE plot of re-clustered T cells with select gene overlays displaying binned count-based expression level (log(scaled UMI+1) for Th2A-specific genes (top row) and canonical T cell markers (bottom row). FIG. 10b is a violin plot of five identified T cell clusters scored for expression of T cell receptor complex genes (e.g. TRAC and CD3E, see Methods, Supplementary Table 4). FIG. 10c is a dot plot of inducers and effectors of Type 1 immunity across all cell types (Note: IL17F not detected).

FIG. 11—Mapping type 2 inflammatory mediators within non-polyp or polyp ecosystems. FIG. 11a is a set of dot plots of chemokines and lipid mediators with known roles in type 2 immunity mapped onto cell types divided by non-polyp or polyp disease state, dot size represents fraction of cells within that type expressing, and color intensity binned (log(scaled UMI+1)) gene expression amongst expressing cells (related to FIG. 1d). FIG. 11b is a dot plot of inducers and effectors of type 2 immunity mapped onto cell types divided by non-polyp or polyp disease state, dot size represents fraction of cells within that type expressing, and color intensity binned (log(scaled UMI+1)) gene expression amongst expressing cells (related to FIG. 1e). FIG. 11c is a dot plot of select GWAS risk alleles41 for allergic disease, mapped onto cell types divided by non-polyp or polyp disease state, dot size represents fraction of cells within that type expressing, and color intensity binned (log(scaled UMI+1)) gene expression amongst expressing cells (related to FIG. 1e).

FIG. 12—Relationship of epithelial cell clusters and secretory/glandular distinctions. FIG. 12a is a phylogenetic tree based on the average cell from each cluster of epithelial cell clusters in gene-space. FIG. 12b is a violin plot of expression contribution to a cell's transcriptome of basal cell genes (see Methods and Supplementary Table 4) across all epithelial cells; *t-test each cluster score vs. the average score of all epithelial cells. FIG. 12c is a graph showing canonical correlation analysis (CCA) displaying our cell type annotations for basal and apical cells derived through clustering and biological curation alongside CCA clusters in tSNE space. FIG. 12d is a set of violin plots for the count-based expression level (log(scaled UMI+1)) of selected marker genes for each identified epithelial cell subset. FIG. 12e is a graph showing select overlays on clusters 0 and 4 (differentiating/secretory) and 3 (glandular) displaying binned count-based expression level (log(scaled UMI+1) in tSNE space for canonical goblet (MUC5B, MUC5AC, SPDEF, FOXA3) and secretory (SCGB1A1) genes. FIG. 12f shows a clustered correlation matrix of glandular, goblet, and secretory cell genes; abs(r)>0.038 is p<0.05 significant based on asymptotic p-values.

FIG. 13—Glandular cell subsets and their relationship to apical secretory cells. FIG. 13a is a set of tSNE plots of 5,928 single epithelial cells (n=6 non-polyp samples), and 4,346 single epithelial cells (n=6 polyp samples) colored by clusters identified through (Left) shared nearest neighbor (SNN) analysis and (Right) original biological curation of cell types (Supplementary Table 3; Methods) as illustrated in FIG. 2a. NB: cluster colors in Left panels of each disease are not comparable but curated clusters in Right are, and glandular cells are highlighted for subsetting in next panel. FIG. 13b is a set of violin plots for the count-based expression level (log(scaled UMI+1)) of selected marker genes identified through marker discovery (ROC test) for each subset of glandular cells; n=2,114 total cells with representation of every non-polyp patient in each cluster of cells (e.g. no cluster is unique to one patient) and AUC metric 0.800 for LCN2, 0.736 for SERPINB3, 0.985 for MUC5B, 0.973 for BPIFB2, and 0.908 for PRB1. FIG. 13c is a diagram showing that samples were acquired through the two distinct methods of nasal scraping and ethmoid sinus surgical intervention. This allowed for sampling of left: healthy tissue from inferior turbinate (scraping), middle: of CRS non-polyp tissue from ethmoid sinus (surgery), right: of CRS polyp tissue from ethmoid sinus (surgery), of inferior turbinates of polyp-bearing individuals (scraping), and of polyp tissue accessible for scraping (scraping). Anatomy of the nasal turbinates (healthy and CRS polyp) and ethmoid sinus (CRS non-polyp and CRS polyp) where samples were acquired is displayed below, highlighting the depth of cells recovered from each site related to FIG. 3. Healthy tissue is annotated with basal and apical cell types, including sub-mucosal glands.

FIG. 14—Changes in cellular composition between non-polyp and polyp sinus tissue. FIG. 14a is a graph showing the frequency of each cell type recovered amongst all cells within each patient sample (n=6 non-polyp, 6 polyp) grouped by disease state; *t-test p<0.05 for Apical, Glandular, Ciliated, Plasma Cell, T Cell, Myeloid and Mast Cell with Holm-Sidak correction for multiple comparisons. FIG. 14b is a graph showing the frequency of basal cells amongst epithelial cells captured in scRNA-seq data displayed for each sample and colored by non-polyp or polyp designation. FIG. 14c is a set of tSNE plots with each patient's cells clustered independently over a common list of most variable genes identified from all epithelial cells and with clustering parameters set constant to 12 principal components and resolution set to 1.4. FIG. 14d is a graph showing Simpson's index of diversity over epithelial cell clusters identified in FIG. 14c, an indication of the total richness present within an ecosystem, calculated for each patient; points represent individual patients, *t-test p<0.0384. FIG. 14e is graph showing correlation of Simpson's index of diversity calculated over epithelial cells against the ranked order of samples based on clinical pathological evaluation; r=0.6824, p<0.009. FIG. 14f is a graph showing Simpson's index of diversity over stromal and immune cell types and total cells, an indication of the total richness present within an ecosystem, calculated for each sample; points represent individual samples, *t-test p<0.0015 stromal and immune, p<0.0145 total cells, non-polp vs. polyp.

FIG. 15—Flow cytometric gating strategy for quantification and isolation of basal cells and validation of basal cell hyperplasia relative to healthy tissue. FIG. 15a is a tSNE plot of 10,274 epithelial cells reproduced from FIG. 2a, colored by clusters identified through SNN, with adjacent color bars representing related cell clusters, and overlays displaying binned count-based expression level (log(scaled UMI+1) of selected genes used to negatively (CD45, EPCAM, THY1) and positively (NGFR, ITGA6, PDPN) identify basal cells. FIG. 15b is a graph showing full gating strategy for quantification and isolation of basal cells from non-polyp and polyp tissue, (related to FIG. 4c,d). FIG. 15c is a graph showing basal cell fraction of transcripts from bulk tissue RNA-seq data of our own data set (related to FIG. 4g,h) and two GEO data sets (NB: analysis is done per sample and as such no comparisons across the data sets are made) containing healthy and healthy/polyp nasal mucosa biopsies; *t-test p<0.05. FIG. 15d is graph showing secretory cell fraction of transcripts from bulk tissue RNA-seq data of our own data set (related to FIG. 4g,h) and two GEO data sets (NB: analysis is done per sample and as such no comparisons across the data sets are made) containing healthy and healthy/polyp nasal mucosa biopsies; *t-test p<0.05.

FIG. 16—Epithelial cytokine signatures from CRS sinus tissue demonstrate Type 2 inflammatory pattern and differential expression within myeloid and endothelial cells by polyp status. FIG. 16a is a set of violin plots of IL-4 or IL-13 uniquely induced gene signatures in respiratory epithelial cell clusters or grouped by disease state presented as expression contribution to a cell's transcriptome (see Methods, FIG. 5b for shared genes, and Supplementary Table 4); *t-test p<2.2×10−16, 0.305 effect size IL-4 polyp vs. non-polyp and −0.448 effect size IL13 polyp vs non-polyp. FIG. 16b is a set of violin plots of IFNa or IFNy induced gene signatures in respiratory epithelial cell clusters or grouped by disease state presented as expression contribution to a cell's transcriptome (see Methods, and Supplementary Table 4); *t-test p<5.1×10−16 for both, −0.156 effect size IFNa polyp vs. non-polyp and 0.161 effect size IFNy polyp vs non-polyp. FIG. 16c is a row-normalized heatmap for myeloid cells from ethmoid sinus with select genes displayed on y-axis including a core myeloid signature (ROC-test myeloid cells vs. rest of cells, AUC>0.8), and genes differentially expressed (bimodal test) by disease state, with disease state annotations on x-axis; bimodal test, all non-core genes p<0.0002 or less with Bonferroni correction for multiple hypothesis testing based on number of genes tested. FIG. 16d is a row-normalized heatmap for endothelial cells from ethmoid sinus with select genes displayed on y-axis including a core basal signature (ROC-test endothelial cells vs. rest of cells, AUC>0.75), and genes differentially expressed (bimodal test) by disease state, with disease state annotations on x-axis; bimodal test, all non-core genes p<2.43×10−6 or less with Bonferroni correction for multiple hypothesis testing based on number of genes tested.

FIG. 17—Pseudotime analysis on basal and differentiating/secretory cell clusters. FIG. 17a is a graph showing pseudotime analysis using diffusion mapping (see Methods) of selected clusters of epithelial cells, here displaying diffusion pseudotime (related to FIG. 5f); n=3,516 cells (clusters 8/1/4), n=4,064 cells (clusters 12/2/0), and n=6 non-polyp, 6 polyp samples, diffusion map and DC (diffusion coefficients) are calculated over the set of basal and apical marker genes identified in FIG. 1a, see Supplementary Table 3. FIG. 17b is a graph showing the top 60 negatively correlated genes expressed in non-polyp cells with pseudotime trajectory and Pearson correlation values for genes in polyp cells also displayed; differential correlation coefficient analysis using Fisher's Z-statistic, accounting for number of cells in each group (specific genes highlighted all >2 Z, full results including Bonferroni corrected p values in Supplementary Table 3).

FIG. 18—Transcriptional motif enrichments in non-polyp and polyp basal cells. FIG. 18a is a set of correlation matrices (row and column clustered) of the normalized read counts per sample in motif associated-peaks for non-polyp or polyp samples; Pearson correlation, n=3 non-polyp, n=7 polyp. FIG. 18b is a column-normalized heatmap (row and column clustered) for the fraction of peaks with a motif corresponding to accessibility of the respective transcription factor displayed by patient; n=3 non-polyp, n=7 polyp. FIG. 18c is a graph showing IGV tracks for ATF3 and KLF5 based on peaks detected and averaged by non-polyp and polyp samples from ATAC-seq profiling. FIG. 18d is a graph showing IGV tracks for S100A9 and MUC4 based on peaks detected and averaged by non-polyp and polyp samples from ATAC-seq profiling.

FIG. 19—Polyp basal cells can differentiate to secretory cells in vitro and in vivo blockade with an anti-IL-4Ra monoclonal antibody shifts secretory cell state towards healthy-associated genes. FIG. 19a is a row-normalized heatmap for ALI-secretory cells (subsampled to 300 cells per donor) as in FIG. 3e of the top in vivo secretory marker genes identified by ROC-test (AUC>0.662) with select genes displayed on y-axis including a core secretory signature (ROC-test secretory cells vs. rest of cells), and then within secretory cells a ROC-test used to identify marker genes within each disease/location category, and basal-cell derived annotations on x-axis (see Supplementary Table 3 for full gene lists, all AUC>0.65 for markers displayed in FIG. 3e). FIG. 19b is a row-normalized and row-clustered heatmap for in vitro stimulated basal cells (n=32 samples non-polyp and n=32 samples polyp basal cells over all conditions, each condition run as a biological duplicate, and a technical duplicate therein; Methods) as in FIG. 6e over the in vivo pseudotime differentiation trajectory genes (FIG. 5f, see Supplementary Table 3 for gene list and highlighted modules) with select genes displayed on y-axis. FIG. 19c is a set of tSNE plots including left: tSNE plot of 8,764 single cells (related to FIG. 6g) from the nasal polyps of an anti-IL-4Ra (dupilumab) treated individual (1 patient, sampled at n=3 timepoints) colored by clusters identified through shared nearest neighbor (SNN) analysis (Supplementary Table 3; Methods); middle: tSNE plot colored by timepoint and tissue of origin from polyp pre-dupilumab scraping (5,731 cells), from polyp post-dupilumab scraping (647 cells), and polyp post-dupilumab surgical sample (2,386 cells); and right: tSNE plot colored by cell types identified through marker discovery (ROC test) and biological curation of identified clusters (Supplementary Table 3; Methods)). FIG. 19d is a graph showing select cell-type specific score overlays for cell types indicated in original core data set (see Supplementary Table 3 for full gene list). FIG. 19e is a tSNE plot of 4,486 single cells (related to FIG. 3d, and FIG. 6g) from the inferior turbinate or nasal polyps of an anti-IL-4Ra (dupilumab) treated individual (n=4 samples) colored by timepoint and tissue of origin from inferior turbinate pre-dupilumab scraping (643 cells), from inferior turbinate post-dupilumab scraping (1,596 cells), polyp pre-dupilumab scraping (1,600 cells), and polyp post-dupilumab scraping (647 cells); and tSNE plot colored by cell types identified through marker discovery (ROC test) and biological curation of identified clusters (Supplementary Table 3; Methods)); black outline indicates cells considered in FIG. 19g. FIG. 19f is a graph showing select deconvolution score overlays for cell types indicated in original core data set (see Supplementary Table 3 for full gene list). FIG. 19g is a violin plot for the gene set score over Wnt pathway (z-score) and expression contribution to a cell's transcriptome over IFNα and IL-4/IL-13 commonly-induced gene signature in secretory cells grouped as in (e) and sub-sampled to a maximum of 150 cells from each disease/location category from inferior turbinate pre-dupilumab scraping (150 cells), from inferior turbinate post-dupilumab scraping (23 cells), polyp pre-dupilumab scraping (150 cells), and polyp post-dupilumab scraping (38 cells); see Methods, Supplementary Table 4 for gene lists used; Wnt score Pre vs Post Polyp Tissue: effect size 1.02, p<1.091×10−14; Wnt score Pre vs Post Inferior Turbinate Tissue: effect size −0.17, p=0.3706; IL4/IL13 score Pre vs Post Polyp Tissue: effect size 1.17, p<2.2×10−16; IL4/IL13 score Pre vs Post Inferior Turbinate Tissue: effect size −0.17, p=0.163; IFNα score Pre vs Post Polyp Tissue: effect size −1.25, p<4.254×10−05; IFNα score Pre vs Post Inferior Turbinate Tissue: effect size −0.304, p=0.2766; differential expression testing for decreased expression post-treatment using bimodal test denoted by * and p<7.81×10−06 or less between pre- and post-treated polyp. FIG. 19h is a set of violin plots of secretory cells grouped as in (e) and sub-sampled to a maximum of 150 cells from each disease/location category from inferior turbinate pre-dupilumab scraping (150 cells), inferior turbinate post-dupilumab scraping (23 cells), polyp pre-dupilumab scraping (150 cells), and polyp post-dupilumab scraping (38 cells) for the count-based expression level (log(scaled UMI+1)) and for secretory cell genes from the gene set used in FIG. 3e affected by treatment within anatomical regions indicated by heading; differential expression testing for decreased expression post-treatment using bimodal test n.s. unless denoted by *, all p<6.36×10−5 or less except KLF5 (p<0.0033) and FOSB (p<0.0053) with Bonferroni correction for multiple hypothesis testing based on number of genes tested, see Supplementary Table 3 for all genes tested.

FIG. 20—is a graph showing in vivo basal polyp vs non-polyp gene expression differences (FIG. 5a, Table 15) overlapped with the Pre- vs Post-dupilumab treatment (FIG. 6H and Table 18).

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein a “barrier cell” or “barrier tissues” refers generally to various epithelial tissues of the body such, but not limited to, those that line the respiratory system, digestive system, urinary system, and reproductive system as well as cutaneous systems. The epithelial barrier may vary in composition between tissues but is composed of basal and apical components, or crypt/villus components in the case of intestine

Within the present specification, the terms “differentiation”, “differentiating” or derivatives thereof, denote the process by which an unspecialised or relatively less specialised cell becomes relatively more specialised. In the context of cell ontogeny, the adjective “differentiated” is a relative term. Hence, a “differentiated cell” is a cell that has progressed further down a certain developmental pathway than the cell it is being compared with. The differentiated cell may, for example, be a terminally differentiated cell, i.e., a fully specialised cell capable of taking up specialised functions in various tissues or organs of an organism, which may but need not be post-mitotic; or the differentiated cell may itself be a progenitor cell within a particular differentiation lineage which can further proliferate and/or differentiate.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide a cell atlas of barrier tissues from healthy and diseased subject. The present invention discloses novel markers for cell types. Moreover, genes associated with disease, including type 2 inflammatory (T2I) response are identified. T2I plays a dual role in regulating homeostatic processes, such as metabolism¹², and promoting inflammatory defense mechanisms in response to parasites, venoms, allergens, and toxins¹³. T2I is characterized by sensor cells (epithelia, macrophages, dendritic cells, mast cells) producing first-order cytokines (TSLP, IL-25, IL-33) that cause release of second-order cytokines (IL-4, IL-5, IL-13, AREG) from lymphocytes^{5,14}. This, in turn, helps recruit effector cells (eosinophils, basophils, mast cells, monocytes), and induces epithelial remodeling, such as goblet cell hyperplasia¹⁵, to restore tissue integrity^{5,14}. Accumulating evidence suggests that immune modules, which are productively activated in acute settings to restore homeostasis, may reach an aberrant set point during chronic inflammatory diseases such as CRS^{16,17}. For example, cytokine modules may become self-reinforcing in T2I leading to substantial alterations in gross tissue architecture¹⁸ and the reduced epithelial barrier integrity characteristic of severe clinical presentations, such as polyps^{19,20}Whether and how human cellular tissue ecosystems shift in composition and function during chronic T2I disease remains unknown. Accordingly, diseases and disorders that may be detected include allergic asthma, therapy resistant-asthma, steroid-resistant severe allergic airway inflammation, systemic steroid-dependent severe eosinophilic asthma, chronic rhino-sinusitis (CRS), atopic dermatitis, food allergies, persistence of chronic airway inflammation, and primary eosinophilic gastrointestinal disorders (EGIDs), including but not limited to eosinophilic esophagitis (EoE), eosinophilic gastritis, eosinophilic gastroenteritis, and eosinophilic colitis (see, e.g., Van Rijt et al., Type 2 innate lymphoid cells: at the cross-roads in allergic asthma, Seminars in Immunopathology July 2016, Volume 38, Issue 4, pp 483-496; Rivas et al., IL-4 production by group 2 innate lymphoid cells promotes food allergy by blocking regulatory T-cell function, J Allergy Clin Immunol. 2016 September; 138(3):801-811.e9; and Morita, Hideaki et al. Innate lymphoid cells in allergic and nonallergic inflammation, Journal of Allergy and Clinical Immunology, Volume 138, Issue 5, 1253-1264). In non-mucosal tissues, this would also include atopic dermatitis/eczema.

In certain embodiments, diseases are treated using the embodiments disclosed herein, including any disease or disorder involving T2I which underlie allergic inflammatory disease. Example diseases and disorders include, but are not limited to, allergic rhinitis, chronic rhinosinusitis without nasal polyps, chronic rhinosinusitis with nasal polyps, asthma, eosinophilic esophagitis, fibrosis of respiratory tissue, ulcerative colitis, some types of food allergy and food sensitivity. In non-mucosal tissues, example diseases and disorders involving T2I include, but are not limited to, atopic dermatitis and eczema.

The invention provides for diagnostic assays based on gene markers and cell composition, as well as therapeutic targets for controlling differentiation, proliferation, maintenance and/or function of the cell types disclosed herein. In addition, novel cell types and methods of quantitating, detecting and isolating the cell types are disclosed.

The invention further provides method for modulating cellular interactions within cellular ensembles. A “cellular ensemble” as used herein comprises two or more cell types. The two or more cell types may exist in a two-dimensional in vitro or ex vivo tissue culture or a three-dimensional in vitro or ex vivo tissue culture. The cellular ensemble may also comprise a tissue on a chip, an organoid, or in vivo cells within a particular tissue of interest, such as a diseased tissue, or within a particular signaling microenvironment.

A tissue on a chip, tissue chips, organ on a chip, or organ chips refer to devices designed as accurate models of the structure and function of human organs, such as those of the lung, liver, and heart. Example tissue chips are described Brugmann and Wells, Stem Cell Research & Therapy 2013 4(Suppl1):S1; Guo et al., Stem Cell Research & Therapy 2013 4(Suppl1)S2, Hogberg et al. Stem Cell Research & Therapy 2013 4(Suple1):S4.

Organoids are three-dimensional tissue structures, generated from pluripotent stem cells which self-organize and recapitulate complex aspects of their organ counter parts. Nature Cell Biology 20, 633 (2018).

A signaling microenvironment may be within a specific body compartment, or within a specific area of a tissue or organ wherein extracellular signaling from one or more cell types in the microenvironment results in a change in one or more cells states of one or more other cells within the same signaling microenvironment. Extracellular signaling may be receptor-ligand mediated, cytokine/chemokine mediated, or metabolically mediated. Metabolically mediated extracellular signaling may comprise changes in nutrient reflux, changes in cellular byproducts, or a consequence of an environmental perturbation such as a change in pH, temperature, oxygen concentration, carbon dioxide concentration, or other nutrient concentration.

In certain example embodiment, using Seq-Well for massively parallel scRNA-seq of surgical resections from individuals without (non-polyp) or with polyps, CRS was used as a window into the ecosystem of human mucosal tissues to map the location of Type 2 genes to specific cell types. Across non-polyp and polyp cellular ecosystems, an increase in the basal cell state and accompanied loss of overall barrier diversity with a significant decrease in globlet, secretory, and ciliated cells was observed.

### Biormarker and Signatures

The invention further relates to various biomarkers for quantitating, detecting or isolating gut cell subpopulations. The populations may be detected by detecting one or more biomarkers in a sample. Biomarkers of the present invention as listed in Table 1-10 defined below. The biomarkers may be selected from Table 1, Table 2, Table 3, Table 4, Table 5, Table 6, Table 7, Table 8, Table 9, Table 10, Table 11, Table 12, Table 13, Table 14, Table 15 or a combination thereof.

The term “biomarker” is widespread in the art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism, e.g., in a biological sample of a subject) is predictive or informative with respect to one or more aspects of the tested object's phenotype and/or genotype. The terms “marker,” “biomarker,” “expression product,” may be used interchangeably throughout this specification. Biomarkers as intended herein may be nucleic acid-based or peptide-, polypeptide- and/or protein-based. For example, a marker may be comprised of peptide(s), polypeptide(s) and/or protein(s) encoded by a given gene, or of detectable portions thereof. Further, whereas the term “nucleic acid” generally encompasses DNA, RNA and DNA/RNA hybrid molecules, in the context of markers the term may typically refer to heterogeneous nuclear RNA (hnRNA), pre-mRNA, messenger RNA (mRNA), or complementary DNA (cDNA), or detectable portions thereof. Such nucleic acid species are particularly useful as markers, since they contain qualitative and/or quantitative information about the expression of the gene. Particularly preferably, a nucleic acid-based marker may encompass mRNA of a given gene, or cDNA made of the mRNA, or detectable portions thereof. Any such nucleic acid(s), peptide(s), polypeptide(s) and/or protein(s) encoded by or produced from a given gene are encompassed by the term “gene product(s)”.

Preferably, markers as intended herein may be extracellular or cell surface markers, as methods to measure extracellular or cell surface marker(s) need not disturb the integrity of the cell membrane and may not require fixation/permeabilization of the cells.

Unless otherwise apparent from the context, reference herein to any marker, such as a peptide, polypeptide, protein, or nucleic acid, may generally also encompass modified forms of said marker, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “polypeptide” as used throughout this specification generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The reference to any marker, including any peptide, polypeptide, protein, or nucleic acid, corresponds to the marker commonly known under the respective designations in the art. The terms encompass such markers of any organism where found, and particularly of animals, preferably warm-blooded animals, more preferably vertebrates, yet more preferably mammals, including humans and non-human mammals, still more preferably of humans.

The terms particularly encompass such markers, including any peptides, polypeptides, proteins, or nucleic acids, with a native sequence, i.e., ones of which the primary sequence is the same as that of the markers found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of markers are intended herein. Accordingly, all sequences of markers found in or derived from nature are considered “native”. The terms encompass the markers when forming a part of a living organism, organ, tissue or cell, when forming a part of a biological sample, as well as when at least partly isolated from such sources. The terms also encompass markers when produced by recombinant or synthetic means.

In certain embodiments, markers, including any peptides, polypeptides, proteins, or nucleic acids, may be human, i.e., their primary sequence may be the same as a corresponding primary sequence of or present in a naturally occurring human markers. Hence, the qualifier “human” in this connection relates to the primary sequence of the respective markers, rather than to their origin or source. For example, such markers may be present in or isolated from samples of human subjects or may be obtained by other means (e.g., by recombinant expression, cell-free transcription or translation, or non-biological nucleic acid or peptide synthesis).

The reference herein to any marker, including any peptide, polypeptide, protein, or nucleic acid, also encompasses fragments thereof. Hence, the reference herein to measuring (or measuring the quantity of) any one marker may encompass measuring the marker and/or measuring one or more fragments thereof.

For example, any marker and/or one or more fragments thereof may be measured collectively, such that the measured quantity corresponds to the sum amounts of the collectively measured species. In another example, any marker and/or one or more fragments thereof may be measured each individually. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The term “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example 2 50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

The term “fragment” as used throughout this specification with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

Cells such as epithelia cells, epithelial stem cells, and immune cells as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells.

The present invention is also directed to signatures and uses thereof. As used herein a “signature” may encompass any gene or genes, protein or proteins, or epigenetic element(s) whose expression profile or whose occurrence is associated with a specific cell type, subtype, or cell state of a specific cell type or subtype within a population of cells (e.g., tumor infiltrating lymphocytes). In certain embodiments, the expression of the CD8⁺ TIL signatures are dependent on epigenetic modification of the genes or regulatory elements associated with the genes. Thus, in certain embodiments, use of signature genes includes epigenetic modifications that may be detected or modulated. For ease of discussion, when discussing gene expression, any gene or genes, protein or proteins, or epigenetic element(s) may be substituted. Reference to a gene name throughout the specification encompasses the human gene, mouse gene and all other orthologues as known in the art in other organisms. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations. Generally, where a decrease of a gene or gene expression product is referred to, this means that the gene or gene expression product is repressed, downregulated, knocked-out, inhibited, antagonized, deactivated or other terms common in the art. Similarly, where an increase of a gene or gene expression product is referred to, this means that the gene or gene expression product is enhanced, upregulated, knocked-in, agonized, activated or other terms common in the art. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population state if it is upregulated or only present, detected or detectable in that particular cell or cell (sub)population state (e.g., disease or healthy), or alternatively is downregulated or only absent, or undetectable in that particular cell or cell (sub)population state. In this context, a signature consists of one or more differentially expressed genes/proteins or differential epigenetic elements when comparing different cells or cell (sub)populations, including comparing different gut cell or gut cell (sub)populations, as well as comparing gut cell or gut cell (sub)populations with healthy or disease (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population or subpopulation level, refer to genes that are differentially expressed in all or substantially all cells of the population or subpopulation (such as at least 80%, preferably at least 90%, such as at least 95% of the individual cells). This allows one to define a particular subpopulation of immune cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferably it is meant: induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In certain example embodiments, the signature genes may be used to deconvolute the network of cells present in a tumor based on comparing them to data from bulk analysis of a tissue sample, including barrier tissue samples. In certain example embodiments, the presence of specific immune cells and immune cell subtypes may be indicative of tumor growth, invasiveness and/or resistance to treatment. In one example embodiment, detection of one or more signature genes may indicate the presence of a particular cell type or cell types. In certain example embodiments, the presence of immune cell types within a tumor may indicate that the tumor will be sensitive to a treatment (e.g., checkpoint blockade therapy). In one embodiment, the signature genes of the present invention are applied to bulk sequencing data from a tumor sample obtained from a subject, such that information relating to disease outcome and personalized treatments is determined.

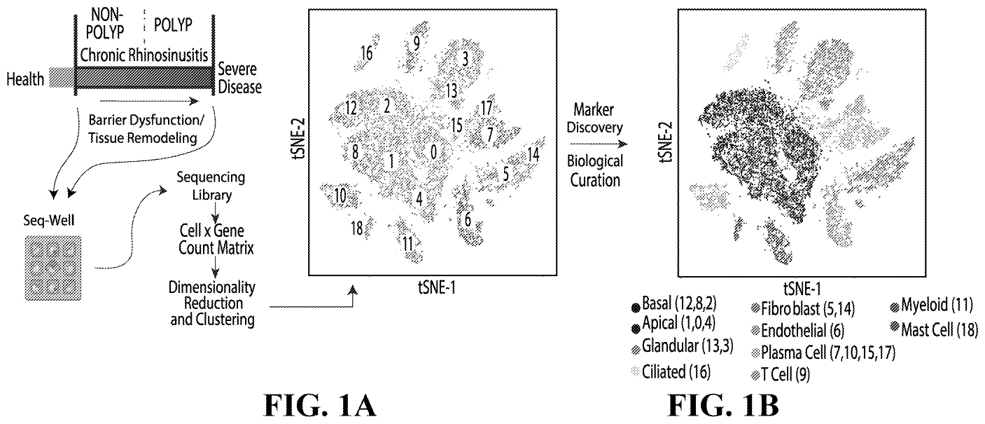

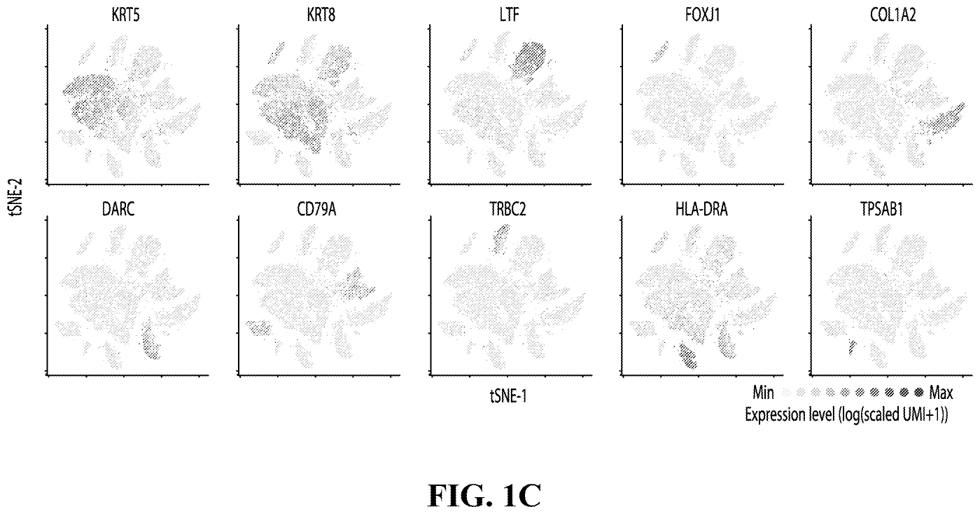

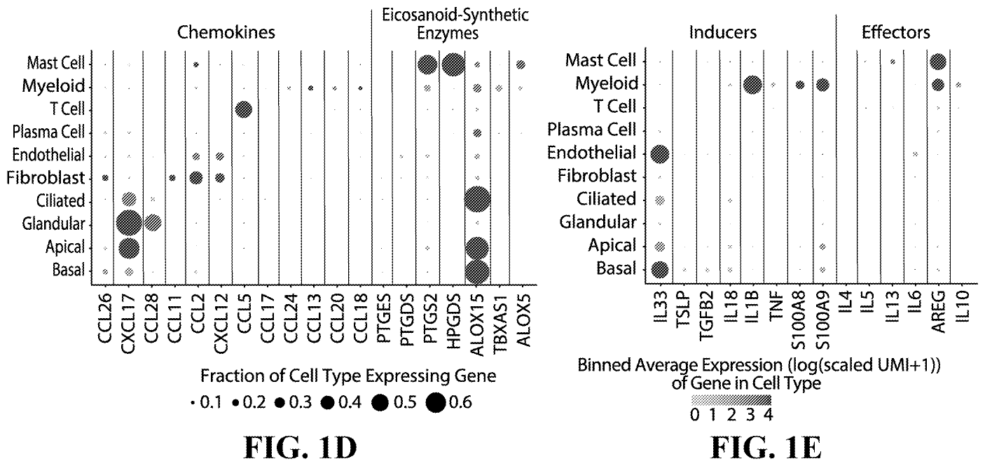

In certain example embodiments, the biomarker and/or target of the barrier cell modulating agent is one or more of the genes or gene expression products listed in Table 1. The cluster numbers in Table 1 refer to the clusters and cell types as labeled in FIG. 1. The genes identified in Table 1 and subsequent tables were determined using scRNA-seq analysis of a combination of healthy control, chronic rhinosinusitis without polyps, chronic rhinosinusitis with polyps, and AERD with nasal polyps which generally refers to patients that are “allergic to life” but also includes a specific subset of patients sensitive to aspirin as well.

TABLE 1

| | myAUC | avg_diff | power | pct. 1 | pct. 2 | cluster | gene |
|---|---|---|---|---|---|---|---|
| SERPINB3 | 0.928 | 1.895204266 | 0.856 | 0.984 | 0.426 | 0 | SERPINB3 |
| CST1 | 0.88 | 2.981620892 | 0.76 | 0.811 | 0.109 | 0 | CST1 |
| ALOX15 | 0.801 | 1.199750674 | 0.602 | 0.834 | 0.358 | 0 | ALOX15 |
| SERPINB4 | 0.795 | 1.560342847 | 0.59 | 0.732 | 0.201 | 0 | SERPINB4 |
| S100A6 | 0.788 | 0.75610193 | 0.576 | 0.983 | 0.805 | 0 | S100A6 |
| KRT19 | 0.776 | 0.843476072 | 0.552 | 0.939 | 0.566 | 0 | KRT19 |
| CST4 | 0.766 | 2.676843661 | 0.532 | 0.564 | 0.047 | 0 | CST4 |
| CLDN4 | 0.765 | 1.187429829 | 0.53 | 0.794 | 0.396 | 0 | CLDN4 |
| GSTP1 | 0.739 | 0.774377124 | 0.478 | 0.861 | 0.607 | 0 | GSTP1 |
| IGFBP3 | 0.738 | 1.544300101 | 0.476 | 0.597 | 0.156 | 0 | IGFBP3 |
| ANXA1 | 0.738 | 0.746609127 | 0.476 | 0.907 | 0.672 | 0 | ANXA1 |
| CD9 | 0.736 | 0.805603012 | 0.472 | 0.841 | 0.573 | 0 | CD9 |
| TACSTD2 | 0.735 | 0.838979307 | 0.47 | 0.824 | 0.488 | 0 | TACSTD2 |
| ANXA2 | 0.73 | 0.770126459 | 0.46 | 0.835 | 0.569 | 0 | ANXA2 |
| AGR2 | 0.723 | 0.81713425 | 0.446 | 0.77 | 0.408 | 0 | AGR2 |
| TSPAN1 | 0.721 | 1.133172415 | 0.442 | 0.624 | 0.226 | 0 | TSPAN1 |
| WFDC2 | 0.719 | 0.701974249 | 0.438 | 0.786 | 0.428 | 0 | WFDC2 |
| KRT18 | 0.715 | 0.928256925 | 0.43 | 0.692 | 0.352 | 0 | KRT18 |
| VMO1 | 0.71 | 1.07600728 | 0.42 | 0.626 | 0.257 | 0 | VMO1 |
| MT1X | 0.707 | 1.12012663 | 0.414 | 0.648 | 0.292 | 0 | MT1X |
| HS3ST1 | 0.706 | 1.162439487 | 0.412 | 0.557 | 0.183 | 0 | HS3ST1 |
| ELF3 | 0.704 | 0.806822371 | 0.408 | 0.714 | 0.375 | 0 | ELF3 |
| PTHLH | 0.703 | 1.274462329 | 0.406 | 0.516 | 0.123 | 0 | PTHLH |
| LGALS3 | 0.703 | 0.689748346 | 0.406 | 0.834 | 0.593 | 0 | LGALS3 |
| TFF3 | 0.696 | 2.140728317 | 0.392 | 0.569 | 0.242 | 0 | TFF3 |
| S100P | 0.692 | 1.268636795 | 0.384 | 0.524 | 0.17 | 0 | S100P |
| KRT8 | 0.686 | 0.769114727 | 0.372 | 0.67 | 0.355 | 0 | KRT8 |
| SLC6A14 | 0.685 | 1.320732692 | 0.37 | 0.466 | 0.119 | 0 | SLC6A14 |
| CST2 | 0.684 | 1.888685867 | 0.368 | 0.382 | 0.016 | 0 | CST2 |
| HSPB1 | 0.684 | 0.734766711 | 0.368 | 0.719 | 0.448 | 0 | HSPB1 |
| FXYD3 | 0.682 | 0.654302559 | 0.364 | 0.763 | 0.505 | 0 | FXYD3 |
| TXN | 0.68 | 0.771956478 | 0.36 | 0.708 | 0.484 | 0 | TXN |
| CSTB | 0.678 | 0.922414544 | 0.356 | 0.591 | 0.306 | 0 | CSTB |
| SAT1 | 0.672 | 0.56530398 | 0.344 | 0.84 | 0.63 | 0 | SAT1 |
| GSN | 0.668 | 1.03303155 | 0.336 | 0.525 | 0.233 | 0 | GSN |
| EZR | 0.664 | 0.57481562 | 0.328 | 0.744 | 0.508 | 0 | EZR |
| ACTG1 | 0.664 | 0.493900071 | 0.328 | 0.855 | 0.674 | 0 | ACTG1 |
| CXCL17 | 0.663 | 0.710513177 | 0.326 | 0.598 | 0.3 | 0 | CXCL17 |
| MTRNR2L1 | 0.663 | 0.439000514 | 0.326 | 0.94 | 0.816 | 0 | MTRNR2L1 |
| PRSS23 | 0.662 | 0.55112375 | 0.324 | 0.724 | 0.444 | 0 | PRSS23 |
| GABRP | 0.659 | 0.97270072 | 0.318 | 0.448 | 0.151 | 0 | GABRP |
| GAPDH | 0.657 | 0.522179298 | 0.314 | 0.789 | 0.637 | 0 | GAPDH |
| CTD-2319I12.1 | 0.656 | 1.2435865 | 0.312 | 0.432 | 0.143 | 0 | CTD-2319I12.1 |
| CDH26 | 0.656 | 1.068055677 | 0.312 | 0.377 | 0.07 | 0 | CDH26 |
| EGLN3 | 0.651 | 1.210168681 | 0.302 | 0.361 | 0.066 | 0 | EGLN3 |
| KRT191 | 0.8 | 0.955061228 | 0.6 | 0.947 | 0.566 | 1 | KRT19 |
| EPAS1 | 0.787 | 1.254246142 | 0.574 | 0.778 | 0.306 | 1 | EPAS1 |
| AQP3 | 0.783 | 1.132080826 | 0.566 | 0.846 | 0.431 | 1 | AQP3 |
| PERP | 0.74 | 0.825325036 | 0.48 | 0.831 | 0.471 | 1 | PERP |
| ANXA11 | 0.729 | 0.74665638 | 0.458 | 0.919 | 0.672 | 1 | ANXA1 |
| KRT81 | 0.714 | 0.89313211 | 0.428 | 0.703 | 0.352 | 1 | KRT8 |
| HSPB11 | 0.714 | 0.840671464 | 0.428 | 0.757 | 0.445 | 1 | HSPB1 |
| UGT2A2 | 0.71 | 1.050395315 | 0.42 | 0.605 | 0.215 | 1 | UGT2A2 |
| PRSS231 | 0.705 | 0.687478271 | 0.41 | 0.791 | 0.439 | 1 | PRSS23 |
| HES1 | 0.703 | 0.999251311 | 0.406 | 0.669 | 0.34 | 1 | HES1 |
| SERPINB31 | 0.702 | 0.632753762 | 0.404 | 0.792 | 0.445 | 1 | SERPINB3 |
| TACSTD21 | 0.701 | 0.61216187 | 0.402 | 0.814 | 0.49 | 1 | TACSTD2 |
| CLDN41 | 0.695 | 0.793489732 | 0.39 | 0.718 | 0.404 | 1 | CLDN4 |
| ELF31 | 0.695 | 0.766397154 | 0.39 | 0.709 | 0.376 | 1 | ELF3 |
| F3 | 0.695 | 0.670905576 | 0.39 | 0.749 | 0.4 | 1 | F3 |
| DUSP1 | 0.686 | 0.613097358 | 0.372 | 0.871 | 0.714 | 1 | DUSP1 |
| MT1X1 | 0.685 | 0.772754974 | 0.37 | 0.627 | 0.295 | 1 | MT1X |
| EGR1 | 0.681 | 0.543249208 | 0.362 | 0.851 | 0.613 | 1 | EGR1 |
| SAT11 | 0.68 | 0.565737643 | 0.36 | 0.852 | 0.629 | 1 | SAT1 |
| ALDH3A1 | 0.677 | 0.949174564 | 0.354 | 0.559 | 0.232 | 1 | ALDH3A1 |
| ZFP36L1 | 0.676 | 0.613988221 | 0.352 | 0.759 | 0.497 | 1 | ZFP36L1 |
| FOS | 0.676 | 0.375428846 | 0.352 | 0.976 | 0.88 | 1 | FOS |
| ALDH1A1 | 0.67 | 0.724295692 | 0.34 | 0.634 | 0.339 | 1 | ALDH1A1 |
| ID1 | 0.665 | 0.59909338 | 0.33 | 0.707 | 0.446 | 1 | ID1 |
| GLUL | 0.664 | 0.611680589 | 0.328 | 0.703 | 0.441 | 1 | GLUL |
| CD91 | 0.664 | 0.445212559 | 0.328 | 0.822 | 0.575 | 1 | CD9 |
| S100A61 | 0.663 | 0.389345358 | 0.326 | 0.947 | 0.808 | 1 | S100A6 |
| PRDX1 | 0.661 | 0.56708492 | 0.322 | 0.73 | 0.504 | 1 | PRDX1 |
| KLF5 | 0.658 | 0.749421349 | 0.316 | 0.541 | 0.259 | 1 | KLF5 |
| AGR21 | 0.654 | 0.2502275 | 0.308 | 0.721 | 0.414 | 1 | AGR2 |
| ATF3 | 0.652 | 0.552872104 | 0.304 | 0.713 | 0.473 | 1 | ATF3 |
| CTSB | 0.652 | 0.502881769 | 0.304 | 0.696 | 0.467 | 1 | CTSB |
| KRT181 | 0.651 | 0.615262302 | 0.302 | 0.63 | 0.359 | 1 | KRT18 |
| POSTN1 | 0.814 | 1.228799705 | 0.628 | 0.882 | 0.413 | 2 | POSTN |
| S100A2 | 0.792 | 1.296822774 | 0.584 | 0.803 | 0.346 | 2 | S100A2 |
| KRT5 | 0.726 | 1.187772267 | 0.452 | 0.632 | 0.236 | 2 | KRT5 |
| KRT15 | 0.724 | 1.462567895 | 0.448 | 0.588 | 0.201 | 2 | KRT15 |
| ALOX151 | 0.707 | 0.985262628 | 0.414 | 0.682 | 0.374 | 2 | ALOX15 |
| MMP10 | 0.705 | 1.542378603 | 0.41 | 0.543 | 0.182 | 2 | MMP10 |
| CD92 | 0.679 | 0.674614935 | 0.358 | 0.752 | 0.582 | 2 | CD9 |
| EGR11 | 0.678 | 0.652734105 | 0.356 | 0.78 | 0.62 | 2 | EGR1 |
| TACSTD22 | 0.673 | 0.738468513 | 0.346 | 0.711 | 0.5 | 2 | TACSTD2 |
| FOS1 | 0.673 | 0.471591371 | 0.346 | 0.948 | 0.883 | 2 | FOS |
| MTRNR2L11 | 0.669 | 0.577117843 | 0.338 | 0.892 | 0.821 | 2 | MTRNR2L1 |
| PERP1 | 0.658 | 0.641025793 | 0.316 | 0.684 | 0.485 | 2 | PERP |
| JUNB | 0.656 | 0.546219638 | 0.312 | 0.811 | 0.738 | 2 | JUNB |
| MIR205HG | 0.655 | 1.170568704 | 0.31 | 0.445 | 0.183 | 2 | MIR205HG |
| LYZ1 | 0.991 | 3.688702213 | 0.982 | 0.999 | 0.296 | 3 | LYZ |
| LTF | 0.986 | 3.254152529 | 0.972 | 0.997 | 0.12 | 3 | LTF |
| ZG16B | 0.979 | 3.731310527 | 0.958 | 0.982 | 0.178 | 3 | ZG16B |
| STATH3 | 0.977 | 3.823603593 | 0.954 | 0.987 | 0.35 | 3 | STATH |
| AZGP1 | 0.974 | 2.636546891 | 0.948 | 0.984 | 0.083 | 3 | AZGP1 |
| BPIFB11 | 0.969 | 2.66682058 | 0.938 | 0.993 | 0.366 | 3 | BPIFB1 |
| SLPI | 0.959 | 2.059771633 | 0.918 | 1 | 0.727 | 3 | SLPI |
| BPIFA11 | 0.958 | 3.304314547 | 0.916 | 0.986 | 0.438 | 3 | BPIFA1 |
| TCN1 | 0.957 | 2.54479442 | 0.914 | 0.956 | 0.094 | 3 | TCN1 |
| PIGR1 | 0.956 | 2.06101259 | 0.912 | 0.995 | 0.299 | 3 | PIGR |
| C6orf58 | 0.944 | 3.584541342 | 0.888 | 0.907 | 0.063 | 3 | C6orf58 |
| DMBT1 | 0.926 | 2.605987453 | 0.852 | 0.887 | 0.057 | 3 | DMBT1 |
| PIP | 0.918 | 3.173329237 | 0.836 | 0.868 | 0.067 | 3 | PIP |
| RP11-1143G9.4 | 0.916 | 3.388481769 | 0.832 | 0.873 | 0.08 | 3 | RP11-1143G9.4 |
| ODAM | 0.903 | 2.460263094 | 0.806 | 0.833 | 0.048 | 3 | ODAM |
| RNASE1 | 0.813 | 1.571996697 | 0.626 | 0.696 | 0.078 | 3 | RNASE1 |
| XBP11 | 0.811 | 1.016598824 | 0.622 | 0.908 | 0.424 | 3 | XBP1 |
| CXCL171 | 0.795 | 1.075006597 | 0.59 | 0.829 | 0.283 | 3 | CXCL17 |
| NUCB2 | 0.779 | 1.071344477 | 0.558 | 0.752 | 0.238 | 3 | NUCB2 |
| CCL28 | 0.764 | 1.340582163 | 0.528 | 0.566 | 0.04 | 3 | CCL28 |
| CA2 | 0.749 | 1.357258619 | 0.498 | 0.526 | 0.028 | 3 | CA2 |
| NDRG2 | 0.742 | 1.047683141 | 0.484 | 0.611 | 0.136 | 3 | NDRG2 |
| PHLDA1 | 0.74 | 1.025974096 | 0.48 | 0.597 | 0.113 | 3 | PHLDA1 |
| SLC12A2 | 0.735 | 1.054028376 | 0.47 | 0.623 | 0.159 | 3 | SLC12A2 |
| WFDC21 | 0.726 | 0.62877281 | 0.452 | 0.857 | 0.425 | 3 | WFDC2 |
| LRRC26 | 0.717 | 1.111806885 | 0.434 | 0.468 | 0.035 | 3 | LRRC26 |
| HP | 0.705 | 1.808087406 | 0.41 | 0.421 | 0.012 | 3 | HP |
| PART1 | 0.705 | 0.975181258 | 0.41 | 0.459 | 0.049 | 3 | PART1 |
| TMED3 | 0.705 | 0.840682234 | 0.41 | 0.567 | 0.162 | 3 | TMED3 |
| PPP1R1B | 0.704 | 1.053103561 | 0.408 | 0.425 | 0.016 | 3 | PPP1R1B |
| AQP5 | 0.701 | 0.776802775 | 0.402 | 0.586 | 0.177 | 3 | AQP5 |
| CLDN10 | 0.695 | 0.900101031 | 0.39 | 0.468 | 0.074 | 3 | CLDN10 |
| FAM3D | 0.695 | 0.828768685 | 0.39 | 0.504 | 0.109 | 3 | FAM3D |
| EHF | 0.695 | 0.66975617 | 0.39 | 0.658 | 0.262 | 3 | EHF |
| S100A1 | 0.691 | 1.022221804 | 0.382 | 0.397 | 0.016 | 3 | S100A1 |
| KIAA1324 | 0.69 | 0.883179039 | 0.38 | 0.467 | 0.087 | 3 | KIAA1324 |
| MGLL | 0.682 | 0.791368708 | 0.364 | 0.499 | 0.132 | 3 | MGLL |
| SCGB3A1 | 0.678 | 1.517195826 | 0.356 | 0.438 | 0.083 | 3 | SCGB3A1 |
| CST3 | 0.673 | 0.301016426 | 0.346 | 0.76 | 0.4 | 3 | CST3 |
| FDCSP | 0.672 | 2.539730671 | 0.344 | 0.387 | 0.052 | 3 | FDCSP |
| P4HB | 0.672 | 0.540962448 | 0.344 | 0.669 | 0.326 | 3 | P4HB |
| LPO | 0.652 | 0.978897961 | 0.304 | 0.309 | 0.006 | 3 | LPO |
| WFDC22 | 0.935 | 1.819752082 | 0.87 | 0.991 | 0.422 | 4 | WFDC2 |
| AGR22 | 0.906 | 1.591362511 | 0.812 | 0.993 | 0.401 | 4 | AGR2 |
| VMO11 | 0.896 | 1.922454988 | 0.792 | 0.92 | 0.246 | 4 | VMO1 |
| SLPI1 | 0.853 | 0.926069168 | 0.706 | 0.999 | 0.731 | 4 | SLPI |
| LYPD2 | 0.849 | 2.331567689 | 0.698 | 0.731 | 0.047 | 4 | LYPD2 |
| PIGR2 | 0.849 | 1.070186025 | 0.698 | 0.975 | 0.311 | 4 | PIGR |
| S100A63 | 0.84 | 0.877882057 | 0.68 | 0.998 | 0.808 | 4 | S100A6 |
| TSPAN11 | 0.835 | 1.277892642 | 0.67 | 0.857 | 0.219 | 4 | TSPAN1 |
| PSCA | 0.828 | 2.199091832 | 0.656 | 0.706 | 0.067 | 4 | PSCA |
| PRSS233 | 0.824 | 1.028620234 | 0.648 | 0.933 | 0.436 | 4 | PRSS23 |
| KRT193 | 0.821 | 0.901493963 | 0.642 | 0.986 | 0.571 | 4 | KRT19 |
| CXCL172 | 0.82 | 1.148837404 | 0.64 | 0.874 | 0.288 | 4 | CXCL17 |
| CP | 0.817 | 1.308804564 | 0.634 | 0.829 | 0.25 | 4 | CP |
| LGALS31 | 0.816 | 0.999347032 | 0.632 | 0.944 | 0.591 | 4 | LGALS3 |
| SCGB1A1 | 0.815 | 2.905152986 | 0.63 | 0.699 | 0.106 | 4 | SCGB1A1 |
| S100P1 | 0.813 | 1.7122795 | 0.626 | 0.75 | 0.163 | 4 | S100P |
| MSMB | 0.812 | 1.988561731 | 0.624 | 0.751 | 0.184 | 4 | MSMB |
| ALDH1A11 | 0.809 | 1.073153499 | 0.618 | 0.875 | 0.329 | 4 | ALDH1A1 |
| ELF32 | 0.806 | 0.99593255 | 0.612 | 0.898 | 0.37 | 4 | ELF3 |
| ALCAM | 0.802 | 1.12564152 | 0.604 | 0.816 | 0.245 | 4 | ALCAM |
| SERPINB33 | 0.795 | 1.055288186 | 0.59 | 0.909 | 0.444 | 4 | SERPINB3 |
| ANXA13 | 0.785 | 0.799082994 | 0.57 | 0.988 | 0.672 | 4 | ANXA1 |
| KRT7 | 0.782 | 1.127567371 | 0.564 | 0.747 | 0.209 | 4 | KRT7 |
| SAT12 | 0.777 | 0.80829761 | 0.554 | 0.951 | 0.627 | 4 | SAT1 |
| BPIFB12 | 0.777 | 0.571742018 | 0.554 | 0.905 | 0.382 | 4 | BPIFB1 |
| UGT2A21 | 0.776 | 1.086387499 | 0.552 | 0.759 | 0.213 | 4 | UGT2A2 |
| S100A4 | 0.775 | 1.188797422 | 0.55 | 0.803 | 0.329 | 4 | S100A4 |
| KRT82 | 0.768 | 0.940825994 | 0.536 | 0.833 | 0.351 | 4 | KRT8 |
| CLDN42 | 0.766 | 0.894690098 | 0.532 | 0.854 | 0.401 | 4 | CLDN4 |
| EPAS11 | 0.766 | 0.89211589 | 0.532 | 0.827 | 0.312 | 4 | EPAS1 |
| MGST1 | 0.758 | 0.910276921 | 0.516 | 0.722 | 0.203 | 4 | MGST1 |
| LCN2 | 0.749 | 1.464821225 | 0.498 | 0.613 | 0.128 | 4 | LCN2 |
| KRT182 | 0.746 | 0.778410807 | 0.492 | 0.813 | 0.352 | 4 | KRT18 |
| CLDN7 | 0.742 | 0.978888754 | 0.484 | 0.634 | 0.154 | 4 | CLDN7 |
| ATP1B1 | 0.74 | 0.835048768 | 0.48 | 0.769 | 0.311 | 4 | ATP1B1 |
| HSPB12 | 0.739 | 0.79759591 | 0.478 | 0.842 | 0.446 | 4 | HSPB1 |
| F31 | 0.738 | 0.751785853 | 0.476 | 0.846 | 0.4 | 4 | F3 |
| PRDX11 | 0.737 | 0.773200739 | 0.474 | 0.86 | 0.5 | 4 | PRDX1 |
| EZR1 | 0.736 | 0.653098759 | 0.472 | 0.897 | 0.502 | 4 | EZR |
| TNFSF10 | 0.733 | 0.893355335 | 0.466 | 0.698 | 0.249 | 4 | TNFSF10 |
| TXN1 | 0.732 | 0.720029058 | 0.464 | 0.848 | 0.48 | 4 | TXN |
| RARRES1 | 0.73 | 1.068402785 | 0.46 | 0.631 | 0.174 | 4 | RARRES1 |
| SPINT2 | 0.73 | 0.701264299 | 0.46 | 0.809 | 0.372 | 4 | SPINT2 |
| CTD-2319I12.11 | 0.728 | 1.208433168 | 0.456 | 0.587 | 0.139 | 4 | CTD-2319I12.1 |
| AQP31 | 0.723 | 0.649680538 | 0.446 | 0.868 | 0.438 | 4 | AQP3 |
| PI3 | 0.721 | 1.369322579 | 0.442 | 0.48 | 0.043 | 4 | PI3 |
| FAM3D1 | 0.721 | 1.004069578 | 0.442 | 0.55 | 0.112 | 4 | FAM3D |
| SLC31A1 | 0.721 | 0.910783722 | 0.442 | 0.59 | 0.154 | 4 | SLC31A1 |
| GABRP1 | 0.72 | 0.86391287 | 0.44 | 0.598 | 0.147 | 4 | GABRP |
| GSTP12 | 0.72 | 0.532572352 | 0.44 | 0.93 | 0.608 | 4 | GSTP1 |
| CD55 | 0.718 | 0.766081725 | 0.436 | 0.782 | 0.383 | 4 | CD55 |
| CTSB1 | 0.718 | 0.567755768 | 0.436 | 0.842 | 0.462 | 4 | CTSB |
| FXYD31 | 0.716 | 0.570100391 | 0.432 | 0.905 | 0.502 | 4 | FXYD3 |
| CYP4B1 | 0.715 | 0.928394643 | 0.43 | 0.575 | 0.136 | 4 | CYP4B1 |
| POR | 0.715 | 0.881470345 | 0.43 | 0.565 | 0.131 | 4 | POR |
| CYB5A | 0.714 | 0.826253088 | 0.428 | 0.611 | 0.185 | 4 | CYB5A |
| TMEM213 | 0.712 | 1.224166658 | 0.424 | 0.487 | 0.063 | 4 | TMEM213 |
| STEAP4 | 0.711 | 1.171567545 | 0.422 | 0.473 | 0.052 | 4 | STEAP4 |
| TACSTD23 | 0.711 | 0.537305357 | 0.422 | 0.881 | 0.492 | 4 | TACSTD2 |
| GDF15 | 0.706 | 1.343960146 | 0.412 | 0.49 | 0.083 | 4 | GDF15 |
| CLINT1 | 0.704 | 0.690890334 | 0.408 | 0.661 | 0.259 | 4 | CLINT1 |
| S100A14 | 0.703 | 0.946914912 | 0.406 | 0.496 | 0.09 | 4 | S100A14 |
| PLAC8 | 0.703 | 0.83437864 | 0.406 | 0.517 | 0.101 | 4 | PLAC8 |
| MSLN | 0.702 | 1.107704786 | 0.404 | 0.482 | 0.08 | 4 | MSLN |
| IER3 | 0.701 | 0.648278432 | 0.402 | 0.776 | 0.395 | 4 | IER3 |
| IFI27 | 0.698 | 0.79098078 | 0.396 | 0.65 | 0.264 | 4 | IFI27 |
| MDK | 0.698 | 0.692523077 | 0.396 | 0.639 | 0.247 | 4 | MDK |
| CSTB1 | 0.697 | 0.650007534 | 0.394 | 0.698 | 0.305 | 4 | CSTB |
| SORD | 0.695 | 0.910134577 | 0.39 | 0.482 | 0.092 | 4 | SORD |
| DUSP12 | 0.693 | 0.50215056 | 0.386 | 0.923 | 0.714 | 4 | DUSP1 |
| NTS | 0.69 | 1.201855393 | 0.38 | 0.545 | 0.182 | 4 | NTS |
| ALDH3A11 | 0.69 | 0.8855255 | 0.38 | 0.609 | 0.235 | 4 | ALDH3A1 |
| AQP51 | 0.69 | 0.799787852 | 0.38 | 0.565 | 0.184 | 4 | AQP5 |
| S100A11 | 0.69 | 0.51000193 | 0.38 | 0.855 | 0.535 | 4 | S100A11 |
| ANXA22 | 0.689 | 0.468936375 | 0.378 | 0.896 | 0.571 | 4 | ANXA2 |
| LY6E | 0.688 | 0.667464923 | 0.376 | 0.58 | 0.2 | 4 | LY6E |
| TOB1 | 0.688 | 0.59736805 | 0.376 | 0.698 | 0.315 | 4 | TOB1 |
| MUC16 | 0.686 | 1.060288488 | 0.372 | 0.421 | 0.049 | 4 | MUC16 |
| HES11 | 0.685 | 0.640229047 | 0.37 | 0.699 | 0.345 | 4 | HES1 |
| ST6GALNAC1 | 0.683 | 0.728652442 | 0.366 | 0.494 | 0.119 | 4 | ST6GALNAC1 |
| SLC9A3R1 | 0.683 | 0.635539885 | 0.366 | 0.555 | 0.176 | 4 | SLC9A3R1 |
| PERP2 | 0.683 | 0.44916993 | 0.366 | 0.857 | 0.477 | 4 | PERP |
| CTSD | 0.682 | 0.547147224 | 0.364 | 0.694 | 0.328 | 4 | CTSD |
| KLF51 | 0.681 | 0.565560482 | 0.362 | 0.642 | 0.258 | 4 | KLF5 |
| CST31 | 0.681 | 0.366818459 | 0.362 | 0.784 | 0.404 | 4 | CST3 |
| C8orf4 | 0.68 | 0.853971251 | 0.36 | 0.523 | 0.164 | 4 | C8orf4 |
| RIMS1 | 0.678 | 1.04389864 | 0.356 | 0.415 | 0.06 | 4 | RIMS1 |
| ADAM28 | 0.678 | 0.634744633 | 0.356 | 0.576 | 0.209 | 4 | ADAM28 |
| RDH10 | 0.676 | 0.817352829 | 0.352 | 0.465 | 0.11 | 4 | RDH10 |
| S100A13 | 0.676 | 0.632842954 | 0.352 | 0.562 | 0.202 | 4 | S100A13 |
| MUC1 | 0.675 | 0.924941423 | 0.35 | 0.426 | 0.077 | 4 | MUC1 |
| ASS1 | 0.675 | 0.834110944 | 0.35 | 0.462 | 0.113 | 4 | ASS1 |
| ASAH1 | 0.675 | 0.564179979 | 0.35 | 0.595 | 0.234 | 4 | ASAH1 |
| EPHX1 | 0.673 | 0.66023497 | 0.346 | 0.506 | 0.154 | 4 | EPHX1 |
| OAT | 0.673 | 0.592019535 | 0.346 | 0.601 | 0.248 | 4 | OAT |
| ACTG12 | 0.672 | 0.394196892 | 0.344 | 0.918 | 0.673 | 4 | ACTG1 |
| MT1E | 0.671 | 0.64690952 | 0.342 | 0.579 | 0.222 | 4 | MT1E |
| CD82 | 0.67 | 0.642973291 | 0.34 | 0.489 | 0.144 | 4 | CD82 |
| TSPAN8 | 0.669 | 1.070393632 | 0.338 | 0.375 | 0.038 | 4 | TSPAN8 |
| SELENBP1 | 0.668 | 0.728707579 | 0.336 | 0.451 | 0.113 | 4 | SELENBP1 |
| C19orf33 | 0.668 | 0.613641075 | 0.336 | 0.529 | 0.177 | 4 | C19orf33 |
| VAMP8 | 0.668 | 0.497636227 | 0.336 | 0.695 | 0.356 | 4 | VAMP8 |
| PPDPF | 0.668 | 0.48689716 | 0.336 | 0.655 | 0.307 | 4 | PPDPF |
| DHCR24 | 0.666 | 0.598215437 | 0.332 | 0.51 | 0.165 | 4 | DHCR24 |
| ATF31 | 0.666 | 0.496899243 | 0.332 | 0.774 | 0.474 | 4 | ATF3 |
| FMO3 | 0.665 | 0.794413575 | 0.33 | 0.475 | 0.137 | 4 | FMO3 |
| DHRS9 | 0.664 | 0.878570137 | 0.328 | 0.376 | 0.045 | 4 | DHRS9 |
| CYP2A13 | 0.663 | 1.173927627 | 0.326 | 0.345 | 0.018 | 4 | CYP2A13 |
| XBP12 | 0.662 | 0.438522373 | 0.324 | 0.802 | 0.439 | 4 | XBP1 |
| APLP2 | 0.662 | 0.422306914 | 0.324 | 0.724 | 0.373 | 4 | APLP2 |
| SLC6A141 | 0.659 | 0.723575453 | 0.318 | 0.453 | 0.128 | 4 | SLC6A14 |
| MAFF | 0.659 | 0.575390336 | 0.318 | 0.545 | 0.218 | 4 | MAFF |
| SLC5A8 | 0.658 | 0.690881865 | 0.316 | 0.442 | 0.123 | 4 | SLC5A8 |
| GSN1 | 0.658 | 0.517488093 | 0.316 | 0.58 | 0.236 | 4 | GSN |
| LAMTOR5 | 0.657 | 0.483299753 | 0.314 | 0.62 | 0.291 | 4 | LAMTOR5 |
| CAPN13 | 0.656 | 0.673440766 | 0.312 | 0.429 | 0.109 | 4 | CAPN13 |
| RTN3 | 0.656 | 0.522442371 | 0.312 | 0.529 | 0.211 | 4 | RTN3 |
| SDC4 | 0.653 | 0.537544969 | 0.306 | 0.51 | 0.194 | 4 | SDC4 |
| TSPAN3 | 0.653 | 0.474999053 | 0.306 | 0.575 | 0.252 | 4 | TSPAN3 |
| CTSC | 0.653 | 0.36255651 | 0.306 | 0.614 | 0.27 | 4 | CTSC |
| CYP2F1 | 0.652 | 1.129272589 | 0.304 | 0.318 | 0.016 | 4 | CYP2F1 |
| SCNN1A | 0.652 | 0.62679136 | 0.304 | 0.41 | 0.1 | 4 | SCNN1A |
| RAB11FIP1 | 0.652 | 0.600039684 | 0.304 | 0.464 | 0.149 | 4 | RAB11FIP1 |
| TMPRSS4 | 0.652 | 0.598964298 | 0.304 | 0.417 | 0.104 | 4 | TMPRSS4 |
| RHOB | 0.651 | 0.486452693 | 0.302 | 0.607 | 0.291 | 4 | RHOB |
| DCN | 0.913 | 2.607755142 | 0.826 | 0.883 | 0.109 | 5 | DCN |
| COL1A2 | 0.843 | 2.193941789 | 0.686 | 0.74 | 0.072 | 5 | COL1A2 |
| LUM | 0.836 | 2.303783413 | 0.672 | 0.733 | 0.094 | 5 | LUM |
| COL3A1 | 0.83 | 2.373717147 | 0.66 | 0.713 | 0.075 | 5 | COL3A1 |
| MGP | 0.824 | 2.29465976 | 0.648 | 0.72 | 0.12 | 5 | MGP |
| LGALS1 | 0.819 | 1.759115609 | 0.638 | 0.76 | 0.205 | 5 | LGALS1 |
| IGFBP7 | 0.799 | 1.669459622 | 0.598 | 0.752 | 0.27 | 5 | IGFBP7 |
| CALD1 | 0.788 | 1.864239284 | 0.576 | 0.653 | 0.112 | 5 | CALD1 |
| CPE | 0.778 | 1.985323658 | 0.556 | 0.609 | 0.074 | 5 | CPE |
| FBLN1 | 0.771 | 1.939605437 | 0.542 | 0.601 | 0.084 | 5 | FBLN1 |
| VIM2 | 0.77 | 1.160895469 | 0.54 | 0.78 | 0.326 | 5 | VIM |
| SPARC | 0.767 | 1.802375177 | 0.534 | 0.621 | 0.113 | 5 | SPARC |
| POSTN4 | 0.74 | 1.219992749 | 0.48 | 0.777 | 0.43 | 5 | POSTN |
| SFRP2 | 0.727 | 2.019824578 | 0.454 | 0.491 | 0.047 | 5 | SFRP2 |
| IFITM3 | 0.727 | 1.16780664 | 0.454 | 0.683 | 0.344 | 5 | IFITM3 |
| C1S | 0.71 | 1.726090513 | 0.42 | 0.467 | 0.058 | 5 | C1S |
| SFRP1 | 0.703 | 1.728998219 | 0.406 | 0.466 | 0.075 | 5 | SFRP1 |
| SERPING1 | 0.7 | 1.634965654 | 0.4 | 0.456 | 0.073 | 5 | SERPING1 |
| SEPP1 | 0.697 | 1.258262751 | 0.394 | 0.584 | 0.292 | 5 | SEPP1 |
| COL1A1 | 0.696 | 1.764752104 | 0.392 | 0.434 | 0.05 | 5 | COL1A1 |
| TAGLN | 0.695 | 1.871149653 | 0.39 | 0.442 | 0.064 | 5 | TAGLN |
| AEBP1 | 0.688 | 1.669164092 | 0.376 | 0.42 | 0.053 | 5 | AEBP1 |
| PCOLCE | 0.688 | 1.625272847 | 0.376 | 0.414 | 0.046 | 5 | PCOLCE |
| C1R | 0.682 | 1.533306023 | 0.364 | 0.419 | 0.069 | 5 | C1R |
| PPAP2B | 0.681 | 1.490882731 | 0.362 | 0.45 | 0.121 | 5 | PPAP2B |
| TPM2 | 0.669 | 1.615857208 | 0.338 | 0.393 | 0.067 | 5 | TPM2 |
| IGFBP6 | 0.668 | 1.711513409 | 0.336 | 0.374 | 0.048 | 5 | IGFBP6 |
| TMSB4X2 | 0.666 | 0.46770672 | 0.332 | 0.833 | 0.686 | 5 | TMSB4X |
| CRABP2 | 0.665 | 1.585476354 | 0.33 | 0.367 | 0.042 | 5 | CRABP2 |
| TPM1 | 0.662 | 1.492013957 | 0.324 | 0.421 | 0.132 | 5 | TPM1 |
| CXCL14 | 0.661 | 1.916311865 | 0.322 | 0.36 | 0.045 | 5 | CXCL14 |
| SELM | 0.657 | 1.114732133 | 0.314 | 0.513 | 0.292 | 5 | SELM |
| CDH11 | 0.656 | 1.53193819 | 0.312 | 0.351 | 0.048 | 5 | CDH11 |
| CLDN11 | 0.654 | 1.569586829 | 0.308 | 0.338 | 0.036 | 5 | CLDN11 |
| THY1 | 0.654 | 1.525223482 | 0.308 | 0.341 | 0.038 | 5 | THY1 |
| PRRX1 | 0.653 | 1.489554638 | 0.306 | 0.337 | 0.038 | 5 | PRRX1 |
| RARRES2 | 0.653 | 1.46921555 | 0.306 | 0.349 | 0.051 | 5 | RARRES2 |
| S100A64 | 0.653 | 0.401241006 | 0.306 | 0.928 | 0.813 | 5 | S100A6 |
| SPARCL1 | 0.927 | 2.628272794 | 0.854 | 0.89 | 0.13 | 6 | SPARCL1 |
| VIM3 | 0.88 | 1.606546153 | 0.76 | 0.917 | 0.318 | 6 | VIM |
| HLA-E | 0.869 | 1.331402761 | 0.738 | 0.925 | 0.591 | 6 | HLA-E |
| GNG11 | 0.86 | 2.242840748 | 0.72 | 0.765 | 0.078 | 6 | GNG11 |
| A2M | 0.839 | 2.142195955 | 0.678 | 0.721 | 0.065 | 6 | A2M |
| TMSB101 | 0.837 | 0.954369076 | 0.674 | 0.969 | 0.81 | 6 | TMSB10 |
| IFI271 | 0.825 | 1.584527993 | 0.65 | 0.798 | 0.256 | 6 | IFI27 |
| IGFBP71 | 0.818 | 1.373198956 | 0.636 | 0.815 | 0.267 | 6 | IGFBP7 |
| IFITM31 | 0.812 | 1.290604411 | 0.624 | 0.827 | 0.335 | 6 | IFITM3 |
| CD742 | 0.805 | 0.906898611 | 0.61 | 0.859 | 0.431 | 6 | CD74 |
| CLDN5 | 0.793 | 2.426446416 | 0.586 | 0.596 | 0.015 | 6 | CLDN5 |
| ELTD1 | 0.789 | 2.01511919 | 0.578 | 0.587 | 0.011 | 6 | ELTD1 |
| TMSB4X3 | 0.771 | 0.683083677 | 0.542 | 0.946 | 0.679 | 6 | TMSB4X |
| DARC | 0.768 | 2.38801981 | 0.536 | 0.548 | 0.019 | 6 | DARC |
| EMCN | 0.768 | 2.004852333 | 0.536 | 0.542 | 0.008 | 6 | EMCN |
| TM4SF1 | 0.76 | 1.460798846 | 0.52 | 0.662 | 0.205 | 6 | TM4SF1 |
| SPARC1 | 0.754 | 1.31673386 | 0.508 | 0.619 | 0.113 | 6 | SPARC |
| PTRF | 0.745 | 1.36313013 | 0.49 | 0.591 | 0.116 | 6 | PTRF |
| VWF | 0.731 | 1.876940364 | 0.462 | 0.474 | 0.015 | 6 | VWF |
| GIMAP7 | 0.726 | 1.607288978 | 0.452 | 0.477 | 0.026 | 6 | GIMAP7 |
| IFITM2 | 0.725 | 1.121570797 | 0.45 | 0.606 | 0.188 | 6 | IFITM2 |
| PLVAP | 0.722 | 1.773762368 | 0.444 | 0.449 | 0.006 | 6 | PLVAP |
| RPL33 | 0.72 | 0.570513097 | 0.44 | 0.936 | 0.834 | 6 | RPL3 |
| RPS23 | 0.72 | 0.555187604 | 0.44 | 0.927 | 0.829 | 6 | RPS23 |
| ECSCR | 0.719 | 1.609592981 | 0.438 | 0.444 | 0.006 | 6 | ECSCR |
| EMP1 | 0.717 | 1.316439981 | 0.434 | 0.56 | 0.153 | 6 | EMP1 |
| RPL32 | 0.716 | 0.513959559 | 0.432 | 0.941 | 0.865 | 6 | RPL32 |
| RAMP2 | 0.714 | 1.578512844 | 0.428 | 0.437 | 0.01 | 6 | RAMP2 |
| CALCRL | 0.714 | 1.544245814 | 0.428 | 0.457 | 0.032 | 6 | CALCRL |
| HLA-DRB1 | 0.713 | 0.628056508 | 0.426 | 0.594 | 0.184 | 6 | HLA-DRB1 |
| PTMA1 | 0.709 | 0.594751336 | 0.418 | 0.88 | 0.689 | 6 | PTMA |
| ID3 | 0.706 | 1.051042388 | 0.412 | 0.615 | 0.263 | 6 | ID3 |
| ESAM | 0.705 | 1.541258446 | 0.41 | 0.418 | 0.009 | 6 | ESAM |
| RPL31 | 0.705 | 0.432534266 | 0.41 | 0.973 | 0.921 | 6 | RPL31 |
| HLA-DRA | 0.705 | 0.339578135 | 0.41 | 0.621 | 0.243 | 6 | HLA-DRA |
| APOLD1 | 0.703 | 1.734903113 | 0.406 | 0.416 | 0.012 | 6 | APOLD1 |
| ADAMTS1 | 0.698 | 1.583270956 | 0.396 | 0.442 | 0.05 | 6 | ADAMTS1 |
| FKBP1A | 0.698 | 1.264096468 | 0.396 | 0.498 | 0.132 | 6 | FKBP1A |
| ADIRF | 0.693 | 1.071548064 | 0.386 | 0.623 | 0.329 | 6 | ADIRF |
| RAMP3 | 0.691 | 1.559134677 | 0.382 | 0.388 | 0.008 | 6 | RAMP3 |
| RPS27A | 0.689 | 0.390946892 | 0.378 | 0.958 | 0.899 | 6 | RPS27A |
| EGFL7 | 0.688 | 1.484811567 | 0.376 | 0.393 | 0.02 | 6 | EGFL7 |
| RPS15A1 | 0.688 | 0.422753885 | 0.376 | 0.949 | 0.853 | 6 | RPS15A |
| HLA-B | 0.687 | 0.455635516 | 0.374 | 0.919 | 0.817 | 6 | HLA-B |
| IL33 | 0.685 | 1.028094361 | 0.37 | 0.549 | 0.212 | 6 | IL33 |
| RPS13 | 0.684 | 0.392891849 | 0.368 | 0.954 | 0.886 | 6 | RPS13 |
| RPS3 | 0.683 | 0.440142311 | 0.366 | 0.907 | 0.804 | 6 | RPS3 |
| LIFR | 0.682 | 1.408312795 | 0.364 | 0.395 | 0.039 | 6 | LIFR |
| CAV1 | 0.682 | 1.321945786 | 0.364 | 0.421 | 0.064 | 6 | CAV1 |
| NPDC1 | 0.682 | 1.307439011 | 0.364 | 0.42 | 0.07 | 6 | NPDC1 |
| CD34 | 0.681 | 1.393630883 | 0.362 | 0.375 | 0.015 | 6 | CD34 |
| AC011526.1 | 0.68 | 1.400954225 | 0.36 | 0.366 | 0.006 | 6 | AC011526.1 |
| NOSTRIN | 0.675 | 1.437197586 | 0.35 | 0.36 | 0.012 | 6 | NOSTRIN |
| IL6ST | 0.675 | 0.855877879 | 0.35 | 0.575 | 0.269 | 6 | IL6ST |
| RPL24 | 0.675 | 0.465106499 | 0.35 | 0.867 | 0.739 | 6 | RPL24 |
| RPS91 | 0.675 | 0.449328342 | 0.35 | 0.894 | 0.794 | 6 | RPS9 |
| CYYR1 | 0.672 | 1.252004493 | 0.344 | 0.363 | 0.021 | 6 | CYYR1 |
| CRIP2 | 0.671 | 1.231644639 | 0.342 | 0.392 | 0.062 | 6 | CRIP2 |
| RDX | 0.671 | 1.132695519 | 0.342 | 0.464 | 0.149 | 6 | RDX |
| JAM2 | 0.67 | 1.314552633 | 0.34 | 0.353 | 0.013 | 6 | JAM2 |
| RPL5 | 0.669 | 0.454097444 | 0.338 | 0.885 | 0.782 | 6 | RPL5 |
| TGFBR2 | 0.668 | 1.203488109 | 0.336 | 0.389 | 0.059 | 6 | TGFBR2 |
| STOM | 0.666 | 0.989328277 | 0.332 | 0.454 | 0.136 | 6 | STOM |
| TPM3 | 0.665 | 0.762352188 | 0.33 | 0.566 | 0.278 | 6 | TPM3 |
| TXNIP | 0.663 | 0.636346865 | 0.326 | 0.728 | 0.511 | 6 | TXNIP |
| TSPAN7 | 0.662 | 1.385194017 | 0.324 | 0.331 | 0.007 | 6 | TSPAN7 |
| ENG | 0.662 | 1.252859784 | 0.324 | 0.346 | 0.024 | 6 | ENG |
| HLA-DPA1 | 0.662 | 0.46895486 | 0.324 | 0.451 | 0.126 | 6 | HLA-DPA1 |
| RPL26 | 0.661 | 0.425596102 | 0.322 | 0.878 | 0.783 | 6 | RPL26 |
| SPRY1 | 0.66 | 1.244412172 | 0.32 | 0.393 | 0.084 | 6 | SPRY1 |
| SPTBN1 | 0.657 | 0.99322469 | 0.314 | 0.423 | 0.126 | 6 | SPTBN1 |
| EEF1A1 | 0.657 | 0.544498056 | 0.314 | 0.773 | 0.623 | 6 | EEF1A1 |
| PALMD | 0.656 | 1.229873161 | 0.312 | 0.366 | 0.062 | 6 | PALMD |
| RPL9 | 0.656 | 0.580986638 | 0.312 | 0.74 | 0.56 | 6 | RPL9 |
| SOCS3 | 0.655 | 0.706973765 | 0.31 | 0.58 | 0.313 | 6 | SOCS3 |
| CD93 | 0.654 | 1.205490803 | 0.308 | 0.322 | 0.013 | 6 | CD93 |
| ELK3 | 0.654 | 1.097854213 | 0.308 | 0.358 | 0.054 | 6 | ELK3 |
| SELE | 0.652 | 2.012295023 | 0.304 | 0.31 | 0.007 | 6 | SELE |
| KCTD12 | 0.652 | 1.150915158 | 0.304 | 0.343 | 0.041 | 6 | KCTD12 |
| MYL12A | 0.652 | 0.662754662 | 0.304 | 0.64 | 0.413 | 6 | MYL12A |
| IGJ3 | 0.938 | 2.540069034 | 0.876 | 0.977 | 0.464 | 7 | IGJ |
| SSR41 | 0.927 | 1.65457134 | 0.854 | 0.982 | 0.554 | 7 | SSR4 |
| IGKC | 0.912 | 4.0604376 | 0.824 | 0.886 | 0.187 | 7 | IGKC |
| MZB1 | 0.9 | 1.975237891 | 0.8 | 0.897 | 0.127 | 7 | MZB1 |
| IGHA13 | 0.882 | 2.738783811 | 0.764 | 0.951 | 0.537 | 7 | IGHA1 |
| SEC11C | 0.856 | 1.503925044 | 0.712 | 0.871 | 0.302 | 7 | SEC11C |
| ENAM | 0.85 | 2.386366364 | 0.7 | 0.766 | 0.088 | 7 | ENAM |
| HSP90B1 | 0.838 | 1.231683391 | 0.676 | 0.927 | 0.567 | 7 | HSP90B1 |
| IGHA2 | 0.827 | 2.830342514 | 0.654 | 0.773 | 0.215 | 7 | IGHA2 |
| HERPUD1 | 0.817 | 1.428242941 | 0.634 | 0.819 | 0.313 | 7 | HERPUD1 |
| AC096579.7 | 0.815 | 2.808130812 | 0.63 | 0.687 | 0.064 | 7 | AC096579.7 |
| DERL3 | 0.796 | 1.68896373 | 0.592 | 0.663 | 0.081 | 7 | DERL3 |
| SPCS3 | 0.787 | 1.171510384 | 0.574 | 0.79 | 0.331 | 7 | SPCS3 |
| RGS1 | 0.779 | 1.449047836 | 0.558 | 0.708 | 0.171 | 7 | RGS1 |
| PRDX4 | 0.778 | 1.252987028 | 0.556 | 0.737 | 0.261 | 7 | PRDX4 |
| RNA28S5 | 0.772 | 0.748854191 | 0.544 | 0.944 | 0.784 | 7 | RNA28S5 |
| FKBP11 | 0.763 | 1.394426829 | 0.526 | 0.625 | 0.118 | 7 | FKBP11 |
| TSC22D3 | 0.758 | 1.094025659 | 0.516 | 0.781 | 0.387 | 7 | TSC22D3 |
| SLAMF7 | 0.75 | 1.450365916 | 0.5 | 0.556 | 0.058 | 7 | SLAMF7 |
| SSR3 | 0.749 | 1.115710646 | 0.498 | 0.706 | 0.287 | 7 | SSR3 |
| FAM46C | 0.736 | 1.248850266 | 0.472 | 0.584 | 0.133 | 7 | FAM46C |
| XBP15 | 0.727 | 0.846139287 | 0.454 | 0.803 | 0.441 | 7 | XBP1 |
| PIM2 | 0.716 | 1.351203816 | 0.432 | 0.504 | 0.077 | 7 | PIM2 |
| IGHG3 | 0.713 | 2.187278048 | 0.426 | 0.59 | 0.207 | 7 | IGHG3 |
| IGHG1 | 0.709 | 1.817542967 | 0.418 | 0.577 | 0.192 | 7 | IGHG1 |
| UBE2J1 | 0.704 | 1.155032498 | 0.408 | 0.531 | 0.146 | 7 | UBE2J1 |
| CD79A | 0.703 | 1.28666287 | 0.406 | 0.456 | 0.051 | 7 | CD79A |
| SEL1L | 0.7 | 1.136654816 | 0.4 | 0.525 | 0.149 | 7 | SEL1L |
| TRAM1 | 0.699 | 0.81732587 | 0.398 | 0.665 | 0.333 | 7 | TRAM1 |
| RRBP1 | 0.696 | 0.959684948 | 0.392 | 0.623 | 0.296 | 7 | RRBP1 |
| ITM2C | 0.693 | 1.10601361 | 0.386 | 0.511 | 0.147 | 7 | ITM2C |
| SRGN1 | 0.692 | 0.304864587 | 0.384 | 0.709 | 0.298 | 7 | SRGN |
| SUB1 | 0.691 | 0.8289714 | 0.382 | 0.648 | 0.324 | 7 | SUB1 |
| SPCS1 | 0.689 | 0.826976582 | 0.378 | 0.641 | 0.336 | 7 | SPCS1 |
| IGKV1OR2-108 | 0.687 | 2.541423639 | 0.374 | 0.378 | 0.004 | 7 | IGKV1OR2-108 |
| IGKV4-1 | 0.682 | 3.631360774 | 0.364 | 0.398 | 0.037 | 7 | IGKV4-1 |
| SELK | 0.68 | 0.771143723 | 0.36 | 0.639 | 0.338 | 7 | SELK |
| ERLEC1 | 0.678 | 0.831134153 | 0.356 | 0.563 | 0.242 | 7 | ERLEC1 |
| TNFRSF17 | 0.677 | 1.292880358 | 0.354 | 0.385 | 0.032 | 7 | TNFRSF17 |
| FCRL5 | 0.677 | 1.150988511 | 0.354 | 0.396 | 0.042 | 7 | FCRL5 |
| VIMP | 0.676 | 0.835960236 | 0.352 | 0.579 | 0.27 | 7 | VIMP |
| PRDM1 | 0.675 | 1.162724 | 0.35 | 0.42 | 0.072 | 7 | PRDM1 |
| ISG20 | 0.675 | 1.055067052 | 0.35 | 0.456 | 0.116 | 7 | ISG20 |
| CYBA | 0.675 | 0.906978274 | 0.35 | 0.511 | 0.181 | 7 | CYBA |
| IGLV3-1 | 0.674 | 0.568864075 | 0.348 | 0.383 | 0.036 | 7 | IGLV3-1 |
| FKBP2 | 0.669 | 0.830611682 | 0.338 | 0.535 | 0.228 | 7 | FKBP2 |
| DNAJB9 | 0.668 | 0.999032674 | 0.336 | 0.45 | 0.126 | 7 | DNAJB9 |
| IGHG4 | 0.667 | 1.481784528 | 0.334 | 0.529 | 0.229 | 7 | IGHG4 |
| SPCS2 | 0.667 | 0.827519506 | 0.334 | 0.493 | 0.175 | 7 | SPCS2 |
| IGKV1-5 | 0.664 | 3.542564711 | 0.328 | 0.341 | 0.013 | 7 | IGKV1-5 |
| IGHG2 | 0.66 | 2.124053434 | 0.32 | 0.416 | 0.112 | 7 | IGHG2 |
| SEL1L3 | 0.658 | 1.006476852 | 0.316 | 0.385 | 0.073 | 7 | SEL1L3 |
| SEC61B | 0.657 | 0.685384238 | 0.314 | 0.61 | 0.348 | 7 | SEC61B |
| PDIA6 | 0.656 | 0.633475072 | 0.312 | 0.639 | 0.381 | 7 | PDIA6 |
| PDIA4 | 0.654 | 0.872916347 | 0.308 | 0.476 | 0.194 | 7 | PDIA4 |
| SDF2L1 | 0.653 | 0.953689407 | 0.306 | 0.393 | 0.095 | 7 | SDF2L1 |
| C19orf10 | 0.653 | 0.763775855 | 0.306 | 0.521 | 0.246 | 7 | C19orf10 |
| PPAPDC1B | 0.652 | 0.873932763 | 0.304 | 0.432 | 0.141 | 7 | PPAPDC1B |
| PPIB | 0.652 | 0.676156996 | 0.304 | 0.622 | 0.382 | 7 | PPIB |
| CRELD2 | 0.651 | 0.908012241 | 0.302 | 0.403 | 0.112 | 7 | CRELD2 |
| KRT51 | 0.855 | 1.299950148 | 0.71 | 0.912 | 0.234 | 8 | KRT5 |
| AQP35 | 0.823 | 1.044563848 | 0.646 | 0.946 | 0.441 | 8 | AQP3 |
| TSC22D11 | 0.801 | 1.138596109 | 0.602 | 0.868 | 0.358 | 8 | TSC22D1 |
| ALDH3A12 | 0.79 | 1.134316345 | 0.58 | 0.793 | 0.231 | 8 | ALDH3A1 |
| KRT151 | 0.788 | 1.263156118 | 0.576 | 0.761 | 0.205 | 8 | KRT15 |
| S100A22 | 0.786 | 0.701344021 | 0.572 | 0.921 | 0.355 | 8 | S100A2 |
| F34 | 0.78 | 0.903512794 | 0.56 | 0.903 | 0.404 | 8 | F3 |
| FOS3 | 0.776 | 0.585662757 | 0.552 | 0.998 | 0.882 | 8 | FOS |
| PERP6 | 0.772 | 0.765805706 | 0.544 | 0.93 | 0.479 | 8 | PERP |
| KRT197 | 0.769 | 0.720592566 | 0.538 | 0.972 | 0.578 | 8 | KRT19 |
| EGR14 | 0.767 | 0.737562611 | 0.534 | 0.961 | 0.616 | 8 | EGR1 |
| EPAS13 | 0.758 | 0.847232884 | 0.516 | 0.811 | 0.321 | 8 | EPAS1 |
| GLUL2 | 0.758 | 0.761970811 | 0.516 | 0.883 | 0.441 | 8 | GLUL |
| DST | 0.755 | 1.065282225 | 0.51 | 0.702 | 0.199 | 8 | DST |
| ID12 | 0.749 | 0.763982634 | 0.498 | 0.873 | 0.446 | 8 | ID1 |
| MT1X2 | 0.748 | 0.779693354 | 0.496 | 0.786 | 0.299 | 8 | MT1X |
| ALDH3A2 | 0.741 | 0.897288774 | 0.482 | 0.693 | 0.212 | 8 | ALDH3A2 |
| MIR205HG1 | 0.738 | 0.886740862 | 0.476 | 0.667 | 0.18 | 8 | MIR205HG |
| SPINK5 | 0.729 | 1.575736236 | 0.458 | 0.51 | 0.055 | 8 | SPINK5 |
| PRSS235 | 0.726 | 0.596491222 | 0.452 | 0.889 | 0.446 | 8 | PRSS23 |
| ZFP36L12 | 0.721 | 0.611691346 | 0.442 | 0.884 | 0.5 | 8 | ZFP36L1 |
| CLDN1 | 0.718 | 0.776323285 | 0.436 | 0.627 | 0.176 | 8 | CLDN1 |
| TP63 | 0.717 | 0.973746902 | 0.434 | 0.537 | 0.092 | 8 | TP63 |
| JUNB3 | 0.715 | 0.520674306 | 0.43 | 0.962 | 0.732 | 8 | JUNB |
| RPL301 | 0.715 | 0.436102153 | 0.43 | 0.987 | 0.909 | 8 | RPL30 |
| KRT17 | 0.714 | 0.807942984 | 0.428 | 0.589 | 0.141 | 8 | KRT17 |
| RPS4X | 0.712 | 0.448745109 | 0.424 | 0.974 | 0.857 | 8 | RPS4X |
| PABPC12 | 0.711 | 0.446058271 | 0.422 | 0.97 | 0.773 | 8 | PABPC1 |
| EGFR | 0.708 | 0.850104125 | 0.416 | 0.543 | 0.121 | 8 | EGFR |
| JUN | 0.705 | 0.453787151 | 0.41 | 0.976 | 0.763 | 8 | JUN |
| C17orf76-AS11 | 0.703 | 0.586349941 | 0.406 | 0.832 | 0.494 | 8 | C17orf76-AS1 |
| IL331 | 0.698 | 0.648012887 | 0.396 | 0.629 | 0.212 | 8 | IL33 |
| ATF32 | 0.697 | 0.632882293 | 0.394 | 0.82 | 0.476 | 8 | ATF3 |
| IER32 | 0.697 | 0.568332414 | 0.394 | 0.785 | 0.4 | 8 | IER3 |
| DSP | 0.696 | 0.665093009 | 0.392 | 0.619 | 0.216 | 8 | DSP |
| KLF52 | 0.695 | 0.630665623 | 0.39 | 0.668 | 0.263 | 8 | KLF5 |
| RASSF6 | 0.691 | 0.806159744 | 0.382 | 0.506 | 0.116 | 8 | RASSF6 |
| CYR61 | 0.69 | 0.744617994 | 0.38 | 0.585 | 0.203 | 8 | CYR61 |
| ETS2 | 0.69 | 0.524202607 | 0.38 | 0.746 | 0.344 | 8 | ETS2 |
| ADH7 | 0.688 | 0.78878588 | 0.376 | 0.515 | 0.133 | 8 | ADH7 |
| PRNP | 0.687 | 0.672118169 | 0.374 | 0.574 | 0.184 | 8 | PRNP |
| RPS31 | 0.685 | 0.377962744 | 0.37 | 0.951 | 0.803 | 8 | RPS3 |
| FOSB | 0.683 | 0.472253222 | 0.366 | 0.904 | 0.617 | 8 | FOSB |
| MPZL2 | 0.681 | 0.566771586 | 0.362 | 0.566 | 0.184 | 8 | MPZL2 |
| DNAJB1 | 0.681 | 0.556020032 | 0.362 | 0.695 | 0.339 | 8 | DNAJB1 |
| ZFAS11 | 0.681 | 0.478206698 | 0.362 | 0.811 | 0.474 | 8 | ZFAS1 |
| HSP90AB1 | 0.681 | 0.437138149 | 0.362 | 0.876 | 0.556 | 8 | HSP90AB1 |
| UGT2A22 | 0.68 | 0.615969385 | 0.36 | 0.607 | 0.229 | 8 | UGT2A2 |
| SLC38A2 | 0.68 | 0.586613532 | 0.36 | 0.695 | 0.336 | 8 | SLC38A2 |
| TXNIP1 | 0.68 | 0.457227698 | 0.36 | 0.84 | 0.508 | 8 | TXNIP |
| GAS5 | 0.677 | 0.483843612 | 0.354 | 0.82 | 0.497 | 8 | GAS5 |
| HES13 | 0.676 | 0.556547626 | 0.352 | 0.71 | 0.35 | 8 | HES1 |
| GNB2L1 | 0.675 | 0.372945317 | 0.35 | 0.938 | 0.747 | 8 | GNB2L1 |
| BCAM | 0.673 | 0.769533026 | 0.346 | 0.452 | 0.098 | 8 | BCAM |
| KLF4 | 0.673 | 0.537470215 | 0.346 | 0.669 | 0.304 | 8 | KLF4 |
| NUCKS1 | 0.673 | 0.49055558 | 0.346 | 0.718 | 0.363 | 8 | NUCKS1 |
| SLC6A6 | 0.67 | 0.670500881 | 0.34 | 0.525 | 0.172 | 8 | SLC6A6 |
| DUSP13 | 0.67 | 0.457300982 | 0.34 | 0.921 | 0.717 | 8 | DUSP1 |
| CD95 | 0.67 | 0.335982442 | 0.34 | 0.894 | 0.58 | 8 | CD9 |
| MT1E1 | 0.669 | 0.539469523 | 0.338 | 0.587 | 0.228 | 8 | MT1E |
| RPL4 | 0.668 | 0.379380253 | 0.336 | 0.92 | 0.719 | 8 | RPL4 |
| HSPB14 | 0.667 | 0.451434974 | 0.334 | 0.786 | 0.455 | 8 | HSPB1 |
| TACSTD27 | 0.667 | 0.33920039 | 0.334 | 0.864 | 0.499 | 8 | TACSTD2 |
| ZFP36L2 | 0.666 | 0.495135964 | 0.332 | 0.71 | 0.362 | 8 | ZFP36L2 |
| NCL | 0.664 | 0.43607522 | 0.328 | 0.773 | 0.437 | 8 | NCL |
| RPS211 | 0.664 | 0.304061934 | 0.328 | 0.985 | 0.89 | 8 | RPS21 |
| RPL51 | 0.663 | 0.351420779 | 0.326 | 0.943 | 0.781 | 8 | RPL5 |
| ITGA2 | 0.662 | 0.701053539 | 0.324 | 0.417 | 0.085 | 8 | ITGA2 |
| MYC | 0.662 | 0.628131185 | 0.324 | 0.494 | 0.155 | 8 | MYC |
| LDHB | 0.662 | 0.46195877 | 0.324 | 0.621 | 0.274 | 8 | LDHB |
| AHNAK | 0.662 | 0.420310197 | 0.324 | 0.711 | 0.357 | 8 | AHNAK |
| NFIB | 0.661 | 0.599705923 | 0.322 | 0.517 | 0.185 | 8 | NFIB |
| TOB11 | 0.661 | 0.50983631 | 0.322 | 0.657 | 0.323 | 8 | TOB1 |
| CAST | 0.659 | 0.471482519 | 0.318 | 0.663 | 0.327 | 8 | CAST |
| TNC | 0.659 | 0.468507856 | 0.318 | 0.525 | 0.176 | 8 | TNC |
| LMO4 | 0.656 | 0.503242173 | 0.312 | 0.602 | 0.28 | 8 | LMO4 |
| ADIRF1 | 0.656 | 0.358946221 | 0.312 | 0.681 | 0.33 | 8 | ADIRF |
| RPL121 | 0.656 | 0.316954945 | 0.312 | 0.949 | 0.786 | 8 | RPL12 |
| RND3 | 0.652 | 0.565098268 | 0.304 | 0.5 | 0.181 | 8 | RND3 |
| HMGN3 | 0.652 | 0.455920514 | 0.304 | 0.556 | 0.235 | 8 | HMGN3 |
| CLSTN1 | 0.651 | 0.607271398 | 0.302 | 0.444 | 0.132 | 8 | CLSTN1 |
| ERRFI1 | 0.651 | 0.577816203 | 0.302 | 0.519 | 0.207 | 8 | ERRFI1 |
| PDLIM1 | 0.651 | 0.528050249 | 0.302 | 0.501 | 0.188 | 8 | PDLIM1 |
| TMSB4X5 | 0.882 | 1.36875443 | 0.764 | 0.966 | 0.682 | 9 | TMSB4X |
| RPS29 | 0.857 | 1.093686931 | 0.714 | 0.977 | 0.913 | 9 | RPS29 |
| RPS27A1 | 0.857 | 0.992506021 | 0.714 | 0.98 | 0.899 | 9 | RPS27A |
| CD52 | 0.844 | 2.34856342 | 0.688 | 0.741 | 0.101 | 9 | CD52 |
| RPS15A2 | 0.833 | 1.005996509 | 0.666 | 0.958 | 0.854 | 9 | RPS15A |
| RPS27 | 0.815 | 1.605520385 | 0.63 | 0.84 | 0.562 | 9 | RPS27 |
| RPS251 | 0.803 | 1.023363478 | 0.606 | 0.919 | 0.783 | 9 | RPS25 |
| CXCR4 | 0.802 | 2.164445578 | 0.604 | 0.669 | 0.127 | 9 | CXCR4 |
| SRGN2 | 0.794 | 1.310008316 | 0.588 | 0.78 | 0.301 | 9 | SRGN |
| PTPRC | 0.786 | 2.180860544 | 0.572 | 0.619 | 0.078 | 9 | PTPRC |
| TRBC2 | 0.775 | 2.449988443 | 0.55 | 0.566 | 0.03 | 9 | TRBC2 |
| RPL13A1 | 0.757 | 0.742815409 | 0.514 | 0.951 | 0.884 | 9 | RPL13A |
| RPS201 | 0.755 | 0.729501161 | 0.51 | 0.94 | 0.873 | 9 | RPS20 |
| HLA-B1 | 0.746 | 0.774881454 | 0.492 | 0.904 | 0.819 | 9 | HLA-B |
| IL32 | 0.742 | 2.386972719 | 0.484 | 0.496 | 0.02 | 9 | IL32 |
| CCL5 | 0.735 | 2.916458438 | 0.47 | 0.496 | 0.044 | 9 | CCL5 |
| RPL23A | 0.734 | 1.127433503 | 0.468 | 0.732 | 0.573 | 9 | RPL23A |
| CD2 | 0.733 | 2.288910647 | 0.466 | 0.478 | 0.018 | 9 | CD2 |
| RPS32 | 0.73 | 0.728807482 | 0.46 | 0.874 | 0.807 | 9 | RPS3 |
| RPL37 | 0.719 | 0.74023239 | 0.438 | 0.847 | 0.792 | 9 | RPL37 |
| CD3D | 0.709 | 2.148857963 | 0.418 | 0.434 | 0.025 | 9 | CD3D |
| IL7R | 0.705 | 2.30458089 | 0.41 | 0.424 | 0.019 | 9 | IL7R |
| HLA-E1 | 0.703 | 0.799451833 | 0.406 | 0.75 | 0.605 | 9 | HLA-E |
| PFN1 | 0.694 | 1.162486792 | 0.388 | 0.618 | 0.422 | 9 | PFN1 |
| ARHGDIB | 0.692 | 1.65560101 | 0.384 | 0.475 | 0.143 | 9 | ARHGDIB |
| RPL39 | 0.688 | 0.789038031 | 0.376 | 0.749 | 0.671 | 9 | RPL39 |
| TSC22D31 | 0.686 | 1.045913707 | 0.372 | 0.624 | 0.401 | 9 | TSC22D3 |
| PTPRCAP | 0.684 | 1.792834833 | 0.368 | 0.404 | 0.051 | 9 | PTPRCAP |
| CD69 | 0.682 | 1.406617452 | 0.364 | 0.429 | 0.076 | 9 | CD69 |
| RGS11 | 0.681 | 1.149414601 | 0.362 | 0.52 | 0.188 | 9 | RGS1 |
| RPL14 | 0.681 | 0.707887416 | 0.362 | 0.754 | 0.704 | 9 | RPL14 |
| RPL10 | 0.678 | 0.79204469 | 0.356 | 0.733 | 0.627 | 9 | RPL10 |
| HLA-C | 0.678 | 0.711491512 | 0.356 | 0.745 | 0.666 | 9 | HLA-C |
| S100A41 | 0.676 | 1.001515598 | 0.352 | 0.595 | 0.349 | 9 | S100A4 |
| HLA-A | 0.676 | 0.812220221 | 0.352 | 0.69 | 0.584 | 9 | HLA-A |
| TNFAIP3 | 0.673 | 1.434709041 | 0.346 | 0.457 | 0.151 | 9 | TNFAIP3 |
| LCP1 | 0.668 | 1.620621472 | 0.336 | 0.381 | 0.056 | 9 | LCP1 |
| UBA52 | 0.668 | 0.738760177 | 0.336 | 0.695 | 0.631 | 9 | UBA52 |
| ETS1 | 0.662 | 1.614149911 | 0.324 | 0.37 | 0.064 | 9 | ETS1 |
| KLRB1 | 0.654 | 2.222701128 | 0.308 | 0.32 | 0.017 | 9 | KLRB1 |
| GZMA | 0.653 | 2.082556817 | 0.306 | 0.317 | 0.016 | 9 | GZMA |
| IGLC7 | 0.98 | 4.590194979 | 0.96 | 0.971 | 0.063 | 10 | IGLC7 |
| IGLL1 | 0.963 | 4.156044318 | 0.926 | 0.937 | 0.033 | 10 | IGLL1 |
| IGLC3 | 0.956 | 4.595624903 | 0.912 | 0.925 | 0.039 | 10 | IGLC3 |
| SSR43 | 0.928 | 1.65167692 | 0.856 | 0.985 | 0.561 | 10 | SSR4 |
| IGJ5 | 0.927 | 2.144494168 | 0.854 | 0.979 | 0.473 | 10 | IGJ |
| MZB11 | 0.906 | 1.967251362 | 0.812 | 0.914 | 0.139 | 10 | MZB1 |
| IGHA15 | 0.867 | 2.20770413 | 0.734 | 0.951 | 0.544 | 10 | IGHA1 |
| GUSBP11 | 0.852 | 3.060556277 | 0.704 | 0.713 | 0.014 | 10 | GUSBP11 |
| SEC11C1 | 0.848 | 1.440596931 | 0.696 | 0.88 | 0.311 | 10 | SEC11C |
| ENAM1 | 0.84 | 1.994561738 | 0.68 | 0.768 | 0.1 | 10 | ENAM |
| HSP90B11 | 0.838 | 1.283343032 | 0.676 | 0.929 | 0.573 | 10 | HSP90B1 |
| DERL31 | 0.825 | 1.700523602 | 0.65 | 0.729 | 0.088 | 10 | DERL3 |
| IGLC2 | 0.815 | 4.951103768 | 0.63 | 0.652 | 0.038 | 10 | IGLC2 |
| IGHA21 | 0.802 | 1.795921046 | 0.604 | 0.763 | 0.225 | 10 | IGHA2 |
| FKBP111 | 0.802 | 1.455748364 | 0.604 | 0.708 | 0.123 | 10 | FKBP11 |
| HERPUD11 | 0.799 | 1.258991546 | 0.598 | 0.816 | 0.321 | 10 | HERPUD1 |
| RGS12 | 0.777 | 1.251810388 | 0.554 | 0.726 | 0.179 | 10 | RGS1 |
| PRDX41 | 0.771 | 1.207834565 | 0.542 | 0.735 | 0.27 | 10 | PRDX4 |
| IGHG31 | 0.764 | 1.931110318 | 0.528 | 0.692 | 0.209 | 10 | IGHG3 |
| IGHG11 | 0.756 | 1.915887793 | 0.512 | 0.668 | 0.194 | 10 | IGHG1 |
| SSR31 | 0.744 | 1.011250818 | 0.488 | 0.719 | 0.293 | 10 | SSR3 |
| SPCS31 | 0.742 | 0.928502333 | 0.484 | 0.751 | 0.341 | 10 | SPCS3 |
| TSC22D32 | 0.73 | 0.896886209 | 0.46 | 0.775 | 0.394 | 10 | TSC22D3 |
| CD79A1 | 0.721 | 1.276658856 | 0.442 | 0.498 | 0.056 | 10 | CD79A |
| SLAMF71 | 0.72 | 1.198456249 | 0.44 | 0.511 | 0.068 | 10 | SLAMF7 |
| IGHG21 | 0.718 | 1.923316655 | 0.436 | 0.534 | 0.111 | 10 | IGHG2 |
| PIM21 | 0.715 | 1.143093759 | 0.43 | 0.514 | 0.084 | 10 | PIM2 |
| FAM46C1 | 0.715 | 1.061082619 | 0.43 | 0.56 | 0.142 | 10 | FAM46C |
| ITM2C1 | 0.713 | 1.084209174 | 0.426 | 0.558 | 0.151 | 10 | ITM2C |
| SPCS11 | 0.713 | 0.811332083 | 0.426 | 0.705 | 0.339 | 10 | SPCS1 |
| IGHG41 | 0.709 | 1.616351803 | 0.418 | 0.615 | 0.23 | 10 | IGHG4 |
| PRDM11 | 0.691 | 1.071144575 | 0.382 | 0.461 | 0.076 | 10 | PRDM1 |
| ERLEC11 | 0.689 | 0.838770674 | 0.378 | 0.595 | 0.246 | 10 | ERLEC1 |
| C19orf101 | 0.688 | 0.906728841 | 0.376 | 0.587 | 0.247 | 10 | C19orf10 |
| XBP17 | 0.688 | 0.616766718 | 0.376 | 0.78 | 0.448 | 10 | XBP1 |
| SELK1 | 0.684 | 0.682682962 | 0.368 | 0.671 | 0.341 | 10 | SELK |
| SEL1L1 | 0.681 | 0.910772691 | 0.362 | 0.509 | 0.156 | 10 | SEL1L |
| IGLL5 | 0.678 | 4.098290804 | 0.356 | 0.397 | 0.049 | 10 | IGLL5 |
| FKBP21 | 0.677 | 0.808040608 | 0.354 | 0.561 | 0.232 | 10 | FKBP2 |
| UBE2J11 | 0.675 | 0.946965629 | 0.35 | 0.49 | 0.154 | 10 | UBE2J1 |
| FCRL51 | 0.674 | 1.106451041 | 0.348 | 0.398 | 0.048 | 10 | FCRL5 |
| TRAM11 | 0.673 | 0.685367977 | 0.346 | 0.645 | 0.34 | 10 | TRAM1 |
| DNAJB91 | 0.671 | 0.873477698 | 0.342 | 0.466 | 0.131 | 10 | DNAJB9 |
| VIMP1 | 0.67 | 0.765659623 | 0.34 | 0.585 | 0.275 | 10 | VIMP |
| SUB11 | 0.67 | 0.682776937 | 0.34 | 0.634 | 0.33 | 10 | SUB1 |
| IGLV6-57 | 0.669 | 3.979010495 | 0.338 | 0.366 | 0.032 | 10 | IGLV6-57 |
| HSPA5 | 0.669 | 0.696465475 | 0.338 | 0.733 | 0.468 | 10 | HSPA5 |
| SDF2L11 | 0.665 | 0.94877522 | 0.33 | 0.424 | 0.099 | 10 | SDF2L1 |
| CYBA1 | 0.664 | 0.722313836 | 0.328 | 0.51 | 0.187 | 10 | CYBA |
| SPCS21 | 0.661 | 0.760663203 | 0.322 | 0.491 | 0.181 | 10 | SPCS2 |
| ISG201 | 0.659 | 0.827745628 | 0.318 | 0.437 | 0.123 | 10 | ISG20 |
| RPN2 | 0.658 | 0.681239994 | 0.316 | 0.571 | 0.284 | 10 | RPN2 |
| RRBP11 | 0.657 | 0.717599415 | 0.314 | 0.587 | 0.303 | 10 | RRBP1 |
| IGLJ3 | 0.654 | 3.454385274 | 0.308 | 0.314 | 0.008 | 10 | IGLJ3 |
| PPIB1 | 0.654 | 0.665761221 | 0.308 | 0.649 | 0.385 | 10 | PPIB |
| IGHM | 0.653 | 1.6300273 | 0.306 | 0.402 | 0.098 | 10 | IGHM |
| MANF | 0.653 | 0.823948169 | 0.306 | 0.434 | 0.137 | 10 | MANF |
| RABAC1 | 0.652 | 0.643331741 | 0.304 | 0.504 | 0.209 | 10 | RABAC1 |
| HLA-DRA1 | 0.979 | 3.409297637 | 0.958 | 0.974 | 0.234 | 11 | HLA-DRA |
| CD743 | 0.972 | 2.730993363 | 0.944 | 0.978 | 0.434 | 11 | CD74 |
| HLA-DRB11 | 0.957 | 3.098287964 | 0.914 | 0.94 | 0.176 | 11 | HLA-DRB1 |
| SRGN3 | 0.94 | 2.229223606 | 0.88 | 0.963 | 0.293 | 11 | SRGN |
| HLA-DPA11 | 0.92 | 3.144408715 | 0.84 | 0.867 | 0.113 | 11 | HLA-DPA1 |
| HLA-DPB1 | 0.92 | 3.125450022 | 0.84 | 0.862 | 0.084 | 11 | HLA-DPB1 |
| TMSB4X6 | 0.894 | 1.370783383 | 0.788 | 0.98 | 0.682 | 11 | TMSB4X |
| FTH12 | 0.893 | 1.502281423 | 0.786 | 0.964 | 0.719 | 11 | FTH1 |
| AIF1 | 0.87 | 2.713790078 | 0.74 | 0.746 | 0.01 | 11 | AIF1 |
| TMSB104 | 0.867 | 1.173838752 | 0.734 | 0.969 | 0.813 | 11 | TMSB10 |
| TYROBP | 0.866 | 2.504740465 | 0.732 | 0.748 | 0.027 | 11 | TYROBP |
| FTL2 | 0.857 | 1.640934596 | 0.714 | 0.972 | 0.82 | 11 | FTL |
| CST33 | 0.855 | 1.873941929 | 0.71 | 0.846 | 0.409 | 11 | CST3 |
| GPR183 | 0.852 | 2.523899117 | 0.704 | 0.735 | 0.052 | 11 | GPR183 |
| HLA-DQA1 | 0.848 | 2.833858683 | 0.696 | 0.715 | 0.043 | 11 | HLA-DQA1 |
| IFI30 | 0.843 | 2.559389807 | 0.686 | 0.719 | 0.072 | 11 | IFI30 |
| HLA-DRB5 | 0.842 | 2.481176892 | 0.684 | 0.723 | 0.082 | 11 | HLA-DRB5 |
| FGL2 | 0.821 | 2.390838145 | 0.642 | 0.654 | 0.02 | 11 | FGL2 |
| ACTB3 | 0.811 | 1.328465774 | 0.622 | 0.862 | 0.553 | 11 | ACTB |
| CTSS | 0.807 | 2.015572961 | 0.614 | 0.716 | 0.218 | 11 | CTSS |
| IL8 | 0.802 | 2.807491118 | 0.604 | 0.673 | 0.123 | 11 | IL8 |
| PLAUR | 0.8 | 2.341950654 | 0.6 | 0.634 | 0.061 | 11 | PLAUR |
| LAPTM5 | 0.798 | 1.83429645 | 0.596 | 0.646 | 0.059 | 11 | LAPTM5 |
| HLA-DQB1 | 0.797 | 2.219999989 | 0.594 | 0.615 | 0.035 | 11 | HLA-DQB1 |
| PSAP1 | 0.797 | 1.468605731 | 0.594 | 0.814 | 0.515 | 11 | PSAP |
| MS4A6A | 0.794 | 2.404135572 | 0.588 | 0.594 | 0.008 | 11 | MS4A6A |
| FCER1G | 0.79 | 2.000649424 | 0.58 | 0.596 | 0.02 | 11 | FCER1G |
| NFKBIA | 0.783 | 1.324320748 | 0.566 | 0.815 | 0.496 | 11 | NFKBIA |
| COTL1 | 0.779 | 1.968054893 | 0.558 | 0.598 | 0.052 | 11 | COTL1 |
| DUSP2 | 0.775 | 1.807582681 | 0.55 | 0.661 | 0.181 | 11 | DUSP2 |
| HLA-DMA | 0.77 | 1.875303009 | 0.54 | 0.594 | 0.09 | 11 | HLA-DMA |
| IL1B | 0.769 | 2.883956522 | 0.538 | 0.547 | 0.017 | 11 | IL1B |
| CPVL | 0.765 | 2.142352324 | 0.53 | 0.54 | 0.015 | 11 | CPVL |
| MNDA | 0.764 | 2.276053244 | 0.528 | 0.533 | 0.006 | 11 | MNDA |
| NAMPT | 0.759 | 1.600624224 | 0.518 | 0.636 | 0.186 | 11 | NAMPT |
| VIM4 | 0.755 | 0.94300776 | 0.51 | 0.783 | 0.335 | 11 | VIM |
| CD83 | 0.75 | 1.895606644 | 0.5 | 0.519 | 0.026 | 11 | CD83 |
| RGS2 | 0.75 | 1.62189148 | 0.5 | 0.614 | 0.153 | 11 | RGS2 |
| PTPRC1 | 0.742 | 1.403496651 | 0.484 | 0.561 | 0.081 | 11 | PTPRC |
| ITGB2 | 0.739 | 1.71166066 | 0.478 | 0.499 | 0.024 | 11 | ITGB2 |
| SH3BGRL3 | 0.737 | 1.357456358 | 0.474 | 0.628 | 0.241 | 11 | SH3BGRL3 |
| PLEK | 0.736 | 1.913215749 | 0.472 | 0.491 | 0.023 | 11 | PLEK |
| LST1 | 0.73 | 1.871565173 | 0.46 | 0.469 | 0.01 | 11 | LST1 |
| TNFAIP31 | 0.729 | 1.489110119 | 0.458 | 0.572 | 0.147 | 11 | TNFAIP3 |
| OAZ1 | 0.728 | 0.909847476 | 0.456 | 0.757 | 0.513 | 11 | OAZ1 |
| BCL2A1 | 0.723 | 2.370264294 | 0.446 | 0.472 | 0.033 | 11 | BCL2A1 |
| HLA-DMB | 0.722 | 1.708100824 | 0.444 | 0.461 | 0.025 | 11 | HLA-DMB |
| CLEC10A | 0.72 | 1.975407268 | 0.44 | 0.443 | 0.004 | 11 | CLEC10A |
| GPX1 | 0.717 | 1.135484633 | 0.434 | 0.635 | 0.314 | 11 | GPX1 |
| LCP11 | 0.716 | 1.506287569 | 0.432 | 0.48 | 0.052 | 11 | LCP1 |
| F13A1 | 0.713 | 2.300211863 | 0.426 | 0.433 | 0.009 | 11 | F13A1 |
| NPC2 | 0.713 | 1.189597124 | 0.426 | 0.63 | 0.321 | 11 | NPC2 |
| TPM31 | 0.705 | 1.075126273 | 0.41 | 0.613 | 0.281 | 11 | TPM3 |
| CCL3 | 0.704 | 2.35809659 | 0.408 | 0.436 | 0.035 | 11 | CCL3 |
| AMICA1 | 0.704 | 1.697703362 | 0.408 | 0.423 | 0.016 | 11 | AMICA1 |
| PFN11 | 0.704 | 0.928505368 | 0.408 | 0.683 | 0.42 | 11 | PFN1 |
| HLA-B2 | 0.704 | 0.524099598 | 0.408 | 0.915 | 0.819 | 11 | HLA-B |
| SAMSN1 | 0.701 | 1.692756775 | 0.402 | 0.457 | 0.06 | 11 | SAMSN1 |
| ZNF331 | 0.7 | 1.529026978 | 0.4 | 0.472 | 0.091 | 11 | ZNF331 |
| ARPC1B | 0.7 | 1.224039423 | 0.4 | 0.53 | 0.175 | 11 | ARPC1B |
| CYBB | 0.698 | 1.794292592 | 0.396 | 0.403 | 0.008 | 11 | CYBB |
| NR4A2 | 0.694 | 1.221356272 | 0.388 | 0.529 | 0.182 | 11 | NR4A2 |
| PPP1R15A1 | 0.693 | 0.833278767 | 0.386 | 0.757 | 0.554 | 11 | PPP1R15A |
| ARPC5 | 0.691 | 1.09913244 | 0.382 | 0.572 | 0.263 | 11 | ARPC5 |
| ARPC2 | 0.691 | 0.850240378 | 0.382 | 0.676 | 0.416 | 11 | ARPC2 |
| RGS13 | 0.69 | 1.198801943 | 0.38 | 0.546 | 0.188 | 11 | RGS1 |
| ARHGDIB1 | 0.687 | 1.057679421 | 0.374 | 0.496 | 0.143 | 11 | ARHGDIB |
| HLA-E2 | 0.686 | 0.590090269 | 0.372 | 0.805 | 0.603 | 11 | HLA-E |
| CTSH | 0.684 | 1.164678724 | 0.368 | 0.522 | 0.213 | 11 | CTSH |
| CD68 | 0.678 | 1.567568987 | 0.356 | 0.365 | 0.012 | 11 | CD68 |
| CTSB5 | 0.677 | 1.133406819 | 0.354 | 0.686 | 0.477 | 11 | CTSB |
| CD14 | 0.675 | 1.755416715 | 0.35 | 0.377 | 0.037 | 11 | CD14 |
| ACTR2 | 0.674 | 1.018941291 | 0.348 | 0.533 | 0.251 | 11 | ACTR2 |
| IGSF6 | 0.673 | 1.597524381 | 0.346 | 0.348 | 0.002 | 11 | IGSF6 |
| ARPC3 | 0.671 | 0.846135172 | 0.342 | 0.578 | 0.312 | 11 | ARPC3 |
| PTPRE | 0.669 | 1.452599884 | 0.338 | 0.366 | 0.034 | 11 | PTPRE |
| CFL1 | 0.668 | 0.744077314 | 0.336 | 0.634 | 0.414 | 11 | CFL1 |
| ATP5E | 0.668 | 0.596740401 | 0.336 | 0.739 | 0.576 | 11 | ATP5E |
| CLEC7A | 0.664 | 1.435290676 | 0.328 | 0.337 | 0.011 | 11 | CLEC7A |
| CD521 | 0.662 | 1.188320875 | 0.324 | 0.43 | 0.116 | 11 | CD52 |
| MS4A7 | 0.658 | 1.587605662 | 0.316 | 0.319 | 0.003 | 11 | MS4A7 |
| GRB2 | 0.658 | 0.958895667 | 0.316 | 0.49 | 0.219 | 11 | GRB2 |
| SAMHD1 | 0.655 | 1.22005547 | 0.31 | 0.391 | 0.099 | 11 | SAMHD1 |
| C5AR1 | 0.654 | 1.544241551 | 0.308 | 0.321 | 0.016 | 11 | C5AR1 |
| S100A42 | 0.654 | 0.715823609 | 0.308 | 0.603 | 0.349 | 11 | S100A4 |
| CTSC1 | 0.653 | 1.157524491 | 0.306 | 0.52 | 0.282 | 11 | CTSC |
| CXCL2 | 0.652 | 1.489043463 | 0.304 | 0.443 | 0.18 | 11 | CXCL2 |
| AREG | 0.652 | 1.459412536 | 0.304 | 0.356 | 0.063 | 11 | AREG |
| SOD2 | 0.651 | 1.198285858 | 0.302 | 0.493 | 0.257 | 11 | SOD2 |
| FCGRT | 0.651 | 1.017760726 | 0.302 | 0.435 | 0.168 | 11 | FCGRT |
| S100A24 | 0.897 | 1.657974982 | 0.794 | 0.969 | 0.357 | 12 | S100A2 |
| POSTN6 | 0.889 | 1.393492867 | 0.778 | 0.991 | 0.427 | 12 | POSTN |
| ALOX155 | 0.849 | 1.280722806 | 0.698 | 0.935 | 0.375 | 12 | ALOX15 |
| KRT52 | 0.845 | 1.32223958 | 0.69 | 0.887 | 0.24 | 12 | KRT5 |
| KRT152 | 0.831 | 1.406968315 | 0.662 | 0.834 | 0.206 | 12 | KRT15 |
| MMP101 | 0.827 | 1.743007578 | 0.654 | 0.787 | 0.186 | 12 | MMP10 |
| CD99 | 0.805 | 0.861304145 | 0.61 | 0.958 | 0.58 | 12 | CD9 |
| TACSTD211 | 0.795 | 0.924448557 | 0.59 | 0.94 | 0.498 | 12 | TACSTD2 |
| KRT171 | 0.786 | 1.321740747 | 0.572 | 0.7 | 0.14 | 12 | KRT17 |
| ETS23 | 0.77 | 0.957292017 | 0.54 | 0.815 | 0.344 | 12 | ETS2 |
| TNC1 | 0.769 | 1.153579359 | 0.538 | 0.699 | 0.171 | 12 | TNC |
| PTHLH1 | 0.762 | 1.184737885 | 0.524 | 0.665 | 0.134 | 12 | PTHLH |
| PERP10 | 0.755 | 0.696107415 | 0.51 | 0.922 | 0.483 | 12 | PERP |
| MIR205HG2 | 0.754 | 0.991154629 | 0.508 | 0.685 | 0.182 | 12 | MIR205HG |
| SFN | 0.744 | 1.102667609 | 0.488 | 0.63 | 0.15 | 12 | SFN |
| SERPINF1 | 0.741 | 0.861669808 | 0.482 | 0.696 | 0.213 | 12 | SERPINF1 |
| F38 | 0.739 | 0.748428253 | 0.478 | 0.841 | 0.411 | 12 | F3 |
| AQP39 | 0.739 | 0.651154685 | 0.478 | 0.887 | 0.447 | 12 | AQP3 |
| NCOA72 | 0.733 | 0.826659072 | 0.466 | 0.798 | 0.39 | 12 | NCOA7 |
| BTF32 | 0.733 | 0.633463698 | 0.466 | 0.892 | 0.572 | 12 | BTF3 |
| CD44 | 0.732 | 0.818242733 | 0.464 | 0.719 | 0.273 | 12 | CD44 |
| LAMB3 | 0.727 | 1.089836008 | 0.454 | 0.56 | 0.107 | 12 | LAMB3 |
| NTRK2 | 0.725 | 1.211035726 | 0.45 | 0.519 | 0.068 | 12 | NTRK2 |
| DST1 | 0.724 | 0.891981739 | 0.448 | 0.649 | 0.205 | 12 | DST |
| FXYD38 | 0.723 | 0.600868319 | 0.446 | 0.898 | 0.511 | 12 | FXYD3 |
| IL332 | 0.721 | 0.926492636 | 0.442 | 0.646 | 0.215 | 12 | IL33 |
| GPX4 | 0.718 | 0.670369809 | 0.436 | 0.836 | 0.495 | 12 | GPX4 |
| TXNDC17 | 0.713 | 0.863606781 | 0.426 | 0.651 | 0.259 | 12 | TXNDC17 |
| SGK1 | 0.713 | 0.831653689 | 0.426 | 0.73 | 0.346 | 12 | SGK1 |
| GAPDH3 | 0.713 | 0.559118711 | 0.426 | 0.919 | 0.638 | 12 | GAPDH |
| EGR17 | 0.711 | 0.558741498 | 0.422 | 0.921 | 0.62 | 12 | EGR1 |
| JUNB5 | 0.711 | 0.519913041 | 0.422 | 0.948 | 0.735 | 12 | JUNB |
| TP631 | 0.709 | 0.98775502 | 0.418 | 0.52 | 0.096 | 12 | TP63 |
| CBR1 | 0.7 | 0.899148728 | 0.4 | 0.573 | 0.191 | 12 | CBR1 |
| SOCS31 | 0.697 | 0.673859188 | 0.394 | 0.695 | 0.313 | 12 | SOCS3 |
| CYR611 | 0.696 | 0.772226386 | 0.392 | 0.599 | 0.205 | 12 | CYR61 |
| KLF10 | 0.695 | 0.817347617 | 0.39 | 0.587 | 0.213 | 12 | KLF10 |
| RND31 | 0.694 | 0.916796702 | 0.388 | 0.56 | 0.181 | 12 | RND3 |
| CLDN12 | 0.69 | 0.808996852 | 0.38 | 0.563 | 0.182 | 12 | CLDN1 |
| ADIRF4 | 0.689 | 0.606019365 | 0.378 | 0.691 | 0.332 | 12 | ADIRF |
| RPL10A2 | 0.689 | 0.428921615 | 0.378 | 0.922 | 0.705 | 12 | RPL10A |
| PMAIP1 | 0.687 | 0.788878344 | 0.374 | 0.615 | 0.249 | 12 | PMAIP1 |
| EIF11 | 0.687 | 0.389734658 | 0.374 | 0.967 | 0.811 | 12 | EIF1 |
| KRT1911 | 0.685 | 0.389609374 | 0.37 | 0.957 | 0.582 | 12 | KRT19 |
| TMEM123 | 0.684 | 0.561740723 | 0.368 | 0.757 | 0.42 | 12 | TMEM123 |
| HS3ST11 | 0.683 | 0.78459516 | 0.366 | 0.563 | 0.2 | 12 | HS3ST1 |
| SLC6A61 | 0.683 | 0.754159246 | 0.366 | 0.543 | 0.174 | 12 | SLC6A6 |
| MYL12B2 | 0.683 | 0.50459748 | 0.366 | 0.835 | 0.542 | 12 | MYL12B |
| FOS6 | 0.682 | 0.385668242 | 0.364 | 0.99 | 0.883 | 12 | FOS |
| PRNP1 | 0.681 | 0.701770448 | 0.362 | 0.552 | 0.187 | 12 | PRNP |
| ACTG15 | 0.681 | 0.448793512 | 0.362 | 0.927 | 0.679 | 12 | ACTG1 |
| ID16 | 0.68 | 0.535460066 | 0.36 | 0.791 | 0.453 | 12 | ID1 |
| S100A114 | 0.68 | 0.477448984 | 0.36 | 0.845 | 0.543 | 12 | S100A11 |
| BCAM1 | 0.679 | 0.838850798 | 0.358 | 0.462 | 0.1 | 12 | BCAM |
| ANXA26 | 0.679 | 0.497417056 | 0.358 | 0.872 | 0.58 | 12 | ANXA2 |
| RPLP0 | 0.679 | 0.335871469 | 0.358 | 0.985 | 0.892 | 12 | RPLP0 |
| S100A10 | 0.678 | 0.654972898 | 0.356 | 0.651 | 0.309 | 12 | S100A10 |
| RPL15 | 0.678 | 0.327372573 | 0.356 | 0.977 | 0.88 | 12 | RPL15 |
| PKM | 0.677 | 0.582935677 | 0.354 | 0.719 | 0.382 | 12 | PKM |
| RPL13A2 | 0.677 | 0.345061005 | 0.354 | 0.981 | 0.883 | 12 | RPL13A |
| EFNA1 | 0.676 | 0.723561308 | 0.352 | 0.569 | 0.227 | 12 | EFNA1 |
| CD552 | 0.676 | 0.606820052 | 0.352 | 0.713 | 0.395 | 12 | CD55 |
| RPL34 | 0.674 | 0.337058546 | 0.348 | 0.965 | 0.835 | 12 | RPL3 |
| MYC1 | 0.673 | 0.7914605 | 0.346 | 0.504 | 0.157 | 12 | MYC |
| IER35 | 0.673 | 0.525825932 | 0.346 | 0.741 | 0.405 | 12 | IER3 |
| TSC22D15 | 0.672 | 0.57035166 | 0.344 | 0.693 | 0.369 | 12 | TSC22D1 |
| ATF35 | 0.67 | 0.500999805 | 0.34 | 0.804 | 0.479 | 12 | ATF3 |
| CTSC2 | 0.669 | 0.576045276 | 0.338 | 0.613 | 0.278 | 12 | CTSC |
| NFE2L2 | 0.668 | 0.526328815 | 0.336 | 0.65 | 0.311 | 12 | NFE2L2 |
| RASSF61 | 0.667 | 0.797772486 | 0.334 | 0.456 | 0.121 | 12 | RASSF6 |
| ERRFI11 | 0.666 | 0.783270825 | 0.332 | 0.534 | 0.208 | 12 | ERRFI1 |
| IFITM32 | 0.666 | 0.324557172 | 0.332 | 0.719 | 0.35 | 12 | IFITM3 |
| LDHB1 | 0.665 | 0.587638756 | 0.33 | 0.597 | 0.277 | 12 | LDHB |
| RPL8 | 0.665 | 0.356307358 | 0.33 | 0.943 | 0.791 | 12 | RPL8 |
| SERPINB10 | 0.664 | 1.071652032 | 0.328 | 0.356 | 0.027 | 12 | SERPINB10 |
| GSTP15 | 0.664 | 0.384254703 | 0.328 | 0.885 | 0.618 | 12 | GSTP1 |
| LGALS7 | 0.663 | 1.00204155 | 0.326 | 0.384 | 0.056 | 12 | LGALS7 |
| MPZL21 | 0.663 | 0.671516193 | 0.326 | 0.513 | 0.19 | 12 | MPZL2 |
| RPS19 | 0.663 | 0.261705298 | 0.326 | 0.994 | 0.926 | 12 | RPS19 |
| PRSS239 | 0.662 | 0.374749904 | 0.324 | 0.819 | 0.453 | 12 | PRSS23 |
| CEBPD | 0.66 | 0.590694476 | 0.32 | 0.583 | 0.264 | 12 | CEBPD |
| C17orf76-AS13 | 0.66 | 0.459053294 | 0.32 | 0.78 | 0.499 | 12 | C17orf76-AS1 |
| RPS252 | 0.66 | 0.288657942 | 0.32 | 0.943 | 0.783 | 12 | RPS25 |
| RPL41 | 0.658 | 0.316810504 | 0.316 | 0.972 | 0.866 | 12 | RPL41 |
| KRT6A | 0.656 | 1.016809249 | 0.312 | 0.384 | 0.071 | 12 | KRT6A |
| GPR155 | 0.656 | 0.942210762 | 0.312 | 0.365 | 0.055 | 12 | GPR155 |
| FMO2 | 0.656 | 0.748663246 | 0.312 | 0.496 | 0.183 | 12 | FMO2 |
| ADH71 | 0.655 | 0.727793937 | 0.31 | 0.451 | 0.139 | 12 | ADH7 |
| ALDH3A13 | 0.655 | 0.476670958 | 0.31 | 0.577 | 0.245 | 12 | ALDH3A1 |
| SERPINB2 | 0.653 | 1.278231215 | 0.306 | 0.364 | 0.06 | 12 | SERPINB2 |
| SNCA | 0.651 | 0.706255775 | 0.302 | 0.418 | 0.115 | 12 | SNCA |
| SERPINB41 | 0.651 | 0.610786958 | 0.302 | 0.55 | 0.234 | 12 | SERPINB4 |
| ALDH3A21 | 0.651 | 0.590511597 | 0.302 | 0.524 | 0.223 | 12 | ALDH3A2 |
| SERTAD1 | 0.651 | 0.562834565 | 0.302 | 0.567 | 0.271 | 12 | SERTAD1 |
| RPS242 | 0.651 | 0.288344362 | 0.302 | 0.962 | 0.841 | 12 | RPS24 |
| PIGR6 | 0.86 | 1.446278921 | 0.72 | 0.93 | 0.33 | 13 | PIGR |
| SLPI8 | 0.856 | 1.301451544 | 0.712 | 1 | 0.738 | 13 | SLPI |
| LYZ5 | 0.836 | 0.732349816 | 0.672 | 0.908 | 0.329 | 13 | LYZ |
| AZGP11 | 0.826 | 1.845405873 | 0.652 | 0.766 | 0.129 | 13 | AZGP1 |
| BPIFB15 | 0.821 | 0.977783579 | 0.642 | 0.917 | 0.395 | 13 | BPIFB1 |
| TCN11 | 0.803 | 1.602608969 | 0.606 | 0.728 | 0.139 | 13 | TCN1 |
| ZG16B1 | 0.792 | 0.763924561 | 0.584 | 0.775 | 0.22 | 13 | ZG16B |
| STATH8 | 0.79 | 1.010507997 | 0.58 | 0.862 | 0.381 | 13 | STATH |
| LTF1 | 0.782 | 1.35176419 | 0.564 | 0.719 | 0.167 | 13 | LTF |
| BPIFA14 | 0.779 | 0.873976581 | 0.558 | 0.897 | 0.465 | 13 | BPIFA1 |
| SCGB3A11 | 0.741 | 2.66753041 | 0.482 | 0.549 | 0.093 | 13 | SCGB3A1 |
| WFDC28 | 0.738 | 0.99575338 | 0.476 | 0.801 | 0.445 | 13 | WFDC2 |
| PIP1 | 0.726 | 1.045890852 | 0.452 | 0.568 | 0.112 | 13 | PIP |
| MSMB1 | 0.713 | 2.859512155 | 0.426 | 0.575 | 0.206 | 13 | MSMB |
| AGR29 | 0.71 | 1.453180462 | 0.42 | 0.743 | 0.427 | 13 | AGR2 |
| DMBT11 | 0.701 | 1.280762439 | 0.402 | 0.505 | 0.107 | 13 | DMBT1 |
| MT-ND2 | 0.696 | 0.752152467 | 0.392 | 0.818 | 0.578 | 13 | MT-ND2 |
| KRT71 | 0.685 | 0.960468569 | 0.37 | 0.568 | 0.231 | 13 | KRT7 |
| MT-ND3 | 0.681 | 0.69875301 | 0.362 | 0.772 | 0.518 | 13 | MT-ND3 |
| MT-CO32 | 0.677 | 0.592866286 | 0.354 | 0.846 | 0.621 | 13 | MT-CO3 |
| CXCL175 | 0.676 | 0.715758343 | 0.352 | 0.633 | 0.314 | 13 | CXCL17 |
| RP11-1143G9.41 | 0.675 | 0.479298974 | 0.35 | 0.486 | 0.129 | 13 | RP11-1143G9.4 |
| C6orf581 | 0.672 | 0.483367037 | 0.344 | 0.474 | 0.116 | 13 | C6orf58 |
| LCN21 | 0.671 | 1.57750168 | 0.342 | 0.462 | 0.147 | 13 | LCN2 |
| EHF1 | 0.664 | 0.705683992 | 0.328 | 0.578 | 0.282 | 13 | EHF |
| MUC5B | 0.663 | 3.114186053 | 0.326 | 0.359 | 0.045 | 13 | MUC5B |
| FAM3D2 | 0.663 | 0.992149257 | 0.326 | 0.436 | 0.128 | 13 | FAM3D |
| CLU2 | 0.659 | 0.968695068 | 0.318 | 0.59 | 0.32 | 13 | CLU |
| CST34 | 0.657 | 0.597402081 | 0.314 | 0.655 | 0.419 | 13 | CST3 |
| MT-CYB | 0.655 | 0.589532737 | 0.31 | 0.786 | 0.59 | 13 | MT-CYB |
| XBP18 | 0.651 | 0.517211249 | 0.302 | 0.706 | 0.453 | 13 | XBP1 |
| DCN1 | 0.977 | 2.714404223 | 0.954 | 0.998 | 0.131 | 14 | DCN |
| COL1A21 | 0.964 | 2.552187029 | 0.928 | 0.975 | 0.087 | 14 | COL1A2 |
| LUM1 | 0.954 | 2.65153381 | 0.908 | 0.959 | 0.108 | 14 | LUM |
| COL3A11 | 0.936 | 2.501696453 | 0.872 | 0.929 | 0.089 | 14 | COL3A1 |
| MGP1 | 0.927 | 2.514624269 | 0.854 | 0.917 | 0.133 | 14 | MGP |
| LGALS11 | 0.922 | 2.010230268 | 0.844 | 0.936 | 0.218 | 14 | LGALS1 |
| FBLN11 | 0.919 | 2.297849502 | 0.838 | 0.89 | 0.092 | 14 | FBLN1 |
| CALD11 | 0.916 | 2.133352139 | 0.832 | 0.899 | 0.122 | 14 | CALD1 |
| CPE1 | 0.902 | 2.159063315 | 0.804 | 0.864 | 0.083 | 14 | CPE |
| SFRP11 | 0.882 | 2.214701077 | 0.764 | 0.814 | 0.077 | 14 | SFRP1 |
| SPARC2 | 0.88 | 1.857212803 | 0.76 | 0.85 | 0.122 | 14 | SPARC |
| IGFBP72 | 0.872 | 1.621231607 | 0.744 | 0.91 | 0.281 | 14 | IGFBP7 |
| COL1A11 | 0.867 | 2.233080421 | 0.734 | 0.777 | 0.052 | 14 | COL1A1 |
| AEBP11 | 0.864 | 2.094329617 | 0.728 | 0.772 | 0.054 | 14 | AEBP1 |
| VIM5 | 0.86 | 1.306462891 | 0.72 | 0.933 | 0.336 | 14 | VIM |
| POSTN8 | 0.855 | 1.667289934 | 0.71 | 0.913 | 0.437 | 14 | POSTN |
| C1S1 | 0.853 | 1.961412839 | 0.706 | 0.754 | 0.062 | 14 | C1S |
| IFITM33 | 0.852 | 1.303930989 | 0.704 | 0.904 | 0.349 | 14 | IFITM3 |
| PCOLCE1 | 0.847 | 1.895175299 | 0.694 | 0.736 | 0.048 | 14 | PCOLCE |
| SERPING11 | 0.846 | 1.815387913 | 0.692 | 0.752 | 0.077 | 14 | SERPING1 |
| SFRP21 | 0.829 | 2.231000349 | 0.658 | 0.703 | 0.055 | 14 | SFRP2 |
| THY11 | 0.825 | 1.944173599 | 0.65 | 0.685 | 0.037 | 14 | THY1 |
| C1R1 | 0.824 | 1.793220029 | 0.648 | 0.706 | 0.072 | 14 | C1R |
| VCAN | 0.817 | 1.846801598 | 0.634 | 0.685 | 0.055 | 14 | VCAN |
| CRABP21 | 0.815 | 1.946262321 | 0.63 | 0.669 | 0.044 | 14 | CRABP2 |
| FGF7 | 0.805 | 1.954714307 | 0.61 | 0.641 | 0.031 | 14 | FGF7 |
| TPM21 | 0.805 | 1.831781189 | 0.61 | 0.665 | 0.069 | 14 | TPM2 |
| TAGLN1 | 0.801 | 2.050468361 | 0.602 | 0.658 | 0.07 | 14 | TAGLN |
| IGFBP61 | 0.799 | 1.842942198 | 0.598 | 0.642 | 0.05 | 14 | IGFBP6 |
| SEPP11 | 0.797 | 1.344994826 | 0.594 | 0.786 | 0.295 | 14 | SEPP1 |
| CDH111 | 0.794 | 1.750302985 | 0.588 | 0.632 | 0.049 | 14 | CDH11 |
| RARRES21 | 0.793 | 1.676058258 | 0.586 | 0.632 | 0.052 | 14 | RARRES2 |
| SELM1 | 0.792 | 1.203085328 | 0.584 | 0.773 | 0.291 | 14 | SELM |
| COL6A2 | 0.791 | 1.823523278 | 0.582 | 0.614 | 0.036 | 14 | COL6A2 |
| NNMT | 0.79 | 1.827647401 | 0.58 | 0.635 | 0.066 | 14 | NNMT |
| PDGFRA | 0.79 | 1.783826075 | 0.58 | 0.607 | 0.027 | 14 | PDGFRA |
| PRRX11 | 0.79 | 1.662283909 | 0.58 | 0.619 | 0.039 | 14 | PRRX1 |
| BGN | 0.787 | 1.629505729 | 0.574 | 0.609 | 0.032 | 14 | BGN |
| PPAP2B1 | 0.786 | 1.622611903 | 0.572 | 0.664 | 0.126 | 14 | PPAP2B |
| CLDN111 | 0.785 | 1.825410327 | 0.57 | 0.605 | 0.038 | 14 | CLDN11 |
| CXCL141 | 0.781 | 2.07704742 | 0.562 | 0.604 | 0.048 | 14 | CXCL14 |
| MXRA8 | 0.78 | 1.679099284 | 0.56 | 0.589 | 0.029 | 14 | MXRA8 |
| LIMA1 | 0.778 | 1.266450116 | 0.556 | 0.738 | 0.285 | 14 | LIMA1 |
| GLIPR1 | 0.771 | 1.563442396 | 0.542 | 0.605 | 0.07 | 14 | GLIPR1 |
| IGFBP4 | 0.771 | 1.505237906 | 0.542 | 0.618 | 0.093 | 14 | IGFBP4 |
| COL8A1 | 0.77 | 1.846509435 | 0.54 | 0.563 | 0.023 | 14 | COL8A1 |
| TPM11 | 0.768 | 1.617071729 | 0.536 | 0.634 | 0.135 | 14 | TPM1 |
| TMSB4X8 | 0.766 | 0.662848956 | 0.532 | 0.956 | 0.687 | 14 | TMSB4X |
| FSTL1 | 0.765 | 1.504147162 | 0.53 | 0.596 | 0.079 | 14 | FSTL1 |
| LAPTM4A | 0.763 | 0.991981814 | 0.526 | 0.777 | 0.379 | 14 | LAPTM4A |
| APOD | 0.762 | 1.790599836 | 0.524 | 0.582 | 0.072 | 14 | APOD |
| LAMP5 | 0.757 | 1.724791575 | 0.514 | 0.547 | 0.035 | 14 | LAMP5 |
| COL15A1 | 0.757 | 1.549056157 | 0.514 | 0.563 | 0.053 | 14 | COL15A1 |
| KCNE4 | 0.756 | 1.80333644 | 0.512 | 0.538 | 0.024 | 14 | KCNE4 |
| THBS1 | 0.755 | 1.616544786 | 0.51 | 0.589 | 0.091 | 14 | THBS1 |
| ITGB1 | 0.753 | 1.197183059 | 0.506 | 0.667 | 0.222 | 14 | ITGB1 |
| ITGBL1 | 0.749 | 1.746203511 | 0.498 | 0.52 | 0.022 | 14 | ITGBL1 |
| IGF2 | 0.748 | 1.75527327 | 0.496 | 0.522 | 0.024 | 14 | IGF2 |
| MFAP4 | 0.743 | 1.563340982 | 0.486 | 0.515 | 0.029 | 14 | MFAP4 |
| COL6A1 | 0.743 | 1.554662858 | 0.486 | 0.545 | 0.069 | 14 | COL6A1 |
| IL6ST1 | 0.741 | 1.054688783 | 0.482 | 0.692 | 0.275 | 14 | IL6ST |
| TGM2 | 0.74 | 1.559205435 | 0.48 | 0.527 | 0.055 | 14 | TGM2 |
| TMEM176B | 0.74 | 1.459565098 | 0.48 | 0.519 | 0.039 | 14 | TMEM176B |
| MYL9 | 0.736 | 1.320281848 | 0.472 | 0.558 | 0.098 | 14 | MYL9 |
| RARRES11 | 0.734 | 1.447574381 | 0.468 | 0.627 | 0.191 | 14 | RARRES1 |
| EID1 | 0.734 | 0.903985212 | 0.468 | 0.756 | 0.423 | 14 | EID1 |
| S100A68 | 0.734 | 0.52229257 | 0.468 | 0.988 | 0.815 | 14 | S100A6 |
| EDIL3 | 0.729 | 1.582230746 | 0.458 | 0.488 | 0.033 | 14 | EDIL3 |
| FN1 | 0.727 | 1.483559244 | 0.454 | 0.506 | 0.056 | 14 | FN1 |
| CLU3 | 0.727 | 1.03558606 | 0.454 | 0.696 | 0.319 | 14 | CLU |
| NBL1 | 0.726 | 1.344928049 | 0.452 | 0.522 | 0.079 | 14 | NBL1 |
| SPON1 | 0.724 | 1.575317981 | 0.448 | 0.467 | 0.02 | 14 | SPON1 |
| APP | 0.722 | 0.99292908 | 0.444 | 0.673 | 0.295 | 14 | APP |
| CCL2 | 0.721 | 1.705598176 | 0.442 | 0.506 | 0.077 | 14 | CCL2 |
| SERPINF11 | 0.721 | 0.966710248 | 0.442 | 0.637 | 0.221 | 14 | SERPINF1 |
| PTRF1 | 0.717 | 1.045721964 | 0.434 | 0.563 | 0.132 | 14 | PTRF |
| GJA1 | 0.716 | 1.450087646 | 0.432 | 0.469 | 0.041 | 14 | GJA1 |
| CFH | 0.715 | 1.074220268 | 0.43 | 0.609 | 0.225 | 14 | CFH |
| INMT | 0.713 | 1.546367309 | 0.426 | 0.448 | 0.022 | 14 | INMT |
| SULF1 | 0.712 | 1.481054675 | 0.424 | 0.464 | 0.044 | 14 | SULF1 |
| PIK3R1 | 0.712 | 0.982527851 | 0.424 | 0.607 | 0.212 | 14 | PIK3R1 |
| CPXM1 | 0.709 | 1.416667637 | 0.418 | 0.45 | 0.032 | 14 | CPXM1 |
| SLC39A6 | 0.707 | 1.329609499 | 0.414 | 0.496 | 0.103 | 14 | SLC39A6 |
| TIMP3 | 0.707 | 1.326465341 | 0.414 | 0.485 | 0.078 | 14 | TIMP3 |
| CNN3 | 0.706 | 0.90753144 | 0.412 | 0.635 | 0.275 | 14 | CNN3 |
| COL6A3 | 0.705 | 1.416451042 | 0.41 | 0.435 | 0.024 | 14 | COL6A3 |
| FGFR1 | 0.704 | 1.263248233 | 0.408 | 0.455 | 0.049 | 14 | FGFR1 |
| TIMP1 | 0.701 | 1.017941219 | 0.402 | 0.596 | 0.243 | 14 | TIMP1 |
| NGFRAP1 | 0.7 | 1.053859761 | 0.4 | 0.52 | 0.143 | 14 | NGFRAP1 |
| TWIST1 | 0.699 | 1.372355211 | 0.398 | 0.418 | 0.019 | 14 | TWIST1 |
| TIMP2 | 0.699 | 1.251259585 | 0.398 | 0.434 | 0.036 | 14 | TIMP2 |
| PGRMC1 | 0.697 | 1.054384613 | 0.394 | 0.535 | 0.171 | 14 | PGRMC1 |
| GUCY1A3 | 0.695 | 1.351394809 | 0.39 | 0.421 | 0.034 | 14 | GUCY1A3 |
| S100A43 | 0.695 | 0.586859137 | 0.39 | 0.724 | 0.349 | 14 | S100A4 |
| ISLR | 0.693 | 1.255586022 | 0.386 | 0.419 | 0.035 | 14 | ISLR |
| EFEMP1 | 0.692 | 1.374268398 | 0.384 | 0.453 | 0.084 | 14 | EFEMP1 |
| LRP1 | 0.692 | 1.153645647 | 0.384 | 0.432 | 0.049 | 14 | LRP1 |
| S100A131 | 0.691 | 0.963532546 | 0.382 | 0.556 | 0.215 | 14 | S100A13 |
| IFITM1 | 0.69 | 0.978299367 | 0.38 | 0.515 | 0.148 | 14 | IFITM1 |
| TPM4 | 0.688 | 0.791713585 | 0.376 | 0.653 | 0.342 | 14 | TPM4 |
| TMEM176A | 0.687 | 1.259098186 | 0.374 | 0.4 | 0.025 | 14 | TMEM176A |
| RGS5 | 0.684 | 1.806335726 | 0.368 | 0.428 | 0.07 | 14 | RGS5 |
| IFITM21 | 0.684 | 0.805805234 | 0.368 | 0.561 | 0.203 | 14 | IFITM2 |
| CTSK | 0.682 | 1.186314479 | 0.364 | 0.391 | 0.027 | 14 | CTSK |
| COLEC12 | 0.68 | 1.274126366 | 0.36 | 0.377 | 0.016 | 14 | COLEC12 |
| GNG111 | 0.68 | 0.841880952 | 0.36 | 0.476 | 0.109 | 14 | GNG11 |
| TBX3 | 0.679 | 1.274316218 | 0.358 | 0.381 | 0.022 | 14 | TBX3 |
| GPX3 | 0.678 | 1.263333037 | 0.356 | 0.423 | 0.081 | 14 | GPX3 |
| FBN1 | 0.677 | 1.243384153 | 0.354 | 0.379 | 0.026 | 14 | FBN1 |
| LAMA4 | 0.677 | 1.141685641 | 0.354 | 0.388 | 0.032 | 14 | LAMA4 |
| 10-Sep | 0.677 | 1.125062013 | 0.354 | 0.416 | 0.069 | 14 | 10-Sep |
| LAMB1 | 0.677 | 1.000662944 | 0.354 | 0.45 | 0.099 | 14 | LAMB1 |
| FRZB | 0.676 | 1.288346595 | 0.352 | 0.382 | 0.031 | 14 | FRZB |
| CRABP1 | 0.675 | 1.546278273 | 0.35 | 0.366 | 0.017 | 14 | CRABP1 |
| ITM2B3 | 0.675 | 0.453057994 | 0.35 | 0.885 | 0.65 | 14 | ITM2B |
| MMP2 | 0.673 | 1.277364683 | 0.346 | 0.365 | 0.018 | 14 | MMP2 |
| MDFIC | 0.673 | 1.069008918 | 0.346 | 0.43 | 0.096 | 14 | MDFIC |
| GATA2 | 0.67 | 1.12919174 | 0.34 | 0.375 | 0.033 | 14 | GATA2 |
| FTL3 | 0.67 | 0.328295491 | 0.34 | 0.938 | 0.824 | 14 | FTL |
| CD631 | 0.669 | 0.489146419 | 0.338 | 0.812 | 0.581 | 14 | CD63 |
| DKK3 | 0.666 | 1.09068044 | 0.332 | 0.373 | 0.042 | 14 | DKK3 |
| CPXM2 | 0.665 | 1.160176158 | 0.33 | 0.372 | 0.043 | 14 | CPXM2 |
| ADAMTS11 | 0.664 | 1.280899682 | 0.328 | 0.389 | 0.064 | 14 | ADAMTS1 |
| PEBP1 | 0.664 | 0.639640895 | 0.328 | 0.688 | 0.435 | 14 | PEBP1 |
| CFI | 0.662 | 1.113910433 | 0.324 | 0.381 | 0.061 | 14 | CFI |
| PSAP2 | 0.66 | 0.453751442 | 0.32 | 0.756 | 0.52 | 14 | PSAP |
| PDGFRB | 0.659 | 1.116460937 | 0.318 | 0.342 | 0.023 | 14 | PDGFRB |
| UBB | 0.658 | 0.41814722 | 0.316 | 0.897 | 0.747 | 14 | UBB |
| MYL61 | 0.658 | 0.409188863 | 0.316 | 0.888 | 0.753 | 14 | MYL6 |
| CD991 | 0.657 | 0.706934617 | 0.314 | 0.51 | 0.216 | 14 | CD99 |
| ENPP2 | 0.656 | 1.106342169 | 0.312 | 0.343 | 0.031 | 14 | ENPP2 |
| NUCKS11 | 0.656 | 0.525869407 | 0.312 | 0.646 | 0.372 | 14 | NUCKS1 |
| WNT5A | 0.654 | 1.171983936 | 0.308 | 0.347 | 0.042 | 14 | WNT5A |
| RAD21 | 0.654 | 0.966403137 | 0.308 | 0.457 | 0.18 | 14 | RAD21 |
| CSRP2 | 0.653 | 1.127310987 | 0.306 | 0.398 | 0.106 | 14 | CSRP2 |
| PAX9 | 0.653 | 1.064887142 | 0.306 | 0.354 | 0.053 | 14 | PAX9 |
| C8orf41 | 0.653 | 0.956593695 | 0.306 | 0.465 | 0.178 | 14 | C8orf4 |
| CTGF | 0.652 | 1.464691666 | 0.304 | 0.405 | 0.117 | 14 | CTGF |
| IGJ6 | 0.842 | 0.686067826 | 0.684 | 0.953 | 0.481 | 15 | IGJ |
| IGHG42 | 0.832 | 1.327242678 | 0.664 | 0.801 | 0.23 | 15 | IGHG4 |
| IGHG12 | 0.775 | 1.202256187 | 0.55 | 0.689 | 0.2 | 15 | IGHG1 |
| POSTN9 | 0.746 | 0.686432516 | 0.492 | 0.853 | 0.439 | 15 | POSTN |
| IGHA16 | 0.739 | 0.382110625 | 0.478 | 0.822 | 0.554 | 15 | IGHA1 |
| IGHG22 | 0.733 | 0.743480915 | 0.466 | 0.585 | 0.116 | 15 | IGHG2 |
| IGHG32 | 0.711 | 0.901923591 | 0.422 | 0.578 | 0.219 | 15 | IGHG3 |
| SSR44 | 0.687 | 0.552703784 | 0.374 | 0.756 | 0.574 | 15 | SSR4 |
| MTRNR2L13 | 0.681 | 0.597755035 | 0.362 | 0.889 | 0.824 | 15 | MTRNR2L1 |
| IGKV3-20 | 0.673 | 1.058655483 | 0.346 | 0.399 | 0.05 | 15 | IGKV3-20 |
| IGHA22 | 0.658 | 0.714383556 | 0.316 | 0.508 | 0.24 | 15 | IGHA2 |
| AC096579.71 | 0.657 | 0.799028525 | 0.314 | 0.402 | 0.092 | 15 | AC096579.7 |
| CAPS | 0.944 | 3.006639471 | 0.888 | 0.91 | 0.135 | 16 | CAPS |
| C9orf24 | 0.909 | 2.886746031 | 0.818 | 0.823 | 0.013 | 16 | C9orf24 |
| TSPAN12 | 0.907 | 1.999627155 | 0.814 | 0.908 | 0.242 | 16 | TSPAN1 |
| PIFO | 0.905 | 2.523694361 | 0.81 | 0.813 | 0.009 | 16 | PIFO |
| TPPP3 | 0.898 | 2.618348084 | 0.796 | 0.801 | 0.012 | 16 | TPPP3 |
| C20orf85 | 0.895 | 2.578291078 | 0.79 | 0.793 | 0.005 | 16 | C20orf85 |
| SNTN | 0.873 | 2.68209007 | 0.746 | 0.749 | 0.006 | 16 | SNTN |
| FAM183A | 0.865 | 2.305782135 | 0.73 | 0.733 | 0.006 | 16 | FAM183A |
| TUBB4B | 0.863 | 1.83007981 | 0.726 | 0.819 | 0.201 | 16 | TUBB4B |
| TUBA1A | 0.862 | 1.972843663 | 0.724 | 0.805 | 0.141 | 16 | TUBA1A |
| GSTA1 | 0.845 | 2.656938347 | 0.69 | 0.711 | 0.042 | 16 | GSTA1 |
| C11orf88 | 0.841 | 2.227525964 | 0.682 | 0.687 | 0.007 | 16 | C11orf88 |
| RSPH1 | 0.841 | 2.14509879 | 0.682 | 0.685 | 0.005 | 16 | RSPH1 |
| PRDX5 | 0.841 | 1.53257466 | 0.682 | 0.841 | 0.383 | 16 | PRDX5 |
| OMG | 0.839 | 2.538421279 | 0.678 | 0.683 | 0.008 | 16 | OMG |
| AGR3 | 0.838 | 2.263658912 | 0.676 | 0.693 | 0.026 | 16 | AGR3 |
| CAPSL | 0.838 | 2.152561498 | 0.676 | 0.679 | 0.003 | 16 | CAPSL |
| CIB1 | 0.838 | 1.588524445 | 0.676 | 0.807 | 0.245 | 16 | CIB1 |
| CCDC170 | 0.835 | 1.982562219 | 0.67 | 0.677 | 0.007 | 16 | CCDC170 |
| DYNLT1 | 0.833 | 1.69790557 | 0.666 | 0.765 | 0.181 | 16 | DYNLT1 |
| HSP90AA1 | 0.832 | 1.236999184 | 0.664 | 0.908 | 0.529 | 16 | HSP90AA1 |
| IFT57 | 0.829 | 1.809975265 | 0.658 | 0.715 | 0.088 | 16 | IFT57 |
| DNAH5 | 0.827 | 2.001240329 | 0.654 | 0.661 | 0.01 | 16 | DNAH5 |
| DYNLL1 | 0.824 | 1.322972668 | 0.648 | 0.839 | 0.321 | 16 | DYNLL1 |
| EZR5 | 0.822 | 1.070906269 | 0.644 | 0.91 | 0.517 | 16 | EZR |
| TMEM190 | 0.82 | 2.24641031 | 0.64 | 0.643 | 0.004 | 16 | TMEM190 |
| C1orf194 | 0.819 | 1.949892022 | 0.638 | 0.641 | 0.005 | 16 | C1orf194 |
| ATPIF1 | 0.819 | 1.373752192 | 0.638 | 0.825 | 0.364 | 16 | ATPIF1 |
| NUCB21 | 0.818 | 1.342116714 | 0.636 | 0.809 | 0.264 | 16 | NUCB2 |
| CALM1 | 0.818 | 1.072286213 | 0.636 | 0.9 | 0.558 | 16 | CALM1 |
| MORN2 | 0.816 | 1.819843299 | 0.632 | 0.657 | 0.035 | 16 | MORN2 |
| RP11-356K23.1 | 0.815 | 2.245614901 | 0.63 | 0.633 | 0.005 | 16 | RP11-356K23.1 |
| PSENEN | 0.815 | 1.788911884 | 0.63 | 0.697 | 0.107 | 16 | PSENEN |
| SPA17 | 0.812 | 1.909801662 | 0.624 | 0.635 | 0.014 | 16 | SPA17 |
| C9orf116 | 0.808 | 1.948005636 | 0.616 | 0.622 | 0.009 | 16 | C9orf116 |
| ZMYND10 | 0.803 | 1.821467324 | 0.606 | 0.608 | 0.004 | 16 | ZMYND10 |
| ROPN1L | 0.801 | 1.845848822 | 0.602 | 0.606 | 0.004 | 16 | ROPN1L |
| CETN2 | 0.801 | 1.704804242 | 0.602 | 0.639 | 0.048 | 16 | CETN2 |
| LRRIQ1 | 0.799 | 1.908444814 | 0.598 | 0.6 | 0.004 | 16 | LRRIQ1 |
| DNAH12 | 0.799 | 1.874337945 | 0.598 | 0.6 | 0.003 | 16 | DNAH12 |
| C5orf49 | 0.796 | 1.80574215 | 0.592 | 0.594 | 0.003 | 16 | C5orf49 |
| PLAC81 | 0.794 | 1.494394492 | 0.588 | 0.675 | 0.113 | 16 | PLAC8 |
| TMC5 | 0.791 | 1.671749329 | 0.582 | 0.629 | 0.067 | 16 | TMC5 |
| GSTP16 | 0.788 | 0.850628029 | 0.576 | 0.936 | 0.621 | 16 | GSTP1 |
| CCDC146 | 0.785 | 1.704630632 | 0.57 | 0.594 | 0.03 | 16 | CCDC146 |
| C1orf173 | 0.784 | 1.74159299 | 0.568 | 0.57 | 0.002 | 16 | C1orf173 |
| CALM22 | 0.783 | 0.890249222 | 0.566 | 0.892 | 0.558 | 16 | CALM2 |
| CYP4B11 | 0.782 | 1.485373937 | 0.564 | 0.679 | 0.151 | 16 | CYP4B1 |
| CHST9 | 0.78 | 1.387294037 | 0.56 | 0.663 | 0.135 | 16 | CHST9 |
| TCTEX1D4 | 0.779 | 1.856297015 | 0.558 | 0.562 | 0.005 | 16 | TCTEX1D4 |
| ARL3 | 0.777 | 1.358946088 | 0.554 | 0.677 | 0.168 | 16 | ARL3 |
| CD591 | 0.776 | 1.097011654 | 0.552 | 0.793 | 0.364 | 16 | CD59 |
| FAM216B | 0.774 | 1.72106614 | 0.548 | 0.55 | 0.002 | 16 | FAM216B |
| SPAG6 | 0.773 | 1.683403161 | 0.546 | 0.548 | 0.002 | 16 | SPAG6 |
| FAM154B | 0.771 | 1.643768798 | 0.542 | 0.546 | 0.004 | 16 | FAM154B |
| FAM81B | 0.769 | 1.682579595 | 0.538 | 0.54 | 0.002 | 16 | FAM81B |
| FAM229B | 0.769 | 1.550779216 | 0.538 | 0.564 | 0.032 | 16 | FAM229B |
| SMIM22 | 0.769 | 1.426371837 | 0.538 | 0.61 | 0.098 | 16 | SMIM22 |
| EFCAB1 | 0.768 | 1.612683225 | 0.536 | 0.54 | 0.006 | 16 | EFCAB1 |
| NQO1 | 0.766 | 1.378767769 | 0.532 | 0.602 | 0.086 | 16 | NQO1 |
| ABCA13 | 0.765 | 1.370732364 | 0.53 | 0.618 | 0.104 | 16 | ABCA13 |
| IK | 0.762 | 1.269952331 | 0.524 | 0.637 | 0.138 | 16 | IK |
| ARMC3 | 0.76 | 1.585896808 | 0.52 | 0.522 | 0.002 | 16 | ARMC3 |
| FOXJ1 | 0.757 | 1.53590316 | 0.514 | 0.52 | 0.007 | 16 | FOXJ1 |
| PRDX13 | 0.757 | 0.868376046 | 0.514 | 0.847 | 0.514 | 16 | PRDX1 |
| CDHR3 | 0.756 | 1.69521917 | 0.512 | 0.516 | 0.006 | 16 | CDHR3 |
| SCGB2A1 | 0.756 | 1.628198924 | 0.512 | 0.56 | 0.058 | 16 | SCGB2A1 |
| IQCG | 0.756 | 1.457287415 | 0.512 | 0.532 | 0.022 | 16 | IQCG |
| ANXA17 | 0.755 | 0.612768055 | 0.51 | 0.968 | 0.685 | 16 | ANXA1 |
| RRAD | 0.754 | 1.576154011 | 0.508 | 0.526 | 0.02 | 16 | RRAD |
| TSPAN19 | 0.753 | 1.698080969 | 0.506 | 0.508 | 0.003 | 16 | TSPAN19 |
| PCM1 | 0.753 | 1.15246056 | 0.506 | 0.671 | 0.202 | 16 | PCM1 |
| FAM92B | 0.752 | 1.499972451 | 0.504 | 0.514 | 0.012 | 16 | FAM92B |
| DYDC2 | 0.75 | 1.625643143 | 0.5 | 0.502 | 0.002 | 16 | DYDC2 |
| RSPH4A | 0.75 | 1.52731754 | 0.5 | 0.502 | 0.003 | 16 | RSPH4A |
| SLC44A4 | 0.75 | 1.369600805 | 0.5 | 0.558 | 0.069 | 16 | SLC44A4 |
| UFC1 | 0.75 | 1.207493768 | 0.5 | 0.62 | 0.146 | 16 | UFC1 |
| DNALI1 | 0.748 | 1.503484069 | 0.496 | 0.502 | 0.008 | 16 | DNALI1 |
| CKB | 0.748 | 1.405869453 | 0.496 | 0.558 | 0.073 | 16 | CKB |
| NME5 | 0.747 | 1.46519066 | 0.494 | 0.504 | 0.012 | 16 | NME5 |
| TEKT1 | 0.746 | 1.426398375 | 0.492 | 0.494 | 0.001 | 16 | TEKT1 |
| ODF3B | 0.745 | 1.486202277 | 0.49 | 0.524 | 0.041 | 16 | ODF3B |
| LGALS35 | 0.744 | 0.72822253 | 0.488 | 0.908 | 0.606 | 16 | LGALS3 |
| C9orf135 | 0.743 | 1.475758578 | 0.486 | 0.49 | 0.005 | 16 | C9orf135 |
| ALDH1A17 | 0.743 | 0.886580475 | 0.486 | 0.779 | 0.353 | 16 | ALDH1A1 |
| WDR78 | 0.742 | 1.450450463 | 0.484 | 0.492 | 0.009 | 16 | WDR78 |
| ODF2L | 0.741 | 1.271449989 | 0.482 | 0.56 | 0.085 | 16 | ODF2L |
| HSPH1 | 0.74 | 1.192036692 | 0.48 | 0.6 | 0.135 | 16 | HSPH1 |
| ALDH3B1 | 0.738 | 1.391939041 | 0.476 | 0.5 | 0.028 | 16 | ALDH3B1 |
| TSPAN6 | 0.737 | 1.25257275 | 0.474 | 0.556 | 0.102 | 16 | TSPAN6 |
| LRRC23 | 0.735 | 1.336629394 | 0.47 | 0.482 | 0.013 | 16 | LRRC23 |
| WDR52 | 0.734 | 1.442612341 | 0.468 | 0.49 | 0.026 | 16 | WDR52 |
| CTSS1 | 0.733 | 0.876911235 | 0.466 | 0.661 | 0.228 | 16 | CTSS |
| MS4A8 | 0.732 | 1.469566634 | 0.464 | 0.468 | 0.004 | 16 | MS4A8 |
| SPAG16 | 0.732 | 1.287236157 | 0.464 | 0.534 | 0.083 | 16 | SPAG16 |
| ENKUR | 0.73 | 1.497377545 | 0.46 | 0.462 | 0.002 | 16 | ENKUR |
| HMGN31 | 0.729 | 1.038376238 | 0.458 | 0.655 | 0.24 | 16 | HMGN3 |
| EFHC1 | 0.728 | 1.378206582 | 0.456 | 0.472 | 0.016 | 16 | EFHC1 |
| PSCA1 | 0.728 | 1.230285256 | 0.456 | 0.55 | 0.096 | 16 | PSCA |
| NUDC | 0.728 | 1.095229168 | 0.456 | 0.554 | 0.112 | 16 | NUDC |
| KIF21A | 0.726 | 1.130406442 | 0.452 | 0.57 | 0.131 | 16 | KIF21A |
| ZBBX | 0.725 | 1.378917292 | 0.45 | 0.452 | 0.001 | 16 | ZBBX |
| MLF1 | 0.725 | 1.285518462 | 0.45 | 0.488 | 0.041 | 16 | MLF1 |
| RSPH9 | 0.724 | 1.511513455 | 0.448 | 0.452 | 0.005 | 16 | RSPH9 |
| C1orf192 | 0.724 | 1.365765905 | 0.448 | 0.45 | 0.002 | 16 | C1orf192 |
| CCDC11 | 0.723 | 1.396902606 | 0.446 | 0.45 | 0.004 | 16 | CCDC11 |
| CCDC113 | 0.723 | 1.353820998 | 0.446 | 0.454 | 0.009 | 16 | CCDC113 |
| AK7 | 0.721 | 1.352426564 | 0.442 | 0.448 | 0.006 | 16 | AK7 |
| AKAP9 | 0.721 | 0.89301493 | 0.442 | 0.709 | 0.311 | 16 | AKAP9 |
| LDLRAD1 | 0.72 | 1.427703183 | 0.44 | 0.444 | 0.004 | 16 | LDLRAD1 |
| KIF9 | 0.72 | 1.29604695 | 0.44 | 0.46 | 0.022 | 16 | KIF9 |
| EFCAB10 | 0.72 | 1.291414369 | 0.44 | 0.442 | 0.002 | 16 | EFCAB10 |
| WDR96 | 0.719 | 1.405127027 | 0.438 | 0.44 | 0.002 | 16 | WDR96 |
| WDR54 | 0.719 | 1.343764025 | 0.438 | 0.458 | 0.02 | 16 | WDR54 |
| C12orf75 | 0.719 | 1.164278553 | 0.438 | 0.49 | 0.051 | 16 | C12orf75 |
| HSPB111 | 0.719 | 1.099523648 | 0.438 | 0.554 | 0.13 | 16 | HSPB11 |
| DYNLRB2 | 0.718 | 1.326289323 | 0.436 | 0.44 | 0.004 | 16 | DYNLRB2 |
| FXYD310 | 0.717 | 0.646149915 | 0.434 | 0.859 | 0.519 | 16 | FXYD3 |
| HSBP1 | 0.716 | 0.828222328 | 0.432 | 0.677 | 0.293 | 16 | HSBP1 |
| TSTD1 | 0.715 | 0.969339665 | 0.43 | 0.612 | 0.217 | 16 | TSTD1 |
| AKAP14 | 0.714 | 1.380683824 | 0.428 | 0.43 | 0.002 | 16 | AKAP14 |
| C11orf70 | 0.714 | 1.32686736 | 0.428 | 0.436 | 0.009 | 16 | C11orf70 |
| WDR86-AS1 | 0.713 | 1.389853372 | 0.426 | 0.438 | 0.011 | 16 | WDR86-AS1 |
| C10orf107 | 0.713 | 1.368555729 | 0.426 | 0.428 | 0.003 | 16 | C10orf107 |
| CES1 | 0.713 | 1.314194208 | 0.426 | 0.466 | 0.046 | 16 | CES1 |
| MNS1 | 0.713 | 1.304457063 | 0.426 | 0.432 | 0.006 | 16 | MNS1 |
| SPEF2 | 0.713 | 1.280941583 | 0.426 | 0.442 | 0.017 | 16 | SPEF2 |
| SPATA18 | 0.713 | 1.258379061 | 0.426 | 0.44 | 0.016 | 16 | SPATA18 |
| CCDC17 | 0.712 | 1.393627067 | 0.424 | 0.428 | 0.004 | 16 | CCDC17 |
| NPHP1 | 0.712 | 1.297426139 | 0.424 | 0.436 | 0.013 | 16 | NPHP1 |
| DPY30 | 0.712 | 1.01622312 | 0.424 | 0.554 | 0.146 | 16 | DPY30 |
| TAX1BP1 | 0.712 | 0.828779467 | 0.424 | 0.691 | 0.314 | 16 | TAX1BP1 |
| TCTEX1D2 | 0.711 | 1.308588452 | 0.422 | 0.444 | 0.025 | 16 | TCTEX1D2 |
| ARHGAP18 | 0.711 | 1.117027661 | 0.422 | 0.524 | 0.113 | 16 | ARHGAP18 |
| PPIL6 | 0.71 | 1.28904542 | 0.42 | 0.426 | 0.006 | 16 | PPIL6 |
| C14orf142 | 0.71 | 1.205251057 | 0.42 | 0.456 | 0.039 | 16 | C14orf142 |
| C21orf59 | 0.71 | 1.109921558 | 0.42 | 0.494 | 0.081 | 16 | C21orf59 |
| GSTA2 | 0.709 | 1.901601974 | 0.418 | 0.422 | 0.005 | 16 | GSTA2 |
| CCDC19 | 0.709 | 1.279306399 | 0.418 | 0.42 | 0.002 | 16 | CCDC19 |
| TMEM231 | 0.709 | 1.227508653 | 0.418 | 0.428 | 0.01 | 16 | TMEM231 |
| C6orf118 | 0.708 | 1.310482374 | 0.416 | 0.418 | 0.001 | 16 | C6orf118 |
| STOML3 | 0.708 | 1.261535694 | 0.416 | 0.418 | 0.001 | 16 | STOML3 |
| FANK1 | 0.708 | 1.180461575 | 0.416 | 0.422 | 0.005 | 16 | FANK1 |
| SEPW1 | 0.708 | 0.884804209 | 0.416 | 0.647 | 0.277 | 16 | SEPW1 |
| ALCAM1 | 0.708 | 0.77838949 | 0.416 | 0.673 | 0.271 | 16 | ALCAM |
| ANXA27 | 0.708 | 0.546523729 | 0.416 | 0.9 | 0.584 | 16 | ANXA2 |
| SPAG1 | 0.707 | 1.238827244 | 0.414 | 0.434 | 0.021 | 16 | SPAG1 |
| CSPP1 | 0.706 | 1.21286586 | 0.412 | 0.454 | 0.044 | 16 | CSPP1 |
| DHRS91 | 0.706 | 1.203572942 | 0.412 | 0.462 | 0.056 | 16 | DHRS9 |
| MRPS31 | 0.705 | 1.16507543 | 0.41 | 0.498 | 0.1 | 16 | MRPS31 |
| TSPAN31 | 0.703 | 0.805151423 | 0.406 | 0.641 | 0.262 | 16 | TSPAN3 |
| CYSTM1 | 0.702 | 0.938814152 | 0.404 | 0.536 | 0.144 | 16 | CYSTM1 |
| RP11-867G2.2 | 0.701 | 1.293094322 | 0.402 | 0.404 | 0.001 | 16 | RP11-867G2.2 |
| SRI | 0.7 | 0.903559314 | 0.4 | 0.596 | 0.226 | 16 | SRI |
| NEK10 | 0.699 | 1.333457937 | 0.398 | 0.4 | 0.002 | 16 | NEK10 |
| ANKUB1 | 0.699 | 1.283968337 | 0.398 | 0.402 | 0.003 | 16 | ANKUB1 |
| DPCD | 0.699 | 1.099863006 | 0.398 | 0.428 | 0.032 | 16 | DPCD |
| CATSPERD | 0.698 | 1.336678292 | 0.396 | 0.398 | 0.001 | 16 | CATSPERD |
| CCDC39 | 0.698 | 1.270844317 | 0.396 | 0.402 | 0.005 | 16 | CCDC39 |
| NWD1 | 0.698 | 1.145924194 | 0.396 | 0.43 | 0.035 | 16 | NWD1 |
| SYNE1 | 0.698 | 1.128809234 | 0.396 | 0.448 | 0.048 | 16 | SYNE1 |
| MORN5 | 0.697 | 1.322881691 | 0.394 | 0.396 | 0.002 | 16 | MORN5 |
| CD164 | 0.696 | 0.824140869 | 0.392 | 0.643 | 0.293 | 16 | CD164 |
| S100A69 | 0.695 | 0.492196777 | 0.39 | 0.958 | 0.816 | 16 | S100A6 |
| CLDN71 | 0.694 | 0.845831221 | 0.388 | 0.552 | 0.175 | 16 | CLDN7 |
| SAMHD11 | 0.692 | 0.853993228 | 0.384 | 0.488 | 0.101 | 16 | SAMHD1 |
| DNPH1 | 0.691 | 0.932800775 | 0.382 | 0.51 | 0.139 | 16 | DNPH1 |
| SPAG17 | 0.69 | 1.286223666 | 0.38 | 0.382 | 0.002 | 16 | SPAG17 |
| RP11-275I14.4 | 0.69 | 1.223524045 | 0.38 | 0.384 | 0.005 | 16 | RP11-275I14.4 |
| B9D1 | 0.69 | 1.189242811 | 0.38 | 0.408 | 0.03 | 16 | B9D1 |
| WDR66 | 0.69 | 1.171791585 | 0.38 | 0.39 | 0.009 | 16 | WDR66 |
| LRRC46 | 0.69 | 1.15672525 | 0.38 | 0.382 | 0.002 | 16 | LRRC46 |
| MAP3K19 | 0.689 | 1.197082606 | 0.378 | 0.38 | 0.001 | 16 | MAP3K19 |
| LRRC48 | 0.689 | 1.174010883 | 0.378 | 0.384 | 0.005 | 16 | LRRC48 |
| AGR211 | 0.689 | 0.393138381 | 0.378 | 0.785 | 0.43 | 16 | AGR2 |
| EFCAB2 | 0.688 | 1.113579768 | 0.376 | 0.4 | 0.023 | 16 | EFCAB2 |
| LINC00948 | 0.687 | 1.293041703 | 0.374 | 0.376 | 0.002 | 16 | LINC00948 |
| DNAAF1 | 0.687 | 1.229780805 | 0.374 | 0.404 | 0.029 | 16 | DNAAF1 |
| DNAJA4 | 0.686 | 1.171361997 | 0.372 | 0.394 | 0.023 | 16 | DNAJA4 |
| PROM1 | 0.686 | 1.127220829 | 0.372 | 0.422 | 0.055 | 16 | PROM1 |
| CDS1 | 0.686 | 1.10648295 | 0.372 | 0.402 | 0.032 | 16 | CDS1 |
| C9orf117 | 0.685 | 1.315334073 | 0.37 | 0.373 | 0.003 | 16 | C9orf117 |
| FHAD1 | 0.685 | 1.168792582 | 0.37 | 0.378 | 0.008 | 16 | FHAD1 |
| DNAH3 | 0.684 | 1.166042132 | 0.368 | 0.369 | 0.002 | 16 | DNAH3 |
| OSCP1 | 0.684 | 1.114097624 | 0.368 | 0.378 | 0.011 | 16 | OSCP1 |
| FAM174A | 0.684 | 1.051990711 | 0.368 | 0.43 | 0.065 | 16 | FAM174A |
| H2AFJ | 0.684 | 0.805142335 | 0.368 | 0.544 | 0.188 | 16 | H2AFJ |
| IFI272 | 0.684 | 0.69851304 | 0.368 | 0.637 | 0.279 | 16 | IFI27 |
| PERP12 | 0.684 | 0.525094196 | 0.368 | 0.833 | 0.492 | 16 | PERP |
| PIH1D2 | 0.683 | 1.166178868 | 0.366 | 0.371 | 0.005 | 16 | PIH1D2 |
| RABL5 | 0.683 | 1.050216066 | 0.366 | 0.396 | 0.032 | 16 | RABL5 |
| WFDC210 | 0.682 | 0.393373212 | 0.364 | 0.809 | 0.449 | 16 | WFDC2 |
| CCDC173 | 0.681 | 1.216883693 | 0.362 | 0.365 | 0.004 | 16 | CCDC173 |
| IGFBP2 | 0.681 | 0.854171559 | 0.362 | 0.452 | 0.087 | 16 | IGFBP2 |
| SAT18 | 0.681 | 0.446377826 | 0.362 | 0.89 | 0.641 | 16 | SAT1 |
| DTHD1 | 0.68 | 1.218976142 | 0.36 | 0.363 | 0.002 | 16 | DTHD1 |
| CCDC42B | 0.68 | 1.151052766 | 0.36 | 0.361 | 0.002 | 16 | CCDC42B |
| DNAH9 | 0.68 | 1.115337676 | 0.36 | 0.363 | 0.002 | 16 | DNAH9 |
| CCDC176 | 0.68 | 1.061587176 | 0.36 | 0.373 | 0.013 | 16 | CCDC176 |
| SOD1 | 0.68 | 0.597329588 | 0.36 | 0.711 | 0.388 | 16 | SOD1 |
| CLU4 | 0.68 | 0.528368799 | 0.36 | 0.667 | 0.321 | 16 | CLU |
| LZTFL1 | 0.679 | 1.015355469 | 0.358 | 0.398 | 0.041 | 16 | LZTFL1 |
| CTGF1 | 0.678 | 0.79308507 | 0.356 | 0.478 | 0.116 | 16 | CTGF |
| CCDC65 | 0.677 | 1.105224202 | 0.354 | 0.355 | 0.002 | 16 | CCDC65 |
| C11orf74 | 0.677 | 1.068872714 | 0.354 | 0.384 | 0.03 | 16 | C11orf74 |
| DSTN2 | 0.677 | 0.583917961 | 0.354 | 0.711 | 0.435 | 16 | DSTN |
| DRC1 | 0.676 | 1.148001154 | 0.352 | 0.353 | 0.001 | 16 | DRC1 |
| CASC1 | 0.676 | 1.065971578 | 0.352 | 0.355 | 0.004 | 16 | CASC1 |
| TRAF3IP1 | 0.676 | 1.018157467 | 0.352 | 0.384 | 0.032 | 16 | TRAF3IP1 |
| COX6A1 | 0.675 | 0.54382558 | 0.35 | 0.779 | 0.483 | 16 | COX6A1 |
| IFT172 | 0.674 | 0.963215858 | 0.348 | 0.394 | 0.048 | 16 | IFT172 |
| CCDC104 | 0.674 | 0.930963585 | 0.348 | 0.422 | 0.078 | 16 | CCDC104 |
| YWHAE | 0.674 | 0.584290625 | 0.348 | 0.671 | 0.364 | 16 | YWHAE |
| TMBIM6 | 0.674 | 0.520334167 | 0.348 | 0.797 | 0.541 | 16 | TMBIM6 |
| SLC27A2 | 0.673 | 0.887034038 | 0.346 | 0.422 | 0.074 | 16 | SLC27A2 |
| S100A115 | 0.673 | 0.506797293 | 0.346 | 0.807 | 0.549 | 16 | S100A11 |
| LRP11 | 0.671 | 0.948523166 | 0.342 | 0.39 | 0.049 | 16 | LRP11 |
| TXN3 | 0.67 | 0.503287014 | 0.34 | 0.785 | 0.496 | 16 | TXN |
| ALOX157 | 0.67 | 0.302823932 | 0.34 | 0.781 | 0.388 | 16 | ALOX15 |
| IFT43 | 0.669 | 0.931156239 | 0.338 | 0.394 | 0.057 | 16 | IFT43 |
| STK33 | 0.668 | 1.112217354 | 0.336 | 0.343 | 0.009 | 16 | STK33 |
| ARMC4 | 0.668 | 1.08109147 | 0.336 | 0.337 | 0.001 | 16 | ARMC4 |
| DZIP3 | 0.668 | 0.985670184 | 0.336 | 0.365 | 0.029 | 16 | DZIP3 |
| RAB11FIP11 | 0.668 | 0.673576707 | 0.336 | 0.5 | 0.161 | 16 | RAB11FIP1 |
| UBXN10 | 0.667 | 1.024223526 | 0.334 | 0.335 | 0.002 | 16 | UBXN10 |
| IFT81 | 0.667 | 0.881303452 | 0.334 | 0.388 | 0.052 | 16 | IFT81 |
| IGFBP73 | 0.667 | 0.452048077 | 0.334 | 0.633 | 0.291 | 16 | IGFBP7 |
| TMEM591 | 0.666 | 0.521892489 | 0.332 | 0.773 | 0.518 | 16 | TMEM59 |
| ELF310 | 0.666 | 0.442280815 | 0.332 | 0.739 | 0.395 | 16 | ELF3 |
| TTC18 | 0.665 | 1.158278206 | 0.33 | 0.341 | 0.012 | 16 | TTC18 |
| CYB5A1 | 0.665 | 0.70686151 | 0.33 | 0.524 | 0.204 | 16 | CYB5A |
| CAST1 | 0.665 | 0.59053961 | 0.33 | 0.647 | 0.335 | 16 | CAST |
| UBB1 | 0.665 | 0.426018095 | 0.33 | 0.882 | 0.748 | 16 | UBB |
| DNAH11 | 0.664 | 1.153228237 | 0.328 | 0.333 | 0.005 | 16 | DNAH11 |
| C7orf57 | 0.664 | 1.020858097 | 0.328 | 0.329 | 0.001 | 16 | C7orf57 |
| MUC161 | 0.664 | 0.917520291 | 0.328 | 0.392 | 0.064 | 16 | MUC16 |
| PTGES3 | 0.664 | 0.664609015 | 0.328 | 0.53 | 0.213 | 16 | PTGES3 |
| TTC29 | 0.663 | 1.096686339 | 0.326 | 0.327 | 0.001 | 16 | TTC29 |
| PPP1R42 | 0.663 | 1.066337651 | 0.326 | 0.327 | 0.002 | 16 | PPP1R42 |
| CLDN3 | 0.663 | 1.006834884 | 0.326 | 0.398 | 0.078 | 16 | CLDN3 |
| TUSC3 | 0.662 | 0.915137373 | 0.324 | 0.376 | 0.052 | 16 | TUSC3 |
| TCTN1 | 0.662 | 0.904310234 | 0.324 | 0.351 | 0.027 | 16 | TCTN1 |
| POLR2I | 0.662 | 0.787450089 | 0.324 | 0.44 | 0.119 | 16 | POLR2I |
| CCDC78 | 0.66 | 0.992119875 | 0.32 | 0.321 | 0.002 | 16 | CCDC78 |
| RUVBL2 | 0.66 | 0.968800853 | 0.32 | 0.373 | 0.057 | 16 | RUVBL2 |
| TNFAIP8L1 | 0.659 | 1.015595206 | 0.318 | 0.329 | 0.011 | 16 | TNFAIP8L1 |
| CC2D2A | 0.659 | 0.980564707 | 0.318 | 0.329 | 0.012 | 16 | CC2D2A |
| GDF151 | 0.659 | 0.791074831 | 0.318 | 0.424 | 0.101 | 16 | GDF15 |
| CDHR4 | 0.658 | 1.055255046 | 0.316 | 0.317 | 0.001 | 16 | CDHR4 |
| DNAL1 | 0.658 | 0.963923627 | 0.316 | 0.337 | 0.021 | 16 | DNAL1 |
| TAGLN2 | 0.658 | 0.560009678 | 0.316 | 0.643 | 0.345 | 16 | TAGLN2 |
| ECT2L | 0.657 | 1.047207734 | 0.314 | 0.315 | 0.002 | 16 | ECT2L |
| RUVBL1 | 0.657 | 0.855527034 | 0.314 | 0.353 | 0.039 | 16 | RUVBL1 |
| SYAP1 | 0.657 | 0.700794102 | 0.314 | 0.502 | 0.194 | 16 | SYAP1 |
| METTL7A | 0.657 | 0.641010989 | 0.314 | 0.55 | 0.249 | 16 | METTL7A |
| DNAH7 | 0.655 | 1.12151599 | 0.31 | 0.315 | 0.006 | 16 | DNAH7 |
| IQCD | 0.655 | 0.930529806 | 0.31 | 0.313 | 0.003 | 16 | IQCD |
| NDUFB1 | 0.655 | 0.551762364 | 0.31 | 0.649 | 0.377 | 16 | NDUFB1 |
| UBL5 | 0.655 | 0.52627869 | 0.31 | 0.715 | 0.472 | 16 | UBL5 |
| RP4-666F24.3 | 0.654 | 1.016516177 | 0.308 | 0.309 | 0.001 | 16 | RP4-666F24.3 |
| C9orf9 | 0.654 | 0.922003618 | 0.308 | 0.315 | 0.007 | 16 | C9orf9 |
| C21orf58 | 0.653 | 1.05013662 | 0.306 | 0.313 | 0.008 | 16 | C21orf58 |
| ANKRD66 | 0.653 | 1.048598358 | 0.306 | 0.307 | 0 | 16 | ANKRD66 |
| EPCAM | 0.653 | 0.676798859 | 0.306 | 0.448 | 0.142 | 16 | EPCAM |
| PCDP1 | 0.652 | 0.984270112 | 0.304 | 0.305 | 0.002 | 16 | PCDP1 |
| CMPK1 | 0.651 | 0.650482585 | 0.302 | 0.508 | 0.222 | 16 | CMPK1 |
| TMEM14B | 0.651 | 0.636920585 | 0.302 | 0.466 | 0.165 | 16 | TMEM14B |
| MORF4L2 | 0.651 | 0.575254768 | 0.302 | 0.5 | 0.197 | 16 | MORF4L2 |
| IGHG43 | 0.956 | 3.311435984 | 0.912 | 0.974 | 0.227 | 17 | IGHG4 |
| IGKV3-201 | 0.946 | 3.509862656 | 0.892 | 0.91 | 0.038 | 17 | IGKV3-20 |
| SSR45 | 0.941 | 1.695218843 | 0.882 | 0.996 | 0.568 | 17 | SSR4 |
| IGHG13 | 0.935 | 2.866528992 | 0.87 | 0.949 | 0.195 | 17 | IGHG1 |
| IGHG23 | 0.933 | 2.59483468 | 0.866 | 0.919 | 0.108 | 17 | IGHG2 |
| IGJ8 | 0.915 | 1.708165762 | 0.83 | 0.994 | 0.481 | 17 | IGJ |
| IGHG33 | 0.904 | 2.553627841 | 0.808 | 0.914 | 0.211 | 17 | IGHG3 |
| IGKC1 | 0.894 | 1.228316744 | 0.788 | 0.919 | 0.21 | 17 | IGKC |
| MZB12 | 0.892 | 1.766046639 | 0.784 | 0.908 | 0.153 | 17 | MZB1 |
| AC096579.72 | 0.881 | 2.406300445 | 0.762 | 0.831 | 0.082 | 17 | AC096579.7 |
| SEC11C2 | 0.86 | 1.438339615 | 0.72 | 0.886 | 0.321 | 17 | SEC11C |
| HSP90B12 | 0.857 | 1.311909984 | 0.714 | 0.941 | 0.579 | 17 | HSP90B1 |
| PRDX42 | 0.856 | 1.464976484 | 0.712 | 0.862 | 0.274 | 17 | PRDX4 |
| SPCS32 | 0.821 | 1.282912422 | 0.642 | 0.847 | 0.345 | 17 | SPCS3 |
| IGKV3D-20 | 0.808 | 2.531102793 | 0.616 | 0.631 | 0.016 | 17 | IGKV3D-20 |
| CCND2 | 0.8 | 1.739136989 | 0.6 | 0.682 | 0.103 | 17 | CCND2 |
| FKBP112 | 0.792 | 1.417244944 | 0.584 | 0.695 | 0.134 | 17 | FKBP11 |
| HERPUD12 | 0.792 | 1.103999917 | 0.584 | 0.809 | 0.33 | 17 | HERPUD1 |
| FAM46C2 | 0.781 | 1.310311028 | 0.562 | 0.686 | 0.146 | 17 | FAM46C |
| RNA28S51 | 0.775 | 0.691893923 | 0.55 | 0.961 | 0.79 | 17 | RNA28S5 |
| ENAM2 | 0.772 | 1.305438033 | 0.544 | 0.67 | 0.114 | 17 | ENAM |
| TSC22D33 | 0.771 | 1.078024443 | 0.542 | 0.825 | 0.4 | 17 | TSC22D3 |
| DERL32 | 0.765 | 1.266776978 | 0.53 | 0.635 | 0.102 | 17 | DERL3 |
| IGHA17 | 0.765 | 0.752446432 | 0.53 | 0.941 | 0.551 | 17 | IGHA1 |
| SSR32 | 0.751 | 1.012181339 | 0.502 | 0.725 | 0.301 | 17 | SSR3 |
| IGHV1-18 | 0.747 | 3.686718649 | 0.494 | 0.515 | 0.029 | 17 | IGHV1-18 |
| IGKV3-11 | 0.747 | 2.305422865 | 0.494 | 0.517 | 0.021 | 17 | IGKV3-11 |
| PIM22 | 0.747 | 1.371560581 | 0.494 | 0.58 | 0.09 | 17 | PIM2 |
| RRBP12 | 0.744 | 1.012586865 | 0.488 | 0.721 | 0.304 | 17 | RRBP1 |
| SLAMF72 | 0.743 | 1.244781128 | 0.486 | 0.562 | 0.075 | 17 | SLAMF7 |
| FCRL52 | 0.734 | 1.334571935 | 0.468 | 0.517 | 0.051 | 17 | FCRL5 |
| XBP110 | 0.727 | 0.762242797 | 0.454 | 0.829 | 0.453 | 17 | XBP1 |
| ITM2C2 | 0.726 | 1.076298725 | 0.452 | 0.591 | 0.158 | 17 | ITM2C |
| SUB12 | 0.725 | 0.911675318 | 0.45 | 0.709 | 0.334 | 17 | SUB1 |
| IGKV3OR2-268 | 0.72 | 2.04389055 | 0.44 | 0.448 | 0.008 | 17 | IGKV3OR2-268 |
| FKBP22 | 0.72 | 0.936224838 | 0.44 | 0.635 | 0.236 | 17 | FKBP2 |
| TRAM12 | 0.72 | 0.865567154 | 0.44 | 0.707 | 0.344 | 17 | TRAM1 |
| PDIA61 | 0.715 | 0.859093121 | 0.43 | 0.721 | 0.388 | 17 | PDIA6 |
| IGHA23 | 0.711 | 1.236035839 | 0.422 | 0.664 | 0.237 | 17 | IGHA2 |
| ERLEC12 | 0.707 | 0.893495236 | 0.414 | 0.629 | 0.251 | 17 | ERLEC1 |
| SEL1L2 | 0.7 | 0.955633968 | 0.4 | 0.546 | 0.161 | 17 | SEL1L |
| SPCS22 | 0.7 | 0.948011707 | 0.4 | 0.558 | 0.184 | 17 | SPCS2 |
| CD79A2 | 0.698 | 1.077604505 | 0.396 | 0.462 | 0.064 | 17 | CD79A |
| IGHV4-39 | 0.697 | 2.978773375 | 0.394 | 0.42 | 0.031 | 17 | IGHV4-39 |
| FTL4 | 0.692 | 0.359043274 | 0.384 | 0.959 | 0.823 | 17 | FTL |
| SPCS12 | 0.69 | 0.742633175 | 0.38 | 0.668 | 0.346 | 17 | SPCS1 |
| PRDM12 | 0.689 | 1.139843646 | 0.378 | 0.458 | 0.083 | 17 | PRDM1 |
| RGS14 | 0.687 | 0.74856402 | 0.374 | 0.578 | 0.193 | 17 | RGS1 |
| C19orf102 | 0.686 | 0.8502445 | 0.372 | 0.587 | 0.253 | 17 | C19orf10 |
| UBE2J12 | 0.685 | 0.924706476 | 0.37 | 0.513 | 0.16 | 17 | UBE2J1 |
| IGHV1-24 | 0.684 | 3.575662036 | 0.368 | 0.415 | 0.051 | 17 | IGHV1-24 |
| LY96 | 0.681 | 1.024204619 | 0.362 | 0.442 | 0.083 | 17 | LY96 |
| MTDH | 0.681 | 0.837928046 | 0.362 | 0.591 | 0.269 | 17 | MTDH |
| IGKV4-11 | 0.679 | 1.202499425 | 0.358 | 0.407 | 0.049 | 17 | IGKV4-1 |
| IGKJ5 | 0.676 | 2.64331821 | 0.352 | 0.367 | 0.018 | 17 | IGKJ5 |
| ISG202 | 0.676 | 0.884813294 | 0.352 | 0.475 | 0.127 | 17 | ISG20 |
| PPIB2 | 0.676 | 0.699507117 | 0.352 | 0.69 | 0.389 | 17 | PPIB |
| IL5RA | 0.674 | 1.215110724 | 0.348 | 0.367 | 0.021 | 17 | IL5RA |
| CRELD21 | 0.674 | 0.940680746 | 0.348 | 0.458 | 0.121 | 17 | CRELD2 |
| PDIA41 | 0.674 | 0.826976681 | 0.348 | 0.53 | 0.202 | 17 | PDIA4 |
| SELK2 | 0.672 | 0.607583699 | 0.344 | 0.656 | 0.348 | 17 | SELK |
| IGKV3D-11 | 0.671 | 1.371605866 | 0.342 | 0.352 | 0.009 | 17 | IGKV3D-11 |
| RPN21 | 0.669 | 0.748204269 | 0.338 | 0.587 | 0.289 | 17 | RPN2 |
| CYBA2 | 0.669 | 0.714489621 | 0.338 | 0.519 | 0.193 | 17 | CYBA |
| SEC61B1 | 0.669 | 0.626403165 | 0.338 | 0.65 | 0.356 | 17 | SEC61B |
| PPAPDC1B1 | 0.668 | 0.865449175 | 0.336 | 0.47 | 0.15 | 17 | PPAPDC1B |
| IGHV1-8 | 0.665 | 2.670502429 | 0.33 | 0.342 | 0.013 | 17 | IGHV1-8 |
| IGKV3-7 | 0.665 | 1.606178303 | 0.33 | 0.342 | 0.011 | 17 | IGKV3-7 |
| SDF2L12 | 0.665 | 0.907586026 | 0.33 | 0.43 | 0.105 | 17 | SDF2L1 |
| HLA-C1 | 0.665 | 0.431809987 | 0.33 | 0.894 | 0.663 | 17 | HLA-C |
| HSPA51 | 0.664 | 0.559463284 | 0.328 | 0.739 | 0.472 | 17 | HSPA5 |
| LMAN1 | 0.66 | 0.710336888 | 0.32 | 0.525 | 0.228 | 17 | LMAN1 |
| VIMP2 | 0.652 | 0.655564766 | 0.304 | 0.558 | 0.281 | 17 | VIMP |
| TPSAB1 | 0.998 | 5.174439086 | 0.996 | 0.996 | 0.026 | 18 | TPSAB1 |
| CPA3 | 0.986 | 4.52857375 | 0.972 | 0.974 | 0.016 | 18 | CPA3 |
| CD691 | 0.953 | 3.427734839 | 0.906 | 0.927 | 0.08 | 18 | CD69 |
| SRGN4 | 0.944 | 2.243966539 | 0.888 | 0.974 | 0.313 | 18 | SRGN |
| HPGD | 0.901 | 3.415146612 | 0.802 | 0.846 | 0.16 | 18 | HPGD |
| RGS15 | 0.86 | 2.019609194 | 0.72 | 0.835 | 0.194 | 18 | RGS1 |
| HPGDS | 0.843 | 2.943679477 | 0.686 | 0.689 | 0.007 | 18 | HPGDS |
| SLC18A2 | 0.833 | 2.785736346 | 0.666 | 0.674 | 0.014 | 18 | SLC18A2 |
| SAMSN11 | 0.821 | 2.248770059 | 0.642 | 0.685 | 0.069 | 18 | SAMSN1 |
| KIT | 0.809 | 2.657150947 | 0.618 | 0.63 | 0.025 | 18 | KIT |
| NFKBIZ | 0.806 | 1.779600078 | 0.612 | 0.74 | 0.274 | 18 | NFKBIZ |
| HDC | 0.801 | 2.726082007 | 0.602 | 0.604 | 0.006 | 18 | HDC |
| CTSG | 0.795 | 3.809576386 | 0.59 | 0.593 | 0.007 | 18 | CTSG |
| ACSL4 | 0.793 | 2.24782145 | 0.586 | 0.641 | 0.112 | 18 | ACSL4 |
| FTH14 | 0.788 | 0.991705563 | 0.576 | 0.908 | 0.727 | 18 | FTH1 |
| LAPTM51 | 0.783 | 2.004315262 | 0.566 | 0.623 | 0.077 | 18 | LAPTM5 |
| TMSB4X10 | 0.779 | 0.794678887 | 0.558 | 0.941 | 0.692 | 18 | TMSB4X |
| TNFAIP32 | 0.777 | 1.860708062 | 0.554 | 0.652 | 0.158 | 18 | TNFAIP3 |
| TPSD1 | 0.774 | 2.776116909 | 0.548 | 0.549 | 0.002 | 18 | TPSD1 |
| PTGS2 | 0.767 | 2.691449 | 0.534 | 0.571 | 0.07 | 18 | PTGS2 |
| GATA21 | 0.766 | 2.246371859 | 0.532 | 0.557 | 0.036 | 18 | GATA2 |
| NFKBIA1 | 0.766 | 1.171279287 | 0.532 | 0.81 | 0.506 | 18 | NFKBIA |
| PPP1R15A2 | 0.766 | 1.102008175 | 0.532 | 0.832 | 0.559 | 18 | PPP1R15A |
| CD522 | 0.765 | 1.712431679 | 0.53 | 0.626 | 0.123 | 18 | CD52 |
| IL1RL1 | 0.76 | 2.237688648 | 0.52 | 0.524 | 0.006 | 18 | IL1RL1 |
| VIM6 | 0.755 | 1.085969223 | 0.51 | 0.773 | 0.348 | 18 | VIM |
| FTL5 | 0.746 | 0.577984644 | 0.492 | 0.938 | 0.825 | 18 | FTL |
| DUSP6 | 0.741 | 1.852536955 | 0.482 | 0.593 | 0.198 | 18 | DUSP6 |
| AHR | 0.74 | 1.601803981 | 0.48 | 0.601 | 0.203 | 18 | AHR |
| MS4A2 | 0.734 | 2.180620924 | 0.468 | 0.469 | 0.002 | 18 | MS4A2 |
| CD632 | 0.732 | 0.921403213 | 0.464 | 0.773 | 0.585 | 18 | CD63 |
| NR4A21 | 0.726 | 1.608884979 | 0.452 | 0.575 | 0.191 | 18 | NR4A2 |
| C1orf186 | 0.723 | 2.286431642 | 0.446 | 0.451 | 0.008 | 18 | C1orf186 |
| VWA5A | 0.722 | 1.982134468 | 0.444 | 0.473 | 0.044 | 18 | VWA5A |
| CLU5 | 0.721 | 1.134434693 | 0.442 | 0.659 | 0.325 | 18 | CLU |
| AREG1 | 0.716 | 2.024546324 | 0.432 | 0.48 | 0.069 | 18 | AREG |
| SELK3 | 0.716 | 1.269659065 | 0.432 | 0.645 | 0.351 | 18 | SELK |
| RGS21 | 0.714 | 1.573533683 | 0.428 | 0.549 | 0.168 | 18 | RGS2 |
| CCL4 | 0.711 | 2.348953506 | 0.422 | 0.476 | 0.076 | 18 | CCL4 |
| ANXA19 | 0.704 | 0.68060801 | 0.408 | 0.864 | 0.69 | 18 | ANXA1 |
| ALOX5AP | 0.696 | 1.771621885 | 0.392 | 0.418 | 0.033 | 18 | ALOX5AP |
| GLUL6 | 0.695 | 1.048698358 | 0.39 | 0.681 | 0.46 | 18 | GLUL |
| TYROBP1 | 0.694 | 1.409613128 | 0.388 | 0.44 | 0.052 | 18 | TYROBP |
| GPR65 | 0.693 | 2.000234525 | 0.386 | 0.41 | 0.029 | 18 | GPR65 |
| RGS131 | 0.692 | 2.156904871 | 0.384 | 0.388 | 0.005 | 18 | RGS13 |
| S100A44 | 0.691 | 0.98205822 | 0.382 | 0.645 | 0.356 | 18 | S100A4 |
| FOSB3 | 0.689 | 0.780619806 | 0.378 | 0.766 | 0.63 | 18 | FOSB |
| CAPG | 0.682 | 1.567780116 | 0.364 | 0.425 | 0.088 | 18 | CAPG |
| UBB2 | 0.682 | 0.698294255 | 0.364 | 0.821 | 0.75 | 18 | UBB |
| TSC22D34 | 0.673 | 0.961192906 | 0.346 | 0.63 | 0.408 | 18 | TSC22D3 |
| FCER1G1 | 0.669 | 1.476989129 | 0.338 | 0.374 | 0.04 | 18 | FCER1G |
| GCSAML | 0.668 | 1.739271194 | 0.336 | 0.337 | 0.001 | 18 | GCSAML |
| PTMA4 | 0.668 | 0.624269996 | 0.336 | 0.784 | 0.7 | 18 | PTMA |
| ALAS1 | 0.667 | 1.850535587 | 0.334 | 0.396 | 0.091 | 18 | ALAS1 |
| CTSD1 | 0.665 | 1.115424206 | 0.33 | 0.56 | 0.349 | 18 | CTSD |
| NR4A11 | 0.659 | 0.772982596 | 0.318 | 0.648 | 0.482 | 18 | NR4A1 |
| KLF61 | 0.658 | 0.804560648 | 0.316 | 0.703 | 0.546 | 18 | KLF6 |
| RAC2 | 0.656 | 1.61498032 | 0.312 | 0.341 | 0.036 | 18 | RAC2 |
| BTG2 | 0.654 | 0.720955902 | 0.308 | 0.689 | 0.529 | 18 | BTG2 |
| RP11-354E11.2 | 0.651 | 1.695824119 | 0.302 | 0.304 | 0.002 | 18 | RP11-354E11.2 |
| ARHGDIB2 | 0.651 | 1.149409493 | 0.302 | 0.425 | 0.154 | 18 | ARHGDIB |
| CCL51 | 0.757 | 1.259194823 | 0.514 | 0.575 | 0.058 | 19 | CCL5 |
| CXCR41 | 0.699 | 0.786232299 | 0.398 | 0.556 | 0.147 | 19 | CXCR4 |
| TRBC21 | 0.692 | 0.930657093 | 0.384 | 0.44 | 0.049 | 19 | TRBC2 |
| CD523 | 0.69 | 0.549425561 | 0.38 | 0.527 | 0.126 | 19 | CD52 |
| CD21 | 0.653 | 0.79568316 | 0.306 | 0.348 | 0.035 | 19 | CD2 |
| PTPRC2 | 0.653 | 0.575793471 | 0.306 | 0.42 | 0.098 | 19 | PTPRC |
| HLA-DRA2 | 0.816 | 1.423379626 | 0.632 | 0.776 | 0.261 | 20 | HLA-DRA |
| CD745 | 0.805 | 1.470244949 | 0.61 | 0.82 | 0.454 | 20 | CD74 |
| RPS271 | 0.719 | 1.102606072 | 0.438 | 0.765 | 0.573 | 20 | RPS27 |
| TMSB4X11 | 0.712 | 0.627664909 | 0.424 | 0.869 | 0.694 | 20 | TMSB4X |
| HLA-DRB12 | 0.708 | 1.157465287 | 0.416 | 0.563 | 0.205 | 20 | HLA-DRB1 |
| MTRNR2L14 | 0.702 | 0.706864257 | 0.404 | 0.885 | 0.826 | 20 | MTRNR2L1 |
| HLA-DPB11 | 0.692 | 1.198919055 | 0.384 | 0.481 | 0.114 | 20 | HLA-DPB1 |
| CD524 | 0.681 | 1.501954488 | 0.362 | 0.464 | 0.127 | 20 | CD52 |
| SRGN5 | 0.669 | 0.618223683 | 0.338 | 0.612 | 0.319 | 20 | SRGN |
| CXCR42 | 0.665 | 1.327064142 | 0.33 | 0.443 | 0.149 | 20 | CXCR4 |
| MTRNR2L21 | 0.663 | 0.750673345 | 0.326 | 0.781 | 0.682 | 20 | MTRNR2L2 |
| RPL391 | 0.66 | 0.707668969 | 0.32 | 0.743 | 0.674 | 20 | RPL39 |
| HLA-DPA12 | 0.659 | 1.189281081 | 0.318 | 0.432 | 0.143 | 20 | HLA-DPA1 |
| LAPTM52 | 0.655 | 1.30411285 | 0.31 | 0.383 | 0.082 | 20 | LAPTM5 |
| GPR1831 | 0.654 | 1.622192173 | 0.308 | 0.377 | 0.079 | 20 | GPR183 |
| MTRNR2L8 | 0.652 | 0.464223993 | 0.304 | 0.858 | 0.78 | 20 | MTRNR2L8 |
| LTF2 | 0.942 | 2.093654751 | 0.884 | 0.991 | 0.184 | 21 | LTF |
| LYZ6 | 0.934 | 2.068722484 | 0.868 | 0.991 | 0.348 | 21 | LYZ |
| ZG16B2 | 0.929 | 1.922438286 | 0.858 | 0.982 | 0.237 | 21 | ZG16B |
| SLPI13 | 0.924 | 1.582559228 | 0.848 | 1 | 0.747 | 21 | SLPI |
| STATH12 | 0.923 | 1.969498437 | 0.846 | 0.982 | 0.397 | 21 | STATH |
| AZGP12 | 0.915 | 1.726434423 | 0.83 | 0.963 | 0.149 | 21 | AZGP1 |
| PIGR7 | 0.913 | 1.538608104 | 0.826 | 1 | 0.35 | 21 | PIGR |
| TCN12 | 0.912 | 1.668070929 | 0.824 | 0.954 | 0.158 | 21 | TCN1 |
| DMBT12 | 0.911 | 1.983097291 | 0.822 | 0.917 | 0.118 | 21 | DMBT1 |
| BPIFB16 | 0.911 | 1.831454673 | 0.822 | 0.982 | 0.413 | 21 | BPIFB1 |
| C6orf582 | 0.891 | 1.902955612 | 0.782 | 0.89 | 0.125 | 21 | C6orf58 |
| BPIFA16 | 0.886 | 1.914599677 | 0.772 | 0.972 | 0.479 | 21 | BPIFA1 |
| PIP2 | 0.86 | 1.85843242 | 0.72 | 0.835 | 0.126 | 21 | PIP |
| ODAM1 | 0.839 | 1.594091702 | 0.678 | 0.78 | 0.107 | 21 | ODAM |
| RP11-1143G9.42 | 0.836 | 2.054456351 | 0.672 | 0.789 | 0.139 | 21 | RP11-1143G9.4 |
| RNASE11 | 0.799 | 1.302799105 | 0.598 | 0.716 | 0.123 | 21 | RNASE1 |
| CXCL177 | 0.758 | 0.763003361 | 0.516 | 0.844 | 0.323 | 21 | CXCL17 |
| NUCB22 | 0.755 | 0.823560944 | 0.51 | 0.789 | 0.276 | 21 | NUCB2 |
| WFDC213 | 0.746 | 0.672454192 | 0.492 | 0.927 | 0.456 | 21 | WFDC2 |
| NDRG21 | 0.745 | 0.957609195 | 0.49 | 0.651 | 0.17 | 21 | NDRG2 |
| SLC12A21 | 0.737 | 0.829084707 | 0.474 | 0.67 | 0.193 | 21 | SLC12A2 |
| CCL281 | 0.731 | 1.079182067 | 0.462 | 0.541 | 0.079 | 21 | CCL28 |
| XBP112 | 0.725 | 0.634492853 | 0.45 | 0.853 | 0.46 | 21 | XBP1 |
| PHLDA11 | 0.713 | 0.739197833 | 0.426 | 0.587 | 0.149 | 21 | PHLDA1 |
| KIAA13241 | 0.708 | 0.761449485 | 0.416 | 0.541 | 0.115 | 21 | KIAA1324 |
| PPP1R1B1 | 0.7 | 0.880765889 | 0.4 | 0.45 | 0.046 | 21 | PPP1R1B |
| OXR1 | 0.698 | 0.653546674 | 0.396 | 0.55 | 0.14 | 21 | OXR1 |
| LRRC261 | 0.695 | 0.86654118 | 0.39 | 0.459 | 0.067 | 21 | LRRC26 |
| SCGB3A12 | 0.693 | 1.63814158 | 0.386 | 0.486 | 0.109 | 21 | SCGB3A1 |
| CA21 | 0.689 | 0.978718065 | 0.378 | 0.45 | 0.065 | 21 | CA2 |
| MT-ND31 | 0.688 | 0.555046088 | 0.376 | 0.881 | 0.526 | 21 | MT-ND3 |
| FDCSP1 | 0.686 | 1.720570195 | 0.372 | 0.44 | 0.077 | 21 | FDCSP |
| TMED31 | 0.686 | 0.652619617 | 0.372 | 0.569 | 0.192 | 21 | TMED3 |
| EHF2 | 0.685 | 0.499643251 | 0.37 | 0.697 | 0.291 | 21 | EHF |
| CST35 | 0.682 | 0.355096501 | 0.364 | 0.798 | 0.426 | 21 | CST3 |
| MGLL1 | 0.679 | 0.53457469 | 0.358 | 0.532 | 0.159 | 21 | MGLL |
| ADIRF5 | 0.679 | 0.505192693 | 0.358 | 0.725 | 0.345 | 21 | ADIRF |
| PEBP11 | 0.678 | 0.429500193 | 0.356 | 0.798 | 0.441 | 21 | PEBP1 |
| CLDN101 | 0.674 | 0.646020258 | 0.348 | 0.459 | 0.103 | 21 | CLDN10 |
| CLU6 | 0.673 | 0.443953118 | 0.346 | 0.688 | 0.328 | 21 | CLU |
| MT-ND22 | 0.672 | 0.499205883 | 0.344 | 0.872 | 0.586 | 21 | MT-ND2 |
| AQP52 | 0.671 | 0.446382671 | 0.342 | 0.596 | 0.207 | 21 | AQP5 |
| CLDN31 | 0.67 | 0.693450608 | 0.34 | 0.431 | 0.085 | 21 | CLDN3 |
| A2M1 | 0.669 | 0.38035611 | 0.338 | 0.468 | 0.103 | 21 | A2M |
| KRT72 | 0.667 | 0.514277248 | 0.334 | 0.606 | 0.242 | 21 | KRT7 |
| P4HB1 | 0.666 | 0.42556503 | 0.332 | 0.725 | 0.351 | 21 | P4HB |
| FAM3D3 | 0.663 | 0.478247682 | 0.326 | 0.486 | 0.138 | 21 | FAM3D |
| NEAT1 | 0.661 | 0.532012033 | 0.322 | 0.642 | 0.302 | 21 | NEAT1 |
| PART11 | 0.657 | 0.553322434 | 0.314 | 0.404 | 0.079 | 21 | PART1 |
| SLC31A2 | 0.655 | 0.6996389 | 0.31 | 0.367 | 0.053 | 21 | SLC31A2 |
| PLA2R1 | 0.652 | 0.611487915 | 0.304 | 0.385 | 0.076 | 21 | PLA2R1 |
| SLC5A81 | 0.651 | 0.426639371 | 0.302 | 0.468 | 0.141 | 21 | SLC5A8 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 2 (macrophage).

TABLE 2

| Pos Corr | Neg Corr |
|---|---|
| CCL18 | TANC1 |
| MARCO | PLD4 |
| PTAFR | C1orf63 |
| MS4A4A | ST3GAL6 |
| CCL13 | CA12 |
| CCNYL1 | APOPT1 |
| SLC2A3 | CACNA2D3 |
| FAM127A | USP15 |
| CLEC4G | TBC1D9 |
| LYVE1 | |
| TIMP1 | |
| RYK | |
| SYVN1 | |
| CTSL | |
| EP400 | |
| TMCO4 | |
| LILRB5 | |
| RNASE1 | |
| FFAR3 | |
| MAP1S | |
| ABL1 | |
| GBA | |
| MTRNR2L4 | |
| AP2A2 | |
| SLC39A8 | |
| CCRN4L | |
| FOLR2 | |
| C19orf59 | |
| SLC26A6 | |
| SLC26A11 | |
| PSMC1 | |
| DCLRE1A | |
| GZMA | |
| CCL23 | |
| ZNF320 | |
| MED22 | |
| NFATC1 | |
| CCL24 | |
| CD163 | |
| IMMT | |
| TICAM1 | |
| RAMP1 | |
| CEP97 | |
| SLC10A7 | |
| COL15A1 | |
| PLTP | |
| PLXNA1 | |
| ADCK3 | |
| TENC1 | |
| PTGR2 | |
| COG8 | |
| SLC12A4 | |
| C7 | |
| BEND5 | |
| ASB13 | |
| ATG2A | |
| CLEC10A | |
| GAS2L3 | |
| CLC | |
| CENPM | |
| BNIP3 | |
| ATRIP | |
| AAK1 | |
| HTT | |
| RGL1 | |
| INPP4B | |
| XYLT2 | |
| EYA2 | |
| SNUPN | |
| FN1 | |
| C5AR2 | |
| PGD1L | |
| QTRT1 | |
| TFIP11 | |
| ZNF626 | |
| VCAM1 | |
| PPP1R12B | |
| ZNF567 | |
| ZSCAN25 | |
| F13A1 | |
| CD1C | |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 3. (fibroblast).

TABLE 3

| Pos Corr | Neg Corr |
|---|---|
| ITGA8 | ABCA5 |
| SULF2 | COL11A1 |
| PAPPA | NUP133 |
| NNMT | KANK4 |
| RGS4 | PNISR |
| TRAPPC3L | PIP |
| SERPINE1 | BPIFA1 |
| TNFRSF12A | RHOJ |
| POSTN | ABCA8 |
| SLC39A6 | RIC3 |
| OXTR | FGFBP2 |
| FAM89A | ABCA3 |
| ACKR4 | IFI44L |
| CHRDL2 | C6orf58 |
| PLOD2 | BPIFB2 |
| COL16A1 | FOXO3 |
| EFEMP1 | CCBE1 |
| HIF1A | LAMB1 |
| ACY1 | SMAD3 |
| LYVE1 | ATP1A2 |
| MPP4 | FGF10 |
| CCL13 | FAM65C |
| SSH1 | ATF7IP |
| IRS1 | HUNK |
| LIMA1 | TENM1 |
| DLL4 | CLMN |
| RNU11 | MT-ATP6 |
| CALU | SPOPL |
| TPM4 | CNTN3 |
| DCBLD2 | PRB4 |
| DUSP5 | SLC18A2 |
| MYLK4 | |
| C19orf81 | |
| FTMT | |
| TMIE | |
| ZNF781 | |
| TNFSF18 | |
| FSTL3 | |
| LINC00152 | |
| CCL8 | |
| CMC2 | |
| DERL3 | |
| PCSK6 | |
| CYTH4 | |
| PRR16 | |
| AC002310.13 | |
| LINC00984 | |
| CDH13 | |
| RP11-417E7.1 | |
| SMOC1 | |
| IL6 | |
| AC002454.1 | |
| ZC3H12B | |
| RP11-798G7.6 | |
| INSRR | |
| CCR2 | |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 4.

TABLE 4

| | p_val | avg_diff | pct. 1 | pct. 2 |
|---|---|---|---|---|
| POSTN | 0 | 2.670251801 | 0.914 | 0.287 |
| PTHLH | 2.02E−274 | 1.829468067 | 0.45 | 0.053 |
| IGJ | 1.10E−246 | 1.581109351 | 0.526 | 0.162 |
| IGHG4 | 1.12E−138 | 1.546769308 | 0.233 | 0.029 |
| IGHA1 | 1.42E−223 | 1.435083651 | 0.566 | 0.3 |
| SERPINB2 | 2.83E−122 | 1.400940283 | 0.203 | 0.011 |
| MMP10 | 4.56E−197 | 1.383323268 | 0.611 | 0.293 |
| ALOX15 | 0 | 1.334962304 | 0.774 | 0.528 |
| CCL26 | 2.82E−88 | 1.258889137 | 0.172 | 0.021 |
| RPS4Y1 | 3.26E−132 | 1.067387658 | 0.179 | 0.003 |
| NTRK2 | 1.38E−110 | 1.058464811 | 0.292 | 0.079 |
| SERPINB10 | 4.82E−120 | 1.008503941 | 0.186 | 0.008 |
| CDH26 | 9.64E−91 | 0.945981339 | 0.212 | 0.035 |
| IGHA2 | 6.51E−72 | 0.911320044 | 0.211 | 0.057 |
| IGFBP3 | 4.11E−54 | 0.865094527 | 0.303 | 0.181 |
| MIF | 6.72E−117 | 0.863607558 | 0.388 | 0.219 |
| S100A2 | 1.72E−189 | 0.851057782 | 0.845 | 0.726 |
| SLC5A3 | 4.82E−63 | 0.815744371 | 0.19 | 0.051 |
| KCNJ16 | 6.49E−102 | 0.812721183 | 0.146 | 0.003 |
| FBXO32 | 5.75E−67 | 0.799202356 | 0.227 | 0.082 |
| IGHG1 | 2.05E−56 | 0.795484335 | 0.166 | 0.041 |
| HS3ST1 | 5.95E−81 | 0.777171859 | 0.372 | 0.248 |
| TNC | 1.05E−101 | 0.772635318 | 0.484 | 0.335 |
| CD44 | 1.12E−114 | 0.755812808 | 0.496 | 0.357 |
| FGFBP1 | 8.35E−49 | 0.732525838 | 0.137 | 0.035 |
| LGALS7 | 1.64E−52 | 0.717977188 | 0.232 | 0.114 |
| LAMB3 | 4.00E−69 | 0.716368572 | 0.386 | 0.233 |
| EGLN3 | 2.41E−58 | 0.710569297 | 0.154 | 0.032 |
| IFITM1 | 1.62E−65 | 0.708068639 | 0.237 | 0.125 |
| MRPS6 | 1.31E−62 | 0.697890556 | 0.273 | 0.134 |
| TFF3 | 1.61E−41 | 0.686701572 | 0.243 | 0.143 |
| IFITM3 | 1.73E−87 | 0.673023097 | 0.508 | 0.36 |
| SERPINF1 | 4.83E−92 | 0.663080187 | 0.488 | 0.358 |
| IGHG3 | 1.01E−46 | 0.656687479 | 0.163 | 0.081 |
| KRT17 | 4.94E−82 | 0.642721422 | 0.489 | 0.368 |
| SDPR | 2.61E−33 | 0.624582619 | 0.173 | 0.077 |
| TXNDC17 | 8.26E−82 | 0.619260035 | 0.427 | 0.334 |
| KRT15 | 1.80E−77 | 0.610462262 | 0.65 | 0.437 |
| IGHM | 2.62E−34 | 0.604716288 | 0.104 | 0.022 |
| CTSC | 3.85E−76 | 0.59257176 | 0.392 | 0.32 |
| KRT6A | 6.49E−31 | 0.591722184 | 0.225 | 0.144 |
| GPR155 | 5.25E−34 | 0.570178116 | 0.203 | 0.095 |
| MTRNR2L2 | 7.47E−58 | 0.566611234 | 0.752 | 0.66 |
| CTD-2228K2.5 | 8.10E−33 | 0.56285963 | 0.169 | 0.089 |
| LOXL4 | 1.34E−34 | 0.561605829 | 0.149 | 0.057 |
| TNFRSF12A | 1.34E−35 | 0.557462999 | 0.216 | 0.132 |
| CBR1 | 4.53E−61 | 0.557429291 | 0.382 | 0.299 |
| CAV1 | 1.02E−36 | 0.550314115 | 0.129 | 0.039 |
| KLF10 | 4.94E−69 | 0.549945474 | 0.416 | 0.357 |
| RPL18A | 3.21E−76 | 0.542774744 | 0.496 | 0.396 |
| IFITM2 | 4.19E−37 | 0.541028394 | 0.245 | 0.152 |
| STOM | 6.18E−37 | 0.539216798 | 0.238 | 0.153 |
| GPX4 | 3.47E−117 | 0.537021217 | 0.636 | 0.585 |
| SERPINE2 | 1.23E−37 | 0.53594894 | 0.115 | 0.029 |
| S100A10 | 2.33E−74 | 0.533923387 | 0.462 | 0.399 |
| CXCL2 | 3.57E−20 | 0.531928659 | 0.214 | 0.156 |
| NDRG1 | 1.21E−30 | 0.517371524 | 0.173 | 0.095 |
| CST1 | 2.24E−20 | 0.517156843 | 0.125 | 0.051 |
| SFN | 2.10E−65 | 0.514168056 | 0.406 | 0.348 |
| MYL9 | 4.72E−32 | 0.506849586 | 0.149 | 0.066 |
| FAM110C | 6.06E−34 | 0.506714921 | 0.106 | 0.024 |
| UBBP4 | 8.24E−44 | 0.506396458 | 0.279 | 0.202 |
| ELOVL5 | 4.56E−31 | 0.502885046 | 0.196 | 0.117 |
| HMGB3 | 2.91E−33 | 0.493181645 | 0.172 | 0.102 |
| PTRF | 1.17E−29 | 0.491227925 | 0.215 | 0.148 |
| IGFBP7 | 6.24E−40 | 0.484868652 | 0.355 | 0.287 |
| LDHA | 4.72E−57 | 0.4763654 | 0.399 | 0.336 |
| SELM | 9.14E−35 | 0.474684929 | 0.244 | 0.182 |
| MTRNR2L1 | 4.42E−137 | 0.473323453 | 0.914 | 0.755 |
| GPC3 | 9.89E−33 | 0.470951585 | 0.101 | 0.024 |
| RPL21 | 8.23E−54 | 0.468097689 | 0.337 | 0.281 |
| RPS27 | 7.12E−45 | 0.456451947 | 0.621 | 0.501 |
| NEDD9 | 1.57E−23 | 0.446826495 | 0.17 | 0.117 |
| CTNNAL1 | 5.94E−24 | 0.437053273 | 0.179 | 0.117 |
| RPS25 | 4.79E−95 | 0.428727348 | 0.833 | 0.778 |
| DLK2 | 8.21E−22 | 0.40968698 | 0.182 | 0.128 |
| PAPSS2 | 3.35E−23 | 0.407858974 | 0.122 | 0.056 |
| CCND2 | 4.13E−20 | 0.406490888 | 0.11 | 0.057 |
| MYO10 | 4.78E−20 | 0.405191878 | 0.115 | 0.055 |
| NOS2 | 1.04E−17 | 0.404862792 | 0.177 | 0.118 |
| LRRC17 | 1.43E−17 | 0.382198215 | 0.101 | 0.041 |
| RPL10 | 3.82E−39 | 0.372794137 | 0.656 | 0.565 |
| CKB | 3.74E−16 | 0.370394001 | 0.164 | 0.112 |
| DLL1 | 1.57E−13 | 0.360830301 | 0.136 | 0.073 |
| RPL13A | 6.65E−110 | 0.357093627 | 0.935 | 0.865 |
| RPL36 | 9.26E−38 | 0.302071424 | 0.677 | 0.623 |
| SKIL | 1.20E−32 | −0.250642674 | 0.122 | 0.245 |
| ODF2L | 3.68E−21 | −0.250893818 | 0.055 | 0.131 |
| CITED2 | 9.10E−30 | −0.251054227 | 0.109 | 0.223 |
| SLC5A8 | 9.60E−25 | −0.251429869 | 0.099 | 0.201 |
| SLITRK6 | 1.96E−19 | −0.25211946 | 0.068 | 0.149 |
| HLA-B | 1.19E−31 | −0.252273541 | 0.715 | 0.845 |
| DDIT3 | 2.46E−37 | −0.252563897 | 0.152 | 0.294 |
| CYP26A1 | 7.20E−20 | −0.254185392 | 0.035 | 0.101 |
| CLK1 | 1.41E−52 | −0.257804125 | 0.214 | 0.398 |
| SMARCA2 | 1.74E−29 | −0.257922321 | 0.125 | 0.246 |
| JAG1 | 5.24E−34 | −0.258040874 | 0.095 | 0.212 |
| PLCB4 | 1.26E−24 | −0.259048232 | 0.068 | 0.159 |
| CRNDE | 3.68E−29 | −0.259572499 | 0.032 | 0.112 |
| MAL2 | 1.22E−29 | −0.259881019 | 0.059 | 0.154 |
| ZNF428 | 1.46E−41 | −0.260286938 | 0.103 | 0.235 |
| ARG2 | 8.15E−26 | −0.260711917 | 0.057 | 0.145 |
| CFH | 4.23E−54 | −0.26171464 | 0.22 | 0.409 |
| ARSD | 2.24E−42 | −0.262061082 | 0.057 | 0.168 |
| COL4A3BP | 2.71E−39 | −0.2632293 | 0.118 | 0.253 |
| RHOBTB3 | 3.64E−22 | −0.263902254 | 0.046 | 0.123 |
| RP11-469H8.6 | 1.45E−29 | −0.265130963 | 0.085 | 0.193 |
| PARP14 | 1.15E−25 | −0.265213363 | 0.066 | 0.159 |
| IQGAP1 | 6.44E−48 | −0.265308753 | 0.269 | 0.459 |
| TCEA3 | 2.06E−32 | −0.267230684 | 0.044 | 0.136 |
| PARVA | 1.45E−31 | −0.267998646 | 0.066 | 0.169 |
| DCXR | 5.25E−33 | −0.268186829 | 0.096 | 0.214 |
| CTSL | 2.52E−32 | −0.268653343 | 0.064 | 0.167 |
| NQO1 | 1.02E−31 | −0.269495928 | 0.054 | 0.152 |
| PTK2 | 1.11E−24 | −0.270864513 | 0.087 | 0.187 |
| RPL39L | 2.99E−14 | −0.271582621 | 0.075 | 0.148 |
| NBPF10 | 5.46E−26 | −0.27425596 | 0.03 | 0.104 |
| MTRNR2L5 | 1.70E−09 | −0.274320583 | 0.146 | 0.211 |
| FAM84A | 4.14E−28 | −0.276412995 | 0.035 | 0.117 |
| NR4A2 | 2.16E−25 | −0.276881912 | 0.145 | 0.265 |
| LGALS8 | 1.61E−48 | −0.27846316 | 0.16 | 0.325 |
| MT1G | 3.29E−33 | −0.278869878 | 0.031 | 0.117 |
| SVIP | 3.02E−32 | −0.279839074 | 0.058 | 0.158 |
| TNFSF10 | 3.51E−52 | −0.281493639 | 0.228 | 0.419 |
| RNF19A | 4.85E−36 | −0.28167473 | 0.095 | 0.219 |
| ZNF703 | 9.34E−30 | −0.282215484 | 0.032 | 0.115 |
| MUC1 | 4.37E−24 | −0.282265729 | 0.051 | 0.135 |
| DAPL1 | 1.02E−39 | −0.282575936 | 0.052 | 0.159 |
| TPD52L1 | 3.48E−31 | −0.283928659 | 0.11 | 0.232 |
| NDRG2 | 1.65E−36 | −0.284165661 | 0.072 | 0.188 |
| MTRNR2L6 | 1.63E−16 | −0.28431922 | 0.163 | 0.256 |
| TET2 | 9.40E−32 | −0.286040253 | 0.05 | 0.147 |
| PRSS23 | 4.77E−62 | −0.286752037 | 0.636 | 0.848 |
| ADAM28 | 8.21E−57 | −0.290189452 | 0.246 | 0.451 |
| NEAT1 | 1.49E−25 | −0.290499316 | 0.197 | 0.328 |
| SLC15A2 | 4.33E−38 | −0.294154781 | 0.085 | 0.208 |
| TSHZ2 | 1.10E−39 | −0.297202871 | 0.083 | 0.206 |
| ADRB2 | 4.63E−36 | −0.297591798 | 0.067 | 0.179 |
| RCBTB1 | 6.17E−32 | −0.299260742 | 0.054 | 0.153 |
| STAT1 | 1.35E−36 | −0.299878042 | 0.066 | 0.18 |
| RARRES3 | 2.69E−34 | −0.300477919 | 0.057 | 0.163 |
| LSAMP | 2.89E−25 | −0.3032095 | 0.047 | 0.133 |
| AKR1A1 | 2.13E−44 | −0.303626082 | 0.158 | 0.32 |
| IGFBP5 | 9.77E−21 | −0.303923471 | 0.087 | 0.179 |
| PBX1 | 3.55E−52 | −0.304384871 | 0.103 | 0.254 |
| SEMA3C | 4.20E−29 | −0.305066223 | 0.075 | 0.18 |
| SLC31A1 | 2.33E−40 | −0.30597278 | 0.077 | 0.202 |
| RBM5 | 9.33E−27 | −0.308210224 | 0.087 | 0.194 |
| STRBP | 6.49E−32 | −0.308666156 | 0.042 | 0.137 |
| HLA-A | 5.44E−36 | −0.310340061 | 0.494 | 0.674 |
| HPGD | 7.81E−39 | −0.31339638 | 0.237 | 0.406 |
| ATP1B1 | 6.22E−46 | −0.314066994 | 0.377 | 0.583 |
| MGEA5 | 2.01E−36 | −0.314722767 | 0.078 | 0.198 |
| PTPN13 | 1.70E−52 | −0.318651108 | 0.16 | 0.337 |
| SLC9A3R1 | 7.12E−52 | −0.320005418 | 0.165 | 0.343 |
| B4GALT5 | 3.36E−37 | −0.320098137 | 0.032 | 0.127 |
| BTG1 | 3.90E−56 | −0.320247106 | 0.292 | 0.509 |
| CFD | 1.03E−17 | −0.321833872 | 0.036 | 0.101 |
| EMP2 | 5.57E−52 | −0.32263391 | 0.101 | 0.253 |
| CMPK1 | 8.06E−45 | −0.3272815 | 0.136 | 0.295 |
| CLEC2B | 2.88E−43 | −0.327512252 | 0.175 | 0.344 |
| PHLDA2 | 3.34E−37 | −0.329148167 | 0.125 | 0.267 |
| EPHX1 | 8.11E−63 | −0.333683517 | 0.141 | 0.327 |
| CLINT1 | 1.14E−53 | −0.336788092 | 0.197 | 0.39 |
| CCND1 | 1.81E−56 | −0.337502247 | 0.191 | 0.387 |
| S100A9 | 2.99E−33 | −0.340863333 | 0.103 | 0.226 |
| SELENBP1 | 3.64E−48 | −0.34199207 | 0.066 | 0.201 |
| PPDPF | 2.17E−51 | −0.342809978 | 0.25 | 0.455 |
| CTSD | 5.64E−65 | −0.346258074 | 0.337 | 0.579 |
| GK5 | 9.67E−37 | −0.346869771 | 0.026 | 0.117 |
| XBP1 | 1.34E−50 | −0.347740992 | 0.234 | 0.433 |
| SREBF1 | 1.33E−45 | −0.348816976 | 0.031 | 0.139 |
| EPCAM | 3.63E−34 | −0.352887738 | 0.061 | 0.172 |
| GGT6 | 7.26E−51 | −0.353107545 | 0.029 | 0.143 |
| SERPINB1 | 9.05E−47 | −0.354877342 | 0.133 | 0.296 |
| ANK3 | 9.57E−39 | −0.359661825 | 0.126 | 0.275 |
| DUSP2 | 4.17E−35 | −0.359739096 | 0.159 | 0.308 |
| CYP2J2 | 1.04E−36 | −0.362122427 | 0.033 | 0.131 |
| ID2 | 8.12E−55 | −0.362372948 | 0.263 | 0.477 |
| TOB1 | 3.86E−64 | −0.367315495 | 0.346 | 0.589 |
| NUPR1 | 4.32E−60 | −0.367407669 | 0.251 | 0.472 |
| COBLL1 | 6.82E−36 | −0.36740801 | 0.03 | 0.125 |
| SCNN1A | 5.43E−49 | −0.369565577 | 0.103 | 0.26 |
| SCIN | 3.48E−44 | −0.371158916 | 0.03 | 0.137 |
| PVRL4 | 1.25E−48 | −0.371264794 | 0.014 | 0.111 |
| RNF152 | 2.48E−43 | −0.371487374 | 0.037 | 0.148 |
| MGST1 | 4.54E−53 | −0.374140135 | 0.243 | 0.453 |
| COLCA1 | 5.50E−51 | −0.375451479 | 0.019 | 0.127 |
| RTN4 | 1.16E−54 | −0.375613472 | 0.178 | 0.373 |
| GCHFR | 6.00E−51 | −0.375648448 | 0.087 | 0.24 |
| IL18 | 1.04E−54 | −0.375688864 | 0.07 | 0.219 |
| ELF3 | 9.56E−54 | −0.379372753 | 0.453 | 0.677 |
| RNA18S5 | 3.11E−19 | −0.382358319 | 0.09 | 0.18 |
| S100A14 | 4.03E−54 | −0.386601686 | 0.085 | 0.244 |
| SNHG8 | 4.24E−64 | −0.386819529 | 0.329 | 0.573 |
| MT1X | 1.96E−94 | −0.391776317 | 0.415 | 0.704 |
| APOD | 1.08E−42 | −0.391877897 | 0.022 | 0.12 |
| CD74 | 1.04E−32 | −0.392636093 | 0.221 | 0.383 |
| EXPH5 | 4.37E−51 | −0.39348639 | 0.132 | 0.307 |
| MKL2 | 5.18E−64 | −0.396799117 | 0.108 | 0.29 |
| HEY1 | 2.16E−47 | −0.400908816 | 0.022 | 0.128 |
| PRDX6 | 3.01E−70 | −0.401714585 | 0.216 | 0.448 |
| IFI27 | 2.12E−34 | −0.402760185 | 0.194 | 0.356 |
| TFCP2L1 | 7.43E−62 | −0.403192374 | 0.081 | 0.245 |
| SAMHD1 | 2.78E−55 | −0.417585661 | 0.027 | 0.15 |
| TRIM24 | 6.62E−40 | −0.418807555 | 0.068 | 0.197 |
| RHOV | 1.43E−53 | −0.422570503 | 0.021 | 0.137 |
| ZFP36L1 | 1.65E−91 | −0.423470376 | 0.559 | 0.825 |
| EGFR | 1.39E−61 | −0.425401887 | 0.213 | 0.434 |
| PRKAR2B | 2.42E−57 | −0.425530306 | 0.007 | 0.107 |
| SAT1 | 4.22E−73 | −0.426377595 | 0.639 | 0.848 |
| GABRP | 9.00E−61 | −0.429145335 | 0.146 | 0.345 |
| EFCAB4A | 9.76E−58 | −0.430676956 | 0.008 | 0.111 |
| PLAC8 | 2.98E−46 | −0.437800139 | 0.052 | 0.183 |
| HNMT | 3.17E−68 | −0.443282392 | 0.028 | 0.167 |
| CLDN7 | 2.03E−63 | −0.447207131 | 0.095 | 0.277 |
| KRT19 | 1.10E−110 | −0.450540066 | 0.854 | 0.967 |
| ABCA13 | 2.87E−55 | −0.451262596 | 0.074 | 0.231 |
| PIK3R3 | 1.25E−52 | −0.452371695 | 0.062 | 0.21 |
| ACAP2 | 4.66E−66 | −0.452574103 | 0.166 | 0.384 |
| ZNF750 | 5.68E−58 | −0.45360995 | 0.023 | 0.146 |
| NEBL | 1.96E−55 | −0.458356225 | 0.025 | 0.145 |
| CLDN4 | 3.29E−45 | −0.46543423 | 0.474 | 0.664 |
| PAX7 | 1.58E−65 | −0.466613863 | 0.017 | 0.144 |
| SEMA3A | 1.17E−66 | −0.467395055 | 0.005 | 0.111 |
| DUSP1 | 6.60E−80 | −0.471882632 | 0.747 | 0.9 |
| OGFRL1 | 4.62E−61 | −0.472343066 | 0.076 | 0.246 |
| CD82 | 7.76E−69 | −0.47285878 | 0.119 | 0.324 |
| RIMS1 | 2.52E−67 | −0.480377336 | 0.027 | 0.166 |
| SLPI | 9.37E−82 | −0.48108 | 0.84 | 0.944 |
| GLUL | 8.68E−96 | −0.483181151 | 0.504 | 0.788 |
| MPV17L | 2.89E−68 | −0.487184457 | 0.052 | 0.216 |
| KIF21A | 4.10E−75 | −0.48848067 | 0.107 | 0.315 |
| ERN2 | 4.33E−61 | −0.497445718 | 0.012 | 0.125 |
| ALDH2 | 1.13E−88 | −0.498591892 | 0.165 | 0.418 |
| DDIT4 | 6.03E−89 | −0.50317121 | 0.196 | 0.462 |
| SULT1E1 | 8.19E−45 | −0.506877661 | 0.056 | 0.188 |
| FAM3B | 1.45E−68 | −0.510615336 | 0.075 | 0.258 |
| CA12 | 4.91E−82 | −0.512556499 | 0.09 | 0.298 |
| CALML3 | 5.07E−52 | −0.51633447 | 0.014 | 0.117 |
| MTRNR2L11 | 1.09E−20 | −0.521941303 | 0.165 | 0.251 |
| HSPB1 | 4.38E−84 | −0.523559754 | 0.532 | 0.78 |
| TMPRSS11D | 6.42E−77 | −0.528459418 | 0.046 | 0.22 |
| FAM3D | 5.51E−83 | −0.539626075 | 0.009 | 0.147 |
| CTSB | 3.65E−106 | −0.543522005 | 0.427 | 0.73 |
| AQP3 | 1.05E−110 | −0.544508011 | 0.712 | 0.906 |
| HES1 | 3.89E−78 | −0.555167805 | 0.438 | 0.697 |
| ANXA1 | 1.89E−114 | −0.556630316 | 0.709 | 0.922 |
| LTF | 4.98E−102 | −0.558232928 | 0.016 | 0.187 |
| AGR2 | 3.02E−140 | −0.5898891 | 0.41 | 0.766 |
| XIST | 6.97E−96 | −0.593870589 | 0.179 | 0.454 |
| CXCL17 | 7.60E−85 | −0.595041921 | 0.17 | 0.427 |
| ALDH3A1 | 1.23E−84 | −0.597428673 | 0.405 | 0.677 |
| ALCAM | 2.35E−90 | −0.60456057 | 0.226 | 0.507 |
| AQP5 | 3.10E−92 | −0.610484799 | 0.13 | 0.382 |
| ALDH1A1 | 5.11E−104 | −0.611733656 | 0.342 | 0.654 |
| FMO3 | 1.02E−84 | −0.638489162 | 0.146 | 0.394 |
| WFDC2 | 1.95E−87 | −0.649526386 | 0.31 | 0.593 |
| PTN | 1.73E−90 | −0.654993061 | 0.06 | 0.271 |
| KRT8 | 2.90E−104 | −0.662271996 | 0.397 | 0.69 |
| SPINK5 | 4.48E−44 | −0.662944885 | 0.117 | 0.272 |
| TSPAN1 | 4.30E−85 | −0.673580308 | 0.13 | 0.375 |
| KRT7 | 4.59E−97 | −0.693470573 | 0.112 | 0.367 |
| FN1 | 8.46E−61 | −0.701011398 | 0.013 | 0.126 |
| AKR1C2 | 4.03E−101 | −0.709595229 | 0.04 | 0.247 |
| CSRP2 | 4.15E−102 | −0.710031741 | 0.068 | 0.304 |
| CYP4B1 | 1.45E−100 | −0.720418734 | 0.132 | 0.404 |
| NTS | 2.83E−64 | −0.723003952 | 0.216 | 0.443 |
| NET1 | 5.06E−114 | −0.727055801 | 0.137 | 0.429 |
| PTGS2 | 7.04E−68 | −0.735239021 | 0.024 | 0.167 |
| UGT2A2 | 1.96E−123 | −0.747720352 | 0.299 | 0.633 |
| CHP2 | 6.95E−94 | −0.754460116 | 0.026 | 0.207 |
| C8orf4 | 2.24E−72 | −0.774174949 | 0.064 | 0.251 |
| FAM107A | 6.74E−134 | −0.789879063 | 0.062 | 0.337 |
| BPIFB1 | 2.31E−178 | −0.80744545 | 0.112 | 0.471 |
| CLCA4 | 5.56E−102 | −0.833589812 | 0.019 | 0.203 |
| MSMB | 6.42E−98 | −0.856796467 | 0.066 | 0.294 |
| GDF15 | 5.25E−79 | −0.919009157 | 0.032 | 0.198 |
| S100A4 | 4.86E−106 | −0.932849307 | 0.135 | 0.412 |
| TMEM213 | 1.33E−109 | −0.943877366 | 0.018 | 0.209 |
| AKR1C3 | 3.65E−161 | −0.994939846 | 0.059 | 0.367 |
| BPIFA1 | 7.43E−280 | −1.062354514 | 0.181 | 0.68 |
| PIGR | 2.40E−150 | −1.145841345 | 0.063 | 0.362 |
| ZG16B | 6.08E−245 | −1.147688517 | 0.014 | 0.357 |
| EPAS1 | 0 | −1.269843585 | 0.305 | 0.818 |
| SCGB1A1 | 1.59E−124 | −1.351785509 | 0.024 | 0.244 |
| LYZ | 5.63E−287 | −1.452623139 | 0.033 | 0.452 |
| STATH | 0 | −2.670221426 | 0.029 | 0.835 |

In certain example embodiments, the biomarkers for detection of cell phenotypes and/or targets for modulating endothelial cell proliferation, differentiation, maintenance and/or function comprising modulating one or more genes or gene expression products in Table 5.

TABLE 5

| | p_val | avg_diff | pct. 1 | pct. 2 |
|---|---|---|---|---|
| STATH | 4.76E−205 | −3.168571138 | 0.046 | 0.883 |
| LYZ | 9.45E−83 | −1.597097241 | 0.055 | 0.564 |
| BPIFA1 | 4.01E−71 | −0.905538391 | 0.24 | 0.762 |
| ZG16B | 1.35E−65 | −1.45123357 | 0.012 | 0.382 |
| IGJ | 5.93E−44 | 1.201131086 | 0.59 | 0.217 |
| LTF | 8.63E−44 | −0.77407133 | 0.023 | 0.294 |
| ADIRF | 8.33E−41 | −0.775869943 | 0.499 | 0.844 |
| PLAT | 2.82E−40 | −1.359479676 | 0.053 | 0.37 |
| CLU | 7.08E−40 | −1.160253935 | 0.373 | 0.718 |
| POSTN | 1.57E−39 | 1.125308122 | 0.684 | 0.467 |
| BPIFB1 | 2.74E−36 | −0.686504435 | 0.152 | 0.499 |
| PTGDS | 1.33E−34 | −1.042731548 | 0.014 | 0.238 |
| RPS4Y1 | 2.45E−34 | 1.323066763 | 0.223 | 0.005 |
| MSMB | 1.47E−33 | −0.935112303 | 0.033 | 0.287 |
| SLPI | 6.88E−33 | −0.423726797 | 0.439 | 0.798 |
| IGHG4 | 1.37E−30 | 2.331879835 | 0.316 | 0.078 |
| HLA-A | 2.14E−30 | −0.523215287 | 0.648 | 0.903 |
| IGHA1 | 7.03E−30 | 0.76312007 | 0.631 | 0.418 |
| DNASE1L3 | 1.28E−29 | −1.205812239 | 0.033 | 0.268 |
| CLEC3B | 5.57E−27 | −0.870610999 | 0.015 | 0.204 |
| VIM | 2.09E−23 | −0.394979913 | 0.884 | 0.976 |
| XIST | 4.24E−23 | −0.691268117 | 0.18 | 0.472 |
| EDN1 | 1.83E−21 | −0.918666342 | 0.086 | 0.321 |
| ENG | 7.86E−20 | −0.538774523 | 0.246 | 0.526 |
| SEPP1 | 2.91E−19 | −0.52586809 | 0.277 | 0.56 |
| NET1 | 3.34E−19 | −0.457046581 | 0.157 | 0.397 |
| ABCB1 | 9.01E−19 | −0.591960673 | 0.143 | 0.384 |
| CLDN5 | 2.93E−18 | 0.716302515 | 0.661 | 0.479 |
| PIGR | 6.72E−18 | −0.569946395 | 0.07 | 0.26 |
| MDK | 5.38E−17 | −0.403057392 | 0.173 | 0.394 |
| AHNAK | 6.70E−17 | −0.37629268 | 0.343 | 0.606 |
| ITM2A | 3.58E−16 | −0.276950384 | 0.234 | 0.453 |
| ITM2B | 2.00E−15 | −0.354440116 | 0.738 | 0.903 |
| GIMAP7 | 2.10E−15 | −0.432676003 | 0.384 | 0.642 |
| IGHG3 | 2.30E−15 | 1.208980607 | 0.236 | 0.092 |
| HLA-B | 2.62E−15 | −0.293414099 | 0.891 | 0.968 |
| SYNE1 | 2.70E−15 | −0.664625155 | 0.072 | 0.251 |
| NUPR1 | 3.55E−15 | −0.405294708 | 0.115 | 0.297 |
| TXNIP | 4.20E−15 | −0.317420415 | 0.649 | 0.869 |
| GIMAP5 | 4.27E−15 | −0.487546137 | 0.092 | 0.275 |
| TNFSF10 | 9.39E−15 | −0.340997924 | 0.306 | 0.543 |
| LIFR | 1.32E−14 | −0.380044824 | 0.309 | 0.55 |
| HLA-E | 1.96E−14 | −0.253676861 | 0.895 | 0.978 |
| CAV1 | 7.87E−14 | −0.490960829 | 0.333 | 0.577 |
| CXCL12 | 9.06E−14 | −0.614587187 | 0.153 | 0.363 |
| NRP2 | 1.03E−13 | −0.315571593 | 0.075 | 0.219 |
| ENAH | 1.26E−13 | −0.353942709 | 0.081 | 0.234 |
| APOL3 | 1.67E−13 | −0.403572921 | 0.09 | 0.253 |
| LPAR6 | 1.86E−13 | −0.386749152 | 0.246 | 0.47 |
| MX1 | 3.83E−13 | −0.553277519 | 0.068 | 0.229 |
| LGALS3 | 4.03E−13 | −0.314978021 | 0.594 | 0.813 |
| IFI44L | 4.15E−13 | −0.563426575 | 0.104 | 0.285 |
| MIF | 4.38E−13 | 0.524996339 | 0.346 | 0.273 |
| ID2 | 4.61E−13 | −0.373455196 | 0.205 | 0.401 |
| RPL7 | 5.81E−13 | 0.503235245 | 0.344 | 0.282 |
| IGFBP3 | 7.19E−13 | 1.177320433 | 0.224 | 0.085 |
| PSAP | 1.05E−12 | −0.286412574 | 0.433 | 0.662 |
| SPTBN1 | 1.71E−12 | −0.380201353 | 0.342 | 0.567 |
| CDKN1A | 1.89E−12 | 0.573555361 | 0.545 | 0.421 |
| VWF | 2.21E−12 | −0.437780466 | 0.391 | 0.623 |
| GLUL | 2.81E−12 | −0.435153185 | 0.253 | 0.472 |
| SYPL1 | 4.12E−12 | −0.354854563 | 0.149 | 0.321 |
| SPTAN1 | 4.35E−12 | −0.292980576 | 0.089 | 0.214 |
| ISG15 | 4.61E−12 | −0.366344916 | 0.096 | 0.251 |
| S100A2 | 4.73E−12 | 1.024811004 | 0.242 | 0.083 |
| SDPR | 8.35E−12 | −0.331943535 | 0.26 | 0.462 |
| GPR126 | 1.04E−11 | −0.470961159 | 0.07 | 0.217 |
| TIMP1 | 1.21E−11 | −0.319621574 | 0.262 | 0.462 |
| LY6E | 1.33E−11 | −0.487413513 | 0.145 | 0.331 |
| IGHA2 | 1.36E−11 | 1.274729816 | 0.262 | 0.102 |
| SSFA2 | 1.59E−11 | −0.275272271 | 0.208 | 0.389 |
| SERPING1 | 1.73E−11 | −0.354337153 | 0.078 | 0.214 |
| SYT15 | 2.14E−11 | −0.283751758 | 0.096 | 0.236 |
| FXYD5 | 2.21E−11 | −0.326344337 | 0.146 | 0.316 |
| SLC25A25 | 2.55E−11 | 0.529081283 | 0.306 | 0.243 |
| FOXP1 | 3.10E−11 | −0.414310403 | 0.223 | 0.426 |
| GPM6A | 3.33E−11 | −0.476154031 | 0.072 | 0.219 |
| CD74 | 3.47E−11 | −0.297495153 | 0.817 | 0.934 |
| GBP4 | 3.51E−11 | −0.379299061 | 0.134 | 0.302 |
| CTSC | 5.21E−11 | −0.378097193 | 0.25 | 0.453 |
| VAT1 | 8.67E−11 | −0.253439861 | 0.111 | 0.251 |
| MTRNR2L2 | 1.22E−10 | 0.451017925 | 0.742 | 0.628 |
| SAT1 | 1.67E−10 | −0.316120258 | 0.441 | 0.647 |
| BST2 | 2.18E−10 | −0.392157503 | 0.153 | 0.321 |
| SYNE2 | 2.28E−10 | −0.332491273 | 0.359 | 0.567 |
| CALCOCO2 | 3.01E−10 | −0.350544343 | 0.164 | 0.333 |
| TOMM7 | 3.36E−10 | −0.265172803 | 0.447 | 0.652 |
| IGKC | 3.56E−10 | −0.54312598 | 0.094 | 0.226 |
| AKR1C3 | 3.66E−10 | −0.318351215 | 0.163 | 0.328 |
| IFIT1 | 4.17E−10 | −0.474058605 | 0.081 | 0.224 |
| PPDPF | 6.21E−10 | −0.292268183 | 0.19 | 0.353 |
| TSC22D1 | 6.55E−10 | −0.361240757 | 0.396 | 0.603 |
| PPAP2A | 6.61E−10 | −0.449399286 | 0.117 | 0.275 |
| LYST | 8.38E−10 | −0.405696638 | 0.101 | 0.248 |
| HLA-DQA1 | 8.89E−10 | −0.273957495 | 0.149 | 0.297 |
| WFDC2 | 1.39E−09 | −0.414449294 | 0.135 | 0.29 |
| YBX3 | 1.76E−09 | 0.452477779 | 0.392 | 0.341 |
| CLEC14A | 1.77E−09 | −0.301412799 | 0.239 | 0.411 |
| SELE | 1.87E−09 | −0.573381618 | 0.245 | 0.426 |
| HLA-DPB1 | 1.94E−09 | −0.280498294 | 0.305 | 0.491 |
| PRCP | 2.20E−09 | −0.389244656 | 0.275 | 0.467 |
| TMEM70 | 2.23E−09 | 0.552632086 | 0.276 | 0.19 |
| HLA-C | 2.39E−09 | −0.298137135 | 0.745 | 0.866 |
| UACA | 4.19E−09 | −0.297635376 | 0.227 | 0.397 |
| MGP | 4.32E−09 | −0.576727946 | 0.302 | 0.487 |
| PLS3 | 5.50E−09 | −0.266594997 | 0.16 | 0.307 |
| ABCG2 | 5.64E−09 | −0.350703427 | 0.109 | 0.243 |
| NFE2L1 | 5.67E−09 | −0.28430244 | 0.163 | 0.314 |
| BMPR2 | 5.74E−09 | −0.288159066 | 0.189 | 0.348 |
| PJA2 | 6.30E−09 | −0.315563268 | 0.124 | 0.265 |
| C10orf10 | 6.88E−09 | −0.567159336 | 0.115 | 0.263 |
| TNFRSF14 | 7.46E−09 | −0.323645301 | 0.086 | 0.207 |
| SLC6A6 | 7.90E−09 | −0.4190738 | 0.081 | 0.212 |
| C7 | 1.09E−08 | −0.648491315 | 0.123 | 0.27 |
| SNHG7 | 1.13E−08 | −0.299725321 | 0.107 | 0.236 |
| SLC12A2 | 1.14E−08 | −0.381899966 | 0.096 | 0.229 |
| HSPA5 | 1.42E−08 | 0.461395655 | 0.395 | 0.345 |
| GPRC5B | 1.73E−08 | −0.343486475 | 0.119 | 0.26 |
| LAMP1 | 2.80E−08 | −0.319994849 | 0.104 | 0.234 |
| MT-RNR1 | 3.05E−08 | −0.252267051 | 0.858 | 0.949 |
| MT-ND2 | 3.08E−08 | −0.306953914 | 0.49 | 0.669 |
| IFI6 | 3.21E−08 | −0.400768668 | 0.126 | 0.27 |
| ASAP1 | 3.42E−08 | −0.325135108 | 0.131 | 0.273 |
| NPC2 | 3.87E−08 | −0.332833423 | 0.41 | 0.596 |
| MEF2A | 4.33E−08 | −0.372689971 | 0.173 | 0.331 |
| LIMCH1 | 5.07E−08 | −0.278934419 | 0.092 | 0.207 |
| SLFN5 | 5.71E−08 | −0.254201586 | 0.243 | 0.401 |
| CPE | 6.16E−08 | −0.394078099 | 0.089 | 0.212 |
| EPHX1 | 6.48E−08 | −0.301103066 | 0.092 | 0.214 |
| STAT1 | 7.52E−08 | −0.307450775 | 0.087 | 0.204 |
| DDIT4 | 1.11E−07 | −0.359756932 | 0.232 | 0.394 |
| SLC9A3R2 | 1.14E−07 | −0.402388198 | 0.096 | 0.219 |
| IQGAP1 | 1.76E−07 | −0.302892652 | 0.314 | 0.487 |
| LEPR | 2.36E−07 | −0.261858931 | 0.108 | 0.226 |
| TAGLN | 2.79E−07 | −0.37837418 | 0.116 | 0.238 |
| FAM198B | 3.85E−07 | −0.36837194 | 0.183 | 0.331 |
| NFIB | 3.85E−07 | −0.254033808 | 0.27 | 0.428 |
| GIMAP6 | 5.42E−07 | −0.347828051 | 0.123 | 0.251 |
| RAPGEF5 | 5.88E−07 | −0.275050714 | 0.098 | 0.212 |
| PLA2G16 | 6.79E−07 | −0.323189724 | 0.104 | 0.217 |
| HIF1A | 7.40E−07 | 0.491907973 | 0.227 | 0.163 |
| ARL6IP1 | 1.58E−06 | −0.364298561 | 0.135 | 0.265 |
| UBBP4 | 1.65E−06 | 0.46150148 | 0.295 | 0.202 |
| CDC42EP3 | 1.87E−06 | 0.409355217 | 0.286 | 0.229 |
| PODXL | 2.13E−06 | −0.315938243 | 0.197 | 0.338 |
| NFIA | 2.19E−06 | −0.269674094 | 0.102 | 0.209 |
| EMP1 | 2.43E−06 | 0.395240565 | 0.585 | 0.516 |
| HMGN2 | 2.51E−06 | −0.330233276 | 0.109 | 0.219 |
| TIMP3 | 2.98E−06 | −0.410182527 | 0.287 | 0.44 |
| TJP1 | 3.06E−06 | −0.347475963 | 0.18 | 0.319 |
| KIAA1551 | 4.20E−06 | −0.264932306 | 0.128 | 0.241 |
| SERINC1 | 5.91E−06 | −0.271492263 | 0.201 | 0.336 |
| GBP2 | 6.14E−06 | −0.30680091 | 0.141 | 0.263 |
| TFPI | 6.43E−06 | −0.270131145 | 0.175 | 0.299 |
| SPRY1 | 9.30E−06 | 0.420056958 | 0.423 | 0.338 |
| KDR | 9.81E−06 | −0.312678248 | 0.154 | 0.277 |
| CYSTM1 | 1.13E−05 | −0.271044093 | 0.127 | 0.241 |
| GPR116 | 3.92E−05 | −0.279723631 | 0.134 | 0.243 |
| DYNC1H1 | 0.00019751 | −0.315253079 | 0.119 | 0.219 |
| NKTR | 0.000845278 | −0.288868258 | 0.149 | 0.243 |
| CSRNP1 | 0.001301778 | 0.344025031 | 0.213 | 0.151 |
| MTRNR2L11 | 0.001777902 | −0.476677028 | 0.15 | 0.212 |
| S100A4 | 0.007657263 | −0.261108577 | 0.158 | 0.241 |
| ZNF90 | 0.010167421 | 0.280848505 | 0.246 | 0.187 |
| PNISR | 0.012503339 | −0.279859952 | 0.138 | 0.207 |
| MTRNR2L6 | 0.019607689 | −0.325823144 | 0.197 | 0.263 |
| MTRNR2L5 | 0.033940627 | −0.330661288 | 0.157 | 0.212 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 6.

TABLE 6

| | p_val | avg_diff | pct. 1 | pct. 2 |
|---|---|---|---|---|
| STATH | 1.01E−295 | −3.304933983 | 0.019 | 0.835 |
| POSTN | 9.94E−187 | 1.630482017 | 0.957 | 0.532 |
| BPIFA1 | 4.87E−112 | −1.800539141 | 0.12 | 0.656 |
| LYZ | 4.19E−102 | −1.4806442 | 0.025 | 0.435 |
| ZG16B | 7.86E−89 | −1.476446306 | 0.001 | 0.305 |
| SLPI | 3.67E−80 | −1.527882203 | 0.244 | 0.707 |
| TMSB4X | 1.18E−65 | 0.676001765 | 0.906 | 0.803 |
| IGFBP5 | 4.10E−63 | −1.188343019 | 0.183 | 0.594 |
| NNMT | 8.74E−54 | 1.24483343 | 0.554 | 0.203 |
| LGALS1 | 2.77E−53 | 0.675688886 | 0.851 | 0.746 |
| MT-ND2 | 6.80E−53 | −0.876347918 | 0.448 | 0.792 |
| IGJ | 8.08E−50 | 1.630769558 | 0.528 | 0.188 |
| BPIFB1 | 6.50E−47 | −1.446750573 | 0.097 | 0.402 |
| MTRNR2L12 | 1.82E−44 | −0.94173896 | 0.539 | 0.773 |
| PIGR | 2.41E−44 | −1.31932837 | 0.032 | 0.271 |
| MSMB | 4.44E−44 | −1.54822361 | 0.014 | 0.216 |
| IGHA1 | 7.67E−42 | 1.417576371 | 0.562 | 0.314 |
| IGHG4 | 1.37E−41 | 2.376231602 | 0.259 | 0.024 |
| FTH1 | 6.56E−38 | 0.56224292 | 0.797 | 0.737 |
| MTRNR2L6 | 3.10E−37 | −0.815513497 | 0.1 | 0.366 |
| CFI | 8.69E−36 | 1.148351121 | 0.3 | 0.064 |
| LIMA1 | 4.42E−34 | 0.682846632 | 0.606 | 0.475 |
| MT-CYB | 1.24E−33 | −0.583769857 | 0.455 | 0.762 |
| CCL26 | 3.42E−33 | 1.611798689 | 0.236 | 0.026 |
| RPL41 | 3.48E−33 | 0.461159479 | 0.852 | 0.777 |
| SLC18A2 | 1.13E−32 | −1.088382857 | 0.025 | 0.207 |
| SRGN | 7.21E−32 | 0.973805959 | 0.465 | 0.19 |
| FTL | 3.90E−31 | 0.504635167 | 0.877 | 0.824 |
| RGS2 | 6.63E−31 | 1.230967399 | 0.389 | 0.155 |
| FN1 | 6.82E−31 | 1.034609499 | 0.397 | 0.143 |
| SPARCL1 | 8.99E−31 | −0.875647789 | 0.076 | 0.303 |
| IFITM3 | 2.34E−30 | 0.502428075 | 0.791 | 0.68 |
| TMSB10 | 3.09E−30 | 0.462710283 | 0.835 | 0.764 |
| MYL6 | 3.53E−30 | 0.461645131 | 0.81 | 0.748 |
| MT-CO3 | 3.59E−30 | −0.634755864 | 0.507 | 0.775 |
| AEBP1 | 4.08E−30 | 0.758932888 | 0.58 | 0.439 |
| MT-ND4 | 1.26E−29 | −0.613893143 | 0.425 | 0.713 |
| MT-RNR1 | 1.16E−28 | −0.523486962 | 0.804 | 0.94 |
| XIST | 1.24E−28 | −0.753303173 | 0.195 | 0.463 |
| RARRES1 | 7.26E−28 | 1.015214947 | 0.493 | 0.378 |
| MT-ND1 | 3.93E−27 | −0.735000983 | 0.268 | 0.541 |
| MT-ND3 | 1.07E−26 | −0.682912352 | 0.318 | 0.594 |
| ALDH1A1 | 1.66E−26 | −0.744921595 | 0.09 | 0.298 |
| THBS1 | 1.89E−26 | 0.917089883 | 0.475 | 0.276 |
| SOCS3 | 2.48E−26 | 0.834838936 | 0.443 | 0.252 |
| SERF2 | 1.05E−25 | 0.495904874 | 0.675 | 0.589 |
| MTRNR2L11 | 5.00E−25 | −0.823003108 | 0.105 | 0.318 |
| TIMP1 | 9.14E−25 | 0.757438797 | 0.472 | 0.358 |
| IGHA2 | 9.34E−25 | 1.328805403 | 0.218 | 0.038 |
| RGS5 | 1.06E−24 | 0.998525325 | 0.436 | 0.188 |
| WFDC2 | 1.20E−24 | −1.176791641 | 0.076 | 0.26 |
| EFEMP1 | 4.37E−24 | 0.954054765 | 0.319 | 0.121 |
| PAPPA | 7.30E−24 | 0.966657001 | 0.226 | 0.048 |
| LDHA | 1.20E−23 | 0.752178805 | 0.416 | 0.241 |
| LAMB1 | 3.08E−23 | −0.660119075 | 0.263 | 0.512 |
| KCNE4 | 3.55E−23 | 0.891183144 | 0.414 | 0.194 |
| ITGB1 | 6.10E−23 | 0.593962472 | 0.507 | 0.388 |
| CTGF | 3.01E−22 | 1.114684447 | 0.356 | 0.174 |
| DUSP1 | 3.71E−22 | 0.588147362 | 0.686 | 0.51 |
| ATP1A2 | 8.88E−22 | −0.744150449 | 0.044 | 0.203 |
| COL8A1 | 1.49E−21 | 0.828101565 | 0.386 | 0.212 |
| TPM2 | 2.77E−21 | 0.668742031 | 0.534 | 0.371 |
| COL6A2 | 9.46E−21 | 0.697987723 | 0.438 | 0.307 |
| CSRP2 | 1.36E−20 | −0.622163154 | 0.237 | 0.473 |
| RGS4 | 2.94E−20 | 1.032603817 | 0.257 | 0.084 |
| SLC12A2 | 4.34E−20 | −0.790053512 | 0.068 | 0.236 |
| SLC39A6 | 5.46E−20 | 0.747221739 | 0.382 | 0.229 |
| TPM4 | 1.37E−19 | 0.570947387 | 0.507 | 0.4 |
| MTRNR2L5 | 1.65E−19 | −0.785684926 | 0.104 | 0.285 |
| DDX17 | 1.87E−19 | −0.471934911 | 0.344 | 0.585 |
| CCDC3 | 3.31E−19 | −0.476901609 | 0.123 | 0.305 |
| FMO3 | 3.56E−19 | −0.384294412 | 0.104 | 0.263 |
| NEAT1 | 6.25E−19 | −0.446545389 | 0.193 | 0.397 |
| RAD21 | 6.34E−19 | 0.615366637 | 0.353 | 0.26 |
| SELM | 1.60E−18 | 0.448103471 | 0.618 | 0.558 |
| METTL7A | 4.64E−18 | −0.422570824 | 0.16 | 0.335 |
| FSTL1 | 4.91E−18 | 0.594474039 | 0.449 | 0.329 |
| MT-ATP6 | 5.56E−18 | −0.49083842 | 0.301 | 0.528 |
| CRABP1 | 6.42E−18 | 0.9478779 | 0.257 | 0.115 |
| IGF2 | 1.02E−17 | 0.753313613 | 0.396 | 0.227 |
| ADIRF | 1.99E−17 | −0.500019052 | 0.217 | 0.426 |
| AGR2 | 2.33E−17 | −0.85278266 | 0.078 | 0.239 |
| NBL1 | 4.04E−17 | −0.407278541 | 0.332 | 0.548 |
| MT-CO2 | 5.44E−17 | −0.498495012 | 0.374 | 0.598 |
| SLC7A2 | 5.69E−17 | −0.579154083 | 0.095 | 0.254 |
| NFIA | 1.05E−16 | −0.621251249 | 0.077 | 0.234 |
| CCL2 | 1.14E−16 | 0.681692761 | 0.438 | 0.256 |
| MT-CO1 | 1.65E−16 | −0.268941719 | 0.462 | 0.678 |
| MTRNR2L8 | 1.97E−16 | −0.457524119 | 0.718 | 0.841 |
| COL6A1 | 2.03E−16 | 0.659960091 | 0.42 | 0.282 |
| RNA18S5 | 3.38E−16 | −0.648597258 | 0.082 | 0.223 |
| GPX3 | 4.39E−16 | 0.701833012 | 0.334 | 0.165 |
| CST3 | 5.09E−16 | −0.466241374 | 0.317 | 0.528 |
| TMEM176B | 5.16E−16 | 0.656650859 | 0.352 | 0.223 |
| MT-ND5 | 9.03E−16 | −0.461214217 | 0.34 | 0.558 |
| ID3 | 9.20E−16 | 0.659081967 | 0.458 | 0.342 |
| ALDH2 | 1.40E−15 | −0.402806205 | 0.092 | 0.232 |
| LAMP5 | 1.60E−15 | 0.607797435 | 0.432 | 0.247 |
| OAZ1 | 1.78E−15 | 0.441904405 | 0.482 | 0.408 |
| SFRP2 | 2.55E−15 | −0.546596639 | 0.506 | 0.676 |
| CXCL2 | 4.18E−15 | 0.922082662 | 0.299 | 0.159 |
| CCL11 | 6.01E−15 | 1.137909076 | 0.214 | 0.086 |
| TPI1 | 2.83E−14 | 0.574182349 | 0.364 | 0.261 |
| GAPDH | 5.55E−14 | 0.382308108 | 0.545 | 0.486 |
| PIK3R1 | 6.68E−14 | −0.471781138 | 0.393 | 0.585 |
| PTN | 1.62E−13 | −0.592200294 | 0.224 | 0.399 |
| SULF1 | 2.49E−13 | 0.677856898 | 0.325 | 0.165 |
| CRABP2 | 3.38E−13 | 0.463632418 | 0.492 | 0.41 |
| AQP3 | 3.57E−13 | −0.803289648 | 0.082 | 0.205 |
| LUC7L3 | 3.77E−13 | −0.516447881 | 0.156 | 0.324 |
| PLOD2 | 4.00E−13 | 0.706437645 | 0.205 | 0.075 |
| GNG11 | 4.76E−13 | 0.592680022 | 0.379 | 0.249 |
| VPS13C | 5.86E−13 | −0.457476131 | 0.121 | 0.272 |
| OST4 | 9.42E−13 | 0.418486371 | 0.424 | 0.356 |
| ACTB | 9.49E−13 | 0.392231333 | 0.574 | 0.517 |
| CXCL14 | 1.05E−12 | 0.537715636 | 0.486 | 0.34 |
| RPL5 | 1.31E−12 | −0.291562262 | 0.709 | 0.856 |
| ZYX | 1.61E−12 | 0.646759665 | 0.232 | 0.093 |
| PKM | 1.64E−12 | 0.42256687 | 0.347 | 0.291 |
| CPXM2 | 2.60E−12 | 0.631272464 | 0.252 | 0.117 |
| RBM25 | 2.81E−12 | −0.276738909 | 0.169 | 0.307 |
| RPS4X | 2.83E−12 | −0.26498687 | 0.789 | 0.901 |
| MT-ND4L | 4.18E−12 | −0.473837945 | 0.154 | 0.313 |
| CTSL | 4.43E−12 | 0.617859164 | 0.278 | 0.179 |
| S100A11 | 4.72E−12 | 0.381895177 | 0.499 | 0.435 |
| AHNAK | 6.17E−12 | −0.311526478 | 0.391 | 0.574 |
| IFITM1 | 7.21E−12 | 0.494975273 | 0.399 | 0.309 |
| RPS21 | 8.75E−12 | −0.291954829 | 0.801 | 0.892 |
| TNFSF10 | 1.04E−11 | −0.410752412 | 0.115 | 0.252 |
| TMEM100 | 1.17E−11 | −0.550083351 | 0.112 | 0.247 |
| ADCY3 | 1.56E−11 | 0.631430455 | 0.208 | 0.082 |
| GPR124 | 1.75E−11 | −0.389820559 | 0.108 | 0.229 |
| HIF1A | 2.44E−11 | 0.627327438 | 0.268 | 0.141 |
| CLK1 | 2.78E−11 | −0.277902484 | 0.155 | 0.287 |
| EGR1 | 3.14E−11 | 0.361295005 | 0.678 | 0.603 |
| PNISR | 3.16E−11 | −0.471172134 | 0.139 | 0.283 |
| IGFBP3 | 3.40E−11 | 0.770650408 | 0.236 | 0.146 |
| SNHG8 | 3.79E−11 | −0.395703475 | 0.198 | 0.36 |
| GAS5 | 4.88E−11 | −0.332986394 | 0.398 | 0.581 |
| TAGLN2 | 5.73E−11 | 0.498185095 | 0.26 | 0.19 |
| THY1 | 6.50E−11 | 0.482610922 | 0.496 | 0.362 |
| JMJD1C | 6.67E−11 | −0.405127042 | 0.099 | 0.219 |
| MIF | 7.98E−11 | 0.545164936 | 0.328 | 0.207 |
| CYR61 | 8.03E−11 | 0.660575398 | 0.341 | 0.227 |
| UBBP4 | 1.03E−10 | 0.601687194 | 0.251 | 0.135 |
| SIK1 | 1.12E−10 | 0.646630207 | 0.21 | 0.104 |
| ALCAM | 1.24E−10 | −0.522335755 | 0.088 | 0.212 |
| ARRDC3 | 1.26E−10 | 0.64083844 | 0.263 | 0.15 |
| COL1A1 | 1.46E−10 | 0.461951138 | 0.574 | 0.486 |
| SPTBN1 | 1.55E−10 | −0.419557537 | 0.11 | 0.238 |
| AKAP9 | 2.46E−10 | −0.305451313 | 0.189 | 0.316 |
| IER2 | 2.47E−10 | 0.441240221 | 0.456 | 0.371 |
| EPB41L4A-AS1 | 2.61E−10 | −0.312440774 | 0.106 | 0.221 |
| GPNMB | 4.45E−10 | −0.361573072 | 0.161 | 0.296 |
| LMO7 | 5.89E−10 | 0.486213023 | 0.236 | 0.172 |
| CLU | 6.30E−10 | −0.391730637 | 0.494 | 0.64 |
| PERP | 7.12E−10 | −0.533916048 | 0.124 | 0.254 |
| EPAS1 | 1.44E−09 | −0.491239774 | 0.123 | 0.252 |
| SRSF5 | 1.44E−09 | −0.257141321 | 0.302 | 0.455 |
| HES1 | 1.50E−09 | −0.491956051 | 0.165 | 0.307 |
| ARHGAP5 | 1.83E−09 | −0.260755233 | 0.125 | 0.236 |
| CEBPD | 2.04E−09 | 0.471019972 | 0.306 | 0.236 |
| PDLIM2 | 2.17E−09 | 0.493860139 | 0.203 | 0.119 |
| RGS16 | 2.67E−09 | 0.66254129 | 0.239 | 0.152 |
| ENO1 | 2.73E−09 | 0.38345859 | 0.347 | 0.293 |
| COL16A1 | 3.49E−09 | 0.588322687 | 0.206 | 0.101 |
| TSC22D1 | 4.10E−09 | −0.405879086 | 0.214 | 0.364 |
| ASH1L | 4.22E−09 | −0.335599263 | 0.115 | 0.232 |
| EIF4B | 4.40E−09 | −0.304118332 | 0.172 | 0.296 |
| KRT19 | 5.55E−09 | −0.637953559 | 0.192 | 0.3 |
| PHIP | 7.11E−09 | −0.29634243 | 0.104 | 0.208 |
| JUN | 9.26E−09 | 0.325677458 | 0.766 | 0.662 |
| MRPS6 | 1.24E−08 | 0.493096035 | 0.282 | 0.199 |
| SNRNP70 | 1.72E−08 | −0.385861975 | 0.122 | 0.234 |
| PCM1 | 1.82E−08 | −0.299728384 | 0.176 | 0.303 |
| COL5A2 | 2.37E−08 | 0.543285554 | 0.222 | 0.11 |
| HCFC1R1 | 2.67E−08 | 0.464201766 | 0.234 | 0.152 |
| PEA15 | 3.38E−08 | 0.489186632 | 0.227 | 0.135 |
| FBN1 | 3.87E−08 | 0.519278837 | 0.246 | 0.137 |
| POLR2L | 3.90E−08 | 0.358267095 | 0.405 | 0.34 |
| SESN3 | 4.78E−08 | −0.327994144 | 0.141 | 0.252 |
| SYNE1 | 4.81E−08 | −0.302908587 | 0.11 | 0.207 |
| ARPC5 | 4.93E−08 | 0.414633707 | 0.299 | 0.245 |
| PDGFRA | 5.06E−08 | 0.345105569 | 0.409 | 0.356 |
| EZR | 6.27E−08 | −0.355279936 | 0.125 | 0.239 |
| VCAM1 | 7.25E−08 | 0.488976998 | 0.233 | 0.13 |
| EDNRA | 7.63E−08 | −0.308667569 | 0.147 | 0.252 |
| OSTC | 7.85E−08 | 0.445148793 | 0.304 | 0.219 |
| A2M | 8.53E−08 | −0.360831698 | 0.256 | 0.397 |
| GSN | 9.06E−08 | −0.270771129 | 0.268 | 0.408 |
| PTRF | 1.41E−07 | 0.353626925 | 0.418 | 0.36 |
| PPDPF | 1.42E−07 | −0.288236076 | 0.19 | 0.314 |
| CTNNAL1 | 1.56E−07 | 0.480876008 | 0.246 | 0.159 |
| ARPC2 | 1.59E−07 | 0.389404653 | 0.324 | 0.263 |
| PPIB | 2.75E−07 | 0.347790912 | 0.405 | 0.347 |
| SNHG6 | 3.09E−07 | −0.289509866 | 0.26 | 0.391 |
| ZHX1 | 3.17E−07 | −0.373048262 | 0.154 | 0.25 |
| TAGLN | 4.66E−07 | 0.386758717 | 0.54 | 0.453 |
| APOE | 6.90E−07 | −0.510672756 | 0.161 | 0.276 |
| FXYD3 | 7.30E−07 | −0.277899295 | 0.115 | 0.212 |
| TMEM176A | 8.30E−07 | 0.455615034 | 0.272 | 0.172 |
| FGF7 | 1.10E−06 | 0.385686265 | 0.456 | 0.375 |
| ACTN1 | 1.60E−06 | 0.432476791 | 0.264 | 0.179 |
| MAFB | 1.65E−06 | −0.37283683 | 0.123 | 0.227 |
| TNC | 2.10E−06 | 0.484760564 | 0.257 | 0.166 |
| SERPING1 | 2.37E−06 | −0.251084541 | 0.511 | 0.645 |
| SLC6A6 | 2.85E−06 | 0.392693515 | 0.267 | 0.214 |
| NFKBIZ | 3.00E−06 | 0.459027717 | 0.212 | 0.141 |
| MMP2 | 3.32E−06 | 0.394417967 | 0.243 | 0.183 |
| SPTSSA | 4.27E−06 | 0.423139342 | 0.218 | 0.15 |
| RPL7 | 5.04E−06 | 0.406415074 | 0.221 | 0.148 |
| UTRN | 5.77E−06 | −0.272327357 | 0.135 | 0.227 |
| PRSS23 | 8.22E−06 | 0.390301992 | 0.346 | 0.274 |
| GLIPR1 | 1.96E−05 | 0.356234427 | 0.399 | 0.303 |
| MYL9 | 2.18E−05 | 0.304252465 | 0.404 | 0.335 |
| NDUFB2 | 2.31E−05 | 0.323582218 | 0.351 | 0.293 |
| ADAMTS1 | 2.99E−05 | 0.446277742 | 0.309 | 0.227 |
| CALU | 3.06E−05 | 0.372862131 | 0.257 | 0.176 |
| C3orf58 | 3.20E−05 | 0.369694142 | 0.304 | 0.245 |
| PREX2 | 3.43E−05 | −0.315497969 | 0.115 | 0.201 |
| ALDH1A2 | 5.96E−05 | −0.328312722 | 0.141 | 0.232 |
| IGFBP6 | 6.11E−05 | −0.271984755 | 0.425 | 0.543 |
| VCAN | 6.87E−05 | 0.294268565 | 0.472 | 0.389 |
| EDIL3 | 8.51E−05 | 0.344248059 | 0.303 | 0.23 |
| TUBA1A | 9.37E−05 | 0.377031009 | 0.322 | 0.25 |
| EPHB6 | 0.000149103 | 0.375908229 | 0.245 | 0.155 |
| SRSF11 | 0.000202615 | −0.250228806 | 0.221 | 0.32 |
| TFAP2A | 0.00028137 | −0.265572372 | 0.189 | 0.28 |
| ABI3BP | 0.000349964 | −0.262134598 | 0.135 | 0.216 |
| NONO | 0.000383839 | −0.263358226 | 0.13 | 0.21 |
| CSTB | 0.000398494 | −0.251803999 | 0.149 | 0.23 |
| AP2M1 | 0.000453169 | 0.292522217 | 0.245 | 0.192 |
| IFI6 | 0.000540467 | −0.305488454 | 0.159 | 0.243 |
| AKAP12 | 0.000778343 | −0.277970326 | 0.176 | 0.258 |
| CPXM1 | 0.000866838 | −0.262913574 | 0.261 | 0.356 |
| F3 | 0.009212926 | −0.266898185 | 0.176 | 0.247 |
| RAN | 0.01213486 | 0.25073803 | 0.251 | 0.194 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 7.

TABLE 7

| | p_val | avg_diff | pct. 1 | pct. 2 |
|---|---|---|---|---|
| STATH | 7.18E−97 | −3.001485389 | 0.031 | 0.859 |
| BPIFA1 | 2.02E−46 | −1.842972495 | 0.132 | 0.756 |
| ZG16B | 1.10E−27 | −1.226017378 | 0.024 | 0.363 |
| HLA-DQA2 | 5.12E−24 | −1.219100808 | 0.188 | 0.615 |
| F13A1 | 6.96E−20 | 1.727958681 | 0.488 | 0.156 |
| IGJ | 2.77E−19 | 1.503968413 | 0.632 | 0.237 |
| SLPI | 7.69E−16 | −1.049129314 | 0.294 | 0.674 |
| BPIFB1 | 1.17E−15 | −0.821279482 | 0.124 | 0.452 |
| IGHA1 | 2.63E−15 | 1.025115049 | 0.658 | 0.341 |
| MSMB | 9.70E−14 | −0.695527649 | 0.024 | 0.222 |
| CXCR4 | 2.65E−12 | −0.846536769 | 0.331 | 0.637 |
| TIMP1 | 2.16E−11 | 1.181837303 | 0.481 | 0.274 |
| WFDC2 | 2.58E−11 | −0.851391428 | 0.093 | 0.348 |
| IGHG4 | 1.04E−10 | 2.125698557 | 0.291 | 0.059 |
| PLAC8 | 1.10E−10 | −0.687752501 | 0.055 | 0.259 |
| CTSC | 2.20E−10 | 0.89249982 | 0.537 | 0.437 |
| RPL30 | 4.76E−10 | −0.44180483 | 0.883 | 0.97 |
| PIGR | 8.56E−10 | −0.712860007 | 0.046 | 0.237 |
| RNASE1 | 3.92E−09 | 1.970180435 | 0.216 | 0.059 |
| RPS27A | 8.58E−09 | −0.32924168 | 0.848 | 0.985 |
| SLC2A3 | 9.48E−09 | 1.005097238 | 0.34 | 0.148 |
| RPS3 | 1.19E−08 | −0.347628181 | 0.72 | 0.933 |
| RPL26 | 1.54E−08 | −0.310964868 | 0.76 | 0.956 |
| MT-ATP6 | 2.38E−08 | 0.279038574 | 0.303 | 0.385 |
| CD52 | 3.17E−08 | 1.020173721 | 0.469 | 0.237 |
| RPL19 | 8.24E−08 | −0.342063138 | 0.871 | 0.963 |
| RGS1 | 1.17E−07 | −0.692068372 | 0.51 | 0.726 |
| TOMM7 | 1.65E−07 | −0.373475989 | 0.32 | 0.57 |
| CORO1A | 2.04E−07 | −0.330944051 | 0.217 | 0.437 |
| SH3BGRL3 | 2.13E−07 | 0.590810079 | 0.651 | 0.511 |
| RPS12 | 2.47E−07 | −0.279384689 | 0.92 | 0.993 |
| SAMSN1 | 4.04E−07 | 0.963597968 | 0.487 | 0.311 |
| HLA-DPA1 | 4.17E−07 | −0.402352659 | 0.848 | 0.963 |
| CST3 | 5.11E−07 | −0.277456851 | 0.82 | 0.978 |
| RPL37 | 6.35E−07 | −0.311119935 | 0.744 | 0.933 |
| RPL27 | 6.87E−07 | −0.322277849 | 0.902 | 0.985 |
| ERP29 | 1.10E−06 | −0.266426 | 0.133 | 0.296 |
| CTSB | 1.23E−06 | 0.460644222 | 0.675 | 0.741 |
| HLA-DQB2 | 1.64E−06 | −0.466723367 | 0.178 | 0.393 |
| RPL18 | 1.79E−06 | −0.267949549 | 0.587 | 0.822 |
| RPS23 | 1.90E−06 | −0.357721603 | 0.821 | 0.941 |
| RPS21 | 2.33E−06 | −0.3174624 | 0.867 | 0.97 |
| THBS1 | 2.52E−06 | 1.088448915 | 0.32 | 0.156 |
| OGFRL1 | 2.84E−06 | −0.666509209 | 0.132 | 0.326 |
| RPS4X | 3.85E−06 | −0.285585663 | 0.747 | 0.926 |
| IFI30 | 3.89E−06 | 0.252282295 | 0.71 | 0.763 |
| HBEGF | 4.08E−06 | 0.722502506 | 0.26 | 0.207 |
| ITM2B | 4.46E−06 | −0.409751252 | 0.602 | 0.815 |
| RPL23A | 5.41E−06 | −0.278364159 | 0.553 | 0.785 |
| AREG | 5.67E−06 | 0.80185493 | 0.377 | 0.252 |
| MPEG1 | 6.61E−06 | −0.467720313 | 0.172 | 0.378 |
| NAP1L1 | 8.06E−06 | −0.477018502 | 0.322 | 0.556 |
| CLEC2B | 8.57E−06 | −0.315441415 | 0.247 | 0.452 |
| IGHA2 | 1.06E−05 | 1.176519931 | 0.269 | 0.089 |
| FAM26F | 1.10E−05 | −0.529020196 | 0.262 | 0.452 |
| ALCAM | 1.10E−05 | −0.466308621 | 0.092 | 0.252 |
| TNFSF10 | 1.16E−05 | −0.514600496 | 0.077 | 0.23 |
| CCDC50 | 1.16E−05 | −0.383349808 | 0.096 | 0.252 |
| DUSP2 | 1.22E−05 | 0.524870861 | 0.689 | 0.519 |
| SGK1 | 1.27E−05 | −0.523333633 | 0.414 | 0.63 |
| ALOX15 | 2.05E−05 | 0.891994435 | 0.269 | 0.104 |
| ZFP36L2 | 2.09E−05 | −0.425638703 | 0.321 | 0.548 |
| SETD2 | 2.19E−05 | −0.451213831 | 0.071 | 0.215 |
| G0S2 | 2.21E−05 | 1.048568477 | 0.312 | 0.133 |
| NDFIP1 | 2.28E−05 | −0.51413316 | 0.124 | 0.304 |
| HINT1 | 2.59E−05 | −0.269107 | 0.373 | 0.585 |
| CCNI | 2.63E−05 | −0.278778841 | 0.265 | 0.459 |
| LYN | 2.76E−05 | −0.40511994 | 0.123 | 0.289 |
| HLA-DPB1 | 3.04E−05 | −0.306004579 | 0.843 | 0.956 |
| ADAM28 | 3.09E−05 | −0.512283443 | 0.072 | 0.215 |
| WSB1 | 3.39E−05 | −0.346136626 | 0.251 | 0.452 |
| KRT19 | 3.57E−05 | −0.744412855 | 0.151 | 0.311 |
| NFKBIA | 4.03E−05 | 0.396633316 | 0.831 | 0.733 |
| TSC22D3 | 4.50E−05 | −0.295735279 | 0.499 | 0.719 |
| RPS13 | 4.73E−05 | −0.268201386 | 0.882 | 0.941 |
| STAT1 | 5.33E−05 | −0.392411738 | 0.112 | 0.267 |
| CLEC10A | 5.61E−05 | 0.55570363 | 0.466 | 0.326 |
| MIF | 6.16E−05 | 0.504668379 | 0.253 | 0.178 |
| CD163 | 7.37E−05 | 0.82102558 | 0.302 | 0.148 |
| XIST | 8.74E−05 | −0.545477768 | 0.167 | 0.348 |
| POLR1D | 8.77E−05 | −0.308305436 | 0.12 | 0.267 |
| RPL17 | 9.20E−05 | 0.560510182 | 0.274 | 0.215 |
| MARCH1 | 9.30E−05 | −0.29110411 | 0.173 | 0.333 |
| FCER1A | 9.96E−05 | −0.663602485 | 0.266 | 0.437 |
| SNHG8 | 0.000107528 | −0.339954335 | 0.234 | 0.422 |
| EREG | 0.000116215 | 1.04971632 | 0.26 | 0.119 |
| IFI6 | 0.000134392 | −0.501871692 | 0.071 | 0.207 |
| UXT | 0.000135162 | −0.271863372 | 0.108 | 0.237 |
| CTSL | 0.000164391 | 0.817679901 | 0.21 | 0.119 |
| SMIM14 | 0.000172009 | −0.263439712 | 0.096 | 0.222 |
| P2RY13 | 0.000181472 | −0.626825591 | 0.107 | 0.244 |
| MTRNR2L1 | 0.000183529 | 0.305706582 | 0.851 | 0.793 |
| RPL35A | 0.000184342 | −0.250400473 | 0.876 | 0.97 |
| LIPA | 0.000213015 | 0.620678941 | 0.33 | 0.207 |
| FPR1 | 0.000257803 | 0.783765469 | 0.243 | 0.104 |
| CASP1 | 0.0002609 | −0.363623878 | 0.098 | 0.23 |
| MAFB | 0.000318882 | 0.264683028 | 0.32 | 0.378 |
| PTRHD1 | 0.00035999 | −0.282379933 | 0.126 | 0.259 |
| SLC31A2 | 0.000393934 | −0.319058983 | 0.151 | 0.281 |
| HIGD2A | 0.000415118 | −0.259431362 | 0.2 | 0.356 |
| FBP1 | 0.000416378 | 0.488984212 | 0.219 | 0.163 |
| RBPJ | 0.00056734 | 0.552223473 | 0.305 | 0.23 |
| TMED9 | 0.000573861 | 0.51639245 | 0.226 | 0.163 |
| VCAN | 0.000591167 | 0.728204722 | 0.263 | 0.133 |
| ZNF331 | 0.000625871 | 0.523501408 | 0.493 | 0.37 |
| PLAUR | 0.000633161 | 0.450452664 | 0.646 | 0.57 |
| CXCL16 | 0.000650393 | −0.442364087 | 0.209 | 0.378 |
| BLVRB | 0.000671271 | 0.509512575 | 0.247 | 0.185 |
| CYTIP | 0.000683841 | −0.514464342 | 0.186 | 0.348 |
| POSTN | 0.000737545 | 0.892155587 | 0.249 | 0.104 |
| S100A9 | 0.000928806 | 0.494153614 | 0.407 | 0.237 |
| MTRNR2L6 | 0.001173947 | −0.325169704 | 0.109 | 0.23 |
| EIF3H | 0.001652068 | −0.250242402 | 0.24 | 0.385 |
| LY96 | 0.001709557 | 0.482789024 | 0.278 | 0.178 |
| MYL6 | 0.001799005 | 0.300985492 | 0.825 | 0.733 |
| CEBPD | 0.001814517 | −0.362449169 | 0.235 | 0.4 |
| MS4A4A | 0.002040244 | 0.70656183 | 0.201 | 0.089 |
| KLF4 | 0.002184751 | −0.39345667 | 0.327 | 0.496 |
| ZFAND5 | 0.002276671 | −0.362930063 | 0.402 | 0.563 |
| TPM4 | 0.002289765 | 0.417023794 | 0.432 | 0.356 |
| NAGK | 0.002524115 | −0.282782237 | 0.141 | 0.267 |
| EMB | 0.002651311 | 0.501867985 | 0.25 | 0.178 |
| MIS18BP1 | 0.002680015 | −0.325259062 | 0.161 | 0.296 |
| PARP14 | 0.002909262 | −0.323457371 | 0.111 | 0.23 |
| FBL | 0.002933871 | −0.301183562 | 0.133 | 0.259 |
| SAT1 | 0.002994187 | −0.273437183 | 0.735 | 0.852 |
| RPS2 | 0.003185341 | −0.281746689 | 0.59 | 0.748 |
| CSTB | 0.003689014 | 0.517931992 | 0.275 | 0.193 |
| ITGA4 | 0.003977735 | −0.297546398 | 0.167 | 0.296 |
| PDIA6 | 0.004062177 | 0.490551332 | 0.331 | 0.237 |
| MALAT1 | 0.004249267 | −0.395787519 | 0.877 | 0.933 |
| IRF8 | 0.004629778 | −0.264589382 | 0.164 | 0.281 |
| CSTA | 0.004819432 | −0.366465373 | 0.2 | 0.341 |
| PRDX6 | 0.004893835 | −0.422528824 | 0.148 | 0.281 |
| YTHDC1 | 0.005447029 | −0.295989717 | 0.111 | 0.222 |
| ACTR3 | 0.005570457 | 0.37219172 | 0.389 | 0.333 |
| RGCC | 0.005614531 | 0.486794582 | 0.293 | 0.193 |
| NAMPTL | 0.00632544 | 0.494645211 | 0.281 | 0.2 |
| HES1 | 0.006602326 | −0.561086654 | 0.109 | 0.222 |
| LMNA | 0.007162332 | 0.486645721 | 0.299 | 0.23 |
| AIM1 | 0.008161951 | −0.318825822 | 0.101 | 0.207 |
| MTRNR2L11 | 0.008452979 | −0.353512485 | 0.109 | 0.222 |
| S100A8 | 0.010257191 | 0.841434007 | 0.268 | 0.141 |
| RNH1 | 0.010699273 | 0.424226464 | 0.271 | 0.193 |
| VTRNA1-1 | 0.010720755 | −0.36784839 | 0.21 | 0.348 |
| FCGR3A | 0.012312956 | −0.549403284 | 0.161 | 0.244 |
| CARD16 | 0.01286838 | −0.313228487 | 0.109 | 0.215 |
| TES | 0.014051552 | −0.251728506 | 0.126 | 0.222 |
| CD1C | 0.014788592 | 0.621956828 | 0.24 | 0.148 |
| A2M | 0.015506218 | −0.501526332 | 0.105 | 0.207 |
| CDC37 | 0.016064216 | 0.447689734 | 0.21 | 0.141 |
| G3BP1 | 0.016572569 | −0.26010389 | 0.138 | 0.244 |
| EVI2B | 0.016973758 | −0.305102866 | 0.244 | 0.378 |
| TMEM14C | 0.017820432 | −0.280966265 | 0.158 | 0.274 |
| DUSP6 | 0.020868489 | −0.282832722 | 0.197 | 0.319 |
| MXD1 | 0.028383237 | 0.541399195 | 0.231 | 0.148 |
| CXCL2 | 0.03294536 | 0.39708324 | 0.453 | 0.393 |
| HIST1H4C | 0.035801724 | −0.394560078 | 0.136 | 0.23 |
| EIF3F | 0.043877918 | −0.288269557 | 0.21 | 0.326 |
| TXNDC17 | 0.046552496 | 0.30785666 | 0.234 | 0.178 |
| LAIR1 | 0.049236548 | 0.371759143 | 0.207 | 0.156 |
| PET100 | 0.05322369 | 0.37666952 | 0.222 | 0.148 |
| RHOB | 0.061300128 | −0.291267468 | 0.138 | 0.23 |
| RGS18 | 0.071740633 | −0.31990829 | 0.132 | 0.222 |
| VMP1 | 0.079257494 | −0.360534144 | 0.175 | 0.259 |
| SMARCA5 | 0.084318153 | −0.309509153 | 0.194 | 0.296 |
| HMGB2 | 0.110905412 | −0.369809446 | 0.17 | 0.259 |
| FCN1 | 0.139697685 | 0.369579938 | 0.311 | 0.237 |
| NLRP3 | 0.264639425 | −0.27708171 | 0.265 | 0.348 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 8.

TABLE 8

| | p_val | avg_diff | pct. 1 | pct. 2 |
|---|---|---|---|---|
| STATH | 1.61E−30 | −3.227551658 | 0.024 | 0.926 |
| MSMB | 1.10E−10 | −1.748741499 | 0.012 | 0.444 |
| LYZ | 4.20E−10 | −2.864081673 | 0.069 | 0.519 |
| BPIFA1 | 1.10E−09 | −1.246110242 | 0.134 | 0.741 |
| ZG16B | 8.69E−08 | −1.229585496 | 0.008 | 0.333 |
| CD52 | 9.78E−07 | 1.841038481 | 0.671 | 0.222 |
| IGJ | 3.16E−06 | 2.899666647 | 0.622 | 0.222 |
| IGHA1 | 3.35E−06 | 2.73590403 | 0.663 | 0.296 |
| MS4A2 | 3.80E−06 | 0.266526955 | 0.443 | 0.704 |
| LTF | 4.07E−06 | −1.216237779 | 0.012 | 0.296 |
| HPGD | 4.60E−06 | 1.101768541 | 0.854 | 0.778 |
| CHCHD1 | 6.80E−06 | −0.348684633 | 0.033 | 0.222 |
| PHKB | 6.83E−06 | −0.410543711 | 0.02 | 0.222 |
| LDHB | 7.70E−06 | −0.66641308 | 0.146 | 0.519 |
| SRSF9 | 8.32E−06 | −0.643105847 | 0.085 | 0.444 |
| RSBN1L | 8.36E−06 | −0.639142398 | 0.053 | 0.333 |
| WDR83OS | 1.09E−05 | −0.349124188 | 0.13 | 0.444 |
| SLPI | 1.29E−05 | −1.567005221 | 0.317 | 0.704 |
| GSTK1 | 1.39E−05 | −0.634362171 | 0.077 | 0.407 |
| PRDX5 | 1.66E−05 | −0.338423593 | 0.167 | 0.481 |
| NR4A1 | 1.66E−05 | 0.860236615 | 0.659 | 0.556 |
| AREG | 2.52E−05 | 1.904769964 | 0.524 | 0.074 |
| MT-CO3 | 2.54E−05 | 0.592545374 | 0.504 | 0.667 |
| BPIFB1 | 3.01E−05 | −0.996975067 | 0.114 | 0.519 |
| VIMP | 3.14E−05 | −0.448073429 | 0.114 | 0.407 |
| SAP18 | 3.68E−05 | −0.404911845 | 0.171 | 0.519 |
| NAP1L1 | 5.06E−05 | 0.594430293 | 0.35 | 0.444 |
| HNRNPR | 6.78E−05 | −0.528336591 | 0.098 | 0.37 |
| AGR2 | 6.97E−05 | −1.358659259 | 0.098 | 0.444 |
| CWF19L2 | 7.78E−05 | −0.647448714 | 0.033 | 0.259 |
| ATP6V1F | 8.51E−05 | −0.416646171 | 0.106 | 0.37 |
| EEF1B2 | 9.99E−05 | −0.610503285 | 0.386 | 0.815 |
| NDUFS1 | 0.000118007 | −0.617205333 | 0.045 | 0.296 |
| NDUFA2 | 0.000131839 | −0.656169714 | 0.069 | 0.37 |
| BRK1 | 0.000134723 | −0.570033097 | 0.118 | 0.444 |
| LMO4 | 0.000139303 | −0.881151611 | 0.293 | 0.704 |
| SEPPI | 0.000144728 | −0.576996792 | 0.045 | 0.296 |
| MLF2 | 0.000156196 | −0.741485056 | 0.065 | 0.37 |
| TRAPPC2L | 0.000165658 | −0.485846781 | 0.033 | 0.259 |
| PDCD4 | 0.000169933 | −0.252987952 | 0.159 | 0.37 |
| ANXA4 | 0.000170538 | −0.767608131 | 0.106 | 0.444 |
| IQGAP1 | 0.000213864 | −0.487278971 | 0.211 | 0.556 |
| HSPA5 | 0.000215158 | 1.263128277 | 0.508 | 0.296 |
| MRPL20 | 0.00021754 | −0.518581993 | 0.093 | 0.37 |
| FUS | 0.00022632 | −0.47442121 | 0.159 | 0.481 |
| POSTN | 0.000228735 | 1.782623378 | 0.28 | 0.111 |
| HIST1H4C | 0.000238909 | −0.756200985 | 0.134 | 0.481 |
| LSM7 | 0.00024123 | −0.649121471 | 0.114 | 0.444 |
| NDUFB2 | 0.000265564 | 0.485727095 | 0.358 | 0.444 |
| SRP14 | 0.0002666 | 0.388351179 | 0.325 | 0.444 |
| ATP5G2 | 0.000293869 | 0.263832646 | 0.305 | 0.444 |
| MT1E | 0.0002956 | −0.428529946 | 0.041 | 0.222 |
| TSPO | 0.000303163 | −0.565926622 | 0.24 | 0.63 |
| CAPN13 | 0.000308731 | −0.543190818 | 0.02 | 0.222 |
| XRCC5 | 0.000313788 | −0.437972849 | 0.183 | 0.519 |
| LMO7 | 0.00031797 | −0.481048243 | 0.02 | 0.222 |
| LAMP1 | 0.000327629 | −0.767428887 | 0.041 | 0.296 |
| BTK | 0.00036189 | −0.378113498 | 0.26 | 0.556 |
| ST13 | 0.000369538 | −0.461377198 | 0.098 | 0.37 |
| APEX1 | 0.000371653 | −0.6231601 | 0.081 | 0.37 |
| ACTB | 0.000403786 | 0.275674135 | 0.634 | 0.815 |
| PTPRF | 0.000412819 | −0.390040084 | 0.028 | 0.222 |
| ATP5A1 | 0.000453707 | −0.493146882 | 0.187 | 0.481 |
| EIF3L | 0.000457515 | −0.626094242 | 0.154 | 0.481 |
| WFDC2 | 0.000484226 | −1.152242302 | 0.102 | 0.444 |
| MYO9A | 0.000500818 | −0.732609609 | 0.028 | 0.222 |
| POLR1D | 0.000508782 | −0.397594572 | 0.102 | 0.37 |
| ATP5I | 0.000519003 | 0.263468249 | 0.248 | 0.37 |
| IGHG4 | 0.000539274 | 3.250109192 | 0.293 | 0 |
| PRKAR1A | 0.000597546 | −0.50851708 | 0.187 | 0.481 |
| EIF2S3 | 0.00061655 | 0.476167995 | 0.159 | 0.222 |
| EAPP | 0.000630452 | −0.590190376 | 0.041 | 0.222 |
| PUS7L | 0.000646388 | −0.917747662 | 0.02 | 0.222 |
| CLN5 | 0.000648076 | −0.49613207 | 0.041 | 0.259 |
| NR4A2 | 0.00066632 | 1.097021482 | 0.598 | 0.37 |
| NAPA | 0.000749858 | −0.480360633 | 0.049 | 0.222 |
| PHF3 | 0.00074992 | −0.286724448 | 0.122 | 0.37 |
| SERP1 | 0.000760293 | 0.289854349 | 0.159 | 0.259 |
| C4orf3 | 0.000796732 | 0.28521376 | 0.175 | 0.259 |
| MZT2B | 0.000812774 | −0.582220635 | 0.02 | 0.222 |
| DHX36 | 0.000840542 | −0.566435607 | 0.073 | 0.333 |
| NDRG2 | 0.000904331 | −0.380763465 | 0.069 | 0.296 |
| DUSP22 | 0.000908006 | −0.814282735 | 0.028 | 0.259 |
| MTDH | 0.000921371 | −0.277810051 | 0.232 | 0.481 |
| MPHOSPH8 | 0.000948324 | −0.599170104 | 0.057 | 0.296 |
| NDUFA11 | 0.000961167 | 0.773890245 | 0.24 | 0.185 |
| RBMX | 0.001009614 | −0.337078956 | 0.232 | 0.481 |
| C9orf78 | 0.001012171 | −0.432742244 | 0.098 | 0.259 |
| PSMD2 | 0.001019295 | −0.633883949 | 0.053 | 0.259 |
| NFAT5 | 0.001034279 | −0.463893249 | 0.045 | 0.259 |
| ARF1 | 0.00112234 | 0.265652023 | 0.187 | 0.296 |
| HEBP2 | 0.001145393 | −0.639976315 | 0.077 | 0.333 |
| NENF | 0.001190116 | −0.629396481 | 0.037 | 0.222 |
| GSN | 0.001281889 | −0.437480216 | 0.126 | 0.407 |
| IRF2 | 0.001322372 | −0.348124325 | 0.045 | 0.222 |
| ACSL4 | 0.001324575 | 0.799068408 | 0.65 | 0.556 |
| PIGR | 0.001344792 | −0.821278668 | 0.049 | 0.296 |
| MRPL33 | 0.001349417 | 0.258729588 | 0.215 | 0.37 |
| PSMB8 | 0.001358357 | −0.560468071 | 0.093 | 0.333 |
| NIPSNAP1 | 0.001362034 | −0.703047089 | 0.024 | 0.222 |
| MGST1 | 0.001367757 | −0.531404728 | 0.028 | 0.222 |
| ARL8B | 0.001438162 | −0.356769112 | 0.065 | 0.259 |
| BTG2 | 0.001481902 | 0.694805398 | 0.699 | 0.593 |
| MYOF | 0.001486835 | −0.499461953 | 0.045 | 0.259 |
| JAK1 | 0.001487558 | −0.32139568 | 0.102 | 0.296 |
| EMC10 | 0.001540089 | −0.303361294 | 0.085 | 0.296 |
| RAB1B | 0.001570213 | −0.538386049 | 0.045 | 0.259 |
| IER3 | 0.001602673 | 0.405002966 | 0.228 | 0.296 |
| HAVCR2 | 0.001680336 | −0.676837495 | 0.028 | 0.222 |
| EIF4G1 | 0.001692764 | −0.300622069 | 0.057 | 0.222 |
| COA3 | 0.001712207 | −0.590154401 | 0.041 | 0.259 |
| SCAMP1 | 0.00175368 | −0.628059087 | 0.024 | 0.222 |
| RPS2 | 0.001787959 | −0.45694103 | 0.402 | 0.778 |
| AURKAIP1 | 0.001807001 | −0.360977368 | 0.065 | 0.259 |
| CHD3 | 0.001855521 | −0.518777447 | 0.045 | 0.259 |
| ARPC1B | 0.001871043 | 0.26818281 | 0.22 | 0.333 |
| DCXR | 0.001907568 | −0.55626982 | 0.098 | 0.37 |
| MRPS36 | 0.001915418 | −0.499443422 | 0.061 | 0.296 |
| TUBB4B | 0.001918467 | −0.286680965 | 0.106 | 0.333 |
| PDIA6 | 0.001979825 | −0.492911919 | 0.215 | 0.519 |
| IDH2 | 0.002018831 | −0.425915021 | 0.041 | 0.222 |
| BIRC2 | 0.002026545 | −0.259370868 | 0.093 | 0.296 |
| TMEM259 | 0.002061883 | −0.839407888 | 0.02 | 0.222 |
| MGST3 | 0.002064945 | −0.567155102 | 0.057 | 0.259 |
| LSM3 | 0.002072851 | −0.725634985 | 0.085 | 0.333 |
| HNRNPA1 | 0.002093048 | −0.509182183 | 0.252 | 0.593 |
| UQCRC1 | 0.002139546 | −0.286889128 | 0.102 | 0.333 |
| NDUFS2 | 0.002187582 | −0.544021032 | 0.045 | 0.259 |
| IRF1 | 0.002225311 | 1.263407505 | 0.24 | 0.074 |
| SERPINB1 | 0.002364624 | 0.416623055 | 0.317 | 0.407 |
| NASP | 0.002386881 | −0.378247249 | 0.114 | 0.296 |
| MRPL4 | 0.002406948 | −0.50936705 | 0.045 | 0.259 |
| PLAT | 0.002408676 | −1.015294818 | 0.024 | 0.222 |
| COX7C | 0.002412442 | −0.369860493 | 0.439 | 0.778 |
| RHOH | 0.002501299 | −0.286594784 | 0.179 | 0.407 |
| SOX4 | 0.002517695 | −0.31256721 | 0.085 | 0.296 |
| NDUFB6 | 0.002547466 | −0.329738984 | 0.13 | 0.37 |
| TMEM176B | 0.002595892 | 0.935913882 | 0.252 | 0.148 |
| RPL21 | 0.002694332 | 0.378008798 | 0.236 | 0.296 |
| ADIRF | 0.002708643 | −0.577781161 | 0.085 | 0.333 |
| LIMA1 | 0.002712959 | −0.460536147 | 0.069 | 0.222 |
| COPS2 | 0.002806239 | −0.442443785 | 0.049 | 0.259 |
| TBC1D14 | 0.002836063 | −0.537028911 | 0.061 | 0.222 |
| TAF1D | 0.002837217 | −0.476765124 | 0.065 | 0.259 |
| SNX3 | 0.002856637 | −0.3917276 | 0.073 | 0.296 |
| OAT | 0.002899474 | −0.723886836 | 0.089 | 0.333 |
| RPS4X | 0.002973091 | −0.51759696 | 0.764 | 0.926 |
| IER2 | 0.003023207 | 0.291151901 | 0.581 | 0.741 |
| PSMD12 | 0.003041961 | −0.329552982 | 0.061 | 0.259 |
| MLPH | 0.003240217 | −0.647361249 | 0.041 | 0.222 |
| SF3B14 | 0.003247051 | 0.342729798 | 0.118 | 0.222 |
| TCEB2 | 0.003277482 | −0.265494225 | 0.256 | 0.481 |
| GOLGB1 | 0.003361151 | −0.44270083 | 0.098 | 0.333 |
| DDB1 | 0.003442347 | −0.507253274 | 0.089 | 0.333 |
| PCNA | 0.003453442 | −0.643065223 | 0.033 | 0.222 |
| KLF6 | 0.003526678 | 0.726557026 | 0.711 | 0.63 |
| MRPL16 | 0.00354099 | −0.320183122 | 0.053 | 0.222 |
| RPL32 | 0.003593817 | −0.259929319 | 0.768 | 1 |
| MT-ND3 | 0.003640544 | −0.579325028 | 0.394 | 0.741 |
| RPL34 | 0.0036583 | −0.27656674 | 0.923 | 1 |
| VDAC2 | 0.003662419 | −0.802275188 | 0.073 | 0.296 |
| NACA | 0.003677298 | −0.321522129 | 0.443 | 0.778 |
| EIF4B | 0.003808306 | −0.555538383 | 0.183 | 0.481 |
| BLOC1S1 | 0.003850881 | −0.337217375 | 0.081 | 0.259 |
| YWHAE | 0.003896269 | 0.325836027 | 0.215 | 0.296 |
| RPL37A | 0.003982024 | −0.343191175 | 0.793 | 1 |
| PTTG1IP | 0.004020909 | −0.62724566 | 0.13 | 0.407 |
| CUTA | 0.00403679 | −0.560547622 | 0.146 | 0.37 |
| SRSF11 | 0.004241107 | −0.482167034 | 0.154 | 0.333 |
| MTRNR2L11 | 0.004247818 | −0.574412729 | 0.171 | 0.37 |
| VMO1 | 0.004257642 | −0.291384307 | 0.081 | 0.259 |
| TLN1 | 0.004440976 | −0.273553932 | 0.22 | 0.444 |
| ORMDL1 | 0.004482901 | −0.476236446 | 0.073 | 0.296 |
| EHMT1 | 0.004594561 | −0.48793528 | 0.033 | 0.222 |
| RPL23A | 0.004951312 | −0.433453016 | 0.435 | 0.778 |
| UBL5 | 0.004963853 | −0.550965741 | 0.374 | 0.63 |
| H2AFV | 0.004969115 | −0.274891718 | 0.085 | 0.259 |
| MYH9 | 0.005057273 | 0.250674287 | 0.146 | 0.222 |
| PHF20L1 | 0.005063949 | −0.452694298 | 0.061 | 0.259 |
| CLTC | 0.005241062 | −0.346795887 | 0.065 | 0.259 |
| ANXA7 | 0.005253741 | −0.307240631 | 0.118 | 0.333 |
| BLVRA | 0.005473129 | −0.569307662 | 0.093 | 0.333 |
| SFPQ | 0.005522652 | 0.46513365 | 0.203 | 0.259 |
| ADRB2 | 0.005650467 | −0.329161706 | 0.061 | 0.222 |
| TPSD1 | 0.005675549 | 0.703028103 | 0.569 | 0.37 |
| FTH1 | 0.005696934 | 0.267411632 | 0.898 | 1 |
| TMEM66 | 0.005706228 | 0.267038634 | 0.321 | 0.444 |
| ASAH1 | 0.005725928 | −0.449040765 | 0.35 | 0.667 |
| COMMD6 | 0.006180168 | −0.319833912 | 0.297 | 0.593 |
| MGME1 | 0.006222017 | −0.647924783 | 0.033 | 0.222 |
| MIER1 | 0.006269812 | −0.543286139 | 0.142 | 0.407 |
| ZNF33A | 0.006366321 | −0.610332406 | 0.033 | 0.222 |
| ASL | 0.006405212 | −1.006463125 | 0.045 | 0.259 |
| FAM111A | 0.006557653 | −0.323638169 | 0.061 | 0.222 |
| PPDPF | 0.006715278 | −0.491005362 | 0.224 | 0.519 |
| UQCRQ | 0.006747342 | 0.325186054 | 0.337 | 0.407 |
| NOP56 | 0.006772512 | −0.596551447 | 0.069 | 0.259 |
| PGRMC1 | 0.006899283 | −0.511365793 | 0.057 | 0.259 |
| ZDHHC12 | 0.006956454 | −0.695218969 | 0.033 | 0.222 |
| ARF4 | 0.007099865 | −0.441328686 | 0.061 | 0.259 |
| LAMTOR4 | 0.007250151 | −0.568641807 | 0.264 | 0.593 |
| TMEM141 | 0.007441721 | −0.559281057 | 0.037 | 0.222 |
| DUSP23 | 0.007522351 | −0.335816343 | 0.089 | 0.259 |
| SPCS3 | 0.00766722 | 1.12434254 | 0.317 | 0.148 |
| GNB2L1 | 0.007761485 | −0.336331854 | 0.569 | 0.852 |
| COPE | 0.007856851 | −0.294968705 | 0.102 | 0.296 |
| VMP1 | 0.008027112 | −0.258214068 | 0.215 | 0.444 |
| PSMD4 | 0.008269146 | −0.347084492 | 0.089 | 0.259 |
| NDUFB4 | 0.008327673 | −0.332895248 | 0.106 | 0.296 |
| TSPAN4 | 0.008333815 | −0.653877215 | 0.037 | 0.222 |
| THRAP3 | 0.008480357 | −0.437134272 | 0.114 | 0.333 |
| SH3BGRL3 | 0.008572179 | 0.407412157 | 0.451 | 0.519 |
| JUP | 0.00874194 | −0.436312433 | 0.045 | 0.222 |
| PGD | 0.008762149 | −0.336228601 | 0.057 | 0.222 |
| EIF3F | 0.008783982 | −0.327175606 | 0.142 | 0.296 |
| RPL7A | 0.008900097 | −0.502184938 | 0.484 | 0.815 |
| TSPAN1 | 0.00928385 | −0.629250515 | 0.053 | 0.259 |
| USP16 | 0.009327026 | −0.371316399 | 0.102 | 0.296 |
| TM9SF3 | 0.009390267 | −0.285942249 | 0.102 | 0.296 |
| RAB14 | 0.009414507 | −0.303124676 | 0.077 | 0.222 |
| SSB | 0.009474122 | −0.545555759 | 0.093 | 0.296 |
| TRIP12 | 0.009534823 | −0.372351732 | 0.069 | 0.259 |
| PRSS23 | 0.009699391 | −0.989612654 | 0.065 | 0.296 |
| HPGDS | 0.009789142 | 0.845365732 | 0.711 | 0.481 |
| ZFP36L1 | 0.009812586 | −0.438939174 | 0.236 | 0.481 |
| HSBP1 | 0.009814822 | −0.328527278 | 0.146 | 0.37 |
| CMTM6 | 0.009861155 | −0.301071327 | 0.199 | 0.407 |
| GTF2I | 0.010053673 | −0.395681518 | 0.057 | 0.222 |
| F3 | 0.010306639 | −0.586808313 | 0.098 | 0.333 |
| FAM96B | 0.010540236 | 0.523229883 | 0.163 | 0.222 |
| PLD3 | 0.010628148 | 0.419873052 | 0.203 | 0.259 |
| UBE2L3 | 0.010686351 | −0.593254863 | 0.085 | 0.296 |
| ALCAM | 0.010841562 | −0.593759414 | 0.057 | 0.259 |
| DNAJA1 | 0.011024707 | 0.365032318 | 0.459 | 0.519 |
| CHMP5 | 0.011149431 | −0.301104368 | 0.106 | 0.296 |
| SENP6 | 0.011319908 | −0.344769945 | 0.077 | 0.222 |
| HNRNPDL | 0.011373593 | 0.292355149 | 0.232 | 0.296 |
| EIF3K | 0.011434131 | −0.356238714 | 0.256 | 0.519 |
| RPS27A | 0.011517748 | −0.341743452 | 0.846 | 1 |
| ALDH3A1 | 0.011652944 | −1.183480768 | 0.041 | 0.222 |
| GAPVD1 | 0.0119021 | −0.523996122 | 0.045 | 0.222 |
| RHOA | 0.011991924 | 0.351832166 | 0.305 | 0.37 |
| PJA2 | 0.012072559 | −0.401738343 | 0.081 | 0.259 |
| MIR205HG | 0.012245865 | −0.730246246 | 0.053 | 0.259 |
| NACA2 | 0.012467729 | −0.331551418 | 0.134 | 0.333 |
| BCAS2 | 0.0125834 | −0.735920266 | 0.061 | 0.259 |
| PTGS2 | 0.012673685 | 0.749037921 | 0.581 | 0.481 |
| TNFAIP3 | 0.012815941 | 0.755887238 | 0.663 | 0.556 |
| SDPR | 0.0130167 | −0.953223745 | 0.138 | 0.37 |
| IFI27 | 0.013046109 | −0.930068185 | 0.069 | 0.296 |
| DYNC1LI1 | 0.013304039 | −0.655767315 | 0.037 | 0.222 |
| SYPL1 | 0.01351438 | −0.544957844 | 0.065 | 0.259 |
| GBAS | 0.013525901 | −0.702330642 | 0.037 | 0.222 |
| TMED2 | 0.013746926 | −0.490223349 | 0.163 | 0.407 |
| CTSS | 0.013779035 | −0.455337888 | 0.199 | 0.407 |
| EDARADD | 0.014080335 | −0.347665779 | 0.053 | 0.222 |
| NDUFA6 | 0.014373633 | 0.398704107 | 0.207 | 0.259 |
| ABHD2 | 0.014572356 | −0.330417264 | 0.077 | 0.222 |
| RSL24D1 | 0.014747059 | −0.551949333 | 0.15 | 0.407 |
| SOD1 | 0.014750095 | −0.378766517 | 0.203 | 0.444 |
| PDHB | 0.014795019 | −0.58777695 | 0.045 | 0.222 |
| NR4A3 | 0.014932134 | 0.856701913 | 0.24 | 0.111 |
| SLC38A2 | 0.015052808 | −0.484453621 | 0.183 | 0.444 |
| KLF5 | 0.015460879 | −0.686309862 | 0.037 | 0.222 |
| ACTR2 | 0.015703842 | −0.351772093 | 0.232 | 0.444 |
| CLNK | 0.015833292 | 0.748908597 | 0.203 | 0.148 |
| KIT | 0.016202247 | −0.314462113 | 0.61 | 0.815 |
| COX6A1 | 0.016504364 | −0.440393373 | 0.268 | 0.556 |
| PRPF6 | 0.016555936 | −0.427659194 | 0.053 | 0.222 |
| MT-RNR1 | 0.016641883 | 0.461452678 | 0.813 | 0.889 |
| TPR | 0.016828793 | −0.260555691 | 0.13 | 0.296 |
| SCAF11 | 0.016869157 | −0.435035446 | 0.171 | 0.407 |
| ATP5G3 | 0.017373991 | −0.404464498 | 0.207 | 0.444 |
| PPAP2A | 0.017410123 | 1.423615268 | 0.276 | 0.037 |
| ARHGAP18 | 0.017551004 | 0.347063441 | 0.256 | 0.333 |
| PRDX6 | 0.017603358 | 0.28696063 | 0.276 | 0.37 |
| AFG3L2 | 0.019400239 | −0.263420602 | 0.081 | 0.222 |
| CSF1 | 0.019810779 | 1.407629956 | 0.272 | 0.074 |
| EIF2A | 0.019956444 | −0.435874769 | 0.077 | 0.222 |
| PRDX1 | 0.019978095 | −0.658921492 | 0.293 | 0.593 |
| CD44 | 0.020417989 | −0.321651165 | 0.394 | 0.63 |
| SMIM7 | 0.020474894 | −0.283404691 | 0.065 | 0.222 |
| ANAPC11 | 0.020668836 | −0.408696522 | 0.195 | 0.407 |
| CD74 | 0.020695899 | −0.897743993 | 0.183 | 0.407 |
| C6orf48 | 0.020702722 | 0.452446073 | 0.203 | 0.259 |
| RPL18A | 0.020921542 | −0.409748237 | 0.333 | 0.63 |
| TIAL1 | 0.020953364 | −0.320590752 | 0.065 | 0.222 |
| AKAP12 | 0.021199155 | 0.279145545 | 0.211 | 0.296 |
| CTD-3203P2.2 | 0.02128602 | −0.396243429 | 0.093 | 0.259 |
| RPS27L | 0.021324366 | −0.3289916 | 0.232 | 0.481 |
| TMBIM1 | 0.021570831 | −0.589359202 | 0.102 | 0.333 |
| RHBDD2 | 0.021636638 | −0.389253409 | 0.15 | 0.37 |
| STOML2 | 0.02192507 | −0.411952408 | 0.057 | 0.222 |
| ATG12 | 0.022091222 | −0.262556851 | 0.093 | 0.259 |
| TFF3 | 0.022119782 | 0.428932488 | 0.159 | 0.259 |
| GAS5 | 0.02261954 | −0.280144114 | 0.386 | 0.667 |
| PARP1 | 0.02291618 | 0.259568453 | 0.146 | 0.222 |
| CCDC59 | 0.022972395 | −0.294667254 | 0.098 | 0.259 |
| GLUL | 0.023407655 | 0.618651612 | 0.687 | 0.63 |
| GPR183 | 0.023410413 | −0.631201447 | 0.163 | 0.407 |
| PSMA7 | 0.023626328 | −0.262793496 | 0.252 | 0.481 |
| NDUFS5 | 0.023674987 | −0.275758019 | 0.175 | 0.37 |
| RPS7 | 0.024123211 | −0.376711904 | 0.443 | 0.741 |
| TIMM13 | 0.025105281 | −0.562966737 | 0.049 | 0.222 |
| TUBGCP2 | 0.025493263 | −0.53506103 | 0.057 | 0.222 |
| GNG5 | 0.025536472 | −0.356094462 | 0.093 | 0.259 |
| SMEK2 | 0.025990955 | −0.281622347 | 0.098 | 0.259 |
| TRIP11 | 0.0265992 | −0.409208165 | 0.065 | 0.222 |
| MOB1A | 0.026866597 | 0.265490815 | 0.215 | 0.296 |
| BACE2 | 0.027107583 | −0.515133442 | 0.073 | 0.222 |
| SNHG5 | 0.02720306 | 0.27234404 | 0.443 | 0.519 |
| BST2 | 0.027628226 | −0.254485735 | 0.228 | 0.444 |
| TCF25 | 0.027951453 | −0.275534573 | 0.098 | 0.259 |
| RAB6A | 0.028016852 | −0.591395398 | 0.065 | 0.259 |
| SLC9A3R1 | 0.028451725 | −0.267949274 | 0.073 | 0.222 |
| DUT | 0.028455805 | −0.391771232 | 0.13 | 0.333 |
| CD99 | 0.028685359 | −0.50583818 | 0.191 | 0.444 |
| BCL2A1 | 0.029059556 | 0.323564856 | 0.22 | 0.296 |
| HNRNPD | 0.029186373 | −0.449959823 | 0.098 | 0.296 |
| RPS10 | 0.02926188 | −0.312779315 | 0.228 | 0.444 |
| FLU | 0.030287294 | −0.278970371 | 0.081 | 0.222 |
| EIF3M | 0.030421364 | −0.324211861 | 0.089 | 0.259 |
| MT-CO2 | 0.03056894 | 0.603275755 | 0.581 | 0.519 |
| TMEM256 | 0.03061106 | −0.316324593 | 0.069 | 0.222 |
| RBM17 | 0.03114185 | −0.312135654 | 0.073 | 0.222 |
| CMA1 | 0.031979884 | −0.394395544 | 0.159 | 0.333 |
| KLHL9 | 0.032341509 | −0.600635274 | 0.049 | 0.222 |
| GPX1 | 0.032969039 | −0.278189937 | 0.309 | 0.444 |
| IGHA2 | 0.033208548 | 1.807345413 | 0.236 | 0.111 |
| ADH5 | 0.033237714 | −0.476773573 | 0.049 | 0.222 |
| TAGLN2 | 0.034285966 | −0.314162026 | 0.398 | 0.667 |
| MAP3K8 | 0.034820434 | 0.900615204 | 0.22 | 0.111 |
| HDLBP | 0.035829408 | −0.362867503 | 0.134 | 0.333 |
| OST4 | 0.036189414 | −0.380620698 | 0.305 | 0.556 |
| ALDH1A1 | 0.036261806 | −0.615570881 | 0.187 | 0.444 |
| RPL30 | 0.037081022 | −0.26380987 | 0.866 | 0.963 |
| PTPRC | 0.03740672 | 0.691610604 | 0.224 | 0.111 |
| PPP1R12A | 0.037823203 | −0.363380728 | 0.114 | 0.259 |
| MED4 | 0.038045135 | −0.668619264 | 0.089 | 0.296 |
| LY6E | 0.038697186 | −0.428938339 | 0.085 | 0.259 |
| DDX24 | 0.038831845 | −0.324233075 | 0.118 | 0.296 |
| ALAS1 | 0.039405646 | 1.100244794 | 0.419 | 0.185 |
| AQP3 | 0.040695953 | −1.002132699 | 0.154 | 0.259 |
| SYF2 | 0.04365277 | −0.416167705 | 0.134 | 0.333 |
| SLC25A5 | 0.044854032 | −0.409353547 | 0.203 | 0.407 |
| SOCS3 | 0.045089679 | −0.518019412 | 0.081 | 0.259 |
| CLTA | 0.046028677 | −0.419062189 | 0.106 | 0.296 |
| WASF2 | 0.046296884 | −0.482843056 | 0.057 | 0.222 |
| RAB8A | 0.049693883 | −0.27808546 | 0.077 | 0.222 |
| DYNLRB1 | 0.049871674 | −0.483307021 | 0.081 | 0.259 |
| RNF11 | 0.05031834 | −0.72174096 | 0.061 | 0.222 |
| C8orf59 | 0.050325959 | −0.341267453 | 0.089 | 0.259 |
| HLA-F | 0.050999275 | −0.55892824 | 0.106 | 0.296 |
| RPSAP58 | 0.051128012 | −0.439215728 | 0.134 | 0.333 |
| MCFD2 | 0.051280358 | −0.383693967 | 0.102 | 0.259 |
| SLC25A6 | 0.051634696 | −0.302080017 | 0.313 | 0.556 |
| ELF3 | 0.052584282 | −0.878094623 | 0.081 | 0.222 |
| LARP7 | 0.053844777 | −0.390068009 | 0.069 | 0.222 |
| TIMP1 | 0.053939892 | 0.790729866 | 0.313 | 0.185 |
| TMEM261 | 0.055373149 | −0.449881585 | 0.069 | 0.222 |
| HINT1 | 0.055880872 | −0.286044894 | 0.183 | 0.37 |
| ZEB2 | 0.057443403 | −0.45658804 | 0.171 | 0.37 |
| ID1 | 0.059387629 | −0.254784318 | 0.114 | 0.259 |
| CST3 | 0.059781029 | −0.59474929 | 0.272 | 0.444 |
| LEPROTL1 | 0.060006437 | −0.312234701 | 0.114 | 0.259 |
| PYCARD | 0.06052808 | −0.381170437 | 0.085 | 0.259 |
| SNRPE | 0.061526608 | −0.49246235 | 0.061 | 0.222 |
| TXN2 | 0.061884416 | −0.428753557 | 0.065 | 0.222 |
| TMEM123 | 0.061991196 | −0.351000588 | 0.146 | 0.333 |
| LEPROT | 0.063558142 | −0.389330727 | 0.122 | 0.296 |
| ITM2C | 0.063629605 | −0.508259775 | 0.13 | 0.333 |
| U2SURP | 0.064290419 | −0.384021362 | 0.098 | 0.259 |
| KRT19 | 0.065241631 | −0.848447637 | 0.207 | 0.407 |
| CCT4 | 0.066159022 | −0.305387169 | 0.073 | 0.222 |
| KARS | 0.066477337 | −0.41663161 | 0.065 | 0.222 |
| DNAJC19 | 0.066669699 | −0.425853219 | 0.077 | 0.222 |
| ACTN4 | 0.066785879 | −0.26911252 | 0.142 | 0.296 |
| RPL18 | 0.067211365 | −0.250658998 | 0.528 | 0.778 |
| POLR2K | 0.067264524 | −0.459423742 | 0.065 | 0.222 |
| FOXP1 | 0.068537258 | −0.363082176 | 0.248 | 0.481 |
| TTC3 | 0.070468816 | −0.379364335 | 0.093 | 0.259 |
| BATF | 0.071464488 | −0.571590037 | 0.061 | 0.222 |
| LSM4 | 0.072001504 | −0.3792638 | 0.098 | 0.259 |
| SRPR | 0.072337398 | −0.578787121 | 0.085 | 0.259 |
| DENR | 0.07235276 | −0.324195418 | 0.081 | 0.222 |
| RPL9 | 0.074220087 | −0.430080486 | 0.447 | 0.667 |
| ATP1B1 | 0.076660296 | −0.667820232 | 0.102 | 0.296 |
| EPAS1 | 0.07682875 | −0.68043095 | 0.191 | 0.407 |
| GMFG | 0.077066595 | −0.381730308 | 0.138 | 0.296 |
| RABAC1 | 0.082123898 | −0.333344291 | 0.122 | 0.296 |
| VPS4B | 0.08279529 | −0.499766913 | 0.073 | 0.222 |
| RGS2 | 0.084085373 | −0.421450146 | 0.533 | 0.704 |
| MAN1A2 | 0.084104804 | −0.393513132 | 0.073 | 0.222 |
| PSMD8 | 0.086395526 | −0.35695422 | 0.098 | 0.222 |
| EID1 | 0.08653448 | −0.336603385 | 0.285 | 0.519 |
| SQSTM1 | 0.087082319 | 0.629266563 | 0.476 | 0.37 |
| RSRC2 | 0.089852104 | −0.551193121 | 0.089 | 0.259 |
| RPL41 | 0.090777493 | 0.360943683 | 0.854 | 0.778 |
| TBCA | 0.09763818 | −0.287119046 | 0.093 | 0.222 |
| HLA-DRA | 0.10107325 | −0.578654028 | 0.11 | 0.222 |
| SH3KBP1 | 0.102515635 | −0.586254506 | 0.126 | 0.296 |
| RPL26 | 0.103774195 | −0.349711159 | 0.732 | 0.889 |
| RAB7A | 0.104857914 | −0.603608191 | 0.134 | 0.333 |
| LINC00657 | 0.104948493 | −0.303524627 | 0.228 | 0.407 |
| UBTF | 0.105131935 | −0.304457974 | 0.085 | 0.222 |
| RPS12 | 0.106636696 | −0.324294991 | 0.878 | 0.963 |
| ABI1 | 0.108115429 | −0.33790171 | 0.093 | 0.222 |
| EIF3H | 0.108282933 | −0.252407356 | 0.228 | 0.407 |
| CYC1 | 0.109337723 | −0.253590782 | 0.098 | 0.222 |
| RPL38 | 0.109569696 | −0.269045585 | 0.679 | 0.889 |
| RPL4 | 0.111060969 | −0.345086028 | 0.52 | 0.704 |
| RPLP0 | 0.111893393 | −0.344467531 | 0.74 | 0.889 |
| SLC2A3 | 0.120267808 | 0.288441199 | 0.268 | 0.148 |
| KRT18 | 0.122178114 | −0.537211188 | 0.077 | 0.222 |
| WDR1 | 0.122363912 | −0.423013966 | 0.102 | 0.222 |
| KRT8 | 0.125171983 | −0.819110947 | 0.069 | 0.222 |
| RPL10A | 0.12621152 | −0.352950866 | 0.504 | 0.741 |
| ACIN1 | 0.12910892 | −0.295289638 | 0.126 | 0.222 |
| SETD2 | 0.129315796 | −0.425841337 | 0.098 | 0.259 |
| RPL27 | 0.13206191 | −0.329612769 | 0.841 | 0.926 |
| C19orf43 | 0.136097337 | −0.569364817 | 0.093 | 0.259 |
| SLC1A5 | 0.136446683 | −0.398631214 | 0.081 | 0.222 |
| ATP5EP2 | 0.136844978 | −0.403257145 | 0.11 | 0.259 |
| RHOC | 0.136937188 | −0.31118148 | 0.089 | 0.222 |
| SERPINB3 | 0.137271175 | −1.088235395 | 0.15 | 0.259 |
| GLO1 | 0.142009038 | −0.435909004 | 0.085 | 0.222 |
| RPS18 | 0.147196377 | −0.287570809 | 0.911 | 0.963 |
| HSPB1 | 0.154695905 | −0.460991268 | 0.167 | 0.333 |
| SMYD3 | 0.159943303 | −0.348454254 | 0.118 | 0.259 |
| MTRNR2L6 | 0.159955373 | −0.4092684 | 0.163 | 0.296 |
| EIF4A1 | 0.162340898 | −0.352822934 | 0.118 | 0.259 |
| DDX17 | 0.163442821 | −0.327018636 | 0.252 | 0.444 |
| TMX4 | 0.167506275 | −0.292946321 | 0.159 | 0.259 |
| COX5A | 0.173081659 | −0.401595088 | 0.159 | 0.333 |
| SLC26A2 | 0.176689332 | −0.256101545 | 0.102 | 0.222 |
| EEF1D | 0.185470416 | −0.301564923 | 0.317 | 0.481 |
| NFKBIZ | 0.193725407 | 0.390016314 | 0.752 | 0.63 |
| HDC | 0.19468584 | 0.482711613 | 0.61 | 0.556 |
| HEXIM1 | 0.195115412 | −0.281315955 | 0.134 | 0.259 |
| COX5B | 0.19871331 | −0.271701093 | 0.24 | 0.407 |
| PTPN1 | 0.201400626 | −0.334342239 | 0.098 | 0.222 |
| TRIM24 | 0.213310106 | −0.737918673 | 0.081 | 0.222 |
| HMGB1 | 0.215052049 | −0.289603789 | 0.272 | 0.444 |
| ANXA2 | 0.217359766 | −0.60210832 | 0.13 | 0.296 |
| CTNNBL1 | 0.218335719 | −0.28950375 | 0.102 | 0.222 |
| EGR3 | 0.223617358 | 0.73810087 | 0.35 | 0.222 |
| TRAPPC3 | 0.226488035 | −0.325259994 | 0.098 | 0.222 |
| TMEM230 | 0.23414291 | −0.408108864 | 0.114 | 0.259 |
| H2AFZ | 0.237218381 | −0.552847587 | 0.134 | 0.296 |
| SPINT2 | 0.248521654 | −0.311212783 | 0.191 | 0.296 |
| ID3 | 0.259899043 | −0.596951175 | 0.085 | 0.222 |
| MT-CO1 | 0.265703339 | 0.536651999 | 0.614 | 0.519 |
| FXYD3 | 0.265937211 | −0.39737264 | 0.142 | 0.296 |
| ATP1A1 | 0.271142464 | −0.311677622 | 0.098 | 0.222 |
| CD37 | 0.304505906 | 0.565703182 | 0.232 | 0.148 |
| NDFIP2 | 0.305607199 | 0.66999462 | 0.211 | 0.111 |
| IFITM2 | 0.310963121 | 0.411342375 | 0.341 | 0.259 |
| DNTTIP2 | 0.326113817 | −0.260190104 | 0.114 | 0.222 |
| PKM | 0.331409124 | −0.253177426 | 0.329 | 0.481 |
| HNRNPA0 | 0.344828466 | −0.255856106 | 0.179 | 0.296 |
| MCL1 | 0.36289302 | −0.280466776 | 0.285 | 0.444 |
| CD83 | 0.366775011 | 0.723497531 | 0.268 | 0.148 |
| PSMD1 | 0.381588563 | −0.286602016 | 0.11 | 0.222 |
| PERP | 0.405497468 | −0.58028312 | 0.126 | 0.222 |
| BMP2K | 0.416261095 | −0.256361809 | 0.134 | 0.222 |
| GPR65 | 0.460648295 | 0.421043698 | 0.423 | 0.296 |
| CTNNB1 | 0.467258787 | −0.283146734 | 0.122 | 0.222 |
| DDX6 | 0.470011034 | −0.29515213 | 0.114 | 0.222 |
| PAFAH1B1 | 0.483368112 | −0.417301178 | 0.114 | 0.222 |
| COX4I1 | 0.48860329 | −0.25746365 | 0.439 | 0.593 |
| TMBIM6 | 0.493178566 | −0.321788873 | 0.301 | 0.444 |
| BLOC1S6 | 0.541367388 | −0.446916279 | 0.118 | 0.222 |
| PLIN2 | 0.60154964 | 0.518297761 | 0.215 | 0.148 |
| EFCAB14 | 0.622071397 | −0.352415125 | 0.215 | 0.333 |
| SLFN5 | 0.628273464 | −0.352280664 | 0.13 | 0.222 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 9.

TABLE 9

| | p_val | avg_diff | pct. 1 | pct. 2 |
|---|---|---|---|---|
| STATH | 8.98E−296 | −3.229499718 | 0.075 | 0.921 |
| ZG16B | 2.52E−103 | −1.332415704 | 0.02 | 0.44 |
| LYZ | 1.08E−98 | −1.777393223 | 0.086 | 0.601 |
| BPIFA1 | 1.18E−75 | −1.023225011 | 0.324 | 0.813 |
| IGKC | 1.62E−72 | −1.604864519 | 0.608 | 0.696 |
| LTF | 1.44E−62 | −1.085636774 | 0.021 | 0.32 |
| POSTN | 2.35E−56 | 2.113555943 | 0.519 | 0.089 |
| MSMB | 9.79E−51 | −0.708500621 | 0.044 | 0.329 |
| JUN | 1.04E−46 | −0.815603294 | 0.727 | 0.937 |
| KLF6 | 1.60E−45 | −0.951542779 | 0.505 | 0.813 |
| CCND2 | 5.75E−45 | 1.618240478 | 0.435 | 0.082 |
| CCL3 | 8.56E−41 | −1.812390124 | 0.078 | 0.288 |
| BPIFB1 | 1.86E−36 | −0.65927245 | 0.257 | 0.62 |
| NFKBIA | 1.34E−28 | −0.670716764 | 0.57 | 0.832 |
| TNFAIP3 | 1.61E−28 | −0.88221894 | 0.201 | 0.494 |
| SRGN | 2.91E−28 | −0.632623283 | 0.603 | 0.851 |
| CCL4 | 3.59E−28 | −1.368637971 | 0.099 | 0.234 |
| IGHG4 | 1.16E−27 | 1.080592367 | 0.71 | 0.399 |
| IGHG3 | 9.15E−26 | −0.550919412 | 0.692 | 0.478 |
| BIRC3 | 1.78E−25 | −0.671932636 | 0.206 | 0.5 |
| PIGR | 5.95E−24 | −0.66679958 | 0.105 | 0.342 |
| RPL18A | 1.13E−23 | 0.657456654 | 0.506 | 0.405 |
| MTRNR2L1 | 4.45E−23 | 0.744449827 | 0.796 | 0.62 |
| CD79A | 9.72E−23 | −0.621171099 | 0.37 | 0.652 |
| PPP1R15A | 6.60E−22 | −0.556141782 | 0.483 | 0.759 |
| ISCU | 1.13E−21 | −0.496472623 | 0.217 | 0.487 |
| TFF3 | 3.42E−21 | 1.125498133 | 0.344 | 0.165 |
| EAF2 | 3.61E−21 | −0.531724719 | 0.238 | 0.509 |
| ALOX15 | 1.22E−20 | 1.339267421 | 0.251 | 0.07 |
| IL2RG | 2.45E−20 | −0.462014307 | 0.091 | 0.288 |
| RGS1 | 5.04E−20 | −0.610919481 | 0.586 | 0.804 |
| NUCB2 | 5.12E−20 | −0.523841394 | 0.352 | 0.633 |
| S100A2 | 5.37E−20 | 1.250763082 | 0.319 | 0.13 |
| SLPI | 1.11E−18 | −0.326509429 | 0.577 | 0.82 |
| RPS27 | 1.13E−18 | 0.612117709 | 0.561 | 0.475 |
| PPDPF | 4.57E−18 | −0.329282812 | 0.134 | 0.323 |
| TMSB4X | 4.80E−18 | 0.632770368 | 0.665 | 0.595 |
| IER2 | 6.45E−18 | −0.552212188 | 0.34 | 0.598 |
| IGHV3-21 | 7.91E−18 | 1.628129263 | 0.237 | 0.044 |
| TNFRSF17 | 1.23E−17 | −0.531274565 | 0.266 | 0.519 |
| FOSB | 1.53E−17 | −0.430973632 | 0.503 | 0.759 |
| MYL6 | 1.73E−17 | 0.286537645 | 0.676 | 0.744 |
| RPS3A | 1.17E−16 | 0.516588181 | 0.453 | 0.402 |
| SERPINB3 | 6.61E−16 | 1.200306364 | 0.394 | 0.209 |
| GPR160 | 1.07E−15 | −0.375357306 | 0.08 | 0.241 |
| TXNIP | 8.48E−15 | −0.373156349 | 0.369 | 0.608 |
| CD9 | 1.40E−14 | 0.799903508 | 0.361 | 0.222 |
| PSAP | 1.96E−14 | −0.31705978 | 0.51 | 0.747 |
| IGKV3-20 | 3.50E−14 | 0.798945019 | 0.338 | 0.142 |
| IGHV1-24 | 6.15E−14 | 2.430343932 | 0.263 | 0.095 |
| TMEM59 | 6.74E−14 | −0.256004412 | 0.559 | 0.785 |
| PELI1 | 9.07E−14 | −0.600975587 | 0.112 | 0.285 |
| OXR1 | 2.42E−13 | −0.274320198 | 0.081 | 0.215 |
| MT-CO1 | 3.25E−13 | 0.707557268 | 0.436 | 0.367 |
| HSH2D | 4.91E−13 | −0.383115938 | 0.102 | 0.263 |
| LMO4 | 5.45E−13 | −0.361378219 | 0.139 | 0.313 |
| IGHV4-39 | 6.86E−13 | 1.547538477 | 0.215 | 0.057 |
| IGHG2 | 1.76E−12 | 0.560070163 | 0.586 | 0.364 |
| CD74 | 2.59E−12 | −0.330560967 | 0.575 | 0.782 |
| YBX1 | 6.86E−12 | 0.519457936 | 0.306 | 0.247 |
| CYBA | 7.59E−12 | −0.318812516 | 0.421 | 0.639 |
| VIM | 1.17E−11 | −0.399988452 | 0.489 | 0.696 |
| GUSBP11 | 2.06E−11 | 0.491761146 | 0.215 | 0.272 |
| CDC42SE2 | 4.54E−11 | −0.309102711 | 0.128 | 0.278 |
| DNAJB9 | 7.60E−11 | −0.274083834 | 0.356 | 0.557 |
| IGHA1 | 1.15E−10 | −0.281464043 | 0.933 | 0.861 |
| MTDH | 1.29E−10 | 0.477173883 | 0.423 | 0.351 |
| HIST1H4C | 2.80E−10 | 0.707353346 | 0.25 | 0.184 |
| PABPC4 | 3.01E−10 | −0.259313899 | 0.13 | 0.269 |
| RPL30 | 4.93E−10 | −0.264443801 | 0.843 | 0.953 |
| DUSP1 | 6.57E−10 | −0.393264402 | 0.595 | 0.741 |
| TACSTD2 | 8.76E−10 | 0.764012249 | 0.268 | 0.184 |
| EGR1 | 1.17E−09 | −0.288292663 | 0.388 | 0.582 |
| LINC00657 | 1.78E−09 | −0.268212889 | 0.168 | 0.316 |
| RNA18S5 | 2.39E−09 | −0.500389009 | 0.265 | 0.405 |
| MTRNR2L3 | 3.65E−09 | 0.59633228 | 0.394 | 0.31 |
| CTSH | 7.36E−09 | −0.271791186 | 0.185 | 0.335 |
| EZR | 7.43E−09 | −0.284425469 | 0.371 | 0.557 |
| EVI2B | 8.15E−09 | −0.388181818 | 0.166 | 0.32 |
| LMNA | 8.87E−09 | −0.285243699 | 0.205 | 0.361 |
| ENAM | 1.06E−08 | −0.316874601 | 0.654 | 0.81 |
| MT-ND4 | 2.38E−08 | 0.271411557 | 0.436 | 0.491 |
| ANXA1 | 4.05E−08 | 0.312827758 | 0.46 | 0.37 |
| MTRNR2L8 | 4.54E−08 | 0.50252258 | 0.719 | 0.614 |
| UBBP4 | 5.75E−08 | 0.481125805 | 0.324 | 0.222 |
| AL928768.3 | 7.55E−08 | −0.272349177 | 0.121 | 0.237 |
| S100A6 | 9.44E−08 | 0.452962956 | 0.588 | 0.528 |
| AC096579.7 | 2.73E−07 | 0.767559428 | 0.553 | 0.43 |
| MTRNR2L2 | 3.43E−07 | 0.504556735 | 0.589 | 0.528 |
| PKM | 8.08E−07 | 0.476339738 | 0.21 | 0.158 |
| HLA-DRA | 8.67E−07 | 0.579232876 | 0.214 | 0.123 |
| ANKRD37 | 1.43E−06 | −0.339958329 | 0.181 | 0.316 |
| BTG1 | 1.81E−06 | −0.299615301 | 0.224 | 0.364 |
| IRF4 | 3.58E−06 | −0.253961649 | 0.147 | 0.259 |
| GLCCI1 | 6.31E−06 | −0.271498852 | 0.128 | 0.241 |
| DUSP2 | 7.51E−06 | −0.393146249 | 0.168 | 0.288 |
| CITED2 | 8.41E−06 | −0.253328996 | 0.329 | 0.468 |
| EDEM3 | 8.55E−06 | −0.30310625 | 0.131 | 0.244 |
| IGHV1-18 | 3.29E−05 | 1.02654436 | 0.202 | 0.098 |
| CXCR4 | 5.16E−05 | −0.309625758 | 0.312 | 0.446 |
| IGHM | 0.000137952 | −0.258878392 | 0.295 | 0.218 |
| ATF6 | 0.000640014 | −0.252184925 | 0.119 | 0.206 |
| SRSF7 | 0.002193696 | −0.294637875 | 0.151 | 0.237 |
| IGHV3-23 | 0.010264208 | 0.594740192 | 0.242 | 0.161 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 10.

TABLE 10

| | p_val | avg_diff | pct. 1 | pct. 2 |
|---|---|---|---|---|
| STATH | 6.87E−147 | −5.172900196 | 0.017 | 0.844 |
| BPIFA1 | 1.47E−72 | −3.320482948 | 0.115 | 0.729 |
| SLPI | 9.04E−72 | −2.269622309 | 0.312 | 0.874 |
| LYZ | 2.56E−68 | −3.927326129 | 0.026 | 0.523 |
| ZG16B | 8.08E−64 | −3.273150302 | 0.009 | 0.457 |
| PIGR | 1.71E−42 | −2.165543333 | 0.055 | 0.467 |
| AQP3 | 7.53E−39 | −1.255967777 | 0.132 | 0.573 |
| MSMB | 2.83E−38 | −2.0501437 | 0.021 | 0.352 |
| LTF | 8.63E−37 | −2.650951842 | 0.011 | 0.307 |
| KRT19 | 2.95E−35 | −1.236349357 | 0.18 | 0.633 |
| RPS27 | 3.11E−35 | 1.150304146 | 0.828 | 0.683 |
| WFDC2 | 1.03E−34 | −1.487527328 | 0.117 | 0.528 |
| BPIFB1 | 3.51E−34 | −2.074791471 | 0.121 | 0.513 |
| AZGP1 | 1.78E−33 | −2.129876975 | 0.004 | 0.246 |
| DMBT1 | 1.39E−32 | −1.607971321 | 0.001 | 0.226 |
| C6orf58 | 1.95E−32 | −2.467564956 | 0 | 0.216 |
| TMSB4X | 5.59E−32 | 0.611320191 | 0.957 | 0.905 |
| RPL18A | 2.29E−31 | 0.911103919 | 0.561 | 0.462 |
| ALDH1A1 | 3.84E−31 | −1.148417831 | 0.059 | 0.412 |
| PIP | 7.71E−31 | −2.357956083 | 0 | 0.206 |
| AGR2 | 7.80E−31 | −1.333816816 | 0.1 | 0.477 |
| ELF3 | 3.18E−30 | −0.986347475 | 0.091 | 0.462 |
| IGHA1 | 3.45E−30 | 0.403737676 | 0.577 | 0.322 |
| FAM3D | 2.82E−29 | −0.833214556 | 0.007 | 0.241 |
| EGR1 | 3.91E−29 | −0.916557699 | 0.186 | 0.593 |
| SRGN | 3.97E−29 | 0.888318473 | 0.757 | 0.668 |
| RP11-1143G9.4 | 1.60E−28 | −1.838294257 | 0.002 | 0.211 |
| ADIRF | 1.93E−28 | −0.869597915 | 0.057 | 0.372 |
| FXYD3 | 8.39E−28 | −0.815732942 | 0.139 | 0.508 |
| CXCL17 | 1.49E−27 | −1.123197747 | 0.057 | 0.382 |
| RPL13A | 1.04E−26 | 0.611412045 | 0.953 | 0.899 |
| RPL10 | 3.08E−26 | 0.778735276 | 0.733 | 0.638 |
| CTSB | 3.24E−26 | −0.685011685 | 0.164 | 0.528 |
| PERP | 4.31E−26 | −0.929976605 | 0.141 | 0.508 |
| KRT7 | 1.04E−25 | −0.936887145 | 0.039 | 0.322 |
| MYL6 | 2.18E−25 | 0.298029639 | 0.639 | 0.784 |
| ALCAM | 3.56E−25 | −0.917985495 | 0.046 | 0.332 |
| SCGB3A1 | 6.42E−25 | −1.904937952 | 0.006 | 0.201 |
| SPINT2 | 1.21E−24 | −0.775885998 | 0.093 | 0.412 |
| JUN | 1.54E−24 | −0.67933947 | 0.326 | 0.739 |
| SNRPD2 | 2.11E−24 | 0.299471899 | 0.4 | 0.523 |
| KRT8 | 3.01E−24 | −1.125657751 | 0.076 | 0.392 |
| PSAP | 3.72E−24 | −0.273257558 | 0.181 | 0.462 |
| HES1 | 3.98E−24 | −1.065764602 | 0.068 | 0.367 |
| ZFP36L1 | 5.96E−24 | −0.650217767 | 0.212 | 0.573 |
| OAZ1 | 6.64E−24 | 0.462268014 | 0.399 | 0.467 |
| CLU | 8.29E−24 | −0.964743628 | 0.039 | 0.307 |
| PRSS23 | 1.09E−23 | −0.880187462 | 0.107 | 0.427 |
| SLC25A6 | 1.16E−23 | 0.26649144 | 0.35 | 0.477 |
| ID1 | 1.70E−23 | −0.966460769 | 0.115 | 0.457 |
| RPL9 | 2.05E−23 | 0.381880616 | 0.573 | 0.683 |
| CD63 | 2.75E−23 | −0.41703427 | 0.216 | 0.538 |
| RPL22 | 3.45E−23 | 0.425373313 | 0.343 | 0.422 |
| NDRG2 | 3.96E−23 | −0.796690145 | 0.018 | 0.236 |
| ATP5E | 4.83E−23 | 0.354863751 | 0.572 | 0.688 |
| CD52 | 5.31E−23 | 0.583202935 | 0.713 | 0.638 |
| RPL17 | 7.04E−23 | 0.863660795 | 0.33 | 0.276 |
| XBP1 | 7.08E−23 | −1.022085869 | 0.117 | 0.457 |
| TACSTD2 | 1.11E−22 | −0.582431196 | 0.151 | 0.462 |
| DDX17 | 2.74E−22 | −0.307977225 | 0.217 | 0.508 |
| EHF | 3.03E−22 | −0.782792124 | 0.042 | 0.291 |
| TSC22D1 | 5.82E−22 | −0.756357354 | 0.103 | 0.407 |
| MT-CO3 | 5.85E−22 | −0.425955875 | 0.401 | 0.698 |
| ATP1B1 | 7.30E−22 | −0.911959448 | 0.069 | 0.357 |
| PPDPF | 9.37E−22 | −0.380486135 | 0.148 | 0.417 |
| LGALS3 | 1.05E−21 | −0.688493403 | 0.23 | 0.573 |
| CD9 | 1.22E−21 | −0.50962867 | 0.177 | 0.482 |
| YWHAE | 1.26E−21 | −0.353287774 | 0.114 | 0.352 |
| AQP5 | 3.06E−21 | −0.898530421 | 0.026 | 0.256 |
| EPAS1 | 3.39E−21 | −1.134825437 | 0.071 | 0.357 |
| GSTP1 | 3.88E−21 | −0.389900019 | 0.249 | 0.558 |
| RPL24 | 5.11E−21 | 0.294390051 | 0.719 | 0.834 |
| ATP1A1 | 5.45E−21 | −0.397824858 | 0.125 | 0.377 |
| NUCB2 | 6.50E−21 | −0.635568495 | 0.075 | 0.327 |
| BTG2 | 9.38E−21 | −0.438718443 | 0.249 | 0.538 |
| CTSD | 1.83E−20 | −0.339009194 | 0.136 | 0.382 |
| CD2 | 1.94E−20 | 0.262754484 | 0.433 | 0.533 |
| LCP1 | 1.97E−20 | 0.265344162 | 0.345 | 0.402 |
| TMED2 | 2.35E−20 | −0.389121389 | 0.104 | 0.327 |
| SDC4 | 4.97E−20 | −0.59117224 | 0.042 | 0.266 |
| NEAT1 | 5.27E−20 | −0.814698325 | 0.106 | 0.362 |
| IL32 | 5.64E−20 | 1.06220675 | 0.479 | 0.312 |
| TXN | 5.79E−20 | −0.426160466 | 0.225 | 0.523 |
| KRT18 | 6.64E−20 | −0.698170065 | 0.088 | 0.357 |
| GLUL | 8.25E−20 | −0.750451807 | 0.12 | 0.417 |
| PRDX1 | 9.93E−20 | −0.642196843 | 0.173 | 0.482 |
| NPM1 | 1.04E−19 | 0.353230855 | 0.335 | 0.407 |
| CD46 | 1.15E−19 | −0.479328699 | 0.109 | 0.362 |
| FOS | 1.26E−19 | −0.772239985 | 0.51 | 0.799 |
| NR4A1 | 1.27E−19 | −0.601452002 | 0.149 | 0.427 |
| CLINT1 | 1.51E−19 | −0.293391639 | 0.097 | 0.312 |
| ELL2 | 2.07E−19 | −0.376287675 | 0.05 | 0.241 |
| LINC00657 | 2.33E−19 | −0.325986644 | 0.106 | 0.327 |
| CD69 | 2.69E−19 | 0.508287734 | 0.406 | 0.487 |
| OST4 | 3.02E−19 | 0.34958719 | 0.321 | 0.417 |
| SAT1 | 3.63E−19 | −0.636818694 | 0.299 | 0.648 |
| OCIAD1 | 4.62E−19 | −0.452827984 | 0.106 | 0.357 |
| ALDH3A1 | 4.82E−19 | −0.967190219 | 0.052 | 0.302 |
| RPS10 | 5.52E−19 | 0.355187699 | 0.279 | 0.357 |
| KLF5 | 7.12E−19 | −0.82676946 | 0.055 | 0.302 |
| SELK | 1.02E−18 | 0.348946544 | 0.274 | 0.357 |
| COMMD6 | 1.26E−18 | 0.277851848 | 0.427 | 0.538 |
| NET1 | 2.07E−18 | −0.569741714 | 0.042 | 0.251 |
| IER2 | 2.61E−18 | −0.376479357 | 0.241 | 0.528 |
| YWHAZ | 3.02E−18 | 0.291504595 | 0.383 | 0.472 |
| RPS28 | 3.42E−18 | 0.371365382 | 0.585 | 0.643 |
| KTN1 | 3.83E−18 | −0.336776954 | 0.141 | 0.382 |
| XIST | 4.22E−18 | −0.46105177 | 0.133 | 0.387 |
| CST3 | 4.56E−18 | −0.821130668 | 0.09 | 0.352 |
| SCGB1A1 | 5.18E−18 | −1.799309814 | 0.023 | 0.211 |
| TMED10 | 5.24E−18 | −0.285660899 | 0.135 | 0.367 |
| TTC3 | 5.73E−18 | −0.255275911 | 0.074 | 0.236 |
| BTG1 | 6.12E−18 | 0.273373278 | 0.505 | 0.598 |
| RBM39 | 6.97E−18 | 0.256254726 | 0.26 | 0.392 |
| ARPC3 | 6.99E−18 | 0.3042235 | 0.249 | 0.332 |
| RPL28 | 8.58E−18 | 0.286589411 | 0.632 | 0.719 |
| IGKC | 1.00E−17 | −1.19705687 | 0.091 | 0.206 |
| GAPDH | 1.05E−17 | 0.319074363 | 0.535 | 0.618 |
| PTPRF | 1.14E−17 | −0.707825698 | 0.031 | 0.236 |
| TMX4 | 1.25E−17 | 0.388022679 | 0.144 | 0.221 |
| TM9SF3 | 1.27E−17 | −0.316034615 | 0.091 | 0.291 |
| HSPB1 | 1.41E−17 | −0.816893739 | 0.136 | 0.412 |
| RNA28S5 | 1.78E−17 | −0.346704858 | 0.55 | 0.839 |
| SSR2 | 2.15E−17 | 0.250897541 | 0.209 | 0.317 |
| SNHG6 | 2.81E−17 | 0.330493955 | 0.326 | 0.412 |
| LAMTOR4 | 2.83E−17 | 0.406562447 | 0.204 | 0.271 |
| APLP2 | 3.47E−17 | −0.604833777 | 0.115 | 0.382 |
| ANXA2 | 3.81E−17 | −0.425824682 | 0.236 | 0.513 |
| MORF4L1 | 4.33E−17 | 0.351993358 | 0.261 | 0.342 |
| LCN2 | 4.59E−17 | −0.993431258 | 0.023 | 0.206 |
| CD3D | 4.70E−17 | 0.295729645 | 0.4 | 0.472 |
| LMO4 | 5.11E−17 | −0.262366164 | 0.091 | 0.281 |
| TSPAN3 | 5.17E−17 | −0.305100265 | 0.058 | 0.226 |
| SUB1 | 7.22E−17 | 0.327742896 | 0.272 | 0.352 |
| TMED3 | 7.23E−17 | −0.463177529 | 0.053 | 0.251 |
| MBNL1 | 8.00E−17 | 0.318870042 | 0.34 | 0.392 |
| NDUFB11 | 1.42E−16 | 0.276895812 | 0.17 | 0.261 |
| SPCS3 | 1.51E−16 | 0.310971414 | 0.222 | 0.312 |
| HLA-A | 1.69E−16 | 0.30784272 | 0.69 | 0.774 |
| RHOB | 1.92E−16 | −0.60735453 | 0.047 | 0.251 |
| F3 | 2.04E−16 | −0.740124166 | 0.115 | 0.387 |
| SLC12A2 | 2.18E−16 | −0.712342669 | 0.031 | 0.221 |
| WDR83OS | 2.37E−16 | −0.252994298 | 0.095 | 0.276 |
| P4HB | 2.54E−16 | −0.348843068 | 0.112 | 0.322 |
| ATF3 | 2.83E−16 | −0.808464173 | 0.129 | 0.407 |
| S100A10 | 3.26E−16 | 0.271248449 | 0.238 | 0.327 |
| MT-ND1 | 3.34E−16 | −0.289472703 | 0.224 | 0.437 |
| CLDN4 | 3.45E−16 | −0.62967819 | 0.125 | 0.377 |
| RPL4 | 3.57E−16 | 0.293817232 | 0.692 | 0.769 |
| RTN4 | 3.61E−16 | −0.487835694 | 0.055 | 0.251 |
| S100A11 | 4.13E−16 | −0.392905205 | 0.224 | 0.472 |
| METTL7A | 4.50E−16 | −0.665509968 | 0.044 | 0.246 |
| DUSP1 | 4.58E−16 | −0.478038847 | 0.477 | 0.754 |
| IER3 | 5.08E−16 | −0.775275733 | 0.11 | 0.377 |
| RPL38 | 6.15E−16 | 0.281565578 | 0.776 | 0.874 |
| FOSB | 6.70E−16 | −0.453420201 | 0.304 | 0.603 |
| LEPROTL1 | 7.51E−16 | 0.278696067 | 0.209 | 0.271 |
| MT1X | 9.48E−16 | −0.719605904 | 0.091 | 0.327 |
| CTNNA1 | 1.01E−15 | −0.53772872 | 0.053 | 0.251 |
| RTN3 | 1.10E−15 | −0.253521866 | 0.07 | 0.241 |
| MTRNR2L12 | 1.39E−15 | −0.272517932 | 0.614 | 0.809 |
| S100A6 | 2.08E−15 | −0.485283746 | 0.604 | 0.819 |
| GOLGA4 | 3.52E−15 | −0.386800817 | 0.121 | 0.332 |
| SYPL1 | 3.63E−15 | −0.578111605 | 0.047 | 0.241 |
| PTTG1IP | 4.43E−15 | −0.310497515 | 0.088 | 0.276 |
| NPC2 | 5.02E−15 | −0.353603256 | 0.084 | 0.276 |
| NDUFC1 | 5.07E−15 | −0.28548883 | 0.075 | 0.246 |
| ASPH | 5.43E−15 | −0.404180627 | 0.04 | 0.206 |
| TCEAL4 | 5.70E−15 | −0.380477662 | 0.043 | 0.201 |
| PPCS | 5.75E−15 | −0.361085516 | 0.065 | 0.241 |
| IGHG4 | 6.18E−15 | 2.159928775 | 0.212 | 0.025 |
| PFDN5 | 7.33E−15 | 0.292347128 | 0.699 | 0.779 |
| DSTN | 7.98E−15 | −0.251432094 | 0.151 | 0.357 |
| TSPAN13 | 1.18E−14 | −0.410985135 | 0.043 | 0.211 |
| ITM2B | 1.74E−14 | −0.264272545 | 0.343 | 0.603 |
| RAN | 1.77E−14 | 0.313896595 | 0.196 | 0.271 |
| NFIB | 1.81E−14 | −0.660380084 | 0.033 | 0.216 |
| CCDC47 | 2.02E−14 | −0.307842955 | 0.058 | 0.216 |
| VDAC1 | 2.16E−14 | −0.252425702 | 0.065 | 0.221 |
| DPM3 | 2.18E−14 | −0.294563811 | 0.059 | 0.221 |
| OAT | 2.26E−14 | −0.356319838 | 0.071 | 0.246 |
| SSR4 | 3.17E−14 | 0.26587299 | 0.408 | 0.492 |
| TMEM59 | 3.51E−14 | −0.369810527 | 0.199 | 0.442 |
| CFH | 3.59E−14 | −0.522349496 | 0.039 | 0.211 |
| LAMP1 | 4.78E−14 | −0.317863679 | 0.058 | 0.211 |
| SLC31A1 | 5.03E−14 | −0.526442939 | 0.034 | 0.206 |
| PRDX6 | 5.50E−14 | −0.257047825 | 0.103 | 0.281 |
| TM4SF1 | 5.75E−14 | −0.517520608 | 0.038 | 0.201 |
| TNFSF10 | 5.81E−14 | −0.476518422 | 0.085 | 0.286 |
| EPHX1 | 6.68E−14 | −0.535151994 | 0.033 | 0.206 |
| UBBP4 | 8.86E−14 | 0.686136622 | 0.227 | 0.176 |
| ARFGAP3 | 9.30E−14 | −0.292374893 | 0.059 | 0.211 |
| TCN1 | 9.63E−14 | −1.018373419 | 0.049 | 0.246 |
| CD59 | 1.14E−13 | −0.485797044 | 0.093 | 0.302 |
| AKAP9 | 1.53E−13 | −0.324842418 | 0.14 | 0.337 |
| ADAM28 | 1.81E−13 | −0.643640855 | 0.06 | 0.261 |
| RRBP1 | 1.84E−13 | −0.319962951 | 0.074 | 0.241 |
| SEC63 | 2.11E−13 | −0.286237647 | 0.062 | 0.211 |
| MDK | 2.29E−13 | −0.535019303 | 0.046 | 0.221 |
| APP | 2.55E−13 | −0.355056289 | 0.055 | 0.221 |
| DSP | 3.14E−13 | −0.532904304 | 0.053 | 0.236 |
| CHMP3 | 3.82E−13 | −0.285526416 | 0.058 | 0.206 |
| MDH1 | 3.89E−13 | −0.311195368 | 0.097 | 0.271 |
| CLDN7 | 6.19E−13 | −0.697339874 | 0.034 | 0.206 |
| CLEC2B | 6.35E−13 | 0.347524552 | 0.257 | 0.312 |
| TMSB10 | 6.80E−13 | 0.291302483 | 0.858 | 0.915 |
| HDLBP | 9.37E−13 | −0.377676954 | 0.078 | 0.256 |
| PRDX5 | 9.44E−13 | −0.252853324 | 0.145 | 0.337 |
| PCBP2 | 1.00E−12 | −0.251405276 | 0.166 | 0.357 |
| NFKBIA | 1.15E−12 | 0.254072584 | 0.427 | 0.503 |
| FNBP1 | 1.43E−12 | 0.285759697 | 0.181 | 0.251 |
| NUPR1 | 1.49E−12 | −0.550057829 | 0.04 | 0.211 |
| ANP32B | 2.69E−12 | 0.262448751 | 0.168 | 0.231 |
| MPZL2 | 3.17E−12 | −0.502288273 | 0.047 | 0.216 |
| HNRNPA0 | 3.75E−12 | 0.289807408 | 0.141 | 0.211 |
| SUCLG1 | 6.54E−12 | −0.288420584 | 0.07 | 0.221 |
| PSMB7 | 7.00E−12 | −0.363609814 | 0.065 | 0.226 |
| DEK | 7.48E−12 | 0.309388716 | 0.167 | 0.231 |
| VIM | 1.23E−11 | 0.312663594 | 0.321 | 0.377 |
| ZRANB2 | 1.24E−11 | −0.319215508 | 0.078 | 0.241 |
| HIST1H4C | 1.31E−11 | 0.499391706 | 0.265 | 0.337 |
| MALAT1 | 1.31E−11 | −0.608717849 | 0.919 | 0.985 |
| MT-ND3 | 1.59E−11 | −0.324707246 | 0.343 | 0.563 |
| IDI1 | 1.72E−11 | 0.303439188 | 0.135 | 0.201 |
| DST | 1.77E−11 | −0.508841228 | 0.05 | 0.206 |
| TXNL1 | 1.90E−11 | −0.308345138 | 0.056 | 0.201 |
| CAP1 | 2.08E−11 | 0.254918292 | 0.265 | 0.357 |
| LDHB | 2.42E−11 | 0.270439557 | 0.228 | 0.281 |
| ZNF326 | 2.51E−11 | −0.426669369 | 0.058 | 0.216 |
| CSTB | 2.62E−11 | −0.402383822 | 0.128 | 0.317 |
| UGT2A2 | 3.06E−11 | −0.841663756 | 0.059 | 0.241 |
| DDX3X | 3.10E−11 | 0.258153708 | 0.25 | 0.327 |
| ALDH3A2 | 3.41E−11 | −0.559773025 | 0.059 | 0.231 |
| MGST1 | 4.05E−11 | −0.562052763 | 0.044 | 0.206 |
| RPL15 | 4.68E−11 | 0.274838004 | 0.875 | 0.925 |
| LIMA1 | 6.46E−11 | −0.540592419 | 0.065 | 0.236 |
| USMG5 | 1.11E−10 | 0.270404384 | 0.177 | 0.236 |
| GOLGB1 | 1.44E−10 | −0.354078792 | 0.13 | 0.317 |
| SRRM2 | 1.48E−10 | −0.274174291 | 0.107 | 0.261 |
| KLF4 | 1.56E−10 | −0.385638708 | 0.07 | 0.226 |
| NFKBIZ | 1.84E−10 | 0.385008289 | 0.179 | 0.251 |
| H2AFJ | 2.07E−10 | −0.25860277 | 0.072 | 0.211 |
| CP | 2.37E−10 | −0.704370238 | 0.07 | 0.251 |
| ERRFI1 | 2.44E−10 | −0.33032744 | 0.068 | 0.201 |
| SLC25A25 | 3.23E−10 | −0.364259655 | 0.074 | 0.221 |
| TMEM219 | 4.60E−10 | −0.357272003 | 0.082 | 0.236 |
| NCOA4 | 4.67E−10 | −0.329924333 | 0.078 | 0.231 |
| MTRNR2L6 | 4.72E−10 | −0.468878065 | 0.122 | 0.286 |
| GPR183 | 4.97E−10 | 0.832891111 | 0.251 | 0.141 |
| MTDH | 5.21E−10 | 0.297774412 | 0.16 | 0.221 |
| KRT5 | 8.29E−10 | −0.747730732 | 0.071 | 0.241 |
| ASAH1 | 8.40E−10 | −0.393981594 | 0.072 | 0.231 |
| TSPAN1 | 1.13E−09 | −0.540696157 | 0.069 | 0.231 |
| MTRNR2L11 | 1.29E−09 | −0.555605207 | 0.108 | 0.281 |
| MT1E | 1.59E−09 | −0.430742135 | 0.069 | 0.216 |
| RAC1 | 1.59E−09 | −0.259092517 | 0.081 | 0.216 |
| ARHGAP5 | 1.89E−09 | −0.276267376 | 0.075 | 0.211 |
| BAZ2B | 1.96E−09 | −0.382025486 | 0.062 | 0.201 |
| ANXA1 | 3.42E−09 | −0.292539942 | 0.493 | 0.704 |
| SGK1 | 3.64E−09 | −0.300716663 | 0.129 | 0.291 |
| SERPINB3 | 7.98E−09 | −0.734121991 | 0.16 | 0.372 |
| ETS2 | 1.03E−08 | −0.354319198 | 0.113 | 0.256 |
| VMO1 | 1.05E−08 | −0.538201138 | 0.089 | 0.251 |
| IFI27 | 1.85E−08 | −0.679413099 | 0.062 | 0.216 |
| NCOA7 | 1.89E−08 | −0.335363062 | 0.142 | 0.291 |
| GMFG | 3.85E−08 | 0.537614189 | 0.253 | 0.201 |
| MT2A | 4.05E−08 | −0.270048112 | 0.144 | 0.261 |
| ARL4A | 4.80E−08 | −0.269006102 | 0.075 | 0.201 |
| PNISR | 1.06E−07 | −0.387278327 | 0.081 | 0.206 |
| MTRNR2L5 | 1.25E−07 | −0.543584264 | 0.102 | 0.256 |
| COTL1 | 1.73E−07 | 0.58297803 | 0.21 | 0.146 |
| POSTN | 2.46E−07 | 1.249819278 | 0.227 | 0.101 |
| PRPF4B | 3.44E−07 | −0.265194008 | 0.082 | 0.201 |
| ZNF638 | 1.32E−06 | −0.29897234 | 0.084 | 0.206 |
| CREM | 3.85E−06 | 0.641100667 | 0.3 | 0.211 |
| RNA18S5 | 6.16E−06 | −0.430892384 | 0.081 | 0.206 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 11. The clusters are as shown in FIG. 1.

TABLE 11

| | myAUC | avg_diff | power | pct. 1 | pct. 2 | cluster | gene |
|---|---|---|---|---|---|---|---|
| S100A2 | 0.861 | 1.695874395 | 0.722 | 0.877 | 0.276 | Basal | S100A2 |
| KRT5 | 0.835 | 1.698301604 | 0.67 | 0.775 | 0.158 | Basal | KRT5 |
| KRT15 | 0.808 | 1.939186869 | 0.616 | 0.698 | 0.131 | Basal | KRT15 |
| POSTN | 0.767 | 1.182631755 | 0.534 | 0.798 | 0.379 | Basal | POSTN |
| MMP10 | 0.745 | 1.875165347 | 0.49 | 0.584 | 0.13 | Basal | MMP10 |
| PERP | 0.745 | 0.83937019 | 0.49 | 0.813 | 0.434 | Basal | PERP |
| AQP3 | 0.743 | 0.877291555 | 0.486 | 0.789 | 0.396 | Basal | AQP3 |
| EGR1 | 0.741 | 0.787125943 | 0.482 | 0.867 | 0.583 | Basal | EGR1 |
| CD9 | 0.737 | 0.769467798 | 0.474 | 0.844 | 0.542 | Basal | CD9 |
| MIR205HG | 0.733 | 1.342628998 | 0.466 | 0.568 | 0.125 | Basal | MIR205HG |
| F3 | 0.733 | 0.951236553 | 0.466 | 0.746 | 0.361 | Basal | F3 |
| FOS | 0.733 | 0.573230114 | 0.466 | 0.973 | 0.871 | Basal | FOS |
| TACSTD2 | 0.73 | 0.836934131 | 0.46 | 0.811 | 0.452 | Basal | TACSTD2 |
| KRT17 | 0.726 | 1.445040766 | 0.452 | 0.526 | 0.085 | Basal | KRT17 |
| ALOX15 | 0.725 | 1.030165096 | 0.45 | 0.718 | 0.33 | Basal | ALOX15 |
| ETS2 | 0.716 | 1.016120389 | 0.432 | 0.666 | 0.3 | Basal | ETS2 |
| JUNB | 0.712 | 0.631929002 | 0.424 | 0.888 | 0.714 | Basal | JUNB |
| KRT19 | 0.711 | 0.566957795 | 0.422 | 0.891 | 0.534 | Basal | KRT19 |
| DST | 0.699 | 1.203828423 | 0.398 | 0.529 | 0.156 | Basal | DST |
| TNC | 0.696 | 1.163954103 | 0.392 | 0.506 | 0.126 | Basal | TNC |
| TSC22D1 | 0.696 | 1.005871912 | 0.392 | 0.639 | 0.327 | Basal | TSC22D1 |
| ID1 | 0.696 | 0.796792097 | 0.392 | 0.718 | 0.413 | Basal | ID1 |
| TP63 | 0.683 | 1.270526795 | 0.366 | 0.412 | 0.05 | Basal | TP63 |
| LAMB3 | 0.672 | 1.231216202 | 0.344 | 0.402 | 0.067 | Basal | LAMB3 |
| CLDN1 | 0.671 | 0.997700546 | 0.342 | 0.47 | 0.14 | Basal | CLDN1 |
| IL33 | 0.671 | 0.955586883 | 0.342 | 0.502 | 0.176 | Basal | IL33 |
| ALDH3A1 | 0.671 | 0.872930557 | 0.342 | 0.529 | 0.201 | Basal | ALDH3A1 |
| SERPINF1 | 0.668 | 0.852437637 | 0.336 | 0.502 | 0.176 | Basal | SERPINF1 |
| NCOA7 | 0.668 | 0.821461148 | 0.336 | 0.633 | 0.358 | Basal | NCOA7 |
| BTF3 | 0.665 | 0.584645302 | 0.33 | 0.744 | 0.552 | Basal | BTF3 |
| FXYD3 | 0.665 | 0.5444184 | 0.33 | 0.755 | 0.476 | Basal | FXYD3 |
| PRSS23 | 0.662 | 0.534848299 | 0.324 | 0.716 | 0.416 | Basal | PRSS23 |
| ALDH3A2 | 0.659 | 0.93236929 | 0.318 | 0.478 | 0.184 | Basal | ALDH3A2 |
| SFN | 0.656 | 0.992698152 | 0.312 | 0.419 | 0.117 | Basal | SFN |
| CYR61 | 0.656 | 0.917109653 | 0.312 | 0.466 | 0.17 | Basal | CYR61 |
| ATF3 | 0.656 | 0.613569125 | 0.312 | 0.694 | 0.451 | Basal | ATF3 |
| SGK1 | 0.651 | 0.787792104 | 0.302 | 0.575 | 0.317 | Basal | SGK1 |
| RPL10A | 0.651 | 0.436814756 | 0.302 | 0.818 | 0.69 | Basal | RPL10A |
| SERPINB3 | 0.875 | 2.058155337 | 0.75 | 0.895 | 0.342 | Apical | SERPINB3 |
| KRT191 | 0.859 | 1.263387356 | 0.718 | 0.955 | 0.484 | Apical | KRT19 |
| S100A6 | 0.81 | 0.895514385 | 0.62 | 0.974 | 0.773 | Apical | S100A6 |
| AGR2 | 0.801 | 1.323965583 | 0.602 | 0.814 | 0.321 | Apical | AGR2 |
| ANXA1 | 0.799 | 1.029780227 | 0.598 | 0.934 | 0.618 | Apical | ANXA1 |
| CLDN4 | 0.792 | 1.40190062 | 0.584 | 0.784 | 0.317 | Apical | CLDN4 |
| ELF3 | 0.778 | 1.18234352 | 0.556 | 0.763 | 0.288 | Apical | ELF3 |
| SLPI | 0.778 | 0.364260498 | 0.556 | 0.976 | 0.674 | Apical | SLPI |
| WFDC2 | 0.775 | 1.334956301 | 0.55 | 0.784 | 0.352 | Apical | WFDC2 |
| PRSS231 | 0.769 | 1.011619409 | 0.538 | 0.805 | 0.363 | Apical | PRSS23 |
| KRT8 | 0.765 | 1.209145233 | 0.53 | 0.727 | 0.271 | Apical | KRT8 |
| TACSTD21 | 0.762 | 0.907492414 | 0.524 | 0.836 | 0.414 | Apical | TACSTD2 |
| HSPB1 | 0.754 | 1.077867374 | 0.508 | 0.767 | 0.379 | Apical | HSPB1 |
| VMO1 | 0.751 | 1.685967051 | 0.502 | 0.63 | 0.183 | Apical | VMO1 |
| SAT1 | 0.747 | 0.846972118 | 0.494 | 0.875 | 0.575 | Apical | SAT1 |
| KRT18 | 0.744 | 1.071999046 | 0.488 | 0.703 | 0.278 | Apical | KRT18 |
| TSPAN1 | 0.743 | 1.251216647 | 0.486 | 0.613 | 0.148 | Apical | TSPAN1 |
| GSTP1 | 0.742 | 0.775405432 | 0.484 | 0.856 | 0.559 | Apical | GSTP1 |
| SERPINB4 | 0.732 | 1.395432495 | 0.464 | 0.589 | 0.139 | Apical | SERPINB4 |
| AQP31 | 0.732 | 0.956791807 | 0.464 | 0.769 | 0.37 | Apical | AQP3 |
| UGT2A2 | 0.726 | 1.228941086 | 0.452 | 0.58 | 0.144 | Apical | UGT2A2 |
| EPAS1 | 0.725 | 1.075336005 | 0.45 | 0.658 | 0.248 | Apical | EPAS1 |
| ALDH1A1 | 0.724 | 1.004129621 | 0.448 | 0.664 | 0.269 | Apical | ALDH1A1 |
| LGALS3 | 0.723 | 0.827865867 | 0.446 | 0.825 | 0.548 | Apical | LGALS3 |
| ANXA2 | 0.723 | 0.732718179 | 0.446 | 0.831 | 0.518 | Apical | ANXA2 |
| PERP1 | 0.72 | 0.752207448 | 0.44 | 0.794 | 0.408 | Apical | PERP |
| EZR | 0.712 | 0.723021059 | 0.424 | 0.785 | 0.444 | Apical | EZR |
| CD91 | 0.708 | 0.657390499 | 0.416 | 0.837 | 0.519 | Apical | CD9 |
| TXN | 0.706 | 0.842798873 | 0.412 | 0.728 | 0.433 | Apical | TXN |
| ATP1B1 | 0.705 | 0.949966675 | 0.41 | 0.62 | 0.252 | Apical | ATP1B1 |
| F31 | 0.704 | 0.745677642 | 0.408 | 0.718 | 0.338 | Apical | F3 |
| MGST1 | 0.703 | 1.062149898 | 0.406 | 0.53 | 0.144 | Apical | MGST1 |
| ALCAM | 0.702 | 1.029675653 | 0.404 | 0.568 | 0.192 | Apical | ALCAM |
| CXCL17 | 0.702 | 0.930957801 | 0.404 | 0.613 | 0.231 | Apical | CXCL17 |
| MT1X | 0.7 | 1.005539873 | 0.4 | 0.61 | 0.232 | Apical | MT1X |
| FXYD31 | 0.699 | 0.664020973 | 0.398 | 0.784 | 0.443 | Apical | FXYD3 |
| PRDX1 | 0.695 | 0.731994052 | 0.39 | 0.745 | 0.453 | Apical | PRDX1 |
| S100P | 0.687 | 1.566773257 | 0.374 | 0.468 | 0.118 | Apical | S100P |
| GABRP | 0.687 | 1.125372632 | 0.374 | 0.455 | 0.09 | Apical | GABRP |
| NTS | 0.685 | 1.543206775 | 0.37 | 0.472 | 0.123 | Apical | NTS |
| CSTB | 0.685 | 0.909530174 | 0.37 | 0.581 | 0.251 | Apical | CSTB |
| ALOX151 | 0.681 | 0.651809834 | 0.362 | 0.675 | 0.312 | Apical | ALOX15 |
| ACTG1 | 0.679 | 0.497977283 | 0.358 | 0.864 | 0.634 | Apical | ACTG1 |
| KRT7 | 0.678 | 0.998125408 | 0.356 | 0.497 | 0.161 | Apical | KRT7 |
| CP | 0.678 | 0.943897456 | 0.356 | 0.542 | 0.207 | Apical | CP |
| HES1 | 0.678 | 0.889648712 | 0.356 | 0.608 | 0.293 | Apical | HES1 |
| S100A11 | 0.678 | 0.593789448 | 0.356 | 0.761 | 0.492 | Apical | S100A11 |
| DUSP1 | 0.675 | 0.572911453 | 0.35 | 0.859 | 0.687 | Apical | DUSP1 |
| CTSB | 0.674 | 0.547887495 | 0.348 | 0.704 | 0.416 | Apical | CTSB |
| CLDN7 | 0.67 | 0.997719251 | 0.34 | 0.434 | 0.106 | Apical | CLDN7 |
| ATF31 | 0.666 | 0.614450226 | 0.332 | 0.705 | 0.427 | Apical | ATF3 |
| ADAM28 | 0.662 | 0.89841213 | 0.324 | 0.468 | 0.158 | Apical | ADAM28 |
| KLF5 | 0.662 | 0.771503616 | 0.324 | 0.519 | 0.208 | Apical | KLF5 |
| TNFSF10 | 0.661 | 0.82900933 | 0.322 | 0.506 | 0.205 | Apical | TNFSF10 |
| SPINT2 | 0.66 | 0.633011652 | 0.32 | 0.613 | 0.329 | Apical | SPINT2 |
| CST1 | 0.656 | 2.353319071 | 0.312 | 0.39 | 0.1 | Apical | CST1 |
| CD55 | 0.656 | 0.753430949 | 0.312 | 0.607 | 0.344 | Apical | CD55 |
| LYZ | 0.969 | 4.240490414 | 0.938 | 0.969 | 0.264 | Glandular | LYZ |
| SLPI1 | 0.953 | 2.213073568 | 0.906 | 1 | 0.712 | Glandular | SLPI |
| AZGP1 | 0.952 | 3.306762257 | 0.904 | 0.911 | 0.045 | Glandular | AZGP1 |
| PIGR1 | 0.952 | 2.283257407 | 0.904 | 0.974 | 0.265 | Glandular | PIGR |
| BPIFB1 | 0.948 | 2.764430304 | 0.896 | 0.968 | 0.337 | Glandular | BPIFB1 |
| LTF | 0.946 | 3.981550238 | 0.892 | 0.905 | 0.086 | Glandular | LTF |
| ZG16B | 0.944 | 4.307312581 | 0.888 | 0.913 | 0.145 | Glandular | ZG16B |
| STATH | 0.942 | 4.910954991 | 0.884 | 0.946 | 0.323 | Glandular | STATH |
| TCN1 | 0.932 | 2.947842045 | 0.864 | 0.881 | 0.058 | Glandular | TCN1 |
| BPIFA1 | 0.925 | 3.617312293 | 0.85 | 0.956 | 0.414 | Glandular | BPIFA1 |
| PIP | 0.877 | 3.509318313 | 0.754 | 0.769 | 0.038 | Glandular | PIP |
| C6orf58 | 0.876 | 3.837502534 | 0.752 | 0.764 | 0.038 | Glandular | C6orf58 |
| DMBT1 | 0.874 | 2.863131882 | 0.748 | 0.76 | 0.029 | Glandular | DMBT1 |
| RP11-1143G9.4 | 0.857 | 3.521357437 | 0.714 | 0.745 | 0.056 | Glandular | RP11-1143G9.4 |
| ODAM | 0.824 | 2.470507976 | 0.648 | 0.663 | 0.03 | Glandular | ODAM |
| XBP1 | 0.773 | 0.945081189 | 0.546 | 0.841 | 0.41 | Glandular | XBP1 |
| CXCL171 | 0.772 | 1.066554081 | 0.544 | 0.764 | 0.263 | Glandular | CXCL17 |
| RNASE1 | 0.762 | 1.52275044 | 0.524 | 0.58 | 0.061 | Glandular | RNASE1 |
| WFDC21 | 0.745 | 0.854666933 | 0.49 | 0.839 | 0.404 | Glandular | WFDC2 |
| CCL28 | 0.739 | 1.410017835 | 0.478 | 0.497 | 0.021 | Glandular | CCL28 |
| NUCB2 | 0.735 | 0.980777634 | 0.47 | 0.662 | 0.224 | Glandular | NUCB2 |
| NDRG2 | 0.722 | 1.067994392 | 0.444 | 0.552 | 0.118 | Glandular | NDRG2 |
| SLC12A2 | 0.714 | 1.075897389 | 0.428 | 0.56 | 0.143 | Glandular | SLC12A2 |
| SCGB3A1 | 0.713 | 2.747182046 | 0.426 | 0.474 | 0.059 | Glandular | SCGB3A1 |
| CA2 | 0.699 | 1.229858759 | 0.398 | 0.416 | 0.017 | Glandular | CA2 |
| EHF | 0.697 | 0.750765934 | 0.394 | 0.632 | 0.244 | Glandular | EHF |
| FAM3D | 0.696 | 0.989419199 | 0.392 | 0.482 | 0.091 | Glandular | FAM3D |
| LRRC26 | 0.691 | 1.081289988 | 0.382 | 0.402 | 0.02 | Glandular | LRRC26 |
| AQP5 | 0.688 | 0.80935635 | 0.376 | 0.538 | 0.162 | Glandular | AQP5 |
| PHLDA1 | 0.686 | 0.859152206 | 0.372 | 0.479 | 0.103 | Glandular | PHLDA1 |
| TMED3 | 0.685 | 0.8290259 | 0.37 | 0.511 | 0.148 | Glandular | TMED3 |
| PART1 | 0.681 | 0.976905707 | 0.362 | 0.397 | 0.035 | Glandular | PART1 |
| CST3 | 0.679 | 0.445780805 | 0.358 | 0.725 | 0.387 | Glandular | CST3 |
| PPP1R1B | 0.675 | 1.024761503 | 0.35 | 0.354 | 0.004 | Glandular | PPP1R1B |
| MSMB | 0.674 | 1.827667279 | 0.348 | 0.526 | 0.178 | Glandular | MSMB |
| CLDN10 | 0.672 | 0.903680482 | 0.344 | 0.406 | 0.061 | Glandular | CLDN10 |
| KIAA1324 | 0.671 | 0.875011509 | 0.342 | 0.415 | 0.074 | Glandular | KIAA1324 |
| FDCSP | 0.663 | 3.014683767 | 0.326 | 0.354 | 0.038 | Glandular | FDCSP |
| P4HB | 0.656 | 0.548227774 | 0.312 | 0.619 | 0.316 | Glandular | P4HB |
| PRR4 | 0.655 | 2.135335582 | 0.31 | 0.338 | 0.032 | Glandular | PRR4 |
| HP | 0.652 | 1.621721817 | 0.304 | 0.309 | 0.006 | Glandular | HP |
| MT-ND3 | 0.652 | 0.538785765 | 0.304 | 0.76 | 0.492 | Glandular | MT-ND3 |
| CAPS | 0.944 | 3.004338425 | 0.888 | 0.91 | 0.135 | Ciliated | CAPS |
| C9orf24 | 0.909 | 2.885810213 | 0.818 | 0.823 | 0.014 | Ciliated | C9orf24 |
| TSPAN11 | 0.907 | 1.995381768 | 0.814 | 0.908 | 0.242 | Ciliated | TSPAN1 |
| PIFO | 0.905 | 2.525174153 | 0.81 | 0.813 | 0.009 | Ciliated | PIFO |
| TPPP3 | 0.898 | 2.618650407 | 0.796 | 0.801 | 0.011 | Ciliated | TPPP3 |
| C20orf85 | 0.895 | 2.577997772 | 0.79 | 0.793 | 0.005 | Ciliated | C20orf85 |
| SNTN | 0.873 | 2.681568396 | 0.746 | 0.749 | 0.006 | Ciliated | SNTN |
| FAM183A | 0.865 | 2.305569681 | 0.73 | 0.733 | 0.006 | Ciliated | FAM183A |
| TUBB4B | 0.863 | 1.825335548 | 0.726 | 0.819 | 0.201 | Ciliated | TUBB4B |
| TUBA1A | 0.862 | 1.967069027 | 0.724 | 0.805 | 0.142 | Ciliated | TUBA1A |
| GSTA1 | 0.845 | 2.654446412 | 0.69 | 0.711 | 0.042 | Ciliated | GSTA1 |
| C11orf88 | 0.841 | 2.227327209 | 0.682 | 0.687 | 0.007 | Ciliated | C11orf88 |
| RSPH1 | 0.841 | 2.145088562 | 0.682 | 0.685 | 0.005 | Ciliated | RSPH1 |
| PRDX5 | 0.841 | 1.52896476 | 0.682 | 0.841 | 0.383 | Ciliated | PRDX5 |
| OMG | 0.839 | 2.541312303 | 0.678 | 0.683 | 0.008 | Ciliated | OMG |
| AGR3 | 0.838 | 2.262250865 | 0.676 | 0.693 | 0.026 | Ciliated | AGR3 |
| CAPSL | 0.838 | 2.152561508 | 0.676 | 0.679 | 0.003 | Ciliated | CAPSL |
| CIB1 | 0.838 | 1.588092054 | 0.676 | 0.807 | 0.245 | Ciliated | CIB1 |
| CCDC170 | 0.835 | 1.982934262 | 0.67 | 0.677 | 0.008 | Ciliated | CCDC170 |
| DYNLT1 | 0.833 | 1.697754258 | 0.666 | 0.765 | 0.181 | Ciliated | DYNLT1 |
| HSP90AA1 | 0.831 | 1.233988731 | 0.662 | 0.908 | 0.529 | Ciliated | HSP90AA1 |
| IFT57 | 0.829 | 1.809407937 | 0.658 | 0.715 | 0.088 | Ciliated | IFT57 |
| DNAH5 | 0.827 | 2.000492107 | 0.654 | 0.661 | 0.01 | Ciliated | DNAH5 |
| DYNLL1 | 0.824 | 1.323836698 | 0.648 | 0.839 | 0.32 | Ciliated | DYNLL1 |
| EZR1 | 0.822 | 1.07451605 | 0.644 | 0.91 | 0.515 | Ciliated | EZR |
| TMEM190 | 0.82 | 2.246164525 | 0.64 | 0.643 | 0.004 | Ciliated | TMEM190 |
| C1orf194 | 0.819 | 1.949286576 | 0.638 | 0.641 | 0.005 | Ciliated | C1orf194 |
| NUCB21 | 0.819 | 1.349294793 | 0.638 | 0.809 | 0.262 | Ciliated | NUCB2 |
| CALM1 | 0.819 | 1.074529846 | 0.638 | 0.9 | 0.557 | Ciliated | CALM1 |
| ATPIF1 | 0.818 | 1.370183159 | 0.636 | 0.825 | 0.366 | Ciliated | ATPIF1 |
| MORN2 | 0.816 | 1.821837128 | 0.632 | 0.657 | 0.035 | Ciliated | MORN2 |
| RP11-356K23.1 | 0.815 | 2.245200754 | 0.63 | 0.633 | 0.005 | Ciliated | RP11-356K23.1 |
| PSENEN | 0.815 | 1.787932278 | 0.63 | 0.697 | 0.107 | Ciliated | PSENEN |
| SPA17 | 0.812 | 1.910120724 | 0.624 | 0.635 | 0.013 | Ciliated | SPA17 |
| C9orf116 | 0.808 | 1.948263077 | 0.616 | 0.622 | 0.009 | Ciliated | C9orf116 |
| ZMYND10 | 0.803 | 1.820911729 | 0.606 | 0.608 | 0.004 | Ciliated | ZMYND10 |
| ROPN1L | 0.802 | 1.845602495 | 0.604 | 0.606 | 0.004 | Ciliated | ROPN1L |
| CETN2 | 0.801 | 1.703496691 | 0.602 | 0.639 | 0.049 | Ciliated | CETN2 |
| LRRIQ1 | 0.799 | 1.907757267 | 0.598 | 0.6 | 0.004 | Ciliated | LRRIQ1 |
| DNAH12 | 0.799 | 1.873790171 | 0.598 | 0.6 | 0.003 | Ciliated | DNAH12 |
| C5orf49 | 0.796 | 1.805426157 | 0.592 | 0.594 | 0.003 | Ciliated | C5orf49 |
| PLAC8 | 0.795 | 1.499632599 | 0.59 | 0.675 | 0.113 | Ciliated | PLAC8 |
| TMC5 | 0.791 | 1.670083838 | 0.582 | 0.629 | 0.067 | Ciliated | TMC5 |
| GSTP12 | 0.787 | 0.847373964 | 0.574 | 0.936 | 0.622 | Ciliated | GSTP1 |
| CCDC146 | 0.785 | 1.706153114 | 0.57 | 0.594 | 0.03 | Ciliated | CCDC146 |
| C1orf173 | 0.784 | 1.741885343 | 0.568 | 0.57 | 0.002 | Ciliated | C1orf173 |
| CALM2 | 0.783 | 0.890920229 | 0.566 | 0.892 | 0.557 | Ciliated | CALM2 |
| CYP4B1 | 0.782 | 1.482962727 | 0.564 | 0.679 | 0.151 | Ciliated | CYP4B1 |
| CHST9 | 0.78 | 1.384445369 | 0.56 | 0.663 | 0.136 | Ciliated | CHST9 |
| TCTEX1D4 | 0.779 | 1.856419769 | 0.558 | 0.562 | 0.005 | Ciliated | TCTEX1D4 |
| ARL3 | 0.777 | 1.356890107 | 0.554 | 0.677 | 0.168 | Ciliated | ARL3 |
| CD59 | 0.776 | 1.093742712 | 0.552 | 0.793 | 0.364 | Ciliated | CD59 |
| FAM216B | 0.774 | 1.720930876 | 0.548 | 0.55 | 0.002 | Ciliated | FAM216B |
| SPAG6 | 0.773 | 1.683149799 | 0.546 | 0.548 | 0.002 | Ciliated | SPAG6 |
| FAM154B | 0.771 | 1.64323743 | 0.542 | 0.546 | 0.004 | Ciliated | FAM154B |
| FAM81B | 0.769 | 1.682304435 | 0.538 | 0.54 | 0.002 | Ciliated | FAM81B |
| FAM229B | 0.769 | 1.55117494 | 0.538 | 0.564 | 0.031 | Ciliated | FAM229B |
| SMIM22 | 0.769 | 1.430699378 | 0.538 | 0.61 | 0.097 | Ciliated | SMIM22 |
| EFCAB1 | 0.768 | 1.612266032 | 0.536 | 0.54 | 0.006 | Ciliated | EFCAB1 |
| NQO1 | 0.765 | 1.375140039 | 0.53 | 0.602 | 0.087 | Ciliated | NQO1 |
| ABCA13 | 0.764 | 1.367717635 | 0.528 | 0.618 | 0.105 | Ciliated | ABCA13 |
| IK | 0.762 | 1.26928079 | 0.524 | 0.637 | 0.138 | Ciliated | IK |
| ARMC3 | 0.76 | 1.585921959 | 0.52 | 0.522 | 0.002 | Ciliated | ARMC3 |
| FOXJ1 | 0.757 | 1.534881131 | 0.514 | 0.52 | 0.007 | Ciliated | FOXJ1 |
| CDHR3 | 0.756 | 1.694771572 | 0.512 | 0.516 | 0.006 | Ciliated | CDHR3 |
| SCGB2A1 | 0.756 | 1.62559355 | 0.512 | 0.56 | 0.057 | Ciliated | SCGB2A1 |
| IQCG | 0.756 | 1.458665812 | 0.512 | 0.532 | 0.022 | Ciliated | IQCG |
| PRDX11 | 0.756 | 0.863725032 | 0.512 | 0.847 | 0.515 | Ciliated | PRDX1 |
| RRAD | 0.754 | 1.575138323 | 0.508 | 0.526 | 0.021 | Ciliated | RRAD |
| ANXA11 | 0.754 | 0.60639925 | 0.508 | 0.968 | 0.686 | Ciliated | ANXA1 |
| TSPAN19 | 0.753 | 1.698031523 | 0.506 | 0.508 | 0.003 | Ciliated | TSPAN19 |
| PCM1 | 0.753 | 1.153576174 | 0.506 | 0.671 | 0.201 | Ciliated | PCM1 |
| FAM92B | 0.752 | 1.499883316 | 0.504 | 0.514 | 0.012 | Ciliated | FAM92B |
| DYDC2 | 0.75 | 1.625667534 | 0.5 | 0.502 | 0.002 | Ciliated | DYDC2 |
| RSPH4A | 0.75 | 1.52691629 | 0.5 | 0.502 | 0.003 | Ciliated | RSPH4A |
| SLC44A4 | 0.75 | 1.369406443 | 0.5 | 0.558 | 0.069 | Ciliated | SLC44A4 |
| UFC1 | 0.75 | 1.203881897 | 0.5 | 0.62 | 0.146 | Ciliated | UFC1 |
| DNALI1 | 0.748 | 1.502798694 | 0.496 | 0.502 | 0.008 | Ciliated | DNALI1 |
| CKB | 0.748 | 1.407341072 | 0.496 | 0.558 | 0.072 | Ciliated | CKB |
| NME5 | 0.747 | 1.464483388 | 0.494 | 0.504 | 0.012 | Ciliated | NME5 |
| TEKT1 | 0.746 | 1.42660757 | 0.492 | 0.494 | 0.001 | Ciliated | TEKT1 |
| ODF3B | 0.745 | 1.484637651 | 0.49 | 0.524 | 0.041 | Ciliated | ODF3B |
| C9orf135 | 0.743 | 1.475080444 | 0.486 | 0.49 | 0.005 | Ciliated | C9orf135 |
| ALDH1A11 | 0.743 | 0.882747964 | 0.486 | 0.779 | 0.353 | Ciliated | ALDH1A1 |
| LGALS31 | 0.743 | 0.722765389 | 0.486 | 0.908 | 0.607 | Ciliated | LGALS3 |
| WDR78 | 0.742 | 1.449912204 | 0.484 | 0.492 | 0.009 | Ciliated | WDR78 |
| ODF2L | 0.741 | 1.274499598 | 0.482 | 0.56 | 0.085 | Ciliated | ODF2L |
| HSPH1 | 0.74 | 1.190057778 | 0.48 | 0.6 | 0.136 | Ciliated | HSPH1 |
| ALDH3B1 | 0.738 | 1.390763555 | 0.476 | 0.5 | 0.028 | Ciliated | ALDH3B1 |
| TSPAN6 | 0.737 | 1.251409749 | 0.474 | 0.556 | 0.101 | Ciliated | TSPAN6 |
| LRRC23 | 0.735 | 1.336955266 | 0.47 | 0.482 | 0.013 | Ciliated | LRRC23 |
| WDR52 | 0.733 | 1.44143623 | 0.466 | 0.49 | 0.026 | Ciliated | WDR52 |
| CTSS | 0.733 | 0.877617485 | 0.466 | 0.661 | 0.227 | Ciliated | CTSS |
| MS4A8 | 0.732 | 1.469324973 | 0.464 | 0.468 | 0.004 | Ciliated | MS4A8 |
| SPAG16 | 0.731 | 1.285169037 | 0.462 | 0.534 | 0.083 | Ciliated | SPAG16 |
| ENKUR | 0.73 | 1.4970937 | 0.46 | 0.462 | 0.002 | Ciliated | ENKUR |
| EFHC1 | 0.729 | 1.378123993 | 0.458 | 0.472 | 0.016 | Ciliated | EFHC1 |
| PSCA | 0.728 | 1.22313465 | 0.456 | 0.55 | 0.097 | Ciliated | PSCA |
| NUDC | 0.728 | 1.094024159 | 0.456 | 0.554 | 0.112 | Ciliated | NUDC |
| HMGN3 | 0.728 | 1.03433229 | 0.456 | 0.655 | 0.241 | Ciliated | HMGN3 |
| ZBBX | 0.725 | 1.378784079 | 0.45 | 0.452 | 0.001 | Ciliated | ZBBX |
| MLF1 | 0.725 | 1.285099149 | 0.45 | 0.488 | 0.041 | Ciliated | MLF1 |
| KIF21A | 0.725 | 1.126117706 | 0.45 | 0.57 | 0.132 | Ciliated | KIF21A |
| RSPH9 | 0.724 | 1.512068746 | 0.448 | 0.452 | 0.005 | Ciliated | RSPH9 |
| C10orf192 | 0.724 | 1.365640706 | 0.448 | 0.45 | 0.002 | Ciliated | C1orf192 |
| CCDC11 | 0.723 | 1.396708946 | 0.446 | 0.45 | 0.004 | Ciliated | CCDC11 |
| CCDC113 | 0.723 | 1.353684619 | 0.446 | 0.454 | 0.009 | Ciliated | CCDC113 |
| AK7 | 0.722 | 1.352975466 | 0.444 | 0.448 | 0.005 | Ciliated | AK7 |
| AKAP9 | 0.721 | 0.892065066 | 0.442 | 0.709 | 0.311 | Ciliated | AKAP9 |
| LDLRAD1 | 0.72 | 1.428232495 | 0.44 | 0.444 | 0.004 | Ciliated | LDLRAD1 |
| WDR54 | 0.72 | 1.344232534 | 0.44 | 0.458 | 0.02 | Ciliated | WDR54 |
| KIF9 | 0.72 | 1.295636355 | 0.44 | 0.46 | 0.022 | Ciliated | KIF9 |
| EFCAB10 | 0.72 | 1.29214429 | 0.44 | 0.442 | 0.002 | Ciliated | EFCAB10 |
| WDR96 | 0.719 | 1.405125932 | 0.438 | 0.44 | 0.002 | Ciliated | WDR96 |
| C12orf75 | 0.719 | 1.167737954 | 0.438 | 0.49 | 0.05 | Ciliated | C12orf75 |
| DYNLRB2 | 0.718 | 1.326144825 | 0.436 | 0.44 | 0.004 | Ciliated | DYNLRB2 |
| HSPB11 | 0.718 | 1.097722384 | 0.436 | 0.554 | 0.13 | Ciliated | HSPB11 |
| FXYD32 | 0.718 | 0.647562933 | 0.436 | 0.859 | 0.516 | Ciliated | FXYD3 |
| TSTD1 | 0.715 | 0.972101866 | 0.43 | 0.612 | 0.216 | Ciliated | TSTD1 |
| HSBP1 | 0.715 | 0.827138345 | 0.43 | 0.677 | 0.292 | Ciliated | HSBP1 |
| AKAP14 | 0.714 | 1.380566177 | 0.428 | 0.43 | 0.002 | Ciliated | AKAP14 |
| WDR86-AS1 | 0.713 | 1.389191661 | 0.426 | 0.438 | 0.011 | Ciliated | WDR86-AS1 |
| C10orf107 | 0.713 | 1.368131684 | 0.426 | 0.428 | 0.003 | Ciliated | C10orf107 |
| C11orf70 | 0.713 | 1.326059567 | 0.426 | 0.436 | 0.009 | Ciliated | C11orf70 |
| CES1 | 0.713 | 1.316099043 | 0.426 | 0.466 | 0.045 | Ciliated | CES1 |
| MNS1 | 0.713 | 1.30381609 | 0.426 | 0.432 | 0.006 | Ciliated | MNS1 |
| SPEF2 | 0.713 | 1.280773536 | 0.426 | 0.442 | 0.017 | Ciliated | SPEF2 |
| SPATA18 | 0.713 | 1.256887187 | 0.426 | 0.44 | 0.016 | Ciliated | SPATA18 |
| CCDC17 | 0.712 | 1.39309832 | 0.424 | 0.428 | 0.004 | Ciliated | CCDC17 |
| NPHP1 | 0.712 | 1.296322268 | 0.424 | 0.436 | 0.013 | Ciliated | NPHP1 |
| DPY30 | 0.712 | 1.01603303 | 0.424 | 0.554 | 0.146 | Ciliated | DPY30 |
| TAX1BP1 | 0.712 | 0.831194498 | 0.424 | 0.691 | 0.314 | Ciliated | TAX1BP1 |
| TCTEX1D2 | 0.711 | 1.310382676 | 0.422 | 0.444 | 0.025 | Ciliated | TCTEX1D2 |
| ARHGAP18 | 0.711 | 1.11310837 | 0.422 | 0.524 | 0.114 | Ciliated | ARHGAP18 |
| PPIL6 | 0.71 | 1.288461273 | 0.42 | 0.426 | 0.006 | Ciliated | PPIL6 |
| C14orf142 | 0.71 | 1.204639201 | 0.42 | 0.456 | 0.039 | Ciliated | C14orf142 |
| C21orf59 | 0.71 | 1.107703124 | 0.42 | 0.494 | 0.082 | Ciliated | C21orf59 |
| GSTA2 | 0.709 | 1.901688553 | 0.418 | 0.422 | 0.005 | Ciliated | GSTA2 |
| CCDC19 | 0.709 | 1.279825846 | 0.418 | 0.42 | 0.002 | Ciliated | CCDC19 |
| TMEM231 | 0.709 | 1.226775156 | 0.418 | 0.428 | 0.01 | Ciliated | TMEM231 |
| C6orf118 | 0.708 | 1.310162932 | 0.416 | 0.418 | 0.001 | Ciliated | C6orf118 |
| STOML3 | 0.708 | 1.261485584 | 0.416 | 0.418 | 0.001 | Ciliated | STOML3 |
| FANK1 | 0.708 | 1.181744444 | 0.416 | 0.422 | 0.005 | Ciliated | FANK1 |
| SEPW1 | 0.708 | 0.881854193 | 0.416 | 0.647 | 0.278 | Ciliated | SEPW1 |
| SPAG1 | 0.707 | 1.238657307 | 0.414 | 0.434 | 0.021 | Ciliated | SPAG1 |
| ALCAM1 | 0.707 | 0.773119228 | 0.414 | 0.673 | 0.272 | Ciliated | ALCAM |
| ANXA21 | 0.707 | 0.542412701 | 0.414 | 0.9 | 0.585 | Ciliated | ANXA2 |
| CSPP1 | 0.706 | 1.214843448 | 0.412 | 0.454 | 0.044 | Ciliated | CSPP1 |
| DHRS9 | 0.705 | 1.200130492 | 0.41 | 0.462 | 0.056 | Ciliated | DHRS9 |
| MRPS31 | 0.705 | 1.163304923 | 0.41 | 0.498 | 0.1 | Ciliated | MRPS31 |
| TSPAN3 | 0.703 | 0.803645249 | 0.406 | 0.641 | 0.262 | Ciliated | TSPAN3 |
| CYSTM1 | 0.702 | 0.936224152 | 0.404 | 0.536 | 0.145 | Ciliated | CYSTM1 |
| RP11-867G2.2 | 0.701 | 1.293159562 | 0.402 | 0.404 | 0.001 | Ciliated | RP11-867G2.2 |
| SRI | 0.7 | 0.90043817 | 0.4 | 0.596 | 0.227 | Ciliated | SRI |
| NEK10 | 0.699 | 1.333155928 | 0.398 | 0.4 | 0.002 | Ciliated | NEK10 |
| ANKUB1 | 0.699 | 1.283403857 | 0.398 | 0.402 | 0.004 | Ciliated | ANKUB1 |
| SYNE1 | 0.699 | 1.128785593 | 0.398 | 0.448 | 0.048 | Ciliated | SYNE1 |
| DPCD | 0.699 | 1.099524434 | 0.398 | 0.428 | 0.032 | Ciliated | DPCD |
| CATSPERD | 0.698 | 1.336630391 | 0.396 | 0.398 | 0.001 | Ciliated | CATSPERD |
| CCDC39 | 0.698 | 1.270822889 | 0.396 | 0.402 | 0.005 | Ciliated | CCDC39 |
| NWD1 | 0.698 | 1.146788139 | 0.396 | 0.43 | 0.034 | Ciliated | NWD1 |
| MORN5 | 0.697 | 1.32254625 | 0.394 | 0.396 | 0.002 | Ciliated | MORN5 |
| CD164 | 0.696 | 0.824239618 | 0.392 | 0.643 | 0.292 | Ciliated | CD164 |
| CLDN71 | 0.694 | 0.845920555 | 0.388 | 0.552 | 0.175 | Ciliated | CLDN7 |
| S100A62 | 0.693 | 0.484936939 | 0.386 | 0.958 | 0.818 | Ciliated | S100A6 |
| SAMHD1 | 0.692 | 0.852984577 | 0.384 | 0.488 | 0.101 | Ciliated | SAMHD1 |
| DNPH1 | 0.691 | 0.933679843 | 0.382 | 0.51 | 0.138 | Ciliated | DNPH1 |
| SPAG17 | 0.69 | 1.285853901 | 0.38 | 0.382 | 0.002 | Ciliated | SPAG17 |
| RP11-275114.4 | 0.69 | 1.224204271 | 0.38 | 0.384 | 0.005 | Ciliated | RP11-275114.4 |
| B9D1 | 0.69 | 1.187190724 | 0.38 | 0.408 | 0.031 | Ciliated | B9D1 |
| WDR66 | 0.69 | 1.174874571 | 0.38 | 0.39 | 0.008 | Ciliated | WDR66 |
| LRRC46 | 0.69 | 1.156605005 | 0.38 | 0.382 | 0.002 | Ciliated | LRRC46 |
| MAP3K19 | 0.689 | 1.197437938 | 0.378 | 0.38 | 0.001 | Ciliated | MAP3K19 |
| LRRC48 | 0.689 | 1.174140474 | 0.378 | 0.384 | 0.005 | Ciliated | LRRC48 |
| EFCAB2 | 0.688 | 1.115383906 | 0.376 | 0.4 | 0.023 | Ciliated | EFCAB2 |
| AGR21 | 0.688 | 0.383437939 | 0.376 | 0.785 | 0.43 | Ciliated | AGR2 |
| LINC00948 | 0.687 | 1.292941789 | 0.374 | 0.376 | 0.002 | Ciliated | LINC00948 |
| DNAAF1 | 0.687 | 1.22659294 | 0.374 | 0.404 | 0.029 | Ciliated | DNAAF1 |
| PROM1 | 0.687 | 1.131356434 | 0.374 | 0.422 | 0.054 | Ciliated | PROM1 |
| DNAJA4 | 0.686 | 1.168986227 | 0.372 | 0.394 | 0.023 | Ciliated | DNAJA4 |
| CDS1 | 0.686 | 1.107062845 | 0.372 | 0.402 | 0.032 | Ciliated | CDS1 |
| C9orf117 | 0.685 | 1.314988616 | 0.37 | 0.373 | 0.003 | Ciliated | C9orf117 |
| FHAD1 | 0.685 | 1.168624536 | 0.37 | 0.378 | 0.008 | Ciliated | FHAD1 |
| DNAH3 | 0.684 | 1.166841454 | 0.368 | 0.369 | 0.001 | Ciliated | DNAH3 |
| OSCP1 | 0.684 | 1.113746217 | 0.368 | 0.378 | 0.011 | Ciliated | OSCP1 |
| FAM174A | 0.684 | 1.047372787 | 0.368 | 0.43 | 0.066 | Ciliated | FAM174A |
| H2AFJ | 0.684 | 0.804188651 | 0.368 | 0.544 | 0.188 | Ciliated | H2AFJ |
| WFDC22 | 0.684 | 0.396942095 | 0.368 | 0.809 | 0.447 | Ciliated | WFDC2 |
| PIH1D2 | 0.683 | 1.165585634 | 0.366 | 0.371 | 0.005 | Ciliated | PIH1D2 |
| RABL5 | 0.683 | 1.049461426 | 0.366 | 0.396 | 0.031 | Ciliated | RABL5 |
| PERP2 | 0.683 | 0.520824494 | 0.366 | 0.833 | 0.492 | Ciliated | PERP |
| IFI27 | 0.682 | 0.68773636 | 0.364 | 0.637 | 0.281 | Ciliated | IFI27 |
| CCDC173 | 0.681 | 1.216677025 | 0.362 | 0.365 | 0.004 | Ciliated | CCDC173 |
| IGFBP2 | 0.681 | 0.853273841 | 0.362 | 0.452 | 0.087 | Ciliated | IGFBP2 |
| SAT11 | 0.681 | 0.443428773 | 0.362 | 0.89 | 0.641 | Ciliated | SAT1 |
| DTHD1 | 0.68 | 1.22059636 | 0.36 | 0.363 | 0.002 | Ciliated | DTHD1 |
| CCDC42B | 0.68 | 1.150766787 | 0.36 | 0.361 | 0.003 | Ciliated | CCDC42B |
| DNAH9 | 0.68 | 1.115435184 | 0.36 | 0.363 | 0.002 | Ciliated | DNAH9 |
| CCDC176 | 0.68 | 1.060916053 | 0.36 | 0.373 | 0.013 | Ciliated | CCDC176 |
| LZTFL1 | 0.679 | 1.014659554 | 0.358 | 0.398 | 0.041 | Ciliated | LZTFL1 |
| SOD1 | 0.679 | 0.591927932 | 0.358 | 0.711 | 0.389 | Ciliated | SOD1 |
| CLU | 0.679 | 0.520425061 | 0.358 | 0.667 | 0.321 | Ciliated | CLU |
| CCDC65 | 0.677 | 1.105012362 | 0.354 | 0.355 | 0.002 | Ciliated | CCDC65 |
| C11orf74 | 0.677 | 1.06702796 | 0.354 | 0.384 | 0.03 | Ciliated | C11orf74 |
| CTGF | 0.677 | 0.784265437 | 0.354 | 0.478 | 0.118 | Ciliated | CTGF |
| DRC1 | 0.676 | 1.148039329 | 0.352 | 0.353 | 0.001 | Ciliated | DRC1 |
| CASC1 | 0.676 | 1.066266015 | 0.352 | 0.355 | 0.004 | Ciliated | CASC1 |
| DSTN | 0.676 | 0.581644457 | 0.352 | 0.711 | 0.435 | Ciliated | DSTN |
| TRAF3IP1 | 0.675 | 1.015064688 | 0.35 | 0.384 | 0.033 | Ciliated | TRAF3IP1 |
| CCDC104 | 0.674 | 0.92849973 | 0.348 | 0.422 | 0.078 | Ciliated | CCDC104 |
| YWHAE | 0.674 | 0.584291104 | 0.348 | 0.671 | 0.363 | Ciliated | YWHAE |
| COX6A1 | 0.674 | 0.540187276 | 0.348 | 0.779 | 0.484 | Ciliated | COX6A1 |
| TMBIM6 | 0.674 | 0.517321509 | 0.348 | 0.797 | 0.541 | Ciliated | TMBIM6 |
| IFT172 | 0.673 | 0.961929075 | 0.346 | 0.394 | 0.049 | Ciliated | IFT172 |
| SLC27A2 | 0.673 | 0.884839793 | 0.346 | 0.422 | 0.074 | Ciliated | SLC27A2 |
| LRP11 | 0.671 | 0.946093409 | 0.342 | 0.39 | 0.05 | Ciliated | LRP11 |
| S100A111 | 0.671 | 0.50141302 | 0.342 | 0.807 | 0.55 | Ciliated | S100A11 |
| ALOX153 | 0.67 | 0.302739583 | 0.34 | 0.781 | 0.389 | Ciliated | ALOX15 |
| IFT43 | 0.669 | 0.931187744 | 0.338 | 0.394 | 0.057 | Ciliated | IFT43 |
| TXN1 | 0.669 | 0.49860407 | 0.338 | 0.785 | 0.496 | Ciliated | TXN |
| STK33 | 0.668 | 1.112434072 | 0.336 | 0.343 | 0.009 | Ciliated | STK33 |
| ARMC4 | 0.668 | 1.080981918 | 0.336 | 0.337 | 0.001 | Ciliated | ARMC4 |
| DZIP3 | 0.668 | 0.988105933 | 0.336 | 0.365 | 0.029 | Ciliated | DZIP3 |
| RAB11FIP1 | 0.668 | 0.679342215 | 0.336 | 0.5 | 0.16 | Ciliated | RAB11FIP1 |
| UBXN10 | 0.667 | 1.023889696 | 0.334 | 0.335 | 0.002 | Ciliated | UBXN10 |
| IFT81 | 0.667 | 0.880340237 | 0.334 | 0.388 | 0.052 | Ciliated | IFT81 |
| IGFBP7 | 0.666 | 0.439647955 | 0.332 | 0.633 | 0.292 | Ciliated | IGFBP7 |
| TTC18 | 0.665 | 1.157423704 | 0.33 | 0.341 | 0.012 | Ciliated | TTC18 |
| CYB5A | 0.665 | 0.703041399 | 0.33 | 0.524 | 0.205 | Ciliated | CYB5A |
| CAST | 0.665 | 0.590268329 | 0.33 | 0.647 | 0.334 | Ciliated | CAST |
| TMEM59 | 0.665 | 0.518694604 | 0.33 | 0.773 | 0.518 | Ciliated | TMEM59 |
| ELF31 | 0.665 | 0.439217997 | 0.33 | 0.739 | 0.394 | Ciliated | ELF3 |
| UBB | 0.665 | 0.423562879 | 0.33 | 0.882 | 0.749 | Ciliated | UBB |
| DNAH11 | 0.664 | 1.153895732 | 0.328 | 0.333 | 0.005 | Ciliated | DNAH11 |
| C7orf57 | 0.664 | 1.020705597 | 0.328 | 0.329 | 0.001 | Ciliated | C7orf57 |
| PTGES3 | 0.664 | 0.665214909 | 0.328 | 0.53 | 0.213 | Ciliated | PTGES3 |
| TTC29 | 0.663 | 1.096602043 | 0.326 | 0.327 | 0.001 | Ciliated | TTC29 |
| PPP1R42 | 0.663 | 1.066024124 | 0.326 | 0.327 | 0.002 | Ciliated | PPP1R42 |
| CLDN3 | 0.663 | 1.011881445 | 0.326 | 0.398 | 0.076 | Ciliated | CLDN3 |
| MUC16 | 0.663 | 0.916147826 | 0.326 | 0.392 | 0.064 | Ciliated | MUC16 |
| TUSC3 | 0.662 | 0.913759595 | 0.324 | 0.376 | 0.052 | Ciliated | TUSC3 |
| TCTN1 | 0.662 | 0.905314959 | 0.324 | 0.351 | 0.027 | Ciliated | TCTN1 |
| POLR2I | 0.662 | 0.788097995 | 0.324 | 0.44 | 0.118 | Ciliated | POLR2I |
| CCDC78 | 0.66 | 0.991789501 | 0.32 | 0.321 | 0.002 | Ciliated | CCDC78 |
| RUVBL2 | 0.66 | 0.970308174 | 0.32 | 0.373 | 0.057 | Ciliated | RUVBL2 |
| TNFAIP8L1 | 0.659 | 1.016226293 | 0.318 | 0.329 | 0.011 | Ciliated | TNFAIP8L1 |
| CC2D2A | 0.659 | 0.980737785 | 0.318 | 0.329 | 0.012 | Ciliated | CC2D2A |
| GDF15 | 0.659 | 0.78450862 | 0.318 | 0.424 | 0.102 | Ciliated | GDF15 |
| TAGLN2 | 0.659 | 0.563154137 | 0.318 | 0.643 | 0.344 | Ciliated | TAGLN2 |
| CDHR4 | 0.658 | 1.055016719 | 0.316 | 0.317 | 0.002 | Ciliated | CDHR4 |
| DNAL1 | 0.658 | 0.964059769 | 0.316 | 0.337 | 0.021 | Ciliated | DNAL1 |
| ECT2L | 0.657 | 1.047412221 | 0.314 | 0.315 | 0.002 | Ciliated | ECT2L |
| RUVBL1 | 0.657 | 0.855540355 | 0.314 | 0.353 | 0.039 | Ciliated | RUVBL1 |
| SYAP1 | 0.657 | 0.698489456 | 0.314 | 0.502 | 0.194 | Ciliated | SYAP1 |
| METTL7A | 0.657 | 0.640005993 | 0.314 | 0.55 | 0.249 | Ciliated | METTL7A |
| DNAH7 | 0.655 | 1.121023481 | 0.31 | 0.315 | 0.006 | Ciliated | DNAH7 |
| IQCD | 0.655 | 0.930324085 | 0.31 | 0.313 | 0.003 | Ciliated | IQCD |
| NDUFB1 | 0.655 | 0.551282587 | 0.31 | 0.649 | 0.377 | Ciliated | NDUFB1 |
| UBL5 | 0.655 | 0.526083258 | 0.31 | 0.715 | 0.471 | Ciliated | UBL5 |
| RP4-666F24.3 | 0.654 | 1.016307257 | 0.308 | 0.309 | 0.001 | Ciliated | RP4-666F24.3 |
| C9orf9 | 0.654 | 0.92195711 | 0.308 | 0.315 | 0.007 | Ciliated | C9orf9 |
| C21orf58 | 0.653 | 1.049751919 | 0.306 | 0.313 | 0.008 | Ciliated | C21orf58 |
| ANKRD66 | 0.653 | 1.048555371 | 0.306 | 0.307 | 0 | Ciliated | ANKRD66 |
| EPCAM | 0.653 | 0.677163693 | 0.306 | 0.448 | 0.141 | Ciliated | EPCAM |
| PCDP1 | 0.652 | 0.984790706 | 0.304 | 0.305 | 0.002 | Ciliated | PCDP1 |
| CMPK1 | 0.651 | 0.652227114 | 0.302 | 0.508 | 0.222 | Ciliated | CMPK1 |
| TMEM14B | 0.651 | 0.639804416 | 0.302 | 0.466 | 0.164 | Ciliated | TMEM14B |
| MORF4L2 | 0.651 | 0.572272238 | 0.302 | 0.5 | 0.197 | Ciliated | MORF4L2 |
| MT-RNR1 | 0.651 | 0.500992493 | 0.302 | 0.964 | 0.874 | Ciliated | MT-RNR1 |
| DCN | 0.954 | 3.664226439 | 0.908 | 0.921 | 0.077 | Fibroblast | DCN |
| COL1A2 | 0.901 | 2.956404379 | 0.802 | 0.817 | 0.041 | Fibroblast | COL1A2 |
| LUM | 0.893 | 3.177569902 | 0.786 | 0.807 | 0.063 | Fibroblast | LUM |
| COL3A1 | 0.883 | 3.110452512 | 0.766 | 0.784 | 0.044 | Fibroblast | COL3A1 |
| MGP | 0.875 | 3.021700711 | 0.75 | 0.785 | 0.092 | Fibroblast | MGP |
| LGALS1 | 0.869 | 2.151012324 | 0.738 | 0.818 | 0.179 | Fibroblast | LGALS1 |
| CALD1 | 0.846 | 2.319578137 | 0.692 | 0.734 | 0.084 | Fibroblast | CALD1 |
| IGFBP71 | 0.838 | 1.861702438 | 0.676 | 0.804 | 0.249 | Fibroblast | IGFBP7 |
| FBLN1 | 0.835 | 2.504229506 | 0.67 | 0.696 | 0.056 | Fibroblast | FBLN1 |
| CPE | 0.834 | 2.448210765 | 0.668 | 0.693 | 0.045 | Fibroblast | CPE |
| SPARC | 0.819 | 2.096929966 | 0.638 | 0.696 | 0.087 | Fibroblast | SPARC |
| VIM2 | 0.813 | 1.324691008 | 0.626 | 0.83 | 0.303 | Fibroblast | VIM |
| POSTN3 | 0.791 | 1.551239159 | 0.582 | 0.822 | 0.415 | Fibroblast | POSTN |
| IFITM3 | 0.781 | 1.32904463 | 0.562 | 0.756 | 0.325 | Fibroblast | IFITM3 |
| SFRP1 | 0.776 | 2.270240988 | 0.552 | 0.58 | 0.047 | Fibroblast | SFRP1 |
| SFRP2 | 0.773 | 2.533081437 | 0.546 | 0.56 | 0.023 | Fibroblast | SFRP2 |
| C1S | 0.77 | 2.101462597 | 0.54 | 0.561 | 0.034 | Fibroblast | C1S |
| COL1A1 | 0.765 | 2.310167816 | 0.53 | 0.546 | 0.025 | Fibroblast | COL1A1 |
| SERPING1 | 0.761 | 1.947212662 | 0.522 | 0.553 | 0.049 | Fibroblast | SERPING1 |
| AEBP1 | 0.758 | 2.139687771 | 0.516 | 0.535 | 0.027 | Fibroblast | AEBP1 |
| PCOLCE | 0.752 | 1.979823826 | 0.504 | 0.52 | 0.023 | Fibroblast | PCOLCE |
| TAGLN | 0.741 | 2.278648097 | 0.482 | 0.513 | 0.043 | Fibroblast | TAGLN |
| C1R | 0.74 | 1.854135357 | 0.48 | 0.513 | 0.047 | Fibroblast | C1R |
| SEPP1 | 0.74 | 1.412080819 | 0.48 | 0.65 | 0.276 | Fibroblast | SEPP1 |
| PPAP2B | 0.726 | 1.724337273 | 0.452 | 0.52 | 0.103 | Fibroblast | PPAP2B |
| CRABP2 | 0.725 | 1.98189539 | 0.45 | 0.466 | 0.021 | Fibroblast | CRABP2 |
| TPM2 | 0.725 | 1.937902606 | 0.45 | 0.482 | 0.046 | Fibroblast | TPM2 |
| IGFBP6 | 0.722 | 2.018823128 | 0.444 | 0.462 | 0.027 | Fibroblast | IGFBP6 |
| THY1 | 0.721 | 1.931282203 | 0.442 | 0.454 | 0.016 | Fibroblast | THY1 |
| CDH11 | 0.712 | 1.829191098 | 0.424 | 0.443 | 0.028 | Fibroblast | CDH11 |
| CXCL14 | 0.711 | 2.327803164 | 0.422 | 0.44 | 0.026 | Fibroblast | CXCL14 |
| FGF7 | 0.711 | 1.983574452 | 0.422 | 0.43 | 0.01 | Fibroblast | FGF7 |
| SELM | 0.711 | 1.249225801 | 0.422 | 0.599 | 0.276 | Fibroblast | SELM |
| TMSB4X2 | 0.711 | 0.579398488 | 0.422 | 0.873 | 0.672 | Fibroblast | TMSB4X |
| RARRES2 | 0.71 | 1.746628419 | 0.42 | 0.441 | 0.03 | Fibroblast | RARRES2 |
| VCAN | 0.708 | 1.770951104 | 0.416 | 0.446 | 0.036 | Fibroblast | VCAN |
| PRRX1 | 0.708 | 1.750819728 | 0.416 | 0.43 | 0.018 | Fibroblast | PRRX1 |
| CLDN11 | 0.707 | 1.900723255 | 0.414 | 0.426 | 0.017 | Fibroblast | CLDN11 |
| TPM1 | 0.707 | 1.726675629 | 0.414 | 0.491 | 0.114 | Fibroblast | TPM1 |
| NNMT | 0.703 | 1.840571739 | 0.406 | 0.443 | 0.047 | Fibroblast | NNMT |
| IGFBP4 | 0.697 | 1.541049782 | 0.394 | 0.451 | 0.074 | Fibroblast | IGFBP4 |
| BGN | 0.694 | 1.633620282 | 0.388 | 0.4 | 0.013 | Fibroblast | BGN |
| LAPTM4A | 0.693 | 0.990386779 | 0.386 | 0.625 | 0.368 | Fibroblast | LAPTM4A |
| PDGFRA | 0.692 | 1.711725475 | 0.384 | 0.392 | 0.009 | Fibroblast | PDGFRA |
| COL6A2 | 0.691 | 1.748221842 | 0.382 | 0.396 | 0.018 | Fibroblast | COL6A2 |
| MXRA8 | 0.691 | 1.662700365 | 0.382 | 0.391 | 0.01 | Fibroblast | MXRA8 |
| LIMA1 | 0.69 | 1.169453194 | 0.38 | 0.564 | 0.273 | Fibroblast | LIMA1 |
| S100A63 | 0.686 | 0.462950462 | 0.372 | 0.948 | 0.808 | Fibroblast | S100A6 |
| APOD | 0.685 | 1.827669597 | 0.37 | 0.41 | 0.054 | Fibroblast | APOD |
| FSTL1 | 0.681 | 1.482852645 | 0.362 | 0.411 | 0.062 | Fibroblast | FSTL1 |
| LAMP5 | 0.68 | 1.705950601 | 0.36 | 0.374 | 0.017 | Fibroblast | LAMP5 |
| NBL1 | 0.677 | 1.547509772 | 0.354 | 0.401 | 0.061 | Fibroblast | NBL1 |
| THBS1 | 0.675 | 1.500106103 | 0.35 | 0.412 | 0.074 | Fibroblast | THBS1 |
| EID1 | 0.675 | 0.908943223 | 0.35 | 0.625 | 0.413 | Fibroblast | EID1 |
| IL6ST | 0.671 | 1.044758173 | 0.342 | 0.535 | 0.263 | Fibroblast | IL6ST |
| KCNE4 | 0.668 | 1.811316366 | 0.336 | 0.344 | 0.008 | Fibroblast | KCNE4 |
| IGF2 | 0.667 | 1.711402709 | 0.334 | 0.342 | 0.008 | Fibroblast | IGF2 |
| COL6A1 | 0.667 | 1.516877531 | 0.334 | 0.376 | 0.053 | Fibroblast | COL6A1 |
| CCL2 | 0.666 | 1.821838159 | 0.332 | 0.38 | 0.059 | Fibroblast | CCL2 |
| MFAP4 | 0.666 | 1.543146102 | 0.332 | 0.343 | 0.012 | Fibroblast | MFAP4 |
| COL15A1 | 0.666 | 1.449124768 | 0.332 | 0.365 | 0.038 | Fibroblast | COL15A1 |
| ITGBL1 | 0.665 | 1.636117859 | 0.33 | 0.335 | 0.006 | Fibroblast | ITGBL1 |
| COL8A1 | 0.662 | 1.670546037 | 0.324 | 0.331 | 0.009 | Fibroblast | COL8A1 |
| GLIPR1 | 0.661 | 1.457087277 | 0.322 | 0.369 | 0.056 | Fibroblast | GLIPR1 |
| TIMP3 | 0.659 | 1.397105976 | 0.318 | 0.369 | 0.06 | Fibroblast | TIMP3 |
| RGS5 | 0.658 | 2.141182557 | 0.316 | 0.357 | 0.052 | Fibroblast | RGS5 |
| MYL9 | 0.657 | 1.352225212 | 0.314 | 0.382 | 0.084 | Fibroblast | MYL9 |
| ITGB1 | 0.656 | 1.04438448 | 0.312 | 0.469 | 0.211 | Fibroblast | ITGB1 |
| APP | 0.656 | 1.012040303 | 0.312 | 0.521 | 0.284 | Fibroblast | APP |
| RARRES1 | 0.654 | 1.293952578 | 0.308 | 0.456 | 0.177 | Fibroblast | RARRES1 |
| SERPINF11 | 0.654 | 0.958611643 | 0.308 | 0.479 | 0.209 | Fibroblast | SERPINF1 |
| TGM2 | 0.651 | 1.42835406 | 0.302 | 0.336 | 0.042 | Fibroblast | TGM2 |
| CLU1 | 0.651 | 0.897011169 | 0.302 | 0.54 | 0.309 | Fibroblast | CLU |
| SPARCL1 | 0.927 | 2.639794324 | 0.854 | 0.89 | 0.128 | Endothelial | SPARCL1 |
| VIM3 | 0.879 | 1.600228964 | 0.758 | 0.917 | 0.316 | Endothelial | VIM |
| HLA-E | 0.87 | 1.34262652 | 0.74 | 0.925 | 0.588 | Endothelial | HLA-E |
| GNG11 | 0.86 | 2.242598011 | 0.72 | 0.765 | 0.077 | Endothelial | GNG11 |
| A2M | 0.839 | 2.144569958 | 0.678 | 0.721 | 0.063 | Endothelial | A2M |
| TMSB101 | 0.837 | 0.955903075 | 0.674 | 0.969 | 0.808 | Endothelial | TMSB10 |
| IFI271 | 0.824 | 1.57708082 | 0.648 | 0.798 | 0.256 | Endothelial | IFI27 |
| IGFBP72 | 0.817 | 1.363015878 | 0.634 | 0.815 | 0.267 | Endothelial | IGFBP7 |
| IFITM31 | 0.812 | 1.283212988 | 0.624 | 0.827 | 0.335 | Endothelial | IFITM3 |
| CD741 | 0.809 | 0.938321276 | 0.618 | 0.859 | 0.425 | Endothelial | CD74 |
| CLDN5 | 0.794 | 2.432750719 | 0.588 | 0.596 | 0.014 | Endothelial | CLDN5 |
| ELTD1 | 0.789 | 2.017121957 | 0.578 | 0.587 | 0.01 | Endothelial | ELTD1 |
| TMSB4X3 | 0.774 | 0.690088145 | 0.548 | 0.946 | 0.674 | Endothelial | TMSB4X |
| DARC | 0.769 | 2.391764767 | 0.538 | 0.548 | 0.018 | Endothelial | DARC |
| EMCN | 0.768 | 2.008778316 | 0.536 | 0.542 | 0.008 | Endothelial | EMCN |
| TM4SF1 | 0.761 | 1.462365672 | 0.522 | 0.662 | 0.204 | Endothelial | TM4SF1 |
| SPARC1 | 0.754 | 1.306249621 | 0.508 | 0.619 | 0.113 | Endothelial | SPARC |
| PTRF | 0.745 | 1.35871148 | 0.49 | 0.591 | 0.116 | Endothelial | PTRF |
| VWF | 0.731 | 1.879276683 | 0.462 | 0.474 | 0.014 | Endothelial | VWF |
| GIMAP7 | 0.726 | 1.612614182 | 0.452 | 0.477 | 0.024 | Endothelial | GIMAP7 |
| IFITM2 | 0.725 | 1.121019115 | 0.45 | 0.606 | 0.188 | Endothelial | IFITM2 |
| PLVAP | 0.722 | 1.775407995 | 0.444 | 0.449 | 0.006 | Endothelial | PLVAP |
| RPS231 | 0.722 | 0.564985387 | 0.444 | 0.927 | 0.827 | Endothelial | RPS23 |
| RPL33 | 0.721 | 0.574486036 | 0.442 | 0.936 | 0.833 | Endothelial | RPL3 |
| ECSCR | 0.719 | 1.611564103 | 0.438 | 0.444 | 0.006 | Endothelial | ECSCR |
| RPL321 | 0.719 | 0.523983371 | 0.438 | 0.941 | 0.863 | Endothelial | RPL32 |
| EMP1 | 0.717 | 1.315314552 | 0.434 | 0.56 | 0.153 | Endothelial | EMP1 |
| HLA-DRB1 | 0.716 | 0.643508106 | 0.432 | 0.594 | 0.179 | Endothelial | HLA-DRB1 |
| RAMP2 | 0.714 | 1.57933047 | 0.428 | 0.437 | 0.01 | Endothelial | RAMP2 |
| CALCRL | 0.714 | 1.544415356 | 0.428 | 0.457 | 0.031 | Endothelial | CALCRL |
| PTMA1 | 0.71 | 0.59823819 | 0.42 | 0.88 | 0.687 | Endothelial | PTMA |
| HLA-DRA | 0.709 | 0.361572144 | 0.418 | 0.621 | 0.237 | Endothelial | HLA-DRA |
| RPL311 | 0.708 | 0.439633753 | 0.416 | 0.973 | 0.92 | Endothelial | RPL31 |
| ESAM | 0.706 | 1.543211372 | 0.412 | 0.418 | 0.008 | Endothelial | ESAM |
| ID3 | 0.706 | 1.046645794 | 0.412 | 0.615 | 0.263 | Endothelial | ID3 |
| APOLD1 | 0.703 | 1.735046997 | 0.406 | 0.416 | 0.012 | Endothelial | APOLD1 |
| FKBP1A | 0.698 | 1.264154422 | 0.396 | 0.498 | 0.132 | Endothelial | FKBP1A |
| ADAMTS1 | 0.697 | 1.575767844 | 0.394 | 0.442 | 0.05 | Endothelial | ADAMTS1 |
| ADIRF | 0.694 | 1.072540948 | 0.388 | 0.623 | 0.328 | Endothelial | ADIRF |
| RPS27A1 | 0.693 | 0.402404818 | 0.386 | 0.958 | 0.898 | Endothelial | RPS27A |
| RAMP3 | 0.691 | 1.559843994 | 0.382 | 0.388 | 0.007 | Endothelial | RAMP3 |
| RPS15A1 | 0.691 | 0.431336114 | 0.382 | 0.949 | 0.851 | Endothelial | RPS15A |
| EGFL7 | 0.689 | 1.491656767 | 0.378 | 0.393 | 0.018 | Endothelial | EGFL7 |
| HLA-B | 0.689 | 0.460865321 | 0.378 | 0.919 | 0.816 | Endothelial | HLA-B |
| RPS13 | 0.686 | 0.399249269 | 0.372 | 0.954 | 0.885 | Endothelial | RPS13 |
| IL331 | 0.685 | 1.02660227 | 0.37 | 0.549 | 0.213 | Endothelial | IL33 |
| RPS31 | 0.684 | 0.446136032 | 0.368 | 0.907 | 0.802 | Endothelial | RPS3 |
| LIFR | 0.682 | 1.409478186 | 0.364 | 0.395 | 0.039 | Endothelial | LIFR |
| CAV1 | 0.682 | 1.321241408 | 0.364 | 0.421 | 0.063 | Endothelial | CAV1 |
| NPDC1 | 0.682 | 1.308504118 | 0.364 | 0.42 | 0.069 | Endothelial | NPDC1 |
| CD34 | 0.681 | 1.394952772 | 0.362 | 0.375 | 0.015 | Endothelial | CD34 |
| AC011526.1 | 0.68 | 1.401187771 | 0.36 | 0.366 | 0.005 | Endothelial | AC011526.1 |
| RPS9 | 0.676 | 0.453059926 | 0.352 | 0.894 | 0.794 | Endothelial | RPS9 |
| NOSTRIN | 0.675 | 1.440052876 | 0.35 | 0.36 | 0.011 | Endothelial | NOSTRIN |
| RPL24 | 0.675 | 0.466895031 | 0.35 | 0.867 | 0.739 | Endothelial | RPL24 |
| IL6ST1 | 0.674 | 0.850324428 | 0.348 | 0.575 | 0.27 | Endothelial | IL6ST |
| CYYR1 | 0.672 | 1.251618825 | 0.344 | 0.363 | 0.021 | Endothelial | CYYR1 |
| CRIP2 | 0.671 | 1.23278709 | 0.342 | 0.392 | 0.061 | Endothelial | CRIP2 |
| RDX | 0.671 | 1.128671963 | 0.342 | 0.464 | 0.149 | Endothelial | RDX |
| RPL5 | 0.671 | 0.458752835 | 0.342 | 0.885 | 0.781 | Endothelial | RPL5 |
| JAM2 | 0.67 | 1.314431376 | 0.34 | 0.353 | 0.013 | Endothelial | JAM2 |
| TGFBR2 | 0.668 | 1.204193722 | 0.336 | 0.389 | 0.059 | Endothelial | TGFBR2 |
| STOM | 0.666 | 0.985719999 | 0.332 | 0.454 | 0.136 | Endothelial | STOM |
| TPM3 | 0.666 | 0.765042741 | 0.332 | 0.566 | 0.278 | Endothelial | TPM3 |
| TχNIP | 0.665 | 0.643459581 | 0.33 | 0.728 | 0.508 | Endothelial | TXNIP |
| HLA-DPA1 | 0.664 | 0.48303852 | 0.328 | 0.451 | 0.123 | Endothelial | HLA-DPA1 |
| RPL261 | 0.663 | 0.430661446 | 0.326 | 0.878 | 0.782 | Endothelial | RPL26 |
| TSPAN7 | 0.662 | 1.386845693 | 0.324 | 0.331 | 0.007 | Endothelial | TSPAN7 |
| ENG | 0.662 | 1.252565568 | 0.324 | 0.346 | 0.023 | Endothelial | ENG |
| SPRY1 | 0.66 | 1.241822164 | 0.32 | 0.393 | 0.084 | Endothelial | SPRY1 |
| EEF1A1 | 0.658 | 0.552725953 | 0.316 | 0.773 | 0.621 | Endothelial | EEF1A1 |
| PALMD | 0.657 | 1.233339207 | 0.314 | 0.366 | 0.061 | Endothelial | PALMD |
| SPTBN1 | 0.657 | 0.995782043 | 0.314 | 0.423 | 0.126 | Endothelial | SPTBN1 |
| RPL9 | 0.657 | 0.588417285 | 0.314 | 0.74 | 0.558 | Endothelial | RPL9 |
| CD93 | 0.655 | 1.205461496 | 0.31 | 0.322 | 0.013 | Endothelial | CD93 |
| ELK3 | 0.654 | 1.099511055 | 0.308 | 0.358 | 0.054 | Endothelial | ELK3 |
| SOCS3 | 0.654 | 0.700528859 | 0.308 | 0.58 | 0.315 | Endothelial | SOCS3 |
| MYL12A | 0.653 | 0.664520921 | 0.306 | 0.64 | 0.412 | Endothelial | MYL12A |
| RPL19 | 0.653 | 0.307991654 | 0.306 | 0.951 | 0.898 | Endothelial | RPL19 |
| SELE | 0.652 | 2.014547668 | 0.304 | 0.31 | 0.007 | Endothelial | SELE |
| KCTD12 | 0.652 | 1.153063451 | 0.304 | 0.343 | 0.041 | Endothelial | KCTD12 |
| RPL121 | 0.652 | 0.38389977 | 0.304 | 0.885 | 0.786 | Endothelial | RPL12 |
| HLA-DRB5 | 0.651 | 0.821245039 | 0.302 | 0.389 | 0.09 | Endothelial | HLA-DRB5 |
| RPL101 | 0.651 | 0.538017238 | 0.302 | 0.775 | 0.622 | Endothelial | RPL10 |
| IGJ2 | 0.97 | 4.084220098 | 0.94 | 0.976 | 0.401 | PlasmaCell | IGJ |
| SSR41 | 0.938 | 2.168855212 | 0.876 | 0.943 | 0.508 | PlasmaCell | SSR4 |
| MZB1 | 0.896 | 2.7814926 | 0.792 | 0.812 | 0.05 | PlasmaCell | MZB1 |
| IGHA1 | 0.878 | 3.756591266 | 0.756 | 0.925 | 0.49 | PlasmaCell | IGHA1 |
| SEC11C | 0.84 | 1.814937713 | 0.68 | 0.785 | 0.247 | PlasmaCell | SEC11C |
| HSP90B1 | 0.83 | 1.48372228 | 0.66 | 0.864 | 0.537 | PlasmaCell | HSP90B1 |
| ENAM | 0.828 | 3.079234646 | 0.656 | 0.67 | 0.025 | PlasmaCell | ENAM |
| IGHG1 | 0.81 | 4.368283757 | 0.62 | 0.684 | 0.124 | PlasmaCell | IGHG1 |
| IGHA2 | 0.806 | 3.673825536 | 0.612 | 0.703 | 0.161 | PlasmaCell | IGHA2 |
| IGHG4 | 0.795 | 4.401457544 | 0.59 | 0.677 | 0.164 | PlasmaCell | IGHG4 |
| IGHG3 | 0.795 | 4.302923806 | 0.59 | 0.669 | 0.144 | PlasmaCell | IGHG3 |
| HERPUD1 | 0.785 | 1.516025336 | 0.57 | 0.722 | 0.27 | PlasmaCell | HERPUD1 |
| DERL3 | 0.783 | 2.010532408 | 0.566 | 0.585 | 0.026 | PlasmaCell | DERL3 |
| PRDX4 | 0.776 | 1.52963904 | 0.552 | 0.677 | 0.216 | PlasmaCell | PRDX4 |
| IGHG2 | 0.768 | 4.0583124 | 0.536 | 0.563 | 0.046 | PlasmaCell | IGHG2 |
| FKBP11 | 0.768 | 1.735733039 | 0.536 | 0.58 | 0.066 | PlasmaCell | FKBP11 |
| IGKC | 0.767 | 3.876579392 | 0.534 | 0.618 | 0.153 | PlasmaCell | IGKC |
| AC096579.7 | 0.763 | 3.386232865 | 0.526 | 0.54 | 0.017 | PlasmaCell | AC096579.7 |
| SPCS3 | 0.763 | 1.305504503 | 0.526 | 0.705 | 0.291 | PlasmaCell | SPCS3 |
| RGS1 | 0.748 | 1.415960223 | 0.496 | 0.609 | 0.121 | PlasmaCell | RGS1 |
| TSC22D3 | 0.737 | 1.138031558 | 0.474 | 0.712 | 0.348 | PlasmaCell | TSC22D3 |
| SLAMF7 | 0.724 | 1.546134265 | 0.448 | 0.462 | 0.015 | PlasmaCell | SLAMF7 |
| FAM46C | 0.723 | 1.393140859 | 0.446 | 0.518 | 0.091 | PlasmaCell | FAM46C |
| SSR3 | 0.723 | 1.170089703 | 0.446 | 0.622 | 0.252 | PlasmaCell | SSR3 |
| PIM2 | 0.712 | 1.528537291 | 0.424 | 0.454 | 0.034 | PlasmaCell | PIM2 |
| RNA28S5 | 0.707 | 0.606796234 | 0.414 | 0.885 | 0.776 | PlasmaCell | RNA28S5 |
| CD79A | 0.695 | 1.448954462 | 0.39 | 0.4 | 0.011 | PlasmaCell | CD79A |
| XBP13 | 0.695 | 0.785903609 | 0.39 | 0.723 | 0.411 | PlasmaCell | XBP1 |
| ITM2C | 0.69 | 1.230980644 | 0.38 | 0.468 | 0.111 | PlasmaCell | ITM2C |
| IGLC7 | 0.689 | 3.615271123 | 0.378 | 0.41 | 0.044 | PlasmaCell | IGLC7 |
| SEL1L | 0.678 | 1.137420631 | 0.356 | 0.455 | 0.118 | PlasmaCell | SEL1L |
| IGLL1 | 0.677 | 3.193768395 | 0.354 | 0.367 | 0.017 | PlasmaCell | IGLL1 |
| FCRL5 | 0.676 | 1.349224306 | 0.352 | 0.358 | 0.007 | PlasmaCell | FCRL5 |
| PRDM1 | 0.676 | 1.3179458 | 0.352 | 0.386 | 0.037 | PlasmaCell | PRDM1 |
| TRAM1 | 0.676 | 0.857202899 | 0.352 | 0.594 | 0.307 | PlasmaCell | TRAM1 |
| UBE2J1 | 0.674 | 1.149212986 | 0.348 | 0.444 | 0.116 | PlasmaCell | UBE2J1 |
| RRBP1 | 0.673 | 0.967773784 | 0.346 | 0.557 | 0.269 | PlasmaCell | RRBP1 |
| SUB1 | 0.673 | 0.869953646 | 0.346 | 0.583 | 0.298 | PlasmaCell | SUB1 |
| SPCS1 | 0.672 | 0.848098235 | 0.344 | 0.585 | 0.311 | PlasmaCell | SPCS1 |
| CCND2 | 0.671 | 1.495829807 | 0.342 | 0.397 | 0.065 | PlasmaCell | CCND2 |
| IGLC3 | 0.667 | 3.690287734 | 0.334 | 0.352 | 0.025 | PlasmaCell | IGLC3 |
| ERLEC1 | 0.667 | 0.907789033 | 0.334 | 0.512 | 0.213 | PlasmaCell | ERLEC1 |
| FKBP2 | 0.665 | 0.930133641 | 0.33 | 0.495 | 0.198 | PlasmaCell | FKBP2 |
| SPCS2 | 0.659 | 0.934230056 | 0.318 | 0.445 | 0.146 | PlasmaCell | SPCS2 |
| SELK | 0.656 | 0.717525057 | 0.312 | 0.576 | 0.314 | PlasmaCell | SELK |
| IGKV3-20 | 0.654 | 3.146568263 | 0.308 | 0.317 | 0.011 | PlasmaCell | IGKV3-20 |
| ISG20 | 0.654 | 1.017553056 | 0.308 | 0.388 | 0.088 | PlasmaCell | ISG20 |
| CYBA | 0.653 | 0.864156646 | 0.306 | 0.445 | 0.153 | PlasmaCell | CYBA |
| C19orf10 | 0.651 | 0.889006411 | 0.302 | 0.485 | 0.219 | PlasmaCell | C19orf10 |
| TMSB4X4 | 0.883 | 1.378073792 | 0.766 | 0.966 | 0.678 | TCell | TMSB4X |
| RPS29 | 0.86 | 1.109312061 | 0.72 | 0.977 | 0.912 | TCell | RPS29 |
| RPS27A2 | 0.859 | 1.005615059 | 0.718 | 0.98 | 0.898 | TCell | RPS27A |
| CD52 | 0.847 | 2.42082371 | 0.694 | 0.741 | 0.092 | TCell | CD52 |
| RPS15A2 | 0.835 | 1.016095334 | 0.67 | 0.958 | 0.852 | TCell | RPS15A |
| RPS27 | 0.817 | 1.632784817 | 0.634 | 0.84 | 0.559 | TCell | RPS27 |
| CXCR4 | 0.805 | 2.229205328 | 0.61 | 0.669 | 0.119 | TCell | CXCR4 |
| RPS252 | 0.805 | 1.034505157 | 0.61 | 0.919 | 0.782 | TCell | RPS25 |
| SRGN1 | 0.795 | 1.314836596 | 0.59 | 0.78 | 0.295 | TCell | SRGN |
| PTPRC | 0.788 | 2.227320512 | 0.576 | 0.619 | 0.071 | TCell | PTPRC |
| TRBC2 | 0.777 | 2.49597531 | 0.554 | 0.566 | 0.024 | TCell | TRBC2 |
| RPL13A2 | 0.759 | 0.752793694 | 0.518 | 0.951 | 0.883 | TCell | RPL13A |
| RPS202 | 0.758 | 0.740564088 | 0.516 | 0.94 | 0.872 | TCell | RPS20 |
| HLA-B1 | 0.748 | 0.780585064 | 0.496 | 0.904 | 0.818 | TCell | HLA-B |
| IL32 | 0.743 | 2.409338216 | 0.486 | 0.496 | 0.017 | TCell | IL32 |
| CCL5 | 0.737 | 2.986045807 | 0.474 | 0.496 | 0.037 | TCell | CCL5 |
| RPL322 | 0.736 | 0.684977138 | 0.472 | 0.917 | 0.866 | TCell | RPL32 |
| RPL23A | 0.735 | 1.140543624 | 0.47 | 0.732 | 0.57 | TCell | RPL23A |
| CD2 | 0.734 | 2.315889047 | 0.468 | 0.478 | 0.014 | TCell | CD2 |
| RPS32 | 0.732 | 0.735176247 | 0.464 | 0.874 | 0.806 | TCell | RPS3 |
| RPL371 | 0.72 | 0.747564825 | 0.44 | 0.847 | 0.791 | TCell | RPL37 |
| CD3D | 0.71 | 2.168472707 | 0.42 | 0.434 | 0.022 | TCell | CD3D |
| IL7R | 0.706 | 2.337417426 | 0.412 | 0.424 | 0.016 | TCell | IL7R |
| HLA-E1 | 0.705 | 0.806457829 | 0.41 | 0.75 | 0.603 | TCell | HLA-E |
| PFN1 | 0.695 | 1.171520041 | 0.39 | 0.618 | 0.42 | TCell | PFN1 |
| ARHGDIB | 0.693 | 1.670834663 | 0.386 | 0.475 | 0.141 | TCell | ARHGDIB |
| RPL39 | 0.69 | 0.801080666 | 0.38 | 0.749 | 0.669 | TCell | RPL39 |
| TSC22D31 | 0.688 | 1.054865456 | 0.376 | 0.624 | 0.398 | TCell | TSC22D3 |
| PTPRCAP | 0.685 | 1.821923375 | 0.37 | 0.404 | 0.048 | TCell | PTPRCAP |
| CD69 | 0.685 | 1.438066972 | 0.37 | 0.429 | 0.07 | TCell | CD69 |
| RPL14 | 0.683 | 0.71575244 | 0.366 | 0.754 | 0.702 | TCell | RPL14 |
| RGS11 | 0.681 | 1.147018703 | 0.362 | 0.52 | 0.186 | TCell | RGS1 |
| RPL102 | 0.679 | 0.798062143 | 0.358 | 0.733 | 0.626 | TCell | RPL10 |
| HLA-C | 0.679 | 0.716813358 | 0.358 | 0.745 | 0.664 | TCell | HLA-C |
| HLA-A | 0.677 | 0.817085104 | 0.354 | 0.69 | 0.581 | TCell | HLA-A |
| S100A4 | 0.676 | 0.998108679 | 0.352 | 0.595 | 0.348 | TCell | S100A4 |
| TNFAIP3 | 0.674 | 1.439349873 | 0.348 | 0.457 | 0.15 | TCell | TNFAIP3 |
| LCP1 | 0.67 | 1.640692964 | 0.34 | 0.381 | 0.052 | TCell | LCP1 |
| UBA52 | 0.67 | 0.74733757 | 0.34 | 0.695 | 0.628 | TCell | UBA52 |
| ETS1 | 0.664 | 1.638068248 | 0.328 | 0.37 | 0.06 | TCell | ETS1 |
| KLRB1 | 0.655 | 2.247715336 | 0.31 | 0.32 | 0.014 | TCell | KLRB1 |
| GZMA | 0.654 | 2.09997687 | 0.308 | 0.317 | 0.013 | TCell | GZMA |
| HLA-DRA1 | 0.98 | 3.49585012 | 0.96 | 0.974 | 0.227 | Myeloid | HLA-DRA |
| CD742 | 0.974 | 2.792351848 | 0.948 | 0.978 | 0.428 | Myeloid | CD74 |
| HLA-DRB11 | 0.958 | 3.151105833 | 0.916 | 0.94 | 0.17 | Myeloid | HLA-DRB1 |
| SRGN2 | 0.94 | 2.241361035 | 0.88 | 0.963 | 0.287 | Myeloid | SRGN |
| HLA-DPB1 | 0.922 | 3.181801351 | 0.844 | 0.862 | 0.079 | Myeloid | HLA-DPB1 |
| HLA-DPA11 | 0.921 | 3.198014515 | 0.842 | 0.867 | 0.11 | Myeloid | HLA-DPA1 |
| TMSB4X5 | 0.895 | 1.379972261 | 0.79 | 0.98 | 0.678 | Myeloid | TMSB4X |
| FTH11 | 0.894 | 1.510640292 | 0.788 | 0.964 | 0.716 | Myeloid | FTH1 |
| AIF1 | 0.871 | 2.729554447 | 0.742 | 0.746 | 0.009 | Myeloid | AIF1 |
| TMSB103 | 0.868 | 1.175323195 | 0.736 | 0.969 | 0.811 | Myeloid | TMSB10 |
| TYROBP | 0.866 | 2.521156104 | 0.732 | 0.748 | 0.025 | Myeloid | TYROBP |
| FTL2 | 0.857 | 1.639293626 | 0.714 | 0.972 | 0.821 | Myeloid | FTL |
| CST33 | 0.855 | 1.880578059 | 0.71 | 0.846 | 0.408 | Myeloid | CST3 |
| GPR183 | 0.854 | 2.59239267 | 0.708 | 0.735 | 0.048 | Myeloid | GPR183 |
| HLA-DQA1 | 0.849 | 2.878446015 | 0.698 | 0.715 | 0.04 | Myeloid | HLA-DQA1 |
| IFI30 | 0.844 | 2.569051351 | 0.688 | 0.719 | 0.072 | Myeloid | IFI30 |
| HLA-DRB51 | 0.843 | 2.497908574 | 0.686 | 0.723 | 0.08 | Myeloid | HLA-DRB5 |
| FGL2 | 0.821 | 2.395933152 | 0.642 | 0.654 | 0.02 | Myeloid | FGL2 |
| ACTB | 0.812 | 1.332552215 | 0.624 | 0.862 | 0.551 | Myeloid | ACTB |
| CTSS1 | 0.808 | 2.023386485 | 0.616 | 0.716 | 0.216 | Myeloid | CTSS |
| IL8 | 0.802 | 2.825846373 | 0.604 | 0.673 | 0.122 | Myeloid | IL8 |
| PLAUR | 0.8 | 2.356856388 | 0.6 | 0.634 | 0.06 | Myeloid | PLAUR |
| LAPTM5 | 0.8 | 1.872847677 | 0.6 | 0.646 | 0.054 | Myeloid | LAPTM5 |
| HLA-DQB1 | 0.798 | 2.252241525 | 0.596 | 0.615 | 0.033 | Myeloid | HLA-DQB1 |
| PSAP1 | 0.797 | 1.467049286 | 0.594 | 0.814 | 0.515 | Myeloid | PSAP |
| MS4A6A | 0.795 | 2.418292485 | 0.59 | 0.594 | 0.007 | Myeloid | MS4A6A |
| FCER1G | 0.79 | 2.007182771 | 0.58 | 0.596 | 0.019 | Myeloid | FCER1G |
| NFKBIA | 0.783 | 1.323708534 | 0.566 | 0.815 | 0.496 | Myeloid | NFKBIA |
| COTL1 | 0.78 | 1.984327668 | 0.56 | 0.598 | 0.05 | Myeloid | COTL1 |
| DUSP2 | 0.776 | 1.810286542 | 0.552 | 0.661 | 0.18 | Myeloid | DUSP2 |
| HLA-DMA | 0.771 | 1.891565815 | 0.542 | 0.594 | 0.088 | Myeloid | HLA-DMA |
| IL1B | 0.769 | 2.894686054 | 0.538 | 0.547 | 0.016 | Myeloid | IL1B |
| CPVL | 0.765 | 2.14314496 | 0.53 | 0.54 | 0.015 | Myeloid | CPVL |
| MNDA | 0.764 | 2.290776427 | 0.528 | 0.533 | 0.005 | Myeloid | MNDA |
| NAMPT | 0.759 | 1.606645772 | 0.518 | 0.636 | 0.185 | Myeloid | NAMPT |
| VIM4 | 0.754 | 0.933600797 | 0.508 | 0.783 | 0.333 | Myeloid | VIM |
| RGS2 | 0.751 | 1.631530782 | 0.502 | 0.614 | 0.152 | Myeloid | RGS2 |
| CD83 | 0.75 | 1.918948604 | 0.5 | 0.519 | 0.025 | Myeloid | CD83 |
| PTPRC1 | 0.745 | 1.437349182 | 0.49 | 0.561 | 0.075 | Myeloid | PTPRC |
| ITGB2 | 0.739 | 1.71811171 | 0.478 | 0.499 | 0.022 | Myeloid | ITGB2 |
| SH3BGRL3 | 0.738 | 1.369062932 | 0.476 | 0.628 | 0.239 | Myeloid | SH3BGRL3 |
| PLEK | 0.736 | 1.923671617 | 0.472 | 0.491 | 0.023 | Myeloid | PLEK |
| LST1 | 0.73 | 1.886495143 | 0.46 | 0.469 | 0.009 | Myeloid | LST1 |
| TNFAIP31 | 0.73 | 1.493925282 | 0.46 | 0.572 | 0.145 | Myeloid | TNFAIP3 |
| OAZ1 | 0.728 | 0.912360318 | 0.456 | 0.757 | 0.513 | Myeloid | OAZ1 |
| BCL2A1 | 0.724 | 2.402185933 | 0.448 | 0.472 | 0.032 | Myeloid | BCL2A1 |
| HLA-DMB | 0.722 | 1.72429834 | 0.444 | 0.461 | 0.024 | Myeloid | HLA-DMB |
| CLEC10A | 0.72 | 1.977368823 | 0.44 | 0.443 | 0.004 | Myeloid | CLEC10A |
| LCP11 | 0.717 | 1.525103954 | 0.434 | 0.48 | 0.048 | Myeloid | LCP1 |
| GPX1 | 0.717 | 1.135759744 | 0.434 | 0.635 | 0.314 | Myeloid | GPX1 |
| F13A1 | 0.713 | 2.307896165 | 0.426 | 0.433 | 0.008 | Myeloid | F13A1 |
| NPC2 | 0.713 | 1.188995753 | 0.426 | 0.63 | 0.321 | Myeloid | NPC2 |
| TPM31 | 0.706 | 1.078178168 | 0.412 | 0.613 | 0.281 | Myeloid | TPM3 |
| AMICA1 | 0.705 | 1.718058949 | 0.41 | 0.423 | 0.014 | Myeloid | AMICA1 |
| PFN11 | 0.705 | 0.93641031 | 0.41 | 0.683 | 0.418 | Myeloid | PFN1 |
| HLA-B2 | 0.705 | 0.529048116 | 0.41 | 0.915 | 0.818 | Myeloid | HLA-B |
| CCL3 | 0.704 | 2.35543246 | 0.408 | 0.436 | 0.035 | Myeloid | CCL3 |
| SAMSN1 | 0.702 | 1.709214024 | 0.404 | 0.457 | 0.058 | Myeloid | SAMSN1 |
| ZNF331 | 0.701 | 1.548703799 | 0.402 | 0.472 | 0.089 | Myeloid | ZNF331 |
| ARPC1B | 0.701 | 1.231251927 | 0.402 | 0.53 | 0.174 | Myeloid | ARPC1B |
| CYBB | 0.699 | 1.805016306 | 0.398 | 0.403 | 0.007 | Myeloid | CYBB |
| NR4A2 | 0.695 | 1.232714255 | 0.39 | 0.529 | 0.18 | Myeloid | NR4A2 |
| PPP1R15A1 | 0.693 | 0.833127864 | 0.386 | 0.757 | 0.555 | Myeloid | PPP1R15A |
| ARPC2 | 0.692 | 0.854167721 | 0.384 | 0.676 | 0.415 | Myeloid | ARPC2 |
| RGS12 | 0.691 | 1.196510866 | 0.382 | 0.546 | 0.186 | Myeloid | RGS1 |
| ARPC5 | 0.691 | 1.100147173 | 0.382 | 0.572 | 0.263 | Myeloid | ARPC5 |
| ARHGDIB1 | 0.688 | 1.068819831 | 0.376 | 0.496 | 0.14 | Myeloid | ARHGDIB |
| HLA-E2 | 0.688 | 0.596401704 | 0.376 | 0.805 | 0.6 | Myeloid | HLA-E |
| CTSH | 0.684 | 1.168617367 | 0.368 | 0.522 | 0.213 | Myeloid | CTSH |
| CD68 | 0.678 | 1.572425332 | 0.356 | 0.365 | 0.012 | Myeloid | CD68 |
| CTSB3 | 0.677 | 1.132751822 | 0.354 | 0.686 | 0.477 | Myeloid | CTSB |
| CD14 | 0.676 | 1.765079493 | 0.352 | 0.377 | 0.035 | Myeloid | CD14 |
| ACTR2 | 0.675 | 1.02301757 | 0.35 | 0.533 | 0.25 | Myeloid | ACTR2 |
| IGSF6 | 0.673 | 1.599810674 | 0.346 | 0.348 | 0.002 | Myeloid | IGSF6 |
| ARPC3 | 0.671 | 0.847110202 | 0.342 | 0.578 | 0.311 | Myeloid | ARPC3 |
| PTPRE | 0.669 | 1.45992938 | 0.338 | 0.366 | 0.033 | Myeloid | PTPRE |
| CFL1 | 0.669 | 0.746770376 | 0.338 | 0.634 | 0.413 | Myeloid | CFL1 |
| ATP5E | 0.669 | 0.599985867 | 0.338 | 0.739 | 0.575 | Myeloid | ATP5E |
| CD521 | 0.666 | 1.236493379 | 0.332 | 0.43 | 0.108 | Myeloid | CD52 |
| CLEC7A | 0.664 | 1.43804774 | 0.328 | 0.337 | 0.011 | Myeloid | CLEC7A |
| GRB2 | 0.659 | 0.967712777 | 0.318 | 0.49 | 0.218 | Myeloid | GRB2 |
| MS4A7 | 0.658 | 1.591317459 | 0.316 | 0.319 | 0.003 | Myeloid | MS4A7 |
| SAMHD11 | 0.655 | 1.220867684 | 0.31 | 0.391 | 0.099 | Myeloid | SAMHD1 |
| C5AR1 | 0.654 | 1.54863467 | 0.308 | 0.321 | 0.016 | Myeloid | C5AR1 |
| S100A41 | 0.654 | 0.711669192 | 0.308 | 0.603 | 0.348 | Myeloid | S100A4 |
| CXCL2 | 0.653 | 1.49036591 | 0.306 | 0.443 | 0.179 | Myeloid | CXCL2 |
| CTSC | 0.653 | 1.158253189 | 0.306 | 0.52 | 0.282 | Myeloid | CTSC |
| AREG | 0.652 | 1.471076028 | 0.304 | 0.356 | 0.062 | Myeloid | AREG |
| SOD2 | 0.652 | 1.202800691 | 0.304 | 0.493 | 0.256 | Myeloid | SOD2 |
| S100A9 | 0.651 | 2.121487638 | 0.302 | 0.379 | 0.098 | Myeloid | S100A9 |
| FCGRT | 0.651 | 1.019542231 | 0.302 | 0.435 | 0.168 | Myeloid | FCGRT |
| PABPC13 | 0.651 | 0.427083022 | 0.302 | 0.866 | 0.777 | Myeloid | PABPC1 |
| TPSAB1 | 0.998 | 5.172430408 | 0.996 | 0.996 | 0.026 | MastCell | TPSAB1 |
| CPA3 | 0.986 | 4.527062778 | 0.972 | 0.974 | 0.016 | MastCell | CPA3 |
| CD691 | 0.954 | 3.474079405 | 0.908 | 0.927 | 0.074 | MastCell | CD69 |
| SRGN3 | 0.944 | 2.248707999 | 0.888 | 0.974 | 0.307 | MastCell | SRGN |
| HPGD | 0.901 | 3.414958521 | 0.802 | 0.846 | 0.161 | MastCell | HPGD |
| RGS14 | 0.86 | 2.017268292 | 0.72 | 0.835 | 0.192 | MastCell | RGS1 |
| HPGDS | 0.843 | 2.942801994 | 0.686 | 0.689 | 0.007 | MastCell | HPGDS |
| SLC18A2 | 0.833 | 2.783946748 | 0.666 | 0.674 | 0.014 | MastCell | SLC18A2 |
| SAMSN11 | 0.822 | 2.262142376 | 0.644 | 0.685 | 0.066 | MastCell | SAMSN1 |
| KIT | 0.809 | 2.657829195 | 0.618 | 0.63 | 0.024 | MastCell | KIT |
| NFKBIZ | 0.806 | 1.781375389 | 0.612 | 0.74 | 0.274 | MastCell | NFKBIZ |
| HDC | 0.801 | 2.725190871 | 0.602 | 0.604 | 0.006 | MastCell | HDC |
| CTSG | 0.795 | 3.809693081 | 0.59 | 0.593 | 0.007 | MastCell | CTSG |
| ACSL4 | 0.793 | 2.244251452 | 0.586 | 0.641 | 0.113 | MastCell | ACSL4 |
| FTH12 | 0.789 | 0.995776186 | 0.578 | 0.908 | 0.725 | MastCell | FTH1 |
| LAPTM51 | 0.785 | 2.034293038 | 0.57 | 0.623 | 0.072 | MastCell | LAPTM5 |
| TMSB4X6 | 0.78 | 0.799937085 | 0.56 | 0.941 | 0.688 | MastCell | TMSB4X |
| TNFAIP32 | 0.777 | 1.863369041 | 0.554 | 0.652 | 0.157 | MastCell | TNFAIP3 |
| TPSD1 | 0.774 | 2.775624126 | 0.548 | 0.549 | 0.002 | MastCell | TPSD1 |
| CD522 | 0.768 | 1.756801512 | 0.536 | 0.626 | 0.115 | MastCell | CD52 |
| PTGS2 | 0.767 | 2.685715701 | 0.534 | 0.571 | 0.071 | MastCell | PTGS2 |
| GATA2 | 0.766 | 2.241628932 | 0.532 | 0.557 | 0.036 | MastCell | GATA2 |
| NFKBIA1 | 0.766 | 1.168531071 | 0.532 | 0.81 | 0.506 | MastCell | NFKBIA |
| PPP1R15A2 | 0.766 | 1.101140092 | 0.532 | 0.832 | 0.56 | MastCell | PPP1R15A |
| IL1RL1 | 0.76 | 2.23735727 | 0.52 | 0.524 | 0.006 | MastCell | IL1RL1 |
| VIM5 | 0.754 | 1.076149483 | 0.508 | 0.773 | 0.347 | MastCell | VIM |
| FTL3 | 0.745 | 0.572584353 | 0.49 | 0.938 | 0.826 | MastCell | FTL |
| DUSP6 | 0.741 | 1.850325933 | 0.482 | 0.593 | 0.199 | MastCell | DUSP6 |
| AHR | 0.739 | 1.594642188 | 0.478 | 0.601 | 0.205 | MastCell | AHR |
| MS4A2 | 0.734 | 2.180540203 | 0.468 | 0.469 | 0.002 | MastCell | MS4A2 |
| CD631 | 0.731 | 0.916593063 | 0.462 | 0.773 | 0.587 | MastCell | CD63 |
| NR4A21 | 0.727 | 1.618262465 | 0.454 | 0.575 | 0.19 | MastCell | NR4A2 |
| C1orf186 | 0.723 | 2.288276795 | 0.446 | 0.451 | 0.008 | MastCell | C1orf186 |
| VWA5A | 0.722 | 1.979038789 | 0.444 | 0.473 | 0.045 | MastCell | VWA5A |
| CLU5 | 0.72 | 1.126834693 | 0.44 | 0.659 | 0.326 | MastCell | CLU |
| AREG1 | 0.716 | 2.034196719 | 0.432 | 0.48 | 0.069 | MastCell | AREG |
| SELK1 | 0.716 | 1.26675522 | 0.432 | 0.645 | 0.352 | MastCell | SELK |
| RGS21 | 0.714 | 1.578995119 | 0.428 | 0.549 | 0.167 | MastCell | RGS2 |
| CCL4 | 0.712 | 2.362732077 | 0.424 | 0.476 | 0.073 | MastCell | CCL4 |
| ANXA15 | 0.703 | 0.674142772 | 0.406 | 0.864 | 0.691 | MastCell | ANXA1 |
| ALOX5AP | 0.696 | 1.782513984 | 0.392 | 0.418 | 0.031 | MastCell | ALOX5AP |
| GPR65 | 0.694 | 2.014983877 | 0.388 | 0.41 | 0.027 | MastCell | GPR65 |
| TYROBP1 | 0.694 | 1.412279245 | 0.388 | 0.44 | 0.052 | MastCell | TYROBP |
| GLUL2 | 0.694 | 1.0410677 | 0.388 | 0.681 | 0.462 | MastCell | GLUL |
| RGS13 | 0.692 | 2.159508305 | 0.384 | 0.388 | 0.004 | MastCell | RGS13 |
| S100A42 | 0.691 | 0.977544588 | 0.382 | 0.645 | 0.355 | MastCell | S100A4 |
| FOSB2 | 0.689 | 0.777847757 | 0.378 | 0.766 | 0.63 | MastCell | FOSB |
| CAPG | 0.682 | 1.573193904 | 0.364 | 0.425 | 0.087 | MastCell | CAPG |
| UBB1 | 0.682 | 0.695885498 | 0.364 | 0.821 | 0.751 | MastCell | UBB |
| TSC22D32 | 0.674 | 0.967831685 | 0.348 | 0.63 | 0.405 | MastCell | TSC22D3 |
| FCER1G1 | 0.669 | 1.477120755 | 0.338 | 0.374 | 0.04 | MastCell | FCER1G |
| PTMA2 | 0.669 | 0.626466055 | 0.338 | 0.784 | 0.698 | MastCell | PTMA |
| GCSAML | 0.668 | 1.739155075 | 0.336 | 0.337 | 0.001 | MastCell | GCSAML |
| ALAS1 | 0.667 | 1.848169473 | 0.334 | 0.396 | 0.091 | MastCell | ALAS1 |
| CTSD | 0.664 | 1.111244867 | 0.328 | 0.56 | 0.35 | MastCell | CTSD |
| NR4A11 | 0.659 | 0.772455625 | 0.318 | 0.648 | 0.482 | MastCell | NR4A1 |
| KLF61 | 0.658 | 0.802887837 | 0.316 | 0.703 | 0.546 | MastCell | KLF6 |
| RAC2 | 0.656 | 1.624379019 | 0.312 | 0.341 | 0.035 | MastCell | RAC2 |
| BTG2 | 0.654 | 0.720872821 | 0.308 | 0.689 | 0.529 | MastCell | BTG2 |
| ARHGDIB2 | 0.652 | 1.158381055 | 0.304 | 0.425 | 0.152 | MastCell | ARHGDIB |
| RP11-354E11.2 | 0.651 | 1.695420225 | 0.302 | 0.304 | 0.002 | MastCell | RP11-354E11.2 |

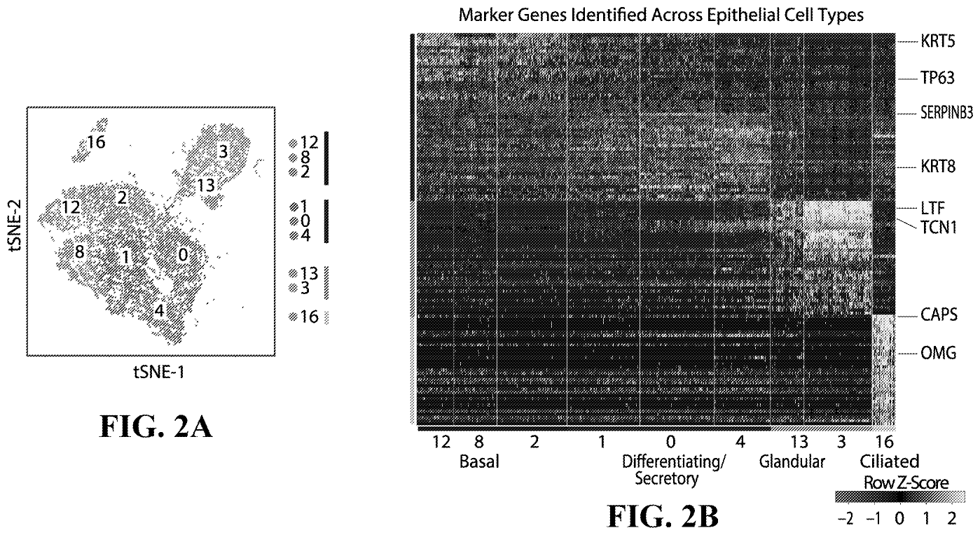

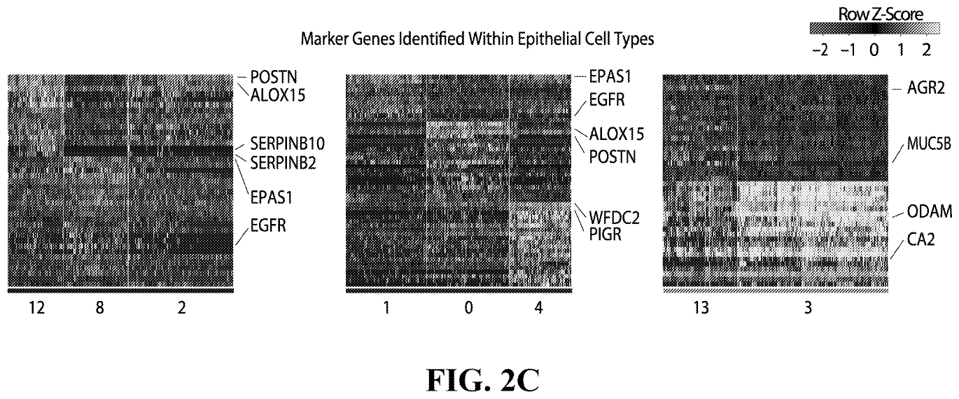

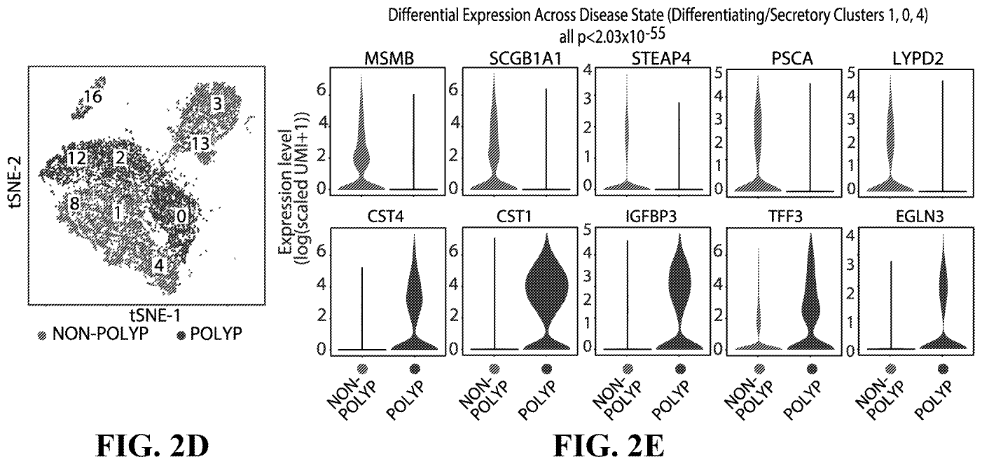

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 11. The clusters are as shown in FIG. 2B.

TABLE 12

| | mvAUC | avg_diff | power | pct. 1 | pct. 2 | cluster | gene |
|---|---|---|---|---|---|---|---|
| SERPINB3 | 0.837 | 1.6538909 | 0.674 | 0.895 | 0.474 | Apical | SERPINB3 |
| S100A6 | 0.786 | 0.788055508 | 0.572 | 0.974 | 0.832 | Apical | S100A6 |
| KRT19 | 0.754 | 0.694880412 | 0.508 | 0.955 | 0.758 | Apical | KRT19 |
| ANXA1 | 0.723 | 0.684481932 | 0.446 | 0.934 | 0.755 | Apical | ANXA1 |
| CLDN4 | 0.72 | 0.93803735 | 0.44 | 0.784 | 0.525 | Apical | CLDN4 |
| VMO1 | 0.716 | 1.360437483 | 0.432 | 0.63 | 0.279 | Apical | VMO1 |
| AGR2 | 0.714 | 0.732763858 | 0.428 | 0.814 | 0.546 | Apical | AGR2 |
| TSPAN1 | 0.697 | 0.800385782 | 0.394 | 0.613 | 0.244 | Apical | TSPAN1 |
| HSPB1 | 0.694 | 0.744867967 | 0.388 | 0.767 | 0.53 | Apical | HSPB1 |
| ELF3 | 0.691 | 0.688987512 | 0.382 | 0.763 | 0.516 | Apical | ELF3 |
| SERPINB4 | 0.686 | 0.987569307 | 0.372 | 0.589 | 0.24 | Apical | SERPINB4 |
| EPAS1 | 0.686 | 0.834740283 | 0.372 | 0.658 | 0.344 | Apical | EPAS1 |
| LGALS3 | 0.684 | 0.659864816 | 0.368 | 0.825 | 0.664 | Apical | LGALS3 |
| PRSS23 | 0.684 | 0.588212915 | 0.368 | 0.805 | 0.57 | Apical | PRSS23 |
| S100A4 | 0.68 | 1.343932433 | 0.36 | 0.512 | 0.201 | Apical | S100A4 |
| WFDC2 | 0.679 | 0.754490065 | 0.358 | 0.784 | 0.578 | Apical | WFDC2 |
| GSTP1 | 0.679 | 0.513550322 | 0.358 | 0.856 | 0.663 | Apical | GSTP1 |
| KRT8 | 0.678 | 0.701082845 | 0.356 | 0.727 | 0.506 | Apical | KRT8 |
| SAT1 | 0.678 | 0.568064854 | 0.356 | 0.875 | 0.738 | Apical | SAT1 |
| S100P | 0.668 | 1.371315688 | 0.336 | 0.468 | 0.165 | Apical | S100P |
| UGT2A2 | 0.668 | 0.799101685 | 0.336 | 0.58 | 0.27 | Apical | UGT2A2 |
| TXN | 0.667 | 0.660403813 | 0.334 | 0.728 | 0.539 | Apical | TXN |
| ANXA2 | 0.666 | 0.497348846 | 0.332 | 0.831 | 0.657 | Apical | ANXA2 |
| CST1 | 0.662 | 2.464147733 | 0.324 | 0.39 | 0.086 | Apical | CST1 |
| GABRP | 0.661 | 0.910625949 | 0.322 | 0.455 | 0.148 | Apical | GABRP |
| TMSB10 | 0.661 | 0.480813574 | 0.322 | 0.874 | 0.777 | Apical | TMSB10 |
| MGST1 | 0.656 | 0.743558953 | 0.312 | 0.53 | 0.254 | Apical | MGST1 |
| KRT18 | 0.652 | 0.584556313 | 0.304 | 0.703 | 0.516 | Apical | KRT18 |
| POSTN | 0.833 | 2.137335228 | 0.666 | 0.798 | 0.241 | Basal | POSTN |
| S100A2 | 0.827 | 1.367490728 | 0.654 | 0.877 | 0.363 | Basal | S100A2 |
| KRT5 | 0.806 | 1.389869235 | 0.612 | 0.775 | 0.241 | Basal | KRT5 |
| KRT15 | 0.803 | 1.881939141 | 0.606 | 0.698 | 0.154 | Basal | KRT15 |
| JUNB | 0.73 | 0.712412677 | 0.46 | 0.888 | 0.753 | Basal | JUNB |
| MMP10 | 0.727 | 1.650307947 | 0.454 | 0.584 | 0.176 | Basal | MMP10 |
| EGR1 | 0.713 | 0.672249017 | 0.426 | 0.867 | 0.69 | Basal | EGR1 |
| MIR205HG | 0.71 | 1.141784267 | 0.42 | 0.568 | 0.181 | Basal | MIR205HG |
| KRT17 | 0.707 | 1.249732868 | 0.414 | 0.526 | 0.134 | Basal | KRT17 |
| TNC | 0.687 | 1.104107244 | 0.374 | 0.506 | 0.15 | Basal | TNC |
| RPL3 | 0.687 | 0.521974505 | 0.374 | 0.896 | 0.803 | Basal | RPL3 |
| ETS2 | 0.686 | 0.857033246 | 0.372 | 0.666 | 0.393 | Basal | ETS2 |
| DST | 0.682 | 1.085538422 | 0.364 | 0.529 | 0.204 | Basal | DST |
| SERPINF1 | 0.674 | 0.997237999 | 0.348 | 0.502 | 0.184 | Basal | SERPINF1 |
| TP63 | 0.671 | 1.144942276 | 0.342 | 0.412 | 0.075 | Basal | TP63 |
| RPL13A | 0.671 | 0.437361856 | 0.342 | 0.931 | 0.856 | Basal | RPL13A |
| RPS25 | 0.667 | 0.522751781 | 0.334 | 0.841 | 0.755 | Basal | RPS25 |
| EIF1 | 0.667 | 0.447611948 | 0.334 | 0.882 | 0.812 | Basal | EIF1 |
| IFITM3 | 0.659 | 0.939305541 | 0.318 | 0.519 | 0.262 | Basal | IFITM3 |
| IL33 | 0.657 | 0.93405925 | 0.314 | 0.502 | 0.225 | Basal | IL33 |
| LAMB3 | 0.656 | 1.089073959 | 0.312 | 0.402 | 0.104 | Basal | LAMB3 |
| RPL10A | 0.656 | 0.465560908 | 0.312 | 0.818 | 0.721 | Basal | RPL10A |
| RPL4 | 0.652 | 0.451832501 | 0.304 | 0.808 | 0.737 | Basal | RPL4 |
| BTF3 | 0.651 | 0.539069481 | 0.302 | 0.744 | 0.636 | Basal | BTF3 |
| RPS9 | 0.651 | 0.412024933 | 0.302 | 0.854 | 0.782 | Basal | RPS9 |
| CAPS | 0.938 | 2.780690693 | 0.876 | 0.91 | 0.209 | Ciliated | CAPS |
| C9orf24 | 0.909 | 2.873906899 | 0.818 | 0.823 | 0.017 | Ciliated | C9orf24 |
| PIFO | 0.904 | 2.502745184 | 0.808 | 0.813 | 0.015 | Ciliated | PIFO |
| TPPP3 | 0.898 | 2.608289689 | 0.796 | 0.801 | 0.014 | Ciliated | TPPP3 |
| C20orf85 | 0.895 | 2.567424034 | 0.79 | 0.793 | 0.007 | Ciliated | C20orf85 |
| TUBA1A | 0.882 | 2.343583629 | 0.764 | 0.805 | 0.099 | Ciliated | TUBA1A |
| TSPAN12 | 0.877 | 1.613918786 | 0.754 | 0.908 | 0.375 | Ciliated | TSPAN1 |
| SNTN | 0.873 | 2.67890619 | 0.746 | 0.749 | 0.007 | Ciliated | SNTN |
| FAM183A | 0.864 | 2.291478715 | 0.728 | 0.733 | 0.009 | Ciliated | FAM183A |
| TUBB4B | 0.85 | 1.683292107 | 0.7 | 0.819 | 0.268 | Ciliated | TUBB4B |
| C11orf88 | 0.84 | 2.20836905 | 0.68 | 0.687 | 0.01 | Ciliated | C11orf88 |
| RSPH1 | 0.84 | 2.140983747 | 0.68 | 0.685 | 0.007 | Ciliated | RSPH1 |
| OMG | 0.839 | 2.533187223 | 0.678 | 0.683 | 0.01 | Ciliated | OMG |
| GSTA1 | 0.839 | 2.508811872 | 0.678 | 0.711 | 0.07 | Ciliated | GSTA1 |
| CAPSL | 0.838 | 2.15037214 | 0.676 | 0.679 | 0.004 | Ciliated | CAPSL |
| CCDC170 | 0.836 | 1.993744502 | 0.672 | 0.677 | 0.007 | Ciliated | CCDC170 |
| AGR3 | 0.834 | 2.189825958 | 0.668 | 0.693 | 0.041 | Ciliated | AGR3 |
| IFT57 | 0.831 | 1.84764937 | 0.662 | 0.715 | 0.094 | Ciliated | IFT57 |
| DYNLL1 | 0.827 | 1.351963816 | 0.654 | 0.839 | 0.343 | Ciliated | DYNLL1 |
| DNAH5 | 0.825 | 1.968845916 | 0.65 | 0.661 | 0.017 | Ciliated | DNAH5 |
| PRDX5 | 0.825 | 1.384136595 | 0.65 | 0.841 | 0.47 | Ciliated | PRDX5 |
| CIB1 | 0.823 | 1.448911506 | 0.646 | 0.807 | 0.314 | Ciliated | CIB1 |
| TMEM190 | 0.82 | 2.24383314 | 0.64 | 0.643 | 0.005 | Ciliated | TMEM190 |
| DYNLT1 | 0.82 | 1.571604523 | 0.64 | 0.765 | 0.242 | Ciliated | DYNLT1 |
| HSP90AA1 | 0.819 | 1.14658247 | 0.638 | 0.908 | 0.616 | Ciliated | HSP90AA1 |
| C1orf194 | 0.818 | 1.944786988 | 0.636 | 0.641 | 0.006 | Ciliated | C1orf194 |
| PSENEN | 0.816 | 1.815499744 | 0.632 | 0.697 | 0.113 | Ciliated | PSENEN |
| RP11-356K23.1 | 0.814 | 2.234448469 | 0.628 | 0.633 | 0.008 | Ciliated | RP11-356K23.1 |
| MORN2 | 0.814 | 1.798896518 | 0.628 | 0.657 | 0.044 | Ciliated | MORN2 |
| SPA17 | 0.812 | 1.898333308 | 0.624 | 0.635 | 0.016 | Ciliated | SPA17 |
| CALM1 | 0.809 | 1.009615265 | 0.618 | 0.9 | 0.631 | Ciliated | CALM1 |
| C9orf116 | 0.808 | 1.940088998 | 0.616 | 0.622 | 0.011 | Ciliated | C9orf116 |
| ATPIF1 | 0.808 | 1.29341648 | 0.616 | 0.825 | 0.426 | Ciliated | ATPIF1 |
| NUCB21 | 0.807 | 1.265740491 | 0.614 | 0.809 | 0.295 | Ciliated | NUCB2 |
| ZMYND10 | 0.802 | 1.81504398 | 0.604 | 0.608 | 0.006 | Ciliated | ZMYND10 |
| ROPN1L | 0.801 | 1.847228389 | 0.602 | 0.606 | 0.005 | Ciliated | ROPN1L |
| CETN2 | 0.801 | 1.711504581 | 0.602 | 0.639 | 0.055 | Ciliated | CETN2 |
| DNAH12 | 0.799 | 1.868314331 | 0.598 | 0.6 | 0.004 | Ciliated | DNAH12 |
| LRRIQ1 | 0.798 | 1.896492505 | 0.596 | 0.6 | 0.006 | Ciliated | LRRIQ1 |
| C5orf49 | 0.796 | 1.803203709 | 0.592 | 0.594 | 0.004 | Ciliated | C5orf49 |
| C1orf173 | 0.784 | 1.736694238 | 0.568 | 0.57 | 0.003 | Ciliated | C1orf173 |
| CCDC146 | 0.781 | 1.654020934 | 0.562 | 0.594 | 0.042 | Ciliated | CCDC146 |
| TCTEX1D4 | 0.779 | 1.84432792 | 0.558 | 0.562 | 0.007 | Ciliated | TCTEX1D4 |
| TMC5 | 0.776 | 1.509865035 | 0.552 | 0.629 | 0.112 | Ciliated | TMC5 |
| PLAC8 | 0.775 | 1.321158725 | 0.55 | 0.675 | 0.17 | Ciliated | PLAC8 |
| FAM216B | 0.774 | 1.719172509 | 0.548 | 0.55 | 0.002 | Ciliated | FAM216B |
| SPAG6 | 0.773 | 1.681184681 | 0.546 | 0.548 | 0.003 | Ciliated | SPAG6 |
| CALM2 | 0.773 | 0.836779389 | 0.546 | 0.892 | 0.636 | Ciliated | CALM2 |
| FAM154B | 0.771 | 1.632138519 | 0.542 | 0.546 | 0.006 | Ciliated | FAM154B |
| FAM81B | 0.769 | 1.680748409 | 0.538 | 0.54 | 0.002 | Ciliated | FAM81B |
| FAM229B | 0.768 | 1.548021924 | 0.536 | 0.564 | 0.037 | Ciliated | FAM229B |
| CTSS | 0.768 | 1.320516168 | 0.536 | 0.661 | 0.187 | Ciliated | CTSS |
| EZR | 0.767 | 0.771689433 | 0.534 | 0.91 | 0.681 | Ciliated | EZR |
| EFCAB1 | 0.766 | 1.598806831 | 0.532 | 0.54 | 0.009 | Ciliated | EFCAB1 |
| CD59 | 0.766 | 1.04877214 | 0.532 | 0.793 | 0.43 | Ciliated | CD59 |
| ARL3 | 0.764 | 1.262375173 | 0.528 | 0.677 | 0.215 | Ciliated | ARL3 |
| ARMC3 | 0.76 | 1.582316902 | 0.52 | 0.522 | 0.003 | Ciliated | ARMC3 |
| IQCG | 0.758 | 1.487414939 | 0.516 | 0.532 | 0.02 | Ciliated | IQCG |
| IK | 0.758 | 1.259068105 | 0.516 | 0.637 | 0.161 | Ciliated | IK |
| CDHR3 | 0.756 | 1.696094388 | 0.512 | 0.516 | 0.006 | Ciliated | CDHR3 |
| FAM92B | 0.756 | 1.554533336 | 0.512 | 0.514 | 0.003 | Ciliated | FAM92B |
| FOXJ1 | 0.755 | 1.520288741 | 0.51 | 0.52 | 0.01 | Ciliated | FOXJ1 |
| RRAD | 0.754 | 1.57081728 | 0.508 | 0.526 | 0.023 | Ciliated | RRAD |
| NQO1 | 0.753 | 1.270936455 | 0.506 | 0.602 | 0.122 | Ciliated | NQO1 |
| TSPAN19 | 0.752 | 1.692177148 | 0.504 | 0.508 | 0.004 | Ciliated | TSPAN19 |
| DYDC2 | 0.75 | 1.625104135 | 0.5 | 0.502 | 0.003 | Ciliated | DYDC2 |
| RSPH4A | 0.75 | 1.524135549 | 0.5 | 0.502 | 0.003 | Ciliated | RSPH4A |
| ODF3B | 0.748 | 1.539755336 | 0.496 | 0.524 | 0.036 | Ciliated | ODF3B |
| DNALI1 | 0.747 | 1.496481182 | 0.494 | 0.502 | 0.01 | Ciliated | DNALI1 |
| UFC1 | 0.747 | 1.220492832 | 0.494 | 0.62 | 0.164 | Ciliated | UFC1 |
| TEKT1 | 0.746 | 1.424438625 | 0.492 | 0.494 | 0.002 | Ciliated | TEKT1 |
| SMIM22 | 0.746 | 1.234057754 | 0.492 | 0.61 | 0.16 | Ciliated | SMIM22 |
| CHST9 | 0.746 | 1.111227814 | 0.492 | 0.663 | 0.226 | Ciliated | CHST9 |
| NME5 | 0.745 | 1.440386805 | 0.49 | 0.504 | 0.017 | Ciliated | NME5 |
| CYP4B1 | 0.745 | 1.182481742 | 0.49 | 0.679 | 0.248 | Ciliated | CYP4B1 |
| PCM1 | 0.743 | 1.103671406 | 0.486 | 0.671 | 0.24 | Ciliated | PCM1 |
| C9orf135 | 0.742 | 1.46701535 | 0.484 | 0.49 | 0.007 | Ciliated | C9orf135 |
| WDR78 | 0.742 | 1.450928056 | 0.484 | 0.492 | 0.01 | Ciliated | WDR78 |
| ODF2L | 0.741 | 1.308977164 | 0.482 | 0.56 | 0.092 | Ciliated | ODF2L |
| SCGB2A1 | 0.74 | 1.443371792 | 0.48 | 0.56 | 0.095 | Ciliated | SCGB2A1 |
| ABCA13 | 0.736 | 1.133412229 | 0.472 | 0.618 | 0.172 | Ciliated | ABCA13 |
| ALDH3B1 | 0.735 | 1.371051807 | 0.47 | 0.5 | 0.037 | Ciliated | ALDH3B1 |
| LRRC23 | 0.734 | 1.338921628 | 0.468 | 0.482 | 0.015 | Ciliated | LRRC23 |
| CKB | 0.734 | 1.279311478 | 0.468 | 0.558 | 0.104 | Ciliated | CKB |
| HSPH1 | 0.733 | 1.164359446 | 0.466 | 0.6 | 0.157 | Ciliated | HSPH1 |
| SLC44A4 | 0.732 | 1.214430919 | 0.464 | 0.558 | 0.113 | Ciliated | SLC44A4 |
| MS4A8 | 0.731 | 1.458135313 | 0.462 | 0.468 | 0.006 | Ciliated | MS4A8 |
| WDR52 | 0.731 | 1.421268503 | 0.462 | 0.49 | 0.032 | Ciliated | WDR52 |
| ENKUR | 0.73 | 1.499011574 | 0.46 | 0.462 | 0.002 | Ciliated | ENKUR |
| EFHC1 | 0.728 | 1.381019957 | 0.456 | 0.472 | 0.018 | Ciliated | EFHC1 |
| ZBBX | 0.725 | 1.376643267 | 0.45 | 0.452 | 0.001 | Ciliated | ZBBX |
| NUDC | 0.725 | 1.102889541 | 0.45 | 0.554 | 0.127 | Ciliated | NUDC |
| RSPH9 | 0.724 | 1.513409386 | 0.448 | 0.452 | 0.005 | Ciliated | RSPH9 |
| C1orf192 | 0.724 | 1.368684565 | 0.448 | 0.45 | 0.002 | Ciliated | C1orf192 |
| IGFBP7 | 0.724 | 1.173381256 | 0.448 | 0.633 | 0.231 | Ciliated | IGFBP7 |
| GSTP11 | 0.723 | 0.585962998 | 0.446 | 0.936 | 0.735 | Ciliated | GSTP1 |
| CCDC11 | 0.722 | 1.388337282 | 0.444 | 0.45 | 0.006 | Ciliated | CCDC11 |
| CCDC113 | 0.721 | 1.338858758 | 0.442 | 0.454 | 0.013 | Ciliated | CCDC113 |
| C12orf75 | 0.721 | 1.210482134 | 0.442 | 0.49 | 0.05 | Ciliated | C12orf75 |
| WDR54 | 0.72 | 1.367025502 | 0.44 | 0.458 | 0.019 | Ciliated | WDR54 |
| AK7 | 0.72 | 1.341482615 | 0.44 | 0.448 | 0.008 | Ciliated | AK7 |
| EFCAB10 | 0.72 | 1.292429388 | 0.44 | 0.442 | 0.002 | Ciliated | EFCAB10 |
| LDLRAD1 | 0.719 | 1.418373481 | 0.438 | 0.444 | 0.006 | Ciliated | LDLRAD1 |
| WDR96 | 0.719 | 1.404005839 | 0.438 | 0.44 | 0.003 | Ciliated | WDR96 |
| KIF9 | 0.719 | 1.29551357 | 0.438 | 0.46 | 0.026 | Ciliated | KIF9 |
| MLF1 | 0.719 | 1.226110528 | 0.438 | 0.488 | 0.056 | Ciliated | MLF1 |
| SPAG16 | 0.719 | 1.18957689 | 0.438 | 0.534 | 0.116 | Ciliated | SPAG16 |
| SYNE1 | 0.717 | 1.407256862 | 0.434 | 0.448 | 0.014 | Ciliated | SYNE1 |
| DYNLRB2 | 0.717 | 1.316569117 | 0.434 | 0.44 | 0.006 | Ciliated | DYNLRB2 |
| WDR86-AS1 | 0.716 | 1.433668551 | 0.432 | 0.438 | 0.006 | Ciliated | WDR86-AS1 |
| TSPAN6 | 0.716 | 1.095781924 | 0.432 | 0.556 | 0.157 | Ciliated | TSPAN6 |
| AKAP14 | 0.714 | 1.379478549 | 0.428 | 0.43 | 0.002 | Ciliated | AKAP14 |
| C10orf107 | 0.713 | 1.372504422 | 0.426 | 0.428 | 0.002 | Ciliated | C10orf107 |
| C11orf70 | 0.713 | 1.321148075 | 0.426 | 0.436 | 0.012 | Ciliated | C11orf70 |
| MNS1 | 0.713 | 1.312629445 | 0.426 | 0.432 | 0.006 | Ciliated | MNS1 |
| SPEF2 | 0.713 | 1.290293829 | 0.426 | 0.442 | 0.019 | Ciliated | SPEF2 |
| CCDC17 | 0.712 | 1.391862019 | 0.424 | 0.428 | 0.004 | Ciliated | CCDC17 |
| NPHP1 | 0.711 | 1.296212149 | 0.422 | 0.436 | 0.016 | Ciliated | NPHP1 |
| PPIL6 | 0.71 | 1.285641261 | 0.42 | 0.426 | 0.007 | Ciliated | PPIL6 |
| CCDC19 | 0.709 | 1.281219585 | 0.418 | 0.42 | 0.002 | Ciliated | CCDC19 |
| TMEM231 | 0.709 | 1.225243522 | 0.418 | 0.428 | 0.011 | Ciliated | TMEM231 |
| SPATA18 | 0.709 | 1.219910943 | 0.418 | 0.44 | 0.024 | Ciliated | SPATA18 |
| GSTA2 | 0.708 | 1.892121461 | 0.416 | 0.422 | 0.007 | Ciliated | GSTA2 |
| C6orf118 | 0.708 | 1.306576351 | 0.416 | 0.418 | 0.002 | Ciliated | C6orf118 |
| STOML3 | 0.708 | 1.259363294 | 0.416 | 0.418 | 0.001 | Ciliated | STOML3 |
| C14orf142 | 0.708 | 1.197377247 | 0.416 | 0.456 | 0.045 | Ciliated | C14orf142 |
| FANK1 | 0.708 | 1.172027285 | 0.416 | 0.422 | 0.007 | Ciliated | FANK1 |
| HSPB11 | 0.708 | 1.040621416 | 0.416 | 0.554 | 0.16 | Ciliated | HSPB11 |
| TCTEX1D2 | 0.707 | 1.282976203 | 0.414 | 0.444 | 0.034 | Ciliated | TCTEX1D2 |
| ARHGAP18 | 0.707 | 1.110251209 | 0.414 | 0.524 | 0.13 | Ciliated | ARHGAP18 |
| C21orf59 | 0.707 | 1.102906435 | 0.414 | 0.494 | 0.093 | Ciliated | C21orf59 |
| HMGN3 | 0.705 | 0.910819808 | 0.41 | 0.655 | 0.303 | Ciliated | HMGN3 |
| SPAG1 | 0.704 | 1.222859973 | 0.408 | 0.434 | 0.028 | Ciliated | SPAG1 |
| CSPP1 | 0.704 | 1.210809157 | 0.408 | 0.454 | 0.049 | Ciliated | CSPP1 |
| MRPS31 | 0.704 | 1.177851538 | 0.408 | 0.498 | 0.108 | Ciliated | MRPS31 |
| RP11-867G2.2 | 0.701 | 1.291770093 | 0.402 | 0.404 | 0.001 | Ciliated | RP11-867G2.2 |
| CES1 | 0.7 | 1.1893514 | 0.4 | 0.466 | 0.075 | Ciliated | CES1 |
| NEK10 | 0.699 | 1.3340344 | 0.398 | 0.4 | 0.002 | Ciliated | NEK10 |
| CCDC39 | 0.699 | 1.280643789 | 0.398 | 0.402 | 0.004 | Ciliated | CCDC39 |
| ANKUB1 | 0.699 | 1.280255059 | 0.398 | 0.402 | 0.005 | Ciliated | ANKUB1 |
| PSCA | 0.699 | 0.886381619 | 0.398 | 0.55 | 0.152 | Ciliated | PSCA |
| DNAAF1 | 0.698 | 1.407026532 | 0.396 | 0.404 | 0.008 | Ciliated | DNAAF1 |
| CATSPERD | 0.698 | 1.336883714 | 0.396 | 0.398 | 0.002 | Ciliated | CATSPERD |
| SAMHD1 | 0.698 | 0.983878952 | 0.396 | 0.488 | 0.1 | Ciliated | SAMHD1 |
| DPY30 | 0.698 | 0.943815893 | 0.396 | 0.554 | 0.184 | Ciliated | DPY30 |
| MORN5 | 0.697 | 1.323224101 | 0.394 | 0.396 | 0.002 | Ciliated | MORN5 |
| DPCD | 0.695 | 1.069790768 | 0.39 | 0.428 | 0.041 | Ciliated | DPCD |
| AKAP9 | 0.695 | 0.771752814 | 0.39 | 0.709 | 0.378 | Ciliated | AKAP9 |
| B9D1 | 0.694 | 1.247087011 | 0.388 | 0.408 | 0.023 | Ciliated | B9D1 |
| TAX1BP1 | 0.694 | 0.748351235 | 0.388 | 0.691 | 0.374 | Ciliated | TAX1BP1 |
| DHRS9 | 0.693 | 1.082947114 | 0.386 | 0.462 | 0.085 | Ciliated | DHRS9 |
| PRDX1 | 0.693 | 0.579195361 | 0.386 | 0.847 | 0.654 | Ciliated | PRDX1 |
| SEPW1 | 0.692 | 0.819667678 | 0.384 | 0.647 | 0.335 | Ciliated | SEPW1 |
| KIF21A | 0.691 | 0.896657556 | 0.382 | 0.57 | 0.208 | Ciliated | KIF21A |
| HSBP1 | 0.691 | 0.713414752 | 0.382 | 0.677 | 0.366 | Ciliated | HSBP1 |
| SPAG17 | 0.69 | 1.280510767 | 0.38 | 0.382 | 0.002 | Ciliated | SPAG17 |
| DNPH1 | 0.69 | 0.956715673 | 0.38 | 0.51 | 0.15 | Ciliated | DNPH1 |
| MAP3K19 | 0.689 | 1.195392093 | 0.378 | 0.38 | 0.001 | Ciliated | MAP3K19 |
| LRRC48 | 0.689 | 1.1752053 | 0.378 | 0.384 | 0.006 | Ciliated | LRRC48 |
| WDR66 | 0.689 | 1.154667744 | 0.378 | 0.39 | 0.012 | Ciliated | WDR66 |
| LRRC46 | 0.689 | 1.153433531 | 0.378 | 0.382 | 0.003 | Ciliated | LRRC46 |
| RP11-275I14.4 | 0.688 | 1.212062804 | 0.376 | 0.384 | 0.008 | Ciliated | RP11-275I14.4 |
| EFCAB2 | 0.687 | 1.105950649 | 0.374 | 0.4 | 0.029 | Ciliated | EFCAB2 |
| NWD1 | 0.687 | 1.041962067 | 0.374 | 0.43 | 0.057 | Ciliated | NWD1 |
| IFI27 | 0.687 | 0.814981051 | 0.374 | 0.637 | 0.299 | Ciliated | IFI27 |
| C9orf117 | 0.686 | 1.318276884 | 0.372 | 0.373 | 0.003 | Ciliated | C9orf117 |
| LINC00948 | 0.686 | 1.289801152 | 0.372 | 0.376 | 0.003 | Ciliated | LINC00948 |
| CTGF | 0.686 | 0.98216839 | 0.372 | 0.478 | 0.107 | Ciliated | CTGF |
| UBB | 0.686 | 0.503364478 | 0.372 | 0.882 | 0.758 | Ciliated | UBB |
| LGALS31 | 0.686 | 0.493457682 | 0.372 | 0.908 | 0.723 | Ciliated | LGALS3 |
| DNAH3 | 0.684 | 1.165983345 | 0.368 | 0.369 | 0.002 | Ciliated | DNAH3 |
| PIH1D2 | 0.683 | 1.160029243 | 0.366 | 0.371 | 0.006 | Ciliated | PIH1D2 |
| FHAD1 | 0.683 | 1.146682857 | 0.366 | 0.378 | 0.012 | Ciliated | FHAD1 |
| TSTD1 | 0.683 | 0.79806697 | 0.366 | 0.612 | 0.301 | Ciliated | TSTD1 |
| DNAJA4 | 0.682 | 1.13925277 | 0.364 | 0.394 | 0.033 | Ciliated | DNAJA4 |
| OSCP1 | 0.682 | 1.095676831 | 0.364 | 0.378 | 0.015 | Ciliated | 0SCP1 |
| DTHD1 | 0.681 | 1.236575989 | 0.362 | 0.363 | 0.001 | Ciliated | DTHD1 |
| CCDC173 | 0.681 | 1.226344404 | 0.362 | 0.365 | 0.004 | Ciliated | CCDC173 |
| CYSTM1 | 0.681 | 0.816972302 | 0.362 | 0.536 | 0.195 | Ciliated | CYSTM1 |
| DNAH9 | 0.68 | 1.115482369 | 0.36 | 0.363 | 0.003 | Ciliated | DNAH9 |
| RABL5 | 0.68 | 1.030763358 | 0.36 | 0.396 | 0.04 | Ciliated | RABL5 |
| CCDC42B | 0.679 | 1.14585146 | 0.358 | 0.361 | 0.004 | Ciliated | CCDC42B |
| CCDC176 | 0.679 | 1.054772752 | 0.358 | 0.373 | 0.016 | Ciliated | CCDC176 |
| FAM174A | 0.679 | 1.02099957 | 0.358 | 0.43 | 0.078 | Ciliated | FAM174A |
| CCDC65 | 0.677 | 1.113831571 | 0.354 | 0.355 | 0.002 | Ciliated | CCDC65 |
| SRI | 0.677 | 0.78544428 | 0.354 | 0.596 | 0.288 | Ciliated | SRI |
| DRC1 | 0.676 | 1.148340043 | 0.352 | 0.353 | 0.001 | Ciliated | DRCI |
| CDS1 | 0.676 | 1.00784527 | 0.352 | 0.402 | 0.053 | Ciliated | CDS1 |
| LZTFL1 | 0.676 | 1.000309317 | 0.352 | 0.398 | 0.049 | Ciliated | LZTFL1 |
| C11orf74 | 0.675 | 1.062922991 | 0.35 | 0.384 | 0.034 | Ciliated | C11orf74 |
| CASC1 | 0.675 | 1.057025551 | 0.35 | 0.355 | 0.006 | Ciliated | CASC1 |
| CLU | 0.673 | 0.592176658 | 0.346 | 0.667 | 0.375 | Ciliated | CLU |
| IGFBP2 | 0.672 | 0.810864837 | 0.344 | 0.452 | 0.107 | Ciliated | IGFBP2 |
| CD164 | 0.672 | 0.726348451 | 0.344 | 0.643 | 0.362 | Ciliated | CD164 |
| PROM1 | 0.671 | 0.993184595 | 0.342 | 0.422 | 0.09 | Ciliated | PROM1 |
| TRAF3IP1 | 0.67 | 0.972537112 | 0.34 | 0.384 | 0.045 | Ciliated | TRAF3IP1 |
| CCDC104 | 0.669 | 0.925015284 | 0.338 | 0.422 | 0.093 | Ciliated | CCDC104 |
| ARMC4 | 0.668 | 1.082115293 | 0.336 | 0.337 | 0.001 | Ciliated | ARMC4 |
| DZIP3 | 0.667 | 0.996390519 | 0.334 | 0.365 | 0.032 | Ciliated | DZIP3 |
| STK33 | 0.666 | 1.101705331 | 0.332 | 0.343 | 0.012 | Ciliated | STK33 |
| UBXN10 | 0.666 | 1.024728884 | 0.332 | 0.335 | 0.003 | Ciliated | UBXN10 |
| TSPAN3 | 0.666 | 0.610830172 | 0.332 | 0.641 | 0.347 | Ciliated | TSPAN3 |
| C7orf57 | 0.664 | 1.018769824 | 0.328 | 0.329 | 0.002 | Ciliated | C7orf57 |
| IFT43 | 0.664 | 0.912687121 | 0.328 | 0.394 | 0.068 | Ciliated | IFT43 |
| DNAH11 | 0.663 | 1.145839115 | 0.326 | 0.333 | 0.007 | Ciliated | DNAH11 |
| TTC29 | 0.663 | 1.09548142 | 0.326 | 0.327 | 0.001 | Ciliated | TTC29 |
| TTC18 | 0.662 | 1.13085126 | 0.324 | 0.341 | 0.018 | Ciliated | TTC18 |
| PPP1R42 | 0.662 | 1.065949701 | 0.324 | 0.327 | 0.003 | Ciliated | PPP1R42 |
| TNFAIP8L1 | 0.661 | 1.046513317 | 0.322 | 0.329 | 0.007 | Ciliated | TNFAIP8L1 |
| TCTN1 | 0.66 | 0.897222514 | 0.32 | 0.351 | 0.032 | Ciliated | TCTN1 |
| IFT172 | 0.66 | 0.857117179 | 0.32 | 0.394 | 0.078 | Ciliated | IFT172 |
| CCDC78 | 0.659 | 0.991705251 | 0.318 | 0.321 | 0.002 | Ciliated | CCDC78 |
| CC2D2A | 0.659 | 0.988153998 | 0.318 | 0.329 | 0.012 | Ciliated | CC2D2A |
| LRP11 | 0.659 | 0.857786775 | 0.318 | 0.39 | 0.075 | Ciliated | LRP11 |
| CDHR4 | 0.658 | 1.052736096 | 0.316 | 0.317 | 0.002 | Ciliated | CDHR4 |
| IFT81 | 0.658 | 0.81300686 | 0.316 | 0.388 | 0.071 | Ciliated | IFT81 |
| ECT2L | 0.657 | 1.047255764 | 0.314 | 0.315 | 0.002 | Ciliated | ECT2L |
| DNAL1 | 0.657 | 0.959653049 | 0.314 | 0.337 | 0.026 | Ciliated | DNAL1 |
| RUVBL2 | 0.657 | 0.957273423 | 0.314 | 0.373 | 0.065 | Ciliated | RUVBL2 |
| RUVBL1 | 0.657 | 0.880725631 | 0.314 | 0.353 | 0.041 | Ciliated | RUVBL1 |
| H2AFJ | 0.656 | 0.67000694 | 0.312 | 0.544 | 0.255 | Ciliated | H2AFJ |
| DSTN | 0.656 | 0.527796212 | 0.312 | 0.711 | 0.515 | Ciliated | DSTN |
| ALDH1A1 | 0.656 | 0.495833698 | 0.312 | 0.779 | 0.551 | Ciliated | ALDH1A1 |
| IQCD | 0.655 | 0.927631458 | 0.31 | 0.313 | 0.004 | Ciliated | IQCD |
| POLR2I | 0.655 | 0.773997255 | 0.31 | 0.44 | 0.14 | Ciliated | POLR2I |
| PTGES3 | 0.655 | 0.645221636 | 0.31 | 0.53 | 0.243 | Ciliated | PTGES3 |
| DNAH7 | 0.654 | 1.108636805 | 0.308 | 0.315 | 0.008 | Ciliated | DNAH7 |
| RP4-666F24.3 | 0.654 | 1.014193908 | 0.308 | 0.309 | 0.002 | Ciliated | RP4-666F24.3 |
| PKIG | 0.654 | 0.886513527 | 0.308 | 0.343 | 0.038 | Ciliated | PKIG |
| ANKRD66 | 0.653 | 1.048352002 | 0.306 | 0.307 | 0 | Ciliated | ANKRD66 |
| C9orf9 | 0.653 | 0.910176115 | 0.306 | 0.315 | 0.01 | Ciliated | C9orf9 |
| SYAP1 | 0.653 | 0.751738588 | 0.306 | 0.502 | 0.216 | Ciliated | SYAP1 |
| TMBIM6 | 0.653 | 0.447906435 | 0.306 | 0.797 | 0.608 | Ciliated | TMBIM6 |
| C21orf58 | 0.652 | 1.048411437 | 0.304 | 0.313 | 0.01 | Ciliated | C21orf58 |
| PCDP1 | 0.652 | 0.985559863 | 0.304 | 0.305 | 0.001 | Ciliated | PCDP1 |
| TUSC3 | 0.652 | 0.84261495 | 0.304 | 0.376 | 0.075 | Ciliated | TUSC3 |
| UBL5 | 0.652 | 0.536469382 | 0.304 | 0.715 | 0.509 | Ciliated | UBL5 |
| LYZ1 | 0.97 | 4.464037689 | 0.94 | 0.969 | 0.309 | Glandular | LYZ |
| AZGP12 | 0.951 | 3.246710682 | 0.902 | 0.911 | 0.061 | Glandular | AZGP1 |
| LTF2 | 0.945 | 3.934203652 | 0.89 | 0.905 | 0.111 | Glandular | LTF |
| ZG16B1 | 0.94 | 4.168626246 | 0.88 | 0.913 | 0.207 | Glandular | ZG16B |
| STATH1 | 0.932 | 4.762796252 | 0.864 | 0.946 | 0.434 | Glandular | STATH |
| TCN11 | 0.929 | 2.843768864 | 0.858 | 0.881 | 0.09 | Glandular | TCN1 |
| BPIFB11 | 0.926 | 2.284273017 | 0.852 | 0.968 | 0.436 | Glandular | BPIFB1 |
| PIGR1 | 0.924 | 1.77340537 | 0.848 | 0.974 | 0.414 | Glandular | PIGR |
| SLPI1 | 0.915 | 1.673805968 | 0.83 | 1 | 0.931 | Glandular | SLPI |
| BPIFA11 | 0.906 | 3.146682133 | 0.812 | 0.956 | 0.506 | Glandular | BPIFA1 |
| PIP | 0.875 | 3.489356595 | 0.75 | 0.769 | 0.052 | Glandular | PIP |
| C6orf58 | 0.874 | 3.808025136 | 0.748 | 0.764 | 0.051 | Glandular | C6orf58 |
| DMBT1 | 0.874 | 2.86069129 | 0.748 | 0.76 | 0.036 | Glandular | DMBT1 |
| RP11-1143G9.4 | 0.861 | 3.70853547 | 0.722 | 0.745 | 0.055 | Glandular | RP11-1143G9.4 |
| ODAM | 0.822 | 2.439899892 | 0.644 | 0.663 | 0.038 | Glandular | ODAM |
| XBP11 | 0.783 | 1.02943328 | 0.566 | 0.841 | 0.442 | Glandular | XBP1 |
| RNASE1 | 0.781 | 1.981469286 | 0.562 | 0.58 | 0.026 | Glandular | RNASE1 |
| NUCB22 | 0.735 | 0.973445063 | 0.47 | 0.662 | 0.228 | Glandular | NUCB2 |
| CCL28 | 0.734 | 1.362722356 | 0.468 | 0.497 | 0.033 | Glandular | CCL28 |
| SEC11C | 0.724 | 1.005032406 | 0.448 | 0.613 | 0.205 | Glandular | SEC11C |
| SSR4 | 0.701 | 0.701060339 | 0.402 | 0.759 | 0.484 | Glandular | SSR4 |
| SCGB3A1 | 0.7 | 2.544462805 | 0.4 | 0.474 | 0.089 | Glandular | SCGB3A1 |
| NDRG2 | 0.699 | 0.916065595 | 0.398 | 0.552 | 0.176 | Glandular | NDRG2 |
| CA2 | 0.698 | 1.216953259 | 0.396 | 0.416 | 0.021 | Glandular | CA2 |
| PHLDA1 | 0.694 | 1.003051242 | 0.388 | 0.479 | 0.094 | Glandular | PHLDA1 |
| CST31 | 0.688 | 0.678097334 | 0.376 | 0.725 | 0.42 | Glandular | CST3 |
| CXCL171 | 0.688 | 0.612963877 | 0.376 | 0.764 | 0.449 | Glandular | CXCL17 |
| LRRC26 | 0.683 | 1.010633591 | 0.366 | 0.402 | 0.036 | Glandular | LRRC26 |
| SLC12A2 | 0.683 | 0.872412255 | 0.366 | 0.56 | 0.213 | Glandular | SLC12A2 |
| PPP1R1B | 0.675 | 1.023949812 | 0.35 | 0.354 | 0.005 | Glandular | PPP1R1B |
| PART | 0.669 | 0.876720153 | 0.338 | 0.397 | 0.061 | Glandular | PART |
| TMED3 | 0.665 | 0.733483511 | 0.33 | 0.511 | 0.196 | Glandular | TMED3 |
| FDCSP | 0.66 | 2.976897006 | 0.32 | 0.354 | 0.046 | Glandular | FDCSP |
| FAM3D | 0.66 | 0.729255542 | 0.32 | 0.482 | 0.163 | Glandular | FAM3D |
| PRR4 | 0.651 | 2.110747723 | 0.302 | 0.338 | 0.041 | Glandular | PRR4 |
| HP | 0.651 | 1.616772262 | 0.302 | 0.309 | 0.008 | Glandular | HP |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 13. The clusters are as shown in FIG. 2C. The biomarkers listed in Table 13 may be used to differentiated basal and secretory genes that are enriched in polyps/severe allergic inflammation (clusters 12, 2, 0) or less severe (clusters 8, 1, 4).

TABLE 13

| | myAUC | avg_diff | power | pct. 1 | pct. 2 | cluster | gene |
|---|---|---|---|---|---|---|---|
| Basal | | | | | | | |
| POSTN | 0.737 | 0.604151475 | 0.474 | 0.991 | 0.735 | 12 | POSTN |
| ALOX15 | 0.724 | 0.613027437 | 0.448 | 0.935 | 0.647 | 12 | ALOX15 |
| S100A2 | 0.684 | 0.537505975 | 0.368 | 0.969 | 0.848 | 12 | S100A2 |
| PTHLH | 0.683 | 0.537570937 | 0.366 | 0.665 | 0.256 | 12 | PTHLH |
| NTRK2 | 0.661 | 0.605220865 | 0.322 | 0.519 | 0.177 | 12 | NTRK2 |
| MMP10 | 0.655 | 0.524609303 | 0.31 | 0.787 | 0.518 | 12 | MMP10 |
| CTSC | 0.648 | 0.461571495 | 0.296 | 0.613 | 0.304 | 12 | CTSC |
| CD55 | 0.647 | 0.429784607 | 0.294 | 0.713 | 0.423 | 12 | CD55 |
| CD9 | 0.647 | 0.303592797 | 0.294 | 0.958 | 0.806 | 12 | CD9 |
| CD44 | 0.642 | 0.351010854 | 0.284 | 0.719 | 0.409 | 12 | CD44 |
| HS3ST1 | 0.64 | 0.43626783 | 0.28 | 0.563 | 0.266 | 12 | HS3ST1 |
| TXNDC17 | 0.639 | 0.41023641 | 0.278 | 0.651 | 0.366 | 12 | TXNDC17 |
| TACSTD2 | 0.639 | 0.322263939 | 0.278 | 0.94 | 0.769 | 12 | TACSTD2 |
| SERPINB10 | 0.635 | 0.649821507 | 0.27 | 0.356 | 0.074 | 12 | SERPINB10 |
| SERPINB2 | 0.634 | 0.905259547 | 0.268 | 0.364 | 0.088 | 12 | SERPINB2 |
| SFN | 0.634 | 0.410698336 | 0.268 | 0.63 | 0.35 | 12 | SFN |
| MIF | 0.629 | 0.34649737 | 0.258 | 0.568 | 0.286 | 12 | MIF |
| ANXA2 | 0.626 | 0.344503845 | 0.252 | 0.872 | 0.657 | 12 | ANXA2 |
| CDH26 | 0.623 | 0.47273847 | 0.246 | 0.36 | 0.099 | 12 | CDH26 |
| IGJ | 0.623 | 0.278754038 | 0.246 | 0.63 | 0.361 | 12 | IGJ |
| TNC | 0.62 | 0.314929278 | 0.24 | 0.699 | 0.443 | 12 | TNC |
| TMSB4X | 0.62 | 0.279040394 | 0.24 | 0.772 | 0.53 | 12 | TMSB4X |
| GPX4 | 0.619 | 0.282456586 | 0.238 | 0.836 | 0.605 | 12 | GPX4 |
| LGALS3 | 0.617 | 0.291574168 | 0.234 | 0.81 | 0.596 | 12 | LGALS3 |
| KCNJ16 | 0.616 | 0.623690554 | 0.232 | 0.292 | 0.051 | 12 | KCNJ16 |
| S100A10 | 0.613 | 0.330998377 | 0.226 | 0.651 | 0.416 | 12 | S100A10 |
| MYL12B | 0.612 | 0.2532763 | 0.224 | 0.835 | 0.624 | 12 | MYL12B |
| KRT17 | 0.611 | 0.302912902 | 0.222 | 0.7 | 0.469 | 12 | KRT17 |
| MRPS6 | 0.61 | 0.331906917 | 0.22 | 0.436 | 0.198 | 12 | MRPS6 |
| SERPINB3 | 0.61 | 0.257947406 | 0.22 | 0.831 | 0.573 | 12 | SERPINB3 |
| SERPINB4 | 0.609 | 0.431394584 | 0.218 | 0.55 | 0.318 | 12 | SERPINB4 |
| LGALS7 | 0.606 | 0.449181475 | 0.212 | 0.384 | 0.16 | 12 | LGALS7 |
| LDHA | 0.605 | 0.250695992 | 0.21 | 0.601 | 0.355 | 12 | LDHA |
| GPR155 | 0.604 | 0.423893675 | 0.208 | 0.365 | 0.145 | 12 | GPR155 |
| KRT6A | 0.603 | 0.495409386 | 0.206 | 0.384 | 0.173 | 12 | KRT6A |
| CBR1 | 0.603 | 0.275996439 | 0.206 | 0.573 | 0.346 | 12 | CBR1 |
| CCL26 | 0.601 | 0.770295793 | 0.202 | 0.293 | 0.086 | 12 | CCL26 |
| STATH1 | 0.882 | 2.131487578 | 0.764 | 0.816 | 0.055 | 8 | STATH |
| EPAS1 | 0.777 | 1.019217535 | 0.554 | 0.811 | 0.307 | 8 | EPAS1 |
| BPIFA1 | 0.749 | 1.069532139 | 0.498 | 0.679 | 0.177 | 8 | BPIFA1 |
| LYZ | 0.737 | 1.294945728 | 0.474 | 0.515 | 0.038 | 8 | LYZ |
| BPIFB1 | 0.709 | 0.850152967 | 0.418 | 0.537 | 0.102 | 8 | BPIFB1 |
| GLUL | 0.696 | 0.506042005 | 0.392 | 0.883 | 0.508 | 8 | GLUL |
| SPINK5 | 0.695 | 1.174528977 | 0.39 | 0.51 | 0.12 | 8 | SPINK5 |
| AGR2 | 0.688 | 0.508444077 | 0.376 | 0.806 | 0.405 | 8 | AGR2 |
| ZG16B | 0.687 | 0.970203699 | 0.374 | 0.4 | 0.022 | 8 | ZG16B |
| ALDH3A1 | 0.683 | 0.583955015 | 0.366 | 0.793 | 0.423 | 8 | ALDH3A1 |
| AQP3 | 0.672 | 0.447709571 | 0.344 | 0.946 | 0.726 | 8 | AQP3 |
| CTSB | 0.669 | 0.447799473 | 0.338 | 0.763 | 0.421 | 8 | CTSB |
| MT1X | 0.669 | 0.370895907 | 0.338 | 0.786 | 0.419 | 8 | MT1X |
| ID2 | 0.66 | 0.49344398 | 0.32 | 0.606 | 0.258 | 8 | ID2 |
| TSC22D1 | 0.66 | 0.447125468 | 0.32 | 0.868 | 0.547 | 8 | TSC22D1 |
| ZFP36L1 | 0.659 | 0.379647301 | 0.318 | 0.884 | 0.565 | 8 | ZFP36L1 |
| KRT19 | 0.656 | 0.331342528 | 0.312 | 0.972 | 0.859 | 8 | KRT19 |
| RPS4X | 0.655 | 0.314176463 | 0.31 | 0.974 | 0.863 | 8 | RPS4X |
| XIST | 0.651 | 0.521737838 | 0.302 | 0.504 | 0.174 | 8 | XIST |
| DDIT4 | 0.651 | 0.467567374 | 0.302 | 0.531 | 0.199 | 8 | DDIT4 |
| CSRP2 | 0.65 | 0.687316828 | 0.3 | 0.378 | 0.073 | 8 | CSRP2 |
| FAM107A | 0.647 | 0.644945445 | 0.294 | 0.361 | 0.06 | 8 | FAM107A |
| EGFR | 0.647 | 0.435017225 | 0.294 | 0.543 | 0.215 | 8 | EGFR |
| AKR1C3 | 0.646 | 0.772661721 | 0.292 | 0.351 | 0.056 | 8 | AKR1C3 |
| ZFP36L2 | 0.646 | 0.374537312 | 0.292 | 0.71 | 0.366 | 8 | ZFP36L2 |
| CLU | 0.643 | 0.359737197 | 0.286 | 0.58 | 0.25 | 8 | CLU |
| PRDX6 | 0.641 | 0.379839709 | 0.282 | 0.525 | 0.211 | 8 | PRDX6 |
| TOB1 | 0.64 | 0.360023324 | 0.28 | 0.657 | 0.334 | 8 | TOB1 |
| S100A4 | 0.639 | 0.664953103 | 0.278 | 0.412 | 0.123 | 8 | S100A4 |
| NET1 | 0.639 | 0.507864178 | 0.278 | 0.424 | 0.13 | 8 | NET1 |
| HLA-A | 0.638 | 0.383167684 | 0.276 | 0.768 | 0.486 | 8 | HLA-A |
| CFH | 0.638 | 0.358267996 | 0.276 | 0.527 | 0.214 | 8 | CFH |
| DUSP1 | 0.637 | 0.378941573 | 0.274 | 0.921 | 0.749 | 8 | DUSP1 |
| MT2A | 0.637 | 0.320859757 | 0.274 | 0.562 | 0.255 | 8 | MT2A |
| ACAP2 | 0.636 | 0.420400876 | 0.272 | 0.46 | 0.164 | 8 | ACAP2 |
| ALDH2 | 0.636 | 0.390633877 | 0.272 | 0.464 | 0.167 | 8 | ALDH2 |
| CA12 | 0.635 | 0.570131253 | 0.27 | 0.363 | 0.082 | 8 | CA12 |
| UGT2A2 | 0.635 | 0.430783305 | 0.27 | 0.607 | 0.31 | 8 | UGT2A2 |
| KRT8 | 0.634 | 0.446871862 | 0.268 | 0.662 | 0.384 | 8 | KRT8 |
| ALDH1A1 | 0.632 | 0.33568301 | 0.264 | 0.648 | 0.34 | 8 | ALDH1A1 |
| FMO3 | 0.63 | 0.478030885 | 0.26 | 0.434 | 0.153 | 8 | FMO3 |
| ANXA1 | 0.63 | 0.362409652 | 0.26 | 0.918 | 0.701 | 8 | ANXA1 |
| CLK1 | 0.629 | 0.309236789 | 0.258 | 0.504 | 0.208 | 8 | CLK1 |
| PIGR | 0.628 | 0.735577276 | 0.256 | 0.305 | 0.045 | 8 | PIGR |
| PTN | 0.628 | 0.58342652 | 0.256 | 0.318 | 0.057 | 8 | PTN |
| ALCAM | 0.628 | 0.361698016 | 0.256 | 0.511 | 0.221 | 8 | ALCAM |
| PIK3R1 | 0.628 | 0.355257122 | 0.256 | 0.522 | 0.23 | 8 | PIK3R1 |
| CCND1 | 0.627 | 0.326060892 | 0.254 | 0.474 | 0.188 | 8 | CCND1 |
| PSAP | 0.627 | 0.281151177 | 0.254 | 0.715 | 0.388 | 8 | PSAP |
| EXPH5 | 0.625 | 0.404390443 | 0.25 | 0.402 | 0.133 | 8 | EXPH5 |
| TXNIP | 0.625 | 0.272513635 | 0.25 | 0.84 | 0.547 | 8 | TXNIP |
| NFIB | 0.623 | 0.289874809 | 0.246 | 0.517 | 0.229 | 8 | NFIB |
| SNHG8 | 0.623 | 0.253443755 | 0.246 | 0.652 | 0.344 | 8 | SNHG8 |
| CYP4B1 | 0.621 | 0.446483909 | 0.242 | 0.392 | 0.134 | 8 | CYP4B1 |
| TOMM7 | 0.621 | 0.258727845 | 0.242 | 0.669 | 0.37 | 8 | TOMM7 |
| AQP5 | 0.62 | 0.377247243 | 0.24 | 0.387 | 0.131 | 8 | AQP5 |
| MKL2 | 0.619 | 0.356795504 | 0.238 | 0.363 | 0.108 | 8 | MKL2 |
| WFDC2 | 0.618 | 0.332914301 | 0.236 | 0.558 | 0.288 | 8 | WFDC2 |
| RTN4 | 0.618 | 0.306117753 | 0.236 | 0.442 | 0.176 | 8 | RTN4 |
| CAST | 0.618 | 0.275386981 | 0.236 | 0.663 | 0.363 | 8 | CAST |
| SULT1E1 | 0.616 | 0.695072054 | 0.232 | 0.29 | 0.054 | 8 | SULT1E1 |
| ITGA2 | 0.616 | 0.312910743 | 0.232 | 0.417 | 0.16 | 8 | ITGA2 |
| DUSP6 | 0.616 | 0.30116556 | 0.232 | 0.437 | 0.176 | 8 | DUSP6 |
| SAT1 | 0.616 | 0.287574025 | 0.232 | 0.846 | 0.625 | 8 | SAT1 |
| LTF | 0.615 | 0.609403527 | 0.23 | 0.242 | 0.012 | 8 | LTF |
| DUSP2 | 0.615 | 0.401324844 | 0.23 | 0.41 | 0.161 | 8 | DUSP2 |
| CD74 | 0.614 | 0.434586642 | 0.228 | 0.46 | 0.215 | 8 | CD74 |
| HES1 | 0.614 | 0.303532111 | 0.228 | 0.71 | 0.437 | 8 | HES1 |
| PBX1 | 0.611 | 0.332701385 | 0.222 | 0.339 | 0.101 | 8 | PBX1 |
| KIF21A | 0.611 | 0.297086615 | 0.222 | 0.348 | 0.11 | 8 | KIF21A |
| KRT7 | 0.61 | 0.417043001 | 0.22 | 0.324 | 0.092 | 8 | KRT7 |
| CITED2 | 0.61 | 0.413969047 | 0.22 | 0.344 | 0.112 | 8 | CITED2 |
| HSPB1 | 0.61 | 0.281544995 | 0.22 | 0.786 | 0.531 | 8 | HSPB1 |
| CXCL17 | 0.609 | 0.299032527 | 0.218 | 0.405 | 0.165 | 8 | CXCL17 |
| HLA-B | 0.608 | 0.3037082 | 0.216 | 0.89 | 0.709 | 8 | HLA-B |
| WSB1 | 0.608 | 0.2527087 | 0.216 | 0.42 | 0.175 | 8 | WSB1 |
| PTPN13 | 0.607 | 0.262978661 | 0.214 | 0.404 | 0.164 | 8 | PTPN13 |
| IL18 | 0.606 | 0.392572878 | 0.212 | 0.293 | 0.071 | 8 | IL18 |
| GDI2 | 0.606 | 0.273753816 | 0.212 | 0.382 | 0.148 | 8 | GDI2 |
| FN1 | 0.605 | 1.026058616 | 0.21 | 0.226 | 0.016 | 8 | FN1 |
| NFIA | 0.604 | 0.289182401 | 0.208 | 0.36 | 0.134 | 8 | NFIA |
| EPB41L4A-AS1 | 0.604 | 0.272172921 | 0.208 | 0.36 | 0.133 | 8 | EPB41L4A-AS1 |
| DDIT3 | 0.604 | 0.266989252 | 0.208 | 0.377 | 0.147 | 8 | DDIT3 |
| MSMB | 0.603 | 0.441153002 | 0.206 | 0.28 | 0.066 | 8 | MSMB |
| SCGB1A1 | 0.602 | 0.998144583 | 0.204 | 0.226 | 0.021 | 8 | SCGB1A1 |
| OGFRL1 | 0.602 | 0.398131116 | 0.204 | 0.286 | 0.074 | 8 | OGFRL1 |
| IFI27 | 0.602 | 0.35444363 | 0.204 | 0.41 | 0.189 | 8 | IFI27 |
| TMPRSS11D | 0.601 | 0.438722209 | 0.202 | 0.253 | 0.047 | 8 | TMPRSS11D |
| CD82 | 0.601 | 0.287839385 | 0.202 | 0.337 | 0.118 | 8 | CD82 |
| MTRNR2L11 | 0.723 | 0.930719706 | 0.446 | 0.892 | 0.836 | 2 | MTRNR2L1 |
| POSTN2 | 0.64 | 0.406928518 | 0.28 | 0.882 | 0.725 | 2 | POSTN |
| Apical | | | | | | | |
| STATH | 0.686 | 0.78707747 | 0.372 | 0.744 | 0.408 | 1 | STATH |
| EPAS1 | 0.681 | 0.642405774 | 0.362 | 0.778 | 0.592 | 1 | EPAS1 |
| AQP3 | 0.672 | 0.584397457 | 0.344 | 0.846 | 0.726 | 1 | AQP3 |
| EGR1 | 0.667 | 0.539905387 | 0.334 | 0.851 | 0.737 | 1 | EGR1 |
| KRT5 | 0.661 | 0.99641558 | 0.322 | 0.507 | 0.224 | 1 | KRT5 |
| JUNB | 0.633 | 0.412468099 | 0.266 | 0.855 | 0.779 | 1 | JUNB |
| TSC22D1 | 0.625 | 0.5662689 | 0.25 | 0.619 | 0.468 | 1 | TSC22D1 |
| GLUL | 0.617 | 0.463018268 | 0.234 | 0.703 | 0.58 | 1 | GLUL |
| PERP | 0.614 | 0.342349213 | 0.228 | 0.831 | 0.774 | 1 | PERP |
| ALDH3A1 | 0.609 | 0.510680701 | 0.218 | 0.559 | 0.382 | 1 | ALDH3A1 |
| EGFR | 0.606 | 0.742932266 | 0.212 | 0.349 | 0.16 | 1 | EGFR |
| TXNIP | 0.604 | 0.439601783 | 0.208 | 0.652 | 0.543 | 1 | TXNIP |
| CST11 | 0.862 | 2.381869218 | 0.724 | 0.811 | 0.145 | 0 | CST1 |
| ALOX151 | 0.785 | 1.146991722 | 0.57 | 0.834 | 0.583 | 0 | ALOX15 |
| SERPINB31 | 0.771 | 0.753672912 | 0.542 | 0.984 | 0.843 | 0 | SERPINB3 |
| IGJ1 | 0.763 | 1.615027828 | 0.526 | 0.648 | 0.196 | 0 | IGJ |
| CST41 | 0.762 | 2.61405295 | 0.524 | 0.564 | 0.062 | 0 | CST4 |
| POSTN | 0.744 | 1.826029118 | 0.488 | 0.603 | 0.158 | 0 | POSTN |
| PTHLH1 | 0.74 | 1.87541583 | 0.48 | 0.516 | 0.046 | 0 | PTHLH |
| IGHA11 | 0.738 | 1.39553703 | 0.476 | 0.678 | 0.35 | 0 | IGHA1 |
| IGFBP31 | 0.718 | 1.456877504 | 0.436 | 0.597 | 0.237 | 0 | IGFBP3 |
| RPL41 | 0.692 | 0.525625495 | 0.384 | 0.901 | 0.85 | 0 | RPL41 |
| MTRNR2L1 | 0.692 | 0.424063132 | 0.384 | 0.94 | 0.802 | 0 | MTRNR2L1 |
| SERPINB4 | 0.687 | 0.806842966 | 0.374 | 0.732 | 0.506 | 0 | SERPINB4 |
| CST21 | 0.681 | 1.842440407 | 0.362 | 0.382 | 0.024 | 0 | CST2 |
| RPL13A | 0.668 | 0.413945206 | 0.336 | 0.929 | 0.857 | 0 | RPL13A |
| TFF31 | 0.663 | 1.420422635 | 0.326 | 0.569 | 0.299 | 0 | TFF3 |
| EGLN31 | 0.662 | 1.373569421 | 0.324 | 0.361 | 0.047 | 0 | EGLN3 |
| IGHG4 | 0.657 | 1.923369017 | 0.314 | 0.352 | 0.047 | 0 | IGHG4 |
| CDH261 | 0.653 | 1.119394839 | 0.306 | 0.377 | 0.082 | 0 | CDH26 |
| HS3ST11 | 0.648 | 0.833935735 | 0.296 | 0.557 | 0.356 | 0 | HS3ST1 |
| MIF | 0.624 | 0.810454117 | 0.248 | 0.442 | 0.252 | 0 | MIF |
| RPL36 | 0.621 | 0.415379073 | 0.242 | 0.735 | 0.631 | 0 | RPL36 |
| IGHA2 | 0.62 | 1.150938172 | 0.24 | 0.307 | 0.073 | 0 | IGHA2 |
| RPL10 | 0.62 | 0.484542201 | 0.24 | 0.68 | 0.554 | 0 | RPL10 |
| RPL18A | 0.619 | 0.64255482 | 0.238 | 0.55 | 0.404 | 0 | RPL18A |
| RPS27 | 0.615 | 0.499116627 | 0.23 | 0.648 | 0.509 | 0 | RPS27 |
| MTRNR2L2 | 0.608 | 0.449669615 | 0.216 | 0.788 | 0.697 | 0 | MTRNR2L2 |
| SLC6A141 | 0.607 | 0.727637276 | 0.214 | 0.466 | 0.305 | 0 | SLC6A14 |
| GSN1 | 0.605 | 0.673421461 | 0.21 | 0.525 | 0.404 | 0 | GSN |
| MT-CO2 | 0.602 | 0.507360096 | 0.204 | 0.717 | 0.653 | 0 | MT-CO2 |
| MTRNR2L3 | 0.601 | 0.482721912 | 0.202 | 0.604 | 0.485 | 0 | MTRNR2L3 |
| WFDC21 | 0.873 | 1.222705433 | 0.746 | 0.991 | 0.706 | 4 | WFDC2 |
| PIGR1 | 0.861 | 1.268598878 | 0.722 | 0.975 | 0.443 | 4 | PIGR |
| LYPD22 | 0.821 | 1.905743568 | 0.642 | 0.731 | 0.114 | 4 | LYPD2 |
| PSCA2 | 0.808 | 1.86002051 | 0.616 | 0.706 | 0.108 | 4 | PSCA |
| MSMB1 | 0.794 | 2.518337255 | 0.588 | 0.751 | 0.233 | 4 | MSMB |
| VMO11 | 0.784 | 1.037035043 | 0.568 | 0.92 | 0.52 | 4 | VMO1 |
| BPIFB11 | 0.784 | 0.987216981 | 0.568 | 0.905 | 0.44 | 4 | BPIFB1 |
| AGR21 | 0.774 | 0.86314702 | 0.548 | 0.993 | 0.746 | 4 | AGR2 |
| SCGB1A11 | 0.766 | 2.105107943 | 0.532 | 0.699 | 0.217 | 4 | SCGB1A1 |
| S100A41 | 0.743 | 1.005373902 | 0.486 | 0.803 | 0.401 | 4 | S100A4 |
| CP1 | 0.735 | 0.822171588 | 0.47 | 0.829 | 0.433 | 4 | CP |
| RARRES11 | 0.731 | 1.18901352 | 0.462 | 0.631 | 0.174 | 4 | RARRES1 |
| STATH2 | 0.721 | 0.551486148 | 0.442 | 0.858 | 0.403 | 4 | STATH |
| LCN21 | 0.72 | 1.293391974 | 0.44 | 0.613 | 0.19 | 4 | LCN2 |
| CXCL171 | 0.714 | 0.618148444 | 0.428 | 0.874 | 0.513 | 4 | CXCL17 |
| BPIFA1 | 0.713 | 1.250209713 | 0.426 | 0.87 | 0.531 | 4 | BPIFA1 |
| CST31 | 0.711 | 0.68382289 | 0.422 | 0.784 | 0.385 | 4 | CST3 |
| STEAP41 | 0.71 | 1.211299133 | 0.42 | 0.473 | 0.053 | 4 | STEAP4 |
| S100P1 | 0.709 | 0.835786801 | 0.418 | 0.75 | 0.361 | 4 | S100P |
| FAM3D1 | 0.7 | 0.840215453 | 0.4 | 0.55 | 0.146 | 4 | FAM3D |
| PI31 | 0.699 | 1.081402118 | 0.398 | 0.48 | 0.084 | 4 | PI3 |
| LYZ1 | 0.699 | 0.814298306 | 0.398 | 0.677 | 0.271 | 4 | LYZ |
| LGALS31 | 0.696 | 0.492987709 | 0.392 | 0.944 | 0.78 | 4 | LGALS3 |
| HLA-B1 | 0.687 | 0.538860228 | 0.374 | 0.943 | 0.791 | 4 | HLA-B |
| IFI27 | 0.683 | 0.793525521 | 0.366 | 0.65 | 0.311 | 4 | IFI27 |
| TSPAN11 | 0.681 | 0.500724805 | 0.362 | 0.857 | 0.52 | 4 | TSPAN1 |
| CD74 | 0.677 | 0.805013584 | 0.354 | 0.639 | 0.313 | 4 | CD74 |
| MSLN1 | 0.674 | 0.893742178 | 0.348 | 0.482 | 0.134 | 4 | MSLN |
| KRT7 | 0.673 | 0.526974985 | 0.346 | 0.747 | 0.401 | 4 | KRT7 |
| ALCAM | 0.673 | 0.482740084 | 0.346 | 0.816 | 0.474 | 4 | ALCAM |
| NUCB2 | 0.672 | 0.727065698 | 0.344 | 0.505 | 0.153 | 4 | NUCB2 |
| XBP11 | 0.669 | 0.547283212 | 0.338 | 0.802 | 0.457 | 4 | XBP1 |
| ALDH1A11 | 0.665 | 0.436820022 | 0.33 | 0.875 | 0.584 | 4 | ALDH1A1 |
| SLC31A11 | 0.662 | 0.498016903 | 0.324 | 0.59 | 0.245 | 4 | SLC31A1 |
| PRSS231 | 0.661 | 0.368910192 | 0.322 | 0.933 | 0.757 | 4 | PRSS23 |
| HLA-C | 0.658 | 0.522079842 | 0.316 | 0.823 | 0.582 | 4 | HLA-C |
| TSPAN8 | 0.653 | 0.86747323 | 0.306 | 0.375 | 0.064 | 4 | TSPAN8 |
| TMEM2131 | 0.652 | 0.597171751 | 0.304 | 0.487 | 0.167 | 4 | TMEM213 |
| MUC16 | 0.651 | 0.715037055 | 0.302 | 0.421 | 0.112 | 4 | MUC16 |
| HLA-DRA | 0.649 | 0.764354382 | 0.298 | 0.47 | 0.171 | 4 | HLA-DRA |
| CLU1 | 0.648 | 0.617980022 | 0.296 | 0.535 | 0.227 | 4 | CLU |
| SORD | 0.647 | 0.584954353 | 0.294 | 0.482 | 0.177 | 4 | SORD |
| LY6E | 0.647 | 0.463683611 | 0.294 | 0.58 | 0.273 | 4 | LY6E |
| SCGB3A1 | 0.646 | 1.596108903 | 0.292 | 0.331 | 0.043 | 4 | SCGB3A1 |
| CYP2A13 | 0.646 | 0.953460249 | 0.292 | 0.345 | 0.051 | 4 | CYP2A13 |
| ZG16B1 | 0.645 | 0.507805833 | 0.29 | 0.475 | 0.162 | 4 | ZG16B |
| GDF15 | 0.642 | 0.684387212 | 0.284 | 0.49 | 0.2 | 4 | GDF15 |
| S100A13 | 0.642 | 0.471411256 | 0.284 | 0.562 | 0.258 | 4 | S100A13 |
| POR | 0.641 | 0.445255106 | 0.282 | 0.565 | 0.252 | 4 | POR |
| CTD-2319I12.11 | 0.64 | 0.47881043 | 0.28 | 0.587 | 0.292 | 4 | CTD-2319I12.1 |
| DHRS9 | 0.639 | 0.645540283 | 0.278 | 0.376 | 0.091 | 4 | DHRS9 |
| HLA-A1 | 0.639 | 0.440829309 | 0.278 | 0.814 | 0.58 | 4 | HLA-A |
| PLAC8 | 0.639 | 0.423983314 | 0.278 | 0.517 | 0.212 | 4 | PLAC8 |
| CYB5A | 0.637 | 0.405354056 | 0.274 | 0.611 | 0.315 | 4 | CYB5A |
| S100A14 | 0.636 | 0.462466019 | 0.272 | 0.496 | 0.208 | 4 | S100A14 |
| CYP2F1 | 0.635 | 0.932254073 | 0.27 | 0.318 | 0.047 | 4 | CYP2F1 |
| TMEM59 | 0.635 | 0.342925848 | 0.27 | 0.793 | 0.513 | 4 | TMEM59 |
| ASAH1 | 0.634 | 0.370721147 | 0.268 | 0.595 | 0.293 | 4 | ASAH1 |
| RARRES3 | 0.631 | 0.587942893 | 0.262 | 0.389 | 0.12 | 4 | RARRES3 |
| RIMS1 | 0.629 | 0.619954507 | 0.258 | 0.415 | 0.15 | 4 | RIMS1 |
| ASS1 | 0.629 | 0.519928009 | 0.258 | 0.462 | 0.194 | 4 | ASS1 |
| MUC1 | 0.629 | 0.511480299 | 0.258 | 0.426 | 0.154 | 4 | MUC1 |
| MMP10 | 0.628 | 0.926584867 | 0.256 | 0.42 | 0.165 | 4 | MMP10 |
| CD59 | 0.628 | 0.36034083 | 0.256 | 0.649 | 0.36 | 4 | CD59 |
| GSTA1 | 0.627 | 0.905518474 | 0.254 | 0.288 | 0.034 | 4 | GSTA1 |
| AQP51 | 0.627 | 0.432794598 | 0.254 | 0.565 | 0.288 | 4 | AQP5 |
| CYP4B11 | 0.626 | 0.426832197 | 0.252 | 0.575 | 0.295 | 4 | CYP4B1 |
| RDH10 | 0.625 | 0.462583058 | 0.25 | 0.465 | 0.197 | 4 | RDH10 |
| TNFSF10 | 0.624 | 0.353862542 | 0.248 | 0.698 | 0.433 | 4 | TNFSF10 |
| ANXA5 | 0.624 | 0.334325073 | 0.248 | 0.635 | 0.351 | 4 | ANXA5 |
| CRYM | 0.622 | 0.680533181 | 0.244 | 0.293 | 0.047 | 4 | CRYM |
| HLA-DRB1 | 0.622 | 0.678160435 | 0.244 | 0.346 | 0.1 | 4 | HLA-DRB1 |
| C8orf4 | 0.622 | 0.474382595 | 0.244 | 0.523 | 0.27 | 4 | C8orf4 |
| MDK | 0.622 | 0.346017486 | 0.244 | 0.639 | 0.366 | 4 | MDK |
| CLDN71 | 0.622 | 0.325049691 | 0.244 | 0.634 | 0.358 | 4 | CLDN7 |
| SCGB2A1 | 0.621 | 0.722295642 | 0.242 | 0.316 | 0.071 | 4 | SCGB2A1 |
| GOLM1 | 0.621 | 0.474796606 | 0.242 | 0.341 | 0.091 | 4 | GOLM1 |
| SPINT2 | 0.62 | 0.290091948 | 0.24 | 0.809 | 0.538 | 4 | SPINT2 |
| RTN3 | 0.618 | 0.305688271 | 0.236 | 0.529 | 0.263 | 4 | RTN3 |
| CD63 | 0.617 | 0.28836323 | 0.234 | 0.837 | 0.592 | 4 | CD63 |
| FAM129A | 0.616 | 0.428282883 | 0.232 | 0.391 | 0.145 | 4 | FAM129A |
| SELENBP1 | 0.616 | 0.375661874 | 0.232 | 0.451 | 0.199 | 4 | SELENBP1 |
| MUC5AC | 0.615 | 1.459417226 | 0.23 | 0.334 | 0.109 | 4 | MUC5AC |
| C3 | 0.614 | 0.598133035 | 0.228 | 0.316 | 0.086 | 4 | C3 |
| IDH1 | 0.614 | 0.457133528 | 0.228 | 0.383 | 0.145 | 4 | IDH1 |
| DHCR24 | 0.614 | 0.309469309 | 0.228 | 0.51 | 0.246 | 4 | DHCR24 |
| CD164 | 0.611 | 0.286760951 | 0.222 | 0.561 | 0.299 | 4 | CD164 |
| SERP1 | 0.611 | 0.270061453 | 0.222 | 0.609 | 0.341 | 4 | SERP1 |
| HLA-E | 0.61 | 0.298962598 | 0.22 | 0.737 | 0.509 | 4 | HLA-E |
| CYP2J2 | 0.609 | 0.512043903 | 0.218 | 0.332 | 0.105 | 4 | CYP2J2 |
| UGT2A21 | 0.609 | 0.277814169 | 0.218 | 0.759 | 0.512 | 4 | UGT2A2 |
| RHOA | 0.609 | 0.276702969 | 0.218 | 0.704 | 0.442 | 4 | RHOA |
| PDIA3 | 0.609 | 0.266274921 | 0.218 | 0.643 | 0.388 | 4 | PDIA3 |
| ABLIM1 | 0.608 | 0.373813979 | 0.216 | 0.366 | 0.133 | 4 | ABLIM1 |
| TMC5 | 0.607 | 0.488660677 | 0.214 | 0.299 | 0.079 | 4 | TMC5 |
| ALDH3A12 | 0.607 | 0.395393678 | 0.214 | 0.609 | 0.383 | 4 | ALDH3A1 |
| TMEM66 | 0.607 | 0.262628659 | 0.214 | 0.565 | 0.31 | 4 | TMEM66 |
| CD551 | 0.607 | 0.259962794 | 0.214 | 0.782 | 0.541 | 4 | CD55 |
| CD82 | 0.606 | 0.27743365 | 0.212 | 0.489 | 0.245 | 4 | CD82 |
| ST6GALNAC1 | 0.606 | 0.262365077 | 0.212 | 0.494 | 0.245 | 4 | ST6GALNAC1 |
| EFCAB4A | 0.604 | 0.432657012 | 0.208 | 0.311 | 0.095 | 4 | EFCAB4A |
| OAT | 0.604 | 0.266065353 | 0.208 | 0.601 | 0.353 | 4 | OAT |
| MUC5B | 0.603 | 0.973815733 | 0.206 | 0.244 | 0.036 | 4 | MUC5B |
| RRBP1 | 0.603 | 0.300022083 | 0.206 | 0.446 | 0.214 | 4 | RRBP1 |
| EPHX1 | 0.602 | 0.276279911 | 0.204 | 0.506 | 0.271 | 4 | EPHX1 |
| MAGED2 | 0.601 | 0.311387152 | 0.202 | 0.4 | 0.177 | 4 | MAGED2 |
| PRDX1 | 0.601 | 0.280189614 | 0.202 | 0.86 | 0.701 | 4 | PRDX1 |
| Glandular | | | | | | | |
| S100A6 | 0.8 | 1.35280518 | 0.6 | 0.852 | 0.592 | 13 | S100A6 |
| ANXA1 | 0.784 | 1.088044882 | 0.568 | 0.872 | 0.609 | 13 | ANXA1 |
| AGR2 | 0.774 | 2.294444177 | 0.548 | 0.743 | 0.423 | 13 | AGR2 |
| KRT19 | 0.749 | 1.253447957 | 0.498 | 0.76 | 0.477 | 13 | KRT19 |
| TMSB10 | 0.698 | 0.649579784 | 0.396 | 0.814 | 0.74 | 13 | TMSB10 |
| LGALS3 | 0.691 | 0.859573352 | 0.382 | 0.721 | 0.586 | 13 | LGALS3 |
| MTRNR2L1 | 0.686 | 1.051963806 | 0.372 | 0.834 | 0.687 | 13 | MTRNR2L1 |
| PRSS23 | 0.684 | 1.091534416 | 0.368 | 0.548 | 0.261 | 13 | PRSS23 |
| MT-RNR1 | 0.684 | 0.589689276 | 0.368 | 0.946 | 0.881 | 13 | MT-RNR1 |
| PERP | 0.68 | 0.787557181 | 0.36 | 0.679 | 0.511 | 13 | PERP |
| TFF3 | 0.672 | 2.994260234 | 0.344 | 0.447 | 0.157 | 13 | TFF3 |
| GSTP1 | 0.669 | 0.906809606 | 0.338 | 0.61 | 0.421 | 13 | GSTP1 |
| MTRNR2L8 | 0.669 | 0.816990646 | 0.338 | 0.84 | 0.704 | 13 | MTRNR2L8 |
| DUSP1 | 0.666 | 0.695379045 | 0.332 | 0.752 | 0.656 | 13 | DUSP1 |
| CP | 0.663 | 1.146629076 | 0.326 | 0.499 | 0.239 | 13 | CP |
| MTRNR2L2 | 0.656 | 0.833576714 | 0.312 | 0.757 | 0.586 | 13 | MTRNR2L2 |
| MTRNR2L12 | 0.654 | 0.767507171 | 0.308 | 0.804 | 0.662 | 13 | MTRNR2L12 |
| MUC5B | 0.651 | 3.007009151 | 0.302 | 0.359 | 0.08 | 13 | MUC5B |
| EGR1 | 0.643 | 0.696758057 | 0.286 | 0.654 | 0.512 | 13 | EGR1 |
| ACTG1 | 0.641 | 0.628712245 | 0.282 | 0.668 | 0.584 | 13 | ACTG1 |
| TSPAN1 | 0.64 | 1.155334165 | 0.28 | 0.37 | 0.117 | 13 | TSPAN1 |
| UBC | 0.638 | 0.537873167 | 0.276 | 0.727 | 0.661 | 13 | UBC |
| EZR | 0.633 | 0.690593899 | 0.266 | 0.606 | 0.457 | 13 | EZR |
| ANXA2 | 0.632 | 0.672064728 | 0.264 | 0.596 | 0.489 | 13 | ANXA2 |
| ZFAS1 | 0.631 | 0.767632684 | 0.262 | 0.52 | 0.339 | 13 | ZFAS1 |
| ELF3 | 0.629 | 0.658718093 | 0.258 | 0.593 | 0.451 | 13 | ELF3 |
| SERPINB3 | 0.628 | 1.5263778 | 0.256 | 0.397 | 0.187 | 13 | SERPINB3 |
| MSMB | 0.627 | 3.007435162 | 0.254 | 0.575 | 0.502 | 13 | MSMB |
| MT-CO3 | 0.627 | 0.410873136 | 0.254 | 0.846 | 0.775 | 13 | MT-CO3 |
| AQP3 | 0.626 | 0.945237909 | 0.252 | 0.53 | 0.397 | 13 | AQP3 |
| HES1 | 0.625 | 0.811851733 | 0.25 | 0.509 | 0.331 | 13 | HES1 |
| RPL28 | 0.623 | 0.557920229 | 0.246 | 0.61 | 0.504 | 13 | RPL28 |
| MT-CO2 | 0.623 | 0.529492878 | 0.246 | 0.748 | 0.656 | 13 | MT-CO2 |
| ALDH1A1 | 0.622 | 0.616185431 | 0.244 | 0.582 | 0.467 | 13 | ALDH1A1 |
| TACSTD2 | 0.62 | 0.635254468 | 0.24 | 0.619 | 0.546 | 13 | TACSTD2 |
| AHNAK | 0.619 | 0.820510288 | 0.238 | 0.372 | 0.154 | 13 | AHNAK |
| PTMA | 0.618 | 0.552450569 | 0.236 | 0.625 | 0.535 | 13 | PTMA |
| MTRNR2L3 | 0.611 | 0.805684224 | 0.222 | 0.519 | 0.357 | 13 | MTRNR2L3 |
| KLF4 | 0.611 | 0.800810628 | 0.222 | 0.371 | 0.177 | 13 | KLF4 |
| S100P | 0.61 | 1.274926404 | 0.22 | 0.269 | 0.06 | 13 | S100P |
| GOLM1 | 0.609 | 1.09889442 | 0.218 | 0.255 | 0.045 | 13 | GOLM1 |
| MT-ND2 | 0.609 | 0.389584255 | 0.218 | 0.818 | 0.766 | 13 | MT-ND2 |
| BPIFB2 | 0.608 | 2.509828044 | 0.216 | 0.273 | 0.074 | 13 | BPIFB2 |
| ALCAM | 0.607 | 0.72232311 | 0.214 | 0.4 | 0.224 | 13 | ALCAM |
| CLU | 0.605 | 0.899474593 | 0.21 | 0.59 | 0.521 | 13 | CLU |
| F3 | 0.605 | 0.684257246 | 0.21 | 0.47 | 0.318 | 13 | F3 |
| MT-ND4 | 0.605 | 0.450993624 | 0.21 | 0.783 | 0.718 | 13 | MT-ND4 |
| MYL12B | 0.603 | 0.611252682 | 0.206 | 0.527 | 0.427 | 13 | MYL12B |
| VMO1 | 0.602 | 1.062183781 | 0.204 | 0.324 | 0.142 | 13 | VMO1 |
| SCGB3A1 | 0.602 | 0.976992844 | 0.204 | 0.549 | 0.438 | 13 | SCGB3A1 |
| LCN2 | 0.602 | 0.9564554 | 0.204 | 0.462 | 0.319 | 13 | LCN2 |
| C19orf33 | 0.601 | 0.785068347 | 0.202 | 0.248 | 0.05 | 13 | C19orf33 |
| LYZ1 | 0.919 | 1.59011355 | 0.838 | 0.999 | 0.908 | 3 | LYZ |
| C6orf581 | 0.861 | 1.79730313 | 0.722 | 0.907 | 0.474 | 3 | C6orf58 |
| ZG16B1 | 0.857 | 1.570484599 | 0.714 | 0.982 | 0.775 | 3 | ZG16B |
| STATH1 | 0.853 | 1.365532641 | 0.706 | 0.987 | 0.862 | 3 | STATH |
| BPIFB11 | 0.841 | 1.029133475 | 0.682 | 0.993 | 0.917 | 3 | BPIFB1 |
| BPIFA11 | 0.823 | 1.359879828 | 0.646 | 0.986 | 0.897 | 3 | BPIFA1 |
| LTF1 | 0.821 | 0.919819961 | 0.642 | 0.997 | 0.719 | 3 | LTF |
| ODAM1 | 0.79 | 1.011042395 | 0.58 | 0.833 | 0.319 | 3 | ODAM |
| RP11-1143G9.41 | 0.782 | 1.749606711 | 0.564 | 0.873 | 0.486 | 3 | RP11-1143G9.4 |
| PIP1 | 0.746 | 1.163457762 | 0.492 | 0.868 | 0.568 | 3 | PIP |
| DMBT11 | 0.72 | 0.732479296 | 0.44 | 0.887 | 0.505 | 3 | DMBT1 |
| PHLDA11 | 0.685 | 0.749770961 | 0.37 | 0.597 | 0.241 | 3 | PHLDA1 |
| RNASE11 | 0.683 | 0.590324665 | 0.366 | 0.696 | 0.345 | 3 | RNASE1 |
| TCN11 | 0.679 | 0.429271696 | 0.358 | 0.956 | 0.728 | 3 | TCN1 |
| AZGP11 | 0.675 | 0.274396787 | 0.35 | 0.984 | 0.766 | 3 | AZGP1 |
| CA21 | 0.672 | 0.732913553 | 0.344 | 0.526 | 0.192 | 3 | CA2 |
| HP1 | 0.67 | 1.177238459 | 0.34 | 0.421 | 0.081 | 3 | HP |
| NUCB21 | 0.664 | 0.494300799 | 0.328 | 0.752 | 0.48 | 3 | NUCB2 |
| PIGR1 | 0.661 | 0.30092324 | 0.322 | 0.995 | 0.93 | 3 | PIGR |
| XBP11 | 0.657 | 0.394860843 | 0.314 | 0.908 | 0.706 | 3 | XBP1 |
| S100A11 | 0.641 | 0.596301704 | 0.282 | 0.397 | 0.112 | 3 | S100A1 |
| LPO1 | 0.631 | 0.767577263 | 0.262 | 0.309 | 0.048 | 3 | LPO |
| SLC31A21 | 0.631 | 0.653643925 | 0.262 | 0.318 | 0.055 | 3 | SLC31A2 |
| SSR41 | 0.631 | 0.322696022 | 0.262 | 0.827 | 0.622 | 3 | SSR4 |
| MGLL1 | 0.627 | 0.486591366 | 0.254 | 0.499 | 0.247 | 3 | MGLL |
| PHB1 | 0.604 | 0.425037011 | 0.208 | 0.399 | 0.192 | 3 | PHB |
| PPP1R1B1 | 0.603 | 0.318268587 | 0.206 | 0.425 | 0.21 | 3 | PPP1R1B |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 14. The clusters are as shown in FIG. 2E. The biomarkers listed in Table 14 may be used to provide differential expression across disease state (non-poly vs. polyp), with biomarkers in non-polyp cells representing function to be restored in polyp cells for proper anti-microbial function.

TABLE 14

| | p_val | avg_diff | pct. 1 | pct. 2 |
|---|---|---|---|---|
| STATH | 0 | −2.71508123 | 0.052 | 0.861 |
| MSMB | 1.59E−139 | −1.850522775 | 0.177 | 0.514 |
| SCGB1A1 | 2.32E−95 | −1.466947936 | 0.182 | 0.467 |
| SCGB3A1 | 2.42E−105 | −1.196705282 | 0.008 | 0.203 |
| ALDH3A1 | 2.13E−167 | −1.178993007 | 0.213 | 0.608 |
| ZG16B | 4.33E−201 | −1.145932797 | 0.032 | 0.399 |
| EPAS1 | 7.23E−246 | −1.049858044 | 0.404 | 0.837 |
| PSCA | 3.33E−81 | −1.010578011 | 0.124 | 0.377 |
| LYPD2 | 2.03E−55 | −0.877314648 | 0.161 | 0.37 |
| LYZ | 1.57E−138 | −0.840616683 | 0.168 | 0.533 |
| STEAP4 | 2.28E−80 | −0.818289927 | 0.048 | 0.254 |
| S100A9 | 9.10E−41 | −0.745777706 | 0.084 | 0.237 |
| CLCA4 | 1.48E−62 | −0.73962304 | 0.021 | 0.166 |
| GSTA1 | 1.14E−81 | −0.728891829 | 0.009 | 0.17 |
| TMEM213 | 1.64E−73 | −0.713439429 | 0.115 | 0.353 |
| AQP3 | 6.09E−132 | −0.695101724 | 0.61 | 0.881 |
| FMO3 | 5.59E−66 | −0.684049005 | 0.182 | 0.423 |
| CLU | 1.45E−112 | −0.682341243 | 0.133 | 0.437 |
| CYP4B1 | 1.27E−82 | −0.681437204 | 0.205 | 0.489 |
| KIF21A | 1.29E−69 | −0.61271861 | 0.113 | 0.342 |
| CSRP2 | 9.68E−66 | −0.611879016 | 0.043 | 0.221 |
| S100A4 | 8.21E−50 | −0.610773803 | 0.389 | 0.598 |
| UGT2A2 | 7.03E−92 | −0.610296158 | 0.401 | 0.705 |
| GLUL | 6.62E−93 | −0.607823447 | 0.453 | 0.744 |
| XIST | 1.68E−67 | −0.597632763 | 0.186 | 0.435 |
| CD74 | 2.18E−41 | −0.584196704 | 0.293 | 0.479 |
| PTGS2 | 4.44E−38 | −0.580380618 | 0.046 | 0.171 |
| HPGD | 1.99E−47 | −0.576662755 | 0.185 | 0.389 |
| AQP5 | 5.42E−70 | −0.568264304 | 0.21 | 0.472 |
| FAM3D | 6.39E−61 | −0.558455396 | 0.13 | 0.347 |
| MMP10 | 1.32E−27 | −0.556769111 | 0.15 | 0.295 |
| HES1 | 3.55E−65 | −0.554127493 | 0.465 | 0.708 |
| EGFR | 9.85E−53 | −0.55310034 | 0.113 | 0.307 |
| AKR1C3 | 1.88E−57 | −0.542899323 | 0.177 | 0.403 |
| CYP2A13 | 1.00E−36 | −0.539468109 | 0.056 | 0.185 |
| IFI27 | 2.36E−35 | −0.539290257 | 0.302 | 0.477 |
| PTPN13 | 1.50E−60 | −0.538957259 | 0.1 | 0.304 |
| ALDH2 | 1.86E−68 | −0.536821088 | 0.163 | 0.407 |
| ALDH3A2 | 3.83E−53 | −0.486462202 | 0.214 | 0.44 |
| STAT1 | 1.98E−44 | −0.485796462 | 0.063 | 0.213 |
| GDF15 | 3.17E−29 | −0.482241927 | 0.187 | 0.345 |
| IL33 | 4.20E−58 | −0.472573585 | 0.163 | 0.383 |
| MKL2 | 5.02E−46 | −0.468504065 | 0.095 | 0.264 |
| SLC15A2 | 4.56E−46 | −0.466526638 | 0.082 | 0.246 |
| HLA-DRA | 2.35E−31 | −0.466264773 | 0.16 | 0.318 |
| AKR1C2 | 3.11E−44 | −0.464332133 | 0.12 | 0.297 |
| PRKAR2B | 4.94E−56 | −0.459584279 | 0.013 | 0.135 |
| TSC22D1 | 5.62E−56 | −0.457239076 | 0.378 | 0.623 |
| HLA-DRB5 | 7.85E−54 | −0.455475057 | 0.014 | 0.135 |
| PTN | 1.95E−42 | −0.45365115 | 0.101 | 0.264 |
| SEMA3A | 5.26E−44 | −0.449672282 | 0.026 | 0.145 |
| CA12 | 2.15E−53 | −0.446576943 | 0.096 | 0.274 |
| DDIT4 | 1.18E−50 | −0.44642776 | 0.232 | 0.453 |
| HLA-B | 1.20E−62 | −0.443646224 | 0.762 | 0.882 |
| ALDH1A1 | 1.69E−61 | −0.442799839 | 0.527 | 0.761 |
| SNHG8 | 3.65E−42 | −0.440096004 | 0.306 | 0.516 |
| CFD | 6.17E−17 | −0.431431158 | 0.094 | 0.184 |
| ANK3 | 2.35E−42 | −0.429296543 | 0.121 | 0.291 |
| IER3 | 9.76E−57 | −0.427752161 | 0.486 | 0.723 |
| OGFRL1 | 9.90E−40 | −0.421110687 | 0.087 | 0.239 |
| LTF | 6.01E−69 | −0.415389887 | 0.034 | 0.194 |
| MUC5B | 1.30E−23 | −0.410620145 | 0.043 | 0.129 |
| C6orf58 | 1.59E−59 | −0.402795411 | 0.001 | 0.102 |
| IL18 | 1.01E−41 | −0.397366042 | 0.037 | 0.158 |
| APOD | 3.32E−33 | −0.397287002 | 0.015 | 0.101 |
| ADIRF | 8.02E−56 | −0.392498908 | 0.244 | 0.474 |
| RNF152 | 4.83E−36 | −0.391970357 | 0.076 | 0.214 |
| F3 | 1.24E−63 | −0.390590262 | 0.581 | 0.814 |
| RIMS1 | 1.02E−30 | −0.389698053 | 0.136 | 0.284 |
| PRDX6 | 3.60E−52 | −0.389065586 | 0.249 | 0.474 |
| TXNIP | 9.62E−39 | −0.38799494 | 0.466 | 0.663 |
| ZFP36L1 | 1.49E−59 | −0.385783095 | 0.576 | 0.8 |
| NUCB2 | 3.46E−30 | −0.383319076 | 0.16 | 0.313 |
| PAX7 | 1.75E−33 | −0.382975469 | 0.02 | 0.113 |
| CFH | 1.88E−42 | −0.382488997 | 0.191 | 0.382 |
| CD82 | 2.26E−49 | −0.380486231 | 0.192 | 0.396 |
| CHL1 | 1.88E−50 | −0.379339502 | 0.183 | 0.386 |
| FAM107A | 3.29E−44 | −0.37332943 | 0.169 | 0.354 |
| SEMA3C | 4.55E−27 | −0.371811718 | 0.084 | 0.203 |
| SCNN1B | 7.26E−28 | −0.37107156 | 0.041 | 0.14 |
| SAMHD1 | 7.19E−40 | −0.369902378 | 0.067 | 0.204 |
| RARRES3 | 8.21E−34 | −0.368952679 | 0.109 | 0.254 |
| CYP2J2 | 1.79E−30 | −0.367502192 | 0.092 | 0.221 |
| ITGA2 | 1.09E−21 | −0.366961163 | 0.085 | 0.189 |
| ID1 | 3.64E−41 | −0.366070027 | 0.54 | 0.728 |
| FLRT3 | 1.21E−26 | −0.3647232 | 0.023 | 0.105 |
| TSPAN8 | 7.17E−29 | −0.364011624 | 0.08 | 0.199 |
| PTGFR | 1.06E−39 | −0.362091317 | 0.014 | 0.109 |
| HLA-A | 5.53E−34 | −0.361795651 | 0.545 | 0.714 |
| TRIM24 | 1.53E−26 | −0.360387232 | 0.085 | 0.204 |
| NEBL | 8.93E−26 | −0.357302114 | 0.062 | 0.168 |
| EFCAB4A | 5.17E−32 | −0.351945326 | 0.079 | 0.207 |
| LRIG3 | 4.45E−35 | −0.351790903 | 0.069 | 0.196 |
| GK5 | 1.84E−37 | −0.351340077 | 0.05 | 0.17 |
| CYP2F1 | 5.66E−16 | −0.349150715 | 0.073 | 0.155 |
| DHCR24 | 1.15E−39 | −0.349125422 | 0.209 | 0.396 |
| ACAP2 | 5.26E−37 | −0.346281353 | 0.163 | 0.33 |
| ALCAM | 3.80E−40 | −0.34547376 | 0.449 | 0.652 |
| SPINK5 | 9.38E−18 | −0.344528347 | 0.038 | 0.111 |
| METTL7A | 1.64E−43 | −0.343608844 | 0.198 | 0.389 |
| NFIB | 3.52E−30 | −0.342032947 | 0.14 | 0.285 |
| PIGR | 2.47E−29 | −0.341117726 | 0.486 | 0.662 |
| EGR1 | 1.37E−42 | −0.33979055 | 0.682 | 0.846 |
| RTN4 | 1.34E−33 | −0.3395299 | 0.194 | 0.36 |
| HLA-DRB1 | 1.11E−25 | −0.339399041 | 0.098 | 0.217 |
| RNA18S5 | 1.06E−12 | −0.338925119 | 0.121 | 0.206 |
| TMX4 | 1.97E−25 | −0.337007532 | 0.097 | 0.217 |
| FAM3B | 2.19E−32 | −0.334800464 | 0.134 | 0.282 |
| DUSP6 | 6.21E−30 | −0.331906261 | 0.127 | 0.265 |
| NET1 | 1.92E−35 | −0.330921721 | 0.281 | 0.47 |
| PIK3R3 | 5.53E−28 | −0.330250198 | 0.138 | 0.277 |
| FMO2 | 6.12E−26 | −0.32980621 | 0.23 | 0.383 |
| PARP14 | 5.21E−24 | −0.329491245 | 0.063 | 0.164 |
| PRSS23 | 4.51E−53 | −0.329333712 | 0.701 | 0.878 |
| ITM2B | 9.47E−52 | −0.327416798 | 0.638 | 0.836 |
| ARSD | 4.14E−33 | −0.324251056 | 0.062 | 0.18 |
| PRDX1 | 2.44E−36 | −0.323532906 | 0.661 | 0.804 |
| TOB1 | 2.88E−42 | −0.323069722 | 0.413 | 0.626 |
| GOLGB1 | 3.34E−33 | −0.32265282 | 0.297 | 0.48 |
| DUSP1 | 1.40E−42 | −0.322601256 | 0.796 | 0.902 |
| CLEC2B | 8.72E−33 | −0.321594354 | 0.173 | 0.33 |
| COBLL1 | 5.69E−26 | −0.319632794 | 0.041 | 0.133 |
| SDC4 | 3.32E−37 | −0.319488124 | 0.253 | 0.438 |
| AHR | 5.77E−27 | −0.317389788 | 0.183 | 0.33 |
| KIAA1109 | 4.24E−18 | −0.315078036 | 0.068 | 0.156 |
| HLA-E | 3.44E−32 | −0.313694012 | 0.467 | 0.645 |
| CRNDE | 5.54E−28 | −0.313362679 | 0.021 | 0.104 |
| MDFIC | 1.81E−25 | −0.312929662 | 0.036 | 0.125 |
| PRKCD | 1.03E−23 | −0.31162031 | 0.033 | 0.115 |
| SKIL | 1.24E−32 | −0.311004205 | 0.117 | 0.256 |
| LGALS8 | 3.69E−32 | −0.310774431 | 0.151 | 0.301 |
| DST | 3.06E−14 | −0.310199265 | 0.134 | 0.227 |
| ZFP36L2 | 2.91E−33 | −0.309971294 | 0.219 | 0.389 |
| ADD3 | 2.81E−37 | −0.308893519 | 0.133 | 0.286 |
| CTSB | 1.86E−37 | −0.308259212 | 0.601 | 0.776 |
| TMPRSS11D | 3.82E−24 | −0.306367903 | 0.071 | 0.175 |
| DAPL1 | 1.76E−26 | −0.305715651 | 0.057 | 0.157 |
| BTG1 | 8.46E−45 | −0.304774055 | 0.305 | 0.514 |
| FAM129A | 3.70E−26 | −0.304015962 | 0.135 | 0.266 |
| CTD-2135D7.5 | 5.23E−31 | −0.302251823 | 0.026 | 0.115 |
| AVPI1 | 1.49E−26 | −0.298721722 | 0.1 | 0.219 |
| ABCA13 | 1.88E−23 | −0.298653995 | 0.168 | 0.3 |
| PLCB4 | 1.64E−16 | −0.297627682 | 0.092 | 0.185 |
| CDC42BPA | 3.96E−19 | −0.29725952 | 0.075 | 0.168 |
| RP11-485G4.2 | 4.90E−23 | −0.297179182 | 0.045 | 0.133 |
| RHOBTB3 | 1.16E−19 | −0.294515781 | 0.03 | 0.101 |
| NFIA | 1.46E−43 | −0.294048553 | 0.112 | 0.266 |
| SCNN1A | 2.23E−33 | −0.293620699 | 0.167 | 0.322 |
| HNMT | 8.30E−37 | −0.291838598 | 0.068 | 0.191 |
| MUC15 | 3.08E−25 | −0.289014556 | 0.027 | 0.108 |
| TRAK2 | 6.85E−20 | −0.288413264 | 0.069 | 0.162 |
| CHP2 | 3.95E−21 | −0.288214477 | 0.156 | 0.279 |
| B4GALT5 | 3.36E−24 | −0.285429938 | 0.09 | 0.201 |
| KIAA1324 | 8.11E−30 | −0.285264429 | 0.087 | 0.207 |
| TFDP2 | 1.83E−28 | −0.284224084 | 0.082 | 0.197 |
| SNRNP200 | 7.27E−24 | −0.284131955 | 0.08 | 0.185 |
| EXPH5 | 1.98E−23 | −0.28364693 | 0.14 | 0.263 |
| EPB41L4A-AS1 | 1.71E−23 | −0.283544897 | 0.092 | 0.201 |
| PIK3R1 | 2.29E−21 | −0.282955099 | 0.144 | 0.262 |
| CRYM | 7.17E−25 | −0.280250824 | 0.059 | 0.154 |
| MFSD1 | 4.15E−19 | −0.279932561 | 0.127 | 0.237 |
| EGR2 | 1.78E−24 | −0.279173514 | 0.162 | 0.293 |
| THOC2 | 1.14E−17 | −0.278443264 | 0.093 | 0.188 |
| PDLIM1 | 5.89E−35 | −0.277698497 | 0.193 | 0.355 |
| CTSL | 2.71E−17 | −0.277665337 | 0.056 | 0.137 |
| SLC44A2 | 9.60E−19 | −0.277472697 | 0.067 | 0.156 |
| RPL30 | 2.72E−38 | −0.27665431 | 0.893 | 0.949 |
| SLITRK6 | 1.18E−22 | −0.274812365 | 0.046 | 0.132 |
| MPP7 | 3.75E−20 | −0.274584078 | 0.041 | 0.121 |
| LSAMP | 7.85E−21 | −0.274541983 | 0.035 | 0.11 |
| MX1 | 1.69E−22 | −0.274469055 | 0.055 | 0.146 |
| HLA-F | 5.52E−24 | −0.27351162 | 0.128 | 0.249 |
| PERP | 2.85E−34 | −0.272842871 | 0.708 | 0.855 |
| ABCC4 | 1.32E−29 | −0.272623077 | 0.023 | 0.104 |
| AHNAK | 1.05E−42 | −0.272513926 | 0.354 | 0.561 |
| JUNB | 7.45E−37 | −0.272332004 | 0.721 | 0.866 |
| CLSTN1 | 3.79E−25 | −0.272221321 | 0.122 | 0.242 |
| SREBF1 | 4.99E−23 | −0.269017784 | 0.03 | 0.107 |
| ANPEP | 9.66E−23 | −0.268907571 | 0.06 | 0.152 |
| PI3 | 1.03E−25 | −0.267656238 | 0.121 | 0.243 |
| BAZ2B | 5.82E−21 | −0.266825328 | 0.16 | 0.282 |
| SEPP1 | 5.70E−37 | −0.265426795 | 0.257 | 0.437 |
| APLP2 | 3.02E−47 | −0.265402627 | 0.434 | 0.652 |
| ID2 | 1.80E−32 | −0.26429674 | 0.259 | 0.429 |
| NUPR1 | 7.30E−24 | −0.263926284 | 0.248 | 0.393 |
| NR4A2 | 5.13E−20 | −0.263739929 | 0.155 | 0.272 |
| XAF1 | 1.25E−19 | −0.26324096 | 0.032 | 0.102 |
| DUSP2 | 4.52E−24 | −0.262115278 | 0.148 | 0.27 |
| BAG1 | 6.64E−28 | −0.262030232 | 0.203 | 0.351 |
| OXCT1 | 7.00E−27 | −0.261169291 | 0.04 | 0.129 |
| RBM25 | 1.28E−31 | −0.26085835 | 0.185 | 0.337 |
| RP11-304L19.5 | 1.38E−24 | −0.259771546 | 0.084 | 0.189 |
| EPHX1 | 1.42E−34 | −0.258463782 | 0.238 | 0.404 |
| ATRX | 7.49E−25 | −0.257739509 | 0.189 | 0.325 |
| SCARB2 | 1.15E−20 | −0.257706655 | 0.128 | 0.24 |
| CXCL1 | 8.49E−07 | −0.256523924 | 0.169 | 0.235 |
| ODF2L | 1.24E−13 | −0.256016657 | 0.067 | 0.142 |
| ERN2 | 2.53E−17 | −0.255657105 | 0.126 | 0.229 |
| SORD | 1.35E−23 | −0.25248636 | 0.183 | 0.315 |
| PKP2 | 1.47E−23 | −0.251220523 | 0.062 | 0.156 |
| S100A14 | 3.86E−38 | −0.250861844 | 0.192 | 0.354 |
| ZNF90 | 3.85E−10 | 0.275033064 | 0.192 | 0.134 |
| MT-ND4 | 1.77E−35 | 0.276955915 | 0.623 | 0.677 |
| BPIFB1 | 2.37E−62 | 0.308538704 | 0.463 | 0.641 |
| TMSB4X | 3.30E−28 | 0.31206408 | 0.71 | 0.654 |
| SEC14L1 | 1.81E−13 | 0.323868255 | 0.171 | 0.121 |
| SERINC5 | 4.63E−16 | 0.331450337 | 0.114 | 0.061 |
| SERPINB13 | 1.33E−10 | 0.339690582 | 0.137 | 0.084 |
| ISG20 | 4.28E−18 | 0.341789927 | 0.164 | 0.113 |
| RPS7 | 3.16E−37 | 0.345606744 | 0.654 | 0.588 |
| STOM | 2.49E−15 | 0.367717307 | 0.173 | 0.111 |
| PFKP | 3.31E−17 | 0.37025889 | 0.121 | 0.068 |
| GCNT3 | 7.70E−21 | 0.38333524 | 0.105 | 0.036 |
| KRT23 | 1.97E−19 | 0.383573206 | 0.3 | 0.237 |
| RPL36 | 2.41E−50 | 0.392935803 | 0.718 | 0.635 |
| MTRNR2L9 | 1.10E−13 | 0.393507188 | 0.184 | 0.128 |
| RPL13A | 4.04E−84 | 0.403306885 | 0.925 | 0.855 |
| MRPS6 | 6.04E−19 | 0.403556308 | 0.174 | 0.111 |
| RPL7 | 1.47E−31 | 0.410233146 | 0.22 | 0.168 |
| SSR4 | 2.13E−57 | 0.419596161 | 0.551 | 0.501 |
| FBXO32 | 8.75E−21 | 0.428927165 | 0.147 | 0.072 |
| NOS2 | 3.19E−25 | 0.431309976 | 0.298 | 0.219 |
| RPSAP58 | 4.90E−37 | 0.445291779 | 0.288 | 0.216 |
| CTD-2228K2.5 | 1.48E−21 | 0.445823055 | 0.17 | 0.102 |
| LBH | 2.87E−24 | 0.455833528 | 0.247 | 0.17 |
| PLS1 | 5.75E−31 | 0.45600413 | 0.228 | 0.153 |
| RPL10 | 2.18E−52 | 0.468364878 | 0.666 | 0.554 |
| MT-ND5 | 1.93E−37 | 0.472072742 | 0.488 | 0.433 |
| COL6A1 | 2.02E−22 | 0.474572444 | 0.122 | 0.052 |
| RPL21 | 3.80E−41 | 0.47479087 | 0.331 | 0.254 |
| S100A10 | 9.30E−39 | 0.490045104 | 0.438 | 0.349 |
| SLC6A8 | 2.20E−32 | 0.495334962 | 0.144 | 0.042 |
| MTRNR2L13 | 1.70E−25 | 0.499400304 | 0.28 | 0.182 |
| MTRNR2L10 | 1.19E−26 | 0.500350954 | 0.173 | 0.09 |
| MZB1 | 1.45E−44 | 0.504175264 | 0.105 | 0.011 |
| UBBP4 | 5.11E−42 | 0.505949149 | 0.305 | 0.205 |
| NTRK2 | 6.03E−36 | 0.514102693 | 0.131 | 0.033 |
| CEACAM5 | 1.32E−19 | 0.519820237 | 0.129 | 0.077 |
| SRGN | 1.53E−28 | 0.52524372 | 0.133 | 0.044 |
| RPS27 | 1.24E−57 | 0.549278517 | 0.644 | 0.501 |
| MTRNR2L1 | 1.09E−152 | 0.55292246 | 0.945 | 0.787 |
| KRT6A | 3.73E−21 | 0.558241405 | 0.17 | 0.094 |
| LDHA | 2.76E−53 | 0.563766512 | 0.401 | 0.297 |
| MT-CO2 | 1.17E−65 | 0.572124572 | 0.714 | 0.65 |
| CTD-2319I12.1 | 2.85E−33 | 0.577515385 | 0.42 | 0.341 |
| MTRNR2L3 | 1.43E−55 | 0.58878424 | 0.607 | 0.473 |
| LGALS7 | 3.09E−33 | 0.606327244 | 0.154 | 0.064 |
| MTRNR2L2 | 1.61E−57 | 0.61354727 | 0.797 | 0.683 |
| GSN | 2.15E−66 | 0.624603733 | 0.505 | 0.409 |
| TNC | 2.93E−38 | 0.629799385 | 0.268 | 0.178 |
| NDRG1 | 1.56E−43 | 0.633038562 | 0.21 | 0.082 |
| CD44 | 2.65E−61 | 0.651036784 | 0.407 | 0.292 |
| SERPINB3 | 1.04E−162 | 0.670871272 | 0.948 | 0.858 |
| SLC6A14 | 1.86E−60 | 0.680892566 | 0.444 | 0.308 |
| RPL18A | 2.84E−93 | 0.684841736 | 0.548 | 0.394 |
| S100A2 | 8.69E−42 | 0.68794328 | 0.553 | 0.445 |
| RPS4Y1 | 1.01E−75 | 0.705613041 | 0.115 | 0.002 |
| SERPINB4 | 3.60E−97 | 0.732102927 | 0.695 | 0.514 |
| IGFBP5 | 5.46E−48 | 0.752036004 | 0.302 | 0.177 |
| IGHM | 4.78E−53 | 0.782538682 | 0.139 | 0.02 |
| HS3ST1 | 2.33E−113 | 0.826294931 | 0.54 | 0.352 |
| IGHG3 | 1.60E−68 | 0.848699988 | 0.24 | 0.082 |
| CCL26 | 1.07E−52 | 0.864768495 | 0.135 | 0.021 |
| MIF | 2.88E−129 | 0.898184557 | 0.44 | 0.239 |
| IGHG1 | 4.26E−73 | 0.969566415 | 0.218 | 0.052 |
| CDH26 | 4.44E−143 | 1.150469637 | 0.358 | 0.072 |
| ALOX15 | 2.54215055214217e−318 | 1.159513656 | 0.806 | 0.583 |
| SERPINB2 | 1.37E−88 | 1.246654303 | 0.215 | 0.035 |
| IGHA2 | 1.15E−122 | 1.295455743 | 0.303 | 0.058 |
| EGLN3 | 1.96E−186 | 1.40367355 | 0.341 | 0.037 |
| TFF3 | 2.55E−96 | 1.464232926 | 0.555 | 0.289 |
| IGFBP3 | 3.78E−212 | 1.532240634 | 0.579 | 0.223 |
| CST2 | 1.36E−189 | 1.711634479 | 0.339 | 0.027 |
| IGHA1 | 1.32E−302 | 1.712091312 | 0.676 | 0.327 |
| IGJ | 0 | 1.874116686 | 0.645 | 0.164 |
| PTHLH | 4.27533283775327e−318 | 1.982875419 | 0.486 | 0.031 |
| IGHG4 | 6.75E−202 | 1.98952236 | 0.337 | 0.034 |
| CST1 | 0 | 2.049784182 | 0.747 | 0.14 |
| POSTN | 1.26E−279 | 2.167255965 | 0.583 | 0.138 |
| CST4 | 1.72E−300 | 2.55451304 | 0.512 | 0.06 |

In certain example embodiments, the biomarkers for detection of cell phenotypes and/or targets for modulating fibroblast cell proliferation, differentiation, maintenance, and/or function in barrier tissues comprising modulating one or more genes or gene expression products in Table 14. The clusters are as shown in FIG. 2A. The biomarkers listed in Table 15 highlight changes in basal cell compartment showing differential expression in differentiating/secretory cells in polyp vs non-polyp

TABLE 15

| | p_val | avg_diff | pct. 1 | pct. 2 |
|---|---|---|---|---|
| POSTN | 0 | 2.238280216 | 0.942 | 0.455 |
| IGJ | 4.36E−153 | 1.79402201 | 0.525 | 0.193 |
| PTHLH | 8.77E−132 | 1.712775245 | 0.466 | 0.097 |
| IGHG4 | 1.64E−75 | 1.583915029 | 0.236 | 0.036 |
| IGHA1 | 4.54E−143 | 1.512786137 | 0.564 | 0.346 |
| ALOX15 | 6.26E−259 | 1.441849042 | 0.791 | 0.545 |
| SERPINB2 | 6.26E−59 | 1.3718054 | 0.214 | 0.019 |
| CCL26 | 1.03E−53 | 1.341471405 | 0.183 | 0.027 |
| RPS4Y1 | 2.22E−68 | 1.081279884 | 0.182 | 0.004 |
| HS3ST1 | 1.61E−86 | 1.049410017 | 0.381 | 0.237 |
| SERPINB10 | 1.52E−57 | 1.017031552 | 0.197 | 0.016 |
| CDH26 | 1.57E−53 | 1.009479861 | 0.217 | 0.035 |
| MMP10 | 2.79E−77 | 0.990640738 | 0.639 | 0.453 |
| IGFBP3 | 1.13E−39 | 0.934088143 | 0.303 | 0.203 |
| IGHG1 | 1.66E−34 | 0.888879969 | 0.17 | 0.05 |
| IGHA2 | 1.12E−36 | 0.883028303 | 0.211 | 0.068 |
| NTRK2 | 7.30E−47 | 0.881532549 | 0.307 | 0.15 |
| KCNJ16 | 3.41E−52 | 0.841020641 | 0.155 | 0.004 |
| TFF3 | 6.59E−47 | 0.826044593 | 0.238 | 0.161 |
| MTRNR2L1 | 3.27E−128 | 0.811369496 | 0.91 | 0.748 |
| MTRNR2L2 | 5.33E−67 | 0.805202888 | 0.745 | 0.651 |
| MIF | 6.38E−73 | 0.794617149 | 0.389 | 0.275 |
| CD55 | 9.00E−79 | 0.776526859 | 0.515 | 0.447 |
| CTSC | 7.10E−71 | 0.757350066 | 0.405 | 0.322 |
| SLC5A3 | 4.14E−29 | 0.74796335 | 0.2 | 0.084 |
| CD44 | 2.54E−87 | 0.746206852 | 0.507 | 0.434 |
| FBXO32 | 1.70E−36 | 0.730267978 | 0.234 | 0.123 |
| FGFBP1 | 4.08E−27 | 0.726121571 | 0.143 | 0.05 |
| CST1 | 6.04E−22 | 0.696546624 | 0.125 | 0.033 |
| LGALS7 | 5.11E−31 | 0.691122339 | 0.242 | 0.15 |
| EGLN3 | 1.28E−26 | 0.664962583 | 0.156 | 0.044 |
| NOS2 | 2.24E−25 | 0.614377124 | 0.182 | 0.103 |
| CTD-2228K2.5 | 2.71E−28 | 0.610387789 | 0.175 | 0.107 |
| MRPS6 | 6.26E−38 | 0.605110982 | 0.281 | 0.199 |
| SDPR | 2.40E−14 | 0.503608536 | 0.181 | 0.126 |
| FAM110C | 1.08E−14 | 0.480041622 | 0.113 | 0.038 |
| SGK1 | 5.65E−70 | 0.45507219 | 0.551 | 0.633 |
| LOXL4 | 9.13E−16 | 0.44914342 | 0.157 | 0.105 |
| SERPINE2 | 8.00E−14 | 0.446891977 | 0.122 | 0.061 |
| CAV1 | 2.71E−12 | 0.426350759 | 0.134 | 0.076 |
| LRRC17 | 2.72E−13 | 0.425803157 | 0.106 | 0.043 |
| GPC3 | 5.35E−12 | 0.410912955 | 0.107 | 0.046 |
| SERF2 | 2.56E−85 | 0.409010923 | 0.673 | 0.726 |
| MT-CO1 | 2.03E−44 | 0.405538152 | 0.575 | 0.639 |
| LGALS3 | 1.97E−70 | 0.404739744 | 0.627 | 0.701 |
| CEBPD | 8.58E−59 | 0.386565956 | 0.44 | 0.515 |
| MT-ND5 | 2.21E−50 | 0.386461275 | 0.39 | 0.458 |
| EHF | 2.31E−60 | 0.385494053 | 0.418 | 0.48 |
| CD9 | 2.22E−72 | 0.37016221 | 0.827 | 0.883 |
| ETS2 | 1.34E−65 | 0.359948558 | 0.642 | 0.725 |
| KRT17 | 3.67E−56 | 0.356036214 | 0.509 | 0.566 |
| OAZ1 | 8.58E−59 | 0.343422608 | 0.496 | 0.555 |
| FXYD3 | 3.53E−65 | 0.334423083 | 0.737 | 0.798 |
| YBX3 | 2.17E−46 | 0.326051608 | 0.356 | 0.418 |
| LAMTOR4 | 2.66E−45 | 0.320309995 | 0.327 | 0.383 |
| UBB | 4.88E−57 | 0.310805309 | 0.759 | 0.837 |
| MTRNR2L12 | 5.18E−50 | 0.308194582 | 0.633 | 0.755 |
| MT-CO2 | 1.83E−36 | 0.306297894 | 0.567 | 0.639 |
| TSPAN3 | 5.30E−43 | 0.301703039 | 0.285 | 0.336 |
| VMP1 | 2.35E−32 | 0.296795757 | 0.286 | 0.338 |
| TCEB2 | 1.30E−44 | 0.296398857 | 0.386 | 0.442 |
| BHLHE40 | 6.75E−52 | 0.296072067 | 0.447 | 0.537 |
| TMSB10 | 2.97E−49 | 0.292202294 | 0.748 | 0.83 |
| YBX1 | 5.99E−48 | 0.280559622 | 0.457 | 0.522 |
| CXADR | 3.98E−35 | 0.278422245 | 0.204 | 0.256 |
| TMA7 | 1.09E−57 | 0.277699705 | 0.337 | 0.423 |
| MYL6 | 2.10E−61 | 0.273545605 | 0.745 | 0.835 |
| TMEM123 | 7.36E−59 | 0.270870168 | 0.572 | 0.67 |
| IGFBP7 | 3.01E−34 | 0.269606303 | 0.364 | 0.424 |
| TPI1 | 1.72E−63 | 0.269193838 | 0.424 | 0.524 |
| NPC2 | 1.07E−49 | 0.267815932 | 0.334 | 0.417 |
| LMNA | 1.36E−52 | 0.264458946 | 0.385 | 0.484 |
| NFKBIA | 7.84E−57 | 0.264281715 | 0.547 | 0.666 |
| HBEGF | 3.88E−34 | 0.263779215 | 0.247 | 0.316 |
| SOX4 | 2.55E−42 | 0.261626983 | 0.252 | 0.349 |
| TPM4 | 3.55E−47 | 0.260179931 | 0.347 | 0.435 |
| MT-ND4 | 1.08E−44 | 0.252579743 | 0.532 | 0.658 |
| CNN3 | 1.38E−42 | 0.251623148 | 0.301 | 0.376 |
| CLSTN1 | 1.14E−48 | −0.25041754 | 0.211 | 0.436 |
| WSB1 | 7.89E−53 | −0.25087787 | 0.179 | 0.402 |
| TMEM159 | 1.30E−33 | −0.251002824 | 0.077 | 0.217 |
| TPD52L1 | 9.41E−31 | −0.25150528 | 0.112 | 0.264 |
| MDFIC | 3.90E−25 | −0.25187636 | 0.037 | 0.134 |
| NFIB | 1.27E−62 | −0.252277552 | 0.235 | 0.495 |
| PSIP1 | 7.68E−31 | −0.252338959 | 0.08 | 0.215 |
| TSHZ2 | 1.03E−39 | −0.253437507 | 0.085 | 0.235 |
| TOMM7 | 1.16E−53 | −0.254490689 | 0.375 | 0.647 |
| MALAT1 | 1.90E−17 | −0.255193228 | 0.919 | 0.981 |
| ALDH3A2 | 2.01E−69 | −0.255352497 | 0.389 | 0.69 |
| FHL2 | 7.98E−45 | −0.256006645 | 0.142 | 0.332 |
| PIK3R3 | 2.31E−36 | −0.256809882 | 0.064 | 0.2 |
| SCIN | 4.27E−36 | −0.257308762 | 0.028 | 0.141 |
| SNHG7 | 2.61E−30 | −0.258519727 | 0.053 | 0.173 |
| RPL30 | 8.68E−37 | −0.258738359 | 0.92 | 0.986 |
| SAT1 | 3.42E−28 | −0.258971317 | 0.63 | 0.828 |
| DNAJB1 | 1.09E−48 | −0.259601246 | 0.411 | 0.679 |
| OXR1 | 3.63E−47 | −0.260201398 | 0.094 | 0.271 |
| RARRES1 | 7.64E−25 | −0.2607065 | 0.06 | 0.172 |
| ODF2L | 3.44E−27 | −0.261473617 | 0.056 | 0.168 |
| RPS3 | 8.91E−32 | −0.2615225 | 0.816 | 0.947 |
| ARL6IP1 | 4.40E−49 | −0.262916195 | 0.137 | 0.337 |
| TSPAN1 | 1.47E−40 | −0.263223519 | 0.12 | 0.295 |
| JAG1 | 5.08E−51 | −0.263326217 | 0.098 | 0.28 |
| PUM2 | 1.90E−32 | −0.263737617 | 0.134 | 0.299 |
| SLITRK6 | 6.24E−24 | −0.266057047 | 0.071 | 0.187 |
| MTRNR2L11 | 1.03E−09 | −0.266614803 | 0.158 | 0.23 |
| EMP2 | 6.43E−57 | −0.267075183 | 0.102 | 0.296 |
| DDIT3 | 4.66E−51 | −0.268514116 | 0.15 | 0.362 |
| NUPR1 | 2.18E−59 | −0.268961993 | 0.255 | 0.516 |
| HLA-B | 4.34E−26 | −0.269603005 | 0.715 | 0.87 |
| TXNIP | 2.77E−49 | −0.269814893 | 0.552 | 0.818 |
| HEY1 | 8.38E−40 | −0.270186832 | 0.019 | 0.13 |
| SNCG | 1.75E−34 | −0.271011015 | 0.039 | 0.161 |
| MAFB | 2.17E−30 | −0.274250782 | 0.043 | 0.155 |
| CYSTM1 | 4.77E−45 | −0.274478988 | 0.141 | 0.339 |
| ITGA2 | 1.62E−54 | −0.275873126 | 0.167 | 0.393 |
| RPL39L | 9.23E−14 | −0.276928503 | 0.076 | 0.17 |
| RBM5 | 7.45E−33 | −0.279262664 | 0.088 | 0.234 |
| MT1G | 1.53E−37 | −0.281123885 | 0.03 | 0.151 |
| ZNF750 | 1.52E−33 | −0.281347572 | 0.021 | 0.126 |
| TFCP2L1 | 6.69E−47 | −0.281699344 | 0.074 | 0.237 |
| HSPB1 | 8.66E−35 | −0.285714661 | 0.537 | 0.766 |
| PSAP | 3.64E−67 | −0.286101949 | 0.392 | 0.696 |
| PCLO | 2.48E−26 | −0.286492698 | 0.022 | 0.116 |
| WFDC2 | 1.87E−39 | −0.286631963 | 0.297 | 0.528 |
| HMGN3 | 1.15E−71 | −0.286829774 | 0.255 | 0.545 |
| CCL2 | 1.96E−06 | −0.28704629 | 0.052 | 0.106 |
| SEMA3C | 2.04E−40 | −0.289351451 | 0.074 | 0.231 |
| COBLL1 | 4.31E−32 | −0.290342907 | 0.029 | 0.139 |
| FLRT3 | 4.08E−44 | −0.290872793 | 0.092 | 0.264 |
| SMARCA2 | 7.77E−48 | −0.29347456 | 0.126 | 0.325 |
| SYNPO2 | 5.96E−24 | −0.293546343 | 0.02 | 0.108 |
| RARRES3 | 8.85E−34 | −0.295164945 | 0.055 | 0.19 |
| ARG2 | 1.02E−32 | −0.296540467 | 0.058 | 0.189 |
| ACSS3 | 3.78E−31 | −0.297250383 | 0.019 | 0.117 |
| GPX3 | 4.83E−43 | −0.298834988 | 0.03 | 0.159 |
| CLEC2B | 2.30E−43 | −0.299683122 | 0.175 | 0.387 |
| ZNF703 | 2.20E−32 | −0.299963845 | 0.031 | 0.147 |
| HES1 | 1.07E−40 | −0.300677806 | 0.442 | 0.691 |
| SPRY2 | 3.30E−31 | −0.30190828 | 0.063 | 0.196 |
| SAMHD1 | 1.04E−43 | −0.302387628 | 0.026 | 0.155 |
| CLK1 | 3.63E−68 | −0.305563584 | 0.212 | 0.485 |
| S100A9 | 9.49E−41 | −0.308734782 | 0.105 | 0.28 |
| MT2A | 1.86E−58 | −0.309333253 | 0.26 | 0.54 |
| LGALS8 | 1.95E−55 | −0.309806813 | 0.16 | 0.388 |
| PTPN13 | 1.29E−56 | −0.309889446 | 0.164 | 0.397 |
| RTN4 | 2.80E−58 | −0.312461461 | 0.18 | 0.424 |
| IFI27 | 4.12E−34 | −0.312763723 | 0.194 | 0.392 |
| SREBF1 | 3.26E−50 | −0.312788793 | 0.032 | 0.176 |
| PRKAR2B | 1.98E−42 | −0.313084442 | 0.006 | 0.107 |
| S100A14 | 1.63E−50 | −0.31423807 | 0.083 | 0.268 |
| SCGB3A1 | 3.96E−50 | −0.317897536 | 0.001 | 0.103 |
| RNA18S5 | 2.56E−15 | −0.318941111 | 0.089 | 0.196 |
| AZGP1 | 2.50E−50 | −0.320195533 | 0.004 | 0.108 |
| NEBL | 8.09E−41 | −0.321858183 | 0.024 | 0.146 |
| CCND1 | 2.59E−67 | −0.322562573 | 0.191 | 0.458 |
| CFH | 3.71E−68 | −0.32311704 | 0.221 | 0.502 |
| STAT1 | 2.99E−47 | −0.323350999 | 0.068 | 0.237 |
| PTK2 | 1.17E−32 | −0.326398074 | 0.085 | 0.238 |
| TRIM24 | 3.02E−35 | −0.33038623 | 0.067 | 0.216 |
| CXCL17 | 1.16E−50 | −0.333013814 | 0.166 | 0.395 |
| GPNMB | 4.74E−43 | −0.334679039 | 0.1 | 0.282 |
| CRNDE | 2.09E−35 | −0.334819539 | 0.033 | 0.157 |
| CTSL | 4.23E−43 | −0.337149551 | 0.066 | 0.23 |
| TMX4 | 3.05E−42 | −0.338123279 | 0.109 | 0.294 |
| DUSP6 | 9.13E−63 | −0.339265956 | 0.175 | 0.432 |
| TOB1 | 1.20E−56 | −0.34066125 | 0.341 | 0.632 |
| KRT19 | 1.97E−45 | −0.341191828 | 0.859 | 0.97 |
| RIMS1 | 5.73E−48 | −0.343999256 | 0.025 | 0.163 |
| EPB41L4A-AS1 | 2.93E−56 | −0.346969369 | 0.131 | 0.358 |
| CD82 | 1.64E−55 | −0.347784968 | 0.116 | 0.334 |
| SEMA3A | 2.19E−44 | −0.351118687 | 0.004 | 0.108 |
| NPPC | 2.89E−20 | −0.351827636 | 0.067 | 0.179 |
| SLC7A8 | 1.81E−41 | −0.353526706 | 0.015 | 0.137 |
| FAM3B | 2.12E−50 | −0.357779323 | 0.076 | 0.261 |
| KIF21A | 1.60E−67 | −0.359170467 | 0.109 | 0.343 |
| HLA-A | 2.45E−39 | −0.359489534 | 0.492 | 0.745 |
| RPS4X | 4.68E−53 | −0.360218837 | 0.86 | 0.977 |
| KRT7 | 4.30E−55 | −0.366550702 | 0.097 | 0.306 |
| MKL2 | 1.36E−70 | −0.37094929 | 0.109 | 0.353 |
| ALDH1A1 | 1.00E−59 | −0.373383124 | 0.339 | 0.642 |
| ALCAM | 2.31E−59 | −0.373633511 | 0.223 | 0.497 |
| ADRB2 | 6.18E−45 | −0.373762142 | 0.07 | 0.244 |
| PBX1 | 9.58E−71 | −0.373825431 | 0.1 | 0.335 |
| ZFP36L2 | 2.07E−66 | −0.380916942 | 0.37 | 0.689 |
| C6orf58 | 3.65E−56 | −0.381342515 | 0 | 0.113 |
| LGR6 | 3.44E−41 | −0.383105237 | 0.021 | 0.145 |
| SNHG8 | 3.47E−71 | −0.3844656 | 0.336 | 0.662 |
| ANXA1 | 5.85E−44 | −0.385024213 | 0.702 | 0.909 |
| CD74 | 4.43E−33 | −0.386159956 | 0.223 | 0.432 |
| PRDX6 | 1.02E−68 | −0.386952643 | 0.215 | 0.508 |
| CYP24A1 | 6.47E−33 | −0.387986129 | 0.019 | 0.124 |
| PIK3R1 | 1.42E−59 | −0.391893924 | 0.232 | 0.509 |
| HNMT | 4.98E−57 | −0.39370432 | 0.024 | 0.183 |
| NTS | 8.45E−26 | −0.400183798 | 0.217 | 0.398 |
| ALDH2 | 3.80E−72 | −0.403058078 | 0.168 | 0.453 |
| MT1X | 5.76E−85 | −0.409991909 | 0.417 | 0.78 |
| THSD4 | 2.28E−35 | −0.412737657 | 0.015 | 0.123 |
| STMN1 | 1.64E−31 | −0.412983683 | 0.079 | 0.231 |
| DUSP1 | 1.38E−43 | −0.413263471 | 0.748 | 0.92 |
| SLC23A2 | 9.87E−38 | −0.413904626 | 0.072 | 0.238 |
| ZFP36L1 | 1.94E−73 | −0.415589675 | 0.563 | 0.88 |
| CITED2 | 2.95E−52 | −0.424772745 | 0.113 | 0.334 |
| OGFRL1 | 2.00E−56 | −0.42550789 | 0.074 | 0.278 |
| EGFR | 5.30E−70 | −0.427252606 | 0.219 | 0.522 |
| IL18 | 2.11E−65 | −0.43018379 | 0.07 | 0.289 |
| PAX7 | 1.85E−61 | −0.431128826 | 0.019 | 0.179 |
| EXPH5 | 1.76E−62 | −0.431341729 | 0.134 | 0.39 |
| DUSP2 | 3.62E−51 | −0.440362222 | 0.162 | 0.402 |
| AQP5 | 7.58E−64 | −0.440666065 | 0.13 | 0.383 |
| KRT8 | 1.97E−39 | −0.448016598 | 0.387 | 0.646 |
| CHP2 | 1.58E−47 | −0.450669017 | 0.024 | 0.174 |
| AKR1C2 | 8.98E−62 | −0.451709257 | 0.039 | 0.222 |
| DSC3 | 2.23E−39 | −0.461304336 | 0.072 | 0.247 |
| C8orf4 | 2.74E−34 | −0.462942052 | 0.06 | 0.213 |
| CTSB | 6.27E−72 | −0.464271502 | 0.419 | 0.758 |
| MSMB | 1.30E−62 | −0.465009505 | 0.065 | 0.277 |
| ACAP2 | 4.11E−69 | −0.467575656 | 0.164 | 0.452 |
| TMPRSS11D | 2.73E−66 | −0.472801839 | 0.046 | 0.251 |
| NRCAM | 1.15E−45 | −0.481566248 | 0.007 | 0.124 |
| NET1 | 5.97E−72 | −0.488774228 | 0.132 | 0.411 |
| TGFB2 | 5.25E−41 | −0.498813482 | 0.067 | 0.242 |
| AQP3 | 1.80E−71 | −0.499781529 | 0.723 | 0.945 |
| CYP4B1 | 3.06E−62 | −0.500755612 | 0.13 | 0.392 |
| GDF15 | 8.89E−35 | −0.508378555 | 0.03 | 0.16 |
| XIST | 8.54E−79 | −0.508595051 | 0.177 | 0.489 |
| CA12 | 2.56E−74 | −0.510644198 | 0.087 | 0.341 |
| FMO3 | 1.64E−69 | −0.515639032 | 0.15 | 0.433 |
| UGT2A2 | 2.86E−57 | −0.52023835 | 0.305 | 0.61 |
| TSC22D1 | 4.03E−77 | −0.524449187 | 0.544 | 0.868 |
| ID2 | 4.47E−64 | −0.527196827 | 0.265 | 0.58 |
| DDIT4 | 3.76E−83 | −0.528231998 | 0.196 | 0.53 |
| TOP2A | 1.37E−14 | −0.53264994 | 0.03 | 0.103 |
| APOD | 6.96E−57 | −0.539689669 | 0.022 | 0.187 |
| AGR2 | 2.84E−97 | −0.541946064 | 0.403 | 0.799 |
| GLUL | 7.09E−94 | −0.564606638 | 0.507 | 0.874 |
| TMEM213 | 4.97E−62 | −0.570343566 | 0.01 | 0.165 |
| IGFBP5 | 5.20E−35 | −0.575368616 | 0.081 | 0.252 |
| LTF | 1.33E−98 | −0.596799501 | 0.012 | 0.235 |
| PTN | 3.69E−81 | −0.602805865 | 0.056 | 0.311 |
| S100A4 | 1.05E−61 | −0.630649333 | 0.127 | 0.394 |
| CLCA4 | 3.01E−57 | −0.652684858 | 0.019 | 0.184 |
| FAM107A | 6.39E−96 | −0.657375273 | 0.061 | 0.352 |
| ALDH3A1 | 4.58E−96 | −0.684368094 | 0.415 | 0.801 |
| SULT1E1 | 8.26E−63 | −0.706476775 | 0.056 | 0.28 |
| CSRP2 | 8.37E−92 | −0.718560608 | 0.071 | 0.373 |
| PIGR | 1.73E−77 | −0.732818924 | 0.047 | 0.293 |
| PTGS2 | 2.12E−57 | −0.775381343 | 0.021 | 0.191 |
| AKR1C3 | 6.16E−93 | −0.81164393 | 0.055 | 0.345 |
| BPIFB1 | 5.74E−153 | −0.847656499 | 0.1 | 0.529 |
| SCGB1A1 | 9.52E−73 | −1.016847797 | 0.021 | 0.221 |
| BPIFA1 | 3.26E−181 | −1.062184453 | 0.165 | 0.691 |
| EPAS1 | 2.28E−168 | −1.070083638 | 0.304 | 0.804 |
| FN1 | 4.99E−90 | −1.076738808 | 0.013 | 0.227 |
| ZG16B | 3.54E−194 | −1.11524002 | 0.013 | 0.41 |
| SPINK5 | 2.35E−112 | −1.192024686 | 0.121 | 0.495 |
| LYZ | 7.12E−231 | −1.425906395 | 0.03 | 0.521 |
| STATH | 0 | −2.560822125 | 0.029 | 0.855 |

In certain embodiments, the cell type may be detected by measuring one or more markers for each cell type selected from Table 1-15.

In another aspect, the present invention comprises a method for detecting type 2 inflammation, including chronic type 2, inflammation in a barrier tissue, comprising detecting loss of cell type diversity, including increase basal cell composition, by detecting one or more markers from Table 1-15.

In certain embodiments, the cell type as defined by expression of the markers described herein may be obtained by sorting cells based on expression of one or more markers for each cell type according to Table 1-15. In certain example embodiments, the quantity of cells may be determined by cell specific markers and gene expression assigned to each cell. In another aspect, the present invention comprises an isolated barrier cell characterized by expression of one or more markers from Table 1-15.

In another aspect, the present invention provides methods for detecting or quantifying barrier cells in a biological sample of a subject, the method comprising detecting or quantifying in the biological sample barrier cells as defined in any embodiment herein. The barrier cells may be detected or quantified using one or more cell surface markers for a cell type selected from Table 1-10.

In another aspect, the present invention provides for a method of isolating a barrier cell from a biological sample of a subject, the method comprising isolating from the biological sample barrier cells as defined as defined in any embodiment herein. The barrier cell may be isolated using one or more surface markers for a cell type selected from Table 1-10.

In certain embodiments, the barrier may be isolated, detected or quantified using a technique selected from the group consisting of RT-PCR, RNA-seq, single cell RNA-seq, western blot, ELISA, flow cytometry, mass cytometry, fluorescence activated cell sorting, fluorescence microscopy, affinity separation, magnetic cell separation, microfluidic separation, and combinations thereof.

In another aspect, the present invention provides for a method of modulating the barrier cell composition comprising treating a subject with an agent capable of targeting a barrier cell and inducing it to differentiate.

### IL-4/IL-13 Memory

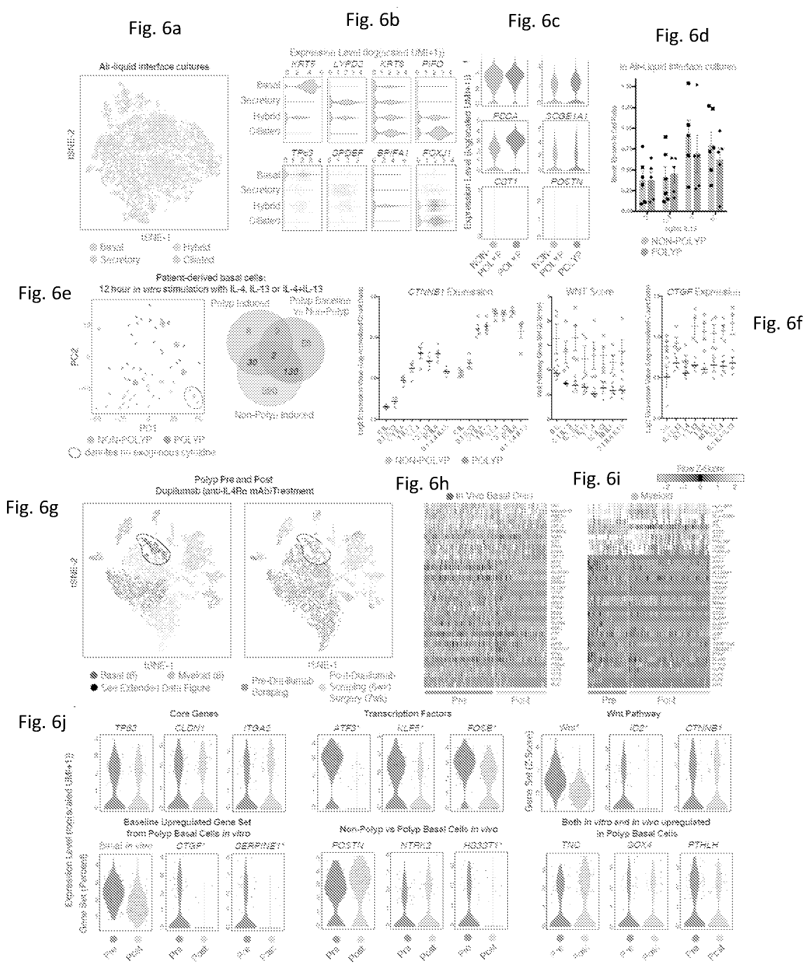

In another aspect, the present invention provides for a signature of “memory” of IL-4/IL-13 (FIG. 6E and Table 16). This signature is upregulated in polyp basal cells even after cytokines are no longer present. The signature is related to a IL-4/IL-13 induced signature. In certain embodiments, the “memory” signature is targeted for modulating barrier cells associated with disease. In certain embodiments, the memory signature is targeted with an antagonist of IL-4/IL-13 (e.g., dupilumab) as described further herein. In certain embodiments, the signature is further targeted by use of a modulating agent targeting one or more of the “memory” signature genes. In certain embodiments, an antagonist of IL-4/IL-13 may reduce expression of one or more memory genes induced by the cytokines, but during disease blocking IL-4/IL-13 signaling may not provide a therapeutic effect due to the persistence of one or more memory signature genes present in polyps independent of additional IL-4 and IL-13 activation of their receptors. Table 16 lists the genes present in FIG. 6E (Venn diagram). In certain embodiments, the memory signature comprises one or more genes that are 1) induced by IL-4/IL-13 in non-polyp, induced by IL-4/IL-13 in polyp and are baseline polyp genes; 2) induced by IL-4/IL-13 in non-polyp and are baseline polyp genes; or 3) induced by IL-4/IL-13 in polyp and are baseline polyp genes.

TABLE 16
nonpolyp_induced polyp_baseline_polyp_induced (2)
FAM3C, CTNNB1
nonpolyp_induced polyp_induced (30)
CDH3, MYO1B, HAS3, EHF, TNC, DKK3, SERPINB4, FABP5, TNFSF10, PTHLH, TBL1XR1, GJB2, CAV1,
THBS1, TNS4, SHISA9, LOC100132247, KRT18, LITAF, HSPH1, CCL26, CSF3, KRT8, SPHK1, HK1, CDH6,
SERPINB3, TP63, KRT23, CTSC
nonpolyp_induced polyp_baseline (130)
PHAX, LPP, PDE6A, MEFV, TVP23C, PLCXD1, PHACTR4, AP1S3, TRAF3IP2, PGAM5, SMYD4, UGGT1,
DFFA, UBB, LOC284454, IFITM3, TUBB2A, IRF1, AXL, SPN, RNF41, ANGPTL4, TUBB2B, NMNAT1,
TANGO2, HOOK3, S1PR2, HLA-H, LRTOMT, ZNF430, SCOC, ZNF793, CYP20A1, MDM2, KREMEN1,
ZC3H12D, FBLIM1, LOC646214, PDDC1, HLA-E, KRT6C, HYDIN2, COX18, PPM1K, MBOAT1, LINC00294,
ZNF526, P2RX5-TAX1BP3, GNE, NUDT19, KIF18B, PDE4C, LETM1, TLCD2, CHST6, METTL21A, IKZF3,
POTEE, HLA-G, XPNPEP3, TMEM33, MOG, TSPYL1, RBMS2, TAPBP, ANKRD20A9P, STON2, CYP4V2,
POTEM, OPA3, POLH, ZNF805, IFITM2, C17orf75, IRGQ, KCNQ1OT1, TAF8, UGDH-AS1, CCL5, NOL9,
CHP1, ORAI2, CA5B, HLA-B, LYZ, TOR1AIP2, TRAPPC2, SGTB, ZNF264, XIAP, RAMP2-AS1, SCAI,
ZBTB3, ZNF490, ORC4, DNAL1, FBXL18, CPT1A, TNFAIP8L1, LRRC57, RBM3, HLA-C, LOC148709,
LYRM7, ATCAY, PRICKLE2-AS3, AGMAT, NF2, LOC100131257, ACTG1, RUNDC1, MAVS, RPL36A-
HNRNPH2, PNPO, GDPD1, ILF3-AS1, LOC284023, PRR11, TMEM41B, ZBTB8A, TUBB4A, ZNF850, VHL,
IVD, FOXK1, MDM4, CCDC142, TRPV1, UBC, SENP5
polyp_baseline polyp_induced (2)
TM4SF1, IL1B
nonpolyp_induced (320)
HAUS2, GPR155, PIGX, TBC1D24, MCTS1, SPIB, GPR82, MOB3A, LOC100507173, SNHG16, GFOD2,
ZNF785, GNL3L, MPL, GLTP, IVNS1ABP, RAC1, NDUFV3, TUBA4A, CYCS, ME2, TRPM7, LOC727896,
FUT1, RBM34, SS18, SLC6A4, ZSWIM1, COTL1, TFDP2, NWD1, LEPREL4, PNPT1, NXN, SLC4A8, TSIX,
MREG, MS4A10, ZYG11B, AK3, FLVCR1, CRX, CXADR, CYP1A2, CCL22, MRI1, CABP4, SLC28A2, ABL2,
MAP1LC3C, ZYG11A, SCAMP4, EFNB1, CLCC1, DNAJC22, PNMA2, SNIP1, PXMP4, MPPE1, PALLD,
IAPP, WDR92, HNF1A-AS1, SLC35F6, ZFP14, FBXO27, FDPSL2A, BRIP1, RFT1, SLC5A5, OCLN, FAM73A,
SHROOM1, GATM-AS1, GOLGA3, BTG1, C1orf174, GEMIN8, ARHGAP1, CACNG8, TMEM136, METTL2B,
EXPH5, CORO2A, LOC286437, SLC25A32, GREB1, EMP2, MXRA7, OPHN1, DESI1, CEACAM22P,
LOC728558, FCF1, MFSD11, ENTPD4, DTX3L, METTL2A, TUBB6, LRRC58, GCLM, TMEM120B,
LOC100287792, SLC12A6, CARD8, ANAPC16, KRT16, CXorf56, PTRF, AP4S1, ASB6, RTCA, ZNF865,
C21orf62, SIX4, LOH12CR2, FKBP14, NME1-NME2, NPIPL3, SLC16A3, STK4, IGFBP7, MTDH, ZNF483,
LOC100506746, PACS2, MAPK13, SMU1, LINC00338, PLEKHG2, PRRG4, GLG1, TBXA2R, L2HGDH,
MRPS16, CBFA2T2, PTCHD4, ATP6V1G1, HP1BP3, C11orf58, NPHS1, CYP51A1, ANPEP, ENAH, FXN,
GSTM3, ADRA1A, INMT, IFNLR1, TMEM165, CYB5R3, SSR1, AFMID, ESYT2, LPIN3, TADA3, TPMT,
SLC35E2, ATP1A1, ARNTL2, LOC90834, MED18, IDS, PPP1CC, DDX51, ZNF626, KIN, RPS2, ZNF738,
SPATA5, ZNF587, TMEM212, AKAP5, C4orf19, ARGFX, DCAF10, ALDH1A3, EDARADD, PTAFR, CLSPN,
ZFAND5, EIF2S3, FAM122C, BHMT2, TM7SF3, MTFMT, C12orf65, LOXL4, QPCTL, AKIP1, SDE2, TRIM45,
SMIM12, VSIG1, LRRN4CL, SLFNL1-AS1, RELL1, ARPP19, ASTN2, RBBP5, GATAD1, DBT, JAK3, CD84,
FAM227A, TBC1D15, FBXL20, ENPP1, ULK2, SLC35E3, GTF2H2C, ADAMTS4, GJC1, GLT25D1, SLC50A1,
PEX13, CFLAR, RNF125, ADAT1, ZNF737, TTC39C, ICA1L, DAND5, ZSCAN29, FZD3, TUBB4B, CCBE1,
RAB27A, POU5F1, NT5DC3, EMX2OS, SPRED1, CPM, WHAMM, ATXN3, SHOX, GNG4, CAV2,
LOC643406, MTCH1, ZKSCAN3, FCAR, LOC100506688, GK5, ZNF714, WDR55, ODF2L, ZNF818P, ZNF814,
LOC284950, NLRP12, LRRC27, FKBP5, CNNM3, CBX5, RNF168, PPIEL, IBA57, DLEU2, PER2, HAUS3,
PCDH11X, MYLK3, PCBD2, STYX, LOC613037, NMT2, NUP43, LIMS1, TRIM16L, RABL5, APOL1,
TINAGL1, ZXDC, PDK3, CEP68, EEF2K, VPS53, TRMT2B, ZSCAN22, GTF2H3, IRAK4, ARSA,
LOC100506190, MIR143HG, ALG1, UCKL1-AS1, LRP10, APOBEC3F, WAC, SPATS2, UTP11L, LRPAP1,
ZNF829, MAPK1IP1L, SKA1, CEP104, PARD6B, TNFAIP8L2-SCNM1, KLRD1, CXorf38, LOC100505876,
SAR1B, UBE2Q2P1, C10orf32, LOC100289019, TRIM58, PDLIM5, RPS6KA3, MTPAP, PTGIS, SPAST,
KRT75, PSTPIP2, OR7D2, CD3EAP, SLC25A15, NLRC3, KCNA7, ABCC9
polyp_induced (8)
TGM2, CYP2S1, HS3ST1, TENM2, SLC1A5, SLC26A2, NABP1, SPINT2
polyp_baseline (53)
HLA-F, SOX4, MYL12B, BST2, SHC1, INHBA, IFI6, SQSTM1, LYPLA1, RAP1B, MT1L, ANXA8L1, PLTP,
NBPF10, MGST3, PLEKHB2, ICAM1, PHLDA1, PPP1CB, PAFAH1B2, SLC3A2, ANXA8L2, ISG20, GNG12,
TNFRSF10B, GRN, DSG3, FN1, IFI27, C6orf62, SERPINE1, BNIP3, HLA-L, AKIRIN1, SYPL1, EFNA1,
CDKN1A, TUBA1A, HLA-A, CCND2, CACUL1, RPS26, B2M, FKBP9L, AHNAK2, IFITM1, TFPI2, RPS4Y1,
SNAI2, KRT6A, SRP9, CTGF, PGK1

### Residual Signature

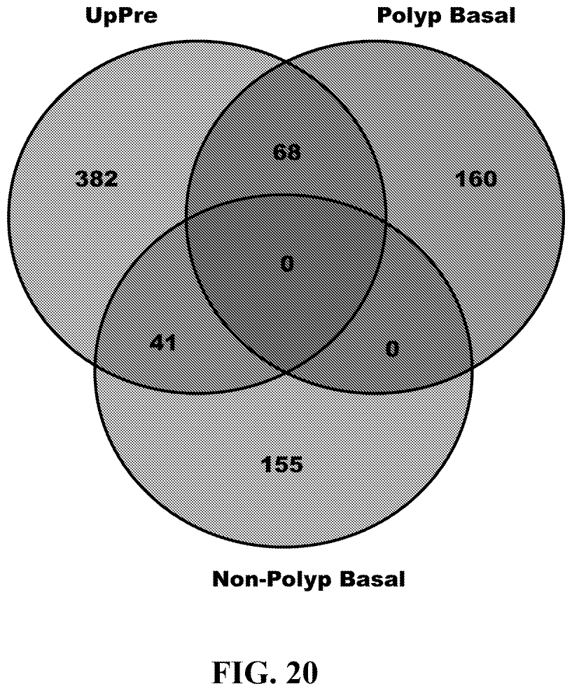

In another aspect, the present invention provides for a “residual” signature (FIG. 20 and Table 16 and Table 17). In certain embodiments, the residual signature comprises one or more genes present in polyp basal cells as compared to non-polyp cells and is not induced by cytokines (IL-4/IL-13). In certain embodiments, the residual signature comprises one or more genes not downregulated by an antagonist of IL-4/IL-13 (e.g., dupilumab), but that are differentially expressed between polyp vs non-polyp basal cells. In other words, the signature is differentially expressed between polyp and non-polyp and is cytokine independent. Thus, the signature differentiates between disease and non-disease. In certain embodiments, the residual signature is targeted for treatment of disease. In certain embodiments, the “residual” signature is targeted for modulating barrier cells associated with disease. In certain embodiments, the IL-4/IL-13 or memory signature is targeted with an antagonist of IL-4/IL-13 (e.g., dupilumab) in combination with an agent that modulates activity of one or more residual signatures genes associated with polyps. In certain embodiments, an antagonist of IL-4/IL-13 may reduce expression of genes induced by the cytokines, but disease may persist due to expression of the residual genes present in polyps. In certain embodiments, the residual signature comprises one or more genes that are 1) baseline polyp genes only and not induced by IL-4/IL-13 (Table 16, 53 genes); or 2) baseline polyp genes and not downregulated by an antagonist of IL-4/IL-13 (Table 17, 160 genes that are not downregulated by dupilumab, but differentiate polyp vs non-polyp basal cells). In certain embodiments, one or more of CD44, CTNNB1, POSTN, or TNC are targeted.

TABLE 17
Polyp Basal UpPre (68)
GPR155, PDPN, TMSB10, S100P, UBB, MTRNR2L2, MYL12B, NAMPT, ELOVL5, CYP2S1, CD55, HS3ST1,
LGALS3, PTHLH, SEC14L1, TACSTD2, THBS1, PHLDA1, BTF3, ETS2, PMAIP1, S100A2, ALOX15, CD99,
BHLHE40, MYL6, GSTP1, NABP1, KRT17, S100A10, KLF6, IFITM2, EMP1, ACTB, LAMB3, SERPINE1,
CEBPD, TM4SF1, GSN, SGK1, IL8, CCND2, IRAKI, ADM, YBX3, GPRC5A, CAV2, OAZ1, WNT4, PSMB3,
GPX4, NFKBIA, IGFBP3, SFN, RND3, CD9, LMNA, KLF10, NCOA7, HMGA1, SOCS3, TFF3, TNFRSF12A,
FXYD3, GCLC, MYADM, CBR1, RPS27
Non-Polyp Basal UpPre (41)
ANXA1, CITED2, UGT2A2, TMX4, BPIFB1, DNAJB1, DDIT4, TSC22D1, PRDX6, EPB41L4A-AS1, DUSP6,
SCGB1A1, ALDH3A2, EGFR, MSMB, CHD2, AQP3, ALDH3A1, DDIT3, TFCP2L1, GLUL, RPS4X, SPRY2,
ID2, WFDC2, ZFP36L2, KRT19, AQP5, FLRT3, BRD2, HLA-B, HLA-A, KRT8, DUSP1, LGALS8, ADRB2,
RNA18S5, JUN, RPL30, TOB1, CXCL17
UpPre (382)
RAB2A, FSTL1, RPS11, ATOX1, JAG2, POLR2G, BTG2, RN7SK, SLC25A25, DHCR24, GSR, HSPB1, NBN,
QARS, IER2, IVNS1ABP, CNN2, GAS5, LMO7, RAC1, PAK1, FOSL2, TOMM20, DDB1, PPP1R15A, RPL19,
EEF2, ID3, SERPINB5, ATP5G2, NDUFS1, MKNK2, SOD2, C7orf55-LUC7L2, SLC35F5, IRF1, SERPINH1,
CDK5RAP2, ANAPC11, CAPS, RPS5, SERINC3, RPL7A, SORL1, ARHGAP18, SRSF7, DDOST, EPS8, OPTN,
CCNL1, HERPUD1, GHITM, ATP5O, HMGN2, CLDN4, UGDH, MARCH7, TSKU, ETV3, PDLIM1,
PPP1R15B, HN1L, SQSTM1, CLTA, SNRNP70, ZCRB1, ATF3, RHOC, EID1, PLP2, SDF4, TAGLN2,
HNRNPF, DAZAP2, SNHG5, RAD23B, BTG1, TNPO1, RIPK4, CHST9, SOD1, EIF4A1, MAOA, MINK1,
TSPO, DLK2, SRI, MUC16, NR4A1, HLA-E, RPL15, AFG3L2, HDAC7, EIF1, DDX3Y, HBEGF, NDUFV1,
FOS, APH1A, JUNB, TUBB, CFL1, TIPARP, MAP3K13, SSH1, RHOB, RPLP1, PSMB6, ADI1, SCP2, GNG5,
BLCAP, ABHD2, RPS12, ID1, CSDE1, RPL28, SH3GLB1, ZNHIT1, PPAP2C, BCAM, ELF3, MDK, KLF5,
UBL5, HNRNPAB, TMEM59, SYNCRIP, UBA52, PLXNB1, GNB2L1, RPS3, F2R, RAI14, LRRC8A, TUBA1C,
CAPN1, TNS4, SPINT1, TMBIM6, TSC22D3, DLX5, PRDX1, KDM2A, H3F3B, KAL1, C16orf72, EZR, PLEC,
PPP1CB, FGFR3, HSP90AB1, ARF1, PAFAH1B2, SLC3A2, RPL18, IER5, CDKN2AIP, TMEM261, SYT8,
IRF6, CHD4, HSPA8, NDUFB4, DLL1, SYF2, IBTK, RAB34, MIDI, SLC9A3R1, MGST1, EGR1, LITAF,
LNX2, METTL7A, GDI2, AHNAK, RPS8, CYB5R3, PERP, KRT15, TUBA1B, RPL4, EIF4A3, PPP2CA, RPS21,
ARFGAP3, POLR2A, ATP1A1, MTSS1L, FTL, VAPA, TMEM14B, KLF4, DDX17, FLOT2, CDCA7L,
SH3BGRL, ARPC5, PPL, RPS2, UBR5, CDC42EP4, VMP1, LEPROT, TPT1, SOX9, CDC42SE1, FTH1, IER3,
FAU, TSPYL2, JUND, ZFP36, JUP, PHB2, TGFBR2, HNRNPA0, BAG5, PCBP1, ZFYVE21, ADH7, EGR2,
CIB1, NEDD4L, YPEL5, CDKN1A, FBXL3, RAB11A, SDC4, EIF3D, HSPA1B, CTNND1, ARHGDIA, NONO,
FGFR2, FLOT1, TOR1AIP2, PRKAR1A, SEPT10, NFKBIZ, SERTAD1, AJUBA, CSTB, TNFRSF1A, PPP2R5C,
HOOK1, USP22, EIF4H, SELM, RPL11, SEPW1, DNAJA1, MXD1, TUBB4B, RPL8, FZD6, PER1, RAB1A,
MUC5AC, UBE2D3, SF3B14, EIF3K, H3F3C, CDC42SE2, SLPI, REV1, RNA28S5, SMIM15, FOSB, KRT5,
C6orf48, PTP4A1, MIDN, SEMA5A, CTSK, RNF181, PRRC2C, EDF1, NUMA1, UBE3A, HSPA1A, NACA,
FKBP5, C11orf31, PSMB1, MIR22HG, CTSH, FAM107B, C14orf166, PIM3, OBSCN, FKBP9, DDR1, EIF3I,
PTPN14, F3, RBM3, MIR24-2, SNCA, PRDX5, CSNK1D, PPP1R10, EIF3F, TRIB1, NR1D2, FBXL5, HLA-C,
PER2, UQCRQ, GAPDH, ERRFI1, HN1, HPGD, SEC61B, STAT6, GPX1, PER3, S100A6, CEBPB, SSBP1,
MYC, WDR26, RPS14, S100A16, C9orf3, PHYHD1, C12orf57, ACTG1, CSNK1A1, SPCS1, ATP1B3,
GABARAPL2, RPLP2, KDSR, HINT1, KLF9, RPS24, RPL27, LAMA3, SPAG4, RPS16, ALDOA, SRSF3,
NDFIP1, FLNA, LAMA5, PEBP1, CYR61, EGR3, GCNT1, VPS37B, MAFF, ATF4, NCOA4, SIK1, SUOX,
ALAS1, STXBP3, MCL1, PPP4R1, MAT2A, RAP2B, HEBP2, CTGF, STARD7, MRFAP1, BLVRB, RAN,
ANXA11, LYPLAL1, ELK4, LIMK2, WEE1, CKS2, GADD45B, SF1, UBC, HSP90AA1, PPP1CA, OAT,
ANKRD37
Polyp Basal (160)
CD44, ANXA2, YBX1, EDN1, ARPC2, VAMP8, RPS18, EHF, HAS3, MBNL2, MTRNR2L9, CPXM2, NOS2,
HMGB3, TNC, IFITM3, TSPAN3, BIK, CXADR, LGALS7, SERPINF1, TPM4, SERINC5, IGHG4, SERPINE2,
NTRK2, PAPSS2, SDPR, ANXA3, RPL21, POSTN, CXCL2, MTRNR2L1, NDRG1, TPI1, SERPINB4, WIPI1,
AL353147.1, CKB, GCLM, LRRC17, FAM110C, RPL18A, CAV1, VAMP5, IGHA1, VSNL1, KCNE3,
SPARCL1, ARID5B, ARL4C, MTRNR2L3, CXCL1, IGHG1, CNN3, CST1, MYO10, IGHG3, AAMDC, HSPH1,
ENO1, VCL, SYNGR2, LAMTOR4, CCL26, TSLP, SERPINB2, TMEM123, TGIF1, IGHM, RPL7, CMYA5,
SH3BGRL3, LOXL4, MAP3K8, ANO1, PGAM1, STOM, MYL9, PLS1, CTNNAL1, EFNA1, MTRNR2L8,
RPL13A, PAWR, RPL41, LDLR, ITGB1, TCEB2, SERPINB10, NEDD9, SERPINB13, LDHA, DSE, DDX58,
GPC3, SERF2, KLHL5, MRPS6, ADAM9, CDCP1, EGLN3, SMARCAD1, CTNNB1, MIF, PFN1, CTD-
2228K2.5, CDH26, IGHA2, ST6GAL1, PLEKHA1, RPS25, GALNT7, CTD-2090I13.1, SNRPG, RPL17, SRGN,
ACTN1, MTRNR2L13, MORF4L1, RPSAP58, AREG, PLEKHA5, TMSB4X, IFITM1, SLC5A3, GEM, REXO2,
KCNJ16, RPS4Y1, MMP10, NRG1, TBC1D9, LBH, FBXO32, TXNDC17, PHLDB2, SLC6A14, KRT6A, CP,
TNFAIP3, CTD-2319112.1, SERTAD4-AS1, AMD1, F0SL1, LRRFIP1, IL6, UBBP4, BTG3, SLC2A1, IGJ,
TMA7, SSR4, FGFBP1, HSD17B13, CCDC51, SMOC2, SHISA5, CTSC, XPR1
Non-Polyp Basal (155)
COBLL1, PAX7, ACSS3, PTN, RTN4, TGFB2, SULT1E1, PRKAR2B, GDF15, OGFRL1, MAN1A1, LGR6,
CHD9, GADD45G, DSC3, SYNPO2, CHP2, PUM2, EPDR1, ARL6IP1, CLCA4, PSAP, GPNMB, BPIFA1,
MT1E, HNMT, SLC23A2, S100A4, HES1, SAMHD1, SPRY1, NFIB, FMO3, DHRS7, TBC1D5, ZFP36L1,
EXPH5, NBPF10, ALDH2, Clorf63, SEMA3A, CLEC2B, RARRES3, KBTBD3, CD82, C8orf4, ALCAM,
AZGP1, XIST, MLF1, DUSP2, HMGN3, CD74, FUS, HLA-DRA, APOD, EPHX1, TOP2A, PLEKHH1,
ALDH1A1, PIGR, CFH, MKL2, CTSL, LTF, SNHG8, JAG1, SNCG, NUPR1, NEBL, AKR1C2, ARSD, PARP14,
NPPC, HEY1, PBX1, FN1, KRT7, CYSTM1, PCLO, PIK3R3, SPINK5, IFI27, MT1G, TSPAN1, NAB1, OXCT1,
ERN2, C6orf58, CTSB, NDRG2, TMEM213, ACAP2, FAM3B, S100A14, LYZ, RARRES1, PIP, KIF21A,
SCNN1A, HSPA2, NRCAM, CSRP2, SVIP, PIK3R1, SLC38A2, FAM107A, PTGS2, GPX3, MALAT1, MT-CO3,
OXR1, HAS2, MTCH1, IL18, CCND1, SMARCA2, NET1, AKR1C3, ZNF750, RHOV, SREBF1, ZNF703,
EPAS1, THSD4, LRIG1, SNHG7, SCGB3A1, RIMS1, CTSD, TMPRSS11D, GCHFR, NTS, MT1X, MT2A, PNN,
FAM3D, ZG16B, MT-ND1, SLITRK6, CYP24A1, TXNIP, SLC7A8, CA12, STAT1, IGFBP5, STATH, CYP4B1,
IFI44, CRNDE, SAT1, AGR2, ABCA13, TRIM24, CLK1

### Detection of Cell Sub-Populations

In one embodiment, the method comprises detecting or quantifying epithelia cells in a biological sample obtained from the barrier tissue. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least 50%, or at least 60%, or at least 70%, or at least 80%, at least 90% or more, up to and including a 100% decrease (i.e., absent level as compared to a reference sample), or any decrease between 10-100% as compared to a reference level, as that.

The terms “sample” or “biological sample” as used throughout this specification include any biological specimen obtained from a subject. Particularly useful samples are those known to comprise, or expected or predicted to comprise gut cells as taught herein. Preferably, a sample may be readily obtainable by minimally invasive methods, such as blood collection or tissue biopsy, allowing the removal/isolation/provision of the sample from the subject (e.g., colonoscopy).

The terms “quantity”, “amount” and “level” are synonymous and generally well-understood in the art. The terms as used throughout this specification may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. Such values or ranges may be obtained as conventionally known.

An absolute quantity of a marker may be advantageously expressed as weight or as molar amount, or more commonly as a concentration, e.g., weight per volume or mol per volume. A relative quantity of a marker may be advantageously expressed as an increase or decrease or as a fold-increase or fold-decrease relative to said another value, such as relative to a reference value. Performing a relative comparison between first and second variables (e.g., first and second quantities) may but need not require determining first the absolute values of said first and second variables. For example, a measurement method may produce quantifiable readouts (such as, e.g., signal intensities) for said first and second variables, wherein said readouts are a function of the value of said variables, and wherein said readouts may be directly compared to produce a relative value for the first variable vs. the second variable, without the actual need to first convert the readouts to absolute values of the respective variables.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises 240%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The term “monitoring” generally refers to the follow-up of a disease or a condition in a subject for any changes which may occur over time.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

### Methods of Detection and Isolation of Cell Types Using Biomarkers

In certain embodiments, the cell types disclosed herein may be detected, quantified or isolated using a technique selected from the group consisting of flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH) and combinations thereof. The technique may employ one or more agents capable of specifically binding to one or more gene products expressed or not expressed by the gut cells, preferably on the cell surface of the gut cells. The one or more agents may be one or more antibodies. Other methods including absorbance assays and colorimetric assays are known in the art and may be used herein.

Depending on factors that can be evaluated and decided on by a skilled person, such as, inter alia, the type of a marker (e.g., peptide, polypeptide, protein, or nucleic acid), the type of the tested object (e.g., a cell, cell population, tissue, organ, or organism, e.g., the type of biological sample of a subject, e.g., whole blood, plasma, serum, tissue biopsy), the expected abundance of the marker in the tested object, the type, robustness, sensitivity and/or specificity of the detection method used to detect the marker, etc., the marker may be measured directly in the tested object, or the tested object may be subjected to one or more processing steps aimed at achieving an adequate measurement of the marker.

In other example embodiments, detection of a marker may include immunological assay methods, wherein the ability of an assay to separate, detect and/or quantify a marker (such as, preferably, peptide, polypeptide, or protein) is conferred by specific binding between a separable, detectable and/or quantifiable immunological binding agent (antibody) and the marker. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc.

In certain example embodiments, detection of a marker or signature may include biochemical assay methods, including inter alia assays of enzymatic activity, membrane channel activity, substance-binding activity, gene regulatory activity, or cell signaling activity of a marker, e.g., peptide, polypeptide, protein, or nucleic acid.

In other example embodiments, detection of a marker may include mass spectrometry analysis methods. Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), may be useful herein for separation, detection and/or quantification of markers (such as, preferably, peptides, polypeptides, or proteins). Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; atmospheric pressure photoionization mass spectrometry (APPI-MS); APPI-MS/MS; and APPI-(MS)n. Peptide ion fragmentation in tandem MS (MS/MS) arrangements may be achieved using manners established in the art, such as, e.g., collision induced dissociation (CID). Detection and quantification of markers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods.

In other example embodiments, detection of a marker may include chromatography methods. In a one example embodiment, chromatography refers to a process in which a mixture of substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography may be columnar. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilised metal affinity chromatography, and the like.

In certain embodiments, further techniques for separating, detecting and/or quantifying markers may be used in conjunction with any of the above described detection methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridization-based analysis, microarray expression analysis, digital gene expression profiling (DGE), RNA-in-situ hybridization (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like. By means of an example, methods to profile the RNA content of large numbers of individual cells have been recently developed. The cell of origin is determined by a cellular barcode. In certain embodiments, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10. 1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

The terms “isolating” or “purifying” as used throughout this specification with reference to a particular component of a composition or mixture (e.g., the tested object such as the biological sample) encompass processes or techniques whereby such component is separated from one or more or (substantially) all other components of the composition or mixture (e.g., the tested object such as the biological sample). The terms do not require absolute purity. Instead, isolating or purifying the component will produce a discrete environment in which the abundance of the component relative to one or more or all other components is greater than in the starting composition or mixture (e.g., the tested object such as the biological sample). A discrete environment may denote a single medium, such as for example a single solution, dispersion, gel, precipitate, etc. Isolating or purifying the specified cells from the tested object such as the biological sample may increase the abundance of the specified cells relative to all other cells comprised in the tested object such as the biological sample, or relative to other cells of a select subset of the cells comprised in the tested object such as the biological sample, e.g., relative to other white blood cells, peripheral blood mononuclear cells, immune cells, antigen presenting cells, or dendritic cells comprised in the tested object such as the biological sample. By means of example, isolating or purifying the specified cells from the tested object such as the biological sample may yield a cell population, in which the specified cells constitute at least 40% (by number) of all cells of said cell population, for example, at least 45%, preferably at least 50%, at least 55%, more preferably at least 60%, at least 65%, still more preferably at least 70%, at least 75%, even more preferably at least 80%, at least 85%, and yet more preferably at least 90%, at least 95%, at least 96%, at least 97%, at least 98%, at least 99%, or even 100% of all cells of said cell population.

The method may allow to detect or conclude the presence or absence of the specified cells in a tested object (e.g., in a cell population, tissue, organ, organism, or in a biological sample of a subject). The method may also allow to quantify the specified cells in a tested object (e.g., in a cell population, tissue, organ, organism, or in a biological sample of a subject). The quantity of the specified cells in the tested object such as the biological sample may be suitably expressed for example as the number (count) of the specified immune cells per standard unit of volume (e.g., ml, μl or nl) or weight (e.g., g or mg or ng) of the tested object such as the biological sample. The quantity of the specified cells in the tested object such as the biological sample may also be suitably expressed as a percentage or fraction (by number) of all cells comprised in the tested object such as the biological sample, or as a percentage or fraction (by number) of a select subset of the cells comprised in the tested object such as the biological sample, e.g., as a percentage or fraction (by number) of white blood cells, peripheral blood mononuclear cells, immune cells, antigen presenting cells, or dendritic cells comprised in the tested object such as the biological sample. The quantity of the specified cells in the tested object such as the biological sample may also be suitably represented by an absolute or relative quantity of a suitable surrogate analyte, such as a peptide, polypeptide, protein, or nucleic acid expressed or comprised by the specified cells.

Where a marker is detected in or on a cell, the cell may be conventionally denoted as positive (+) or negative (−) for the marker. Semi-quantitative denotations of marker expression in cells are also commonplace in the art, such as particularly in flow cytometry quantifications, for example, “dim” vs. “bright”, or “low” vs. “medium”/“intermediate” vs. “high”, or “−” vs. “+” vs. “++”, commonly controlled in flow cytometry quantifications by setting of the gates. Where a marker is quantified in or on a cell, absolute quantity of the marker may also be expressed for example as the number of molecules of the marker comprised by the cell.

Where a marker is detected and/or quantified on a single cell level in a cell population, the quantity of the marker may also be expressed as a percentage or fraction (by number) of cells comprised in said population that are positive for said marker, or as percentages or fractions (by number) of cells comprised in said population that are “dim” or “bright”, or that are “low” or “medium”/“intermediate” or “high”, or that are “−” or “+” or “++”. By means of an example, a sizeable proportion of the tested cells of the cell population may be positive for the marker, e.g., at least about 20%, at least about 40%, at least about 50%, at least about 60%, at least about 70%, at least about 80%, at least about 90%, at least about 95%, or up to 100%.

### Use of Specific Binding Agents for Detection

In certain embodiments, the aforementioned detection methods and techniques may employ agent(s) capable of specifically binding to one or more gene products, e.g., peptides, polypeptides, proteins, or nucleic acids, expressed or not expressed by the immune cells as taught herein. In certain preferred embodiments, such one or more gene products, e.g., peptides, polypeptides, or proteins, may be expressed on the cell surface of the immune cells (i.e., cell surface markers, e.g., transmembrane peptides, polypeptides or proteins, or secreted peptides, polypeptides or proteins which remain associated with the cell surface). Hence, further disclosed are binding agents capable of specifically binding to markers, such as genes or gene products, e.g., peptides, polypeptides, proteins, or nucleic acids as taught herein. Binding agents as intended throughout this specification may include inter alia antibodies, aptamers, spiegelmers (L-aptamers), photoaptamers, protein, peptides, peptidomimetics, nucleic acids such as oligonucleotides (e.g., hybridization probes or amplification or sequencing primers and primer pairs), small molecules, or combinations thereof.

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., KA in the order 1×109 M−1) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

Binding agents may be in various forms, e.g., lyophilised, free in solution, or immobilised on a solid phase. They may be, e.g., provided in a multi-well plate or as an array or microarray, or they may be packaged separately, individually, or in combination.

The term “specifically bind” as used throughout this specification means that an agent (denoted herein also as “specific-binding agent”) binds to one or more desired molecules or analytes (e.g., peptides, polypeptides, proteins, or nucleic acids) substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold, or at least about 1000-fold, or at least about 104-fold, or at least about 105-fold, or at least about 106-fold or more greater, than its affinity for a non-target molecule, such as for a suitable control molecule (e.g., bovine serum albumin, casein).

Preferably, the specific binding agent may bind to its intended target(s) with affinity constant (KA) of such binding KA≥1×106 M−1, more preferably KA≥1×107 M−1, yet more preferably KA≥1×108 M−1, even more preferably KA≥1×109 M−1, and still more preferably KA≥1×1010 M−1 or KA≥1×1011 M−1 or KA≥1×1012 M−1, wherein KA=[SBA_T]/[SBA][T], SBA denotes the specific-binding agent, T denotes the intended target. Determination of KA can be carried out by methods known in the art, such as for example, using equilibrium dialysis and Scatchard plot analysis.

In certain embodiments, the one or more binding agents may be one or more antibodies. As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunization, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo. Antibodies also encompasses chimeric, humanized and fully humanized antibodies.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

Nucleic acid binding agents, such as oligonucleotide binding agents, are typically at least partly antisense to a target nucleic acid of interest. The term “antisense” generally refers to an agent (e.g., an oligonucleotide) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein, such as hybridisation probes or amplification or sequencing primers and primer pairs) may typically be capable of annealing with (hybridizing to) the respective target nucleic acid sequences at high stringency conditions, and capable of hybridising specifically to the target under physiological conditions. The terms “complementary” or “complementarity” as used throughout this specification with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The reference to oligonucleotides may in particular but without limitation include hybridization probes and/or amplification primers and/or sequencing primers, etc., as commonly used in nucleic acid detection technologies. Binding agents as discussed herein may suitably comprise a detectable label. The term “label” refers to any atom, molecule, moiety or biomolecule that may be used to provide a detectable and preferably quantifiable read-out or property, and that may be attached to or made part of an entity of interest, such as a binding agent. Labels may be suitably detectable by for example mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes alone or in combination with moieties that may suppress or shift emission spectra by fluorescence resonance energy transfer (FRET).

In some embodiments, binding agents may be provided with a tag that permits detection with another agent (e.g., with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni2⁺), maltose:maltose binding protein, etc.

The marker-binding agent conjugate may be associated with or attached to a detection agent to facilitate detection. Examples of detection agents include, but are not limited to, luminescent labels; colourimetric labels, such as dyes; fluorescent labels; or chemical labels, such as electroactive agents (e.g., ferrocyanide); enzymes; radioactive labels; or radiofrequency labels. The detection agent may be a particle. Examples of such particles include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles may be colloidal gold particles.

In certain embodiments, the one or more binding agents are configured for use in a technique selected from the group consisting of flow cytometry, fluorescence activated cell sorting, mass cytometry, fluorescence microscopy, affinity separation, magnetic cell separation, microfluidic separation, and combinations thereof.

### Barrier Tissue Modulating Agents

A further aspect of the invention relates to a method for identifying an agent capable of modulating one or more phenotypic aspects of a barrier cell or barrier cell population as disclosed herein, comprising: a) applying a candidate agent to the cell or cell population; b) detecting modulation of one or more phenotypic aspects of the cell or cell population by the candidate agent, thereby identifying the agent.

In some aspects, it will be appreciated that the cell may be ex vivo or in vitro, it may be a host cell or cell line or progeny thereof, or it may be as part of an organoid such as an organ-on-a-chip. In some aspects, the cell may be in a sample taken from a patient, for example taken via a biopsy or other tissue sampling technique.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a gut cell or gut cell population may be modulated without substantially altering other (unintended, undesired) phenotypic aspect(s).

The term “barrier tissue modulating agent” broadly encompasses any condition, substance or agent capable of modulating one or more phenotypic aspects of a barrier cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a barrier cell or barrier cell population as disclosed herein in a method comprising applying the candidate agent to the barrier cell or barrier tissue cell population (e.g., exposing the barrier cell or barrier tissue cell population to the candidate agent or contacting the barrier cell or barrier cell population with the candidate agent) and observing whether the desired modulation takes place.

Modulating agents may include any potential class of biologically active conditions, substances or agents, such as for instance antibodies, proteins, peptides, nucleic acids, oligonucleotides, small molecules, or combinations thereof.

By means of example but without limitation, agents can include low molecular weight compounds, but may also be larger compounds, or any organic or inorganic molecule effective in the given situation, including modified and unmodified nucleic acids such as antisense nucleic acids, RNAi, such as siRNA or shRNA, CRISPR/Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments, the agent is a small molecule having a chemical moiety. Agents can be known to have a desired activity and/or property, or can be selected from a library of diverse compounds.

### Antibodies

In some embodiments, the agent may be an antibody or fragment thereof. The term “antibody” (e.g., anti-KLRB1 or anti-CLEC2D antibody) is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

In some embodiments, the antibody is a humanized or chimeric antibody. “Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

As used herein, a “blocking” antibody or an antibody “antagonist” is one which inhibits or reduces biological activity of the antigen(s) it binds. For example, an antagonist antibody may bind KLRB1 or CLEC2D and inhibit their ability to interact. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Polypeptide-Based Modulating Agents

In certain embodiments, an agent may be soluble polypeptide, a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-y, IL-2, IL-3, IL-4, IL-6, granulocyte-macrophage colony-stimulating factor (GM-CSF), interferon-y, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-a, interferon-3, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signalling molecules, such as tumour necrosis factor (TNF) and interferons (e.g., interferon-a, interferon-3, interferon-y, interferon-k, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Small Molecule Modulating Agents

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015. In certain aspects of this invention, the culture of the invention is used to grow and differentiate a cachectic target cell to play the role of test cells for standard drug screening and toxicity assays. Assessment of the activity of candidate pharmaceutical compounds generally involves combining the target cell (e.g., a myocyte, an adipocyte, a cardiomyocyte or a hepatocyte) with the candidate compound, determining any change in the morphology, marker phenotype, or metabolic activity of the cells that is attributable to the candidate compound (compared with untreated cells or cells treated with an inert compound, such as vehicle), and then correlating the effect of the candidate compound with the observed change. The screening may be done because the candidate compound is designed to have a pharmacological effect on the target cell, or because a candidate compound may have unintended side effects on the target cell. Alternatively, libraries can be screened without any predetermined expectations in hopes of identifying compounds with desired effects.

Cytotoxicity can be determined in the first instance by the effect on cell viability and morphology. In certain embodiments, toxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT.

Additional further uses of the culture of the invention include, but are not limited to, its use in research e.g., to elucidate mechanisms leading to the identification of novel targets for therapies, and to generate genotype-specific cells for disease modeling, including the generation of new therapies customized to different genotypes. Such customization can reduce adverse drug effects and help identify therapies appropriate to the patient's genotype.

In certain embodiments, the present invention provides method for high-throughput screening. “High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus, “high-throughput screening” as used herein does not include handling large quantities of radioactive materials, slow and complicated operator-dependent screening steps, and/or prohibitively expensive reagent costs, etc.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures of the present invention may be used to screen for drugs that induce or reduce the signature in immune cells as described herein. The signature may be used for GE-HTS (Gene Expression-based High-Throughput Screening). In certain embodiments, pharmacological screens may be used to identify drugs that selectively activate gut cells.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature of the present invention in silico.

### Genetic Modification

In certain embodiments, the one or more modulating agents may be a genetic modifying agent. The genetic modifying agent may comprise a CRISPR system, a zinc finger nuclease system, a TALEN, or a meganuclease.

In certain embodiments, one or more endogenous genes may be modified using a nuclease. The term “nuclease” as used herein broadly refers to an agent, for example a protein or a small molecule, capable of cleaving a phosphodiester bond connecting nucleotide residues in a nucleic acid molecule. In some embodiments, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some embodiments, a nuclease may recognize a single stranded target site, in other embodiments a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 5′ end of the respective DNA strand.

The nuclease may introduce one or more single-strand nicks and/or double-strand breaks in the endogenous gene, whereupon the sequence of the endogenous gene may be modified or mutated via non-homologous end joining (NHEJ) or homology-directed repair (HDR).

In certain embodiments, the nuclease may comprise (i) a DNA-binding portion configured to specifically bind to the endogenous gene and (ii) a DNA cleavage portion. Generally, the DNA cleavage portion will cleave the nucleic acid within or in the vicinity of the sequence to which the DNA-binding portion is configured to bind.

In certain embodiments, the DNA-binding portion may comprise a zinc finger protein or DNA-binding domain thereof, a transcription activator-like effector (TALE) protein or DNA-binding domain thereof, or an RNA-guided protein or DNA-binding domain thereof.

Programmable nucleic acid-modifying agents in the context of the present invention may be used to modify endogenous cell DNA or RNA sequences, including DNA and/or RNA sequences encoding the target genes and target gene products disclosed herein. In certain example embodiments, the programmable nucleic acid-modifying agents may be used to edit a target sequence to restore native or wild-type functionality. In certain other embodiments, the programmable nucleic-acid modifying agents may be used to insert a new gene or gene product to modify the phenotype of target cells. In certain other example embodiments, the programmable nucleic-acid modifying agents may be used to delete or otherwise silence the expression of a target gene or gene product. Programmable nucleic-acid modifying agents may used in both in vivo an ex vivo applications disclosed herein.

1. CRISPR/Cas Systems

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein His A, CorU.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is −4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordj ournals.org/content/34/7/e53. short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csxl, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

a) DNA Repair and NHEJ

In certain embodiments, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving by the CRISPR/Cas system can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which the CRISPR/Cas system generates a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with CRISPR/Cas system nickases induce two single strand breaks for the purpose of inducing NHEJ-mediated indels, two guide RNAs may be configured to position two single-strand breaks to provide for NHEJ repair a nucleotide of the target position.

b) dCas and Functional Effectors

Unlike CRISPR-Cas-mediated gene knockout, which permanently eliminates expression by mutating the gene at the DNA level, CRISPR-Cas knockdown allows for temporary reduction of gene expression through the use of artificial transcription factors. Mutating key residues in cleavage domains of the Cas protein results in the generation of a catalytically inactive Cas protein. A catalytically inactive Cas protein complexes with a guide RNA and localizes to the DNA sequence specified by that guide RNA's targeting domain, however, it does not cleave the target DNA. Fusion of the inactive Cas protein to an effector domain also referred to herein as a functional domain, e.g., a transcription repression domain, enables recruitment of the effector to any DNA site specified by the guide RNA.

In general, the positioning of the one or more functional domain on the inactivated CRISPR/Cas protein is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., Fok1) will be advantageously positioned to cleave or partially cleave the target. This may include positions other than the N-/C-terminus of the CRISPR protein.

In certain embodiments, Cas protein may be fused to a transcriptional repression domain and recruited to the promoter region of a gene. Especially for gene repression, it is contemplated herein that blocking the binding site of an endogenous transcription factor would aid in downregulating gene expression.

In an embodiment, a guide RNA molecule can be targeted to a known transcription response elements (e.g., promoters, enhancers, etc.), a known upstream activating sequences, and/or sequences of unknown or known function that are suspected of being able to control expression of the target DNA. Idem: adapt to refer to regions with the motifs of interest

In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

c) Guide Molecules

As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2,4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the compelementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programming of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10. 1038/naturel4592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID 1-GAI based system inducible by Gibberellin (GA) (see, e.g., www.nature.com/nchembio/journal/v8/n5/full/nchembio. 922.html).

A chemical inducible system can be an estrogen receptor (ER) based system inducible by 4-hydroxytamoxifen (4OHT) (see, e.g., www.pnas. org/content/104/3/1027. abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1.is and 500 milliseconds, preferably between 1.is and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extented length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide moleucle, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi: 10/1038/naturel9802 and Abudayyeh et al. “C2c2 is a single-component programmable RNA-guided RNA targeting CRISPR effector” bioRxiv doi: 10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Naturel2466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure™, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1): 102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91(2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/naturel5521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokIl Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

2. Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 201 1; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

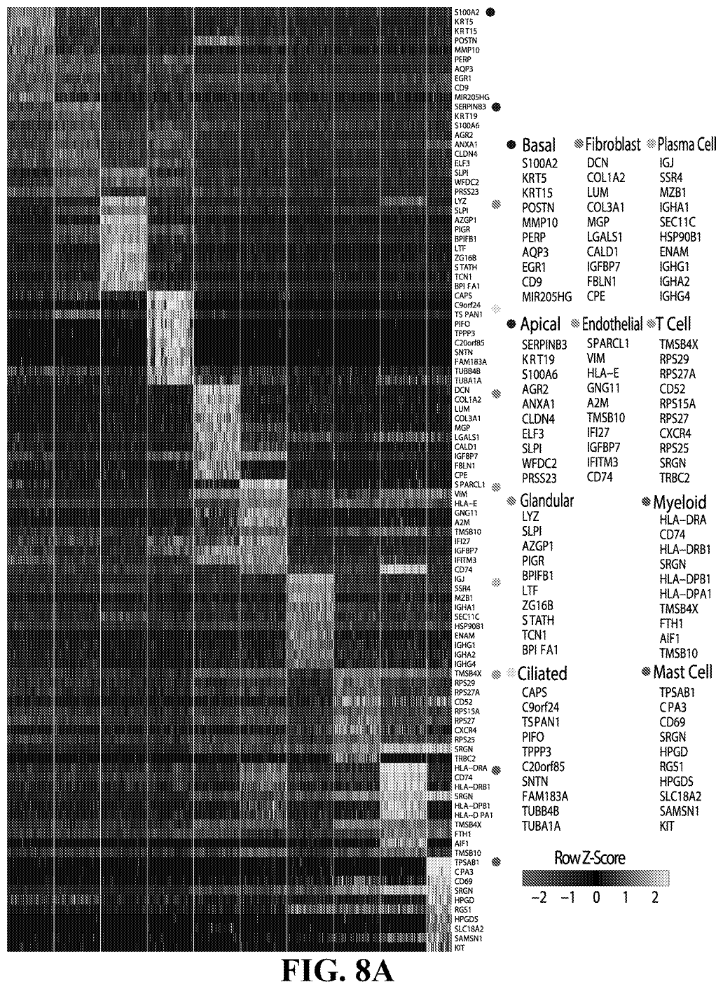

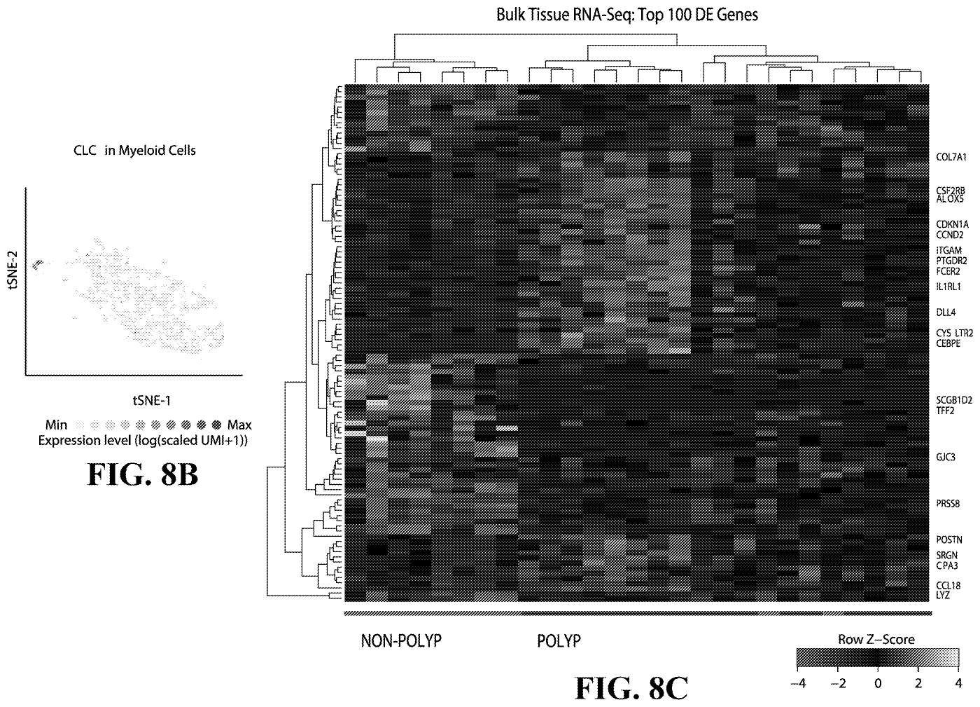

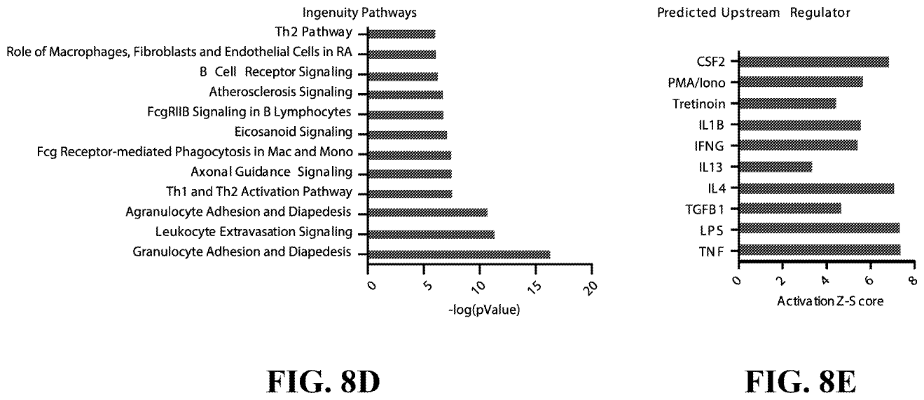

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 2)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

3. ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

4. Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### Delivery of Modulating Agents

The programmable nucleic acid modifying agents and other modulating agents, or components thereof, or nucleic acid molecules thereof (including, for instance HDR template), or nucleic acid molecules encoding or providing components thereof, may be delivered by a delivery system herein described.

Viral Delivery

Vector delivery, e.g., plasmid, viral delivery: the chromatin 3D structure modulating agents, can be delivered using any suitable vector, e.g., plasmid or viral vectors, such as adeno associated virus (AAV), lentivirus, adenovirus or other viral vector types, or combinations thereof. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

Compositions comprising a Cas effector module, complex or system comprising multiple guide RNAs, preferably tandemly arranged, or the polynucleotide or vector encoding or comprising said Cas effector module, complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. Use of a Cas effector module CRISPR system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible Cas effector module activator allows one to induce transcription right before the screen and therefore minimizes the chance of false negative hits. Accordingly, by use of the instant invention in screening, e.g., gain of function screens, the chance of false negative results may be minimized.

In another aspect, the invention provides an engineered, non-naturally occurring vector system comprising one or more vectors comprising a first regulatory element operably linked to the multiple Cas effector module CRISPR system guide RNAs that each specifically target a DNA molecule encoding a gene product and a second regulatory element operably linked coding for a CRISPR protein. Both regulatory elements may be located on the same vector or on different vectors of the system. The multiple guide RNAs target the multiple DNA molecules encoding the multiple gene products in a cell and the CRISPR protein may cleave the multiple DNA molecules encoding the gene products (it may cleave one or both strands or have substantially no nuclease activity), whereby expression of the multiple gene products is altered; and, wherein the CRISPR protein and the multiple guide RNAs do not naturally occur together. In a preferred embodiment the CRISPR protein is a Cas effector module, optionally codon optimized for expression in a eukaryotic cell. In a preferred embodiment the eukaryotic cell is a mammalian cell, a plant cell or a yeast cell and in a more preferred embodiment the mammalian cell is a human cell. In a further embodiment of the invention, the expression of each of the multiple gene products is altered, preferably decreased.

In one aspect, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas effector module complexed with the one or more guide sequence(s) that is hybridized to the one or more target sequence(s); and (b) a second regulatory element operably linked to an enzyme-coding sequence encoding said Cas effector module, preferably comprising at least one nuclear localization sequence and/or at least one NES; wherein components (a) and (b) are located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the CRISPR complex comprises one or more nuclear localization sequences and/or one or more NES of sufficient strength to drive accumulation of said CRISPR complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the Cas effector module, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, a host cell is transiently or non-transiently transfected with one or more vectors comprising the polynucleotides encoding the Cas effector module, system or complex for use in multiple targeting as defined herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art and exemplidied herein elsewhere. Cell lines are available from a variety of sources known to those with skill in the art (see, e.g., the American Type Culture Collection (ATCC) (Manassas, Va.)). In some embodiments, a cell transfected with one or more vectors comprising the polynucleotides encoding the Cas effector module, system or complex for use in multiple targeting as defined herein is used to establish a new cell line comprising one or more vector-derived sequences. In some embodiments, a cell transiently transfected with the components of a Cas effector module. system or complex for use in multiple targeting as described herein (such as by transient transfection of one or more vectors, or transfection with RNA), and modified through the activity of a Cas effector module, system or complex, is used to establish a new cell line comprising cells containing the modification but lacking any other exogenous sequence. In some embodiments, cells transiently or non-transiently transfected with one or more vectors comprising the polynucleotides encoding Cas effector module, system or complex for use in multiple targeting as defined herein, or cell lines derived from such cells are used in assessing one or more test compounds.

The term “regulatory element” is as defined herein elsewhere.

Advantageous vectors include lentiviruses and adeno-associated viruses, and types of such vectors can also be selected for targeting particular types of cells.

In one aspect, the invention provides a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the CRISPR complex to the respective target sequence(s) in a eukaryotic cell, wherein the CRISPR complex comprises a Cas effector module complexed with the one or more guide sequence(s) that is hybridized to the respective target sequence(s); and/or (b) a second regulatory element operably linked to an enzyme-coding sequence encoding said Cas effector module comprising preferably at least one nuclear localization sequence and/or NES. In some embodiments, the host cell comprises components (a) and (b). In some embodiments, component (a), component (b), or components (a) and (b) are stably integrated into a genome of the host eukaryotic cell. In some embodiments, component (a) further comprises two or more guide sequences operably linked to the first regulatory element, and optionally separated by a direct repeat, wherein when expressed, each of the two or more guide sequences direct sequence specific binding of a CRISPR complex to a different target sequence in a eukaryotic cell. In some embodiments, the Cas effector module comprises one or more nuclear localization sequences and/or nuclear export sequences or NES of sufficient strength to drive accumulation of said CRISPR enzyme in a detectable amount in and/or out of the nucleus of a eukaryotic cell.

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

In certain aspects the invention involves vectors. A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s) (e.g., sgRNAs); and, when a single vector provides for more than 16 RNA(s) (e.g., sgRNAs), one or more promoter(s) can drive expression of more than one of the RNA(s) (e.g., sgRNAs), e.g., when there are 32 RNA(s) (e.g., sgRNAs), each promoter can drive expression of two RNA(s) (e.g., sgRNAs), and when there are 48 RNA(s) (e.g., sgRNAs), each promoter can drive expression of three RNA(s) (e.g., sgRNAs). By simple arithmetic and well established cloning protocols and the teachings in this disclosure one skilled in the art can readily practice the invention as to the RNA(s) (e.g., sgRNA(s) for a suitable exemplary vector such as AAV, and a suitable promoter such as the U6 promoter, e.g., U6-sgRNAs. For example, the packaging limit of AAV is ˜4.7 kb. The length of a single U6-sgRNA (plus restriction sites for cloning) is 361 bp. Therefore, the skilled person can readily fit about 12-16, e.g., 13 U6-sgRNA cassettes in a single vector. This can be assembled by any suitable means, such as a golden gate strategy used for TALE assembly (www.genome-engineering.org/taleffectors/). The skilled person can also use a tandem guide strategy to increase the number of U6-sgRNAs by approximately 1.5 times, e.g., to increase from 12-16, e.g., 13 to approximately 18-24, e.g., about 19 U6-sgRNAs. Therefore, one skilled in the art can readily reach approximately 18-24, e.g., about 19 promoter-RNAs, e.g., U6-sgRNAs in a single vector, e.g., an AAV vector. A further means for increasing the number of promoters and RNAs, e.g., sgRNA(s) in a vector is to use a single promoter (e.g., U6) to express an array of RNAs, e.g., sgRNAs separated by cleavable sequences. And an even further means for increasing the number of promoter-RNAs, e.g., sgRNAs in a vector, is to express an array of promoter-RNAs, e.g., sgRNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53. short, www.nature.com/mt/journal/v16/n9/abs/mt2008144a.html). In an advantageous embodiment, AAV may package U6 tandem sgRNA targeting up to about 50 genes. Accordingly, from the knowledge in the art and the teachings in this disclosure the skilled person can readily make and use vector(s), e.g., a single vector, expressing multiple RNAs or guides or sgRNAs under the control or operatively or functionally linked to one or more promoters-especially as to the numbers of RNAs or guides or sgRNAs discussed herein, without any undue experimentation.

The guide RNA(s), e.g., sgRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the j3-actin promoter, the phosphoglycerol kinase (PGK) promoter, and the EF1α promoter. An advantageous promoter is the promoter is U6.

Aspects of the invention relate to bicistronic vectors for guide RNA and (optionally modified or mutated) Cas effector modules. Bicistronic expression vectors for guide RNA and (optionally modified or mutated) CRISPR enzymes are preferred. In general and particularly in this embodiment (optionally modified or mutated) CRISPR enzymes are preferably driven by the CBh promoter. The RNA may preferably be driven by a Pol III promoter, such as a U6 promoter. Ideally the two are combined.

In some embodiments, a loop in the guide RNA is provided. This may be a stem loop or a tetra loop. The loop is preferably GAAA, but it is not limited to this sequence or indeed to being only 4 bp in length. Indeed, preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). With regards to regulatory sequences, mention is made of U.S. patent application Ser. No. 10/491,026, the contents of which are incorporated by reference herein in their entirety. With regards to promoters, mention is made of PCT publication WO 2011/028929 and U.S. application Ser. No. 12/511,940, the contents of which are incorporated by reference herein in their entirety.

Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments of the invention may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

Typically, in the context of an endogenous nucleic acid-targeting system, formation of a nucleic acid-targeting complex (comprising a guide RNA hybridized to a target sequence and complexed with one or more nucleic acid-targeting effector modules) results in cleavage of one or both RNA strands in or near (e.g. within 1, 2, 3, 4, 5, 6, 7, 8, 9, 10, 20, 50, or more base pairs from) the target sequence. In some embodiments, one or more vectors driving expression of one or more elements of a nucleic acid-targeting system are introduced into a host cell such that expression of the elements of the nucleic acid-targeting system direct formation of a nucleic acid-targeting complex at one or more target sites. For example, a nucleic acid-targeting effector module and a guide RNA could each be operably linked to separate regulatory elements on separate vectors. Alternatively, two or more of the elements expressed from the same or different regulatory elements, may be combined in a single vector, with one or more additional vectors providing any components of the nucleic acid-targeting system not included in the first vector. nucleic acid-targeting system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a nucleic acid-targeting effector module and a guide RNA embedded within one or more intron sequences (e.g. each in a different intron, two or more in at least one intron, or all in a single intron). In some embodiments, the nucleic acid-targeting effector module and guide RNA are operably linked to and expressed from the same promoter.

Ways to package inventive Cpf1 coding nucleic acid molecules, e.g., DNA, into vectors, e.g., viral vectors, to mediate genome modification in vivo may include:
To achieve NHEJ-mediated gene knockout:
Single virus vector:
Vector containing two or more expression cassettes:
Promoter-Cpf1 coding nucleic acid molecule-terminator
Promoter-gRNA1-terminator
Promoter-gRNA2-terminator
Promoter-gRNA(N)-terminator (up to size limit of vector)
Double virus vector:
Vector 1 containing one expression cassette for driving the expression of Cpf1
Promoter-Cpf1 coding nucleic acid molecule-terminator
Vector 2 containing one more expression cassettes for driving the expression of one or more guideRNAs
Promoter-gRNA1-terminator
Promoter-gRNA(N)-terminator (up to size limit of vector)
To mediate homology-directed repair.
In addition to the single and double virus vector approaches described above, an additional vector can be used to deliver a homology-direct repair template.

The promoter used to drive Cpf1 coding nucleic acid molecule expression can include:
AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of Cpf1.
For ubiquitous expression, promoters that can be used include: CMV, CAG, CBh, PGK, SV40, Ferritin heavy or light chains, etc.

For brain or other CNS expression, can use promoters: SynapsinI for all neurons, CaMKIIalpha for excitatory neurons, GAD67 or GAD65 or VGAT for GABAergic neurons, etc.

For liver expression, can use Albumin promoter.

For lung expression, can use use SP-B.

For endothelial cells, can use ICAM.

For hematopoietic cells can use IFNbeta or CD45.

For Osteoblasts can one can use the OG-2.

The promoter used to drive guide RNA can include:
Pol III promoters such as U6 or H1
Use of Pol II promoter and intronic cassettes to express gRNA

Adeno Associated Virus (AAV)

Cpf1 and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of Cpf1 can be driven by a cell-type specific promoter. For example, liver-specific expression might use the Albumin promoter and neuron-specific expression (e.g. for targeting CNS disorders) might use the Synapsin I promoter.

In terms of in vivo delivery, AAV is advantageous over other viral vectors for a couple of reasons:
Low toxicity (this may be due to the purification method not requiring ultra centrifugation of cell particles that can activate the immune response) and
Low probability of causing insertional mutagenesis because it doesn't integrate into the host genome.

AAV has a packaging limit of 4.5 or 4.75 Kb. This means that Cpf1 as well as a promoter and transcription terminator have to be all fit into the same viral vector. Constructs larger than 4.5 or 4.75 Kb will lead to significantly reduced virus production. SpCas9 is quite large, the gene itself is over 4.1 Kb, which makes it difficult for packing into AAV. Therefore embodiments of the invention include utilizing homologs of Cpf1 that are shorter. For example:

| Species | Cas9 Size (nt) |
|---|---|
| Corynebacter diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Campylobacter jejuni | 2952 |
| Streptococcus thermophilus LMD-9 | 3396 |

rAAV vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

These species are therefore, in general, preferred Cpf1 species.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually. A tabulation of certain AAV serotypes as to these cells (see Grimm, D. et al, J. Virol. 82: 5887-5911 (2008)) is as follows:

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

Lentivirus

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMEM (serum-free) media and transfection was done 4 hours later. Cells were transfected with 10 μg of lentiviral transfer plasmid (pCasES 10) and the following packaging plasmids: 5 μg of pMD2.G (VSV-g pseudotype), and 7.5 ug of psPAX2 (gag/pol/rev/tat). Transfection was done in 4 mL OptiMEM with a cationic lipid delivery agent (50 uL Lipofectamine 2000 and 100 ul Plus reagent). After 6 hours, the media was changed to antibiotic-free DMEM with 10% fetal bovine serum. These methods use serum during cell culture, but serum-free methods are preferred.

Lentivirus may be purified as follows. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 ul of DMEM overnight at 4 C. They were then aliquotted and immediately frozen at −80° C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285). In another embodiment, RetinoStat®, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et al., HUMAN GENE THERAPY 23:980-991 (September 2012)) and this vector may be modified for the CRISPR-Cas system of the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the CRISPR-Cas system of the present invention. A minimum of 2.5×106 CD34+ cells per kilogram patient weight may be collected and prestimulated for 16 to 20 hours in X-VIVO 15 medium (Lonza) containing 2 μmol/L-glutamine, stem cell factor (100 ng/ml), Flt-3 ligand (Flt-3L) (100 ng/ml), and thrombopoietin (10 ng/ml) (CellGenix) at a density of 2×106 cells/ml. Prestimulated cells may be transduced with lentiviral at a multiplicity of infection of 5 for 16 to 24 hours in 75-cm2 tissue culture flasks coated with fibronectin (25 mg/cm2) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015.

Use of Minimal Promoters

The present application provides a vector for delivering an effector protein and at least one CRISPR guide RNA to a cell comprising a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the vector is an AAV vector. In another embodiment, the effector protein is a CRISPR anzyme. In a further embodiment, the CRISPR enzyme is SaCas9, Cpf1, Cas13b or C2c2.

In a related aspect, the invention provides a lentiviral vector for delivering an effector protein and at least one CRISPR guide RNA to a cell comprising a promoter operably linked to a polynucleotide sequence encoding Cpf1 and a second promoter operably linked to a polynucleotide sequence encoding at least one guide RNA, wherein the polynucleotide sequences are in reverse orientation.

In another aspect, the invention provides a method of expressing an effector protein and guide RNA in a cell comprising introducing the vector according any of the vector delivery systems disclosed herein. In an embodiment of the vector for delivering an effector protein, the minimal promoter is the Mecp2 promoter, tRNA promoter, or U6. In a further embodiment, the minimal promoter is tissue specific.

Dosage of Vectors

In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×105 particles (also referred to as particle units, pu) of adenoviral vector. In an embodiment herein, the dose preferably is at least about 1×106 particles (for example, about 1×106-1×1012 particles), more preferably at least about 1×107 particles, more preferably at least about 1×108 particles (e.g., about 1×108-1×1011 particles or about 1×108-1×1012 particles), and most preferably at least about 1×100 particles (e.g., about 1×109-1×1010 particles or about 1×109-1×1012 particles), or even at least about 1×1010 particles (e.g., about 1×1010-1×1012 particles) of the adenoviral vector. Alternatively, the dose comprises no more than about 1×1014 particles, preferably no more than about 1×1013 particles, even more preferably no more than about 1×1012 particles, even more preferably no more than about 1×1011 particles, and most preferably no more than about 1×1010 particles (e.g., no more than about 1×109 articles). Thus, the dose may contain a single dose of adenoviral vector with, for example, about 1×106 particle units (pu), about 2×106 pu, about 4×106 pu, about 1×107 pu, about 2×107 pu, about 4×107 pu, about 1×108 pu, about 2×108 pu, about 4×108 pu, about 1×109 pu, about 2×109 pu, about 4×109 pu, about 1×1010 pu, about 2×1010 pu, about 4×1010 pu, about 1×1011 pu, about 2×1011 pu, about 4×1011 pu, about 1×1012 pu, about 2×1012 pu, or about 4×1012 pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×1010 to about 1×1010 functional AAV/ml solution. The dosage may be adjusted to balance the therapeutic benefit against any side effects. In an embodiment herein, the AAV dose is generally in the range of concentrations of from about 1×105 to 1×1050 genomes AAV, from about 1×108 to 1×1020 genomes AAV, from about 1×1010 to about 1×1016 genomes, or about 1×1011 to about 1×1016 genomes AAV. A human dosage may be about 1×1013 genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a CRISPR enzyme, operably linked to said promoter; (iii) a selectable marker; (iv) an origin of replication; and (v) a transcription terminator downstream of and operably linked to (ii). The plasmid can also encode the RNA components of a CRISPR complex, but one or more of these may instead be encoded on a different vector.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

The dosage used for the compositions provided herein include dosages for repeated administration or repeat dosing. In particular embodiments, the administration is repeated within a period of several weeks, months, or years. Suitable assays can be performed to obtain an optimal dosage regime. Repeated administration can allow the use of lower dosage, which can positively affect off-target modifications.

RNA Delivery

In particular embodiments, RNA based delivery is used. In these embodiments, mRNA of the CRISPR effector protein is delivered together with in vitro transcribed guide RNA. Liang et al. describes efficient genome editing using RNA based delivery (Protein Cell. 2015 May; 6(5): 363-372).

RNA delivery: The CRISPR enzyme, for instance a Cpf1, and/or any of the present RNAs, for instance a guide RNA, can also be delivered in the form of RNA. Cpf1 mRNA can be generated using in vitro transcription. For example, Cpf1 mRNA can be synthesized using a PCR cassette containing the following elements: T7_promoter-kozak sequence (GCCACC)-Cpf1-3′ UTR from beta globin-polyA tail (a string of 120 or more adenines). The cassette can be used for transcription by T7 polymerase. Guide RNAs can also be transcribed using in vitro transcription from a cassette containing T7_promoter-GG-guide RNA sequence.

To enhance expression and reduce possible toxicity, the CRISPR enzyme-coding sequence and/or the guide RNA can be modified to include one or more modified nucleoside e.g. using pseudo-U or 5-Methyl-C.

mRNA delivery methods are especially promising for liver delivery currently.

Much clinical work on RNA delivery has focused on RNAi or antisense, but these systems can be adapted for delivery of RNA for implementing the present invention. References below to RNAi etc. should be read accordingly.

CRISPR enzyme mRNA and guide RNA might also be delivered separately. CRISPR enzyme mRNA can be delivered prior to the guide RNA to give time for CRISPR enzyme to be expressed. CRISPR enzyme mRNA might be administered 1-12 hours (preferably around 2-6 hours) prior to the administration of guide RNA.

Alternatively, CRISPR enzyme mRNA and guide RNA can be administered together. Advantageously, a second booster dose of guide RNA can be administered 1-12 hours (preferably around 2-6 hours) after the initial administration of CRISPR enzyme mRNA+guide RNA.

RNP

In particular embodiments, pre-complexed guide RNA and CRISPR effector protein are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

Indeed, RNA delivery is a useful method of in vivo delivery. It is possible to deliver Cpf1 and gRNA (and, for instance, HR repair template) into cells using liposomes or particles. Thus delivery of the CRISPR enzyme, such as a Cpf1 and/or delivery of the RNAs of the invention may be in RNA form and via microvesicles, liposomes or particles. For example, Cpf1 mRNA and gRNA can be packaged into liposomal particles for delivery in vivo. Liposomal transfection reagents such as lipofectamine from Life Technologies and other reagents on the market can effectively deliver RNA molecules into the liver.

Means of delivery of RNA also preferred include delivery of RNA via nanoparticles (Cho, S., Goldberg, M., Son, S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder, A., Levins, C., Cortez, C., Langer, R., and Anderson, D., Lipid-based nanotherapeutics for siRNA delivery, Journal of Internal Medicine, 267: 9-21, 2010, PMID: 20059641). Indeed, exosomes have been shown to be particularly useful in delivery siRNA, a system with some parallels to the CRISPR system. For instance, El-Andaloussi S, et al. (“Exosome-mediated delivery of siRNA in vitro and in vivo.” Nat Protoc. 2012 December; 7(12):2112-26. doi: 10.1038/nprot.2012.131. Epub 2012 Nov. 15.) describe how exosomes are promising tools for drug delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purify and characterized from transfected cell supernatant, then RNA is loaded into the exosomes. Delivery or administration according to the invention can be performed with exosomes, in particular but not limited to the brain. Vitamin E (a-tocopherol) may be conjugated with CRISPR Cas and delivered to the brain along with high density lipoprotein (HDL), for example in a similar manner as was done by Uno et al. (HUMAN GENE THERAPY 22:711-719 (June 2011)) for delivering short-interfering RNA (siRNA) to the brain. Mice were infused via Osmotic minipumps (model 1007D; Alzet, Cupertino, Calif.) filled with phosphate-buffered saline (PBS) or free TocsiBACE or Toc-siBACE/HDL and connected with Brain Infusion Kit 3 (Alzet). A brain-infusion cannula was placed about 0.5 mm posterior to the bregma at midline for infusion into the dorsal third ventricle. Uno et al. found that as little as 3 nmol of Toc-siRNA with HDL could induce a target reduction in comparable degree by the same ICV infusion method. A similar dosage of CRISPR Cas conjugated to a-tocopherol and co-administered with HDL targeted to the brain may be contemplated for humans in the present invention, for example, about 3 nmol to about 3 μmol of CRISPR Cas targeted to the brain may be contemplated.

Zou et al. ((HUMAN GENE THERAPY 22:465-475 (April 2011)) describes a method of lentiviral-mediated delivery of short-hairpin RNAs targeting PKCγ for in vivo gene silencing in the spinal cord of rats. Zou et al. administered about 10 l of a recombinant lentivirus having a titer of 1×109 transducing units (TU)/ml by an intrathecal catheter. A similar dosage of CRISPR Cas expressed in a lentiviral vector may be contemplated for humans in the present invention, for example, about 10-50 ml of CRISPR Cas in a lentivirus having a titer of 1×109 transducing units (TU)/ml may be contemplated. A similar dosage of CRISPR Cas expressed in a lentiviral vector targeted to the brain may be contemplated for humans in the present invention, for example, about 10-50 ml of CRISPR Cas targeted to the brain in a lentivirus having a titer of 1×109 transducing units (TU)/ml may be contemplated.

Anderson et al. (US 20170079916) provides a modified dendrimer nanoparticle for the delivery of therapeutic, prophylactic and/or diagnostic agents to a subject, comprising: one or more zero to seven generation alkylated dendrimers; one or more amphiphilic polymers; and one or more therapeutic, prophylactic and/or diagnostic agents encapsulated therein. One alkylated dendrimer may be selected from the group consisting of poly(ethyleneimine), poly(polyproylenimine), diaminobutane amine polypropylenimine tetramine and poly(amido amine). The therapeutic, prophylactic and diagnostic agent may be selected from the group consisting of proteins, peptides, carbohydrates, nucleic acids, lipids, small molecules and combinations thereof.

Anderson et al. (US 20160367686) provides a compound of Formula (I):

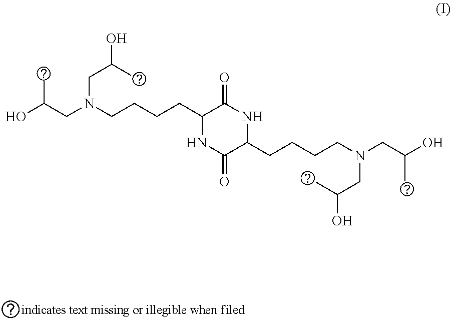 [SMILES: CC(O)CN(CCCCC1NC(=O)C(CCCCN(CC(C)O)CC(C)O)NC1=O)CC(C)O]

and salts thereof, wherein each instance of R L is independently optionally substituted C6-C40 alkenyl, and a composition for the delivery of an agent to a subject or cell comprising the compound, or a salt thereof; an agent; and optionally, an excipient. The agent may be an organic molecule, inorganic molecule, nucleic acid, protein, peptide, polynucleotide, targeting agent, an isotopically labeled chemical compound, vaccine, an immunological agent, or an agent useful in bioprocessing. The composition may further comprise cholesterol, a PEGylated lipid, a phospholipid, or an apolipoprotein.

Anderson et al. (US20150232883) provides a delivery particle formulations and/or systems, preferably nanoparticle delivery formulations and/or systems, comprising (a) a CRISPR-Cas system RNA polynucleotide sequence; or (b) Cas9; or (c) both a CRISPR-Cas system RNA polynucleotide sequence and Cas9; or (d) one or more vectors that contain nucleic acid molecule(s) encoding (a), (b) or (c), wherein the CRISPR-Cas system RNA polynucleotide sequence and the Cas9 do not naturally occur together. The delivery particle formulations may further comprise a surfactant, lipid or protein, wherein the surfactant may comprise a cationic lipid.

Anderson et al. (US20050123596) provides examples of microparticles that are designed to release their payload when exposed to acidic conditions, wherein the microparticles comprise at least one agent to be delivered, a pH triggering agent, and a polymer, wherein the polymer is selected from the group of polymethacrylates and polyacrylates.

Anderson et al (US 20020150626) provides lipid-protein-sugar particles for delivery of nucleic acids, wherein the polynucleotide is encapsulated in a lipid-protein-sugar matrix by contacting the polynucleotide with a lipid, a protein, and a sugar; and spray drying mixture of the polynucleotide, the lipid, the protein, and the sugar to make microparticles.

In terms of local delivery to the brain, this can be achieved in various ways. For instance, material can be delivered intrastriatally e.g. by injection. Injection can be performed stereotactically via a craniotomy.

Enhancing NHEJ or HR efficiency is also helpful for delivery. It is preferred that NHEJ efficiency is enhanced by co-expressing end-processing enzymes such as Trex2 (Dumitrache et al. Genetics. 2011 August; 188(4): 787-797). It is preferred that HR efficiency is increased by transiently inhibiting NHEJ machineries such as Ku70 and Ku86. HR efficiency can also be increased by co-expressing prokaryotic or eukaryotic homologous recombination enzymes such as RecBCD, RecA.

Particles

In some aspects or embodiments, a composition comprising a delivery particle formulation may be used. In some aspects or embodiments, the formulation comprises a CRISPR complex, the complex comprising a CRISPR protein and-a guide which directs sequence-specific binding of the CRISPR complex to a target sequence. In some embodiments, the delivery particle comprises a lipid-based particle, optionally a lipid nanoparticle, or cationic lipid and optionally biodegradable polymer. In some embodiments, the cationic lipid comprises 1,2-dioleoyl-3-trimethylammonium-propane (DOTAP). In some embodiments, the hydrophilic polymer comprises ethylene glycol or polyethylene glycol. In some embodiments, the delivery particle further comprises a lipoprotein, preferably cholesterol. In some embodiments, the delivery particles are less than 500 nm in diameter, optionally less than 250 nm in diameter, optionally less than 100 nm in diameter, optionally about 35 nm to about 60 nm in diameter.

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

In terms of this invention, it is preferred to have one or more components of CRISPR complex, e.g., CRISPR enzyme or mRNA or guide RNA delivered using nanoparticles or lipid envelopes. Other delivery systems or vectors are may be used in conjunction with the nanoparticle aspects of the invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate.

It will be understood that the size of the particle will differ depending as to whether it is measured before or after loading. Accordingly, in particular embodiments, the term “nanoparticles” may apply only to the particles pre loading.

Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, concerning particles, methods of making and using them and measurements thereof.

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

CRISPR enzyme mRNA and guide RNA may be delivered simultaneously using particles or lipid envelopes; for instance, CRISPR enzyme and RNA of the invention, e.g., as a complex, can be delivered via a particle as in Dahlman et al., WO2015089419 A2 and documents cited therein, such as 7C1 (see, e.g., James E. Dahlman and Carmen Barnes et al. Nature Nanotechnology (2014) published online 11 May 2014, doi: 10. 1038/nnano.2014.84), e.g., delivery particle comprising lipid or lipidoid and hydrophilic polymer, e.g., cationic lipid and hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5), wherein particles are formed using an efficient, multistep process wherein first, effector protein and RNA are mixed together, e.g., at a 1:1 molar ratio, e.g., at room temperature, e.g., for 30 minutes, e.g., in sterile, nuclease free 1×PBS; and separately, DOTAP, DMPC, PEG, and cholesterol as applicable for the formulation are dissolved in alcohol, e.g., 100% ethanol; and, the two solutions are mixed together to form particles containing the complexes).

Nucleic acid-targeting effector proteins (such as a Type V protein such Cpf1) mRNA and guide RNA may be delivered simultaneously using particles or lipid envelopes. Examples of suitable particles include but are not limited to those described in U.S. Pat. No. 9,301,923.

For example, Su X, Fricke J, Kavanagh D G, Irvine D J (“In vitro and in vivo mRNA delivery using lipid-enveloped pH-responsive polymer nanoparticles” Mol Pharm. 2011 Jun. 6; 8(3):774-87. doi: 10.1021/mpl00390w. Epub 2011 Apr. 1) describes biodegradable core-shell structured nanoparticles with a poly(P3-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

Liu et al. (US 20110212179) provides bimodal porous polymer microspheres comprising a base polymer, wherein the particle comprises macropores having a diameter ranging from about 20 to about 500 microns and micropores having a diameter ranging from about 1 to about 70 microns, and wherein the microspheres have a diameter ranging from about 50 to about 1100 microns.

Berg et al. (US20160174546) a nanolipid delivery system, in particular a nano-particle concentrate, comprising: a composition comprising a lipid, oil or solvent, the composition having a viscosity of less than 100 cP at 25.degree. C. and a Kauri Butanol solvency of greater than 25 Kb; and at least one amphipathic compound selected from the group consisting of an alkoxylated lipid, an alkoxylated fatty acid, an alkoxylated alcohol, a heteroatomic hydrophilic lipid, a heteroatomic hydrophilic fatty acid, a heteroatomic hydrophilic alcohol, a diluent, and combinations thereof, wherein the compound is derived from a starting compound having a viscosity of less than 1000 cP at 50.degree. C., wherein the concentrate is configured to provide a stable nano emulsion having a D50 and a mean average particle size distribution of less than 100 nm when diluted.

Liu et al. (US 20140301951) provides a protocell nanostructure comprising: a porous particle core comprising a plurality of pores; and at least one lipid bilayer surrounding the porous particle core to form a protocell, wherein the protocell is capable of loading one or more cargo components to the plurality of pores of the porous particle core and releasing the one or more cargo components from the porous particle core across the surrounding lipid bilayer.

Chromy et al. (US 20150105538) provides methods and systems for assembling, solubilizing and/or purifying a membrane associated protein in a nanolipoprotein particle, which comprise a temperature transition cycle performed in presence of a detergent, wherein during the temperature transition cycle the nanolipoprotein components are brought to a temperature above and below the gel to liquid crystalling transition temperature of the membrane forming lipid of the nanolipoprotein particle.

Bader et al. (US 20150250725), provides a method for producing a lipid particle comprising the following: i) providing a first solution comprising denatured apolipoprotein, ii) adding the first solution to a second solution comprising at least two lipids and a detergent but no apolipoprotein, and iii) removing the detergent from the solution obtained in ii) and thereby producing a lipid particle.

Mirkin et al., (US20100129793) provides a method of preparing a composite particle comprising the steps of (a) admixing a dielectric component and a magnetic component to form a first intermediate, (b) admixing the first intermediate and gold seeds to form a second intermediate, and (c) forming a gold shell on the second intermediate by admixing the second intermediate with a gold source and a reducing agent to form said composite particle.

In one embodiment, particles/nanoparticles based on self assembling bioadhesive polymers are contemplated, which may be applied to oral delivery of peptides, intravenous delivery of peptides and nasal delivery of peptides, all to the brain. Other embodiments, such as oral absorption and ocular delivery of hydrophobic drugs are also contemplated. The molecular envelope technology involves an engineered polymer envelope which is protected and delivered to the site of the disease (see, e.g., Mazza, M. et al. ACSNano, 2013. 7(2): 1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Pharm, 2012. 9(6):1764-74; Garrett, N. L., et al. J Biophotonics, 2012. 5(5-6):458-68; Garrett, N. L., et al. J Raman Spect, 2012. 43(5):681-688; Ahmad, S., et al. J Royal Soc Interface 2010. 7:S423-33; Uchegbu, I. F. Expert Opin Drug Deliv, 2006. 3(5):629-40; Qu, X.,et al. Biomacromolecules, 2006. 7(12):3452-9 and Uchegbu, I. F., et al. Int J Pharm, 2001. 224:185-199). Doses of about 5 mg/kg are contemplated, with single or multiple doses, depending on the target tissue.

In one embodiment, particles/nanoparticles that can deliver RNA to a cancer cell to stop tumor growth developed by Dan Anderson's lab at MIT may be used/and or adapted to the CRISPR Cas system of the present invention. In particular, the Anderson lab developed fully automated, combinatorial systems for the synthesis, purification, characterization, and formulation of new biomaterials and nanoformulations. See, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3): 1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93.

The lipid particles developed by the Qiaobing Xu's lab at Tufts University may be used/adapted to the present delivery system for cancer therapy. See Wang et al., J. Control Release, 2017 Jan. 31. pii: S0168-3659(17)30038-X. doi: 10.1016/j.jconrel.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, Sep. 2014; and Wang et al., Agnew Chem Int Ed Engl., 53(11):2893-8, Mar. 10, 2014.

US patent application 20110293703 relates to lipidoid compounds are also particularly useful in the administration of polynucleotides, which may be applied to deliver the CRISPR Cas system of the present invention. In one aspect, the aminoalcohol lipidoid compounds are combined with an agent to be delivered to a cell or a subject to form microparticles, nanoparticles, liposomes, or micelles. The agent to be delivered by the particles, liposomes, or micelles may be in the form of a gas, liquid, or solid, and the agent may be a polynucleotide, protein, peptide, or small molecule. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Publication No. 20110293703 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30−100° C., preferably at approximately 50-90° C. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or other alkylating agent, and/or they may be acylated.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to the CRISPR Cas system of the present invention.

In another embodiment, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to the CRISPR Cas system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

Zhu et al. (US20140348900) provides for a process for preparing liposomes, lipid discs, and other lipid nanoparticles using a multi-port manifold, wherein the lipid solution stream, containing an organic solvent, is mixed with two or more streams of aqueous solution (e.g., buffer). In some aspects, at least some of the streams of the lipid and aqueous solutions are not directly opposite of each other. Thus, the process does not require dilution of the organic solvent as an additional step. In some embodiments, one of the solutions may also contain an active pharmaceutical ingredient (API). This invention provides a robust process of liposome manufacturing with different lipid formulations and different payloads. Particle size, morphology, and the manufacturing scale can be controlled by altering the port size and number of the manifold ports, and by selecting the flow rate or flow velocity of the lipid and aqueous solutions.

Cullis et al. (US 20140328759) provides limit size lipid nanoparticles with a diameter from 10-100 nm, in particular comprising a lipid bilayer surrounding an aqueous core. Methods and apparatus for preparing such limit size lipid nanoparticles are also disclosed.

Manoharan et al. (US 20140308304) provides cationic lipids of formula (I)

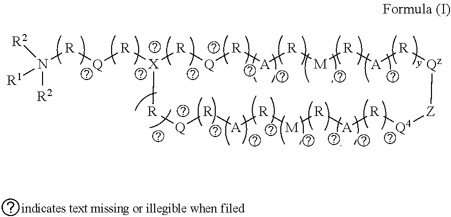

or a salt thereof, wherein X is N or P; R′ is absent, hydrogen, or alkyl; with respect to R1 and R2, (i) R1 and R2 are each, independently, optionally substituted alkyl, alkenyl, alkynyl, cycloalkyl, cycloalkylalkyl, heterocycle or R10; (ii) R1 and R2, together with the nitrogen atom to which they are attached, form an optionally substituted heterocylic ring; or (iii) one of R1 and R2 is optionally substituted alkyl, alkenyl, alkynyl, cycloalkyl, cycloalkylalkyl, or heterocycle, and the other forms a 4-10 member heterocyclic ring or heteroaryl with (a) the adjacent nitrogen atom and (b) the (R) a group adjacent to the nitrogen atom; each occurrence of R is, independently, —(CR3R4)-; each occurrence of R3 and R4 are, independently H, halogen, OH, alkyl, alkoxy, —NH.sub.2, alkylamino, or dialkylamino; or R3 and R4, together with the carbon atom to which they are directly attached, form a cycloalkyl group, wherein no more than three R groups in each chain attached to the atom X* are cycloalkyl; each occurrence of R. sup. 10 is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R10 groups; Q is absent or is —O—, —NH—, —S—, —C(O)O—, —OC(O)—, —C(O)N(R4)-, —N(R5)C(O)—, —S—S—, —OC(O)O—, —O—N.dbd.C(R5)-, —C(R5).dbd.N—O—, —OC(O)N(R5)-, —N(R5)C(O)N(R5)-, —N(R5)C(O)O—, —C(O)S—, —C(S)O— or —C(R5).dbd.N—O—C(O)—; Q1 and Q2 are each, independently, absent, —O—, —S—, —OC(O)—, —C(O)O—, —SC(O)—, —C(O)S—, —OC(S)—, —C(S)O—, —S—S—, —C(O)(NR5)-, —N(R5)C(O)—, —C(S)(NR5)-, —N(R5)C(O)—, —N(R5)C(O)N(R5)-, or —OC(O)O—; Q3 and Q4 are each, independently, H, —(CR3R4)-, aryl, or a cholesterol moiety; each occurrence of A1, A2, A3 and A4 is, independently, —(CR5R5-CR5.dbd.CR5)-; each occurrence of R5 is, independently, H or alkyl; M1 and M2 are each, independently, a biodegradable group (e.g., —OC(O)—, —C(O)O—, —SC(O)—, —C(O)S—, —OC(S)—, —C(S)O—, —S—S—, —C(R5).dbd.N—, —N.dbd.C(R5)-, —C(R5).dbd.N—O—, —O—N.dbd.C(R5)-, —C(O)(NR5)-, —N(R5)C(O)—, —C(S)(NR5)-, —N(R5)C(O)—, —N(R5)C(O)N(R5)-, —OC(O)O—, —OSi(R5).sub.2O—, —C(O)(CR3R4)C(O)O—, or —OC(O)(CR3R4)C(O)—); Z is absent, alkylene or —O—P(O)(OH)—O—; each ------ attached to Z is an optional bond, such that when Z is absent, Q3 and Q4 are not directly covalently bound together; a is 1, 2, 3, 4, 5 or 6; b is 0, 1, 2, or 3; c, d, e, f, i, j, m, n, q and r are each, independently, 0, 1, 2, 3, 4, 5, 6, 7, 8, 9, or 10; g and h are each, independently, 0, 1 or 2; k and 1 are each, independently, 0 or 1, where at least one of k and 1 is 1; and o and p are each, independently, 0, 1 or 2, wherein Q3 and Q4 are each, independently, separated from the tertiary atom marked with an asterisk (X*) by a chain of 8 or more atoms. The cationic lipid can be used with other lipid components such as cholesterol and PEG-lipids to form lipid nanoparticles with oligonucleotides, to facilitate the cellular uptake and endosomal escape, and to knockdown target mRNA both in vitro and in vivo.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP or CRISPR-Cas RNA in or associated with the LNP may be contemplated, especially for a formulation containing DLinKC2-DMA.

Preparation of LNPs and CRISPR Cas encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(co-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprised of cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Nanoparticle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be −70 nm in diameter. RNA encapsulation efficiency may be determined by removal of free RNA using VivaPureD MiniH columns (Sartorius Stedim Biotech) from samples collected before and after dialysis. The encapsulated RNA may be extracted from the eluted nanoparticles and quantified at 260 nm. RNA to lipid ratio was determined by measurement of cholesterol content in vesicles using the Cholesterol E enzymatic assay from Wako Chemicals USA (Richmond, Va.). In conjunction with the herein discussion of LNPs and PEG lipids, PEGylated liposomes or LNPs are likewise suitable for delivery of a CRISPR-Cas system or components thereof.

Preparation of large LNPs may be used/and or adapted from Rosin et al, Molecular Therapy, vol. 19, no. 12, pages 1286-2200, December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/l, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano Z S, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an RNA to total lipid ratio of approximately 1:10 (wt:wt), followed by incubation for 30 minutes at 37° C. to form loaded LNPs. The mixture may be subsequently dialyzed overnight in PBS and filtered with a 0.45-am syringe filter.

Preassembled recombinant CRISPR-Cpf1 complexes comprising Cpf1 and crRNA may be transfected, for example by electroporation, resulting in high mutation rates and absence of detectable off-target mutations. Hur, J. K. et al, Targeted mutagenesis in mice by electroporation of Cpf1 ribonucleoproteins, Nat Biotechnol. 2016 Jun. 6. doi: 10.1038/nbt.3596. [Epub ahead of print]

In terms of local delivery to the brain, this can be achieved in various ways. For instance, material can be delivered intrastriatally e.g. by injection. Injection can be performed stereotactically via a craniotomy.

Enhancing NHEJ or HR efficiency is also helpful for delivery. It is preferred that NHEJ efficiency is enhanced by co-expressing end-processing enzymes such as Trex2 (Dumitrache et al. Genetics. 2011 August; 188(4): 787-797). It is preferred that HR efficiency is increased by transiently inhibiting NHEJ machineries such as Ku70 and Ku86. HR efficiency can also be increased by co-expressing prokaryotic or eukaryotic homologous recombination enzymes such as RecBCD, RecA.

In some embodiments, sugar-based particles may be used, for example GalNAc, as described herein and with reference to WO2014118272 (incorporated herein by reference) and Nair, J K et al., 2014, Journal of the American Chemical Society 136 (49), 16958-16961) and the teaching herein, especially in respect of delivery applies to all particles unless otherwise apparent. This may be considered to be a sugar-based particle and further details on other particle delivery systems and/or formulations are provided herein. GalNAc can therefore be considered to be a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt. ˜2000) activated as PFP (pentafluorophenyl) esters onto 5′-hexylamino modified oligonucleotides (5′-HA ASOs, mol. wt. ˜8000 Da; Ostergaard et al., Bioconjugate Chem., 2015, 26 (8), pp 1451-1455). Similarly, poly(acrylate) polymers have been described for in vivo nucleic acid delivery (see WO2013158141 incorporated herein by reference). In further alternative embodiments, pre-mixing CRISPR nanoparticles (or protein complexes) with naturally occurring serum proteins may be used in order to improve delivery (Akinc A et al, 2010, Molecular Therapy vol. 18 no. 7, 1357-1364).

Nanoclews

Further, the programmable nucleic acid modifying agents may be delivered using nanoclews, for example as described in Sun W et al, Cocoon-like self-degradable DNA nanoclew for anticancer drug delivery., J Am Chem Soc. 2014 Oct. 22; 136(42):14722-5. doi: 10.1021/ja5088024. Epub 2014 Oct. 13. ; or in Sun W et al, Self-Assembled DNA Nanoclews for the Efficient Delivery of CRISPR-Cas9 for Genome Editing., Angew Chem Int Ed Engl. 2015 Oct. 5; 54(41):12029-33. doi: 10.1002/anie.201506030. Epub 2015 Aug. 27.

LNP

In some embodiments, delivery is by encapsulation of the programmable nucleic acid modifying agents in a lipid particle such as an LNP. In some embodiments, therefore, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to the CRISPR Cas system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP or CRISPR-Cas RNA in or associated with the LNP may be contemplated, especially for a formulation containing DLinKC2-DMA.

Preparation of LNPs and CRISPR Cas encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(co-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). When required, 0.2% SP-DiOC18 (Invitrogen, Burlington, Canada) may be incorporated to assess cellular uptake, intracellular delivery, and biodistribution. Encapsulation may be performed by dissolving lipid mixtures comprised of cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/l citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Nanoparticle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be −70 nm in diameter. RNA encapsulation efficiency may be determined by removal of free RNA using VivaPureD MiniH columns (Sartorius Stedim Biotech) from samples collected before and after dialysis. The encapsulated RNA may be extracted from the eluted nanoparticles and quantified at 260 nm. RNA to lipid ratio was determined by measurement of cholesterol content in vesicles using the Cholesterol E enzymatic assay from Wako Chemicals USA (Richmond, Va.). In conjunction with the herein discussion of LNPs and PEG lipids, PEGylated liposomes or LNPs are likewise suitable for delivery of a CRISPR-Cas system or components thereof.

A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/l, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano Z S, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an RNA to total lipid ratio of approximately 1:10 (wt:wt), followed by incubation for 30 minutes at 37° C. to form loaded LNPs. The mixture may be subsequently dialyzed overnight in PBS and filtered with a 0.45-am syringe filter.

Spherical Nucleic Acid (SNA™) constructs and other nanoparticles (particularly gold nanoparticles) are also contemplated as a means to delivery CRISPR-Cas system to intended targets. Significant data show that AuraSense Therapeutics' Spherical Nucleic Acid (SNA™) constructs, based upon nucleic acid-functionalized gold nanoparticles, are useful.

Literature that may be employed in conjunction with herein teachings include: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG). This system has been used, for example, as a means to target tumor neovasculature expressing integrins and deliver siRNA inhibiting vascular endothelial growth factor receptor-2 (VEGF R2) expression and thereby achieve tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling nanoparticles of Schiffelers et al.

The nanoplexes of Bartlett et al. (PNAS, Sep. 25, 2007,vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA nanoparticles may be formed by using cyclodextrin-containing polycations. Typically, nanoparticles were formed in water at a charge ratio of 3 (+/−) and an siRNA concentration of 0.5 g/liter. One percent of the adamantane-PEG molecules on the surface of the targeted nanoparticles were modified with Tf (adamantane-PEG-Tf). The nanoparticles were suspended in a 5% (wt/vol) glucose carrier solution for injection.

Davis et al. (Nature, Vol 464, 15 Apr. 2010) conducts a RNA clinical trial that uses a targeted nanoparticle-delivery system (clinical trial registration number NCT00689065). Patients with solid cancers refractory to standard-of-care therapies are administered doses of targeted nanoparticles on days 1, 3, 8 and 10 of a 21-day cycle by a 30-min intravenous infusion. The nanoparticles consist of a synthetic delivery system containing: (1) a linear, cyclodextrin-based polymer (CDP), (2) a human transferrin protein (TF) targeting ligand displayed on the exterior of the nanoparticle to engage TF receptors (TFR) on the surface of the cancer cells, (3) a hydrophilic polymer (polyethylene glycol (PEG) used to promote nanoparticle stability in biological fluids), and (4) siRNA designed to reduce the expression of the RRM2 (sequence used in the clinic was previously denoted siR2B+5). The TFR has long been known to be upregulated in malignant cells, and RRM2 is an established anti-cancer target. These nanoparticles (clinical version denoted as CALAA-01) have been shown to be well tolerated in multi-dosing studies in non-human primates. Although a single patient with chronic myeloid leukaemia has been administered siRNA by liposomal delivery, Davis et al.'s clinical trial is the initial human trial to systemically deliver siRNA with a targeted delivery system and to treat patients with solid cancer. To ascertain whether the targeted delivery system can provide effective delivery of functional siRNA to human tumors, Davis et al. investigated biopsies from three patients from three different dosing cohorts; patients A, B and C, all of whom had metastatic melanoma and received CALAA-01 doses of 18, 24 and 30 mg m−2 siRNA, respectively. Similar doses may also be contemplated for the CRISPR Cas system of the present invention. The delivery of the invention may be achieved with nanoparticles containing a linear, cyclodextrin-based polymer (CDP), a human transferrin protein (TF) targeting ligand displayed on the exterior of the nanoparticle to engage TF receptors (TFR) on the surface of the cancer cells and/or a hydrophilic polymer (for example, polyethylene glycol (PEG) used to promote nanoparticle stability in biological fluids).

U.S. Pat. No. 8,709,843, incorporated herein by reference, provides a drug delivery system for targeted delivery of therapeutic agent-containing particles to tissues, cells, and intracellular compartments. The invention provides targeted particles comprising comprising polymer conjugated to a surfactant, hydrophilic polymer or lipid.

U.S. Pat. No. 6,007,845, incorporated herein by reference, provides particles which have a core of a multiblock copolymer formed by covalently linking a multifunctional compound with one or more hydrophobic polymers and one or more hydrophilic polymers, and conatin a biologically active material.

U.S. Pat. No. 5,855,913, incorporated herein by reference, provides a particulate composition having aerodynamically light particles having a tap density of less than 0.4 g/cm3 with a mean diameter of between 5.im and 30 i m, incorporating a surfactant on the surface thereof for drug delivery to the pulmonary system.

U.S. Pat. No. 5,985,309, incorporated herein by reference, provides particles incorporating a surfactant and/or a hydrophilic or hydrophobic complex of a positively or negatively charged therapeutic or diagnostic agent and a charged molecule of opposite charge for delivery to the pulmonary system.

U.S. Pat. No. 5,543,158, incorporated herein by reference, provides biodegradable injectable particles having a biodegradable solid core containing a biologically active material and poly(alkylene glycol) moieties on the surface.

WO2012135025 (also published as US20120251560), incorporated herein by reference, describes conjugated polyethyleneimine (PEI) polymers and conjugated aza-macrocycles (collectively referred to as “conjugated lipomer” or “lipomers”). In certain embodiments, it can envisioned that such conjugated lipomers can be used in the context of the CRISPR-Cas system to achieve in vitro, ex vivo and in vivo genomic perturbations to modify gene expression, including modulation of protein expression.

In one embodiment, the nanoparticle may be epoxide-modified lipid-polymer, advantageously 7C1 (see, e.g., James E. Dahlman and Carmen Barnes et al. Nature Nanotechnology (2014) published online 11 May 2014, doi:10.1038/nnano.2014.84). C71 was synthesized by reacting C15 epoxide-terminated lipids with PEI600 at a 14:1 molar ratio, and was formulated with C14PEG2000 to produce nanoparticles (diameter between 35 and 60 nm) that were stable in PBS solution for at least 40 days.

An epoxide-modified lipid-polymer may be utilized to deliver the CRISPR-Cas system of the present invention to pulmonary, cardiovascular or renal cells, however, one of skill in the art may adapt the system to deliver to other target organs. Dosage ranging from about 0.05 to about 0.6 mg/kg are envisioned. Dosages over several days or weeks are also envisioned, with a total dosage of about 2 mg/kg.

In some embodiments, the LNP for deliverting the RNA molecules is prepared by methods known in the art, such as those described in, for example, WO 2005/105152 (PCT/EP2005/004920), WO 2006/069782 (PCT/EP2005/014074), WO 2007/121947 (PCT/EP2007/003496), and WO 2015/082080 (PCT/EP2014/003274), which are herein incorporated by reference. LNPs aimed specifically at the enhanced and improved delivery of siRNA into mammalian cells are described in, for example, Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014), which are herein incorporated by reference and may be applied to the present technology.

In some embodiments, the LNP includes any LNP disclosed in WO 2005/105152 (PCT/EP2005/004920), WO 2006/069782 (PCT/EP2005/014074), WO 2007/121947 (PCT/EP2007/003496), and WO 2015/082080 (PCT/EP2014/003274).

In some embodiments, the LNP includes at least one lipid having Formula I:

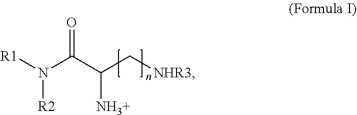

wherein R1 and R2 are each and independently selected from the group comprising alkyl, n is any integer between 1 and 4, and R3 is an acyl selected from the group comprising lysyl, ornithyl, 2,4-diaminobutyryl, histidyl and an acyl moiety according to Formula II:

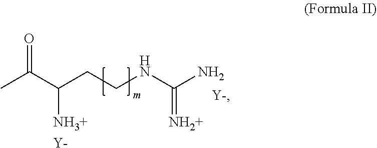

wherein m is any integer from 1 to 3 and Y- is a pharmaceutically acceptable anion. In some embodiments, a lipid according to Formula I includes at least two asymmetric C atoms. In some embodiments, enantiomers of Formula I include, but are not limited to, R-R; S-S; R—S and S-R enantiomer.

In some embodiments, R1 is lauryl and R2 is myristyl. In another embodiment, R1 is palmityl and R2 is oleyl. In some embodiments, m is 1 or 2. In some embodiments, Y- is selected from halogenids, acetate or trifluoroacetate.

In some embodiments, the LNP comprises one or more lipids select from:
arginyl-2,3-diamino propionic acid-N-palmityl-N-oleyl-amide trihydrochloride (Formula III):

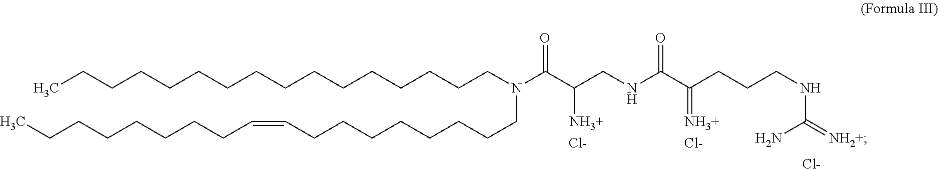

arginyl-2,3-diamino propionic acid-N-lauryl-N-myristylamide trihydrochloride (Formula IV):

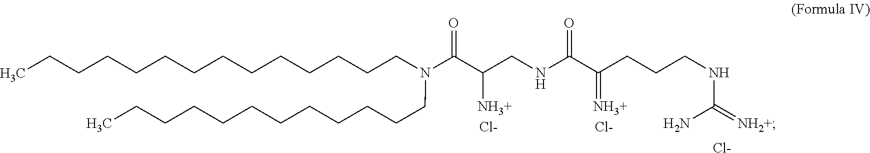

and
ε-arginyl-lysine-N-lauryl-N-myristyl-amide trihydrochloride (Formula V):

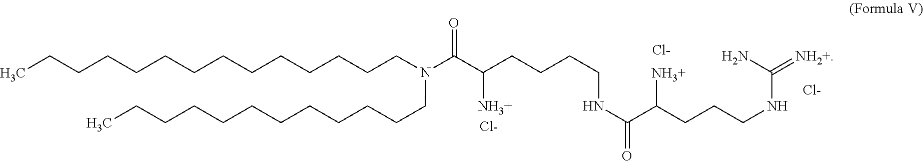

In some embodiments, the LNP also includes a constituent. By way of example, but not by way of limitation, in some embodiments, the constituent is selected from peptides, proteins, oligonucleotides, polynucleotides, nucleic acids, or a combination thereof. In some embodiments, the constituent is an antibody, e.g., a monoclonal antibody. In some embodiments, the constituent is a nucleic acid selected from, e.g., ribozymes, aptamers, spiegelmers, DNA, RNA, PNA, LNA, or a combination thereof. In some embodiments, the nucleic acid is gRNA and/or mRNA.

In some embodiments, the constituent of the LNP comprises an mRNA encoding a CRIPSR effector protein. In some embodiments, the constituent of the LNP comprises an mRNA encoding a Type-II, Type-V, or Type-VI CRIPSR effector protein. In some embodiments, the constituent of the LNP comprises an mRNA encoding an RNA-guided DNA binding protein. In some embodiments, the constituent of the LNP comprises an mRNA encoding an RNA-guided RNA binding protein.

In some embodiments, the constituent of the LNP further comprises one or more guide RNA. In some embodiments, the LNP is configured to deliver the aforementioned mRNA and guide RNA to vascular endothelium. In some embodiments, the LNP is configured to deliver the aforementioned mRNA and guide RNA to pulmonary endothelium. In some embodiments, the LNP is configured to deliver the aforementioned mRNA and guide RNA to liver. In some embodiments, the LNP is configured to deliver the aforementioned mRNA and guide RNA to lung. In some embodiments, the LNP is configured to deliver the aforementioned mRNA and guide RNA to hearts. In some embodiments, the LNP is configured to deliver the aforementioned mRNA and guide RNA to spleen. In some embodiments, the LNP is configured to deliver the aforementioned mRNA and guide RNA to kidney. In some embodiments, the LNP is configured to deliver the aforementioned mRNA and guide RNA to pancrea. In some embodiments, the LNP is configured to deliver the aforementioned mRNA and guide RNA to brain. In some embodiments, the LNP is configured to deliver the aforementioned mRNA and guide RNA to macrophages.

In some embodiments, the LNP also includes at least one helper lipid. In some embodiments, the helper lipid is selected from phospholipids and steroids. In some embodiments, the phospholipids are di- and/or monoester of the phosphoric acid. In some embodiments, the phospholipids are phosphoglycerides and/or sphingolipids. In some embodiments, the steroids are naturally occurring and/or synthetic compounds based on the partially hydrogenated cyclopenta[a]phenanthrene. In some embodiments, the steroids contain 21 to 30 C atoms. In some embodiments, the steroid is cholesterol. In some embodiments, the helper lipid is selected from 1,2-diphytanoyl-sn-glycero-3-phosphoethanolamine (DPhyPE), ceramide, and 1,2-dioleylsn-glycero-3-phosphoethanolamine (DOPE).

In some embodiments, the at least one helper lipid comprises a moiety selected from the group comprising a PEG moiety, a HEG moiety, a polyhydroxyethyl starch (polyHES) moiety and a polypropylene moiety. In some embodiments, the moiety has a molecule weight between about 500 to 10,000 Da or between about 2,000 to 5,000 Da. In some embodiments, the PEG moiety is selected from 1,2-distearoyl-sn-glycero-3 phosphoethanolamine, 1,2-dialkyl-sn-glycero-3-phosphoethanolamine, and Ceramide-PEG. In some embodiments, the PEG moiety has a molecular weight between about 500 to 10,000 Da or between about 2,000 to 5,000 Da. In some embodiments, the PEG moiety has a molecular weight of 2,000 Da.

In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

In some embodiments, the LNP includes any of −3-arginyl-2,3-diaminopropionic acid-N-palmityl-N-oleyl-amide trihydrochloride, -arginyl-2,3-diaminopropionic acid-N-lauryl-N-myristyl-amide trihydrochloride or arginyl-lysine-N-lauryl-N-myristyl-amide trihydrochloride in combination with DPhyPE, wherein the content of DPhyPE is about 80 mol %, 65 mol %, 50 mol % and 35 mol % of the overall lipid content of the LNP. In some embodiments, the LNP includes -arginyl-2,3-diamino propionic acid-N-pahnityl-N-oleyl-amide trihydrochloride (lipid) and 1,2-diphytanoyl-sn-glycero-3-phosphoethanolamine (helper lipid). In some embodiments, the LNP includes -arginyl-2,3-diamino propionic acid-N-palmityl-N-oleyl-amide trihydrochloride (lipid), 1,2-diphytanoyl-sn-glycero-3-phosphoethanolamine (first helper lipid), and 1,2-disteroyl-sn-glycero-3-phosphoethanolamine-PEG2000 (second helper lipid).

In some embodiments, the second helper lipid is between about 0.05 mol % to 4.9 mol % or between about 1 mol % to 3 mol % of the total lipid content. In some embodiments, the LNP includes lipids at between about 45 mol % to 50 mol % of the total lipid content, a first helper lipid between about 45 mol % to 50 mol % of the total lipid content, under the proviso that there is a PEGylated second helper lipid between about 0.1 mol % to 5 mol %, between about 1 mol % to 4 mol %, or at about 2 mol % of the total lipid content, wherein the sum of the content of the lipids, the first helper lipid, and of the second helper lipid is 100 mol % of the total lipid content and wherein the sum of the first helper lipid and the second helper lipid is 50 mol % of the total lipid content. In some embodiments, the LNP comprises: (a) 50 mol % of -arginyl-2,3-diamino propionic acid-N-palmityl-N-oleyl-amide trihydrochloride, 48 mol % of 1,2-diphytanoyl-sn-glycero-3-phosphoethanolamine; and 2 mol % 1,2-distearoyl-sn-glycero-3-phosphoethanolamine-PEG2000; or (b) 50 mol % of -arginyl-2,3-diamino propionic acid-N-palmityl-N-oleyl-amide trihydrocloride, 49 mol % 1,2-diphytanoyl-sn-glycero-3-phosphoethanolamine; and 1 mol % N(Carbonyl-methoxypolyethylenglycol-2000)-1,2-distearoyl-sn-glycero3-phosphoethanolamine, or a sodium salt thereof.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP includes at least one lipid, a first helper lipid, and a shielding compound that is removable from the lipid composition under in vivo conditions. In some embodiments, the LNP also includes a second helper lipid. In some embodiments, the first helper lipid is ceramide. In some embodiments, the second helper lipid is ceramide. In some embodiments, the ceramide comprises at least one short carbon chain substituent of from 6 to 10 carbon atoms. In some embodiments, the ceramide comprises 8 carbon atoms. In some embodiments, the shielding compound is attached to a ceramide. In some embodiments, the shielding compound is attached to a ceramide. In some embodiments, the shielding compound is covalently attached to the ceramide. In some embodiments, the shielding compound is attached to a nucleic acid in the LNP. In some embodiments, the shielding compound is covalently attached to the nucleic acid. In some embodiments, the shielding compound is attached to the nucleic acid by a linker. In some embodiments, the linker is cleaved under physiological conditions. In some embodiments, the linker is selected from ssRNA, ssDNA, dsRNA, dsDNA, peptide, S-S-linkers and pH sensitive linkers. In some embodiments, the linker moiety is attached to the 3′ end of the sense strand of the nucleic acid. In some embodiments, the shielding compound comprises a pH-sensitive linker or a pH-sensitive moiety. In some embodiments, the pH-sensitive linker or pH-sensitive moiety is an anionic linker or an anionic moiety. In some embodiments, the anionic linker or anionic moiety is less anionic or neutral in an acidic environment. In some embodiments, the pH-sensitive linker or the pH-sensitive moiety is selected from the oligo (glutamic acid), oligophenolate(s) and diethylene triamine penta acetic acid.

In any of the LNP embodiments in the previous paragraph, the LNP can have an osmolality between about 50 to 600 mosmole/kg, between about 250 to 350 mosmole/kg, or between about 280 to 320 mosmole/kg, and/or wherein the LNP formed by the lipid and/or one or two helper lipids and the shielding compound have a particle size between about 20 to 200 nm, between about 30 to 100 nm, or between about 40 to 80 nm.

In some embodiments, the shielding compound provides for a longer circulation time in vivo and allows for a better biodistribution of the nucleic acid containing LNP. In some embodiments, the shielding compound prevents immediate interaction of the LNP with serum compounds or compounds of other bodily fluids or cytoplasma membranes, e.g., cytoplasma membranes of the endothelial lining of the vasculature, into which the LNP is administered. Additionally or alternatively, in some embodiments, the shielding compounds also prevent elements of the immune system from immediately interacting with the LNP. Additionally or alternatively, in some embodiments, the shielding compound acts as an anti-opsonizing compound. Without wishing to be bound by any mechanism or theory, in some embodiments, the shielding compound forms a cover or coat that reduces the surface area of the LNP available for interaction with its environment. Additionally or alternatively, in some embodiments, the shielding compound shields the overall charge of the LNP.

In another embodiment, the LNP includes at least one cationic lipid having Formula VI:

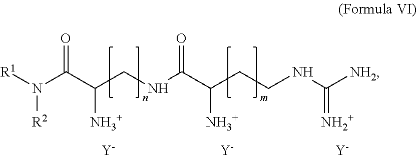

wherein n is 1, 2, 3, or 4, wherein m is 1, 2, or 3, wherein Y- is anion, wherein each of R1 and R2 is individually and independently selected from the group consisting of linear C12-C18 alkyl and linear C12-C18 alkenyl, a sterol compound, wherein the sterol compound is selected from the group consisting of cholesterol and stigmasterol, and a PEGylated lipid, wherein the PEGylated lipid comprises a PEG moiety, wherein the PEGylated lipid is selected from the group consisting of:
a PEGylated phosphoethanolamine of Formula VII:

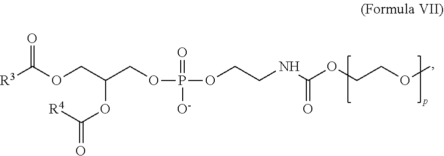

wherein R3 and R4 are individually and independently linear C13-C17 alkyl, and p is any integer between 15 to 130;
a PEGylated ceramide of Formula VIII:

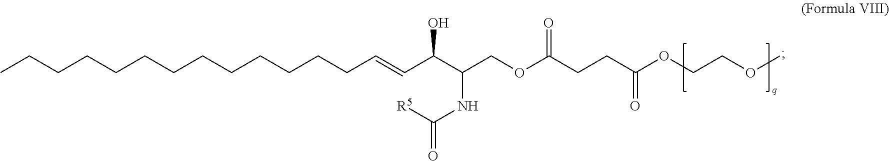

wherein R5 is linear C7-C15 alkyl, and q is any number between 15 to 130; and a PEGylated diacylglycerol of Formula IX:

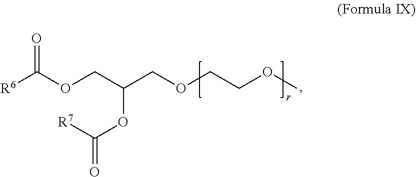

wherein each of R6 and R7 is individually and independently linear C11-C17 alkyl, and r is any integer from 15 to 130.

In some embodiments, R1 and R2 are different from each other. In some embodiments, R1 is palmityl and R2 is oleyl. In some embodiments, R1 is lauryl and R2 is myristyl. In some embodiments, R1 and R2 are the same. In some embodiments, each of R1 and R2 is individually and independently selected from the group consisting of C12 alkyl, C14 alkyl, C16 alkyl, C18 alkyl, C12 alkenyl, C14 alkenyl, C16 alkenyl and C18 alkenyl. In some embodiments, each of C12 alkenyl, C14 alkenyl, C16 alkenyl and C1 8 alkenyl comprises one or two double bonds. In some embodiments, C18 alkenyl is C18 alkenyl with one double bond between C9 and C10. In some embodiments, C18 alkenyl is cis-9-octadecyl.

In some embodiments, the cationic lipid is a compound of Formula X:

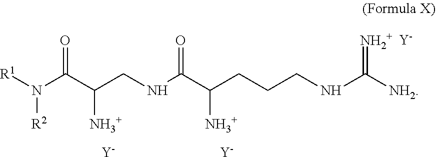

In some embodiments, Y- is selected from halogenids, acetate and trifluoroacetate. In some embodiments, the cationic lipid is -arginyl-2,3-diamino propionic acid-N-palmityl-N-oleyl-amide trihydrochloride of Formula III:

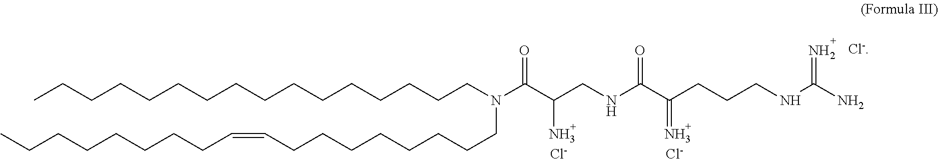

In some embodiments, the cationic lipid is -arginyl-2,3-diamino propionic acid-N-lauryl-N-myristyl-amide trihydrochloride of Formula IV:

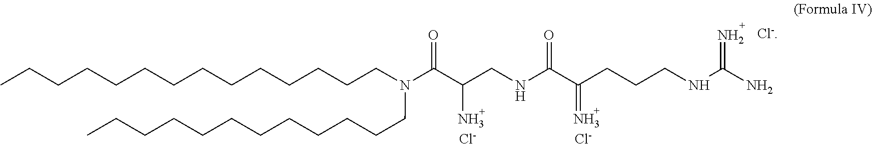

In some embodiments, the cationic lipid is -arginyl-lysine-N-lauryl-N-myristyl-amide trihydrochloride of Formula V:

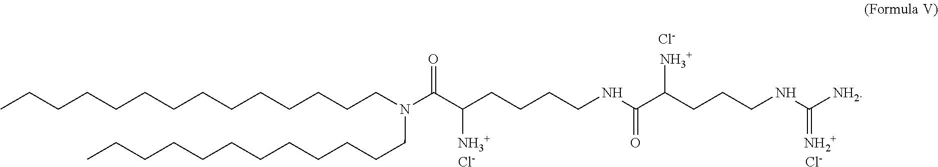 [SMILES: [Cl-] ; CCCCCCCCCCCCCCN(CCCCCCCCCCCC)C(=O)C([NH3+])CCCCNC(=O)C([NH3+])CCCNC(N)=[NH2+] ; [Cl-] ; [Cl-]]

In some embodiments, the sterol compound is cholesterol. In some embodiments, the sterol compound is stigmasterin.

In some embodiments, the PEG moiety of the PEGylated lipid has a molecular weight from about 800 to 5,000 Da. In some embodiments, the molecular weight of the PEG moiety of the PEGylated lipid is about 800 Da. In some embodiments, the molecular weight of the PEG moiety of the PEGylated lipid is about 2,000 Da. In some embodiments, the molecular weight of the PEG moiety of the PEGylated lipid is about 5,000 Da. In some embodiments, the PEGylated lipid is a PEGylated phosphoethanolamine of Formula VII, wherein each of R3 and R4 is individually and independently linear C13-C17 alkyl, and p is any integer from 18, 19 or 20, or from 44, 45 or 46 or from 113, 114 or 115. In some embodiments, R3 and R4 are the same. In some embodiments, R3 and R4 are different. In some embodiments, each of R3 and R4 is individually and independently selected from the group consisting of C13 alkyl, C15 alkyl and C17 alkyl. In some embodiments, the PEGylated phosphoethanolamine of Formula VII is 1,2-distearoyl-sn-glycero-3-phosphoethanolamine-N-[methoxy(polyethylene glycol)-2000](ammonium salt):

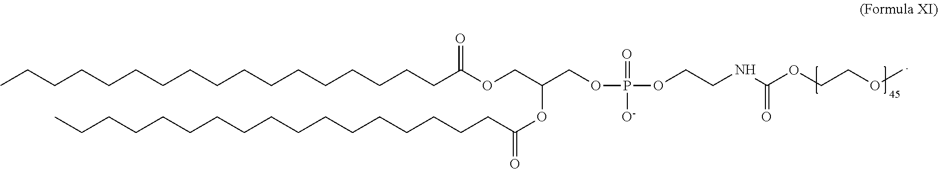

In some embodiments, the PEGylated phosphoethanolamine of Formula VII is 1,2-distearoyl-sn-glycero-3-phosphoethanolamine-N-[methoxy(polyethylene glycol)-5000](ammonium salt):

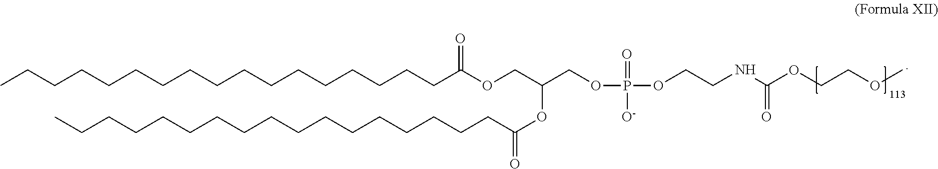

In some embodiments, the PEGylated lipid is a PEGylated ceramide of Formula VIII, wherein R5 is linear C7-C15 alkyl, and q is any integer from 18, 19 or 20, or from 44, 45 or 46 or from 113, 114 or 115. In some embodiments, R5 is linear C7 alkyl. In some embodiments, R5 is linear C15 alkyl. In some embodiments, the PEGylated ceramide of Formula VIII is N-octanoyl-sphingosine-1-{succinyl[methoxy(polyethylene glycol)2000]}:

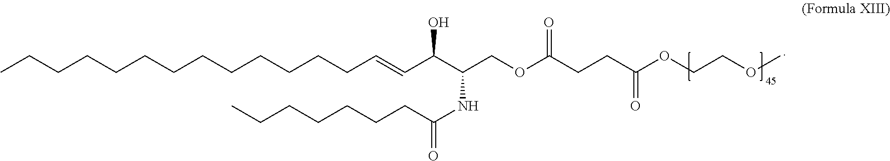

In some embodiments, the PEGylated ceramide of Formula VIII is N-palmitoyl-sphingosine-1-{succinyl[methoxy(polyethylene glycol)2000]}

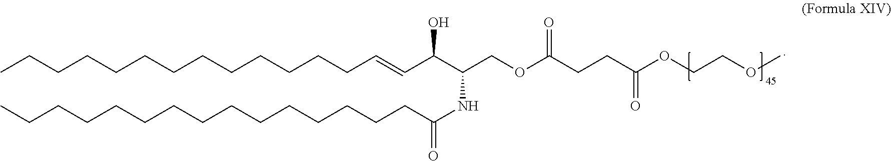

In some embodiments, the PEGylated lipid is a PEGylated diacylglycerol of Formula IX, wherein each of R6 and R7 is individually and independently linear C11-C17 alkyl, and r is any integer from 18, 19 or 20, or from 44, 45 or 46 or from 113, 114 or 115. In some embodiments, R6 and R7 are the same. In some embodiments, R6 and R7 are different. In some embodiments, each of R6 and R7 is individually and independently selected from the group consisting of linear C17 alkyl, linear C15 alkyl and linear C13 alkyl. In some embodiments, the PEGylated diacylglycerol of Formula IX 1,2-Distearoyl-sn-glycerol[methoxy(polyethylene glycol)2000]:

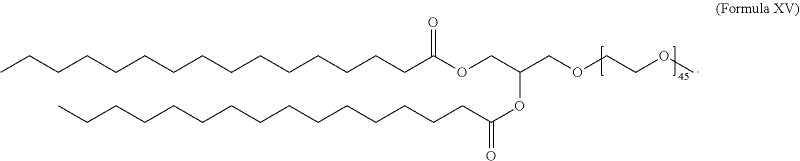

In some embodiments, the PEGylated diacylglycerol of Formula IX is 1,2-Dipalmitoyl-sn-glycerol[methoxy(polyethylene glycol)2000]:

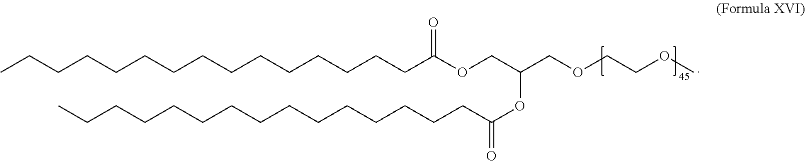

In some embodiments, the PEGylated diacylglycerol of Formula IX is:

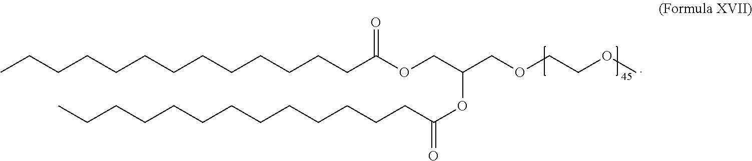

In some embodiments, the LNP includes at least one cationic lipid selected from of Formulas III, IV, and V, at least one sterol compound selected from a cholesterol and stigmasterin, and wherein the PEGylated lipid is at least one selected from Formulas XI and XII. In some embodiments, the LNP includes at least one cationic lipid selected from Formulas III, IV, and V, at least one sterol compound selected from a cholesterol and stigmasterin, and wherein the PEGylated lipid is at least one selected from Formulas XIII and XIV. In some embodiments, the LNP includes at least one cationic lipid selected from Formulas III, IV, and V, at least one sterol compound selected from a cholesterol and stigmasterin, and wherein the PEGylated lipid is at least one selected from Formulas XV and XVI. In some embodiments, the LNP includes a cationic lipid of Formula III, a cholesterol as the sterol compound, and wherein the PEGylated lipid is Formula XI.

In any of the LNP embodiments in the previous paragraph, wherein the content of the cationic lipid composition is between about 65 mole % to 75 mole %, the content of the sterol compound is between about 24 mole % to 34 mole % and the content of the PEGylated lipid is between about 0.5 mole % to 1.5 mole %, wherein the sum of the content of the cationic lipid, of the sterol compound and of the PEGylated lipid for the lipid composition is 100 mole %. In some embodiments, the cationic lipid is about 70 mole %, the content of the sterol compound is about 29 mole % and the content of the PEGylated lipid is about 1 mole %. In some embodiments, the LNP is 70 mole % of Formula III, 29 mole % of cholesterol, and 1 mole % of Formula XI.

Exosomes

Exosomes are endogenous nano-vesicles that transport RNAs and proteins, and which can deliver RNA to the brain and other target organs. To reduce immunogenicity, Alvarez-Erviti et al. (2011, Nat Biotechnol 29: 341) used self-derived dendritic cells for exosome production. Targeting to the brain was achieved by engineering the dendritic cells to express Lamp2b, an exosomal membrane protein, fused to the neuron-specific RVG peptide. Purified exosomes were loaded with exogenous RNA by electroporation. Intravenously injected RVG-targeted exosomes delivered GAPDH siRNA specifically to neurons, microglia, oligodendrocytes in the brain, resulting in a specific gene knockdown. Pre-exposure to RVG exosomes did not attenuate knockdown, and non-specific uptake in other tissues was not observed. The therapeutic potential of exosome-mediated siRNA delivery was demonstrated by the strong mRNA (60%) and protein (62%) knockdown of BACE1, a therapeutic target in Alzheimer's disease.

To obtain a pool of immunologically inert exosomes, Alvarez-Erviti et al. harvested bone marrow from inbred C57BL/6 mice with a homogenous major histocompatibility complex (MHC) haplotype. As immature dendritic cells produce large quantities of exosomes devoid of T-cell activators such as MHC-II and CD86, Alvarez-Erviti et al. selected for dendritic cells with granulocyte/macrophage-colony stimulating factor (GM-CSF) for 7 d. Exosomes were purified from the culture supernatant the following day using well-established ultracentrifugation protocols. The exosomes produced were physically homogenous, with a size distribution peaking at 80 nm in diameter as determined by nanoparticle tracking analysis (NTA) and electron microscopy. Alvarez-Erviti et al. obtained 6-12 μg of exosomes (measured based on protein concentration) per 106 cells.

Next, Alvarez-Erviti et al. investigated the possibility of loading modified exosomes with exogenous cargoes using electroporation protocols adapted for nanoscale applications. As electroporation for membrane particles at the nanometer scale is not well-characterized, nonspecific Cy5-labeled RNA was used for the empirical optimization of the electroporation protocol. The amount of encapsulated RNA was assayed after ultracentrifugation and lysis of exosomes. Electroporation at 400 V and 125 μF resulted in the greatest retention of RNA and was used for all subsequent experiments.

Alvarez-Erviti et al. administered 150 μg of each BACE1 siRNA encapsulated in 150 g of RVG exosomes to normal C57BL/6 mice and compared the knockdown efficiency to four controls: untreated mice, mice injected with RVG exosomes only, mice injected with BACE1 siRNA complexed to an in vivo cationic liposome reagent and mice injected with BACE1 siRNA complexed to RVG-9R, the RVG peptide conjugated to 9 D-arginines that electrostatically binds to the siRNA. Cortical tissue samples were analyzed 3 d after administration and a significant protein knockdown (45%, P<0.05, versus 62%, P<0.01) in both siRNA-RVG-9R-treated and siRNARVG exosome-treated mice was observed, resulting from a significant decrease in BACE1 mRNA levels (66% [+ or −] 15%, P<0.001 and 61% [+ or −] 13% respectively, P<0.01). Moreover, Applicants demonstrated a significant decrease (55%, P<0.05) in the total[beta]-amyloid 1-42 levels, a main component of the amyloid plaques in Alzheimer's pathology, in the RVG-exosome-treated animals. The decrease observed was greater than the 0-amyloid 1-40 decrease demonstrated in normal mice after intraventricular injection of BACE1 inhibitors. Alvarez-Erviti et al. carried out 5′-rapid amplification of cDNA ends (RACE) on BACE1 cleavage product, which provided evidence of RNAi-mediated knockdown by the siRNA.

Finally, Alvarez-Erviti et al. investigated whether RNA-RVG exosomes induced immune responses in vivo by assessing IL-6, IP-10, TNFα and IFN-α serum concentrations. Following exosome treatment, nonsignificant changes in all cytokines were registered similar to siRNA-transfection reagent treatment in contrast to siRNA-RVG-9R, which potently stimulated IL-6 secretion, confirming the immunologically inert profile of the exosome treatment. Given that exosomes encapsulate only 20% of siRNA, delivery with RVG-exosome appears to be more efficient than RVG-9R delivery as comparable mRNA knockdown and greater protein knockdown was achieved with fivefold less siRNA without the corresponding level of immune stimulation. This experiment demonstrated the therapeutic potential of RVG-exosome technology, which is potentially suited for long-term silencing of genes related to neurodegenerative diseases. The exosome delivery system of Alvarez-Erviti et al. may be applied to deliver the CRISPR-Cas system of the present invention to therapeutic targets, especially neurodegenerative diseases. A dosage of about 100 to 1000 mg of CRISPR Cas encapsulated in about 100 to 1000 mg of RVG exosomes may be contemplated for the present invention.

El-Andaloussi et al. (Nature Protocols 7, 2112-2126(2012)) discloses how exosomes derived from cultured cells can be harnessed for delivery of RNA in vitro and in vivo. This protocol first describes the generation of targeted exosomes through transfection of an expression vector, comprising an exosomal protein fused with a peptide ligand. Next, El-Andaloussi et al. explain how to purify and characterize exosomes from transfected cell supernatant. Next, El-Andaloussi et al. detail crucial steps for loading RNA into exosomes. Finally, El-Andaloussi et al. outline how to use exosomes to efficiently deliver RNA in vitro and in vivo in mouse brain. Examples of anticipated results in which exosome-mediated RNA delivery is evaluated by functional assays and imaging are also provided. The entire protocol takes ˜3 weeks. Delivery or administration according to the invention may be performed using exosomes produced from self-derived dendritic cells. From the herein teachings, this can be employed in the practice of the invention.

In another embodiment, the plasma exosomes of Wahlgren et al. (Nucleic Acids Research, 2012, Vol. 40, No. 17 e130) are contemplated. Exosomes are nano-sized vesicles (30-90 nm in size) produced by many cell types, including dendritic cells (DC), B cells, T cells, mast cells, epithelial cells and tumor cells. These vesicles are formed by inward budding of late endosomes and are then released to the extracellular environment upon fusion with the plasma membrane. Because exosomes naturally carry RNA between cells, this property may be useful in gene therapy, and from this disclosure can be employed in the practice of the instant invention.

Exosomes from plasma can be prepared by centrifugation of buffy coat at 900 g for 20 min to isolate the plasma followed by harvesting cell supernatants, centrifuging at 300 g for 10 min to eliminate cells and at 16 500 g for 30 min followed by filtration through a 0.22 mm filter. Exosomes are pelleted by ultracentrifugation at 120 000 g for 70 min. Chemical transfection of siRNA into exosomes is carried out according to the manufacturer's instructions in RNAi Human/Mouse Starter Kit (Quiagen, Hilden, Germany). siRNA is added to 100 ml PBS at a final concentration of 2 mmol/ml. After adding HiPerFect transfection reagent, the mixture is incubated for 10 min at RT. In order to remove the excess of micelles, the exosomes are re-isolated using aldehyde/sulfate latex beads. The chemical transfection of CRISPR Cas into exosomes may be conducted similarly to siRNA. The exosomes may be co-cultured with monocytes and lymphocytes isolated from the peripheral blood of healthy donors. Therefore, it may be contemplated that exosomes containing CRISPR Cas may be introduced to monocytes and lymphocytes of and autologously reintroduced into a human. Accordingly, delivery or administration according to the invention may be performed using plasma exosomes.

Liposomes

The lipid, lipid particle, or lipid bylayer or lipid entity of the invention can be prepared by methods well known in the art. See Wang et al., ACS Synthetic Biology, 1, 403-07 (2012); Wang et al., PNAS, 113(11) 2868-2873 (2016); Manoharan, et al., WO 2008/042973; Zugates et al., U.S. Pat. No. 8,071,082; Xu et al., WO 2014/186366 A1 (US20160082126). Xu et provides a way to make a nanocomplex for the delivery of saporin wherein the nanocomplex comprising saporin and a lipid-like compound, and wherein the nanocomplex has a particle size of 50 nm to 1000 nm; the saporin binds to the lipid-like compound via non-covalent interaction or covalent bonding; and the lipid-like compound has a hydrophilic moiety, a hydrophobic moiety, and a linker joining the hydrophilic moiety and the hydrophobic moiety, the hydrophilic moiety being optionally charged and the hydrophobic moiety having 8 to 24 carbon atoms. Xu et al., WO 2014/186348 (US20160129120) provides examples of nanocomplexes of modified peptides or proteins comprising a cationic delivery agent and an anionic pharmaceutical agent, wherein the nanocomplex has a particle size of 50 to 1000 nm, the cationic delivery agent binds to the anionic pharmaceutical agent, and the anionic pharmaceutical agent is a modified peptide or protein formed of a peptide and a protein and an added chemical moiety that contains an anionic group. The added chemical moiety is linked to the peptide or protein via an amide group, an ester group, an ether group, a thioether group, a disulfide group, a hydrazone group, a sulfenate ester group, an amidine group, a urea group, a carbamate group, an imidoester group, or a carbonate group. More particularly these documents provide examples of lipid or lipid-like compounds that can be used to make the particle delivery system of the present invention, including compounds of the formula B1-K1-A-K2-B2, in which A, the hydrophilic moiety, is

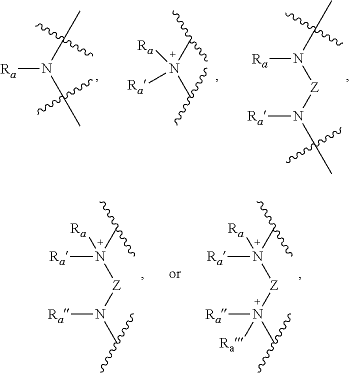

each of Ra, Ra′, Ra″, and Ra′″, independently, being a C1-C20 monovalent aliphatic radical, a C1-C20 0 monovalent heteroaliphatic radical, a monovalent aryl radical, or a monovalent heteroaryl radical; and Z being a C1-C20 bivalent aliphatic radical, a C1-C20 bivalent heteroaliphatic radical, a bivalent aryl radical, or a bivalent heteroaryl radical; each of BI, the hydrophobic moiety, and B2, also the hydrophobic moiety, independently, is a C12-20 aliphatic radical or a C12-20 heteroaliphatic radical; and each of K1, the linker, and K2, also the linker, independently, is O, S, Si, C1-C6 alkylene

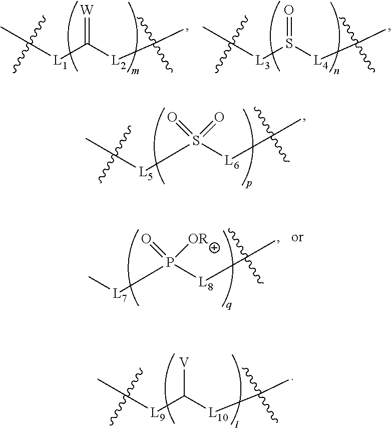

in which each of m, n, p, q, and t, independently, is 1-6; W is O, S, or NRC; each of L1, L3, L5, L7, and L9, independently, is a bond, O, S, or NRd; each of L2, L4, L6, L8, and L10, independently, is a bond, O, S, or NRe; and V is ORf, SRg, or NRhRi, each of Rb, Rc, Rd, Re, Rf, Rg, Rh, and Ri, independently, being H, OH, a C1-C10 oxyaliphatic radical, a C1-C10 monovalent aliphatic radical, a C1-C10 monovalent heteroaliphatic radical, a monovalent aryl radical, or a monovalent heteroaryl radical and specific compounds:

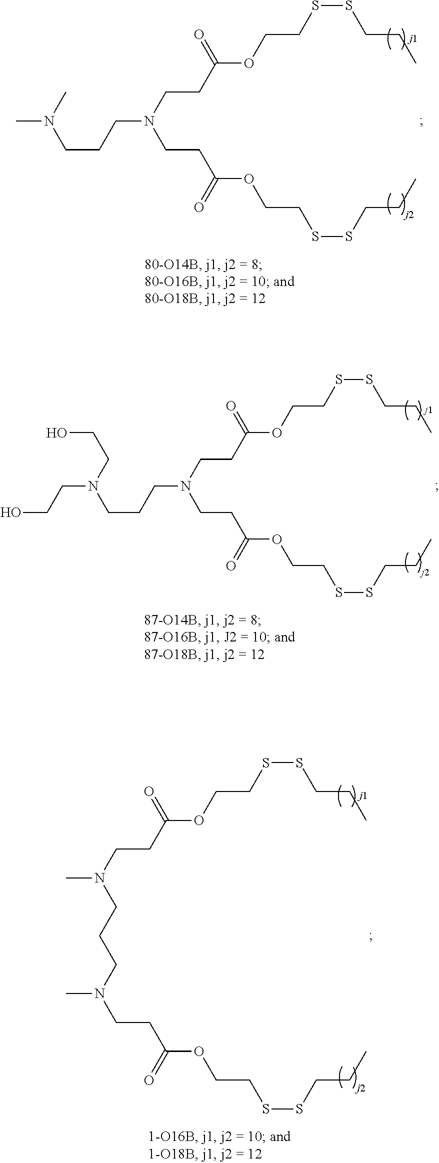

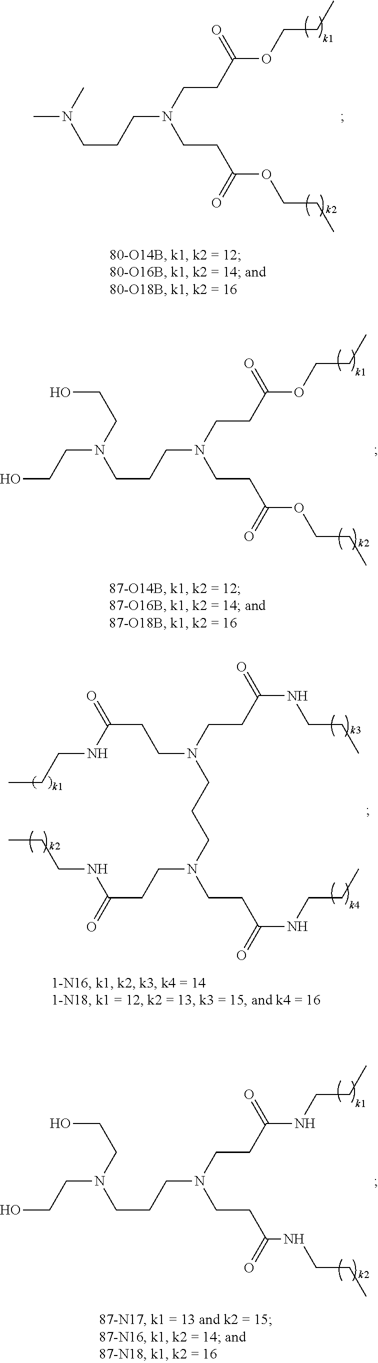

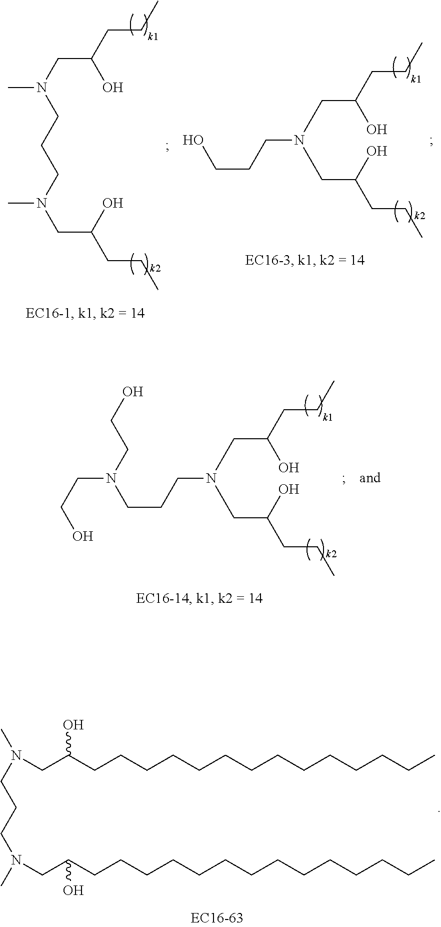

Additional examples of cationic lipid that can be used to make the particle delivery system of the invention can be found in US20150140070, wherein the cationic lipid has the formula

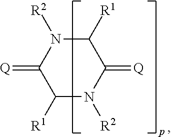

wherein p is an integer between 1 and 9, inclusive; each instance of Q is independently O, S, or NRQ; RQ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii) or (iii); each instance of R1 is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —ORA1, —N(RA1)2, —SRA1, or a group of formula:

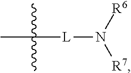

L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof, and each of R6 and R7 is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of formula (i), (ii) or (iii); each occurrence of RA1 is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two RA1 groups, together with the nitrogen atom to which they are attached, are joined to form an optionally substituted heterocyclic or optionally substituted heteroaryl ring; each instance of R2 is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); Formulae (i), (ii), and (iii) are:

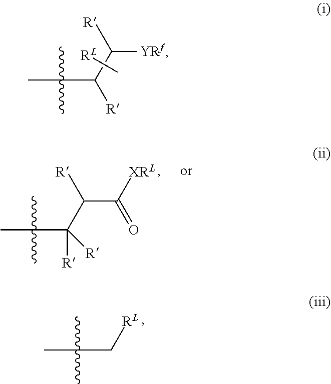

each instance of R′ is independently hydrogen or optionally substituted alkyl; X is O, S, or NRX; RX is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; Y is O, S, or NRY; RY is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; RP is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; RL is optionally substituted C1-50 alkyl, optionally substituted C2-50 alkenyl, optionally substituted C2-50 alkynyl, optionally substituted heteroC1-50 alkyl, optionally substituted heteroC2-50 alkenyl, optionally substituted heteroC2-50 alkynyl, or a polymer; provided that at least one instance of RQ, R2, R6, or R7 is a group of the formula (i), (ii), or (iii); in Liu et al., (US 20160200779, US 20150118216, US 20150071903, and US 20150071903), which provide examples of cationic lipids to include polyethylenimine, polyamidoamine (PAMAM) starburst dendrimers, Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3.beta.-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanamin-ium trifluoroacetate (DOSPA), 1,2-dimyristyloxypropyl-3-dimethyl-hydroxyethyl ammonium bromide; and dimethyldioctadecylammonium bromide (DDAB); in WO2013/093648 which provides cationic lipids of formula

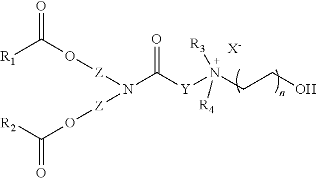

in which Z=an alkyl linker, C2-C4 alkyl, Y=an alkyl linker, C1-C6 alkyl, R1 and R2 are each independently C10-C30alkyl, C10-C30alkenyl, or C10-C30alkynyl, C10-C30alkyl, C10-C20alkyl, C12-C18alkyl, C13-C17alkyl, C13alkyl, C10-C30alkenyl, C10-C20oalkenyl. C12-C18alkenyl, C13-C17alkenyl, C17alkenyl; R3 and R4 are each independently hydrogen, C1-C6 alkyl, or -CH2CH2OH, C1-C6 alkyl, C1-C3alkyl; n is 1-6; and X is a counterion, including any nitrogen counterion, as that term is readily understood in the art, and specific cationic lipids including

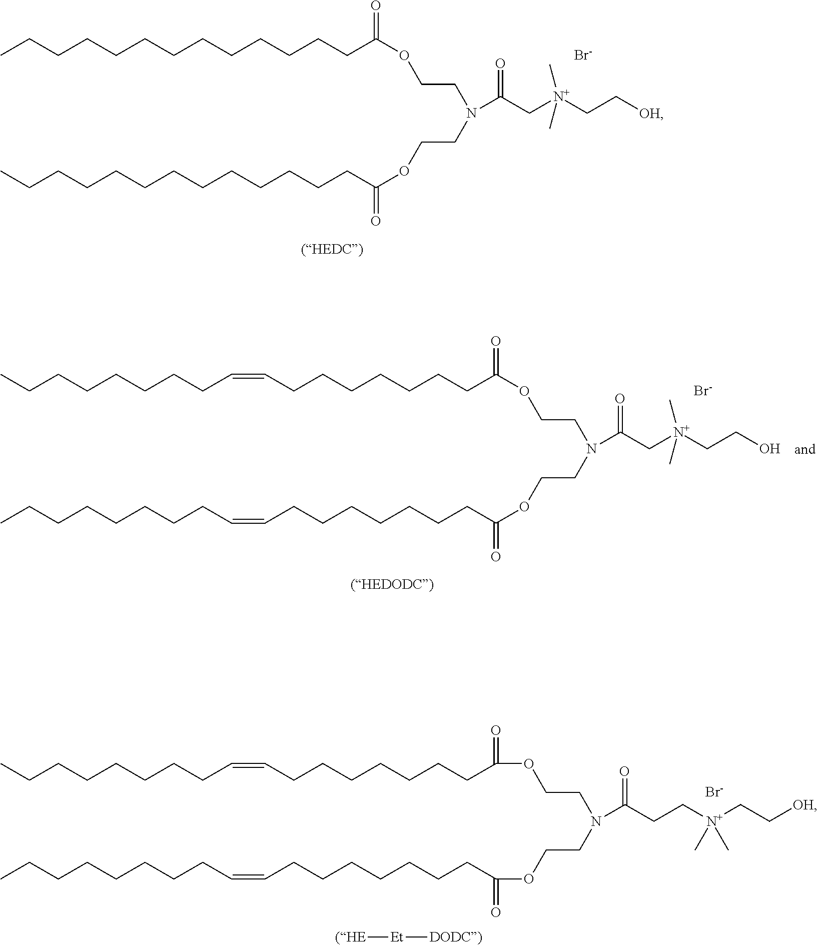

WO2013/093648 also provides examples of other cationic charged lipids at physiological pH including N,N-dioleyl-N,N-dimethylammonium chloride (DODAC), N,N-distearyl-N,N-dimethylammonium bromide (DDAB); N-(1,2-dimyristyloxyprop-3-yl)-N,N-dimethyl-N-hydroxyethylammonium bromide (DMRIE) and dioctadecylamidoglycyl carboxyspermidine (DOGS); in US 20160257951, which provides cationic lipids with a general formula

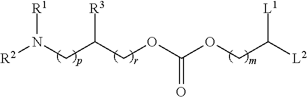

or a pharmacologically acceptable salt thereof, wherein R1 and R2 are each independently a hydrogen atom, a C1-C6 alkyl group optionally substituted with one or more substituents selected from substituent group a, a C2-C6 alkenyl group optionally substituted with one or more substituents selected from substituent group a, a C2-C6 alkynyl group optionally substituted with one or more substituents selected from substituent group a, or a C3-C7 cycloalkyl group optionally substituted with one or more substituents selected from substituent group a, or R1 and R2 form a 3- to 10-membered heterocyclic ring together with the nitrogen atom bonded thereto, wherein the heterocyclic ring is optionally substituted with one or more substituents selected from substituent group a and optionally contains one or more atoms selected from a nitrogen atom, an oxygen atom, and a sulfur atom, in addition to the nitrogen atom bonded to R1 and R2, as atoms constituting the heterocyclic ring; R8 is a hydrogen atom or a C1-C6 alkyl group optionally substituted with one or more substituents selected from substituent group a; or R1 and R8 together are the group -(CH2)q-; substituent group a consists of a halogen atom, an oxo group, a hydroxy group, a sulfanyl group, an amino group, a cyano group, a C1-C6 alkyl group, a C1-C6 halogenated alkyl group, a C1-C6 alkoxy group, a C1-C6 alkylsulfanyl group, a C1-C6 alkylamino group, and a C1-C7 alkanoyl group; L1 is a C10-C24 alkyl group optionally substituted with one or more substituents selected from substituent group 31, a C10-C24 alkenyl group optionally substituted with one or more substituents selected from substituent group 31, a C3-C24 alkynyl group optionally substituted with one or more substituents selected from substituent group 31, or a (C1-C10 alkyl)-(Q)k-(C1-C10 alkyl) group optionally substituted with one or more substituents selected from substituent group 31; L2 is, independently of L1, a C10-C24 alkyl group optionally substituted with one or more substituents selected from substituent group 31, a C10-C24 alkenyl group optionally substituted with one or more substituents selected from substituent group 31, a C3-C24 alkynyl group optionally substituted with one or more substituents selected from substituent group 31, a (C1-C10 alkyl)-(Q)k-(C1-C10 alkyl) group optionally substituted with having one or more substituents selected from substituent group 31, a (C10-C24 alkoxy)methyl group optionally substituted with one or more substituents selected from substituent group 31, a (C10-C24 alkenyl)oxymethyl group optionally substituted with one or more substituents selected from substituent group 31, a (C3-C24 alkynyl)oxymethyl group optionally substituted with one or more substituents selected from substituent group P31, or a (C1-C10 alkyl)-(Q)k-(C1-C10 alkoxy)methyl group optionally substituted with one or more substituents selected from substituent group 31; substituent group P31 consists of a halogen atom, an oxo group, a cyano group, a C1-C6 alkyl group, a C1-C6 halogenated alkyl group, a C1-C6 alkoxy group, a C1-C6 alkylsulfanyl group, a C1-C7 alkanoyl group, a C1-C7 alkanoyloxy group, a C3-C7 alkoxyalkoxy group, a (C1-C6 alkoxy)carbonyl group, a (C1-C6 alkoxy)carboxyl group, a (C1-C6 alkoxy)carbamoyl group, and a (C1-C6 alkylamino)carboxyl group; Q is a group of formula:

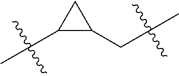 [SMILES: *CC1CC1*]

when L1 and L2 are each substituted with one or more substituents selected from substituent group 31 and substituent group 131 is a C1-C6 alkyl group, a C1-C6 alkoxy group, a C1-C6 alkylsulfanyl group, a C1-C7 alkanoyl group, or a C1-C7 alkanoyloxy group, the substituent or substituents selected from substituent group p31 in L1 and the substituent or substituents selected from substituent group 31 in L2 optionally bind to each other to form a cyclic structure; k is 1, 2, 3, 4, 5, 6, or 7; m is 0 or 1; p is O, 1, or 2; q is 1, 2, 3, or 4; and r is O, 1, 2, or 3, provided that p+r is 2 or larger, or q+r is 2 or larger, and specific cationic lipids including

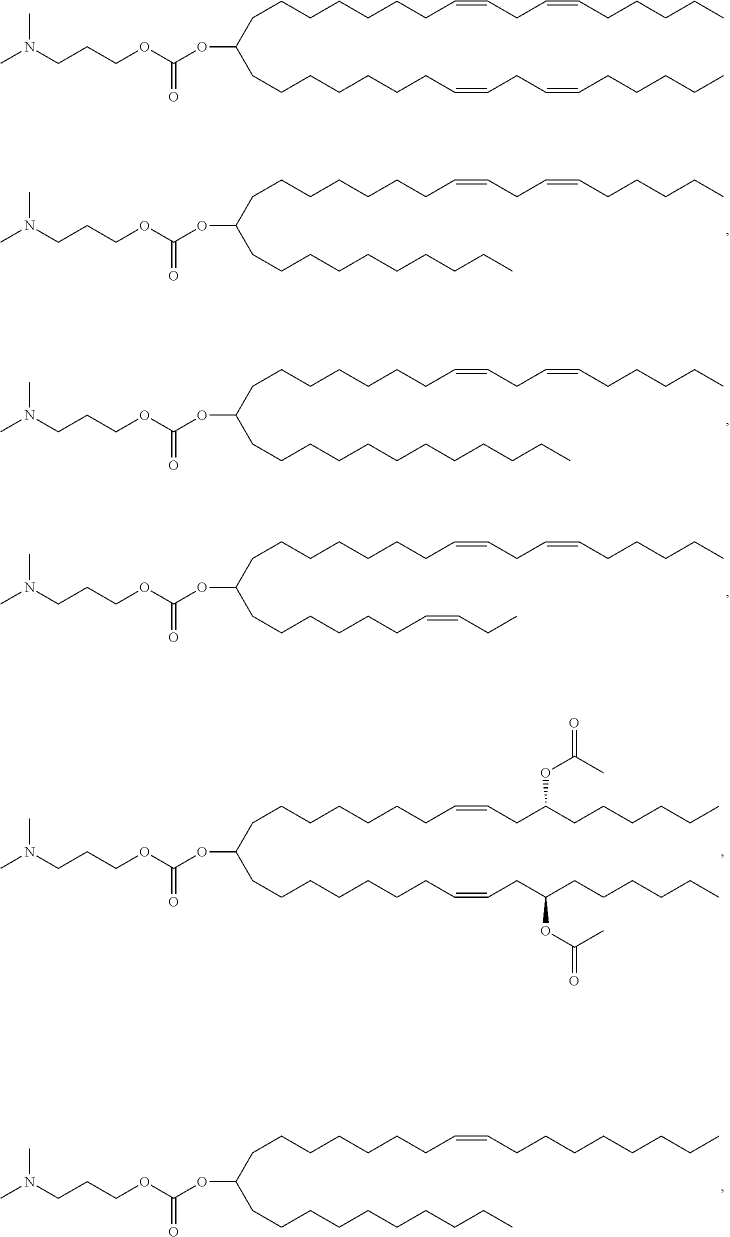

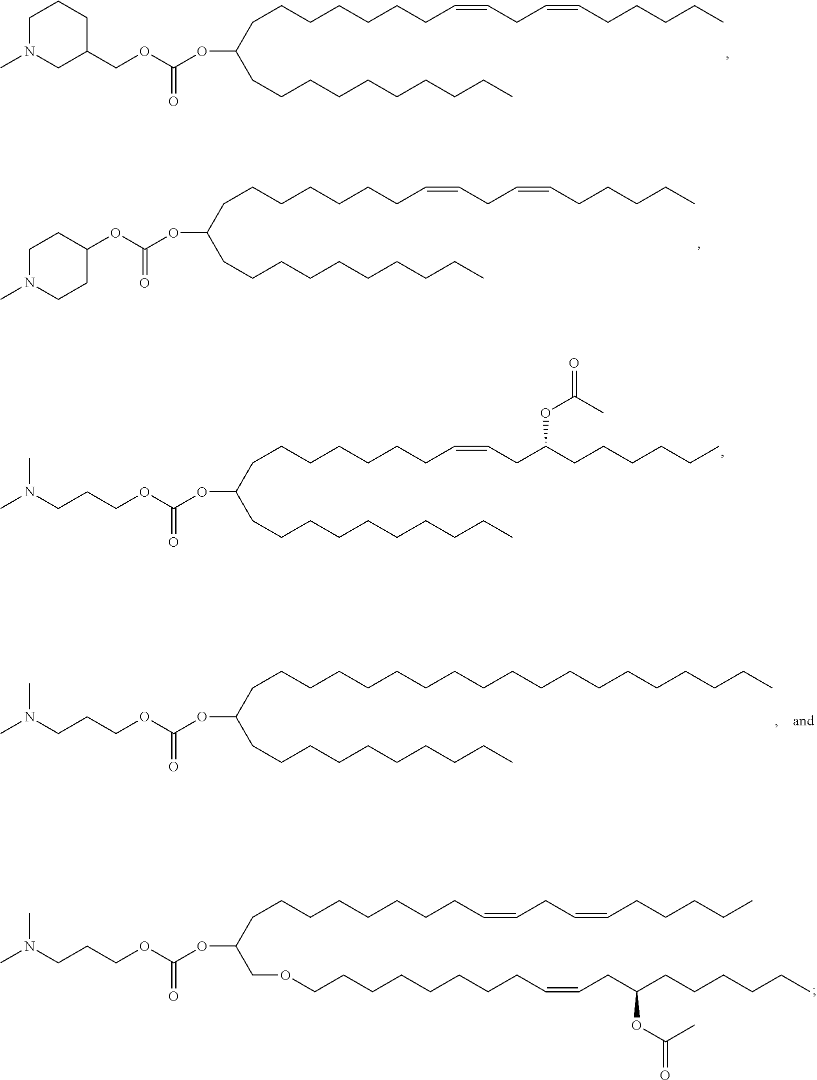

and in US 20160244761, which provides cationic lipids that include 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 1,2-di-. gamma.-linolenyloxy-N,N-dimethylaminopropane (.gamma.-DLenDMA), 1,2-dilinoleyloxy-keto-N,N-dimethyl-3-aminopropane (DLin-K-DMA), 1,2-dilinoleyl-4-(2-dimethylaminoethyl)-[1,3]-dioxolane (DLin-K—C2-DMA) (also known as DLin-C2K-DMA, XTC2, and C2K), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K—C4-DMA), 1,2-dilinolenyloxy-4-(2-dimethylaminoethyl)-[1,3]-dioxolane (DLen-C2K-DMA), 1,2-di-.gamma.-linolenyloxy-4-(2-dimethylaminoethyl)-[1,3]-dioxolane (.gamma.-DLen-C2K-DMA), dilinoleylmethyl-3-dimethylaminopropionate (DLin-M-C2-DMA) (also known as MC2), (6Z,9Z,28Z,31Z)-heptatriaconta-6,9,28,31-tetraen-19-yl 4-(dimethylamino) butanoate (DLin-M-C3-DMA) (also known as MC3) and 3-(dilinoleylmethoxy)-N,N-dimethylpropan-1-amine (DLin-MP-DMA) (also known as 1-Bl 1).

In one embodiment, the lipid compound is preferably a bio-reducible material, e.g., a bio-reducible polymer and a bio-reducible lipid-like compound.

In embodiment, the lipid compound comprises a hydrophilic head, and a hydrophobic tail, and optionally a linker.

In one embodiment, the hydrophilic head contains one or more hydrophilic functional groups, e.g., hydroxyl, carboxyl, amino, sulfhydryl, phosphate, amide, ester, ether, carbamate, carbonate, carbamide and phosphodiester. These groups can form hydrogen bonds and are optionally positively or negatively charged, in particular at physiological conditions such as physiological pH.

In one embodiment, the hydrophobic tail is a saturated or unsaturated, linear or branched, acyclic or cyclic, aromatic or nonaromatic hydrocarbon moiety, wherein the saturated or unsaturated, linear or branched, acyclic or cyclic, aromatic or nonaromatic hydrocarbon moiety optionally contains a disulfide bond and/or 8-24 carbon atoms. One or more of the carbon atoms can be replaced with a heteroatom, such as N, O, P, B, S, Si, Sb, A1, Sn, As, Se, and Ge. The lipid or lipid-like compounds containing disulfide bond can be bioreducible.

In one embodiment, the linker of the lipid or lipid-like compound links the hydrophilic head and the hydrophobic tail. The linker can be any chemical group that is hydrophilic or hydrophobic, polar or non-polar, e.g., O, S, Si, amino, alkylene, ester, amide, carbamate, carbamide, carbonate phosphate, phosphite, sulfate, sulfite, and thiosulfate.

The lipid or lipid-like compounds described above include the compounds themselves, as well as their salts and solvates, if applicable. A salt, for example, can be formed between an anion and a positively charged group (e.g., amino) on a lipid-like compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a lipid-like compound. Suitable cations include sodium ion, potassium ion, magnesium ion, calcium ion, and an ammonium cation such as tetramethylammonium ion. The lipid-like compounds also include those salts containing quaternary nitrogen atoms. A solvate refers to a complex formed between a lipid-like compound and a pharmaceutically acceptable solvent. Examples of pharmaceutically acceptable solvents include water, ethanol, isopropanol, ethyl acetate, acetic acid, and ethanolamine.

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-dioleoyl-sn-glycero-3-phosphoethanolamine (DOPE) increases the stability (see, e.g., Spuch and Navarro, Journal of Drug Delivery, vol. 2011, Article ID 469679, 12 pages, 2011. doi:10.1155/2011/469679 for review).

In a particularly advantageous embodiment, Trojan Horse liposomes (also known as Molecular Trojan Horses) are desirable and protocols may be found at cshprotocols.cshlp.org/content/2010/4/pdb.prot5407.long. These particles allow delivery of a transgene to the entire brain after an intravascular injection. Without being bound by limitation, it is believed that neutral lipid particles with specific antibodies conjugated to surface allow crossing of the blood brain barrier via endocytosis. Applicant postulates utilizing Trojan Horse Liposomes to deliver the CRISPR family of nucleases to the brain via an intravascular injection, which would allow whole brain transgenic animals without the need for embryonic manipulation. About 1-5 g of DNA or RNA may be contemplated for in vivo administration in liposomes.

In another embodiment, the CRISPR Cas system or components thereof may be administered in liposomes, such as a stable nucleic-acid-lipid particle (SNALP) (see, e.g., Morrissey et al., Nature Biotechnology, Vol. 23, No. 8, August 2005). Daily intravenous injections of about 1, 3 or 5 mg/kg/day of a specific CRISPR Cas targeted in a SNALP are contemplated. The daily treatment may be over about three days and then weekly for about five weeks. In another embodiment, a specific CRISPR Cas encapsulated SNALP) administered by intravenous injection to at doses of about 1 or 2.5 mg/kg are also contemplated (see, e.g., Zimmerman et al., Nature Letters, Vol. 441, 4 May 2006). The SNALP formulation may contain the lipids 3-N-[(wmethoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio (see, e.g., Zimmerman et al., Nature Letters, Vol. 441, 4 May 2006).

In another embodiment, stable nucleic-acid-lipid particles (SNALPs) have proven to be effective delivery molecules to highly vascularized HepG2-derived liver tumors but not in poorly vascularized HCT-116 derived liver tumors (see, e.g., Li, Gene Therapy (2012) 19, 775-780). The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulted SNALP liposomes are about 80-100 nm in size.

In yet another embodiment, a SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane (see, e.g., Geisbert et al., Lancet 2010; 375: 1896-905). A dosage of about 2 mg/kg total CRISPR Cas per dose administered as, for example, a bolus intravenous infusion may be contemplated.

In yet another embodiment, a SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA) (see, e.g., Judge, J. Clin. Invest. 119:661-673 (2009)). Formulations used for in vivo studies may comprise a final lipid/RNA mass ratio of about 9:1.

The safety profile of RNAi nanomedicines has been reviewed by Barros and Gollob of Alnylam Pharmaceuticals (see, e.g., Advanced Drug Delivery Reviews 64 (2012) 1730-1737). The stable nucleic acid lipid particle (SNALP) is comprised of four different lipids—an ionizable lipid (DLinDMA) that is cationic at low pH, a neutral helper lipid, cholesterol, and a diffusible polyethylene glycol (PEG)-lipid. The particle is approximately 80 nm in diameter and is charge-neutral at physiologic pH. During formulation, the ionizable lipid serves to condense lipid with the anionic RNA during particle formation. When positively charged under increasingly acidic endosomal conditions, the ionizable lipid also mediates the fusion of SNALP with the endosomal membrane enabling release of RNA into the cytoplasm. The PEG-lipid stabilizes the particle and reduces aggregation during formulation, and subsequently provides a neutral hydrophilic exterior that improves pharmacokinetic properties.

To date, two clinical programs have been initiated using SNALP formulations with RNA. Tekmira Pharmaceuticals recently completed a phase I single-dose study of SNALP-ApoB in adult volunteers with elevated LDL cholesterol. ApoB is predominantly expressed in the liver and jejunum and is essential for the assembly and secretion of VLDL and LDL. Seventeen subjects received a single dose of SNALP-ApoB (dose escalation across 7 dose levels). There was no evidence of liver toxicity (anticipated as the potential dose-limiting toxicity based on preclinical studies). One (of two) subjects at the highest dose experienced flu-like symptoms consistent with immune system stimulation, and the decision was made to conclude the trial.

Alnylam Pharmaceuticals has similarly advanced ALN-TTR01, which employs the SNALP technology described above and targets hepatocyte production of both mutant and wild-type TTR to treat TTR amyloidosis (ATTR). Three ATTR syndromes have been described: familial amyloidotic polyneuropathy (FAP) and familial amyloidotic cardiomyopathy (FAC) —both caused by autosomal dominant mutations in TTR; and senile systemic amyloidosis (SSA) cause by wildtype TTR. A placebo-controlled, single dose-escalation phase I trial of ALN-TTR01 was recently completed in patients with ATTR. ALN-TTR01 was administered as a 15-minute IV infusion to 31 patients (23 with study drug and 8 with placebo) within a dose range of 0.01 to 1.0 mg/kg (based on siRNA). Treatment was well tolerated with no significant increases in liver function tests. Infusion-related reactions were noted in 3 of 23 patients at ≥0.4 mg/kg; all responded to slowing of the infusion rate and all continued on study. Minimal and transient elevations of serum cytokines IL-6, IP-10 and IL-ira were noted in two patients at the highest dose of 1 mg/kg (as anticipated from preclinical and NHP studies). Lowering of serum TTR, the expected pharmacodynamics effect of ALN-TTR01, was observed at 1 mg/kg.

In yet another embodiment, a SNALP may be made by solubilizing a cationic lipid, DSPC, cholesterol and PEG-lipid e.g., in ethanol, e.g., at a molar ratio of 40:10:40:10, respectively (see, Semple et al., Nature Niotechnology, Volume 28 Number 2 Feb. 2010, pp. 172-177). The lipid mixture was added to an aqueous buffer (50 mM citrate, pH 4) with mixing to a final ethanol and lipid concentration of 30% (vol/vol) and 6.1 mg/ml, respectively, and allowed to equilibrate at 22° C. for 2 min before extrusion. The hydrated lipids were extruded through two stacked 80 nm pore-sized filters (Nuclepore) at 22° C. using a Lipex Extruder (Northern Lipids) until a vesicle diameter of 70-90 nm, as determined by dynamic light scattering analysis, was obtained. This generally required 1-3 passes. The siRNA (solubilized in a 50 mM citrate, pH 4 aqueous solution containing 30% ethanol) was added to the pre-equilibrated (35° C.) vesicles at a rate of −5 ml/min with mixing. After a final target siRNA/lipid ratio of 0.06 (wt/wt) was reached, the mixture was incubated for a further 30 min at 35° C. to allow vesicle reorganization and encapsulation of the siRNA. The ethanol was then removed and the external buffer replaced with PBS (155 mM NaCl, 3 mM Na2HPO4, 1 mM KH2PO4, pH 7.5) by either dialysis or tangential flow diafiltration. siRNA were encapsulated in SNALP using a controlled step-wise dilution method process. The lipid constituents of KC2-SNALP were DLin-KC2-DMA (cationic lipid), dipalmitoylphosphatidylcholine (DPPC; Avanti Polar Lipids), synthetic cholesterol (Sigma) and PEG-C-DMA used at a molar ratio of 57.1:7.1:34.3:1.4. Upon formation of the loaded particles, SNALP were dialyzed against PBS and filter sterilized through a 0.2 m filter before use. Mean particle sizes were 75-85 nm and 90-95% of the siRNA was encapsulated within the lipid particles. The final siRNA/lipid ratio in formulations used for in vivo testing was ˜0.15 (wt/wt). LNP-siRNA systems containing Factor VII siRNA were diluted to the appropriate concentrations in sterile PBS immediately before use and the formulations were administered intravenously through the lateral tail vein in a total volume of 10 ml/kg. This method and these delivery systems may be extrapolated to the CRISPR Cas system of the present invention.

Other Lipids

Other cationic lipids, such as amino lipid 2,2-dilinoleyl-4-dimethylaminoethyl-[1,3]-dioxolane (DLin-KC2-DMA) may be utilized to encapsulate CRISPR Cas or components thereof or nucleic acid molecule(s) coding therefor e.g., similar to SiRNA (see, e.g., Jayaraman, Angew. Chem. Int. Ed. 2012, 51, 8529-8533), and hence may be employed in the practice of the invention. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11+0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Michael S D Kormann et al. (“Expression of therapeutic proteins after delivery of chemically modified mRNA in mice: Nature Biotechnology, Volume:29, Pages: 154-157 (2011)) describes the use of lipid envelopes to deliver RNA. Use of lipid envelopes is also preferred in the present invention.

In another embodiment, lipids may be formulated with the CRISPR Cas system of the present invention or component(s) thereof or nucleic acid molecule(s) coding therefor to form lipid nanoparticles (LNPs). Lipids include, but are not limited to, DLin-KC2-DMA4, C12-200 and colipids disteroylphosphatidyl choline, cholesterol, and PEG-DMG may be formulated with CRISPR Cas instead of siRNA (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/di steroylphosphatidyl choline/cholesterol/PEG-DMG). The final lipid:siRNA weight ratio may be ˜12:1 and 9:1 in the case of DLin-KC2-DMA and C12-200 lipid nanoparticles (LNPs), respectively. The formulations may have mean particle diameters of −80 nm with >90% entrapment efficiency. A 3 mg/kg dose may be contemplated.

Tekmira has a portfolio of approximately 95 patent families, in the U.S. and abroad, that are directed to various aspects of LNPs and LNP formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316), all of which may be used and/or adapted to the present invention.

The CRISPR Cas system or components thereof or nucleic acid molecule(s) coding therefor may be delivered encapsulated in PLGA Microspheres such as that further described in US published applications 20130252281 and 20130245107 and 20130244279 (assigned to Moderna Therapeutics) which relate to aspects of formulation of compositions comprising modified nucleic acid molecules which may encode a protein, a protein precursor, or a partially or fully processed form of the protein or a protein precursor. The formulation may have a molar ratio 50:10:38.5:1.5-3.0 (cationic lipid:fusogenic lipid:cholesterol:PEG lipid). The PEG lipid may be selected from, but is not limited to PEG-c-DOMG, PEG-DMG. The fusogenic lipid may be DSPC. See also, Schrum et al., Delivery and Formulation of Engineered Nucleic Acids, US published application 20120251618.

Nanomerics' technology addresses bioavailability challenges for a broad range of therapeutics, including low molecular weight hydrophobic drugs, peptides, and nucleic acid based therapeutics (plasmid, siRNA, miRNA). Specific administration routes for which the technology has demonstrated clear advantages include the oral route, transport across the blood-brain-barrier, delivery to solid tumours, as well as to the eye. See, e.g., Mazza et al., 2013, ACS Nano. 2013 Feb. 26; 7(2):1016-26; Uchegbu and Siew, 2013, J Pharm Sci. 102(2):305-10 and Lalatsa et al., 2012, J Control Release. 2012 Jul. 20; 161(2):523-36.

US Patent Publication No. 20050019923 describes cationic dendrimers for delivering bioactive molecules, such as polynucleotide molecules, peptides and polypeptides and/or pharmaceutical agents, to a mammalian body. The dendrimers are suitable for targeting the delivery of the bioactive molecules to, for example, the liver, spleen, lung, kidney or heart (or even the brain). Dendrimers are synthetic 3-dimensional macromolecules that are prepared in a step-wise fashion from simple branched monomer units, the nature and functionality of which can be easily controlled and varied. Dendrimers are synthesised from the repeated addition of building blocks to a multifunctional core (divergent approach to synthesis), or towards a multifunctional core (convergent approach to synthesis) and each addition of a 3-dimensional shell of building blocks leads to the formation of a higher generation of the dendrimers. Polypropylenimine dendrimers start from a diaminobutane core to which is added twice the number of amino groups by a double Michael addition of acrylonitrile to the primary amines followed by the hydrogenation of the nitriles. This results in a doubling of the amino groups. Polypropylenimine dendrimers contain 100% protonable nitrogens and up to 64 terminal amino groups (generation 5, DAB 64). Protonable groups are usually amine groups which are able to accept protons at neutral pH. The use of dendrimers as gene delivery agents has largely focused on the use of the polyamidoamine. and phosphorous containing compounds with a mixture of amine/amide or N—P(O2)S as the conjugating units respectively with no work being reported on the use of the lower generation polypropylenimine dendrimers for gene delivery. Polypropylenimine dendrimers have also been studied as pH sensitive controlled release systems for drug delivery and for their encapsulation of guest molecules when chemically modified by peripheral amino acid groups. The cytotoxicity and interaction of polypropylenimine dendrimers with DNA as well as the transfection efficacy of DAB 64 has also been studied.

US Patent Publication No. 20050019923 is based upon the observation that, contrary to earlier reports, cationic dendrimers, such as polypropylenimine dendrimers, display suitable properties, such as specific targeting and low toxicity, for use in the targeted delivery of bioactive molecules, such as genetic material. In addition, derivatives of the cationic dendrimer also display suitable properties for the targeted delivery of bioactive molecules. See also, Bioactive Polymers, US published application 20080267903, which discloses “Various polymers, including cationic polyamine polymers and dendrimeric polymers, are shown to possess anti-proliferative activity, and may therefore be useful for treatment of disorders characterised by undesirable cellular proliferation such as neoplasms and tumours, inflammatory disorders (including autoimmune disorders), psoriasis and atherosclerosis. The polymers may be used alone as active agents, or as delivery vehicles for other therapeutic agents, such as drug molecules or nucleic acids for gene therapy. In such cases, the polymers' own intrinsic anti-tumour activity may complement the activity of the agent to be delivered.” The disclosures of these patent publications may be employed in conjunction with herein teachings for delivery of CRISPR Cas system(s) or component(s) thereof or nucleic acid molecule(s) coding therefor.

Supercharged Proteins

Supercharged proteins are a class of engineered or naturally occurring proteins with unusually high positive or negative net theoretical charge and may be employed in delivery of CRISPR Cas system(s) or component(s) thereof or nucleic acid molecule(s) coding therefor. Both supernegatively and superpositively charged proteins exhibit a remarkable ability to withstand thermally or chemically induced aggregation. Superpositively charged proteins are also able to penetrate mammalian cells. Associating cargo with these proteins, such as plasmid DNA, RNA, or other proteins, can enable the functional delivery of these macromolecules into mammalian cells both in vitro and in vivo. David Liu's lab reported the creation and characterization of supercharged proteins in 2007 (Lawrence et al., 2007, Journal of the American Chemical Society 129, 10110-10112).

The nonviral delivery of RNA and plasmid DNA into mammalian cells are valuable both for research and therapeutic applications (Akinc et al., 2010, Nat. Biotech. 26, 561-569). Purified+36 GFP protein (or other superpositively charged protein) is mixed with RNAs in the appropriate serum-free media and allowed to complex prior addition to cells. Inclusion of serum at this stage inhibits formation of the supercharged protein-RNA complexes and reduces the effectiveness of the treatment. The following protocol has been found to be effective for a variety of cell lines (McNaughton et al., 2009, Proc. Natl. Acad. Sci. USA 106, 6111-6116) (However, pilot experiments varying the dose of protein and RNA should be performed to optimize the procedure for specific cell lines):
(1) One day before treatment, plate 1×105 cells per well in a 48-well plate.
(2) On the day of treatment, dilute purified+36 GFP protein in serumfree media to a final concentration 200 nM. Add RNA to a final concentration of 50 nM. Vortex to mix and incubate at room temperature for 10 min.
(3) During incubation, aspirate media from cells and wash once with PBS.
(4) Following incubation of +36 GFP and RNA, add the protein-RNA complexes to cells.
(5) Incubate cells with complexes at 37° C. for 4h.
(6) Following incubation, aspirate the media and wash three times with 20 U/mL heparin PBS. Incubate cells with serum-containing media for a further 48h or longer depending upon the assay for activity.
(7) Analyze cells by immunoblot, qPCR, phenotypic assay, or other appropriate method.

David Liu's lab has further found +36 GFP to be an effective plasmid delivery reagent in a range of cells. As plasmid DNA is a larger cargo than siRNA, proportionately more +36 GFP protein is required to effectively complex plasmids. For effective plasmid delivery Applicants have developed a variant of +36 GFP bearing a C-terminal HA2 peptide tag, a known endosome-disrupting peptide derived from the influenza virus hemagglutinin protein. The following protocol has been effective in a variety of cells, but as above it is advised that plasmid DNA and supercharged protein doses be optimized for specific cell lines and delivery applications:

(1) One day before treatment, plate 1×105 per well in a 48-well plate. (2) On the day of treatment, dilute purified [symbol] 36 GFP protein in serumfree media to a final concentration 2 mM. Add 1 mg of plasmid DNA. Vortex to mix and incubate at room temperature for 10 min.

(3) During incubation, aspirate media from cells and wash once with PBS.

(4) Following incubation of [symbol] 36 GFP and plasmid DNA, gently add the protein-DNA complexes to cells.

(5) Incubate cells with complexes at 37 C for 4h.

(6) Following incubation, aspirate the media and wash with PBS. Incubate cells in serum-containing media and incubate for a further 24-48h.

(7) Analyze plasmid delivery (e.g., by plasmid-driven gene expression) as appropriate.

See also, e.g., McNaughton et al., Proc. Natl. Acad. Sci. USA 106, 6111-6116 (2009); Cronican et al., ACS Chemical Biology 5, 747-752 (2010); Cronican et al., Chemistry & Biology 18, 833-838 (2011); Thompson et al., Methods in Enzymology 503, 293-319 (2012); Thompson, D. B., et al., Chemistry & Biology 19 (7), 831-843 (2012). The methods of the super charged proteins may be used and/or adapted for delivery of the CRISPR Cas system of the present invention. These systems of Dr. Lui and documents herein in conjunction with herein teaching can be employed in the delivery of CRISPR Cas system(s) or component(s) thereof or nucleic acid molecule(s) coding therefor.

Cell Penetrating Peptides (CPPs)

In yet another embodiment, cell penetrating peptides (CPPs) are contemplated for the delivery of the CRISPR Cas system. CPPs are short peptides that facilitate cellular uptake of various molecular cargo (from nanosize particles to small chemical molecules and large fragments of DNA). The term “cargo” as used herein includes but is not limited to the group consisting of therapeutic agents, diagnostic probes, peptides, nucleic acids, antisense oligonucleotides, plasmids, proteins, particles, including nanoparticles, liposomes, chromophores, small molecules and radioactive materials. In aspects of the invention, the cargo may also comprise any component of the CRISPR Cas system or the entire functional CRISPR Cas system. Aspects of the present invention further provide methods for delivering a desired cargo into a subject comprising: (a) preparing a complex comprising the cell penetrating peptide of the present invention and a desired cargo, and (b) orally, intraarticularly, intraperitoneally, intrathecally, intrarterially, intranasally, intraparenchymally, subcutaneously, intramuscularly, intravenously, dermally, intrarectally, or topically administering the complex to a subject. The cargo is associated with the peptides either through chemical linkage via covalent bonds or through non-covalent interactions.

The function of the CPPs are to deliver the cargo into cells, a process that commonly occurs through endocytosis with the cargo delivered to the endosomes of living mammalian cells. Cell-penetrating peptides are of different sizes, amino acid sequences, and charges but all CPPs have one distinct characteristic, which is the ability to translocate the plasma membrane and facilitate the delivery of various molecular cargoes to the cytoplasm or an organelle. CPP translocation may be classified into three main entry mechanisms: direct penetration in the membrane, endocytosis-mediated entry, and translocation through the formation of a transitory structure. CPPs have found numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots. CPPs hold great potential as in vitro and in vivo delivery vectors for use in research and medicine. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. One of the initial CPPs discovered was the trans-activating transcriptional activator (Tat) from Human Immunodeficiency Virus 1 (HIV-1) which was found to be efficiently taken up from the surrounding media by numerous cell types in culture. Since then, the number of known CPPs has expanded considerably and small molecule synthetic analogues with more effective protein transduction properties have been generated. CPPs include but are not limited to Penetratin, Tat (48-60), Transportan, and (R-AhX-R4) (Ahx=aminohexanoyl).

U.S. Pat. No. 8,372,951, provides a CPP derived from eosinophil cationic protein (ECP) which exhibits highly cell-penetrating efficiency and low toxicity. Aspects of delivering the CPP with its cargo into a vertebrate subject are also provided. Further aspects of CPPs and their delivery are described in U.S. Pat. Nos. 8,575,305; 8,614,194 and 8,044,019. CPPs can be used to deliver the CRISPR-Cas system or components thereof. That CPPs can be employed to deliver the CRISPR-Cas system or components thereof is also provided in the manuscript “Gene disruption by cell-penetrating peptide-mediated delivery of Cas9 protein and guide RNA”, by Suresh Ramakrishna, Abu-Bonsrah Kwaku Dad, Jagadish Beloor, et al. Genome Res. 2014 Apr. 2. [Epub ahead of print], incorporated by reference in its entirety, wherein it is demonstrated that treatment with CPP-conjugated recombinant Cas9 protein and CPP-complexed guide RNAs lead to endogenous gene disruptions in human cell lines. In the paper the Cas9 protein was conjugated to CPP via a thioether bond, whereas the guide RNA was complexed with CPP, forming condensed, positively charged particles. It was shown that simultaneous and sequential treatment of human cells, including embryonic stem cells, dermal fibroblasts, HEK293T cells, HeLa cells, and embryonic carcinoma cells, with the modified Cas9 and guide RNA led to efficient gene disruptions with reduced off-target mutations relative to plasmid transfections.

Implantable Devices

In another embodiment, implantable devices are also contemplated for delivery of the CRISPR Cas system or component(s) thereof or nucleic acid molecule(s) coding therefor. For example, US Patent Publication 20110195123 discloses an implantable medical device which elutes a drug locally and in prolonged period is provided, including several types of such a device, the treatment modes of implementation and methods of implantation. The device comprising of polymeric substrate, such as a matrix for example, that is used as the device body, and drugs, and in some cases additional scaffolding materials, such as metals or additional polymers, and materials to enhance visibility and imaging. An implantable delivery device can be advantageous in providing release locally and over a prolonged period, where drug is released directly to the extracellular matrix (ECM) of the diseased area such as tumor, inflammation, degeneration or for symptomatic objectives, or to injured smooth muscle cells, or for prevention. One kind of drug is RNA, as disclosed above, and this system may be used/and or adapted to the CRISPR Cas system of the present invention. The modes of implantation in some embodiments are existing implantation procedures that are developed and used today for other treatments, including brachytherapy and needle biopsy. In such cases the dimensions of the new implant described in this invention are similar to the original implant. Typically a few devices are implanted during the same treatment procedure.

US Patent Publication 20110195123, provides a drug delivery implantable or insertable system, including systems applicable to a cavity such as the abdominal cavity and/or any other type of administration in which the drug delivery system is not anchored or attached, comprising a biostable and/or degradable and/or bioabsorbable polymeric substrate, which may for example optionally be a matrix. It should be noted that the term “insertion” also includes implantation. The drug delivery system is preferably implemented as a “Loder” as described in US Patent Publication 20110195123.

The polymer or plurality of polymers are biocompatible, incorporating an agent and/or plurality of agents, enabling the release of agent at a controlled rate, wherein the total volume of the polymeric substrate, such as a matrix for example, in some embodiments is optionally and preferably no greater than a maximum volume that permits a therapeutic level of the agent to be reached. As a non-limiting example, such a volume is preferably within the range of 0.1 m3 to 1000 mm3, as required by the volume for the agent load. The Loder may optionally be larger, for example when incorporated with a device whose size is determined by functionality, for example and without limitation, a knee joint, an intra-uterine or cervical ring and the like.

The drug delivery system (for delivering the composition) is designed in some embodiments to preferably employ degradable polymers, wherein the main release mechanism is bulk erosion; or in some embodiments, non degradable, or slowly degraded polymers are used, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the surface is preferably maintained effectively constant during a significant period of the total drug releasing period, and therefore the diffusion rate is effectively constant (termed “zero mode” diffusion). By the term “constant” it is meant a diffusion rate that is preferably maintained above the lower threshold of therapeutic effectiveness, but which may still optionally feature an initial burst and/or may fluctuate, for example increasing and decreasing to a certain degree. The diffusion rate is preferably so maintained for a prolonged period, and it can be considered constant to a certain level to optimize the therapeutically effective period, for example the effective silencing period.

The drug delivery system optionally and preferably is designed to shield the nucleotide based therapeutic agent from degradation, whether chemical in nature or due to attack from enzymes and other factors in the body of the subject.

The drug delivery system of US Patent Publication 20110195123 is optionally associated with sensing and/or activation appliances that are operated at and/or after implantation of the device, by non and/or minimally invasive methods of activation and/or acceleration/deceleration, for example optionally including but not limited to thermal heating and cooling, laser beams, and ultrasonic, including focused ultrasound and/or RF (radiofrequency) methods or devices.

According to some embodiments of US Patent Publication 20110195123, the site for local delivery may optionally include target sites characterized by high abnormal proliferation of cells, and suppressed apoptosis, including tumors, active and or chronic inflammation and infection including autoimmune diseases states, degenerating tissue including muscle and nervous tissue, chronic pain, degenerative sites, and location of bone fractures and other wound locations for enhancement of regeneration of tissue, and injured cardiac, smooth and striated muscle.

The site for implantation of the composition, or target site, preferably features a radius, area and/or volume that is sufficiently small for targeted local delivery. For example, the target site optionally has a diameter in a range of from about 0.1 mm to about 5 cm.

The location of the target site is preferably selected for maximum therapeutic efficacy. For example, the composition of the drug delivery system (optionally with a device for implantation as described above) is optionally and preferably implanted within or in the proximity of a tumor environment, or the blood supply associated thereof.

For example the composition (optionally with the device) is optionally implanted within or in the proximity to pancreas, prostate, breast, liver, via the nipple, within the vascular system and so forth.

The target location is optionally selected from the group comprising, consisting essentially of, or consisting of (as non-limiting examples only, as optionally any site within the body may be suitable for implanting a Loder): 1. brain at degenerative sites like in Parkinson or Alzheimer disease at the basal ganglia, white and gray matter; 2. spine as in the case of amyotrophic lateral sclerosis (ALS); 3. uterine cervix to prevent HPV infection; 4. active and chronic inflammatory joints; 5. dermis as in the case of psoriasis; 6. sympathetic and sensoric nervous sites for analgesic effect; 7. Intra osseous implantation; 8. acute and chronic infection sites; 9. Intra vaginal; 10. Inner ear-auditory system, labyrinth of the inner ear, vestibular system; 11. Intra tracheal; 12. Intra-cardiac; coronary, epicardiac; 13. urinary bladder; 14. biliary system; 15. parenchymal tissue including and not limited to the kidney, liver, spleen; 16. lymph nodes; 17. salivary glands; 18. dental gums; 19. Intra-articular (into joints); 20. Intra-ocular; 21. Brain tissue; 22. Brain ventricles; 23. Cavities, including abdominal cavity (for example but without limitation, for ovary cancer); 24. Intra esophageal and 25. Intra rectal.

Optionally insertion of the system (for example a device containing the composition) is associated with injection of material to the ECM at the target site and the vicinity of that site to affect local pH and/or temperature and/or other biological factors affecting the diffusion of the drug and/or drug kinetics in the ECM, of the target site and the vicinity of such a site.

Optionally, according to some embodiments, the release of said agent could be associated with sensing and/or activation appliances that are operated prior and/or at and/or after insertion, by non and/or minimally invasive and/or else methods of activation and/or acceleration/deceleration, including laser beam, radiation, thermal heating and cooling, and ultrasonic, including focused ultrasound and/or RF (radiofrequency) methods or devices, and chemical activators.

According to other embodiments of US Patent Publication 20110195123, the drug preferably comprises a RNA, for example for localized cancer cases in breast, pancreas, brain, kidney, bladder, lung, and prostate as described below. Although exemplified with RNAi, many drugs are applicable to be encapsulated in Loder, and can be used in association with this invention, as long as such drugs can be encapsulated with the Loder substrate, such as a matrix for example, and this system may be used and/or adapted to deliver the CRISPR Cas system of the present invention.

As another example of a specific application, neuro and muscular degenerative diseases develop due to abnormal gene expression. Local delivery of RNAs may have therapeutic properties for interfering with such abnormal gene expression. Local delivery of anti apoptotic, anti inflammatory and anti degenerative drugs including small drugs and macromolecules may also optionally be therapeutic. In such cases the Loder is applied for prolonged release at constant rate and/or through a dedicated device that is implanted separately. All of this may be used and/or adapted to the CRISPR Cas system of the present invention.

As yet another example of a specific application, psychiatric and cognitive disorders are treated with gene modifiers. Gene knockdown is a treatment option. Loders locally delivering agents to central nervous system sites are therapeutic options for psychiatric and cognitive disorders including but not limited to psychosis, bi-polar diseases, neurotic disorders and behavioral maladies. The Loders could also deliver locally drugs including small drugs and macromolecules upon implantation at specific brain sites. All of this may be used and/or adapted to the CRISPR Cas system of the present invention.

As another example of a specific application, silencing of innate and/or adaptive immune mediators at local sites enables the prevention of organ transplant rejection. Local delivery of RNAs and immunomodulating reagents with the Loder implanted into the transplanted organ and/or the implanted site renders local immune suppression by repelling immune cells such as CD8 activated against the transplanted organ. All of this may be used/and or adapted to the CRISPR Cas system of the present invention.

As another example of a specific application, vascular growth factors including VEGFs and angiogenin and others are essential for neovascularization. Local delivery of the factors, peptides, peptidomimetics, or suppressing their repressors is an important therapeutic modality; silencing the repressors and local delivery of the factors, peptides, macromolecules and small drugs stimulating angiogenesis with the Loder is therapeutic for peripheral, systemic and cardiac vascular disease.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as ERCP, stereotactic methods into the brain tissue, Laparoscopy, including implantation with a laparoscope into joints, abdominal organs, the bladder wall and body cavities.

Implantable devices may also include cells, such as epidermal progenitor cells that have been edited or modified to express the CRISPR-Cas systems disclosed herein and embedded with an implantable device, such as a patch. See. Yue et al. “Engineered Epidermal Progenitor Cells Can Correct Diet-Induced Obesity and Diabetes” Cell Stem Cell (2017) 21(2):256-263.

Implantable device technology herein discussed can be employed with herein teachings and hence by this disclosure and the knowledge in the art, CRISPR-Cas system or components thereof or nucleic acid molecules thereof or encoding or providing components may be delivered via an implantable device.

Aerosol Delivery

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector.

Hybrid Viral Capsid Delivery Systems

In one aspect, the invention provides a particle delivery system comprising a hybrid virus capsid protein or hybrid viral outer protein, wherein the hybrid virus capsid or outer protein comprises a virus capsid or outer protein attached to at least a portion of a non-capsid protein or peptide. The genetic material of a virus is stored within a viral structure called the capsid. The capsid of certain viruses are enclosed in a membrane called the viral envelope. The viral envelope is made up of a lipid bilayer embedded with viral proteins including viral glycoproteins. As used herein, an “envelope protein” or “outer protein” means a protein exposed at the surface of a viral particle that is not a capsid protein. For example envelope or outer proteins typically comprise proteins embedded in the envelope of the virus. Non-limiting examples of outer or envelope proteins include, without limit, gp41 and gpl20 of HIV, hemagglutinin, neuraminidase and M2 proteins of influenza virus.

In one example embodiment of the delivery system, the non-capsid protein or peptide has a molecular weight of up to a megadalton, or has a molecular weight in the range of 110 to 160 kDa, 160 to 200 kDa, 200 to 250 kDa, 250 to 300 kDa, 300 to 400 kDa, or 400 to 500 kDa, the non-capsid protein or peptide comprises a CRISPR protein.

The present application provides a vector for delivering an effector protein and at least one CRISPR guide RNA to a cell comprising a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the virus is an adeno-associated virus (AAV) or an adenovirus. In another embodiment, the effector protein is a CRISPR anzyme. In a further embodiment, the CRISPR enzyme is SaCas9, Cpf1, Cas13b or C2c2.

In a related aspect, the invention provides a lentiviral vector for delivering an effector protein and at least one CRISPR guide RNA to a cell comprising a promoter operably linked to a polynucleotide sequence encoding Cpf1 and a second promoter operably linked to a polynucleotide sequence encoding at least one guide RNA, wherein the polynucleotide sequences are in reverse orientation.

In an embodiment of the delivery system, the virus is lentivirus or murine leukemia virus (MuMLV).

In an embodiment of the delivery system, the virus is an Adenoviridae or a Parvoviridae or a retrovirus or a Rhabdoviridae or an enveloped virus having a glycoprotein protein (G protein).

In an embodiment of the delivery system, the virus is VSV or rabies virus.

In an embodiment of the delivery system, the capsid or outer protein comprises a capsid protein having VP1, VP2 or VP3.

In an embodiment of the delivery system, the capsid protein is VP3, and the non-capsid protein is inserted into or attached to VP3 loop 3 or loop 6.

In an embodiment of the delivery system, the virus is delivered to the interior of a cell.

In an embodiment of the delivery system, the capsid or outer protein and the non-capsid protein can dissociate after delivery into a cell.

In an embodiment of the delivery system, the capsid or outer protein is attached to the protein by a linker.

In an embodiment of the delivery system, the linker comprises amino acids.

In an embodiment of the delivery system, the linker is a chemical linker.

In an embodiment of the delivery system, the linker is cleavable.

In an embodiment of the delivery system, the linker is biodegradable.

In an embodiment of the delivery system, the linker comprises (GGGGS)1-3 (SEQ ID Nos. 3 and 4), ENLYFQG (SEQ ID No. 5), or a disulfide.

In an embodiment, the delivery system comprises a protease or nucleic acid molecule(s) encoding a protease that is expressed, said protease being capable of cleaving the linker, whereby there can be cleavage of the linker. In an embodiment of the invention, a protease is delivered with a particle component of the system, for example packaged, mixed with, or enclosed by lipid and or capsid. Entry of the particle into a cell is thereby accompanied or followed by cleavage and dissociation of payload from particle. In certain embodients, an expressible nucleic acid encoding a protease is delivered, whereby at entry or following entry of the particle into a cell, there is protease expression, linker cleavage, and dissociation of payload from capsid. In certain embodiments, dissociation of payload occurs with viral replication. In certain embodiments, dissociation of payload occurs in the absence of productive virus replication.

In an embodiment of the delivery system, each terminus of a CRISPR protein is attached to the capsid or outer protein by a linker.

In an embodiment of the delivery system, the non-capsid protein is attached to the exterior portion of the capsid or outer protein.

In an embodiment of the delivery system, the non-capsid protein is attached to the interior portion of the capsid or outer protein.

In an embodiment of the delivery system, the capsid or outer protein and the non-capsid protein are a fusion protein.

In an embodiment of the delivery system, the non-capsid protein is encapsulated by the capsid or outer protein.

In an embodiment of the delivery system, the non-capsid protein is attached to a component of the capsid protein or a component of the outer protein prior to formation of the capsid or the outer protein.

In an embodiment of the delivery system, the protein is attached to the capsid or outer protein after formation of the capsid or outer protein.

In an embodiment, the delivery system comprises a targeting moiety, such as active targeting of a lipid entity of the invention, e.g., lipid particle or nanoparticle or liposome or lipid bylayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm. 13.118 (2013), and the documents it cites, all of which are incorporated herein by reference. Mention is also made of WO/2016/027264, and the documents it cites, all of which are incorporated herein by reference. And mention is made ofLorenzer et al, “Going beyond the liver: Progress and challenges of targeted delivery of siRNA therapeutics,” Journal of Controlled Release, 203: 1-15 (2015), , and the documents it cites, all of which are incorporated herein by reference.

An actively targeting lipid particle or nanoparticle or liposome or lipid bylayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bylayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bylayer). The microenvironment of a cell mass such as a tumor microenvironment can be targeted; for instance, it may be advantageous to target cell mass vasculature, such as the the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. ap3-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be been utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydro phobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe₃O₄ or y-Fe₂O₃, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a maj or structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the invention includes the delivery system comprising an actively targeting lipid particle or nanoparticle or liposome or lipid bilayer delivery system; or comprising a lipid particle or nanoparticle or liposome or lipid bylayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety. Likewise, the following table provides exemplary targeting moieties that can be used in the practice of the invention an as to each an aspect of the invention provides a delivery system that comprises such a targeting moiety.

| Targeting Moiety | Target Molecule | Target Cell or Tissue |
|---|---|---|
| folate | folate receptor | cancer cells |
| transferrin | transferrin | cancer cells |
| | receptor | |
| Antibody CC52 | rat CC531 | rat colon adenocarcinoma |
| | | CC531 |
| anti- HER2 antibody | HER2 | HER2 -overexpressing |
| | | tumors |
| anti-GD2 | GD2 | neuroblastoma, melanoma |
| anti-EGFR | EGFR | tumor cells overexpressing |
| | | EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell | leukemia, lymphoma |
| | marker) | |
| cell-penetrating peptide | | blood-brain barrier |
| cyclic arginine-glycine- | avβ3 | glioblastoma cells, |
| aspartic acid-tyrosine- | | human umbilical |
| cysteine peptide | | vein endothelial cells, |
| (c(RGDyC)-LP) | | tumor angiogenesis |
| ASSHN peptide | | endothelial progenitor |
| | | cells; anti-cancer |
| PR_♯b peptide | α5β1 integrin | cancer cells |
| AG86 peptide | α6β4 integrin | cancer cells |
| KCCYSL (P6.1 peptide) | HER-2 receptor | cancer cells |
| affinity peptide LN | Aminopeptidase | APN-positive tumor |
| (YEVGHRC) | N (APN/CD13) | |
| synthetic somatostatin | Somatostatin | breast cancer |
| analogue | receptor 2 | |
| | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the delivery system comprises a lipid entity having a targeting moiety.

In an embodiment of the delivery system, the protein comprises a CRISPR protein, or portion thereof.

In some embodiments a non-capsid protein or protein that is not a virus outer protein or a virus envelope (sometimes herein shorthanded as “non-capsid protein”), such as a CRISPR protein or portion thereof, can have one or more functional moiety(ies) thereon, such as a moiety for targeting or locating, such as an NLS or NES, or an activator or repressor.

In an embodiment of the delivery system, a protein or portion thereof can comprise a tag.

In an aspect, the invention provides a virus particle comprising a capsid or outer protein having one or more hybrid virus capsid or outer proteins comprising the virus capsid or outer protein attached to at least a portion of a non-capsid protein or a CRISPR protein.

In an aspect, the invention provides an in vitro method of delivery comprising contacting the delivery system with a cell, optionally a eukaryotic cell, whereby there is delivery into the cell of constituents of the delivery system.

In an aspect, the invention provides an in vitro, a research or study method of delivery comprising contacting the delivery system with a cell, optionally a eukaryotic cell, whereby there is delivery into the cell of constituents of the delivery system, obtaining data or results from the contacting, and transmitting the data or results.

In an aspect, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results.

In an aspect, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting.

In an embodiment, the cell product is non-human or animal.

In one aspect, the invention provides a particle delivery system comprising a composite virus particle, wherein the composite virus particle comprises a lipid, a virus capsid protein, and at least a portion of a non-capsid protein or peptide. The non-capsid peptide or protein can have a molecular weight of up to one megadalton.

In one embodiment, the particle delivery system comprises a virus particle adsorbed to a liposome or lipid particle or nanoparticle. In one embodiment, a virus is adsorbed to a liposome or lipid particle or nanoparticle either through electrostatic interactions, or is covalently linked through a linker. The lipid particle or nanoparticles (lmg/ml) dissolved in either sodium acetate buffer (pH 5.2) or pure H2O (pH 7) are positively charged. The isoelectropoint of most viruses is in the range of 3.5-7. They have a negatively charged surface in either sodium acetate buffer (pH 5.2) or pure H2O. The electrostatic interaction between the virus and the liposome or synthetic lipid nanoparticle is the most significant factor driving adsorption. By modifying the charge density of the lipid nanoparticle, e.g. inclusion of neutral lipids into the lipid nanoparticle, it is possible to modulate the interaction between the lipid nanoparticle and the virus, hence modulating the assembly. In one embodiment, the liposome comprises a cationic lipid.

In one embodiment, the liposome of the particle delivery system comprises a CRISPR system component.

In one aspect, the invention provides a delivery system comprising one or more hybrid virus capsid proteins in combination with a lipid particle, wherein the hybrid virus capsid protein comprises at least a portion of a virus capsid protein attached to at least a portion of a non-capsid protein.

In one embodiment, the virus capsid protein of the delivery system is attached to a surface of the lipid particle. When the lipid particle is a bilayer, e.g., a liposome, the lipid particle comprises an exterior hydrophilic surface and an interior hydrophilic surface. In one embodiment, the virus capsid protein is attached to a surface of the lipid particle by an electrostatic interaction or by hydrophobic interaction.

In one embodiment, the particle delivery system has a diameter of 50-1000 nm, preferably 100-1000 nm.

In one embodiment, the delivery system comprises a non-capsid protein or peptide, wherein the non-capsid protein or peptide has a molecular weight of up to a megadalton. In one embodiment, the non-capsid protein or peptide has a molecular weight in the range of 110 to 160 kDa, 160 to 200 kDa, 200 to 250 kDa, 250 to 300 kDa, 300 to 400 kDa, or 400 to 500 kDa.

In one embodiment, the delivery system comprises a non-capsid protein or peptide, wherein the protein or peptide comprises a CRISPR protein or peptide. In one embodiment, the protein or peptide comprises a Cas9, a Cpf1 or a C2c2/Cas13a.

In one embodiment, a weight ratio of hybrid capsid protein to wild-type capsid protein is from 1:10 to 1:1, for example, 1:1, 1:2, 1:3, 1:4, 1:5, 1:6, 1:7, 1:8, 1:9 and 1:10.

In one embodiment, the virus of the delivery system is an Adenoviridae or a Parvoviridae or a Rhabdoviridae or an enveloped virus having a glycoprotein protein. In one embodiment, the virus is an adeno-associated virus (AAV) or an adenovirus or a VSV or a rabies virus. In one embodiment, the virus is a retrovirus or a lentivirus. In one embodiment, the virus is murine leukemia virus (MuMLV).

In one embodiment, the virus capsid protein of the delivery system comprises VP1, VP2 or VP3.

In one embodiment, the virus capsid protein of the delivery system is VP3, and the non-capsid protein is inserted into or tethered or connected to VP3 loop 3 or loop 6.

In one embodiment, the virus of the delivery system is delivered to the interior of a cell.

In one embodiment, the virus capsid protein and the non-capsid protein are capable of dissociating after delivery into a cell.

In one aspect of the delivery system, the virus capsid protein is attached to the non-capsid protein by a linker. In one embodiment, the linker comprises amino acids. In one embodiment, the linker is a chemical linker. In another embodiment, the linker is cleavable or biodegradable. In one embodiment, the linker comprises (GGGGS)1-3, ENLYFQG, or a disulfide.

In one embodiment of the delivery system, each terminus of the non-capsid protein is attached to the capsid protein by a linker moiety.

In one embodiment, the non-capsid protein is attached to the exterior portion of the virus capsid protein. As used herein, “exterior portion” as it refers to a virus capsid protein means the outer surface of the virus capsid protein when it is in a formed virus capsid.

In one embodiment, the non-capsid protein is attached to the interior portion of the capsid protein or is encapsulated within the lipid particle. As used herein, “interior portion” as it refers to a virus capsid protein means the inner surface of the virus capsid protein when it is in a formed virus capsid. In one embodiment, the virus capsid protein and the non-capsid protein are a fusion protein.

In one embodiment, the fusion protein is attached to the surface of the lipid particle.

In one embodiment, the non-capsid protein is attached to the virus capsid protein prior to formation of the capsid.

In one embodiment, the non-capsid protein is attached to the virus capsid protein after formation of the capsid.

In one embodiment, the non-capsid protein comprises a targeting moiety.

In one embodiment, the targeting moiety comprises a receptor ligand.

In an embodiment, the non-capsid protein comprises a tag.

In an embodiment, the non-capsid protein comprises one or more heterologous nuclear localization signals(s) (NLSs).

In an embodiment, the protein or peptide comprises a Type II CRISPR protein or a Type V CRISPR protein.

In an embodiment, the delivery system further comprises guide RNS, optionally complexed with the CRISPR protein.

In an embodiment, the delivery system comprises a protease or nucleic acid molecule(s) encoding a protease that is expressed, whereby the protease cleaves the linker. In certain embodients, there is protease expression, linker cleavage, and dissociation of payload from capsid in the absence of productive virus replication.

In an aspect, the invention provides a delivery system comprising a first hybrid virus capsid protein and a second hybrid virus capsid protein, wherein the first hybrid virus capsid protein comprises a virus capsid protein attached to a first part of a protein, and wherein the second hybrid virus capsid protein comprises a second virus capsid protein attached to a second part of the protein, wherein the first part of the protein and the second part of the protein are capable of associating to form a functional protein.

In an aspect, the invention provides a delivery system comprising a first hybrid virus capsid protein and a second hybrid virus capsid protein, wherein the first hybrid virus capsid protein comprises a virus capsid protein attached to a first part of a CRISPR protein, and wherein the second hybrid virus capsid protein comprises a second virus capsid protein attached to a second part of a CRISPR protein, wherein the first part of the CRISPR protein and the second part of the CRISPR protein are capable of associating to form a functional CRISPR protein.

In an embodiment of the delivery system, the first hybrid virus capsid protein and the second virus capsid protein are on the surface of the same virus particle.

In an embodiment of the delivery system, the first hybrid virus capsule protein is located at the interior of a first virus particle and the second hybrid virus capsid protein is located at the interior of a second virus particle.

In an embodiment of the delivery system, the first part of the protein or CRISPR protein is linked to a first member of a ligand pair, and the second part of the protein or CRISPR protein is linked to a second member of a ligand pair, wherein the first part of the ligand pair binds to the second part of the ligand pair in a cell. In an embodiment, the binding of the first part of the ligand pair to the second part of the ligand pair is inducible.

In an embodiment of the delivery system, either or both of the first part of the protein or CRISPR protein and the second part of the protein or CRISPR protein comprise one or more NLSs.

In an embodiment of the delivery system, either or both of the first part of the protein or CRISPR protein and the second part of the protein or CRISPR protein comprise one or more nuclear export signals (NESs).

In certain embodiments, the virus structural component comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned herein, the teachings herein as to modifying adenovirus and AAV, respectively, can be applied to those viruses without undue experimentation from this disclosure and the knowledge in the art.

In another aspect, the invention provides a non-naturally occurring or engineered CRISPR protein associated with Adeno Associated Virus (AAV), e.g., an AAV comprising a CRISPR protein as a fusion, with or without a linker, to or with an AAV capsid protein such as VP 1, VP2, and/or VP3; and, for shorthand purposes, such a non-naturally occurring or engineered CRISPR protein is herein termed a “AAV-CRISPR protein” More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a CRISPR-protein. Likewise, these can be fusions, with the protein, e.g., large payload protein such as a CRISPR-protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid. Applicants provide AAV capsid -CRISPR protein (e.g., Cas, Cas9, dCas9, Cpf1, Cas13a, Cas13b) fusions and those AAV-capsid CRISPR protein (e.g., Cas, Cas9) fusions can be a recombinant AAV that contains nucleic acid molcule(s) encoding or providing CRISPR-Cas or CRISPR system or complex RNA guide(s), whereby the CRISPR protein (e.g., Cas, Cas9) fusion delivers a CRISPR-Cas or CRISPR system complex (e.g., the CRISPR protein or Cas or Cas9 or Cpf1 is provided by the fusion, e.g., VP1, VP2, pr VP3 fusion, and the guide RNA is provided by the coding of the recombinant virus, whereby in vivo, in a cell, the CRISPR-Cas or CRISPR system is assembled from the nucleic acid molecule(s) of the recombinant providing the guide RNA and the outer surface of the virus providing the CRISPR-Enzyme or Cas or Cas9. Such as complex may herein be termed an “AAV-CRISPR system” or an “AAV—CRISPR-Cas” or “AAV-CRISPR complex” or AAV—CRISPR-Cas complex.” Accordingly, the instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention with discussion herein as to AAV applicable to such other viruses.

In one aspect, the invention provides a non-naturally occurring or engineered composition comprising a CRISPR enzyme which is part of or tethered to a AAV capsid domain, i.e., VP1, VP2, or VP3 domain of Adeno-Associated Virus (AAV) capsid. In some embodiments, part of or tethered to a AAV capsid domain includes associated with associated with a AAV capsid domain. In some embodiments, the CRISPR enzyme may be fused to the AAV capsid domain. In some embodiments, the fusion may be to the N-terminal end of the AAV capsid domain. As such, in some embodiments, the C-terminal end of the CRISPR enzyme is fused to the N-terminal end of the AAV capsid domain. In some embodiments, an NLS and/or a linker (such as a GlySer linker) may be positioned between the C-terminal end of the CRISPR enzyme and the N-terminal end of the AAV capsid domain. In some embodiments, the fusion may be to the C-terminal end of the AAV capsid domain. In some embodiments, this is not preferred due to the fact that the VP1, VP2 and VP3 domains of AAV are alternative splices of the same RNA and so a C-terminal fusion may affect all three domains. In some embodiments, the AAV capsid domain is truncated. In some embodiments, some or all of the AAV capsid domain is removed. In some embodiments, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the CRISPR protein. A branched linker may be used, with the CRISPR protein fused to the end of one of the braches. This allows for some degree of spatial separation between the capsid and the CRISPR protein. In this way, the CRISPR protein is part of (or fused to) the AAV capsid domain.

Alternatively, the CRISPR enzyme may be fused in frame within, i.e. internal to, the AAV capsid domain. Thus in some embodiments, the AAV capsid domain again preferably retains its N-terminal and C-terminal ends. In this case, a linker is preferred, in some embodiments, either at one or both ends of the CRISPR enzyme. In this way, the CRISPR enzyme is again part of (or fused to) the AAV capsid domain. In certain embodiments, the positioning of the CRISPR enzyme is such that the CRISPR enzyme is at the external surface of the viral capsid once formed. In one aspect, the invention provides a non-naturally occurring or engineered composition comprising a CRISPR enzyme associated with a AAV capsid domain of Adeno-Associated Virus (AAV) capsid. Here, associated may mean in some embodiments fused, or in some embodiments bound to, or in some embodiments tethered to. The CRISPR protein may, in some embodiments, be tethered to the VP1, VP2, or VP3 domain. This may be via a connector protein or tethering system such as the biotin-streptavidin system. In one example, a biotinylation sequence (15 amino acids) could therefore be fused to the CRISPR protein. When a fusion of the AAV capsid domain, especially the N-terminus of the AAV AAV capsid domain, with streptavidin is also provided, the two will therefore associate with very high affinity. Thus, in some embodiments, provided is a composition or system comprising a CRISPR protein-biotin fusion and a streptavidin-AAV capsid domain arrangement, such as a fusion. The CRISPR protein-biotin and streptavidin-AAV capsid domain forms a single complex when the two parts are brought together. NLSs may also be incorporated between the CRISPR protein and the biotin; and/or between the streptavidin and the AAV capsid domain.

An alternative tether may be to fuse or otherwise associate the AAV capsid domain to an adaptor protein which binds to or recognizes to a corresponding RNA sequence or motif. In some embodiments, the adaptor is or comprises a binding protein which recognizes and binds (or is bound by) an RNA sequence specific for said binding protein. In some embodiments, a preferred example is the MS2 (see Konermann et al. December 2014, cited infra, incorporated herein by reference) binding protein which recognizes and binds (or is bound by) an RNA sequence specific for the MS2 protein.

With the AAV capsid domain associated with the adaptor protein, the CRISPR protein may, in some embodiments, be tethered to the adaptor protein of the AAV capsid domain. The CRISPR protein may, in some embodiments, be tethered to the adaptor protein of the AAV capsid domain via the CRISPR enzyme being in a complex with a modified guide, see Konermann et al. The modified guide is, in some embodiments, a sgRNA. In some embodiments, the modified guide comprises a distinct RNA sequence; see, e.g., PCT/US 14/70175, incorporated herein by reference.

In some embodiments, distinct RNA sequence is an aptamer. Thus, corresponding aptamer-adaptor protein systems are preferred. One or more functional domains may also be associated with the adaptor protein. An example of a preferred arrangement would be:

[AAV AAV capsid domain−adaptor protein]−[modified guide−CRISPR protein]

In certain embodiments, the positioning of the CRISPR protein is such that the CRISPR protein is at the internal surface of the viral capsid once formed. In one aspect, the invention provides a non-naturally occurring or engineered composition comprising a CRISPR protein associated with an internal surface of an AAV capsid domain. Here again, associated may mean in some embodiments fused, or in some embodiments bound to, or in some embodiments tethered to. The CRISPR protein may, in some embodiments, be tethered to the VP1, VP2, or VP3 domain such that it locates to the internal surface of the viral capsid once formed. This may be via a connector protein or tethering system such as the biotin-streptavidin system as described above.

When the CRISPR protein fusion is designed so as to position the CRISPR protein at the internal surface of the capsid once formed, the CRISPR protein will fill most or all of internal volume of the capsid. Alternatively the CRISPR protein may be modified or divided so as to occupy a less of the capsid internal volume. Accordingly, in certain embodiments, the invention provides a CRISRP protein divided in two portions, one portion comprises in one viral particle or capsid and the second portion comprised in a second viral particle or capsid. In certain embodiments, by splitting the CRISPR protein in two portions, space is made available to link one or more heterologous domains to one or both CRISPR protein portions.

Split CRISPR proteins are set forth herein and in documents incorporated herein by reference in further detail herein. In certain embodiments, each part of a split CRISRP proteins are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In general, according to the invention, CRISPR proteins may preferably split between domains, leaving domains intact. Preferred, non-limiting examples of such CRISPR proteins include, without limitation, Cas9, Cpf1, C2c2, Cas13a, Cas13b, and orthologues. Preferred, non-limiting examples of split points include, with reference to SpCas9: a split position between 202A/203S; a split position between 255F/256D; a split position between 310E/311I; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099.

In some embodiments, any AAV serotype is preferred. In some embodiments, the VP2 domain associated with the CRISPR enzyme is an AAV serotype 2 VP2 domain. In some embodiments, the VP2 domain associated with the CRISPR enzyme is an AAV serotype 8 VP2 domain. The serotype can be a mixed serotype as is known in the art.

The CRISPR enzyme may form part of a CRISPR-Cas system, which further comprises a guide RNA (sgRNA) comprising a guide sequence capable of hybridizing to a target sequence in a genomic locus of interest in a cell. In some embodiments, the functional CRISPR-Cas system binds to the target sequence. In some embodiments, the functional CRISPR-Cas system may edit the genomic locus to alter gene expression. In some embodiments, the functional CRISPR-Cas system may comprise further functional domains.

In some embodiments, the CRISPR enzyme is a Cpf1. In some embodiments, the CRISPR enzyme is an FnCpf1. In some embodiments, the CRISPR enzyme is an AsCpf1, although other orthologs are envisaged. FnCpf1 and AsCpf1 are particularly preferred, in some embodiments.

In some embodiments, the CRISPR enzyme is external to the capsid or virus particle. In the sense that it is not inside the capsid (enveloped or encompassed with the capsid), but is externally exposed so that it can contact the target genomic DNA). In some embodiments, the CRISPR enzyme cleaves both strands of DNA to produce a double strand break (DSB). In some embodiments, the CRISPR enzyme is a nickase. In some embodiments, the CRISPR enzyme is a dual nickase. In some embodiments, the CRISPR enzyme is a deadCpf1. In some general embodiments, the CRISPR enzyme is associated with one or more functional domains. In some more specific embodiments, the CRISPR enzyme is a deadCpf1 and is associated with one or more functional domains. In some embodiments, the CRISPR enzyme comprises a Rec2 or HD2 truncation. In some embodiments, the CRISPR enzyme is associated with the AAV VP2 domain by way of a fusion protein. In some embodiments, the CRISPR enzyme is fused to Destabilization Domain (DD). In other words, the DD may be associated with the CRISPR enzyme by fusion with said CRISPR enzyme. The AAV can then, by way of nucleic acid molecule(s) deliver the stabilizing ligand (or such can be otherwise delivered) In some embodiments, the enzyme may be considered to be a modified CRISPR enzyme, wherein the CRISPR enzyme is fused to at least one destabilization domain (DD) and VP2. In some embodiments, the association may be considered to be a modification of the VP2 domain. Where reference is made herein to a modified VP2 domain, then this will be understood to include any association discussed herein of the VP2 domain and the CRISPR enzyme. In some embodiments, the AAV VP2 domain may be associated (or tethered) to the CRISPR enzyme via a connector protein, for example using a system such as the streptavidin-biotin system. As such, provided is a fusion of a CRISPR enzyme with a connector protein specific for a high affinity ligand for that connector, whereas the AAV VP2 domain is bound to said high affinity ligand. For example, streptavidin may be the connector fused to the CRISPR enzyme, while biotin may be bound to the AAV VP2 domain. Upon co-localization, the streptavidin will bind to the biotin, thus connecting the CRISPR enzyme to the AAV VP2 domain. The reverse arrangement is also possible. In some embodiments, a biotinylation sequence (15 amino acids) could therefore be fused to the AAV VP2 domain, especially the N-terminus of the AAV VP2 domain. A fusion of the CRISPR enzyme with streptavidin is also preferred, in some embodiments. In some embodiments, the biotinylated AAV capsids with streptavidin-CRISPR enzyme are assembled in vitro. This way the AAV capsids should assemble in a straightforward manner and the CRISPR enzyme-streptavidin fusion can be added after assembly of the capsid. In other embodiments a biotinylation sequence (15 amino acids) could therefore be fused to the CRISPR enzyme, together with a fusion of the AAV VP2 domain, especially the N-terminus of the AAV VP2 domain, with streptavidin. For simplicity, a fusion of the CRISPR enzyme and the AAV VP2 domain is preferred in some embodiments. In some embodiments, the fusion may be to the N-terminal end of the CRISPR enzyme. In other words, in some embodiments, the AAV and CRISPR enzyme are associated via fusion. In some embodiments, the AAV and CRISPR enzyme are associated via fusion including a linker. Suitable linkers are discussed herein, but include Gly Ser linkers. Fusion to the N-term of AAV VP2 domain is preferred, in some embodiments. In some embodiments, the CRISPR enzyme comprises at least one Nuclear Localization Signal (NLS). In an aspect, the present invention provides a polynucleotide encoding the present CRISPR enzyme and associated AAV VP2 domain.

Viral delivery vectors, for example modified viral delivery vectors, are hereby provided. While the AAV may advantageously be a vehicle for providing RNA of the CRISPR-Cas Complex or CRISPR system, another vector may also deliver that RNA, and such other vectors are also herein discussed. In one aspect, the invention provides a non-naturally occurring modified AAV having a VP2-CRISPR enzyme capsid protein, wherein the CRISPR enzyme is part of or tethered to the VP2 domain. In some preferred embodiments, the CRISPR enzyme is fused to the VP2 domain so that, in another aspect, the invention provides a non-naturally occurring modified AAV having a VP2-CRISPR enzyme fusion capsid protein. The following embodiments apply equally to either modified AAV aspect, unless otherwise apparent. Thus, reference herein to a VP2-CRISPR enzyme capsid protein may also include a VP2-CRISPR enzyme fusion capsid protein. In some embodiments, the VP2-CRISPR enzyme capsid protein further comprises a linker. In some embodiments, the VP2-CRISPR enzyme capsid protein further comprises a linker, whereby the VP2-CRISPR enzyme is distanced from the remainder of the AAV. In some embodiments, the VP2-CRISPR enzyme capsid protein further comprises at least one protein complex, e.g., CRISPR complex, such as CRISPR-Cpf1 complex guide RNA that targets a particular DNA, TALE, etc. A CRISPR complex, such as CRISPR-Cas system comprising the VP2-CRISPR enzyme capsid protein and at least one CRISPR complex, such as CRISPR-Cpf1 complex guide RNA that targets a particular DNA, is also provided in one aspect. In general, in some embodiments, the AAV further comprises a repair template. It will be appreciated that comprises here may mean encompassed thin the viral capsid or that the virus encodes the comprised protein. In some embodiments, one or more, preferably two or more guide RNAs, may be comprised/encompassed within the AAV vector. Two may be preferred, in some embodiments, as it allows for multiplexing or dual nickase approaches. Particularly for multiplexing, two or more guides may be used. In fact, in some embodiments, three or more, four or more, five or more, or even six or more guide RNAs may be comprised/encompassed within the AAV. More space has been freed up within the AAV by virtue of the fact that the AAV no longer needs to comprise/encompass the CRISPR enzyme. In each of these instances, a repair template may also be provided comprised/encompassed within the AAV. In some embodiments, the repair template corresponds to or includes the DNA target.

In a further aspect, the present invention provides compositions comprising the CRISPR enzyme and associated AAV VP2 domain or the polynucleotides or vectors described herein. Also provides are CRISPR-Cas systems comprising guide RNAs.

Also provided is a method of treating a subject in need thereof, comprising inducing gene editing by transforming the subject with the polynucleotide encoding the system or any of the present vectors. A suitable repair template may also be provided, for example delivered by a vector comprising said repair template. In some embodiments, a single vector provides the CRISPR enzyme through (association with the viral capsid) and at least one of: guide RNA; and/or a repair template. Also provided is a method of treating a subject in need thereof, comprising inducing transcriptional activation or repression by transforming the subject with the polynucleotide encoding the present system or any of the present vectors, wherein said polynucleotide or vector encodes or comprises the catalytically inactive CRISPR enzyme and one or more associated functional domains. Compositions comprising the present system for use in said method of treatment are also provided. A kit of parts may be provided including such compositions. Use of the present system in the manufacture of a medicament for such methods of treatment are also provided.

Also provided is a pharmaceutical composition comprising the CRISPR enzyme which is part of or tethered to a VP2 domain of Adeno-Associated Virus (AAV) capsid; or the non-naturally occurring modified AAV; or a polynucleotide encoding them.

Also provided is a complex of the CRISPR enzyme with a guideRNA, such as sgRNA. The complex may further include the target DNA.

A split CRISPR enzyme, most preferably Cpf1, approach may be used. The so-called ‘split Cpf1’ approach Split Cpf1 allows for the following. The Cpf1 is split into two pieces and each of these are fused to one half of a dimer. Upon dimerization, the two parts of the Cpf1 are brought together and the reconstituted Cpf1 has been shown to be functional. Thus, one part of the split Cpf1 may be associated with one VP2 domain and second part of the split Cpf1 may be associated with another VP2 domain. The two VP2 domains may be in the same or different capsid. In other words, the split parts of the Cpf1 could be on the same virus particle or on different virus particles.

In some embodiments, one or more functional domains may be associated with or tethered to CRISPR enzyme and/or may be associated with or tethered to modified guides via adaptor proteins. These can be used irrespective of the fact that the CRISPR enzyme may also be tethered to a virus outer protein or capsid or envelope, such as a VP2 domain or a capsid, via modified guides with aptamer RAN sequences that recognize correspond adaptor proteins.

In some embodiments, one or more functional domains comprise a transcriptional activator, repressor, a recombinase, a transposase, a histone remodeler, a demethylase, a DNA methyltransferase, a cryptochrome, a light inducible/controllable domain, a chemically inducible/controllable domain, an epigenetic modifying domain, or a combination thereof. Advantageously, the functional domain comprises an activator, repressor or nuclease.

In some embodiments, a functional domain can have methylase activity, demethylase activity, transcription activation activity, transcription repression activity, transcription release factor activity, histone modification activity, RNA cleavage activity or nucleic acid binding activity, or activity that a domain identified herein has.

Examples of activators include P65, a tetramer of the herpes simplex activation domain VP 16, termed VP64, optimized use of VP64 for activation through modification of both the sgRNA design and addition of additional helper molecules, MS2, P65 and HSF lin the system called the synergistic activation mediator (SAM) (Konermann et al, “Genome-scale transcriptional activation by an engineered CRISPR-Cas9 complex,” Nature 517(7536):583-8 (2015)); and examples of repressors include the KRAB (Kruppel-associated box) domain of Koxl or SID domain (e.g. SID4X); and an example of a nuclease or nuclease domain suitable for a functional domain comprises Fok1.

Suitable functional domains for use in practice of the invention, such as activators, repressors or nucleases are also discussed in documents incorporated herein by reference, including the patents and patent publications herein-cited and incorporated herein by reference regarding general information on CRISPR-Cas Systems.

In some embodiments, the CRISPR enzyme comprises or consists essentially of or consists of a localization signal as, or as part of, the linker between the CRISPR enzyme and the AAV capsid, e.g., VP2. HA or Flag tags are also within the ambit of the invention as linkers as well as Glycine Serine linkers as short as GS up to (GGGGS)3. In this regard it is mentioned that tags that can be used in embodiments of the invention include affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; fluorescence tags, such as GFP and mCherry; protein tags that may allow specific enzymatic modification (such as biotinylation by biotin ligase) or chemical modification (such as reaction with F1AsH-EDT2 for fluorescence imaging).

Also provided is a method of treating a subject, e.g, a subject in need thereof, comprising inducing gene editing by transforming the subject with the AAV-CRISPR enzyme advantageously encoding and expressing in vivo the remaining portions of the CRISPR system (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the AAV-CRISPR enzyme advantageously encoding and expressing in vivo the remaining portions of the CRISPR system (e.g., RNA, guides); advantageously in some embodiments the CRISPR enzyme is a catalytically inactive CRISPR enzyme and comprises one or more associated functional domains. Where any treatment is occurring ex vivo, for example in a cell culture, then it will be appreciated that the term ‘subject’ may be replaced by the phrase “cell or cell culture.”

Compositions comprising the present system for use in said method of treatment are also provided. A kit of parts may be provided including such compositions. Use of the present system in the manufacture of a medicament for such methods of treatment are also provided. Use of the present system in screening is also provided by the present invention, e.g., gain of function screens. Cells which are artificially forced to overexpress a gene are be able to down regulate the gene over time (re-establishing equilibrium) e.g. by negative feedback loops. By the time the screen starts the unregulated gene might be reduced again.

In one aspect, the invention provides an engineered, non-naturally occurring CRISPR-Cas system comprising a AAV-Cas protein and a guide RNA that targets a DNA molecule encoding a gene product in a cell, whereby the guide RNA targets the DNA molecule encoding the gene product and the Cas protein cleaves the DNA molecule encoding the gene product, whereby expression of the gene product is altered; and, wherein the Cas protein and the guide RNA do not naturally occur together. The invention comprehends the guide RNA comprising a guide sequence fused to a tracr sequence. In an embodiment of the invention the Cas protein is a type II CRISPR-Cas protein and in a preferred embodiment the Cas protein is a Cpf1 protein. The invention further comprehends the coding for the Cas protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides an engineered, non-naturally occurring vector system comprising one or more vectors comprising a first regulatory element operably linked to a CRISPR-Cas system guide RNA that targets a DNA molecule encoding a gene product and a AAV-Cas protein. The components may be located on same or different vectors of the system, or may be the same vector whereby the AAV-Cas protein also delivers the RNA of the CRISPR system. The guide RNA targets the DNA molecule encoding the gene product in a cell and the AAV-Cas protein may cleaves the DNA molecule encoding the gene product (it may cleave one or both strands or have substantially no nuclease activity), whereby expression of the gene product is altered; and, wherein the AAV-Cas protein and the guide RNA do not naturally occur together. The invention comprehends the guide RNA comprising a guide sequence fused to a tracr sequence. In an embodiment of the invention the AAV-Cas protein is a type II AAV—CRISPR-Cas protein and in a preferred embodiment the AAV-Cas protein is a AAV-Cpf1 protein. The invention further comprehends the coding for the AAV-Cas protein being codon optimized for expression in a eukaryotic cell. In a preferred embodiment the eukaryotic cell is a mammalian cell and in a more preferred embodiment the mammalian cell is a human cell. In a further embodiment of the invention, the expression of the gene product is decreased.

In another aspect, the invention provides a method of expressing an effector protein and guide RNA in a cell comprising introducing the vector according any of the vector delivery systems disclosed herein. In an embodiment of the vector for delivering an effector protein, the minimnal promoter is the Mecp2 promoter, tRNA promoter, or U6. In a further embodiment, the minimal promoter is tissue specific.

The one or more polynucleotide molecules may be comprised within one or more vectors. The invention comprehends such polynucleotide molecule(s), for instance such polynucleotide molecules operably configured to express the protein and/or the nucleic acid component(s), as well as such vector(s).

In one aspect, the invention provides a vector system comprising one or more vectors. In some embodiments, the system comprises: (a) a first regulatory element operably linked to a tracr mate sequence and one or more insertion sites for inserting one or more guide sequences upstream of the tracr mate sequence, wherein when expressed, the guide sequence directs sequence-specific binding of a AAV-CRISPR complex to a target sequence in a eukaryotic cell, wherein the CRISPR complex comprises a AAV-CRISPR enzyme complexed with (1) the guide sequence that is hybridized to the target sequence, and (2) the tracr mate sequence that is hybridized to the tracr sequence; and (b) said AAV-CRISPR enzyme comprising at least one nuclear localization sequence and/or at least one NES; wherein components (a) and (b) are located on or in the same or different vectors of the system. In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of a AAV-CRISPR complex to a different target sequence in a eukaryotic cell. In some embodiments, the system comprises the tracr sequence under the control of a third regulatory element, such as a polymerase III promoter. In some embodiments, the tracr sequence exhibits at least 50%, 60%, 70%, 80%, 90%, 95%, or 99% of sequence complementarity along the length of the tracr mate sequence when optimally aligned. Determining optimal alignment is within the purview of one of skill in the art. For example, there are publically and commercially available alignment algorithms and programs such as, but not limited to, ClustalW, Smith-Waterman in matlab, Bowtie, Geneious, Biopython and SeqMan. In some embodiments, the AAV-CRISPR complex comprises one or more nuclear localization sequences of sufficient strength to drive accumulation of said CRISPR complex in a detectable amount in the nucleus of a eukaryotic cell. Without wishing to be bound by theory, it is believed that a nuclear localization sequence is not necessary for AAV-CRISPR complex activity in eukaryotes, but that including such sequences enhances activity of the system, especially as to targeting nucleic acid molecules in the nucleus and/or having molecules exit the nucleus. In some embodiments, the AAV-CRISPR enzyme is a type II AAV-CRISPR system enzyme. In some embodiments, the AAV-CRISPR enzyme is a AAV-Cpf1 enzyme. In some embodiments, the AAV-Cpf1 enzyme is derived from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae (e.g., a Cpf1 of one of these organisms modified to have or be associated with at least one AAV), and may include further mutations or alterations or be a chimeric Cpf1. The enzyme may be a AAV-Cpf1 homolog or ortholog. In some embodiments, the AAV-CRISPR enzyme is codon-optimized for expression in a eukaryotic cell. In some embodiments, the AAV-CRISPR enzyme directs cleavage of one or two strands at the location of the target sequence. In some embodiments, the AAV-CRISPR enzyme lacks DNA strand cleavage activity. In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Again, the RNA of the CRISPR System, while advantageously delivered via the AAV-CRISPR enzyme can also be delivered separately, e.g. via a separate vector.

In one aspect, the invention provides an AAV-CRISPR enzyme comprising one or more nuclear localization sequences and/or NES. In some embodiments, said AAV-CRISPR enzyme includes a regulatory element that drives transcription of component(s) of the CRISPR system (e.g., RNA, such as guide RNA and/or HR template nucleic acid molecule) in a eukaryotic cell such that said AAV-CRISPR enzyme delivers the CRISPR system accumulates in a detectable amount in the nucleus of the eukaryotic cell and/or is exported from the nucleus. In some embodiments, the regulatory element is a polymerase II promoter. In some embodiments, the AAV-CRISPR enzyme is a type II AAV-CRISPR system enzyme. In some embodiments, the AAV-CRISPR enzyme is a AAV-Cpf1 enzyme. In some embodiments, the AAV-Cpf1 enzyme is derived from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae (e.g., Cpf1 modified to have or be associated with at least one AAV), and may include further alteration or mutation of the Cpf1, and can be a chimeric Cpf1. In some embodiments, the AAV-CRISPR enzyme is codon-optimized for expression in a eukaryotic cell. In some embodiments, the AAV-CRISPR enzyme directs cleavage of one or two strands at the location of the target sequence. In some embodiments, the AAV-CRISPR enzyme lacks or substantially DNA strand cleavage activity (e.g., no more than 5% nuclease activity as compared with a wild type enzyme or enzyme not having the mutation or alteration that decreases nuclease activity).

In one aspect, the invention provides a AAV-CRISPR enzyme comprising one or more nuclear localization sequences of sufficient strength to drive accumulation of said AAV-CRISPR enzyme in a detectable amount in the nucleus of a eukaryotic cell. In some embodiments, the AAV-CRISPR enzyme is a type II AAV-CRISPR system enzyme. In some embodiments, the AAV-CRISPR enzyme is a AAV-Cpf1 enzyme. In some embodiments, the AAV-Cpf1 enzyme is derived from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae (e.g., Cpf1 modified to have or be associated with at least one AAV), and may include further alteration or mutation of the Cpf1, and can be a chimeric Cpf1. In some embodiments, the AAV-CRISPR enzyme is codon-optimized for expression in a eukaryotic cell. In some embodiments, the AAV-CRISPR enzyme directs cleavage of one or two strands at the location of the target sequence. In some embodiments, the AAV-CRISPR enzyme lacks or substantially DNA strand cleavage activity (e.g., no more than 5% nuclease activity as compared with a wild type enzyme or enzyme not having the mutation or alteration that decreases nuclease activity).

In one aspect, the invention provides a eukaryotic host cell comprising (a) a first regulatory element operably linked to a tracr mate sequence and one or more insertion sites for inserting one or more guide sequences upstream of the tracr mate sequence, wherein when expressed, the guide sequence directs sequence-specific binding of a AAV-CRISPR complex to a target sequence in a eukaryotic cell, wherein the AAV-CRISPR complex comprises a AAV-CRISPR enzyme complexed with (1) the guide sequence that is hybridized to the target sequence, and (2) the tracr mate sequence that is hybridized to the tracr sequence; and/or (b) a said AAV-CRISPR enzyme optionally comprising at least one nuclear localization sequence and/or NES. In some embodiments, the host cell comprises components (a) and (b). In some embodiments, component (a), component (b), or components (a) and (b) are stably integrated into a genome of the host eukaryotic cell. In some embodiments, component (b) includes or contains component (a). In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of a AAV-CRISPR complex to a different target sequence in a eukaryotic cell. In some embodiments, the eukaryotic host cell further comprises a third regulatory element, such as a polymerase III promoter, operably linked to said tracr sequence. In some embodiments, the tracr sequence exhibits at least 50%, 60%, 70%, 80%, 90%, 95%, or 99% of sequence complementarity along the length of the tracr mate sequence when optimally aligned. In some embodiments, the AAV-CRISPR enzyme comprises one or more nuclear localization sequences and/or nuclear export sequences of sufficient strength to drive accumulation of said CRISPR enzyme in a detectable amount in of the nucleus of a eukaryotic cell. In some embodiments, the AAV-CRISPR enzyme is a type II CRISPR system enzyme. In some embodiments, the CRISPR enzyme is a Cpf1 enzyme. In some embodiments, the AAV-Cpf1 enzyme is derived from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae (e.g., Cpf1 modified to have or be associated with at least one AAV), and may include further alteration or mutation of the Cpf1, and can be a chimeric Cpf1. In some embodiments, the AAV-CRISPR enzyme is codon-optimized for expression in a eukaryotic cell. In some embodiments, the AAV-CRISPR enzyme directs cleavage of one or two strands at the location of the target sequence. In some embodiments, the AAV-CRISPR enzyme lacks or substantially DNA strand cleavage activity (e.g., no more than 5% nuclease activity as compared with a wild type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal; for example a mammal. Also, the organism may be an arthropod such as an insect. The organism also may be a plant. Further, the organism may be a fungus. Advantageoulsy the organism is a host of AAV.

In one aspect, the invention provides a kit comprising one or more of the components described herein. In some embodiments, the kit comprises a vector system and instructions for using the kit. In some embodiments, the vector system comprises (a) a first regulatory element operably linked to a tracr mate sequence and one or more insertion sites for inserting one or more guide sequences upstream of the tracr mate sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) the tracr mate sequence that is hybridized to the tracr sequence; and/or (b) said AAV-CRISPR enzyme optionally comprising a nuclear localization sequence. In some embodiments, the kit comprises components (a) and (b) located on or in the same or different vectors of the system, e.g., (a) can be contained in (b). In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the system further comprises a third regulatory element, such as a polymerase III promoter, operably linked to said tracr sequence. In some embodiments, the tracr sequence exhibits at least 50%, 60%, 70%, 80%, 90%, 95%, or 99% of sequence complementarity along the length of the tracr mate sequence when optimally aligned. In some embodiments, the CRISPR enzyme comprises one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cpf1 enzyme. In some embodiments, the Cpf1 enzyme is derived from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae (e.g., Cpf1 modified to have or be associated with at least one AAV), and may include further alteration or mutation of the Cpf1, and can be a chimeric Cpf1. In some embodiments, the coding for the AAV-CRISPR enzyme is codon-optimized for expression in a eukaryotic cell. In some embodiments, the AAV-CRISPR enzyme directs cleavage of one or two strands at the location of the target sequence. In some embodiments, the AAV-CRISPR enzyme lacks or substantially DNA strand cleavage activity (e.g., no more than 5% nuclease activity as compared with a wild type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length.

In one aspect, the invention provides a method of modifying a target polynucleotide in a eukaryotic cell. In some embodiments, the method comprises allowing a AAV-CRISPR complex to bind to the target polynucleotide, e.g., to effect cleavage of said target polynucleotide, thereby modifying the target polynucleotide, wherein the AAV-CRISPR complex comprises a AAV-CRISPR enzyme complexed with a guide sequence hybridized to a target sequence within said target polynucleotide, wherein said guide sequence is linked to a tracr mate sequence which in turn hybridizes to a tracr sequence. In some embodiments, said cleavage comprises cleaving one or two strands at the location of the target sequence by said AAV-CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein one or more vectors comprise the AAV-CRISPR enzyme and one or more vectors drive expression of one or more of: the guide sequence linked to the tracr mate sequence, and the tracr sequence. In some embodiments, said AAV-CRISPR enzyme drive expression of one or more of: the guide sequence linked to the tracr mate sequence, and the tracr sequence. In some embodiments such AAV-CRISPR enzyme are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject.

In one aspect, the invention provides a method of modifying expression of a polynucleotide in a eukaryotic cell. In some embodiments, the method comprises allowing a AAV-CRISPR complex to bind to the polynucleotide such that said binding results in increased or decreased expression of said polynucleotide; wherein the AAV-CRISPR complex comprises a AAV-CRISPR enzyme complexed with a guide sequence hybridized to a target sequence within said polynucleotide, wherein said guide sequence is linked to a tracr mate sequence which in turn hybridizes to a tracr sequence. In some embodiments, the method further comprises delivering one or more vectors to said eukaryotic cells, wherein the one or more vectors are the AAV-CRISPR enzyme and/or drive expression of one or more of: the guide sequence linked to the tracr mate sequence, and the tracr sequence.

In one aspect, the invention provides a method of generating a model eukaryotic cell comprising a mutated disease gene. In some embodiments, a disease gene is any gene associated an increase in the risk of having or developing a disease. In some embodiments, the method comprises (a) introducing one or more vectors into a eukaryotic cell, wherein the one or more vectors comprise the AAV-CRISPR enzyme and/or drive expression of one or more of: a guide sequence linked to a tracr mate sequence, and a tracr sequence; and (b) allowing a AAV-CRISPR complex to bind to a target polynucleotide, e.g., to effect cleavage of the target polynucleotide within said disease gene, wherein the AAV-CRISPR complex comprises the AAV-CRISPR enzyme complexed with (1) the guide sequence that is hybridized to the target sequence within the target polynucleotide, and (2) the tracr mate sequence that is hybridized to the tracr sequence, thereby generating a model eukaryotic cell comprising a mutated disease gene. Thus, in some embodiments the AAV-CRISPR enzyme contains nucleic acid molecules for and drives expression of one or more of: a guide sequence linked to a tracr mate sequence, and a tracr sequence and/or a Homologous Recombination template and/or a stabilizing ligand if the CRISPR enzyme has a destabilization domain. In some embodiments, said cleavage comprises cleaving one or two strands at the location of the target sequence by said AAV-CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the invention provides a method for developing a biologically active agent that modulates a cell signaling event associated with a disease gene. In some embodiments, a disease gene is any gene associated an increase in the risk of having or developing a disease. In some embodiments, the method comprises (a) contacting a test compound with a model cell of any one of the described embodiments; and (b) detecting a change in a readout that is indicative of a reduction or an augmentation of a cell signaling event associated with said mutation in said disease gene, thereby developing said biologically active agent that modulates said cell signaling event associated with said disease gene.

In one aspect, the invention provides a recombinant polynucleotide comprising a guide sequence upstream of a tracr mate sequence, wherein the guide sequence when expressed directs sequence-specific binding of a AAV-CRISPR complex to a corresponding target sequence present in a eukaryotic cell. The polynucleotide can be carried within and expressed in vivo from the AAV-CRISPR enzyme. In some embodiments, the target sequence is a viral sequence present in a eukaryotic cell. In some embodiments, the target sequence is a proto-oncogene or an oncogene.

In one aspect the invention provides for a method of selecting one or more cell(s) by introducing one or more mutations in a gene in the one or more cell (s), the method comprising: introducing one or more vectors into the cell (s), wherein the one or more vectors comprise a AAV-CRISPR enzyme and/or drive expression of one or more of: a guide sequence linked to a tracr mate sequence, a tracr sequence, and an editing template; wherein, for example that which is being expressed is within and expressed in vivo by the AAV-CRISPR enzyme and/or the editing template comprises the one or more mutations that abolish AAV-CRISPR enzyme cleavage; allowing homologous recombination of the editing template with the target polynucleotide in the cell(s) to be selected; allowing a CRISPR complex to bind to a target polynucleotide to effect cleavage of the target polynucleotide within said gene, wherein the AAV-CRISPR complex comprises the AAV-CRISPR enzyme complexed with (1) the guide sequence that is hybridized to the target sequence within the target polynucleotide, and (2) the tracr mate sequence that is hybridized to the tracr sequence, wherein binding of the AAV-CRISPR complex to the target polynucleotide induces cell death, thereby allowing one or more cell(s) in which one or more mutations have been introduced to be selected. In a preferred embodiment, the AAV-CRISPR enzyme is AAV-Cpf1. In another aspect of the invention the cell to be selected may be a eukaryotic cell. Aspects of the invention allow for selection of specific cells without requiring a selection marker or a two-step process that may include a counter-selection system. The cell(s) may be prokaryotic or eukaryotic cells.

With respect to mutations of the AAV-CRISPR enzyme, mutations may be made at any or all residues corresponding to positions 908, 993, and 1263 with reference to amino acid position numbering of AsCpf1 (which may be ascertained for instance by standard sequence comparison tools), or 917 and 1006 with reference to amino acid numbering of FnCpf1, or 832, 925, 947, 1180 with reference to amino acid position numbering of LbCpf1. In particular, any or all of the following mutations are preferred in AsCpf1: D908A, E993A, and D1263; in FnCpf1: D917A and H1006A; in LbCpf1: D832A, E925A, D947A, and D1180A; as well as conservative substitution for any of the replacement amino acids is also envisaged. In an aspect the invention provides as to any or each or all embodiments herein-discussed wherein the AAV-CRISPR enzyme comprises at least one or more, or at least two or more mutations, wherein the at least one or more mutation or the at least two or more mutations is as to D908, E993, or D1263 according to AsCpf1 protein, e.g., D908A, E993A, or D1263 as to AsCpf1, or D917 or H1006 according to FnCpf1, e.g., D917A or H1006A as to FnCpf1, or D832, E925, D947, or D1180 according to LbCpf1, e.g., D832A, E925A, D947A, or D1180A as to LbCpf1, or any corresponding mutation(s) in a Cpf1 of an ortholog to As or Fn or Lb, or the CRISPR enzyme comprises at least one mutation wherein at least D908A, E993A, or D1263 as to AsCpf1 or D917A or H1006A as to FnCpf1 or D832A, E925A, D947A, or D1180A as to LbCpf1 is mutated; or any corresponding mutation(s) in a Cpf1 of an ortholog to As protein or Fn protein or Lb protein.

Aspects of the invention encompass a non-naturally occurring or engineered composition that may comprise a guide RNA (sgRNA) comprising a guide sequence capable of hybridizing to a target sequence in a genomic locus of interest in a cell and a AAV-CRISPR enzyme that may comprise at least one or more nuclear localization sequences, wherein the AAV-CRISPR enzyme comprises one or two or more mutations, such that the enzyme has altered or diminished nuclease activity compared with the wild type enzyme, wherein at least one loop of the sgRNA is modified by the insertion of distinct RNA sequence(s) that bind to one or more adaptor proteins, and wherein the adaptor protein further recruits one or more heterologous functional domains. In an embodiment of the invention the AAV-CRISPR enzyme comprises one or two or more mutations in a residue selected from the group comprising, consisting essentially of, or consisting of D908, E993, or D1263 according to AsCpf1 protein; D917 or H1006 according to FnCpf1; or D832, E925, D947, or D1180 according to LbCpf1. In a further embodiment the AAV-CRISPR enzyme comprises one or two or more mutations selected from the group comprising D908A, E993A, or D1263 as to AsCpf1; D917A or H1006A as to FnCpf1; or D832A, E925A, D947A, or D1180A as to LbCpf1. In another embodiment, the functional domain comprise, consist essentially of a transcriptional activation domain, e.g., VP64. In another embodiment, the functional domain comprise, consist essentially of a transcriptional repressor domain, e.g., KRAB domain, SID domain or a SID4X domain. In embodiments of the invention, the one or more heterologous functional domains have one or more activities selected from the group comprising, consisting essentially of, or consisting of methylase activity, demethylase activity, transcription activation activity, transcription repression activity, transcription release factor activity, histone modification activity, RNA cleavage activity and nucleic acid binding activity. In further embodiments of the invention the cell is a eukaryotic cell or a mammalian cell or a human cell. In further embodiments, the adaptor protein is selected from the group comprising, consisting essentially of, or consisting of MS2, PP7, QP, F2, GA, fr, JP501, M12, R17, BZ13, JP34, JP500, KU1, M11, MX1, TW18, VK, SP, FI, ID2, NL95, TW19, AP205, 4Cb5, Cb8r, 4Cbl2r, 4Cb23r, 7s, PRR1. In another embodiment, the at least one loop of the sgRNA is tetraloop and/or loop2. An aspect of the invention encompasses methods of modifying a genomic locus of interest to change gene expression in a cell by introducing into the cell any of the compositions described herein. An aspect of the invention is that the above elements are comprised in a single composition or comprised in individual compositions, e.g., the AAV-CRISPR enzyme delivers the enzyme as discussed as well as the guide. These compositions may advantageously be applied to a host to elicit a functional effect on the genomic level. In general, the sgRNA are modified in a manner that provides specific binding sites (e.g., aptamers) for adapter proteins comprising one or more functional domains (e.g., via fusion protein) to bind to. The modified sgRNA are modified such that once the sgRNA forms a AAV-CRISPR complex (i.e. AAV-CRISPR enzyme binding to sgRNA and target) the adapter proteins bind and, the functional domain on the adapter protein is positioned in a spatial orientation which is advantageous for the attributed function to be effective. For example, if the functional domain comprise, consist essentially of a transcription activator (e.g., VP64 or p65), the transcription activator is placed in a spatial orientation which allows it to affect the transcription of the target. Likewise, a transcription repressor will be advantageously positioned to affect the transcription of the target and a nuclease (e.g., Fok1) will be advantageously positioned to cleave or partially cleave the target. Again, the AAV-CRISPR enzyme can deliver both the enzyme and the modified guide. The skilled person will understand that modifications to the sgRNA which allow for binding of the adapter+functional domain but not proper positioning of the adapter+functional domain (e.g., due to steric hindrance within the three dimensional structure of the CRISPR complex) are modifications which are not intended. The one or more modified sgRNA may be modified at the tetra loop, the stem loop 1, stem loop 2, or stm loop 3, as described herein, preferably at either the tetra loop or stem loop 2, and most preferably at both the tetra loop and stem loop 2.

As explained herein the functional domains may be, for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). In some cases it is advantageous that additionally at least one NLS is provided. In some instances, it is advantageous to position the NLS at the N terminus. When more than one functional domain is included, the functional domains may be the same or different.

The sgRNA may be designed to include multiple binding recognition sites (e.g., aptamers) specific to the same or different adapter protein. The sgRNA may be designed to bind to the promoter region −1000-+1 nucleic acids upstream of the transcription start site (i.e. TSS), preferably −200 nucleic acids. This positioning improves functional domains which affect gene activation (e.g., transcription activators) or gene inhibition (e.g., transcription repressors). The modified sgRNA may be one or more modified sgRNAs targeted to one or more target loci (e.g., at least 1 sgRNA, at least 2 sgRNA, at least 5 sgRNA, at least 10 sgRNA, at least 20 sgRNA, at least 30 sg RNA, at least 50 sgRNA) comprised in a composition.

Further, the AAV-CRISPR enzyme with diminished nuclease activity is most effective when the nuclease activity is inactivated (e.g., nuclease inactivation of at least 70%, at least 80%, at least 90%, at least 95%, at least 97%, or 100% as compared with the wild type enzyme; or to put in another way, a AAV-Cpf1 enzyme or AAV-CRISPR enzyme having advantageously about 0% of the nuclease activity of the non-mutated or wild type Cpf1 enzyme or CRISPR enzyme, or no more than about 3% or about 5% or about 10% of the nuclease activity of the non-mutated or wild type Cpf1 enzyme or CRISPR enzyme). This is possible by introducing mutations into the RuvC and HNH nuclease domains of the AsCpf1 and orthologs thereof. For example utilizing mutations in a residue selected from the group comprising, consisting essentially of, or consisting of D908, E993, or D1263 according to AsCpf1 protein; D917 or H1006 according to FnCpf1; or D832, E925, D947, or D1180 according to LbCpf1, and more preferably introducing one or more of the mutations selected from the group comprising, consisting essentially of, or consisting of D908A, E993A, or D1263 as to AsCpf1; D917A or H1006A as to FnCpf1; or D832A, E925A, D947A, or D1180A as to LbCpf1. The inactivated CRISPR enzyme may have associated (e.g., via fusion protein) one or more functional domains, e.g., at least one destabilizing domain; or, for instance like those as described herein for the modified sgRNA adaptor proteins, including for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF1, MyoD1. In the event that Fok1 is provided, it is advantageous that multiple Fok1 functional domains are provided to allow for a functional dimer and that sgRNAs are designed to provide proper spacing for functional use (Fok1) as specifically described in Tsai et al. Nature Biotechnology, Vol. 32, Number 6, June 2014). The adaptor protein may utilize known linkers to attach such functional domains. In some cases it is advantageous that additionally at least one NLS is provided. In some instances, it is advantageous to position the NLS at the N terminus. When more than one functional domain is included, the functional domains may be the same or different. In general, the positioning of the one or more functional domain on the inactivated AAV-CRISPR enzyme is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., Fok1) will be advantageously positioned to cleave or partially cleave the target. This may include positions other than the N-/C-terminus of the AAV-CRISPR enzyme. Positioning the functional domain in the Recl domain, the Rec2 domain, the HNH domain, or the PI domain of the AsCpf1 protein or any ortholog corresponding to these domains is advantageous; and again, it is mentioned that the functional domain can be a DD. Positioning of the functional domains to the Recl domain or the Rec2 domain, of the AsCpf1 protein or any ortholog corresponding to these domains, in some instances may be preferred. Fok1 functional domain may be attached at the N terminus. When more than one functional domain is included, the functional domains may be the same or different.

An adaptor protein may be any number of proteins that binds to an aptamer or recognition site introduced into the modified sgRNA and which allows proper positioning of one or more functional domains, once the sgRNA has been incorporated into the AAV-CRISPR complex, to affect the target with the attributed function. As explained in detail in this application such may be coat proteins, preferably bacteriophage coat proteins. The functional domains associated with such adaptor proteins (e.g., in the form of fusion protein) may include, for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF 1, MyoD1. In the event that the functional domain is a transcription activator or transcription repressor it is advantageous that additionally at least an NLS is provided and preferably at the N terminus. When more than one functional domain is included, the functional domains may be the same or different. The adaptor protein may utilize known linkers to attach such functional domains. Such linkers may be used to associate the AAV (e.g., capsid or VP2) with the CRISPR enzyme or have the CRISPR enzyme comprise the AAV (or vice versa).

Thus, sgRNA, e.g., modified sgRNA, the inactivated AAV-CRISPR enzyme (with or without functional domains), and the binding protein with one or more functional domains, may each individually be comprised in a composition and administered to a host individually or collectively. Alternatively, these components may be provided in a single composition for administration to a host, e.g., the AAV-CRISPR enzyme can deliver the RNA or guide or sgRNA or modified sgRNA and/or other components of the CRISPR system. Administration to a host may be performed via viral vectors, advantageously using the AAV-CRISPR enzyme as the delivery vehicle, although other vehicles can be used to deliver components other than the enzyme of the CRISPR system, and such viral vectors can be, for example, lentiviral vector, adenoviral vector, AAV vector. Several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

In an aspect, the invention provides a particle delivery system or the delivery system or the virus particle of any one of any one of the above embodiments or the cell of any one of the above embodiments for use in medicine or in therapy; or for use in a method of modifying an organism or a non-human organism by manipulation of a target sequence in a genomic locus associated with a disease or disorder; or for use in a method of treating or inhibiting a condition caused by one or more mutations in a genetic locus associated with a disease in a eukaryotic organism or a non-human organism; or for use in in vitro, ex vivo or in vivo gene or genome editing; or for use in in vitro, ex vivo or in vivo gene therapy.

In an aspect, the invention provides a pharmaceutical composition comprising the particle delivery system or the delivery system or the virus particle of any one of the above embodiment or the cell of any one of the above embodiment.

### Pharmaceuticals

Another aspect of the invention provides a composition, pharmaceutical composition or vaccine comprising the barrier cell, for instance the intestinal epithelial cells, intestinal epithelial stem cells, or intestinal immune cells or populations thereof, as taught herein.

A “pharmaceutical composition” refers to a composition that usually contains an excipient, such as a pharmaceutically acceptable carrier that is conventional in the art and that is suitable for administration to cells or to a subject.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating the support with a liquid suspension containing the cells. The impregnated supports obtained in this way can be implanted in a human subject. Such impregnated supports can also be re-cultured by immersing them in a liquid culture medium, prior to being finally implanted. The three-dimensional solid support needs to be biocompatible so as to enable it to be implanted in a human. It may be biodegradable or non-biodegradable.

The cells or cell populations can be administered in a manner that permits them to survive, grow, propagate and/or differentiate towards desired cell types (e.g. differentiation) or cell states. The cells or cell populations may be grafted to or may migrate to and engraft within the intended organ.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified intestinal epithelial cells, intestinal epithelial stem cells, or intestinal immune cells (preferably intestinal epithelial cells) and/or other active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

A further aspect of the invention provides a population of the epithelial cells, epithelial stem cells, or epithelial immune cells as taught herein. The terms “cell population” or “population” denote a set of cells having characteristics in common. The characteristics may include in particular the one or more marker(s) or gene or gene product signature(s) as taught herein. The epithelial cells, epithelial stem cells, or epithelial immune cells (preferably mucosal immune cells) cells as taught herein may be comprised in a cell population. By means of example, the specified cells may constitute at least 40% (by number) of all cells of the cell population, for example, at least 45%, preferably at least 50%, at least 55%, more preferably at least 60%, at least 65%, still more preferably at least 70%, at least 75%, even more preferably at least 80%, at least 85%, and yet more preferably at least 90%, at least 95%, at least 96%, at least 97%, at least 98%, at least 99%, or even 100% of all cells of the cell population.

The isolated intestinal epithelial cells, intestinal epithelial stem cells, or intestinal immune cells (preferably intestinal epithelial cells) of populations thereof as disclosed throughout this specification may be suitably cultured or cultivated in vitro. The term “in vitro” generally denotes outside, or external to, a body, e.g., an animal or human body. The term encompasses “ex vivo”.

The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

The term “medium” as used herein broadly encompasses any cell culture medium conducive to maintenance of cells, preferably conducive to proliferation of cells. Typically, the medium will be a liquid culture medium, which facilitates easy manipulation (e.g., decantation, pipetting, centrifugation, filtration, and such) thereof.

Adoptive Cell Transfer

Adoptive cell therapy can refer to the transfer of cells, most commonly immune-derived cells, back into the same patient or into a new recipient host with the goal of transferring the functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, adoptive cell transfer is used to modify one or more biomarkers to increase epithelial differentiation and/or restore homeostatic balance.

### EXAMPLES

### Example 1: Reduced Cellular Diversity and an Altered Basal Progenitor Cell State Informs Epithelial Barrier Dysfunction in Human Type 2 Immunity

Barrier tissue dysfunction is a poorly defined feature of chronic human inflammatory disease^{1,2}. The epithelium of the upper respiratory tract is the barrier responsible for separating inhaled agents, such as pathogens and allergens, from the underlying submucosa. Specialized epithelial subsets-including secretory, glandular, and ciliated cells-differentiate from basal progenitors to collectively realize this role³⁻⁵. Allergic inflammation in the upper airway barrier can develop from persistent activation of Type 2 immunity (T2I), resulting in the disease spectrum known as chronic rhinosinusitis (CRS), ranging from rhinitis to severe nasal polyps⁶⁻⁸. Basal cell hyperplasia is a hallmark of severe disease⁸⁻¹², yet how these progenitor cells^{3,13,14} relate to their healthy counterparts and functionally contribute to clinical presentation and barrier tissue dysfunction in humans remains unexplored. Profiling twelve primary human CRS samples surgically isolated from ethmoid sinus that span the range of clinical severity with the Seq-Well platform¹⁵ for massively-parallel single-cell RNA-sequencing (scRNA-seq), the first single-cell transcriptomes for human respiratory epithelial cell subsets, immune cells, and stromal cells (18,036 cells) from a T2I inflammatory disease are reported, and key mediators are mapped. Striking differences are found between the epithelial compartments of the non-polyp and polyp cellular ecosystems. More specifically, across 10,383 epithelial cells, a global reduction in epithelial diversity in polyps characterized by basal cell hyperplasia, a concomitant decrease in glandular and ciliated cells, and phenotypic shifts in secretory cell antimicrobial function is identified, which is validated through complementary methods in an orthogonal cohort. Through comparison with scrapings derived from healthy turbinate, inflamed turbinate, and polyp sinus tissues (additional 18,704 cells, 9 samples), core, healthy, inflamed, and polyp secretory cell signatures are defined. Furthermore, an aberrant basal progenitor differentiation trajectory in polyps is identified, and mechanistic cell-intrinsic and extrinsic factors that may lock polyp basal cells into this uncommitted state are proposed. Finally, it is functionally validated that basal cells ex vivo retain intrinsic memory of IL-4/IL-13 exposure, and the potential for clinical administration of in vivo IL-4Ra blockade to modify basal and secretory cell states is tested. Overall, the data define severe T2I barrier tissue dysfunction as a reduction in epithelial diversity, characterized by profound functional shifts stemming from basal cell defects, and identify a cellular mechanism for the persistence and chronicity of severe human respiratory disease.

T2I plays a dual role in regulating homeostatic processes, such as metabolism¹⁶, and promoting inflammatory defense mechanisms in response to parasites, venoms, allergens, and toxins¹⁷. Classically, T2I is characterized by sensor cells (epithelia, macrophages, dendritic cells, mast cells) producing first-order cytokines (TSLP, IL-25, IL-33) that cause release of second-order cytokines (IL-4, IL-5, IL-13, AREG) from lymphocytes⁵′1⁸. This, in turn, helps recruit effector cells (eosinophils, basophils, mast cells, monocytes), and induces epithelial remodeling, such as goblet cell hyperplasia⁹, to restore tissue integrity⁵⁸. Accumulating evidence suggests that immune modules, which are productively activated in acute settings to restore homeostasis, may reach an aberrant set point during chronic inflammatory diseases such as CRS^{20,21}. For example, cytokine modules may become self-reinforcing in T2I leading to substantial alterations in gross tissue architecture²² and the reduced epithelial barrier integrity characteristic of severe clinical presentations, such as polyps^{23,24}. How the overall human cellular tissue ecosystem shifts in composition and function during chronic T2I disease remains unknown. Specifically, within the epithelial compartment, while basal cell hyperplasia is a well-documented feature of severe allergic inflammatory diseases^{8-12,25-27}, it is unclear how human basal progenitor cells are intrinsically altered, and how this phenotype is mechanistically linked to barrier tissue dysfunction.

To directly address this in respiratory barrier tissue, Seq-Well¹⁵ (massively-parallel scRNA-seq for unique molecular identifier (UMI)-collapsed digital gene expression profiling) was used to profile surgically resected and dissociated ethmoid sinus tissue from patients across a spectrum of CRS severity (FIG. 1a; Methods; n=12 samples). Deconstructing these tissues into their component cells provides a unique lens into the cellular ecosystem of human T2I, allowing to: 1. characterize each major cell type without biases typically introduced by flow sorting/marker selection; 2. reconstruct tissue-level interactions; and, 3. rapidly assess which cell types/states have striking disease-associated transcriptional differences. To the latter point, the scRNA-seq data spans tissue specimens ranging from mild chronic inflammation and no eosinophilia, through moderate eosinophilia (the primary histologic signature of T2I), to more severe eosinophilic rhinosinusitis, presenting an opportunity to identify cellular features that correlate with T2I inflammatory disease severity and gross anatomical features, such as the presence of polyps (n_{Non-Polyp}=6; n_{polyp}=6;)⁸. As no healthy individual had a clinical indication for nasal tissue resections during enrollment (Methods), and nasal brushings cannot fully recapitulate the nasal mucosal ecosystem given a bias towards apical cells²⁸, globally assessing the cell types/states that inform the spectrum of T2I-mediated CRS was initially focused on (FIG. 1) and its gross anatomical manifestations (e.g., polyps vs non-polyps)^{5,8,23,24}.

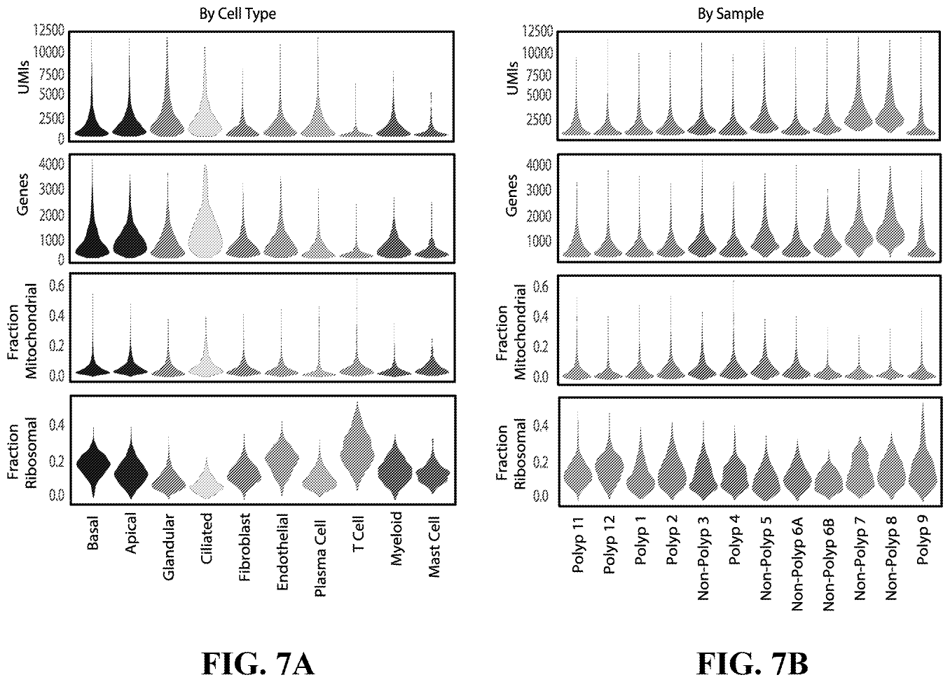

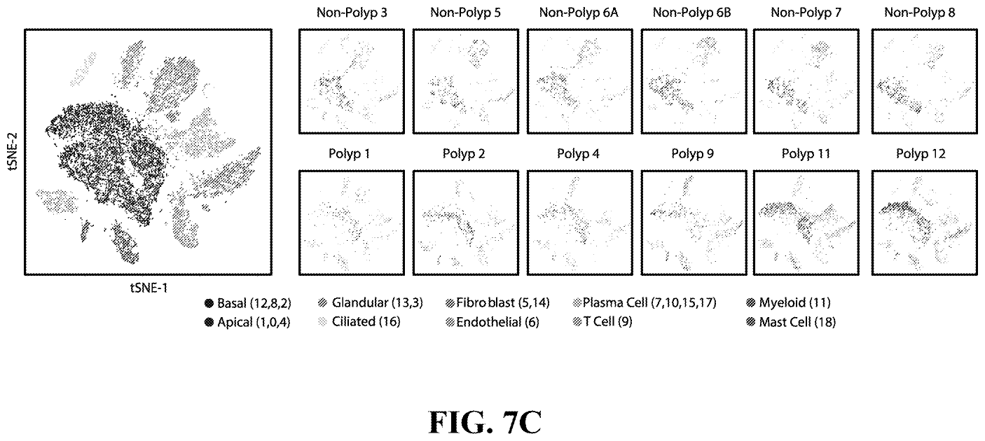

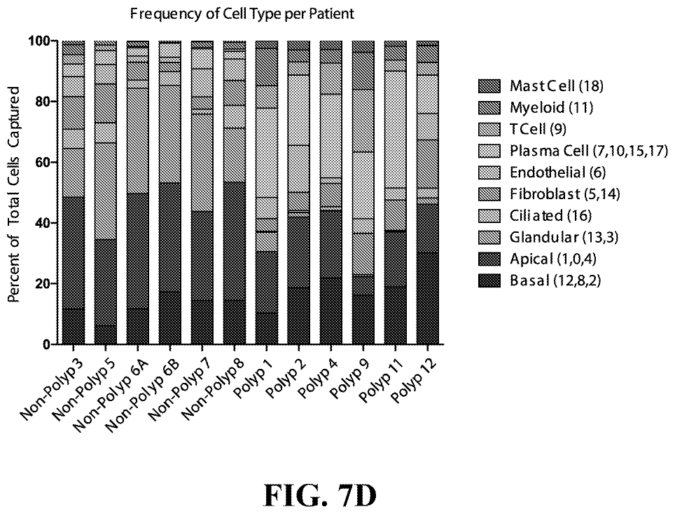

After library preparation and sequencing, a unified cells-by-genes expression matrix containing digital gene expression values for all cells passing quality thresholds was derived (n=18,036; FIG. 7a,b; Methods). Dimensionality reduction and graph-based clustering²⁹ were then performed (FIG. 1a; Methods). Identified clusters were present across all patients within the non-polyp and polyp groups, with no cluster composed solely of cells from a single individual (FIG. 7c,d). Lists of cluster-specific genes were subsequently derived (Table 1), and used to define the epithelial, immune, and stromal cell types²⁹ (FIG. 1b; FIG. 8a). From the complete lists of cluster-specific/sensitive markers (FIG. 8a, Table 1), several lineage-defining genes were displayed to highlight the major cell types recovered, including basal^{3,30} (KRT5) and apical^{3,30} (KRT8) to orient the major division present in pseudostratified epithelia of the respiratory tract, and further specialization present in ciliated^{3,31} (FOXJ1) and glandular^{32,33} (LTF) apical epithelium. The supportive endothelial³⁴ (DARC), fibroblast³⁵ (COL1A2), plasma³⁶ (CD79A), myeloid^{37,38} (HLA-DRA), T^{39,40} (TRBC2), and mast cells⁴¹ (TPSAB1) are also highlighted (FIG. 1c).

Despite the potential challenges associated with isolating single parenchymal and immune cells from tissues⁴², a highly reproducible distribution of cell types across patients was sequenced and annotated (FIG. 7c,d). Collectively, abundant cell numbers for all expected cell types across each patient were recovered, yet the exploratory nature of this study regarding specific disease endotypes is noted^{5,6,8,22} (FIG. 7d,). Given the rarity of ILC2s in unfractionated tissue⁴³, it would not be expected to detect them as a distinct cluster (Methods). The frequency of eosinophils expressing CLC (FIG. 8b) was lower than anticipated based on the literature⁸ and matched clinical pathology reports. Since a robust eosinophil and T-helper 2 (Th2) signature was measured in snap-frozen whole tissue nasal RNA-Seq libraries from nasal polyps⁸ (FIG. 8c,d,e), and they can be recovered by flow cytometry, the data suggest that eosinophil transcripts may be particularly susceptible to degradation during isolation, likely due to high levels of endogenous RNAses⁴⁴ (Methods).

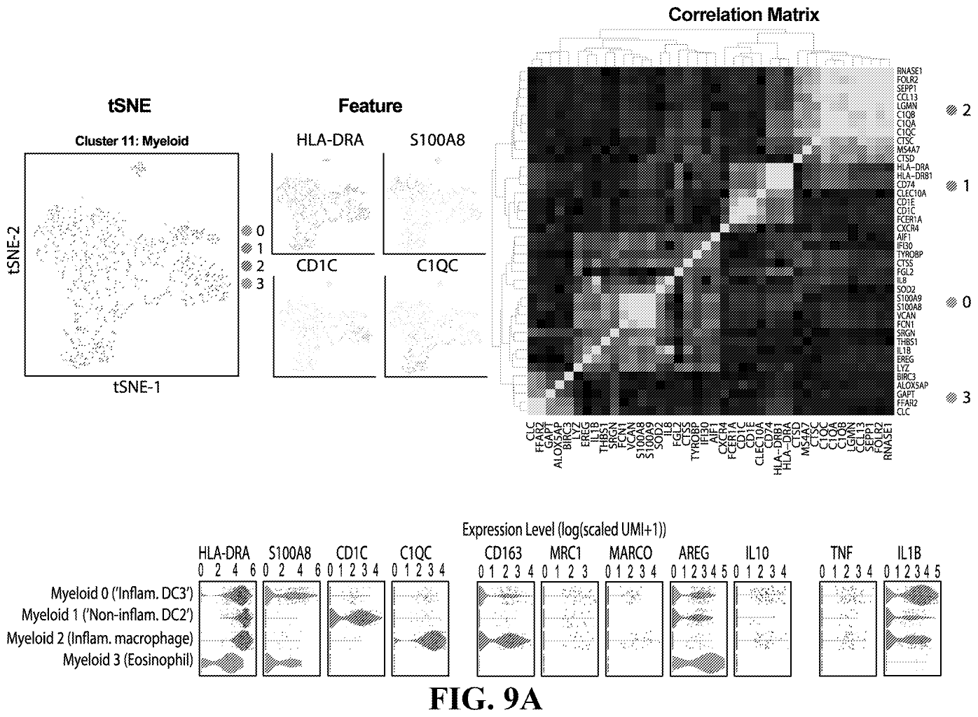

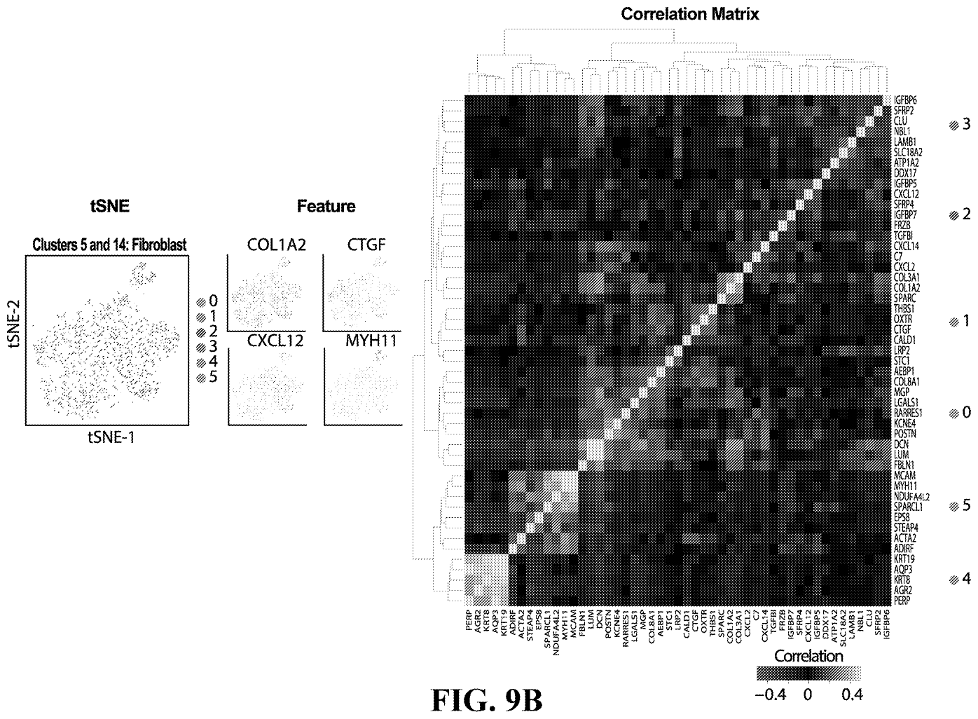

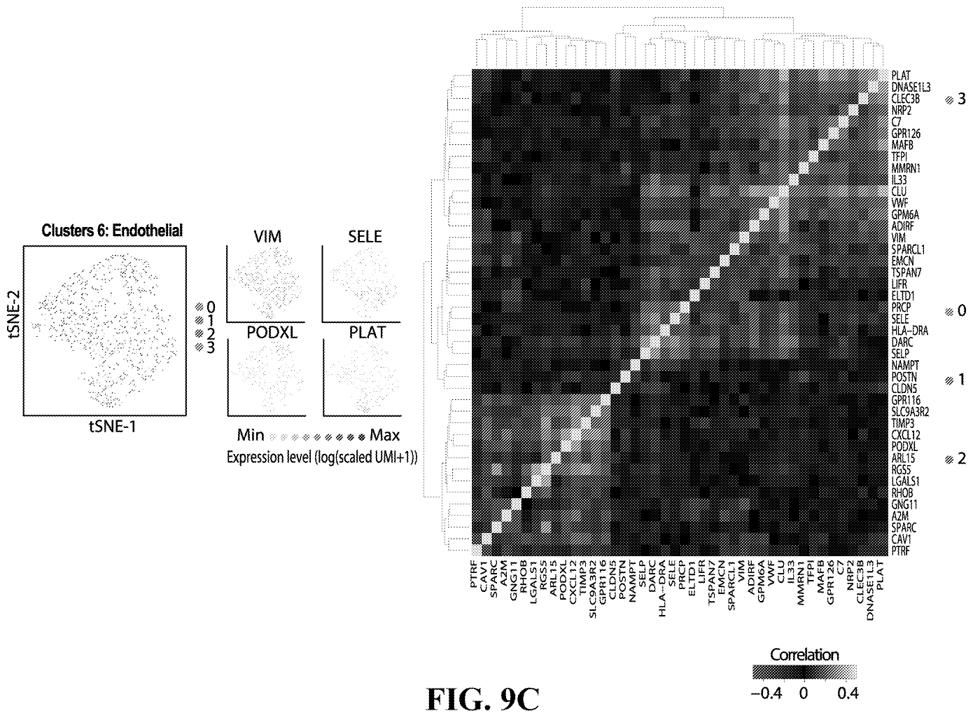

For each cell type recovered, sub-clustering reveled further, potentially meaningful heterogeneity (FIG. 9). For example, within the myeloid compartment, a small number of eosinophils (Myeloid-3) are identified, along with inflammatory macrophages (Myeloid-2), and two subsets of dendritic cells, expressing many of the marker genes of the recently described⁴⁵ ‘Non-inflammatory DC2’ (Myeloid-1) and ‘Inflammatory DC3’ (Myeloid-0) CD1c+ blood DCs, though the latter appear to lose expression of CDIC in tissue (FIG. 9a). Intriguingly, canonical genes associated with alternatively activated macrophages (CD163, MRC1, MARCO, IL10, AREG) are found distributed across macrophage and DC subsets co-expressing TNF and IL1B, highlighting the plasticity of these cells in tissues^{46,47} and the importance of studying these states in their native tissue setting (FIG. 9a). Similar analyses of fibroblasts reveal a spectrum of activation ranging from preferential production of chemokines to growth factors^{48,49} (FIG. 9b), and that endothelial cells are largely divided in two distinct classes, one of which represents post-capillary venules specialized for leukocyte recruitment (Endothelial-0)^{34,50} (FIG. 9c). While this study focuses largely on epithelial cells as well as selected interactions with immune and stromal cells in order to comprehensively characterize basal cell hyperplasia, it is expected that the data will serve as a useful reference to investigators with specific interests within each cell type, and will increase in value as other tissues are profiled across various inflammatory disease contexts. For example, it could be of interest to identify the tissue-specific nature of vascular beds specialized in the recruitment of immune cells to distinct organs^{34,50}

Lymphoid and myeloid cells are recruited and positioned in tissues to facilitate effector functions during T2I through the action of chemokines and lipid mediators, but the cell of origin for these molecules can be obscured by bulk cellular analyses^{6,51}. The eotaxins (CCL26, CCL11, CCL24) are chemotactic for eosinophils, acting through CCR3⁴⁴. In the scRNA-Seq data, each eotaxin was detected in distinct sources, including basal cells (CCL26), fibroblasts (CCL26 and CCL11), and myeloid cells (CCL24) (FIG. 1d). The mucosal cytokine CXCL17⁵² was largely detected in apical and glandular cells, while CCL28, a chemokine involved in the positioning of IgA+ plasma cells in mucosal tissues⁵³, was specifically detected in glandular epithelium (FIG. 1d). This cell-type distributed (eotaxins) or restricted (CCL28) expression pattern of chemokines may help to clarify recurrent patterns observed in histological studies of human tissue, explaining the diffuse distribution of eosinophils or the preferential recruitment of plasma cells to glandular epithelium, respectively²⁴.

Lipid mediators, such as prostaglandins and leukotrienes, also play key roles in T2I⁵⁴. Mast cells were found specifically enriched for HPGDS and PTGS2, and ALOX5, suggesting they may be a dominant source of prostaglandin D2, implicated in activation of Th2 cells via CRTH2 (FIG. 1d)⁵⁵, and leukotriene biosynthesis within CRS, respectively. Myeloid cells were enriched for TBXAS1, responsible for thromboxane A2 biosynthesis, and basal, apical and ciliated epithelial cells were high expressers of ALOX15, the key enzyme in synthesizing a mediator found in high levels in T2I, 15-HETE⁵⁶, and recently reported to regulate ferroptotic cell death in airway epithelial cells⁵⁷ (FIG. 1d).

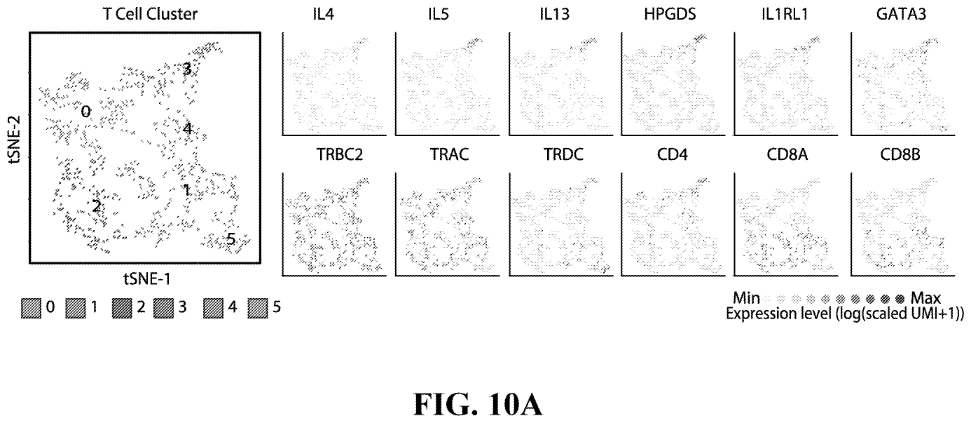

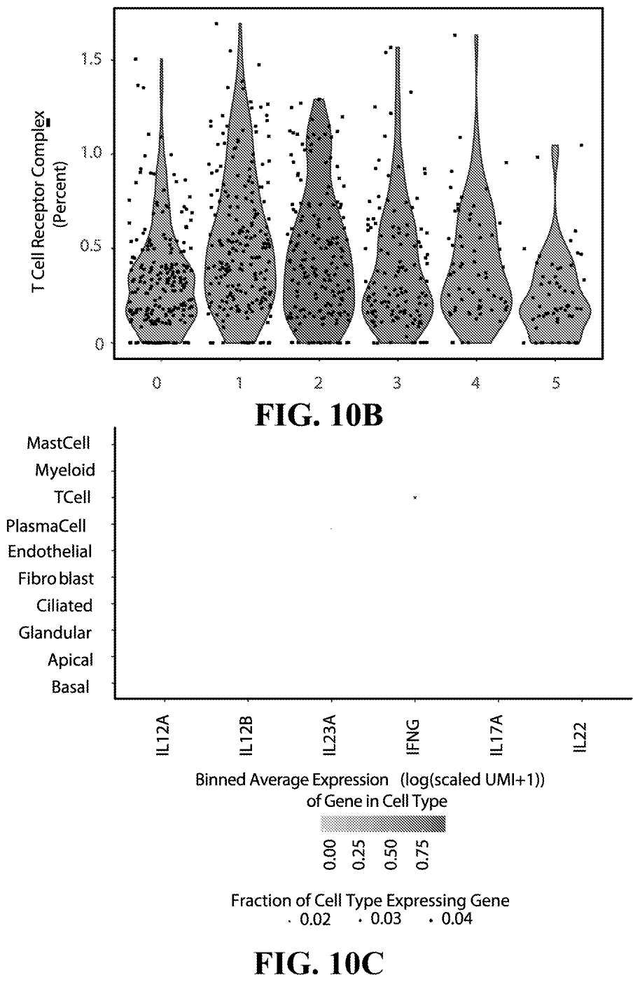

The production of instructive first-order cytokines, alongside these chemokines and lipid mediators, primes recruitment and activation of effector mechanisms. In particular, IL-25⁵⁸, IL-33⁵⁹⁻⁶¹, and TSLP^{62,63} are broadly referred to as epithelial-derived cytokines^{5,6,51}, and yet little is known about their cell-of-origin in human disease. Consistent with previous reports, IL25 was not detected in the system⁵⁸ (FIG. 1e). IL33 was present in basal cells, identifying their contribution to both upper and lower airway disease⁶⁴, but was also localized to apical and ciliated cells, highlighting important differences across disease states and tissues. However, the key instructive cytokine TSLP was uniquely restricted to basal cells, which may link increases in basal cell number to observed activation of effector cells associated with allergic inflammation (FIG. 10a), potentially through its polarizing actions on DCs^{65,66} (FIG. 1e, FIG. 9a). Furthermore, amplifying cytokines (e.g. IL-1 family members)⁵ were either distributed throughout both epithelial and immune subsets (IL18), or limited to myeloid cells (IL1B) (FIG. 1e; FIG. 9a).

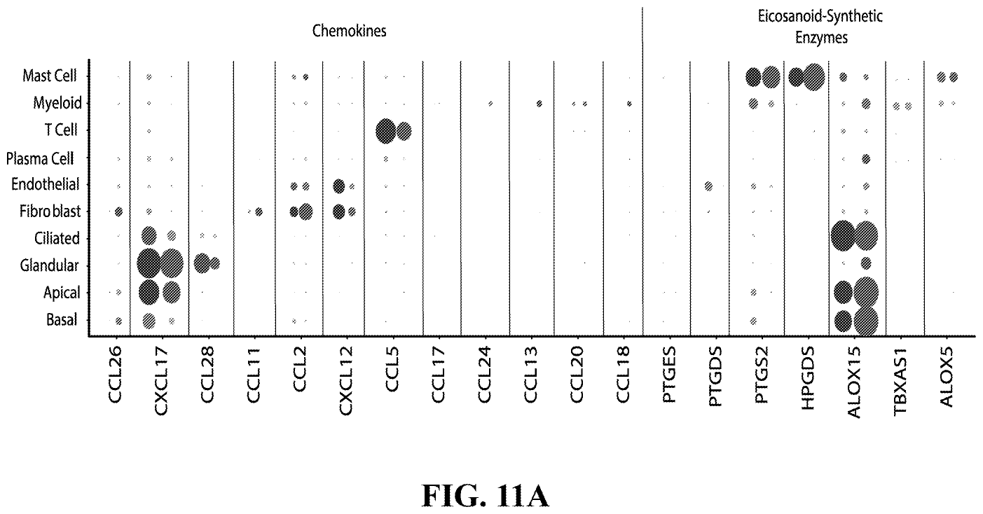

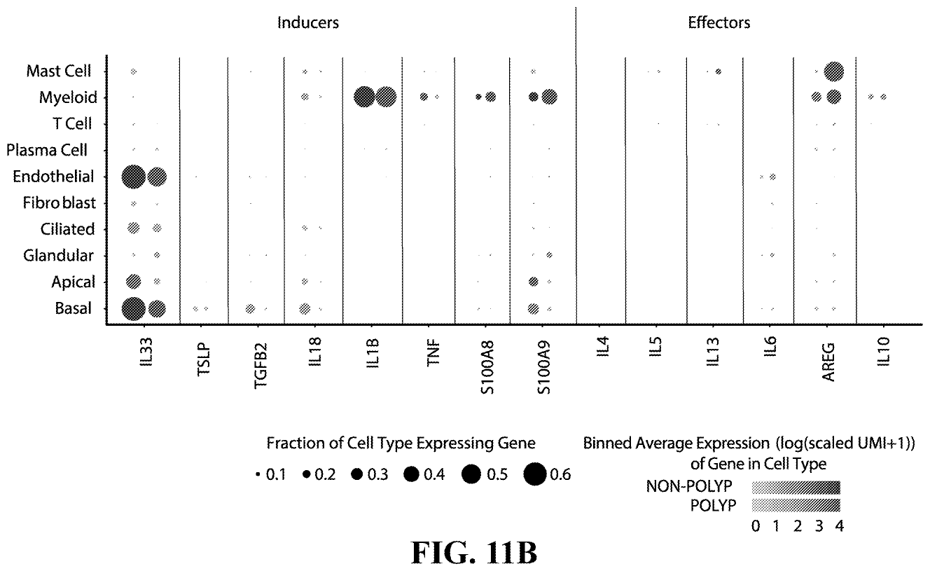

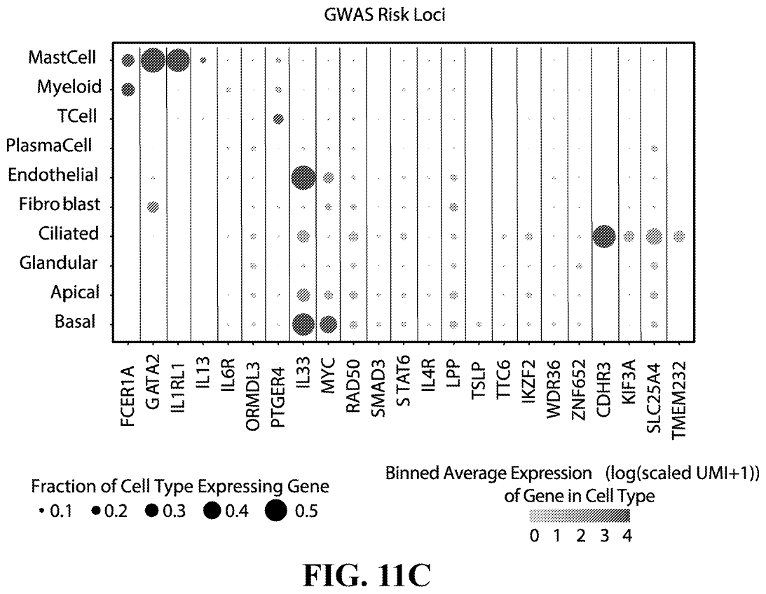

With regard to second-order cytokines produced in response to TSLP and IL-33, the small, but consistent, subset of CD4+ T cells expressing IL4, IL5, IL13, also expressed HPGDS, fitting the recently described profile of allergen-specific Th2A cells⁶⁷ (FIG. 10a; Methods). All identified T cell sub-clusters scored evenly for a set of TCR complex genes (FIG. 10b; Methods), and robust signals could not be detected for Type-1 or Type-17 inducer or effector cytokines across any cell subset (FIG. 10c). Beyond T cells, a substantial number of mast cells expressing IL5 and IL13 were also noted (FIG. 1e)⁶². Furthermore, mast cells along with myeloid cells were the main expressers of the tissue-reparative cytokine AREG⁶⁸ (FIG. 1e), which has largely been attributed to regulatory T cells and ILC2s in murine in vivo studies⁶⁹. Notably, patients with or without polyps showed consistent cells-of-origin for the T2I-related chemokines, lipids, and cytokines, except for select mediators, such as CCL26 and AREG, which were more broadly detected in polyp tissue, and PTGS2 (e.g. COX2), which was reduced in polyp epithelium (FIG. 11a,b). Intriguingly the reduction in COX-2 from epithelial cells in polyps would help explain the high PGD2/PGE2 ratios associated with increasing disease severity, as mast cells primarily synthesize PGD2 while epithelial cells are capable of PGE2 synthesis⁷⁰.

Of specific interest in the context of human diseased tissues is leveraging the literature on genome-wide association studies (GWAS) for allergic diseases⁷¹. While several risk genes have been identified, their links with phenotype have proven complex to unravel⁷². In many cases, the precise pattern of expression within a complex tissue has not formally been clarified due to technological limitations of bulk gene expression analyses. It was noted that several risk genes (IL13, IL33, TSLP) mapped to a restricted set of cell types (FIG. 1d,e). Thus, the distribution of risk alleles throughout the cell types were formally investigated, identifying GATA2 and ILIRL1 (IL-33R) enriched in mast cells, and CDHR3, KIF3A and 7MEM232 specifically mapping to ciliated cells (FIG. 11c). While current GWAS literature describes the rhinovirus C receptor CDHR3 as an epithelial-enriched gene, the fine-mapping of CDHR3 to ciliated cells⁷³ could provide novel therapeutic angles such as supporting cilium motility in at-risk individuals⁷⁴. Furthermore, enrichment of ubiquitously expressed genes such as MYC to basal cells indicates targeted investigation of MYC-related programs specifically within this cell type during chronic allergic inflammation⁷⁵ (FIG. 11c). Cellular maps of tissues frequently affected by inflammatory disease will aid in providing mechanistic insight to genotype-phenotype interactions through the identification of specific cell types affected and broader risk-gene-associated genetic modules. Overall, the data highlight the activation of similar T2I modules across CRS tissues, implicate basal cells as key producers of first-order cytokines with important implications for diseases where basal cell hyperplasia arises^{8-12,25-27}, and CD4+ T cells and mast cells as key producers of second-order cytokines with the potential to influence epithelial cell states^{76,77}.

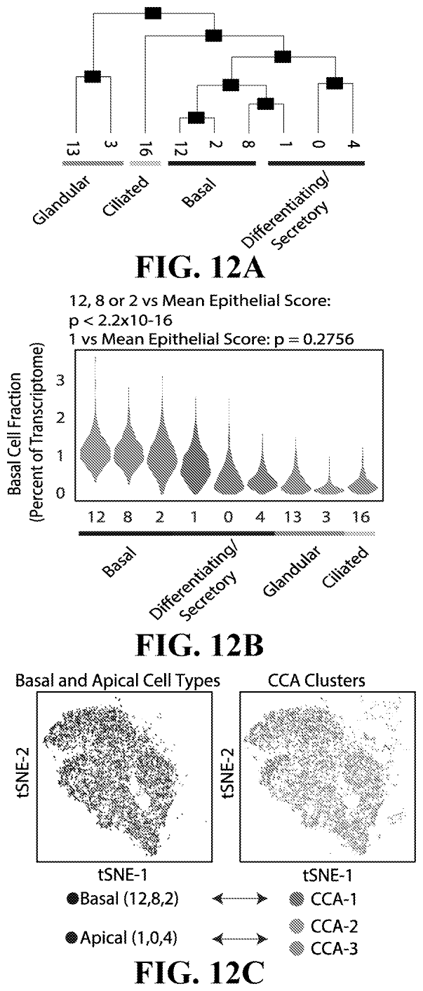

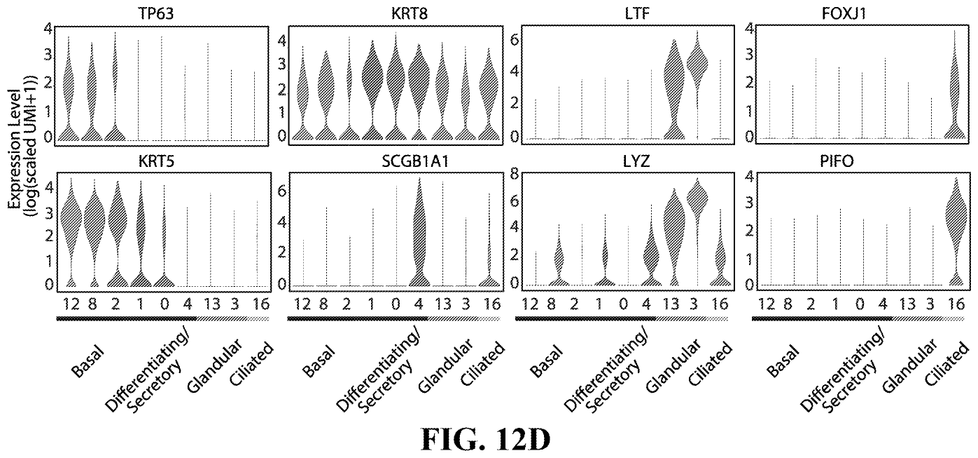

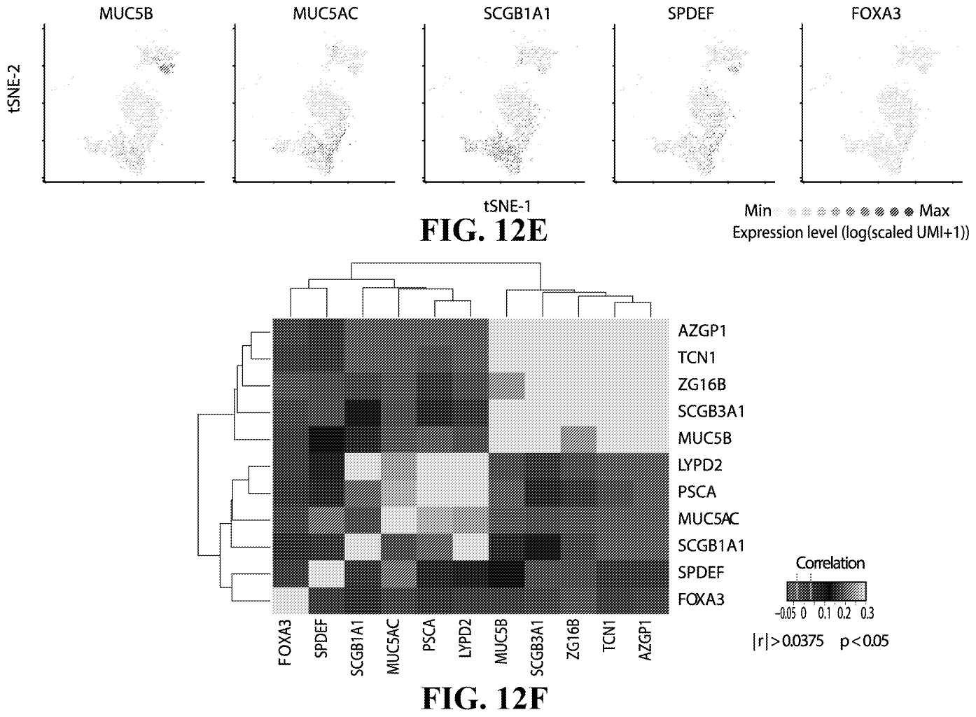

To better understand the epithelial cell states present across CRS, clusters within the broad epithelial cell populations were further analyzed (FIG. 2a; FIG. 12a,b,c). The first single-cell human transcriptomes in the absence of flow-sorting for basal⁷⁸, secretory, glandular, and ciliated cell types from a T2I ecosystem were provided (FIG. 2a,b,c; FIG. 12) Marker gene analysis across epithelial cell types (e.g. basal vs. differentiating/secretory vs. glandular vs. ciliated) identified conserved programs present in the three clusters with a basal phenotype (TP63, KRT5, and high basal cell score¹⁴), the three with a differentiating/secretory phenotype (KRT8, SERPINB3, SCGB1A1, and baseline basal cell score¹⁴), the two with a glandular phenotype (LTF, TCN1, LYZ), and the one with a ciliated phenotype (CAPS, OMG, FOXJ1, PIFO; FIG. 2a,b; FIG. 12a,b,d)^{3,4}Marker gene analysis within each epithelial cell type (e.g. 12 vs. 8 vs. 2 only) revealed further granularity associated with the presence or absence of polyps (FIG. 2c,d;). Within basal cells and differentiating/secretory cells, canonical Type 2 cytokine induced genes (POSTN⁷⁹ and ALOX15) were observed amongst others, drive clustering, suggesting that the separations observed may be mediated by differences in the sensing or impact of cytokines between disease states (FIG. 2c,d). Importantly, a canonical correlation analysis (CCA)⁸⁰, performed across non-polyp and polyp samples, returned similar basal and differentiating/secretory cluster groupings (FIG. 12c).

Next, the divergence in tissue remodeling present in the cohort was leveraged to understand the functional differences associated with less (non-polyp) and more (polyp) severe T2I inflammatory disease (FIG. 2d). Striking differences were observed within specific cell clusters driven by genes strongly associated with polyposis (FIG. 2b,c), and the numerical over-representation of cells from non-polyp and polyp ecosystems within each cluster and type were quantified. The clusters composing basal, differentiating/secretory, and glandular cell types showed the most significant links to disease-state (p-values by Fisher's with least significant difference; FIG. 2d). Nasal polyps are hypothesized to arise from the presence of opportunistic or pathogenic microorganisms⁸. Thus, the transcriptomes of differentiating/secretory cells (containing KRT8 secretory and goblet cells) (FIG. 2e;), which typically serve important antimicrobial functions through secreted products⁴, were first compared. In non-polyp epithelium, robust expression of antimicrobials (MSMB, SCGB1A1, STEAP4, PSCA, and LYPD2) was observed, which were diminished in polyp epithelium (FIG. 2e). In contrast, secretory cells recovered from polyps expressed CST4 and CST1, associated with inhibition of protease activity⁸¹, and IGFBP3, TFF3, and EGLN3, indicating attempts at curtailing tissue growth⁸² and promoting tissue tolerance and repair^{5,83,84} (FIG. 2e). Thus, secretory cells from polyps appear to supplant antimicrobial functions with tissue-reparative ones.

The two predominant secreted mucins of the upper respiratory tract are MUC5B and MUC5AC, which differ in their biophysical properties and potential to mediate microbe adhesion⁸⁵. Although the production of both has been commonly ascribed to a set of goblet cells in human airway⁴, expression of MUC5B is observed to be restricted to glandular cells (cluster 13) in nasal mucosa, which do not express MUC5AC (r=−0.0375; p=0.4952, corrected Holm's method; FIG. 2c; FIG. 12e). Instead, MUC5AC is expressed in a subset of secretory cells (clusters 0 and 4) co-expressing SCGB1A1 and FOXA3 (MUC5B vs AZGP1: r=0.491, MUC5AC vs AZGP1: r=−0.084, MUC5AC vs SCGB1A1: r=0.184, MUC5AC vs FOXA3: r=0.181; p<0.0001, corrected Holm's method; FIG. 12e,f). This suggests that the goblet cell program is layered atop a secretory cell base⁸⁶⁻⁹⁰, and that glandular cells are the predominant source of MUC5B⁹¹. Expression of SPDEF, a putative goblet cell transcription factor⁴, was shared amongst mucin producing cells, but relatively enriched in MUC5AC expressing cells (SPDEF vs MUC5B: r=0.107, SPDEF vs MUC5AC: r=0.213; p<0.0001, corrected Holm's method) (FIG. 12e,f). Crucially, MUC5AC and MUC5B are not functionally interchangeable-in murine models, MUC5B, but not MUC5AC, is essential for maintaining immune homeostasis and controlling infections of the upper airway, primarily through promoting mucocilliary clearance⁹²—suggesting imbalances among these cell types could have profound effects on host defense.

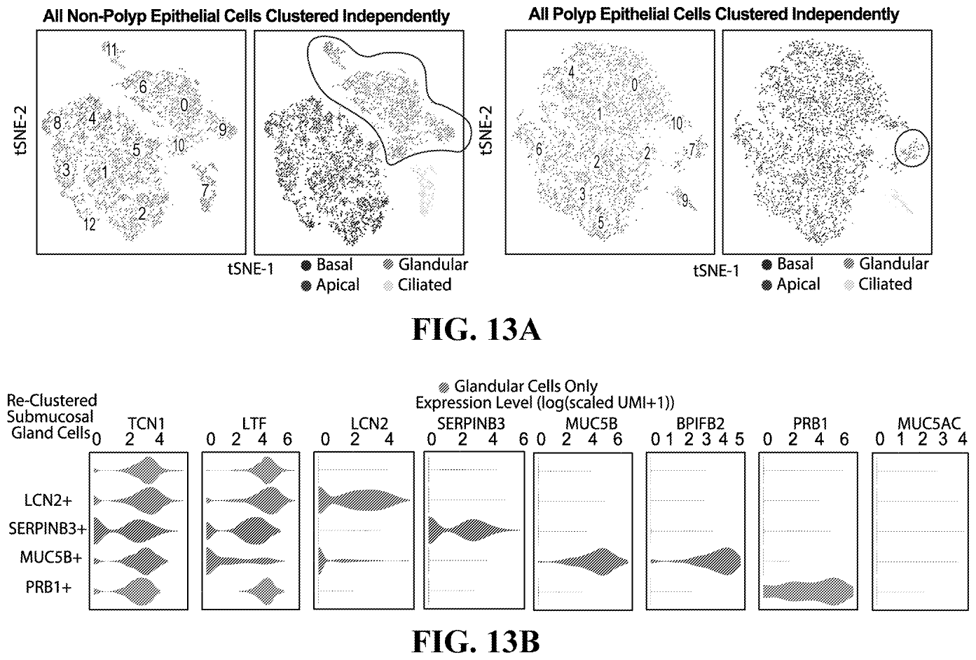

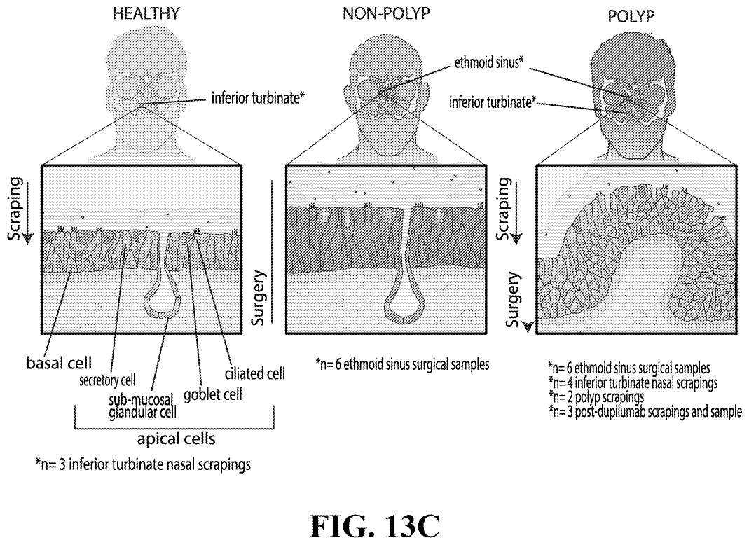

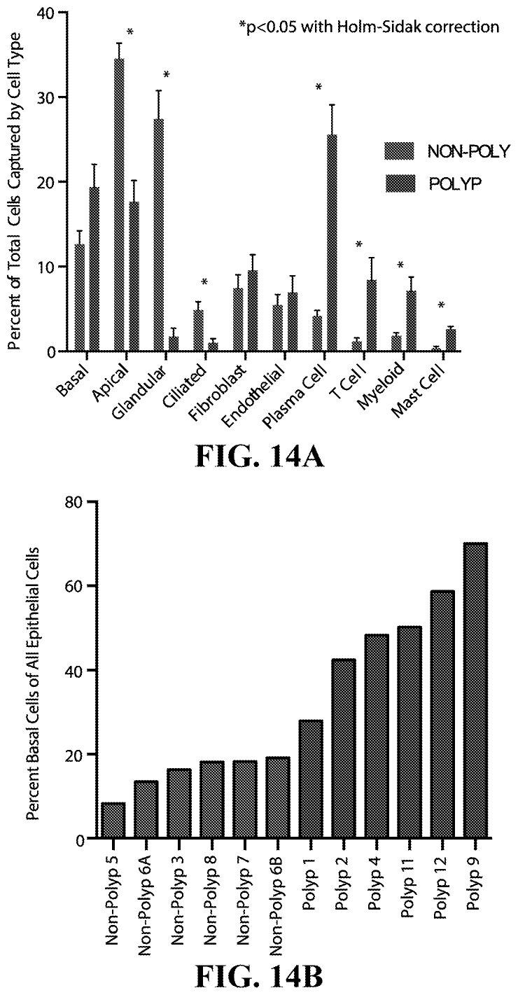

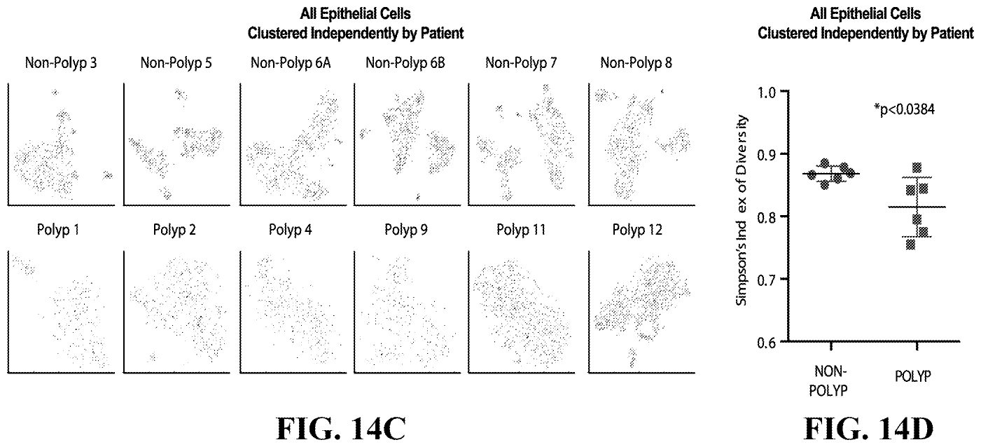

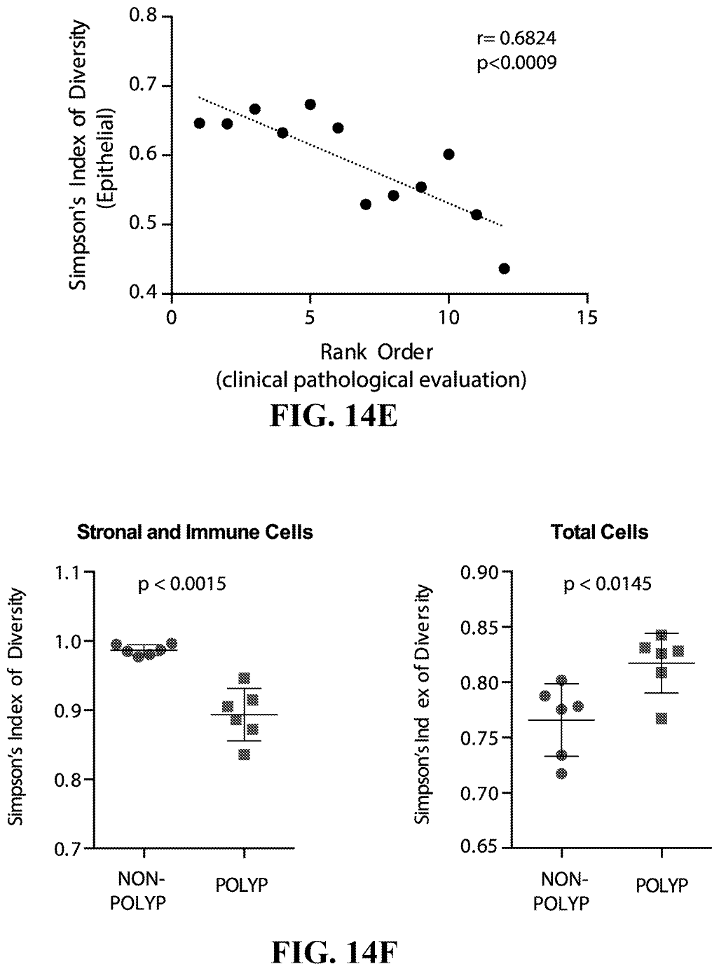

Having the restricted expression of MUC5B to a subset of glandular cells been identified, the heterogeneity present within all glandular cells⁹³ was more formally assessed (clusters 3 and 13, FIG. 2a). All glandular cells within the epithelial cells were isolated (FIG. 13a) and re-clustered independently finding five main clusters (FIG. 13b). Importantly, each cluster had cells from all non-polyp samples, suggesting that these are not patient-specific subsets. All clusters shared expression of TCN1, and the majority expressed LTF, but expression of LCN2, SERPINB3, MUC5B and BPIFB2, and PRB1 were restricted to specific subsets^{94,95} (FIG. 13b). This expression from restricted subsets may represent a regulatory mechanism for controlling the production and release of certain antimicrobial factors and secretory proteins without inducing global release of gene products^{96,97}. No comparison between polyp and non-polyp glandular and ciliated cells was possible as these clusters were nearly absent from polyp ecosystems (FIG. 2d; FIG. 13; FIG. 14a).

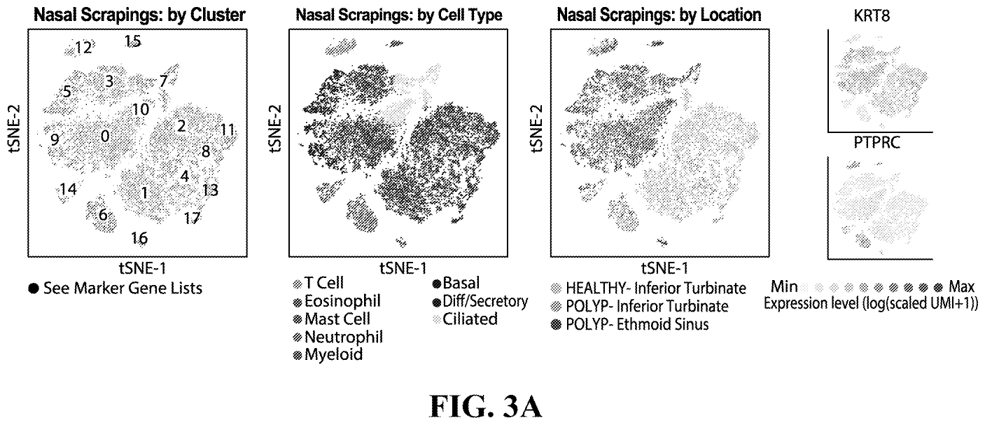

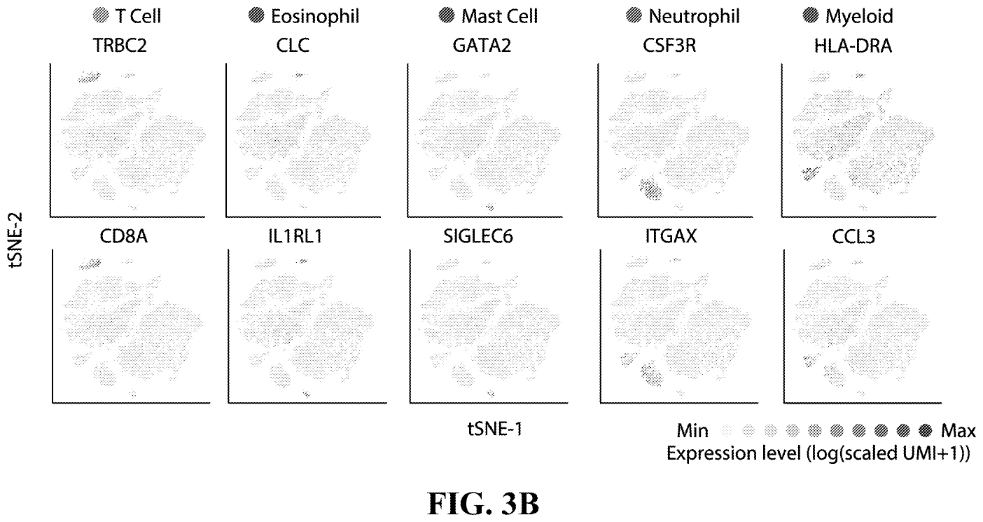

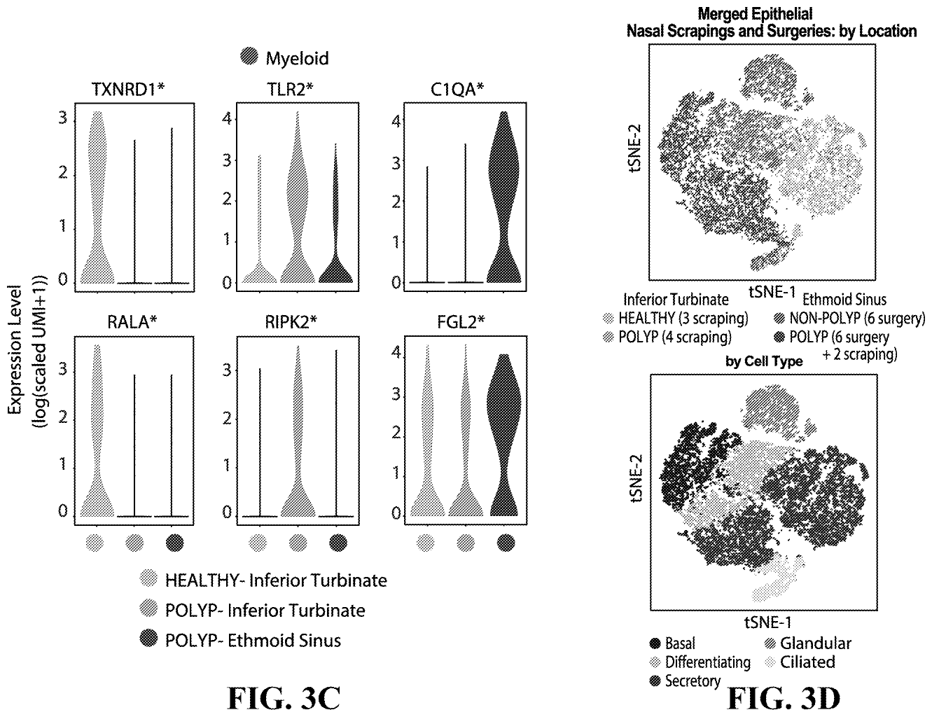

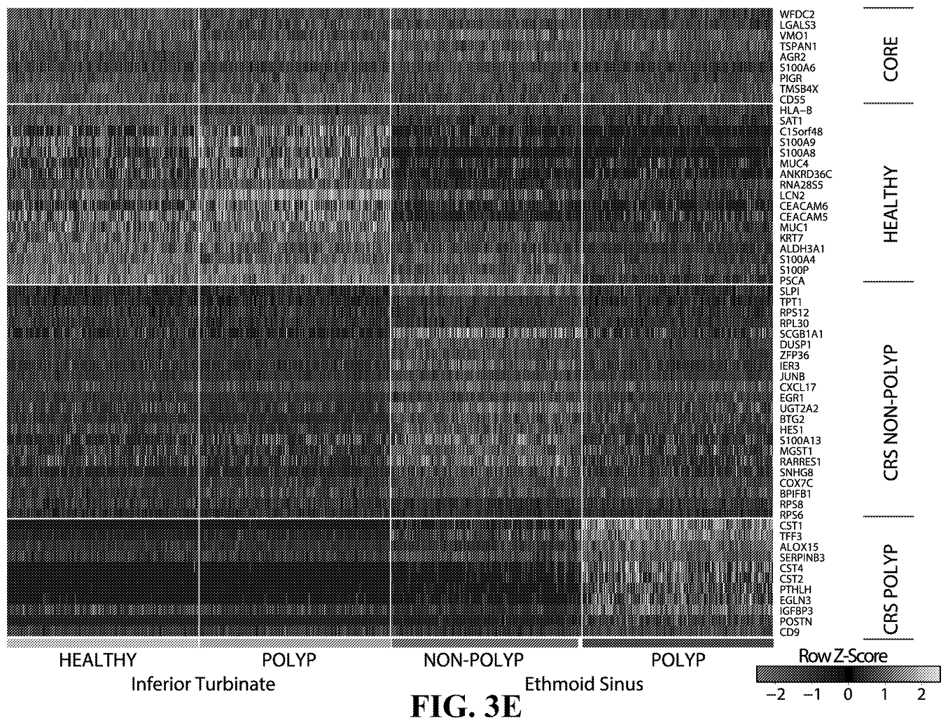

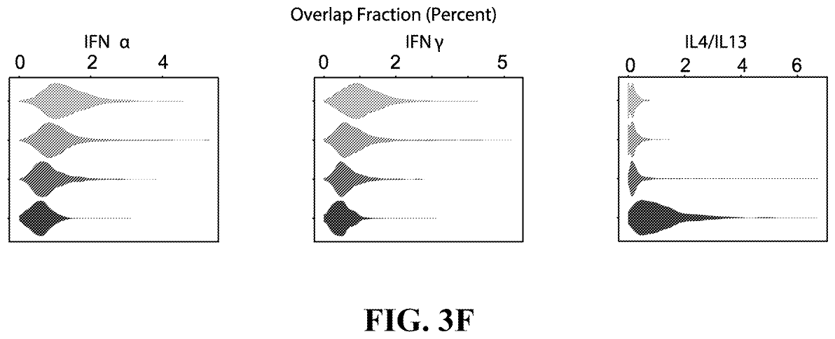

To help contextualize disease-induced shifts in apical cell types, and to confirm correct identification of sub-mucosal glandular cells in our data, sino-nasal scrapings were used as a method of sampling healthy tissue⁹⁸. Nasal scraping allows for access to the superficial epithelial cell layer of the inferior turbinate; in contrast, the surgical resections from ethmoid sinus utilized as the central data set of this study contain both epithelial cells and underlying tissue, including sub-mucosal glands (FIG. 13c). Since this method samples a proximal but distinct anatomical location with a distinct technique, in addition to collecting inferior turbinate scrapings from healthy controls (n=3), inferior turbinate scrapings from individuals with polyps (n=4), and, from two of these individuals, from accessible polyps protruding beyond the middle meatus (n=2) were also collected. Massively-parallel scRNA-seq using Seq-Well was performed (FIG. 3a, Methods), and 18,704 additional single cells were recovered, which were analyzed using the principles applied above (FIG. 1a,b;) to derive marker gene lists and identify cell types (FIG. 3a). Differentiating/secretory and ciliated epithelial cells were recovered from the inferior turbinate of both patients with polyposis and healthy controls, but only identified basal cells in polyp scrapings (FIG. 3a). Comparisons of nasal scraping tSNE plots colored by location/diagnosis and by cell type reveal a striking separation between the epithelial cells from the inferior turbinates and from polyps, yet a remarkable similarity in the inferior turbinate of healthy controls and individuals with polyps (FIG. 3a), underscoring the importance of local cues in driving disease at barrier sites. T cells, neutrophils, and myeloid cells were recovered from both anatomical locations, while mast cells and eosinophils were exclusively found in polyp epithelium (FIG. 3a,b). Genes of interest enriched within each immune cell type were displayed (FIG. 3b), and, as myeloid cells were sampled evenly across all three sites, the opportunity was taken to identify genes preferentially expressed within each region sampled (FIG. 3c). From healthy inferior turbinate, cells expressed TXNRD1 and RALA involved in an anti-inflammatory macrophage phenotype⁹⁹ and phagocytic function¹⁰⁰, from polyp inferior turbinate cells TLR2 and RIPK2, involved in microbial sensing¹⁰¹, and from polyp tissue cells C1QA and FGL2 indicative of the pro-inflammatory environment¹⁰² (FIG. 3c).

To identify conserved gene modules in secretory cells, as well as shifts in cell states across anatomical regions in health and disease, all epithelial cells recovered from surgical resections of non-polyp and polyp ethmoid sinus tissue were combined (FIG. 2) with scrapings from healthy inferior turbinate, polyp inferior turbinate, and accessible polyp tissue (FIG. 3d). Importantly, glandular cells continued to cluster separately, and no cells scoring for the glandular marker gene set were recovered (FIG. 2b;) through scraping. This suggests that glandular epithelium in the original data set and in iterative clustering was correctly classified (FIG. 13). Using marker discovery for secretory cells across all samples led to the identification of a conserved core gene set found across secretory cells from all diseases and sites sampled¹⁰³⁻¹⁰⁵, including WFDC2, VMO1, TSPAN1, AGR2, S100A6, PIGR, and CD55 (Core: FIG. 3d,e). Extracting all secretory cells across the four categories and performing marker discovery, a set of genes (S100A8/A9, MUC4, ANKRD36C, LCN2, and others)^{94,106,107} were found shared across healthy and polyp inferior turbinate (Healthy: FIG. 3e), with only slightly elevated expression of some markers in inferior turbinate from patients with polyps. By relating non-polyp and polyp ethmoid sinus secretory cells to healthy inferior turbinate secretory cells, a significant decrease in S100A8/A9, MUC4, ANKRD36C, and MUC1 is found, highlighting a conserved alteration in secretory cell state in CRS tissue (CRS: FIG. 3e; inferior turbinate vs. ethmoid sinus p<5.43×10⁻¹⁸⁶ or less with Bonferroni correction for multiple comparisons). Nevertheless, in non-polyp secretory cells relative to all others, an increase in SCGB1A1, UGT2A2, SLPI, S100A13, and RARRES1 is found, suggesting that non-polyp secretory cells are altered in the typical production seen in healthy and polyp inferior turbinate tissues to yield a distinct cell state¹⁰⁸⁻¹¹⁰ (CRS non-polyp: FIG. 3e). Additionally, there is an overall loss of antimicrobial effector programs in polyp secretory cells, whether compared to non-polyp secretory cells from the ethmoid sinus (FIG. 2e, FIG. 3e), or either condition in the inferior turbinate (CRS polyp: FIG. 3e). Using gene lists derived from cytokine-treated air-liquid interface (ALI) cultures (Methods), IFNα- and IFNγ-induced genes¹¹¹ are found enriched in healthy inferior turbinate, and there is a progressive loss of these signatures through CRS tissue accompanied by a large increase in IL-4/IL-13 induced genes¹¹¹ such as CST1, ALOX15, and SERPINB3, in polyp secretory cells (CRS polyp: FIG. 3e,f). It is concluded that secretory cells from involved CRS tissue of the ethmoid sinus differ significantly from the inferior turbinate, and that secretory cells in non-polyp and polyp reach distinct states whose altered functionality may be linked to disease trajectory and severity.

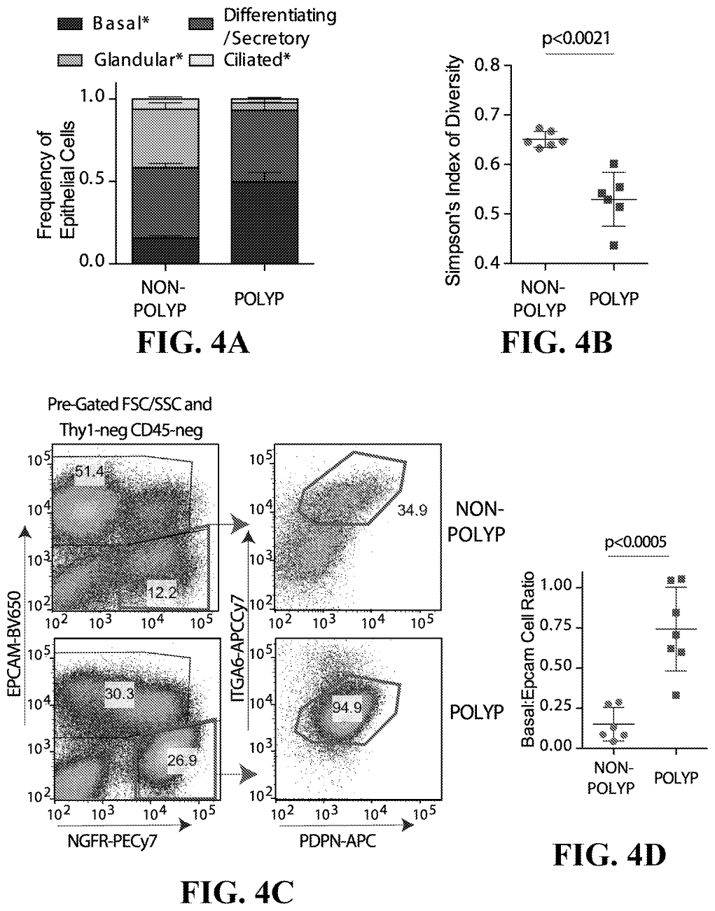

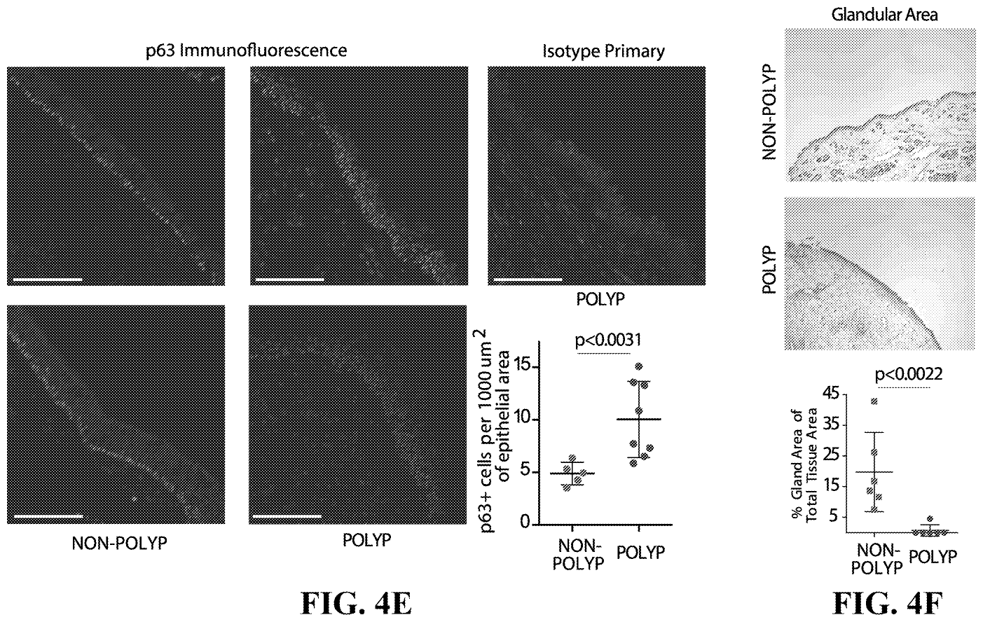

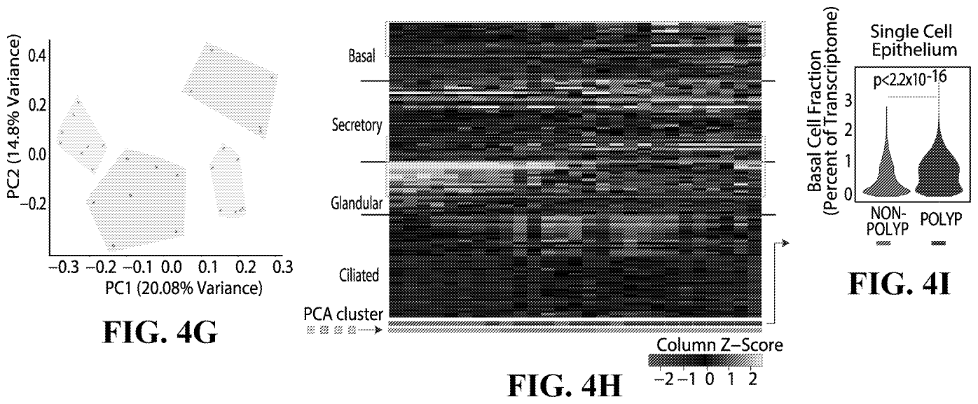

As basal progenitor cells give rise to the specialized cell types present in the epithelium^{3,14}, and glandular and ciliated cells were significantly reduced among polyps (FIG. 4a, FIG. 14a), the distribution of epithelial cell types/states present in each sample was more formally examined. Appropriate biodiversity is key to the robustness of any ecological system¹¹². Thus, Simpson's index of diversity¹³—a measure of the richness of an ecosystem—is used to characterize the composition of epithelial cells across basal, differentiating/secretory, glandular, and ciliated groupings in the non-polyp and polyp ethmoid sinus tissue ecosystems, as this metric accounts for both the number of distinct cell types present (e.g. species), and the evenness of the cellular composition across those cell types (e.g. relative abundance of species to each other; Methods). These data indicate a significant loss of epithelial diversity in nasal polyps, largely stemming from the aforementioned decrease in glandular and ciliated cells, and an enrichment in basal cells (FIG. 4b) that tracks the spectrum observed via clinical diagnoses (FIG. 14b). To test if these findings were driven by biases introduced in cell type curation, the number of clusters present within epithelial cells from each patient is calculated independently and Simpson's index of diversity for each is also calculated, obtaining consistent results (FIG. 14c,d). Furthermore, the rank-ordered pathological assessment of the patient tissue samples positively correlated with basal cell frequency (r=0.6252) and negatively with epithelial diversity (r=−0.6824; FIG. 14e). Intriguingly, when calculating Simpson's index of diversity across stromal and immune cells alone (e.g. fibroblast, endothelium, immune), or the entire cellular ecosystem for each patient, a reduced index of diversity in polyps for stromal and immune cells, but an overall increase across all cell types was observed (FIG. 14f). Given the relative distribution of cell types present (FIG. 14a), and how evenness factors into this equation, it is speculated that the immune cells in polyps may represent an overcorrection in attempting to restore balance to the epithelial compartment.

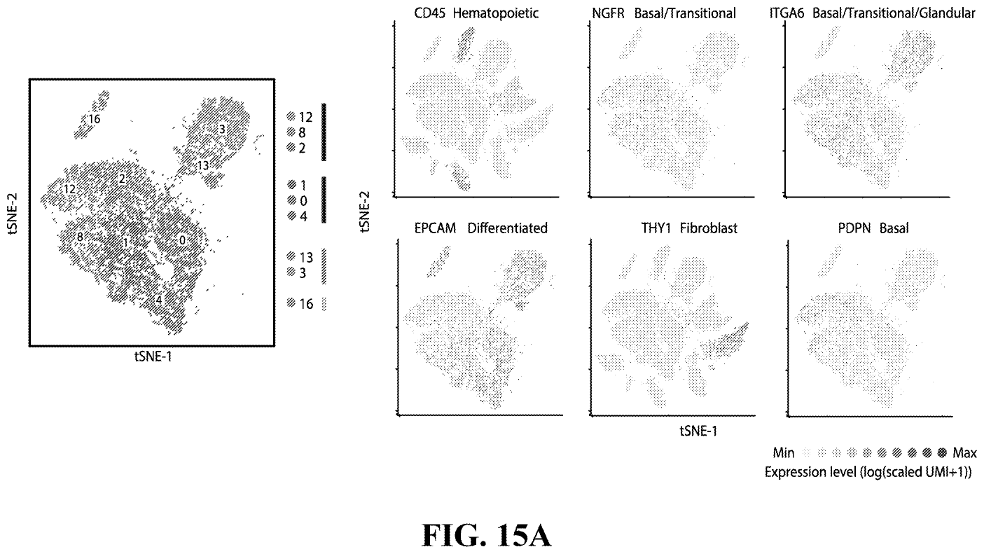

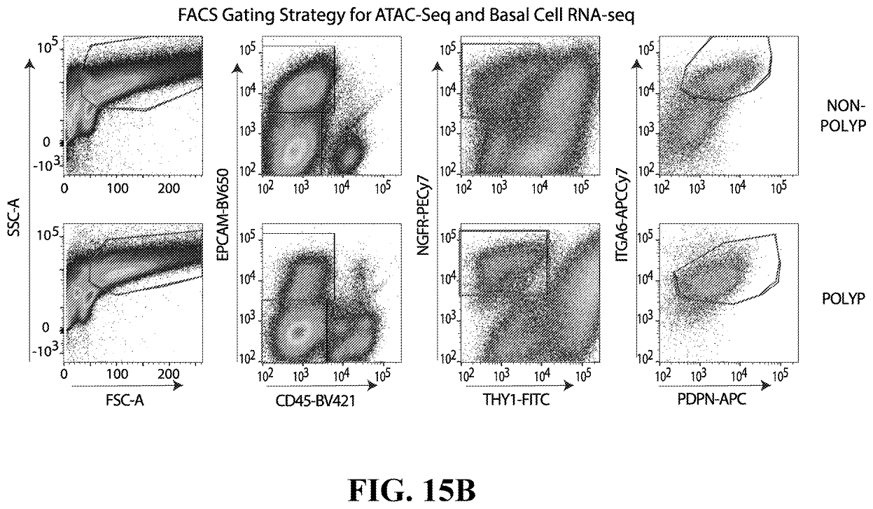

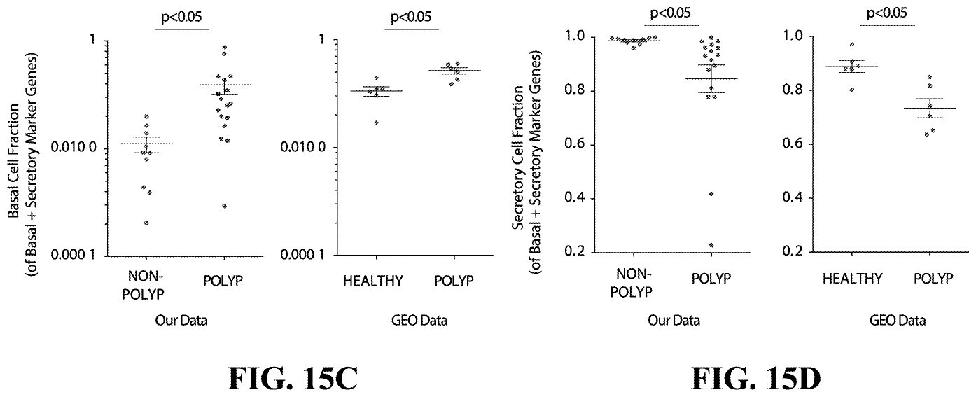

To confirm the epithelial findings, several complementary approaches were utilized. First, leveraging a flow cytometric gating strategy for human basal cells from the published literature¹⁴ (FIG. 4c; FIG. 15a,b), the frequency of basal cells is found significantly increased in ethmoid sinus polyps at the expense of differentiated epithelial cells in an orthogonal cohort of 13 additional patients (FIG. 4d). Second, using histology to ensure that the scRNA-Seq and flow results could not be explained as a dissociation induced artifact, a significant increase in the number of p63+ cells per 1000 μm² of barrier tissue area in the cohort, and a striking loss of glands in ethmoid sinus polyps, is confirmed^{10,11} in direct accordance with the scRNA-seq data (FIG. 4e,f; Methods). Finally, utilizing marker genes for specialized lineages ( ), bulk ethmoid sinus tissue RNA-seq of another orthogonal cohort (n=27 patients) was deconvolved to look more globally at intact tissues (which, without curation, were largely driven by immune signatures; FIG. 8c,d,e). Focusing on genes that define human basal, secretory, glandular, and ciliated cell subsets, four clusters of patients were identified (K-nearest neighbors (KNN) on a principal components analysis (PCA); FIG. 4g,h; Methods): a non-polyp cluster (grey) enriched in secretory and glandular signatures and then three increasingly polyp-enriched clusters that showed more pronounced basal and ciliated cell programs (cyan), then lost glandular ones (lilac), and eventually, in the most severe cases, lost core ciliated genes (coral, as determined by number of previous surgeries, time to polyp regrowth, and eosinophilia; FIG. 4h; Supplementary Table 1,). Within polyps, upregulation of the tissue reparative program observed in FIG. 2e for differentiating/secretory cells was also confirmed (FIG. 4h). Serial profiling starting at incipient disease will be required to investigate whether CRS is divisible into a strict clinical divergence, or if individuals with polyps traverse a disease landscape from healthy tissue through that observed in non-polyps en route to polyposis.

To address if basal cell hyperplasia, in addition to loss of secretory cell function (FIGS. 3 and 4), characterizes deviations from healthy tissue, as well as from the CRS non-polyp disease state, two publicly-available RNA-seq datasets containing normal human sinus mucosal biopsies, and non-eosinophilic and eosinophilic nasal polyps^{8,114,115} were used. Re-analyzing each sample for the fraction of basal or secretory cell markers present amongst genes representative of those two lineages, a significantly increased basal and decreased secretory cell fraction in polyp tissues relative to healthy controls is identified (FIG. 15c,d). This mirrors the findings from the cohorts between non-polyp and polyp tissues, highlighting that the changes in cellular composition observed also typify the divergence between the healthy and polyp states. Taken together, these data suggest that nasal polyps, relative to both healthy and CRS non-polyp tissue, are comprised of a barrier with diminished function amongst secretory cells (FIG. 3d,e), an almost complete loss of glandular cells, and an enhanced basal cell state throughout (FIG. 4i).

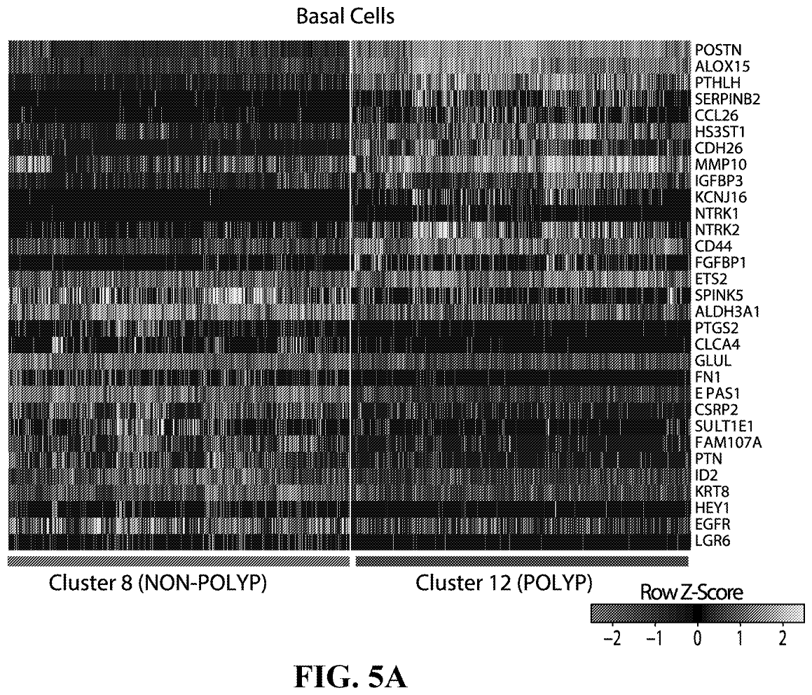

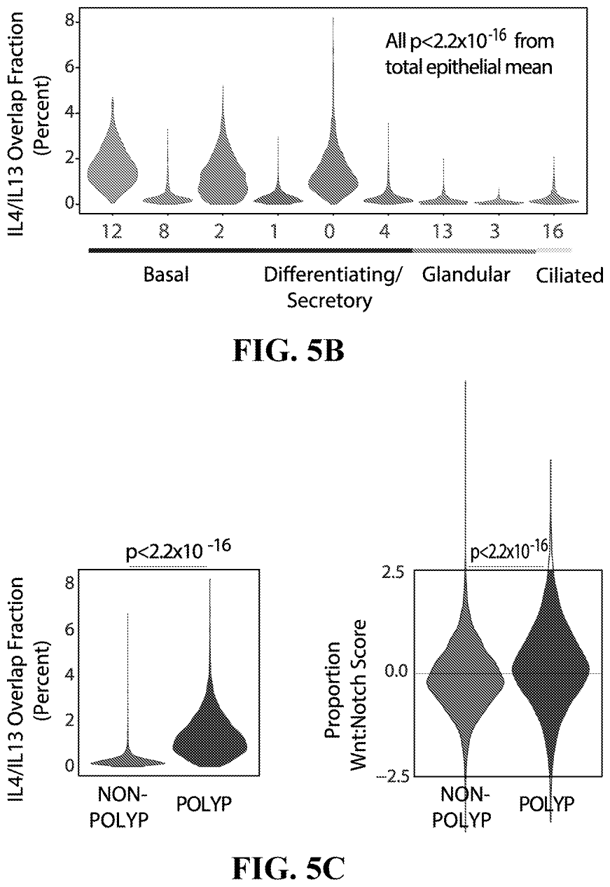

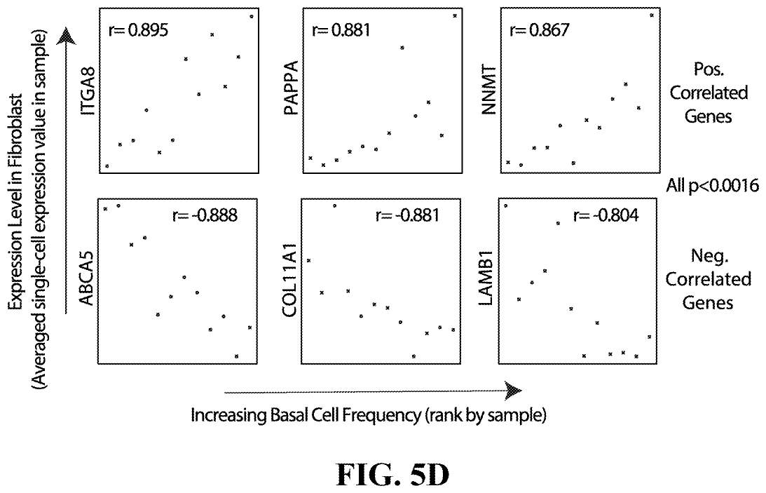

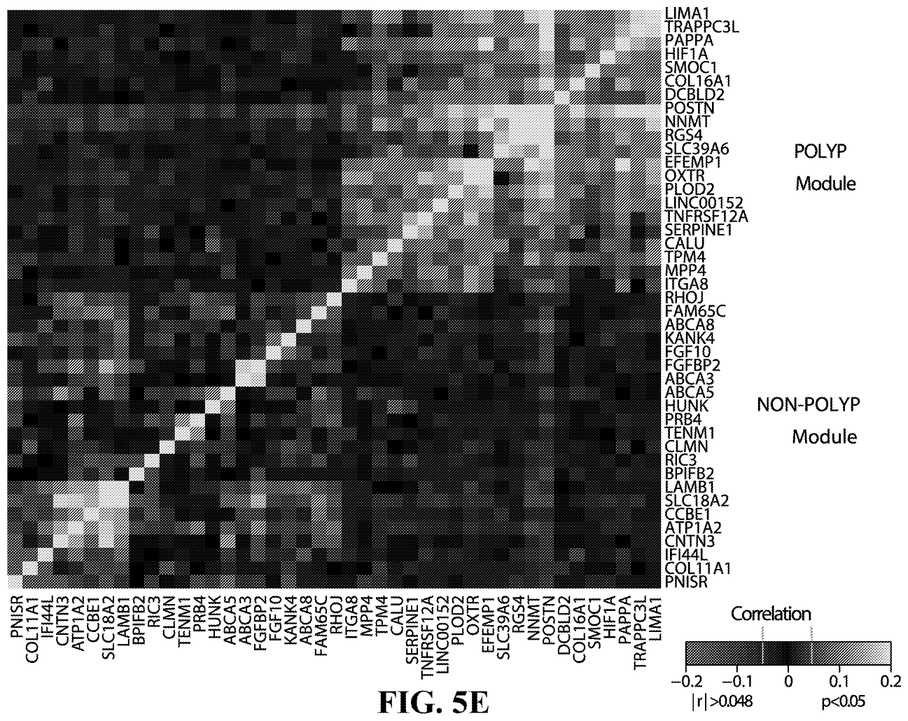

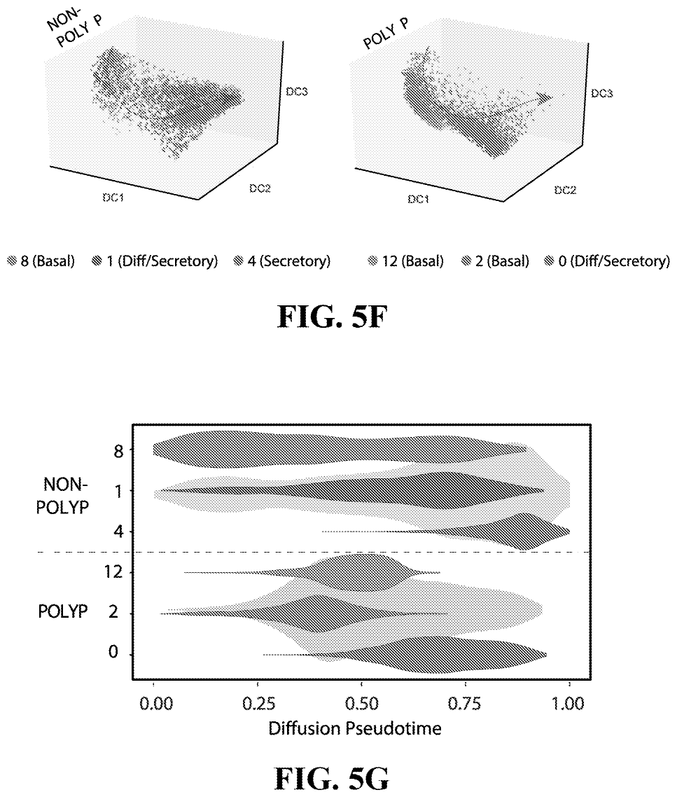

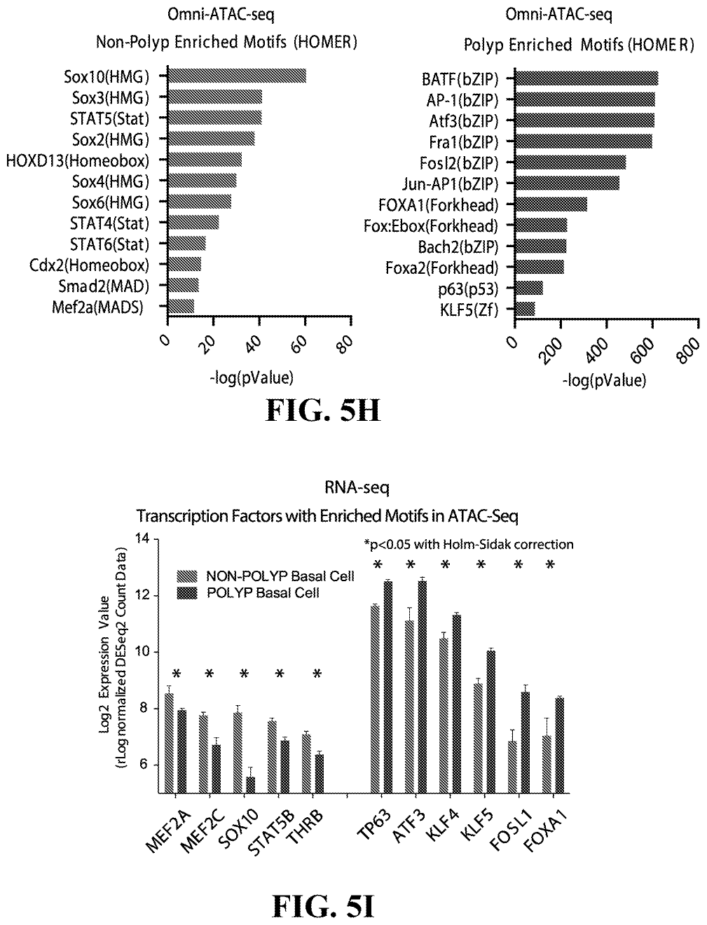

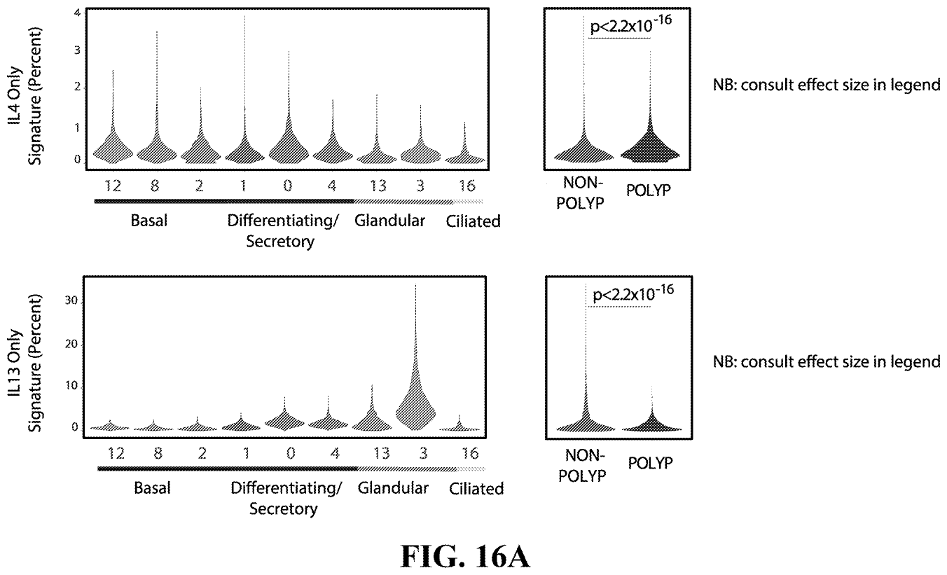

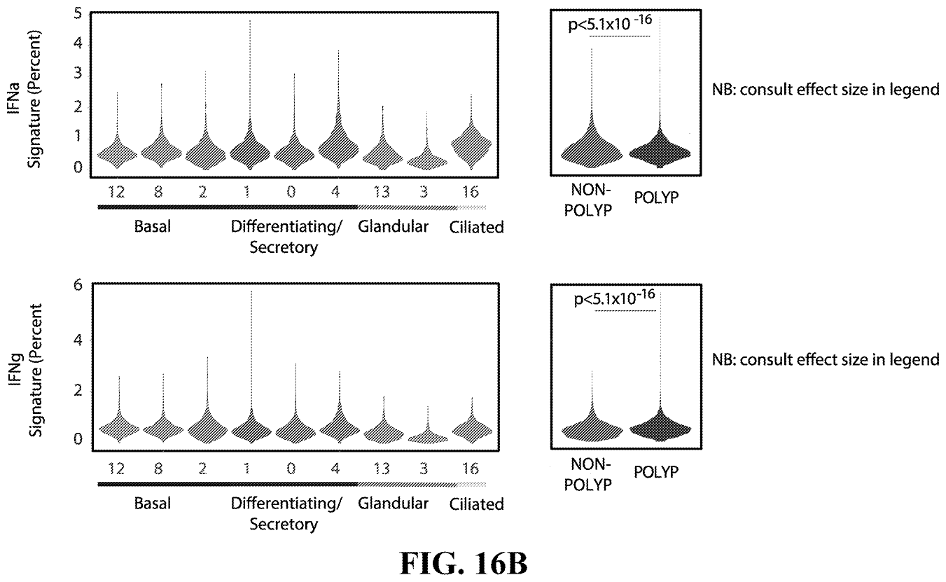

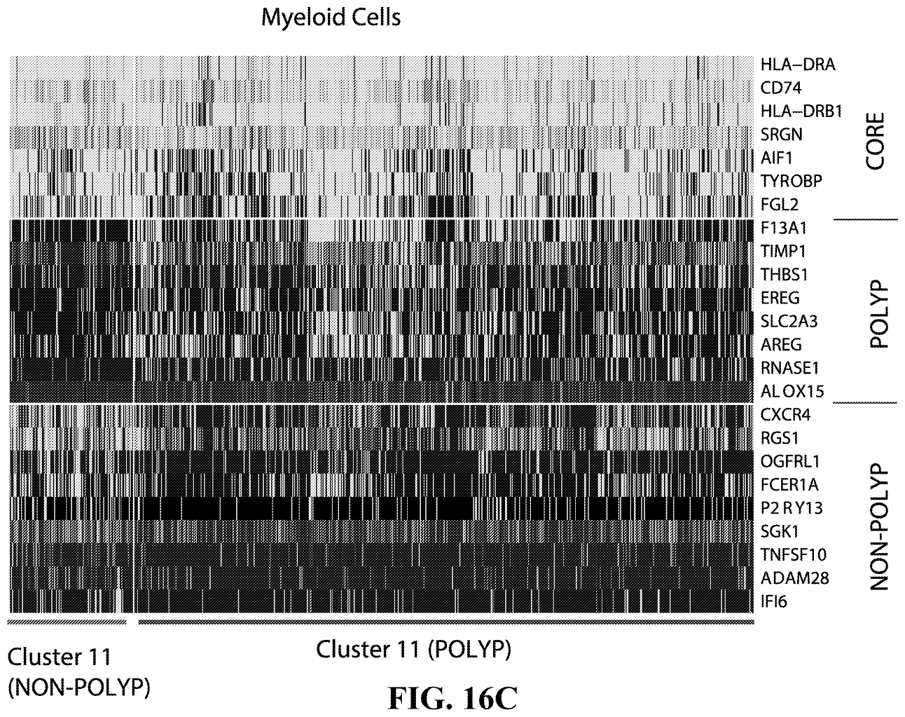

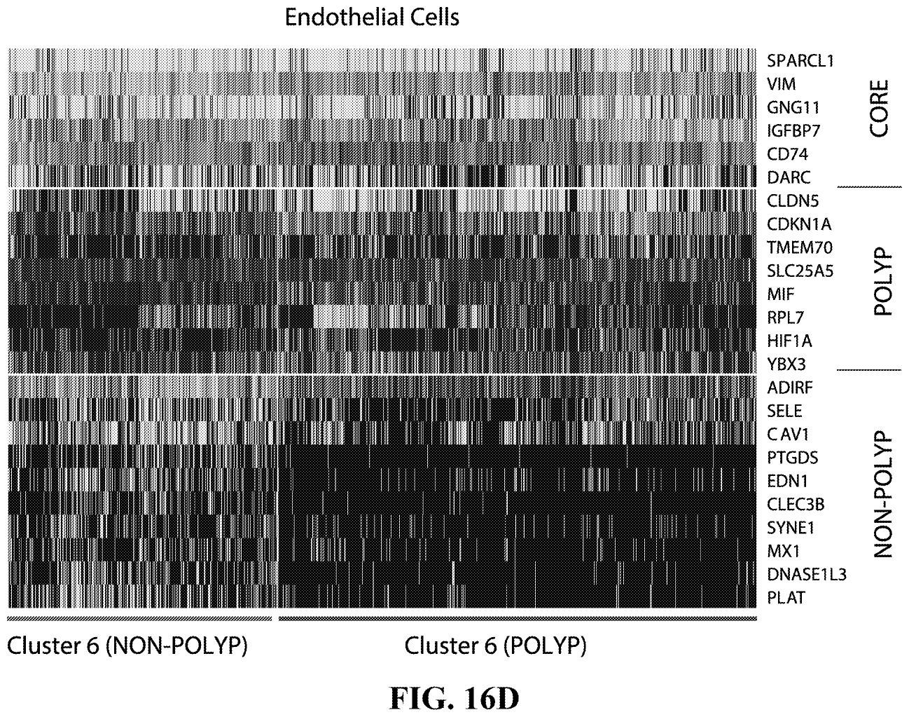

Next, it was sought to understand what mechanisms might account for the decreased epithelial diversity in polyps. Basal cells are the stem cells for upper airway respiratory epithelium^{3,14}, though evidence suggests that in severe injury, trans- and de-differentiation may also account for tissue repair⁸⁹. By comparing the transcriptomes of ethmoid sinus basal cells between the polyp and non-polyp ecosystems (FIG. 5a), elevated polyp expression of a set of transcripts-including POSTN, PTHLH, ALOX15, SERPINB2, HS3ST1, CDH26, MMP10 and CCL26-involved in extracellular matrix remodeling and chemo-attraction of effector cells^{79,116}, along with a decrease in protease inhibitor expression (SPINK5)¹¹⁷ and metabolic genes (ALDH3A1, CLCA4, GLUL) were identified (FIG. 5a). As several of these genes are known to be IL-4/IL-13 responsive¹¹¹, and IL-4/IL-13 targets were enriched in ethmoid sinus secretory cells (FIG. 3f), gene sets induced by these cytokines across basal and other epithelial subsets were more globally examined. It was determined that a combined IL-4/IL-13 signature (FIG. 5b,c; FIG. 16a) is strongly induced not only in differentiated polyp epithelium, but also at the level of basal cells, and has a large effect size between disease states (FIG. 5c). Conversely, Type-I IFN and IFNγ signatures-indicative of a Type-1 immune module^{18,111}—have small effect sizes (FIG. 16b; Methods). Furthermore, from specific hallmark genes, it was observed that the balance of Wnt (CD44) and Notch (HEY1) signaling, canonically linked to cellular differentiation¹¹⁸, appears altered in epithelium from polyps in favor of Wnt (FIG. 5a). This was further confirmed across full gene sets for these pathways (FIG. 5c)^{87,88,119}. Here, it is hypothesized that the persistence of such aberrant signaling may contribute to failed basal cell differentiation.

It was next sought to contextualize the basal cell findings related to extracellular matrix remodeling within their larger cellular ecosystems by asking whether cells which compose the basal cell niche, such as fibroblasts⁴⁸, were altered in polyps potentially contributing to basal cell dysfunction. Thus, averaged gene-expression values in single-cell fibroblasts whose expression correlated with basal cell frequency were looked for (FIG. 5d, FIG. 14b; all genes: abs(r)>0.7651, p<0.0037). Clustering over fibroblast genes that either positively or negatively correlated with basal cell frequency in the single-cell data identified a polyp-enriched gene module in fibroblasts associated with ITGA8, a hallmark gene of these cells during development of new airways but also fibrosis (FIG. 5e)^{120,121}. Significant changes in gene expression within myeloid and endothelial cells are also identified (FIG. 16c,d; FIG. 1b) by non-polyp and polyp disease state suggesting further alterations to the tissue microenvironment. It is noted that despite gene expression changes within all cell types recovered by disease state, the most striking changes observed comparing across disease within specific cell types were those seen in epithelial cells (FIG. 2d). Taken together, these data suggest that basal cells harbor an imprint of IL-4/IL-13 signaling, are found amongst an altered niche constituent, and a broadly altered microenvironment.

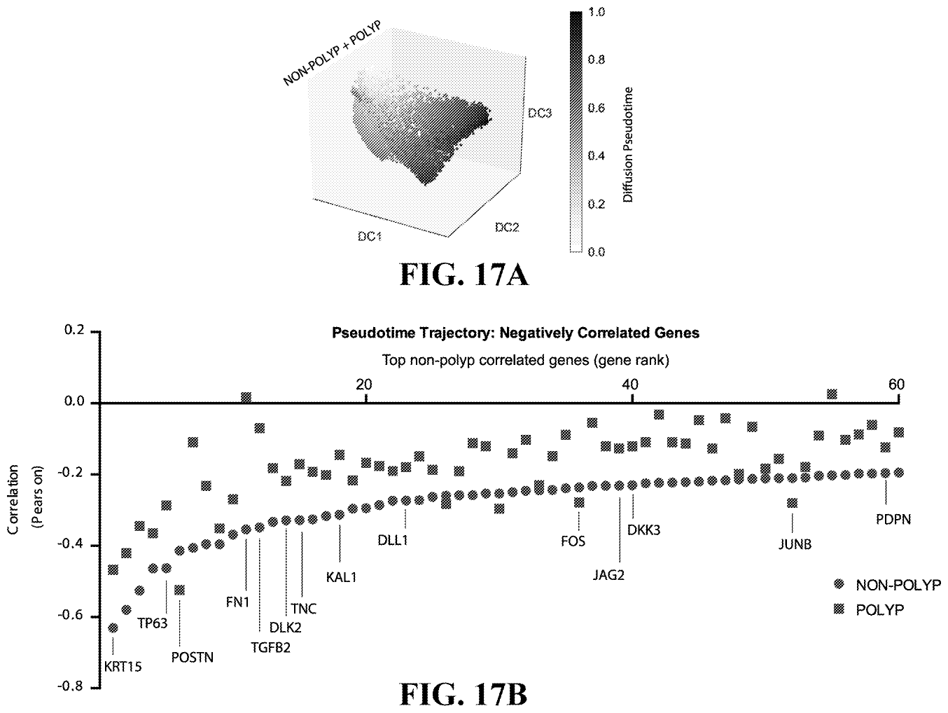

While a fundamental loss of glandular and ciliated cells in polyps was identified, substantial (but functionally altered) numbers of differentiating/secretory cells were recovered (FIG. 3e). Given this and the aforementioned differences in Wnt and Notch signaling, it was asked if by reconstructing how basal cells differentiate into mature secretory cells within non-polyp or polyp tissue, insight could be gained into the mechanisms leading to the observed loss in cellular diversity and function. Using diffusion pseudotime¹²² mapping, which seeks to provide the most likely reconstruction for the developmental progression of a set of cells (Methods), a trajectory was built for cells within basal and differentiating/secretory epithelial clusters (non-polyp clusters: 8-basal, 1-differentiating/secretory, 4-secretory; and polyp clusters: 12-basal, 2-basal, 0-differentiating/secretory; running several iterations starting from a random seed cell in cluster 8), over the combined basal and apical marker gene list (FIG. 5f; FIG. 17a). By calculating a pseudotime trajectory for cells from both non-polyps and polyps together, it could be asked where cells from each disease state fall along a shared inferred temporal axis (FIG. 5f,g, FIG. 17a). In the non-polyp ecosystem, it was observed that basal cells (cluster 8) traverse a much wider swath of common pseudotime, with the majority of cells distributed towards the end of the trajectory (cluster 4; FIG. 5f,g, FIG. 17a). Conversely, in polyps, basal cell clusters 2 and 12 accumulate shy of the midpoint of the trajectory, appearing to lose the true progenitor position occupied by cluster 8, and yet failing to productively contribute to later differentiation states (FIG. 5f,g, FIG. 17a). Furthermore, cluster 0 does not reach the terminally differentiated state observed in cluster 4, suggesting that the aforementioned expression differences (FIG. 5d,e) may, in part, be due to a failure of terminal differentiation. Ordering the cells according to this common axis, the full gene expression matrix was examined in order to identify which genes may become dysregulated in polyps during differentiation. It was found that: 1) DLK2, DLL1, JAG2, DKK3 (mediators of Wnt and Notch signaling, FIG. 5c) and 2) POSTN, FN1, TNC (extracellular matrix components, FN1 and TNC are ligands for ITGA8, FIG. 5e) were both significantly negatively correlated with pseudotime in non-polyps and had altered correlations in polyps (based on abs(Fisher's Z)>3.8; FIG. 17b, ), suggesting important contributions of cell-extrinsic developmental pathways and matrix-interacting receptors in regulating basal cell fate.

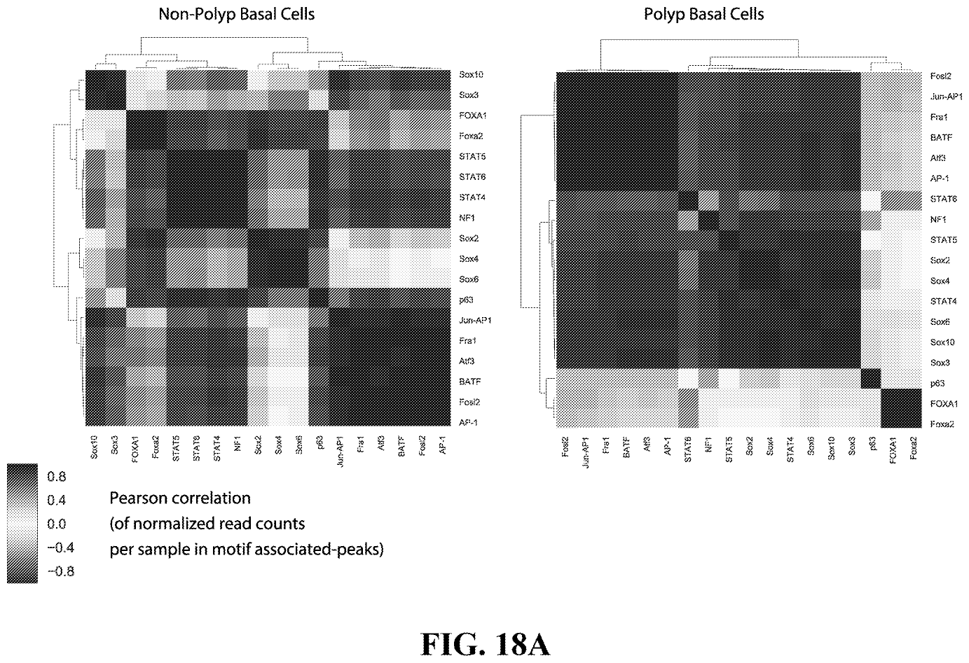

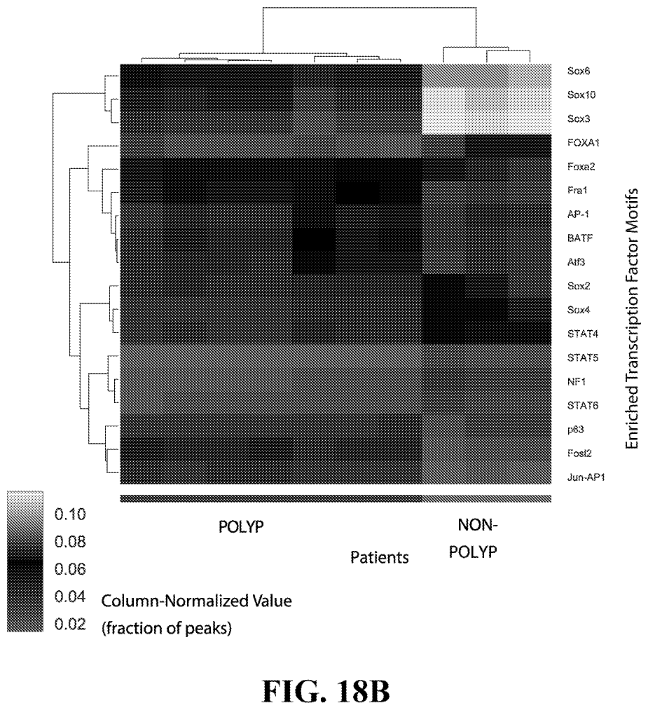

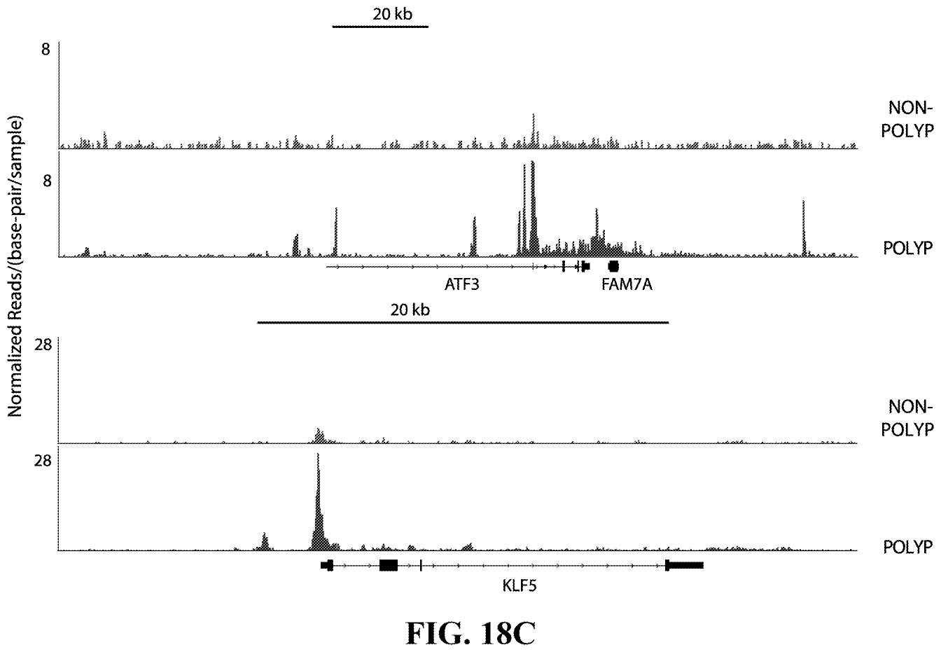

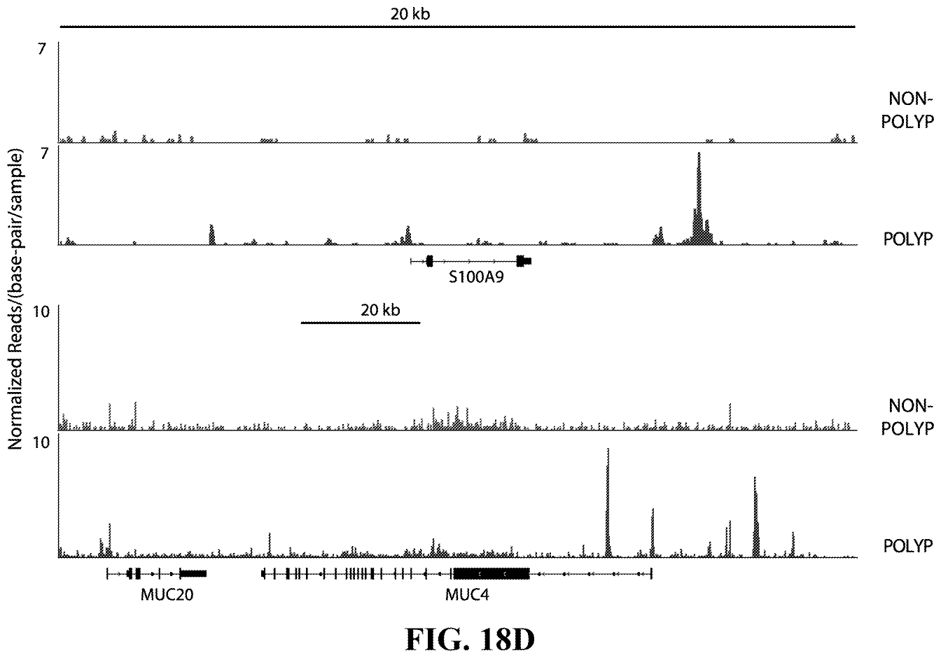

As these data highlighted an impairment in differentiation of basal cells in polyp tissue, basal cells were next sorted (FIG. 15) from 4 non-polyp and 7 polyp tissues (3 non-polyp and 7 polyp retained through data quality filtering) and performed Omni-ATAC-Seq to profile chromatin accessibility to search for the integration of extrinsic cellular signaling events resulting in intrinsic epigenetic changes^{123,124} (Methods). Chromatin can exist in several states within stem cells¹²⁵, including a poised state¹²⁶, providing a form of epigenetic memory^{127,128}, with influence on the propensity to differentiate towards particular cell fates. Among these data, it was identified that polyp basal cells had an enrichment in peaks, indicative of more accessible chromatin, (Methods) for bZIP transcription factor target motifs¹²⁹, including various AP-1 family members^{21,130} such as JUN, FOXA1¹³¹, ATF3¹³², KLF5¹³³ and p63¹³⁴ itself (NB: TP63 and JUNB were significantly negatively correlated with a productive differentiation trajectory in non-polyps, with a deviation observed in polyps (FIG. 5h; FIG. 17b; FIG. 18;). These proteins have previously been associated with the maintenance of an undifferentiated state, chromatin opening, and oncogenesis¹³⁵ Conversely, accessible motifs in non-polyp basal cells were enriched for Sox10, Sox4, Sox2, implicated in stemness, early progenitor cell differentiation programs, and lung branching morphogenesis¹³⁶, as well as STAT6 involved in allergic inflammation¹⁶, and Mef2-family transcription factors which bias towards specialized cell types within tissues¹³⁷ (FIG. 5h). Clustering of enriched motifs revealed Sox2/Sox4 and Sox3/Sox6/Sox10 modules enriched in non-polyp tissue (FIG. 18a,b). Furthermore, in polyp basal cells, p63 shows reduced correlation with the Sox and AP-1 modules, suggesting that higher order changes in chromatin structure may affect the activity of this defining basal cell transcription factor in severe disease¹²⁵ (FIG. 18). As p63 is essential for the proliferative potential of stem cells in stratified epithelia, yet largely dispensable for commitment and differentiation during development¹³⁸, it will be of interest to further understand the contextual activity of p63 in distinct types of tissue remodeling.

As motif enrichment analysis of accessible chromatin illustrates the potential for activity of closely related transcription factors¹²⁹, to refine which putative factors may be responsible for the differences Applicants observed, the ATAC-seq findings were confirmed and extended by performing low-input bulk RNA-seq on sorted basal cells from these same patients. In non-polyp basal cells, MEF2A, MEF2C, SOX10, STAT5B, and THRB are significantly upregulated transcription factors corresponding with enriched motifs (FIG. 5h,i). In polyp basal cells, increased expression of TP63, ATF3, KLF4, KLF5, FOSL1, and FOXA1 is found (FIG. 5h,i). Intriguingly, transcription factors such as ATF3 and KLF5 showed increased accessibility in the promoters and regions adjacent to the genes themselves raising the possibility that the transcription factor networks associated with basal cell hyperplasia could persist in the absence of inflammatory triggers, or be recalled more rapidly in their presence (FIG. 18c). Taken together, these basal cell gene signature, differentiation trajectory analyses and epigenetic studies led to the prediction that during chronic T2I, basal cell differentiation is intrinsically impaired through the influence of extrinsic cues (e.g., IL-4/IL-13 and Wnt pathway). This results in a barrier with reduced cellular and functional diversity, akin to an ecosystem with compromised biodiversity, and consequently an overall reduction in tissue health¹¹².

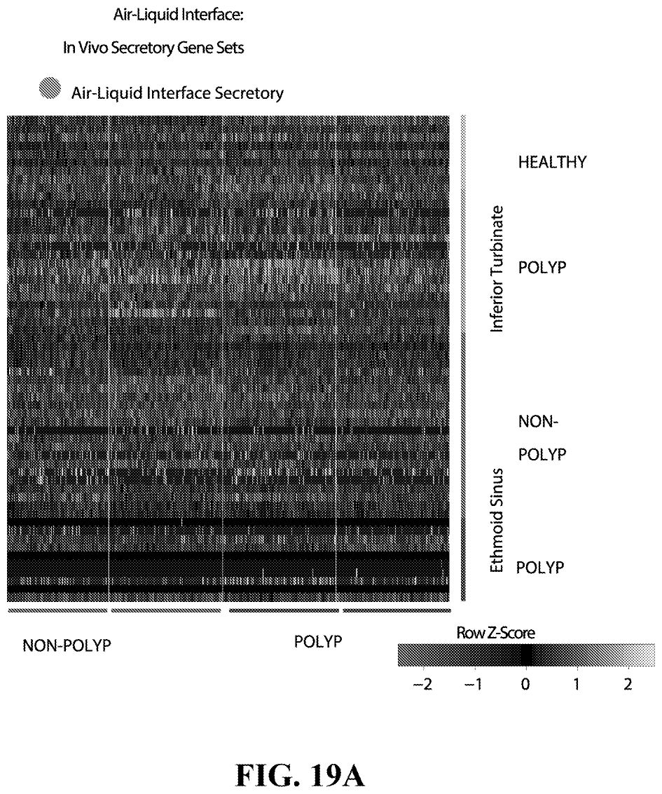

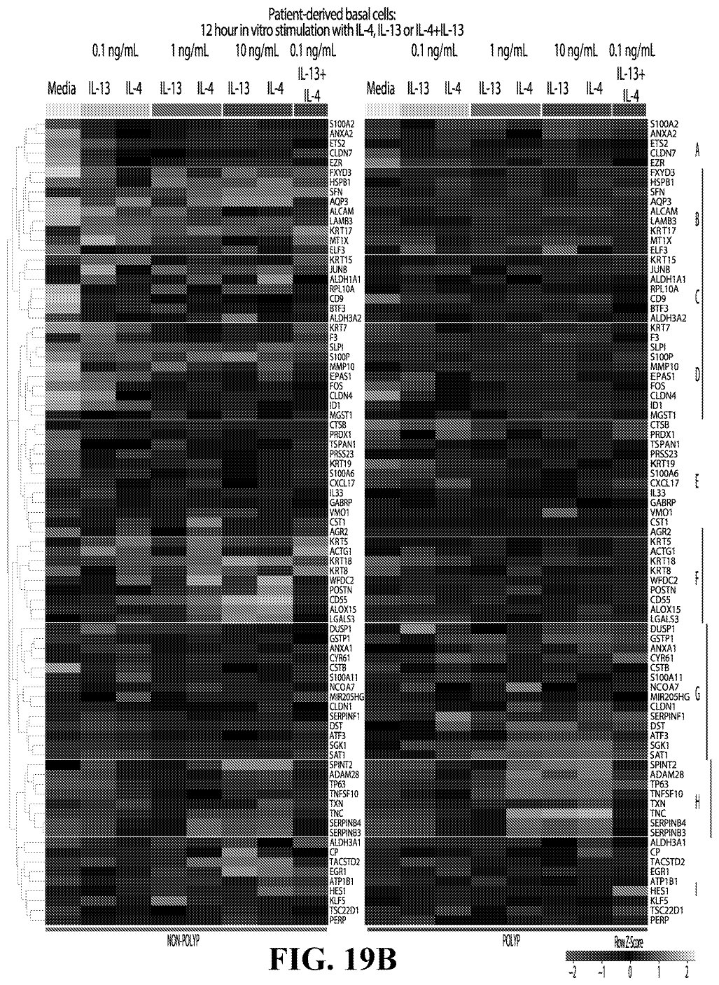

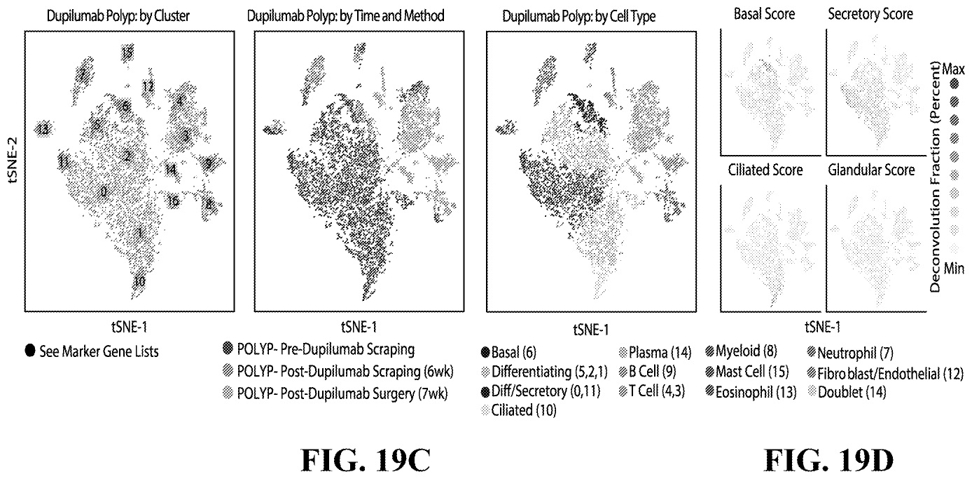

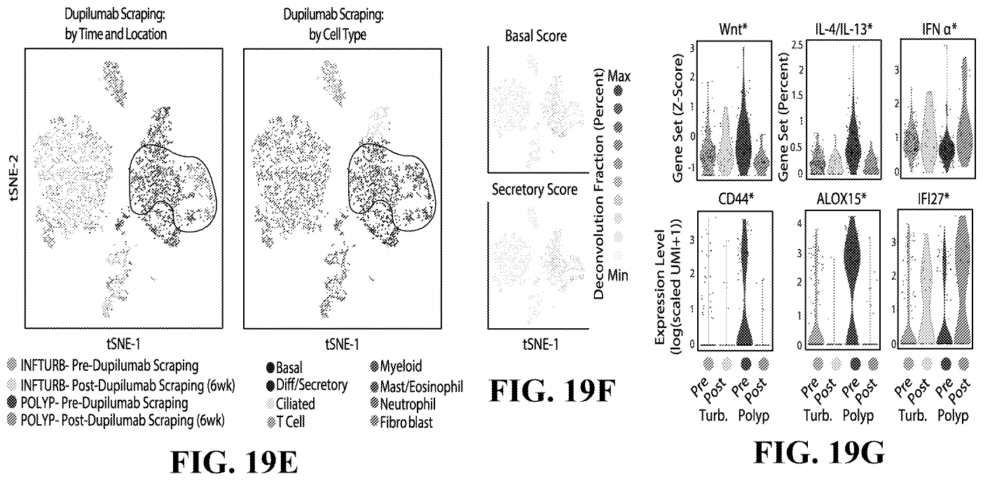

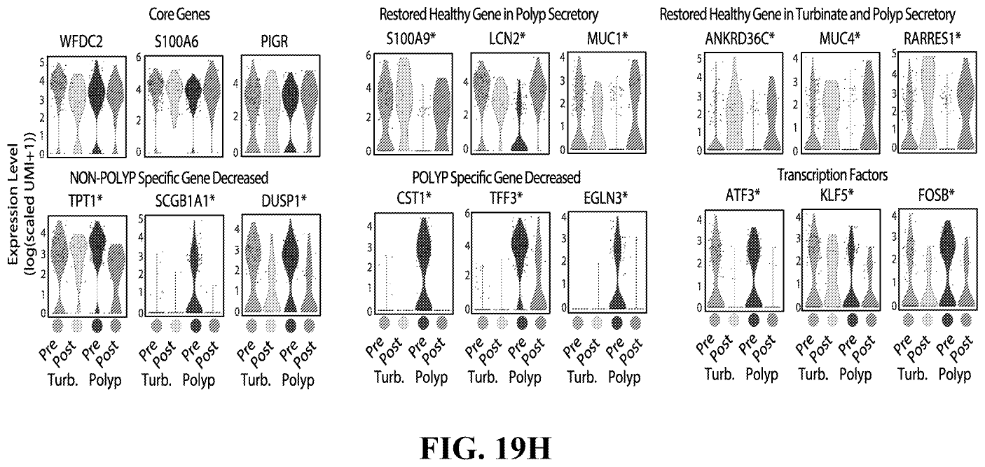

To functionally test whether polyp basal cells maintain an intrinsically altered differentiation potential in vitro, basal cells from non-polyp and polyp tissue ex vivo are expanded and seeded into air-liquid interface (ALI) cultures¹³⁹. After 21 days, these nasal cultures yield a differentiated and pseudostratified epithelium. The epithelial deconvolution gene lists are applied (Methods) to relate the cell types generated in ALI cultures to in vivo. Four main cell types were recovered including basal, secretory, ciliated, and a hybrid cell type expressing canonical transcription factors of both secretory (SPDEF¹⁴⁰) and ciliated (FOXJ1³¹) cells (FIG. 6a,b). A bias is noted in ALI cultures for producing a larger fraction of ciliated cells than what was recovered in vivo (compare FIG. 4a), and the appearance of hybrid ciliated and secretory cells suggesting a strong push towards terminal differentiation^{3,90}. As striking differences had previously been noted in the cell states of secretory cells from polyps relative to other tissues (FIG. 2e, FIG. 3e), the expression of in vivo secretory gene sets was assessed (FIG. 19a) in these ALI cultures, noting recovery of gene expression present in healthy inferior turbinate (S100A9, MUC4) and non-polyp sinus tissue (PSCA, SCGB1A1), with a complete absence of genes highly expressed in polyp secretory cells in vivo (CST1, POSTN) (FIG. 6c; FIG. 19a). Intriguingly, genes such as S100A9 and MUC4 have increased chromatin accessibility in basal cells from polyps relative to non-polyps (FIG. 18d), suggesting that if basal cells could be “released” from their trapped state, that expression of these gene products might occur in vivo.

As CST1 and POSTN, canonical IL-13 induced genes^{111,141}, were not detected in ALI cultures, it was hypothesized that the addition of IL-13 might reveal a defect in basal cell differentiation. This was tested by addition of a range of IL-13 concentrations to non-polyp or polyp-derived ALI cultures, and performed flow cytometry as in (FIG. 4d; FIG. 15b) to assess the relative ratio of basal to differentiated epithelial cells (FIG. 6d). While the addition of IL-13 did increase the ratio of basal to differentiated cells (*p<0.0224, 2-way ANOVA of IL-13 dosage; Methods), it did not act to preferentially inhibit differentiation in polyp-derived cultures at any of the doses tested (FIG. 6d). Taken together, these data suggest that basal cells from polyps have the potential to differentiate towards a mixed-tissue secretory cell phenotype observed in vivo (FIG. 19a), if provided with strong and sustained extrinsic cues, even in the presence of IL-13. It will be important to formally assess if the relevant clinical features of barrier tissue diseases are maintained ex vivo in relevant organoid systems.

As ALI cultures appear to enforce strong terminal differentiation on basal cells, it was directly tested how IL-4, IL-13, or both, act to induce rapid (12 hour) expression of genes in basal cells with the hypothesis that polyp basal cells would respond more vigorously and/or to lower doses of exogenous cytokines¹⁴². Surprisingly, when low-input bulk RNA-seq was performed and differential expression testing of genes induced by cytokine treatment on basal cells from non-polyp and polyp tissue cultured ex vivo, 482 genes were identified induced in non-polyp basal cells, whereas only 42 were significantly induced above baseline in polyp basal cells (FIG. 6e; Bonferroni corrected p<0.05 Table 16). A PCA plot over the most variable genes highlighted that while non-polyp basal cells cultured in the absence of exogenous cytokines were grouped together, those from polyp basal cells were found distributed near samples which had been stimulated with cytokine (FIG. 6e). Identifying overlaps in genes significantly induced by cytokine treatment in non-polyp basal cells with genes upregulated in polyp vs. non-polyp basal cells, it was noted that 132 cytokine-induced genes in non-polyp basal cells were already induced in polyp basal cells at baseline (FIG. 6e; Bonferroni corrected p<0.05 Table 16). To anchor the understanding of these differences in gene expression within the aforementioned in vivo changes in cellular differentiation, they were specifically examined over the list of genes used to generate pseudotime trajectories (FIG. 5f). It was noted that the basal cells from polyps had baseline decreased (and further decreased through the addition of cytokine) expression of sets of genes encompassing KRT15 and AQP3, both highly expressed in non-polyp basal cells in vivo (FIG. 19b). Importantly, this line of experimentation was performed in cells after 5 weeks of culture ex vivo, highlighting the robust nature of the persistent expression of IL-4/IL-13-induced genes in basal cells from polyps¹⁴³

The overlap of genes upregulated in polyp basal cells relative to non-polyp basal cells at baseline, induced in polyp basal cells above baseline by cytokine, and induced in non-polyp basal cells by cytokine was focused on next. This resolved two genes, of which one was CTNNB1 (p3-catenin) the key effector of Wnt pathway activation¹¹⁸. The previously unappreciated findings are highlighted from the data that CTNNB1 was robustly expressed in non-polyp and polyp basal cells in a dose-sensitive fashion to IL-4 and IL-13, that polyp basal cells already expressed CTNNB1 at the levels which are induced by cytokine treatment of non-polyp basal cells, and that gene expression was significantly increased across the entire dose range tested between disease categories (FIG. 6f). Furthermore, Wnt-pathway target genes were significantly upregulated across the doses tested confirming functional activation of the pathway (FIG. 6f). The increased expression of CTGF in polyp basal cells at baseline and across several doses of cytokine is also highlighted, as this growth factor is both a Wnt/P-catenin-regulated gene and an extracellular regulator of the pathway^{22,144} (FIG. 6f). Intriguingly, a recent study suggested that exogenous Wnt provided to ALI cultures led to morphological changes in cells resembling basal cell hyperplasia¹⁴⁵. Furthermore, the Wnt pathway is a key regulator of epithelial-mesenchymal transition¹⁴⁶ (EMT), which has been identified as a key element of barrier dysfunction in nasal polyps and other type 2 inflammatory diseases². Based on the increased IL-4/IL-13 and Wnt target gene signatures observed in epithelial cells from polyps (FIG. 5c), and the functional test for IL-4/IL-13 induced genes which persist in basal cells from polyps, it is proposed that chronic IL-4/IL-13 exposure in vivo can lead to persistent expression of CTNNB1 and Wnt target genes by basal cells in a cell intrinsic fashion, even in the absence of exogenous cytokine.

While these ex vivo experiments allowed for assessment of the specific baseline and induced IL-4/IL-13 gene signature of basal cells in vitro, an opportunity was sought through which to validate the in vivo relevance of the observational, mechanistic and functional data on how allergic inflammatory cytokines influence basal cell states. Fortuitously, one of the nasal polyp patients sampled in the nasal scraping study commenced treatment with dupilumab, a monoclonal antibody (mAb) targeting the shared IL-4Ra subunit of the IL-4 and IL-13 receptors¹⁴⁷, to treat atopic dermatitis, allowing for the test of the collateral effects of the medication on nasal polyp ecosystem (FIG. 6g; FIG. 19c,d). The cells recovered from a pre-treatment scraping were compared to those acquired through scraping 6 weeks later, and through a surgical intervention 7 weeks after the initiation of therapy (FIG. 6g; FIG. 19c,d). While the distinct nature of the sampling methods and limited sample size preclude a formal analysis of basal to differentiated cell ratios, the power of the scRNA-Seq data was leveraged to identify clusters of the same cell type in order to look within relatively homogenous populations for treatment induced changes in gene expression (FIG. 6g; FIG. 19c,d; NB given the unique nature of the in vivo validation, the reporting of data is limited to differentially expressed genes which were previously contextualized in the manuscript through in vivo and/or ex vivo analyses, with a focus on basal and secretory cells).

To compare changes in gene expression after IL-4/IL-13 receptor blockade, the cluster with the highest basal cell score was identified (FIG. 19c,d). This cluster contained cells from both the pre-dupilumab scraping and the post-dupilumab surgery (FIG. 6g,). A heatmap was then generated containing the top marker genes for the basal cell cluster, agnostic to treatment status, followed by genes significantly differentially expressed between pre- and post-dupilumab samples (FIG. 6h). The myeloid cell clusters present in both conditions were leveraged in order to highlight genes whose expression may be decreased through cytokine blockade across multiple cell types in the environment, and those changes restricted to basal cells (FIG. 6i). Contextualizing these findings within the previous data, several key gene sets were identified, including a conserved core set of basal cell genes (TP63, CLDN1, ITGA2) (FIG. 6j) unchanged by treatment. Intriguingly, transcription factors upregulated in polyp basal cells identified through omni-ATAC-seq and low-input RNA-seq (ATF3, KLF5, FOSB) were significantly decreased by treatment^{130,132,133,148} (FIG. 6j). While Wnt pathway target gene expression was globally reduced (Wnt pathway gene set, 1D2), CTNNB1 expression was notably retained (FIG. 6j) as might be predicted from the persistence observed for the latter in the in vitro data. However, several genes upregulated at baseline in ex vivo polyp basal cells were significantly decreased, including CTGF and SERPINE1 (FIG. 6j). Based on the previous analysis of non-polyp vs. polyp basal cells in vivo, POSTN, NTRK2 and HS3ST1 were identified, amongst others, as significantly upregulated in polyps whereas here conserved expression of POSTN and NTRK2 is noted, with diminished expression of HS3ST1 (FIG. 6j). Lastly, for genes that were upregulated both in vitro and in vivo in polyp basal cells (TNC, SOX4, PTHLH), none were significantly decreased by IL-4/IL-13 blockade (FIG. 6j), suggesting that some genes in this patient, and at this timepoint sampled, persist.

Since a mAb targeting IL4Ra (shared IL-4/IL-13 receptor) is used clinically (dupilumab), Applicants identified a “residual” signature to target in a combination therapy. Applicants compared the in vivo basal polyp vs non-polyp gene expression differences (FIG. 5a, Table 15) and overlapped them with the Pre- vs Post-dupilumab treatment (FIG. 6H and Table 18). A Venn diagram was constructed (FIG. 20 and Table 17). The 68 genes are genes downregulated by treatment that could be used as biomarkers to track treatment efficacy. The 382 genes are downregulated by treatment, but may not be specifically polyp related.

TABLE 18
This sheet contains genes displayed in FIG. 6H, differential expression
in basal cells by bimodal test: Pre vs Post-dupilumab

| | p_val | avg_diff | pct. 1 | pct. 2 |
|---|---|---|---|---|
| ATF3 | 3.98E−42 | 1.943554766 | 0.764 | 0.185 |
| SYF2 | 2.96E−28 | 1.58726515 | 0.704 | 0.192 |
| ALDH3A1 | 4.91E−28 | 1.515908373 | 0.758 | 0.298 |
| DUSP1 | 4.12E−31 | 1.413573799 | 0.803 | 0.305 |
| SPAG4 | 7.58E−15 | 1.381978719 | 0.293 | 0.013 |
| NCOA7 | 7.02E−19 | 1.338883563 | 0.618 | 0.199 |
| CTGF | 3.73E−11 | 1.336221395 | 0.325 | 0.066 |
| ZFP36 | 6.35E−25 | 1.318508174 | 0.723 | 0.258 |
| NR4A1 | 5.08E−22 | 1.234060396 | 0.618 | 0.146 |
| CYR61 | 2.26E−15 | 1.231779922 | 0.452 | 0.086 |
| HSPA1A | 1.21E−13 | 1.189486774 | 0.561 | 0.212 |
| BTG2 | 1.54E−17 | 1.170524664 | 0.605 | 0.185 |
| MSMB | 3.55E−11 | 1.166878564 | 0.312 | 0.046 |
| IL8 | 9.83E−06 | 1.166196469 | 0.213 | 0.046 |
| TFF3 | 3.11E−15 | 1.16388623 | 0.481 | 0.106 |
| SUOX | 2.50E−13 | 1.124738452 | 0.226 | 0 |
| CLDN4 | 6.54E−16 | 1.122971818 | 0.586 | 0.185 |
| EGR1 | 1.81E−25 | 1.072700973 | 0.803 | 0.318 |
| GLUL | 1.13E−21 | 1.033834286 | 0.822 | 0.464 |
| ANXA1 | 1.10E−08 | 1.028926551 | 0.615 | 0.371 |
| EGR3 | 8.63E−10 | 1.009294683 | 0.22 | 0.013 |
| JUN | 1.74E−31 | 1.004047883 | 0.933 | 0.629 |
| ID1 | 1.77E−12 | 0.969850631 | 0.618 | 0.291 |
| LMNA | 5.54E−11 | 0.965792444 | 0.481 | 0.159 |
| JUNB | 2.53E−16 | 0.961758417 | 0.745 | 0.391 |
| KRT17 | 6.77E−14 | 0.956465476 | 0.666 | 0.344 |
| HSPA1B | 3.60E−12 | 0.947133624 | 0.497 | 0.152 |
| PPP1R15A | 8.35E−17 | 0.941729535 | 0.688 | 0.291 |
| TSC22D1 | 2.54E−12 | 0.930585896 | 0.646 | 0.364 |
| SERPINE1 | 5.26E−07 | 0.921453296 | 0.303 | 0.086 |
| ETS2 | 5.90E−17 | 0.902193626 | 0.739 | 0.377 |
| DNAJB1 | 3.41E−14 | 0.895212028 | 0.624 | 0.291 |
| MTSS1L | 6.57E−09 | 0.890132224 | 0.325 | 0.073 |
| ERRFI1 | 2.24E−08 | 0.887134043 | 0.354 | 0.106 |
| KRT5 | 2.03E−20 | 0.885945883 | 0.841 | 0.51 |
| TIPARP | 3.78E−11 | 0.884103241 | 0.414 | 0.106 |
| LGALS3 | 2.82E−11 | 0.883325857 | 0.573 | 0.238 |
| SERTAD1 | 1.64E−09 | 0.879261008 | 0.35 | 0.093 |
| TSC22D3 | 1.02E−08 | 0.879189839 | 0.414 | 0.139 |
| PTP4A1 | 9.95E−08 | 0.864282078 | 0.366 | 0.119 |
| BPIFB1 | 3.71E−07 | 0.846717456 | 0.255 | 0.053 |
| MAFF | 3.27E−08 | 0.839729805 | 0.274 | 0.053 |
| MUC5AC | 1.77E−08 | 0.83649186 | 0.178 | 0.007 |
| FOSB | 2.88E−17 | 0.834946437 | 0.866 | 0.55 |
| PMAIP1 | 6.25E−07 | 0.831613849 | 0.373 | 0.139 |
| HSPA8 | 8.26E−12 | 0.831382548 | 0.621 | 0.272 |
| ZFP36L2 | 5.30E−08 | 0.829999424 | 0.538 | 0.278 |
| IER2 | 4.99E−11 | 0.828242 | 0.592 | 0.252 |
| NFKBIA | 1.42E−08 | 0.823393618 | 0.481 | 0.205 |
| CDKN1A | 7.30E−08 | 0.821263144 | 0.28 | 0.06 |
| BRD2 | 2.39E−11 | 0.818183773 | 0.592 | 0.245 |
| RPS16 | 7.46E−13 | 0.804293112 | 0.707 | 0.417 |
| CAPS | 1.28E−06 | 0.798191527 | 0.271 | 0.073 |
| EZR | 1.41E−12 | 0.795582199 | 0.761 | 0.437 |
| FAM107B | 7.72E−08 | 0.794241709 | 0.411 | 0.152 |
| KRT19 | 1.83E−21 | 0.791248207 | 0.936 | 0.715 |
| F3 | 4.35E−15 | 0.785408094 | 0.841 | 0.603 |
| KLF4 | 5.51E−13 | 0.772145253 | 0.631 | 0.258 |
| RHOB | 1.61E−06 | 0.762827191 | 0.411 | 0.172 |
| FOSL2 | 6.61E−09 | 0.761118577 | 0.22 | 0.033 |
| GPRC5A | 3.82E−05 | 0.759007225 | 0.21 | 0.053 |
| SOCS3 | 4.41E−06 | 0.75100327 | 0.344 | 0.126 |
| TRIB1 | 1.42E−09 | 0.745182054 | 0.264 | 0.04 |
| MIR22HG | 1.80E−05 | 0.738597823 | 0.242 | 0.066 |
| HSP90AA1 | 1.13E−11 | 0.732103122 | 0.764 | 0.43 |
| PRDX1 | 1.12E−10 | 0.729835608 | 0.688 | 0.371 |
| WEE1 | 2.11E−05 | 0.72963845 | 0.248 | 0.099 |
| FTL | 2.95E−10 | 0.72842054 | 0.755 | 0.536 |
| SLC25A25 | 5.74E−07 | 0.719579065 | 0.318 | 0.099 |
| VMP1 | 5.13E−05 | 0.715607454 | 0.373 | 0.172 |
| DDIT3 | 4.74E−06 | 0.71490177 | 0.191 | 0.053 |
| RND3 | 5.82E−06 | 0.714433674 | 0.443 | 0.212 |
| ATP5G2 | 3.58E−09 | 0.714010862 | 0.389 | 0.132 |
| TACSTD2 | 1.08E−10 | 0.705312006 | 0.764 | 0.49 |
| HBEGF | 3.02E−05 | 0.698912137 | 0.232 | 0.073 |
| CD55 | 7.61E−07 | 0.697661724 | 0.484 | 0.225 |
| EMP1 | 8.05E−06 | 0.696864934 | 0.261 | 0.073 |
| MCL1 | 5.39E−10 | 0.694262613 | 0.608 | 0.305 |
| CEBPD | 3.24E−07 | 0.691237721 | 0.322 | 0.093 |
| FKBP5 | 1.11E−07 | 0.691165328 | 0.191 | 0.02 |
| YPEL5 | 3.14E−06 | 0.689218583 | 0.268 | 0.073 |
| ACTG1 | 7.45E−14 | 0.688483839 | 0.793 | 0.57 |
| C12orf57 | 1.08E−06 | 0.661746995 | 0.36 | 0.126 |
| KAL1 | 3.91E−05 | 0.656830687 | 0.341 | 0.139 |
| RN7SK | 4.44E−06 | 0.648636791 | 0.277 | 0.086 |
| NR1D2 | 8.03E−07 | 0.64426285 | 0.21 | 0.033 |
| FXYD3 | 2.80E−10 | 0.644072952 | 0.758 | 0.457 |
| KLF5 | 3.04E−07 | 0.638405376 | 0.573 | 0.298 |
| FOS | 2.52E−16 | 0.635517655 | 0.933 | 0.854 |
| ANKRD37 | 0.000483262 | 0.635299401 | 0.226 | 0.079 |
| MIDN | 4.87E−08 | 0.635000437 | 0.318 | 0.086 |
| PER2 | 2.94E−06 | 0.63475876 | 0.376 | 0.146 |
| STARD7 | 0.000104524 | 0.633441354 | 0.242 | 0.079 |
| WFDC2 | 7.11E−06 | 0.631284597 | 0.443 | 0.205 |
| DUSP6 | 3.38E−05 | 0.630872124 | 0.376 | 0.172 |
| HSPB1 | 1.49E−07 | 0.628567363 | 0.586 | 0.318 |
| SEPW1 | 6.61E−05 | 0.626725773 | 0.344 | 0.152 |
| MYC | 2.30E−05 | 0.626211916 | 0.322 | 0.119 |
| CXCL17 | 2.79E−05 | 0.623334972 | 0.379 | 0.172 |
| KLF9 | 2.46E−05 | 0.623174952 | 0.191 | 0.04 |
| SYT8 | 5.26E−06 | 0.616103574 | 0.389 | 0.159 |
| GPX4 | 6.05E−06 | 0.610970451 | 0.545 | 0.325 |
| MIR24-2 | 1.26E−05 | 0.610862727 | 0.232 | 0.06 |
| EPS8 | 0.000119479 | 0.609937149 | 0.232 | 0.073 |
| AHNAK | 1.66E−07 | 0.60750904 | 0.815 | 0.675 |
| JUP | 0.000685626 | 0.605246417 | 0.217 | 0.093 |
| ADH7 | 4.61E−06 | 0.600545084 | 0.449 | 0.205 |
| S100P | 0.000571885 | 0.594858806 | 0.229 | 0.079 |
| POLR2A | 0.000112543 | 0.594512865 | 0.239 | 0.079 |
| PPP1R10 | 0.000467931 | 0.59369602 | 0.261 | 0.126 |
| GCNT1 | 0.000163933 | 0.591982182 | 0.159 | 0.04 |
| GAPDH | 1.85E−08 | 0.590813151 | 0.764 | 0.55 |
| IER3 | 2.17E−05 | 0.589338943 | 0.408 | 0.185 |
| HOOK1 | 0.000153073 | 0.583468726 | 0.277 | 0.106 |
| HN1L | 0.000619787 | 0.580399599 | 0.245 | 0.093 |
| TOMM20 | 2.81E−05 | 0.580220093 | 0.373 | 0.159 |
| SCGB1A1 | 4.90E−05 | 0.580122914 | 0.156 | 0.026 |
| GSR | 8.84E−06 | 0.577044274 | 0.261 | 0.079 |
| SERPINB5 | 0.005641052 | 0.575269478 | 0.229 | 0.106 |
| S100A6 | 6.99E−10 | 0.574140451 | 0.85 | 0.609 |
| PEBP1 | 1.84E−05 | 0.572454322 | 0.382 | 0.179 |
| CDC42EP4 | 0.000320839 | 0.571379566 | 0.178 | 0.053 |
| AQP3 | 9.38E−13 | 0.570018068 | 0.841 | 0.642 |
| ADRB2 | 2.08E−06 | 0.568353133 | 0.166 | 0.033 |
| DDIT4 | 2.12E−05 | 0.567047923 | 0.392 | 0.172 |
| PER1 | 0.000258812 | 0.565949309 | 0.194 | 0.053 |
| ADM | 2.00E−05 | 0.565879579 | 0.226 | 0.06 |
| IRF1 | 0.000537034 | 0.564726977 | 0.191 | 0.06 |
| PRDX6 | 4.58E−05 | 0.560724403 | 0.271 | 0.113 |
| RPS14 | 2.19E−09 | 0.556557382 | 0.901 | 0.762 |
| TPT1 | 1.24E−11 | 0.55521226 | 0.92 | 0.775 |
| PLXNB1 | 0.003130414 | 0.554486398 | 0.229 | 0.099 |
| BHLHE40 | 0.001071094 | 0.553394323 | 0.35 | 0.179 |
| SQSTM1 | 0.000145586 | 0.551995741 | 0.459 | 0.285 |
| SCP2 | 0.000169485 | 0.54381973 | 0.312 | 0.152 |
| KRT15 | 4.70E−06 | 0.542849293 | 0.675 | 0.45 |
| SGK1 | 0.000166405 | 0.539406687 | 0.452 | 0.272 |
| ACTB | 2.32E−06 | 0.538831801 | 0.672 | 0.43 |
| NABP1 | 0.007037741 | 0.538757053 | 0.197 | 0.086 |
| IGFBP3 | 0.002206778 | 0.536444711 | 0.338 | 0.179 |
| PPP1R15B | 2.11E−05 | 0.53378775 | 0.166 | 0.033 |
| SOD2 | 0.001809764 | 0.53353033 | 0.312 | 0.172 |
| SOX9 | 0.0058683 | 0.532817125 | 0.172 | 0.066 |
| CTSH | 8.75E−06 | 0.532284139 | 0.325 | 0.119 |
| MXD1 | 0.000176105 | 0.530904674 | 0.162 | 0.033 |
| THBS1 | 4.23E−05 | 0.528928093 | 0.299 | 0.119 |
| CIB1 | 0.003219596 | 0.526724059 | 0.274 | 0.132 |
| RAP2B | 0.000534322 | 0.526617865 | 0.204 | 0.073 |
| NEDD4L | 0.001429492 | 0.519949108 | 0.232 | 0.093 |
| RAB34 | 0.000182907 | 0.51960014 | 0.182 | 0.066 |
| PRDX5 | 0.000593622 | 0.514885313 | 0.468 | 0.298 |
| ANAPC11 | 0.001643405 | 0.514148097 | 0.268 | 0.119 |
| RAC1 | 0.002230942 | 0.513563297 | 0.245 | 0.113 |
| ARF1 | 0.000255819 | 0.511068751 | 0.357 | 0.166 |
| HS3ST1 | 0.000871777 | 0.509375157 | 0.334 | 0.166 |
| MAOA | 0.000971252 | 0.507597271 | 0.162 | 0.06 |
| HLA-E | 1.96E−06 | 0.505841822 | 0.64 | 0.377 |
| EID1 | 3.15E−05 | 0.50576594 | 0.519 | 0.285 |
| BAG5 | 0.001562896 | 0.504681231 | 0.185 | 0.06 |
| SERPINH1 | 0.001516112 | 0.503431843 | 0.197 | 0.066 |
| CITED2 | 0.00162453 | 0.503286082 | 0.201 | 0.086 |
| AJUBA | 6.42E−05 | 0.499489713 | 0.178 | 0.04 |
| CTSK | 0.003266871 | 0.498248642 | 0.172 | 0.06 |
| PAFAH1B2 | 0.00111898 | 0.497826302 | 0.178 | 0.053 |
| GNB2L1 | 2.64E−06 | 0.496972269 | 0.739 | 0.503 |
| LMO7 | 0.00121317 | 0.496593988 | 0.309 | 0.146 |
| PDPN | 0.000461599 | 0.492842695 | 0.201 | 0.06 |
| UBE3A | 0.014521505 | 0.492720719 | 0.182 | 0.093 |
| HNRNPAB | 0.000696455 | 0.492491021 | 0.194 | 0.066 |
| GSTP1 | 5.69E−06 | 0.492405148 | 0.65 | 0.411 |
| TM4SF1 | 0.017066446 | 0.491922533 | 0.22 | 0.106 |
| PIM3 | 1.50E−05 | 0.490723871 | 0.162 | 0.026 |
| DLX5 | 3.31E−05 | 0.489569249 | 0.201 | 0.046 |
| C9orf3 | 0.001699798 | 0.485287977 | 0.255 | 0.106 |
| HPGD | 0.008888546 | 0.484767434 | 0.178 | 0.079 |
| ANXA11 | 0.000144332 | 0.478098136 | 0.309 | 0.126 |
| UBC | 2.93E−06 | 0.475397578 | 0.768 | 0.563 |
| PLP2 | 0.00338649 | 0.475346176 | 0.236 | 0.099 |
| EGR2 | 2.48E−05 | 0.474580813 | 0.258 | 0.093 |
| S100A10 | 0.000501918 | 0.474129582 | 0.478 | 0.291 |
| LAMA5 | 0.001839223 | 0.470661441 | 0.306 | 0.152 |
| ALOX15 | 2.33E−05 | 0.470661205 | 0.726 | 0.523 |
| MUC16 | 0.046715344 | 0.469888638 | 0.229 | 0.126 |
| RPL4 | 5.16E−05 | 0.468684741 | 0.717 | 0.543 |
| TNS4 | 0.000359782 | 0.468007013 | 0.277 | 0.113 |
| TUBB | 0.0061683 | 0.467262158 | 0.239 | 0.146 |
| RHOC | 0.000634452 | 0.464925654 | 0.347 | 0.172 |
| CYP2S1 | 0.000229299 | 0.463052147 | 0.223 | 0.073 |
| PPAP2C | 0.010084627 | 0.462332512 | 0.197 | 0.086 |
| GPX1 | 0.000410565 | 0.461629759 | 0.309 | 0.146 |
| QARS | 0.003444918 | 0.461057559 | 0.236 | 0.099 |
| CSNK1D | 0.002393194 | 0.459519852 | 0.223 | 0.086 |
| HINT1 | 0.000485071 | 0.459003236 | 0.554 | 0.358 |
| PDLIM1 | 0.000313269 | 0.457964555 | 0.411 | 0.212 |
| BLVRB | 0.006916279 | 0.457228772 | 0.229 | 0.106 |
| PSMB6 | 0.000204104 | 0.455171363 | 0.159 | 0.033 |
| TSPO | 0.000124339 | 0.455044596 | 0.392 | 0.192 |
| CDCA7L | 0.000933716 | 0.454645143 | 0.166 | 0.046 |
| HNRNPF | 2.25E−07 | 0.452140821 | 0.366 | 0.132 |
| RPS4X | 2.72E−05 | 0.451980723 | 0.777 | 0.596 |
| EPB41L4A-AS1 | 0.007409298 | 0.451676007 | 0.182 | 0.073 |
| IRF6 | 0.001484145 | 0.451430318 | 0.29 | 0.132 |
| FSTL1 | 0.006060348 | 0.451009136 | 0.201 | 0.079 |
| FTH1 | 0.000522604 | 0.450787387 | 0.611 | 0.43 |
| UBL5 | 0.002969152 | 0.44767255 | 0.338 | 0.199 |
| ADI1 | 0.004591838 | 0.443967662 | 0.185 | 0.066 |
| PHLDA1 | 0.030290884 | 0.443610675 | 0.194 | 0.093 |
| IFITM2 | 0.002363584 | 0.442768783 | 0.293 | 0.159 |
| VAPA | 0.000775611 | 0.441126765 | 0.341 | 0.166 |
| PPP1CB | 0.000778641 | 0.440204579 | 0.392 | 0.205 |
| EIF4A3 | 0.008858375 | 0.44005455 | 0.268 | 0.132 |
| UBB | 0.000219235 | 0.434799619 | 0.675 | 0.53 |
| SELM | 0.001681685 | 0.434605069 | 0.242 | 0.099 |
| NFKBIZ | 4.06E−06 | 0.434420151 | 0.411 | 0.185 |
| UBR5 | 0.001580589 | 0.43256256 | 0.245 | 0.119 |
| PTPN14 | 0.013683922 | 0.432325155 | 0.197 | 0.086 |
| RAN | 0.004630671 | 0.43174956 | 0.258 | 0.132 |
| PPP2CA | 0.000580136 | 0.430118867 | 0.178 | 0.053 |
| CKS2 | 0.004674706 | 0.430092192 | 0.162 | 0.053 |
| ELF3 | 0.002186236 | 0.428997726 | 0.516 | 0.338 |
| EIF4A1 | 0.020341781 | 0.428722445 | 0.226 | 0.113 |
| CSTB | 0.000303017 | 0.42794926 | 0.354 | 0.172 |
| TSPYL2 | 0.00730692 | 0.427188478 | 0.188 | 0.073 |
| PLEC | 0.009444359 | 0.426830947 | 0.232 | 0.106 |
| UGDH | 0.00050297 | 0.426240027 | 0.28 | 0.126 |
| IBTK | 0.032659238 | 0.425092505 | 0.162 | 0.073 |
| TUBA1C | 0.000656294 | 0.424419126 | 0.162 | 0.04 |
| HLA-A | 0.000169504 | 0.421777538 | 0.592 | 0.377 |
| PHYHD1 | 0.002666132 | 0.420472535 | 0.159 | 0.046 |
| RNA28S5 | 5.36E−06 | 0.418447932 | 0.914 | 0.788 |
| UBA52 | 0.000254416 | 0.418312051 | 0.532 | 0.318 |
| RPL30 | 0.000211356 | 0.417781724 | 0.745 | 0.563 |
| FLOT1 | 0.016409315 | 0.416908668 | 0.156 | 0.073 |
| CHST9 | 0.029293937 | 0.414815916 | 0.328 | 0.212 |
| TMEM14B | 0.021464601 | 0.413962274 | 0.153 | 0.06 |
| EIF1 | 0.000363613 | 0.413691226 | 0.688 | 0.523 |
| PTHLH | 0.002855891 | 0.413037118 | 0.459 | 0.298 |
| STAT6 | 0.000396936 | 0.412588454 | 0.21 | 0.093 |
| LRRC8A | 0.004583975 | 0.4123547 | 0.162 | 0.066 |
| MTRNR2L2 | 0.000626941 | 0.411504077 | 0.975 | 0.94 |
| VPS37B | 0.001165157 | 0.411224801 | 0.162 | 0.046 |
| HN1 | 0.006192531 | 0.411106226 | 0.21 | 0.086 |
| ELK4 | 0.044190037 | 0.410158317 | 0.159 | 0.073 |
| NBN | 0.04900049 | 0.409527183 | 0.156 | 0.073 |
| MID1 | 0.013076353 | 0.408862424 | 0.217 | 0.099 |
| RAB2A | 0.004818383 | 0.406951798 | 0.194 | 0.073 |
| CCND2 | 5.71E−05 | 0.406925129 | 0.229 | 0.073 |
| PSMB1 | 0.001159013 | 0.406019831 | 0.369 | 0.192 |
| KDSR | 0.002735661 | 0.406007291 | 0.178 | 0.06 |
| SIK1 | 0.000508597 | 0.4056306 | 0.255 | 0.106 |
| KDM2A | 0.000420496 | 0.405040279 | 0.21 | 0.079 |
| CDC42SE2 | 0.021095569 | 0.404106299 | 0.201 | 0.093 |
| SLC35F5 | 0.041910868 | 0.402662582 | 0.162 | 0.086 |
| CLTA | 0.01086536 | 0.402610363 | 0.303 | 0.166 |
| SF3B14 | 0.003668233 | 0.400756674 | 0.201 | 0.079 |
| TUBA1B | 0.002446763 | 0.400738071 | 0.296 | 0.146 |
| C6orf48 | 0.001399514 | 0.399309199 | 0.197 | 0.073 |
| SRSF3 | 0.00032832 | 0.399210226 | 0.506 | 0.298 |
| RPS12 | 2.77E−05 | 0.399010555 | 0.85 | 0.735 |
| ARHGAP18 | 0.001227866 | 0.398301565 | 0.185 | 0.06 |
| C16orf72 | 0.009176432 | 0.396827664 | 0.153 | 0.073 |
| ALDOA | 6.09E−05 | 0.396684714 | 0.554 | 0.325 |
| MRFAP1 | 0.016828612 | 0.396117357 | 0.182 | 0.079 |
| LYPLAL1 | 0.006324582 | 0.395027079 | 0.153 | 0.079 |
| JUND | 1.66E−05 | 0.394251298 | 0.287 | 0.132 |
| TRAK1 | 0.002871218 | 0.393254739 | 0.271 | 0.139 |
| MINK1 | 0.001951555 | 0.393022109 | 0.162 | 0.053 |
| RPL11 | 8.82E−05 | 0.392196066 | 0.796 | 0.636 |
| HMGN2 | 0.00631407 | 0.391695832 | 0.258 | 0.146 |
| LNX2 | 0.029913211 | 0.390903964 | 0.153 | 0.073 |
| CFL1 | 0.001482675 | 0.390079412 | 0.436 | 0.252 |
| TAGLN2 | 0.012020383 | 0.389612853 | 0.338 | 0.199 |
| SPRY2 | 0.031569094 | 0.388554785 | 0.194 | 0.093 |
| HLA-C | 0.000210815 | 0.387942658 | 0.691 | 0.503 |
| SDC4 | 0.00681183 | 0.387312246 | 0.274 | 0.146 |
| EEF2 | 0.000114273 | 0.386615935 | 0.742 | 0.543 |
| PCBP1 | 0.004829255 | 0.386538513 | 0.226 | 0.099 |
| PER3 | 1.85E−05 | 0.384538589 | 0.217 | 0.066 |
| RPLP1 | 0.000369652 | 0.383587005 | 0.882 | 0.801 |
| RPL8 | 0.000343921 | 0.383510117 | 0.739 | 0.556 |
| REV1 | 0.018675471 | 0.383184662 | 0.213 | 0.106 |
| S100A2 | 0.000335115 | 0.38231514 | 0.761 | 0.583 |
| NDUFV1 | 0.0367067 | 0.38220848 | 0.166 | 0.086 |
| EIF3K | 0.038621305 | 0.38184711 | 0.277 | 0.166 |
| MYADM | 8.84E−06 | 0.380844934 | 0.207 | 0.053 |
| DDX3Y | 0.01385643 | 0.379923022 | 0.197 | 0.106 |
| KRT8 | 0.008104507 | 0.379318126 | 0.465 | 0.305 |
| SLPI | 4.72E−08 | 0.379127771 | 0.745 | 0.457 |
| CHD4 | 0.000197468 | 0.378206755 | 0.341 | 0.159 |
| SEMA5A | 0.00466938 | 0.377124817 | 0.366 | 0.205 |
| TOR1AIP2 | 0.000418682 | 0.377065678 | 0.204 | 0.066 |
| CHD2 | 0.000231251 | 0.376767472 | 0.369 | 0.192 |
| GSN | 0.002967165 | 0.37665576 | 0.28 | 0.139 |
| GDI2 | 0.016630854 | 0.376360367 | 0.223 | 0.139 |
| HLA-B | 0.000158333 | 0.375032206 | 0.806 | 0.649 |
| FLRT3 | 0.002124978 | 0.373891276 | 0.169 | 0.053 |
| PERP | 9.56E−05 | 0.37341827 | 0.701 | 0.563 |
| DNAJA1 | 0.000185325 | 0.3732145 | 0.532 | 0.318 |
| OBSCN | 0.005427731 | 0.370786524 | 0.175 | 0.066 |
| TNFRSF1A | 0.006552912 | 0.370298302 | 0.162 | 0.066 |
| PSMB3 | 0.000730895 | 0.36917421 | 0.217 | 0.079 |
| KLF6 | 0.004739395 | 0.368329928 | 0.443 | 0.272 |
| WDR26 | 0.039899467 | 0.367679349 | 0.182 | 0.106 |
| TFCP2L1 | 0.000383137 | 0.367147044 | 0.207 | 0.073 |
| ZCRB1 | 6.21E−05 | 0.366750698 | 0.226 | 0.073 |
| LITAF | 0.010429815 | 0.365805246 | 0.223 | 0.113 |
| SNRNP70 | 0.02724088 | 0.365011561 | 0.213 | 0.139 |
| EIF3I | 0.009525217 | 0.364777612 | 0.204 | 0.086 |
| SPINT1 | 0.007015894 | 0.363913363 | 0.162 | 0.06 |
| FLOT2 | 0.037639879 | 0.363759703 | 0.172 | 0.093 |
| SLC3A2 | 0.00650464 | 0.363121704 | 0.236 | 0.106 |
| UGT2A2 | 0.010964688 | 0.362885696 | 0.347 | 0.205 |
| ID2 | 1.83E−07 | 0.359787026 | 0.334 | 0.126 |
| TMEM59 | 0.034810861 | 0.358653777 | 0.449 | 0.318 |
| HMGA1 | 0.000363494 | 0.357963567 | 0.188 | 0.073 |
| CTNND1 | 0.005371885 | 0.357858261 | 0.312 | 0.166 |
| FKBP9 | 0.004874376 | 0.357525554 | 0.252 | 0.119 |
| TUBB4B | 0.02292893 | 0.357423183 | 0.29 | 0.166 |
| POLR2G | 0.000176318 | 0.35685514 | 0.159 | 0.046 |
| SLC9A3R1 | 0.008788222 | 0.356835349 | 0.239 | 0.113 |
| SNHG5 | 0.000352824 | 0.356818932 | 0.666 | 0.464 |
| HEBP2 | 0.003659759 | 0.356707657 | 0.258 | 0.119 |
| RNF181 | 0.011074256 | 0.35547859 | 0.162 | 0.06 |
| BCAM | 0.019725583 | 0.355411657 | 0.417 | 0.272 |
| SRI | 0.030123716 | 0.355169329 | 0.252 | 0.139 |
| ARHGDIA | 0.000662561 | 0.35332113 | 0.153 | 0.04 |
| HERPUD1 | 0.01939782 | 0.352792856 | 0.258 | 0.139 |
| KLF10 | 0.010256649 | 0.352736658 | 0.303 | 0.166 |
| MAT2A | 0.002837238 | 0.351909225 | 0.239 | 0.126 |
| LEPROT | 0.009928744 | 0.349394107 | 0.245 | 0.119 |
| SPCS1 | 0.008131936 | 0.348515113 | 0.213 | 0.099 |
| SORL1 | 0.044357017 | 0.347693088 | 0.252 | 0.139 |
| CCNL1 | 0.010515337 | 0.347429661 | 0.471 | 0.325 |
| CYB5R3 | 0.011793546 | 0.346917892 | 0.169 | 0.066 |
| USP22 | 0.044444486 | 0.346188784 | 0.226 | 0.119 |
| MKNK2 | 0.01364999 | 0.345597673 | 0.207 | 0.099 |
| SEC14L1 | 0.026134762 | 0.345195521 | 0.191 | 0.093 |
| SEC61B | 0.002250132 | 0.344248794 | 0.229 | 0.132 |
| BTF3 | 0.00151727 | 0.344217429 | 0.631 | 0.457 |
| FZD6 | 0.049117049 | 0.342809492 | 0.175 | 0.099 |
| DAZAP2 | 0.010132508 | 0.341671834 | 0.309 | 0.172 |
| SFN | 0.009625081 | 0.341563337 | 0.389 | 0.238 |
| MGST1 | 0.007874737 | 0.341065527 | 0.239 | 0.113 |
| LAMB3 | 0.004817925 | 0.340968703 | 0.43 | 0.278 |
| TNFRSF12A | 0.017784414 | 0.340847522 | 0.175 | 0.073 |
| OAT | 0.012508357 | 0.340795271 | 0.455 | 0.298 |
| CD99 | 0.00270562 | 0.340367978 | 0.354 | 0.192 |
| CAV2 | 0.014710869 | 0.339586633 | 0.182 | 0.079 |
| FLNA | 0.014232357 | 0.338960198 | 0.398 | 0.265 |
| STXBP3 | 0.00071318 | 0.33888245 | 0.159 | 0.053 |
| RIPK4 | 0.000898422 | 0.338355835 | 0.188 | 0.066 |
| SRSF7 | 0.015372265 | 0.338344474 | 0.433 | 0.298 |
| EIF3D | 0.000800003 | 0.338174263 | 0.252 | 0.119 |
| TNPO1 | 0.046381433 | 0.33691204 | 0.191 | 0.093 |
| MYL12B | 0.003582154 | 0.335775549 | 0.57 | 0.391 |
| GCLC | 0.00213876 | 0.335542117 | 0.331 | 0.172 |
| METTL7A | 0.025414113 | 0.33517075 | 0.258 | 0.146 |
| RPL28 | 0.004954045 | 0.335132679 | 0.589 | 0.43 |
| F2R | 0.000160706 | 0.333591528 | 0.277 | 0.119 |
| RNA18S5 | 0.040327428 | 0.333450508 | 0.465 | 0.358 |
| TMEM261 | 0.000573271 | 0.333322287 | 0.188 | 0.066 |
| SOD1 | 0.00671605 | 0.333101416 | 0.443 | 0.278 |
| YBX3 | 0.02270734 | 0.332817588 | 0.376 | 0.252 |
| C11orf31 | 0.015522085 | 0.331559057 | 0.287 | 0.159 |
| MYL6 | 0.000338989 | 0.331188888 | 0.691 | 0.483 |
| ELOVL5 | 0.004940269 | 0.331134803 | 0.207 | 0.086 |
| ATOX1 | 0.041072038 | 0.331126877 | 0.156 | 0.066 |
| RPLP2 | 0.002602355 | 0.331048756 | 0.729 | 0.636 |
| FBXL5 | 0.000557922 | 0.330688152 | 0.22 | 0.086 |
| MAP3K13 | 0.010960718 | 0.330283961 | 0.162 | 0.06 |
| MDK | 0.005644061 | 0.32921461 | 0.312 | 0.166 |
| TOB1 | 0.006390272 | 0.328758858 | 0.459 | 0.291 |
| DLL1 | 0.024204228 | 0.326382672 | 0.226 | 0.113 |
| HSP90AB1 | 0.004151741 | 0.326205321 | 0.662 | 0.497 |
| SNCA | 5.85E−05 | 0.325909783 | 0.271 | 0.106 |
| LAMA3 | 0.034107806 | 0.32561197 | 0.328 | 0.212 |
| CSNK1A1 | 0.013490597 | 0.325099088 | 0.309 | 0.172 |
| ATP1B3 | 0.021819856 | 0.323756195 | 0.293 | 0.172 |
| RPS2 | 0.009462836 | 0.323470741 | 0.436 | 0.278 |
| EIF4H | 0.043807214 | 0.322602938 | 0.162 | 0.073 |
| UBE2D3 | 0.005894211 | 0.321473576 | 0.341 | 0.212 |
| CEBPB | 0.019285201 | 0.32116501 | 0.191 | 0.086 |
| TGFBR2 | 0.036128047 | 0.320565427 | 0.175 | 0.079 |
| RPS3 | 0.000829843 | 0.319916882 | 0.704 | 0.536 |
| GADD45B | 0.013564678 | 0.319547302 | 0.468 | 0.331 |
| GAS5 | 0.000269657 | 0.319360972 | 0.576 | 0.384 |
| ZNHIT1 | 0.007298701 | 0.318608563 | 0.185 | 0.073 |
| DDR1 | 0.037737924 | 0.318589836 | 0.277 | 0.159 |
| JAG2 | 0.018392062 | 0.31805894 | 0.169 | 0.079 |
| WNT4 | 0.016778908 | 0.317648304 | 0.166 | 0.066 |
| NDFIP1 | 0.021677707 | 0.316779181 | 0.252 | 0.132 |
| APH1A | 0.001346928 | 0.316715983 | 0.156 | 0.046 |
| OAZ1 | 0.00048763 | 0.316322048 | 0.51 | 0.311 |
| HDAC7 | 0.010515806 | 0.315501088 | 0.169 | 0.066 |
| ATP1A1 | 0.005468062 | 0.313692561 | 0.624 | 0.457 |
| FAU | 0.008135288 | 0.313268737 | 0.599 | 0.43 |
| PPL | 0.003226472 | 0.312748376 | 0.315 | 0.166 |
| RAB11A | 0.039337469 | 0.312404015 | 0.21 | 0.106 |
| NCOA4 | 0.000673708 | 0.311939472 | 0.414 | 0.278 |
| ATP5O | 0.008560769 | 0.311681986 | 0.274 | 0.146 |
| SF1 | 0.010970349 | 0.310510232 | 0.268 | 0.146 |
| UQCRQ | 0.049781022 | 0.309744446 | 0.366 | 0.238 |
| NONO | 0.049666169 | 0.309479121 | 0.28 | 0.166 |
| FBXL3 | 0.040736826 | 0.309400512 | 0.156 | 0.066 |
| ID3 | 1.82E−05 | 0.309236239 | 0.194 | 0.06 |
| DHCR24 | 0.044826413 | 0.308009009 | 0.35 | 0.232 |
| IER5 | 0.002650253 | 0.30759493 | 0.153 | 0.053 |
| OPTN | 0.038987266 | 0.307342843 | 0.166 | 0.086 |
| TMSB10 | 0.003571227 | 0.306445208 | 0.621 | 0.45 |
| PPP1CA | 0.018841018 | 0.306296552 | 0.207 | 0.099 |
| ARPC5 | 0.00216332 | 0.305903027 | 0.274 | 0.132 |
| RAB1A | 0.006517651 | 0.304880995 | 0.248 | 0.119 |
| TSKU | 0.022971495 | 0.30454631 | 0.197 | 0.093 |
| RPL19 | 0.004794513 | 0.30451265 | 0.764 | 0.629 |
| BTG1 | 0.007872218 | 0.304382132 | 0.385 | 0.232 |
| NACA | 0.008322238 | 0.30430417 | 0.586 | 0.417 |
| RAI14 | 0.007086435 | 0.303921159 | 0.274 | 0.159 |
| CAPN1 | 0.00871982 | 0.30365468 | 0.252 | 0.126 |
| 10-Sep | 0.047077266 | 0.303045791 | 0.156 | 0.079 |
| CDKN2AIP | 0.01970989 | 0.302770255 | 0.162 | 0.066 |
| PHB2 | 0.005952706 | 0.302642983 | 0.178 | 0.073 |
| DDOST | 0.008890061 | 0.302405365 | 0.226 | 0.106 |
| S100A16 | 0.002012545 | 0.301930297 | 0.178 | 0.066 |
| BLCAP | 0.023776392 | 0.301677827 | 0.217 | 0.119 |
| CSDE1 | 0.002025165 | 0.301067427 | 0.478 | 0.298 |
| EDF1 | 0.005665162 | 0.29837772 | 0.261 | 0.132 |
| CDK5RAP2 | 0.026994838 | 0.296960282 | 0.191 | 0.093 |
| PRKAR1A | 0.001621206 | 0.296558959 | 0.395 | 0.252 |
| CNN2 | 0.009116869 | 0.296412985 | 0.277 | 0.146 |
| FGFR3 | 0.037375556 | 0.295872334 | 0.373 | 0.245 |
| ALDH3A2 | 0.024534092 | 0.294793135 | 0.494 | 0.344 |
| TMX4 | 0.042110383 | 0.293953613 | 0.162 | 0.079 |
| RPL7A | 0.002915318 | 0.293865334 | 0.611 | 0.444 |
| RPS27 | 0.030602229 | 0.293089838 | 0.615 | 0.497 |
| C14orf166 | 0.019719366 | 0.292470131 | 0.162 | 0.066 |
| RPL18 | 0.006000873 | 0.292405691 | 0.637 | 0.497 |
| C7orf55-LUC7L2 | 0.013001169 | 0.292048029 | 0.182 | 0.079 |
| GNG5 | 0.021622017 | 0.291871539 | 0.185 | 0.086 |
| GHITM | 0.011112217 | 0.291097539 | 0.223 | 0.113 |
| IVNS1ABP | 0.024662244 | 0.290920068 | 0.519 | 0.397 |
| H3F3B | 0.001464735 | 0.289665443 | 0.726 | 0.556 |
| RBM3 | 0.001919282 | 0.289632737 | 0.484 | 0.311 |
| RPL27 | 0.01433765 | 0.289009828 | 0.707 | 0.57 |
| SMIM15 | 0.010675758 | 0.288316657 | 0.182 | 0.079 |
| ARFGAP3 | 0.021299087 | 0.287969118 | 0.159 | 0.066 |
| PPP4R1 | 0.036472327 | 0.287724169 | 0.268 | 0.152 |
| NDUFB4 | 0.046344907 | 0.287181693 | 0.213 | 0.113 |
| SSBP1 | 0.010931699 | 0.287018606 | 0.242 | 0.119 |
| RPS21 | 0.000418368 | 0.286954311 | 0.748 | 0.576 |
| DDB1 | 0.000537702 | 0.286922339 | 0.197 | 0.073 |
| ALAS1 | 0.003087924 | 0.286833236 | 0.175 | 0.066 |
| SERINC3 | 0.020440709 | 0.286331884 | 0.229 | 0.119 |
| TMBIM6 | 0.002718351 | 0.285597432 | 0.561 | 0.377 |
| AQP5 | 0.002630865 | 0.28413308 | 0.299 | 0.159 |
| RPS5 | 0.001691685 | 0.281673662 | 0.685 | 0.497 |
| EIF3F | 0.003696093 | 0.280692595 | 0.236 | 0.113 |
| 7-Mar | 0.020857039 | 0.279055211 | 0.182 | 0.086 |
| SDF4 | 0.001154683 | 0.277682009 | 0.201 | 0.079 |
| RPL15 | 0.0023104 | 0.275660103 | 0.768 | 0.596 |
| CDC42SE1 | 0.028840804 | 0.273649681 | 0.191 | 0.093 |
| LGALS8 | 0.009837622 | 0.272471617 | 0.315 | 0.179 |
| PAK1 | 0.008111101 | 0.270735811 | 0.239 | 0.119 |
| NAMPT | 0.040342304 | 0.270597944 | 0.188 | 0.106 |
| EGFR | 0.019140455 | 0.26992163 | 0.519 | 0.364 |
| NDUFS1 | 0.002000443 | 0.269484517 | 0.162 | 0.06 |
| GPR155 | 0.023384147 | 0.268257572 | 0.217 | 0.113 |
| RAD23B | 0.036167339 | 0.268160434 | 0.185 | 0.099 |
| CD9 | 0.007226882 | 0.267890309 | 0.768 | 0.616 |
| LIMK2 | 0.031839981 | 0.267448187 | 0.22 | 0.119 |
| DLK2 | 0.006932147 | 0.267245079 | 0.229 | 0.113 |
| CBR1 | 0.005674981 | 0.26633294 | 0.414 | 0.265 |
| PPP2R5C | 0.008955953 | 0.265650013 | 0.156 | 0.06 |
| PRRC2C | 0.005965786 | 0.265147005 | 0.379 | 0.232 |
| RPS11 | 0.001603767 | 0.264258196 | 0.758 | 0.583 |
| SH3GLB1 | 0.018275945 | 0.263356531 | 0.236 | 0.126 |
| HNRNPA0 | 0.000285039 | 0.261747689 | 0.22 | 0.086 |
| NUMA1 | 0.001534269 | 0.26162255 | 0.299 | 0.152 |
| FGFR2 | 0.043429354 | 0.260765085 | 0.185 | 0.099 |
| GABARAPL2 | 0.022294029 | 0.260411686 | 0.182 | 0.093 |
| ETV3 | 0.004559235 | 0.259863685 | 0.159 | 0.06 |
| H3F3C | 0.00477045 | 0.258343631 | 0.207 | 0.093 |
| SSH1 | 0.001859114 | 0.257528951 | 0.194 | 0.079 |
| ATF4 | 0.026697193 | 0.257528777 | 0.35 | 0.225 |
| SH3BGRL | 0.038085192 | 0.257391464 | 0.166 | 0.079 |
| ABHD2 | 4.91E−05 | 0.25723398 | 0.258 | 0.106 |
| ZFYVE21 | 0.002733328 | 0.255829892 | 0.239 | 0.113 |
| RPS24 | 0.000504033 | 0.25546935 | 0.777 | 0.603 |
| SYNCRIP | 0.028163769 | 0.255134622 | 0.306 | 0.185 |
| DDX17 | 0.035556594 | 0.252824615 | 0.567 | 0.424 |
| RPS8 | 0.000548611 | 0.251276768 | 0.825 | 0.728 |
| AFG3L2 | 5.80E−05 | 0.250323291 | 0.178 | 0.053 |
| RPL26 | 4.14E−05 | −0.2528291 | 0.576 | 0.55 |
| HNRNPA3 | 0.046550477 | −0.253086169 | 0.169 | 0.212 |
| VPS26A | 0.032204028 | −0.253583758 | 0.166 | 0.166 |
| CHD8 | 0.042455805 | −0.254305873 | 0.146 | 0.152 |
| RPS19 | 0.018237473 | −0.258851256 | 0.777 | 0.801 |
| NCL | 0.040841744 | −0.258866603 | 0.481 | 0.51 |
| PDCD10 | 0.033199805 | −0.261414118 | 0.131 | 0.166 |
| DST | 0.02621095 | −0.26649736 | 0.736 | 0.755 |
| POMP | 0.014610517 | −0.26874517 | 0.213 | 0.232 |
| APP | 0.001193003 | −0.269107091 | 0.599 | 0.609 |
| CLU | 0.019922885 | −0.270329913 | 0.252 | 0.252 |
| SRP9 | 0.038619801 | −0.271971009 | 0.258 | 0.285 |
| SKP1 | 0.041659996 | −0.27847759 | 0.43 | 0.464 |
| RPL41 | 0.003388116 | −0.280463693 | 0.592 | 0.623 |
| RPL39 | 0.00456969 | −0.286698059 | 0.404 | 0.444 |
| UQCRB | 0.007621458 | −0.290573231 | 0.248 | 0.258 |
| MTUS1 | 0.01552845 | −0.29240054 | 0.226 | 0.245 |
| IFT81 | 0.006373458 | −0.292776389 | 0.166 | 0.139 |
| USP8 | 0.019362279 | −0.299474962 | 0.175 | 0.219 |
| RPL6 | 0.000597671 | −0.300767326 | 0.478 | 0.49 |
| RNF19A | 0.000499791 | −0.30129322 | 0.191 | 0.192 |
| ADAM28 | 0.005709789 | −0.302348492 | 0.551 | 0.689 |
| LSM3 | 0.030930017 | −0.30283949 | 0.156 | 0.159 |
| ASPH | 0.009052108 | −0.30644683 | 0.338 | 0.358 |
| GOLGB1 | 0.006981883 | −0.308255348 | 0.446 | 0.57 |
| HNRNPH1 | 0.030354455 | −0.314986612 | 0.478 | 0.583 |
| CP | 4.85E−05 | −0.320845865 | 0.532 | 0.517 |
| TPM3 | 0.011067566 | −0.323745058 | 0.255 | 0.272 |
| EEF1D | 0.000154765 | −0.326224033 | 0.392 | 0.417 |
| PDIA4 | 0.004545675 | −0.326834767 | 0.21 | 0.219 |
| PTMA | 9.11E−05 | −0.32702689 | 0.58 | 0.603 |
| CMYA5 | 0.001352917 | −0.327925015 | 0.166 | 0.265 |
| PGAP1 | 0.014866228 | −0.328265427 | 0.105 | 0.166 |
| MPHOSPH6 | 0.029876698 | −0.332819808 | 0.131 | 0.159 |
| MTRNR2L8 | 3.87E−06 | −0.344358273 | 0.917 | 0.907 |
| MALAT1 | 7.37E−12 | −0.34859182 | 0.981 | 1 |
| VMO1 | 0.002718446 | −0.350243789 | 0.344 | 0.358 |
| ITGAV | 0.041243607 | −0.355430423 | 0.207 | 0.252 |
| PDIA3 | 0.002178949 | −0.358661897 | 0.389 | 0.45 |
| TLK1 | 0.013078367 | −0.363824862 | 0.121 | 0.166 |
| RPL21 | 0.002653706 | −0.369797565 | 0.127 | 0.166 |
| MTRNR2L10 | 0.016637296 | −0.376712035 | 0.29 | 0.384 |
| CXADR | 0.0015789 | −0.378996042 | 0.271 | 0.305 |
| SPCS2 | 0.012133472 | −0.381367978 | 0.111 | 0.159 |
| HSPA5 | 0.000447951 | −0.38614652 | 0.462 | 0.497 |
| POSTN | 6.79E−05 | −0.38716639 | 0.736 | 0.722 |
| LARS | 0.021031074 | −0.389330179 | 0.188 | 0.232 |
| MORF4L1 | 2.82E−07 | −0.392331425 | 0.318 | 0.278 |
| ATP5I | 0.001240081 | −0.392741609 | 0.303 | 0.358 |
| TNC | 0.000404365 | −0.39413028 | 0.417 | 0.457 |
| AKAP9 | 0.00748685 | −0.398779415 | 0.443 | 0.57 |
| TMSB4X | 3.74E−05 | −0.408300346 | 0.503 | 0.53 |
| BPTF | 0.013669503 | −0.410633466 | 0.182 | 0.318 |
| SESN3 | 0.006722407 | −0.411216772 | 0.172 | 0.219 |
| SYNE2 | 0.000504426 | −0.414455132 | 0.545 | 0.649 |
| TTC3 | 0.033882359 | −0.417913411 | 0.169 | 0.258 |
| C1R | 0.021483148 | −0.421304224 | 0.127 | 0.205 |
| RPL10 | 1.90E−08 | −0.448167858 | 0.468 | 0.444 |
| MT-ND6 | 0.001426585 | −0.468317973 | 0.073 | 0.192 |
| SERPINB4 | 0.011279756 | −0.468919249 | 0.162 | 0.232 |
| PHPT1 | 0.004157427 | −0.471042808 | 0.137 | 0.219 |
| RPL34 | 2.19E−07 | −0.473354078 | 0.79 | 0.834 |
| IGFBP2 | 0.001087901 | −0.475035087 | 0.137 | 0.159 |
| MTRNR2L1 | 1.62E−07 | −0.488347191 | 0.834 | 0.901 |
| SLC38A1 | 0.014653401 | −0.498429694 | 0.086 | 0.152 |
| SMC6 | 0.002838309 | −0.500689228 | 0.124 | 0.212 |
| ENAH | 0.005191755 | −0.502109355 | 0.185 | 0.238 |
| SON | 9.11E−05 | −0.514226605 | 0.424 | 0.543 |
| HLA-DRA | 0.000658043 | −0.527661506 | 0.134 | 0.258 |
| YWHAE | 3.39E−06 | −0.533576077 | 0.331 | 0.43 |
| HSP90B1 | 6.29E−07 | −0.56742065 | 0.487 | 0.609 |
| MTRNR2L7 | 2.10E−05 | −0.57618243 | 0.245 | 0.384 |
| MTRNR2L5 | 4.30E−07 | −0.627684303 | 0.363 | 0.517 |
| MTRNR2L11 | 0.000206164 | −0.646720716 | 0.194 | 0.325 |
| MT-ND2 | 8.16E−13 | −0.739058556 | 0.58 | 0.748 |
| MTRNR2L3 | 1.51E−14 | −0.782372654 | 0.573 | 0.795 |
| CTC-338M12.5 | 1.33E−06 | −0.797667915 | 0.035 | 0.192 |
| MTRNR2L13 | 3.15E−08 | −0.836715132 | 0.121 | 0.291 |
| MMP10 | 8.23E−11 | −0.963579237 | 0.452 | 0.503 |
| MT-RNR1 | 1.43E−37 | −0.984019822 | 0.99 | 1 |
| IGHA1 | 2.75E−13 | −0.987869127 | 0.003 | 0.179 |
| IGJ | 8.26E−11 | −1.007079308 | 0.006 | 0.159 |
| MT-ATP8 | 2.40E−10 | −1.038077569 | 0.124 | 0.384 |
| MT-ND1 | 4.18E−19 | −1.04750878 | 0.455 | 0.768 |
| MT-ND5 | 7.27E−22 | −1.07152441 | 0.545 | 0.854 |
| MT-RNR2 | 1.09E−57 | −1.142226582 | 1 | 1 |
| MT-CO2 | 1.53E−31 | −1.234456602 | 0.752 | 0.921 |
| MT-CYB | 2.19E−36 | −1.24835121 | 0.576 | 0.921 |
| MT-ATP6 | 5.53E−28 | −1.2817996 | 0.49 | 0.801 |
| MT-ND4L | 5.65E−24 | −1.312288502 | 0.325 | 0.735 |
| MT-CO3 | 3.49E−50 | −1.561604516 | 0.71 | 0.967 |
| IGKC | 1.49E−21 | −1.578112603 | 0.003 | 0.291 |
| MT-ND4 | 2.12E−50 | −1.680248879 | 0.557 | 0.947 |
| MT-CO1 | 2.18E−60 | −1.695353716 | 0.697 | 0.974 |

It was next sought to understand how changes in the basal epithelium propagated through to secretory cells. To address this, changes in secretory cells recovered from scrapings of both the inferior turbinate and accessible polyp tissue pre- and post-dupilumab treatment was looked in parallel (FIG. 19e). After identifying differentiating/secretory cells related to those found in (FIG. 2e) within inferior turbinate and polyp scrapings (FIG. 19e,f; Methods), candidate pathways of interest were tested, and a specific decrease in Wnt-target genes (Wnt pathway gene set, CD44), a significant decrease in IL-4/IL-13 induced genes (IL-4/IL-13 gene set, ALOX15), and a concomitant increase in IFNα induced genes (IFNα gene set, IF127) in polyps were observed (FIG. 19g). The healthy and diseased secretory cell gene sets were utilized (FIG. 3e) to identify a core gene set conserved with treatment (WFDC2, S100A6, PIGR). Pairwise differential expression tests on samples grouped by anatomical location and treatment identified sets of genes expressed in healthy inferior turbinate restored in polyp secretory cells (S100A9, LCN2, MUC1) and genes restored in both inferior turbinate and polyp secretory cells (ANKRD36C, MUC4, RARRES1; FIG. 19h). Furthermore, a significant decrease in the expression of non-polyp secretory genes (TPT1, SCGB1A1, DUSP1), polyp secretory cell genes (CST1, TFF3, EGLN3), and the transcription factors ATF3, KLF5, and FOSB was observed (FIG. 19h). This suggests that even though CRS samples from the ethmoid sinus have unique secretory cell signatures observed in non-polyp and polyp tissues (FIG. 3e), cytokine blockade leads to expression of genes associated with healthy inferior turbinate secretory cells, even in ethmoid sinus derived polyp tissue (FIG. 19h). Further investigation will be required to formally test whether this represents the natural potential of these cells in a non-inflammatory environment (FIG. 6a). The exploration of the shifts induced by IL-4Ra blockade within the basal and secretory cells of an individual requires further examination. However, it highlights the potential of single-cell approaches to deconvolve tissues, allowing for in vivo validation of in vitro mechanisms in humans relating to shifts in specific cell states and differentiation potential of accessible barrier tissues. Additionally, healthy genes that were not restored by treatment can be targeted as residual genes independent of IL-4/IL-13 antagonists (e.g., HLA-B, SAT1, C15orf48, S100A8, RNA28S5, CEACAM6, CEACAM5, KRT7, ALDH3A1, S100A4, S100P, LYPD2 and PSCA).

One fundamental goal of understanding the cellular and molecular pathways activated in T2I is to provide mechanisms that may explain the dramatically increased incidence of chronic allergic inflammatory disease, particularly beyond conventional immune mechanisms¹⁴⁹. While a signature of immune cell infiltration dominates bulk transcriptomic analyses of polyps relative to non-polyp tissue in CRS (FIG. 8), by utilizing scRNA-seq in combination with a well-defined patient cohort across the severity spectrum of a human chronic allergic inflammatory disease, this study provides descriptive, mechanistic and functional insights into an enigmatic cell state, and the consequences it has for the differentiation of a productive barrier. The data may help to explain why nasal polyposis is associated with infections by specific microorganisms⁸, and how a monoclonal antibody targeting the shared IL-4/IL-13 receptor can reduce nasal polyp burden¹⁵⁰ Specifically, striking differences are shown in the antimicrobial effectors produced by secretory cells relative to healthy tissue, a loss of glandular cell heterogeneity (including those producing MUC5B⁹²), and that IL-4/IL-13 strongly induce a transcriptional program already at the level of basal progenitor cells¹⁵¹. These results suggest that therapeutic modulation of basal cell differentiation during chronic inflammation could help to partially restore homeostatic balance. Taken together with recent work in the murine intestinal tract and skin^{21,152-154}, this work provides human evidence for the emerging paradigm of stem cell dysfunction altering the set point of a barrier tissue. Specifically, substantial overlap is highlighted amongst putative driving transcription factors (ATF3, AP-1, TP63, and KLF5) with those identified in a murine model of psoriasiform skin inflammation¹⁵⁴. This demonstrates that the principle of inflammatory memory¹²⁸ underlying barrier tissue adaptation is a generalizable phenomenon observed in two distinct anatomical locations, inflammatory modules, and species. These findings are built on by culturing basal cells ex vivo and identifying the indelible mark of IL-4 and IL-13 as a baseline induction of the Wnt pathway driven by CTNNB1 expression. While the immune response in T2I may represent an attempt to reset appropriate biodiversity within a tissue (FIG. 4b, FIG. 14e), based on these results, it is proposed that basal cells can form “memories” of chronic exposure to an inflammatory T2I environment, shifting the entire specialized cellular ecosystem away from productive differentiation, and propagating disease. Future work will be needed to determine the relative contributions of memory stored in distinct compartments—by cells classically viewed to drive allergic inflammation, such as T and B cells, and within the epithelium itself¹⁵⁵—to develop the most effective mechanisms by which to erase them.

### Methods

Study Participants and Design for Single-Cell Study from Ethmoid Sinus Tissue

Subjects between the ages of 18 and 75 years were recruited from the Brigham and Women's Hospital (Boston, Mass.) Allergy and Immunology clinic and Otolaryngology clinic between May 2014 and August 2017. The Institutional Review Board approved the study, and all subjects provided written consent. Ethmoid sinus tissue was collected at the time of elective endoscopic sinus surgery from patients with physician-diagnosed CRS with and without nasal polyps based on established guidelines¹⁵⁶. Patients with polyps include both aspirin-tolerant chronic rhinosinusitis with nasal polyps (CRSwNP) and individuals with aspirin-exacerbated respiratory disease (AERD). Patients were suspected of having AERD if they had asthma, nasal polyposis, and a history of respiratory reaction on ingestion of a COX 1 inhibitor, with confirmation via a graded oral challenge to aspirin. Subjects with cystic fibrosis and unilateral polyps were excluded from the study.

Tissue segments (one per patient) for bulk tissue RNA-seq was immediately placed in RNAlater (Qiagen) for RNA extraction, and for patient samples loaded on Seq-Well and for flow-sorting to ATAC-seq, tissue was received in-hand, placed in RPMI (Corning) with 10% FBS (ThermoFisher 10082-147) and immediately on ice for transport.

NB: Originally, a healthy control subject with no history of CRS or nasal polyposis who was undergoing sinus surgery for concha bullosa was enrolled. However, this subject upon pathology evaluation was noted to have mild eosinophilia, a chart review revealed a history of allergic rhinitis and asthma, and their diagnosis was updated to CRSsNP clinically by the surgeon upon follow-up visits so their status was updated accordingly in this study. Additionally, non-polyp patient 6 was sampled twice (denoted as 6A and 6B), representing distinct cells that were captured on two different Seq-Well arrays. As such, they should not be viewed as a technical replicate.

Collection of Inferior Turbinate and Nasal Polyp Samples through Nasal Scraping

Nasal samples were collected from the inferior turbinate of healthy control subjects and from the inferior turbinate and accessible polyp tissue in subjects with CRSwNP using the Rhino-Pro® Curette, a sterile, disposable, mucosal collection device, as described^{57,158}. One sample was taken from the right and left mid-inferior portion of the inferior turbinate using a gentle scraping motion. In two subjects with CRSwNP, with accessible nasal polyp tissue, the polyp tissue was sampled using the Rhino-Pro® Curette under direct visualization. The nasal scrapings sampled were placed directly in RPMI with 10% FBS and immediately on ice for transport.

One subject with CRSwNP and co-morbid severe atopic dermatitis was started on dupilumab, a human monoclonal antibody that binds to the IL-4Ra subunit approved for severe atopic dermatitis¹⁵⁹. The inferior turbinate and nasal polyp tissue was sampled with the Rhino-Pro® Curette pre- and post-treatment with 3 doses of dupilumab, and through endoscopic sinus surgery as noted above.

### Tissue Digestion

Single-cell suspensions from collected surgical specimens were obtained using a modified version of a previously published protocol⁴¹, described below in detail. Each specimen was received directly in hand and processed directly with an average time from patient to loading onto the SeqWell platform of 3 total hours, and never exceeding 4 hours. Surgical specimens were collected into 30 mL of ice cold RPMI (Corning). Specimens were finely minced between two scalpel blades and incubated for 15 minutes at 37° C. in a rotisserie rack with end-over-end rotation in 25 mL digestion buffer supplemented with 600 U/mL collagenase IV (Worthington) and 20 ug/mL DNAse 1 (Roche) in RPMI with 10% fetal bovine serum. After 15 minutes, samples were triturated five times using a syringe with a 16G needle and returned to the rotisserie rack for another 15 minutes. At the conclusion of the second digest period, samples were triturated an additional five times using a syringe with a 16G needle, at which point the digest process was stopped via the addition of EDTA to 20 mM. Nasal scrapings were only dissociated with one 15 minute dissociation via collagenase and omission of the 16G needle trituration, instead replaced with P1000 pipette trituration, as typically cell yields were <20,000 total cells. Processing downstream remained identical. Samples were typically fully dissociated at this step and were filtered through a 70 uM cell strainer and spun down at 500G for 10 minutes followed a rinse with ice-cold PBS (ThermoFisher 10010023, Ca/Mg free) to 30 mL total volume. RBCs were lysed using ACK buffer (ThermoFisher A1049201) for 3 minutes on ice to remove red blood cells, even if no RBC contamination was visibly seen in order to maintain consistency across patient groups. Cells were then washed with sterile PBS and spun down at 500G for 5 minutes, resuspended in complete RPMI medium with 2% FCS (RPMI1640 [ThermoFisher 61870-127], 100 U/ml penicillin [ThermoFisher 15140-122], 100 ug/mL streptomycin [ThermoFisher 15140-122], 10 mM HEPES [ThermoFisher 15630-080], 2% FCS [ThermoFisher 10082-147], 50 ug/mL gentamicin [ThermoFisher 15750-060]), and counted to adjust concentration to 100,000 cells/mL for loading onto SeqWell arrays.

### Flow Cytometry, Cell Sorting, and Analysis

Single-cell suspensions in FACS Buffer (HBSS [ThermoFisher 14170161, Ca/MgFree] supplemented with 2% FCS) were pre-incubated with Fc Block before staining for surface antigens. The following antibodies were used to identify basal cells via flow cytometry: FITC anti-human THY1 (Biolegend, clone 5E10), Brilliant Violet 421 anti-human CD45 (Biolegend, clone HI130), Brilliant Violet 650 anti-human EPCAM (Biolegend, clone 9C4), APC/Cy7 anti-human ITGA6 (Biolegend, clone GoH3), PE/Cy7 anti-human NGFR (Biolegend, clone ME20.4), APC anti-human PDPN (Biolegend, clone NC-08). Cells were stained for 30 minutes on ice in FACS buffer and then washed for immediate sorting. Cells were sorted on a BD FACSAria Fusion cell sorter using BD FACSDiva software. Up to 10,000 Basal cells were sorted into 100 μL BAM banker (Wako chemicals) for ATAC sequencing and cooled to −80° C. using a “Mr. Frosty” freezing container (Thermo scientific). Samples were stored at −80° C. until thawed for omni-ATAC-seq. 1,000 Basal cells for bulk RNAseq were sorted directly into 5 μL TCL buffer (Qiagen). FlowJo v10 by TreeStar was used to generate plots.

### Histologic Analyses

Biopsies were fixed in 4% paraformaldehyde, embedded in paraffin, and 6 m sections were prepared and stained with hematoxylin and eosin for quantification of glandular areas. Photomicrographs encompassing the entire area of each biopsy were taken. Total and glandular areas were measured with Image J software and expressed as glandular area as a percentage of total area. For p63 immunofluoresence, sections were quenched for 10 minutes in 1 mg/mL sodium borohydride in PBS. For antigen retrieval, slides were placed in a Coplin jar with preheated citrate tarket retrieval buffer (DAKO) at 95° C. and transferred to a steamer for 60 minutes. Slides were cooled for 20 minutes at room temperature and then transferred to distilled water followed by PBS. Samples were blocked with serum free protein block (DAKO) containing 5% normal donkey serum for 60 minutes. Samples were incubated overnight at 4C with purified anti-TP63 antibody (Biolegend, clone W15093A). After three washes in PBS-T, samples were incubated with 1:500 AlexaFluor 647-conjugated donkey anti-mouse IgG (Jackson immunoresearch, catalog #715-605-150) and 1:10,000 Hoescht nuclear dye. Quantification of p63+ cells was performed in a blinded fashion and involved counting of p63+ nuclei relative to background staining with an isotype control primary antibody. As the epithelium can vary in area, Applicants normalized our quantification of total positive nuclei per 1000 μm² of epithelium as measured in ImageJ and report the final value as p63+ cells/1000 μm² of epithelial area.

### Single-Cell RNA-Sequencing

Once a single-cell suspension was obtained from freshly resected sinus tissue, or scrapings from inferior turbinate, the Seq-Well platform was utilized for massively parallel scRNA-seq to capture transcriptomes of single cells on barcoded mRNA capture beads. Full methods on implementation of this platform are available in Gierahn et al.¹⁵. Briefly, 20,000 cells were loaded onto one array preloaded with barcoded mRNA capture beads (ChemGenes). The loaded arrays containing cells and beads were then sealed using a polycarbonate membrane with a pore size of 0.01 μm, which allows for exchange of buffers but retains biological molecules confined within each nanowell. Subsequent exchange of buffers allows for cell lysis, transcript hybridization, and bead recovery before performing reverse transcription en masse. Following reverse transcription using Maxima H Minus Reverse Transcriptase (ThermoFisher EP0753) and an Exonuclease I treatment (NewEngland Biolabs M0293L) to remove excess primers, PCR amplification was carried out using KAPA HiFi PCR Mastermix (Kapa Biosystems KK2602) with 2,000 beads per 50 uL reaction volume. Libraries were then pooled in sets of six (totaling 12,000 beads) and purified using Agencourt AMPure XP beads (Beckman Coulter, A63881) by a 0.6× SPRI followed by a 0.7×SPRI and quantified using Qubit hsDNA Assay (Thermo Fisher Q32854). Quality of WTA product was assessed using the Agilent hsD5000 Screen Tape System (Agilent Genomics) with an expected peak >1000 bp tailing off to beyond 5000 bp, and a small/non-existent primer peak, indicating a successful preparation. Libraries were constructed using the Nextera XT DNA tagmentation method (Illumina FC-131-1096) on a total of 600 pg of pooled cDNA library from 12,000 recovered beads using index primers with format as in Gierahn et al¹⁵. Tagmented and amplified sequences were purified at a 0.6×SPRI ratio yielding library sizes with an average distribution of 650-750 base pairs in length as determined using the Agilent hsD 1000 Screen Tape System (Agilent Genomics). Two arrays were sequenced per sequencing run with an Illumina 75 Cycle NextSeq500/550v2 kit (Illumina FC-404-2005) at a final concentration of 2.8 pM. The read structure was paired end with Read 1 starting from a custom read 1 primer⁵ containing 20 bases with a 12 bp cell barcode and 8 bp unique molecular identifier (UMI) and Read 2 containing 50 bases of transcript information.

### Single-Cell RNA-Sequencing Computational Pipelines and Analysis

Read alignment was performed as in Macosko et al¹⁶⁰. Briefly, for each NextSeq sequencing run, raw sequencing data was converted to demultiplexed FASTQ files using bcl2fastq2 based on Nextera N700 indices corresponding to individual samples/arrays. Reads were then aligned to hg19 genome using the Galaxy portal maintained by the Broad Institute for Drop-Seq alignment using standard settings. Individual reads were tagged according to the 12-bp barcode sequenced and the 8-bp UMI contained in Read 1 of each fragment. Following alignment, reads were binned onto 12-bp cell barcodes and collapsed by their 8-bp UMI. Digital gene expression matrices (e.g. cells-by-genes tables) for each sample were obtained from quality filtered and mapped reads, with an automatically determined threshold for cell count. UMI-collapsed data was utilized as input into Seurat²⁹ (github.com/satijalab/seurat) for further analysis. Before incorporating a sample into the merged dataset, the cells-by-genes matrix of each was individually inspected as a Seurat object.

For analysis of all sequenced surgical ethmoid sinus resection samples, UMI matrices were merged across all genes detected in any condition and generated a matrix retaining all cells with at least 500 UMI detected (n=19,196 cells and 31,032 genes). This table was then utilized to setup the Seurat object in which any cell with at least 300 unique genes was retained and any gene expressed in at least 5 cells was retained (Supplementary Information: an R Script is included from this point to set up Seurat object and walk reader through dimensionality reduction and basic data visualization). The object was initiated with log-normalization, from a UMI+1 count matrix, scaling, and centering set to True. The total number of cells passing these filters captured across all patients was 18,624 cells with 22,575 genes, averaging 1,503 cells per sample with a range between 789 cells and 3,109 cells. Before performing dimensionality reduction, data was subset to include cells with less than 12,000 UMI, and a list of 1,627 most variable genes was generated by including genes with an average normalized and scaled expression value greater than 0.13 and with a dispersion (variance/mean) greater than 0.28. Principal component analysis was then performed over the list of variable genes. For both clustering and t-stochastic neighbor embedding (tSNE), the first 12 principal components were utilized, as upon visual inspection of genes contained within, each contributed to a non-redundant cell type and this reflected the inflection point of the elbow plot. FindClusters (which utilizes a shared nearest neighbor (SNN) modularity optimization based clustering algorithm) was used with a resolution of 1.2 and tSNE set to Fast with the Barnes-hut implementation to identify 21 clusters across the 12 input samples.

For analysis of all sequenced surgical ethmoid sinus resection samples, UMI matrices across all genes detected in any condition were merged and a matrix retaining all cells with at least 500 UMI detected (n=19,196 cells and 31,032 genes) was generated. This table was then utilized to setup the Seurat object in which any cell with at least 300 unique genes was retained and any gene expressed in at least 5 cells was retained (Supplementary Information: an R Script is included from this point to set up Seurat object and walk reader through dimensionality reduction and basic data visualization). The object was initiated with log-normalization, from a UMI+1 count matrix, scaling, and centering set to True. The total number of cells passing these filters captured across all patients was 18,624 cells with 22,575 genes, averaging 1,503 cells per sample with a range between 789 cells and 3,109 cells. Before performing dimensionality reduction, data was subset to include cells with less than 12,000 UMI, and a list of 1,627 most variable genes was generated by including genes with an average normalized and scaled expression value greater than 0.13 and with a dispersion (variance/mean) greater than 0.28. Principal component analysis was then performed over the list of variable genes. For both clustering and t-stochastic neighbor embedding (tSNE), the first 12 principal components were utilized, as upon visual inspection of genes contained within, each contributed to a non-redundant cell type and this reflected the inflection point of the elbow plot. FindClusters (which utilizes a shared nearest neighbor (SNN) modularity optimization based clustering algorithm) was used with a resolution of 1.2 and tSNE set to Fast with the Barnes-hut implementation to identify 21 clusters across the 12 input samples.

For analysis of all sequenced inferior turbinate scraping samples, the object was initiated with log-normalization, from a UMI+1 count matrix, scaling, and centering set to True. The total number of cells passing these filters captured across all patients was 18,704 cells with 24,842 genes, averaging 2,078 cells per sample with a range between 65 cells and 5,625 cells (NB: The 65 cell sample was a very mucus-laden polyp inferior turbinate sample, perhaps explaining the low cell yield, but clustered well within the three other samples each containing 253, 599, and 1,381 cells). Before performing dimensionality reduction, data was subset to include cells with less than 10,000 UMI, and a list of 1,499 most variable genes was generated by including genes with an average normalized and scaled expression value greater than 0.22 and with a dispersion (variance/mean) greater than 0.26. Principal component analysis was then performed over the list of variable genes. For both clustering and t-stochastic neighbor embedding (tSNE), the first 16 principal components were utilized, as upon visual inspection of genes contained within, each contributed to a non-redundant cell type and this reflected the inflection point of the elbow plot. FindClusters (which utilizes a shared nearest neighbor (SNN) modularity optimization based clustering algorithm) was used with a resolution of 1 and tSNE set to Fast with the Barnes-hut implementation to identify 18 clusters across the 9 input samples.

For analysis of all sequenced ALI cultures, the object was initiated with log-normalization, from a UMI+1 count matrix, scaling, and centering set to True. The total number of cells passing these filters captured across all patients was 16,173 cells with 27,396 genes, averaging 2,448 cells per sample with a range between 1,980 cells and 3,009 cells. Before performing dimensionality reduction, data was subset to include cells with less than 25,000 UMI, and a list of 1,670 most variable genes was generated by including genes with an average normalized and scaled expression value greater than 0.35 and with a dispersion (variance/mean) greater than 0.35. Principal component analysis was then performed over the list of variable genes. For both clustering and t-stochastic neighbor embedding (tSNE), the first 16 principal components were utilized, as upon visual inspection of genes contained within, each contributed to a non-redundant cell type and this reflected the inflection point of the elbow plot. FindClusters (which utilizes a shared nearest neighbor (SNN) modularity optimization based clustering algorithm) was used with a resolution of 0.6 and tSNE set to Fast with the Barnes-hut implementation to identify 11 clusters across the 4 input samples.

Cell Type Identification and within Cell Type Analysis

To identify genes which defined each cluster, a ROC test implemented in Seurat was performed with a threshold set to an area under the curve of 0.65. Top marker genes with high specificity were used to classify cell subsets into cell types (FIG. 1a,b,c) based on existing biological knowledge. Three clusters were considered doublets (n=588 cells) based on co-expression of markers indicative of distinct cell types at ˜1/2 the expression level detected in the parent cell cluster (e.g. T cell and myeloid cell) and removed from further analyses yielding a matrix with 18,036 cells used in all subsequent steps. Closely related clusters were merged to cell types based on biological curation and analysis of hierarchical cluster trees yielding ten total cell types (FIG. 1a,b,c). A much smaller number of eosinophils were identified than expected in our single-cell data. However, the attempts to isolate RNA from eosinophils or eosinophil-rich tissue (such as eosinophilic esophagitis) indicates that their RNA is quite labile. Specifically, if tissue is not placed immediately into RNA-later within 10 minutes, eosinophil associated transcripts can not be reliably detected, and preliminary SeqWell experiments on patients with active eosinophilic esophagitis also yielded low capture of eosinophils (data not shown). However, flow cytometrically from 0.5% to 5% of total cells fitting eosinophil profiles from polyps were recovered, and single-cell studies were focused on granulocytes at the expense of the full ecosystem are possible and the topic of future work (data not shown). A distinct cluster of ILCs was also not found as they are around 0.01 to 0.1% of CD45 cells across the CRS spectrum per existing literature⁴³ and extrapolating to the number of CD45 cells captured, between 0.8 and 8 ILCs would have been detected. To investigate further granularity present within cell types, such as T cells, myeloid cells, fibroblasts, endothelial cells, and epithelial cells, these cells from the Seurat object were subset and re-ran dimensionality reduction and clustering (FIGS. 9, 10 and 13). The process used for clustering and subset identification was adapted for each cell type to optimize the parameters of variable genes, principal components, and resolution of clusters desired.

### Differential Expression and Fractional Contribution of Gene Set to Transcriptome,

To identify differentially expressed genes within cell types across non-polyp and polyp disease states, the ‘bimod’ setting was used in FindMarkers implemented in Seurat based on a likelihood ratio test designed for single-cell differential expression incorporating both a discrete and continuous component¹⁶¹. To determine the expression contribution to a cell's transcriptome of a particular gene list, the total log-normalized expression values for genes within a “list of interest” were summed and divided by the total amount of log-normalized transcripts detected in that cell, giving the proportion of a cell's transcriptome dedicated to producing those genes. For comparison of Wnt and Notch signaling, the expression contribution metric was z-scored and the value of Notch from Wnt was subtracted yielding a metric centered on zero if both scores are equivalent, or weighted in the positive direction if enriched in Wnt.

Simpson's Index of Diversity, and Fibroblast Gene Correlation with Basal Cell Frequency

To measure the “richness” of the epithelial ecosystem, Simpson's Index of Diversity (D) was employed, which were presented as (1-D), and ranges between 0 and 1, with greater values indicating larger sample diversity¹¹³. This measure takes into account the total number of members of a cell type, the number of cell types, and the total number of cells present. (1-D) was calculated for each sample. To determine genes correlated in specific cell types (e.g. fibroblasts) with the frequency of basal cells present in a cellular ecosystem, the average log-normalized single-cell count data was correlated for each gene to the rank of samples determined by increasing frequency of basal cells in each ecosystem (8.2% to 19.1% for non-polyp and 27.9% to 70.1% for polyp samples, FIG. 14b).

### Tissue and Sorted Basal Cell RNA-Sequencing

Population RNA-seq was performed using a derivative of the Smart-Seq2 protocol for single cells¹⁶². In brief, tissue was collected directly into RNAlater (Qiagen) in the surgical suite and stored at −80 until RNA isolation. RNA was isolated from 30 patients using phenol/chloroform extraction and normalized to 5 ng as the input amount for a 2.2×SPRI ratio cleanup using Agencourt RNAClean XP beads (Beckman Coulter, A63987). RNA-seq from sorted basal cells was done as a bulk population using Smart-Seq2 chemistry starting with a 2.2×SPRI ratio cleanup. After oligo-dT priming, Maxima H Minus Reverse Transcriptase (ThermoFisher EP0753) was utilized to synthesize cDNA with an elongation step at 52° C. before PCR amplification (15 cycles for tissue, 18 cycles for sorted basal cells) using KAPA HiFi PCR Mastermix (Kapa Biosystems KK2602). Sequencing libraries were prepared using the Nextera XT DNA tagmentation kit (Illumina FC-131-1096) with 250 pg input for each sample. Libraries were pooled post-Nextera and cleaned using Agencourt AMPure SPRI beads with successive 0.7× and 0.8× ratio SPRIs and sequenced with an Illumina 75 Cycle NextSeq500/550v2 kit (Illumina FC-404-2005) with loading density at 2.2 pM, with paired end 35 cycle read structure. Tissue samples were sequenced at an average read depth of 7.98 million reads per sample and 3 samples not meeting quality thresholds were excluded from further analyses yielding 27 total useable samples. Sorted basal cell samples were sequenced at an average read depth of 21.15 million reads per sample and all samples met quality thresholds regarding genomic and transcriptomic alignment.

### Tissue and Sorted Basal Cell RNA-Sequencing Data Analysis

Tissue and sorted basal cell samples were aligned to the Hg19 genome and transcriptome using STAR¹⁶³ and RSEM¹⁶⁴. 3 samples were excluded for low transcriptome alignment (<25%), so 27 samples were retained for further analyses. Differential expression analysis was conducted using DESeq2 package for R¹⁶⁵. Genes regarded as significantly differentially expressed were determined based on an adjusted p-value using a Benjamini-Hochberg adjusted p-value to correct for multiple comparisons with a false discovery rate <0.05. Ingenuity Pathway Analysis (IPA, Qiagen) was performed through an instance available through the Broad Institute on the top 1000 genes (all adjusted p<0.05) differentially expressed from our DESeq2 analysis, taking into account corresponding log-fold change for each gene. The tissue RNA-seq matrix was also subset based on genes found in Tables 1-15, which, from the single-cell marker discovery, were specific for basal, differentiating/secretory, glandular, or ciliated cells. PCA and KNN clustering implemented in R were then run over these genes in order to identify the greatest vectors of variance across samples within the epithelial cell compartment.

For re-analysis of published data, two publically-available RNA-seq data sets were used: one profiling normal human olfactory mucosa and the other assessing differences in gene expression between healthy, non-eosinophilic nasal polyps and eosinophilic nasal polyps^{8,114,115}NB: analysis is done per sample and as such no comparisons across the data sets or samples are made.

### Diffusion Pseudotime Mapping for Differentiation Analysis

Diffusion pseudotime¹²² was calculated using the scanpy python package ‘dpt’ function on log normalized data separately for clusters 8, 1 and 4 (predominantly non-polyp) and 12, 2, and 0 (predominantly polyp) together by. A random root cell was chosen from cluster 8, as this was the basal cell cluster representative of the non-polyp (e.g. less aberrant) state, and iterations were also run with random root cells chosen from the entire set of clusters and cluster 8 was assigned as the cluster most enriched at the beginning of the diffusion map, regardless. Plots were created with the seaborn, matplotlib, and pandas packages. Pearson correlations were then calculated for all genes in all cells tested, or for all genes in non-polyp cells and all genes in polyp cells, relative to pseudotime. Differential correlation testing was performed using the cocor package to identify significance for the difference between correlation coefficients using Fisher's 1925 z-statistic, account for number of cells.

### Epigenetic Profiling of Basal Cells Using Omni-ATAC-Seq

Accessible chromatin profiling using the Omni-ATAC-Seq protocol as described in Corces et al¹²⁴ was performed on basal cells stored in 100 μL BAMBanker freezing media from 12 patients (n=4 non-polyp and n=8 polyp). Cells (ranging from 1,000 to 10,000) were thawed quickly in a 37° C. rock bath and 900 μL of ice-cold PBS supplemented with Roche complete-Mini Protease inhibitor was added immediately. Cells were split into two 1.5 mL Eppendorf DNA lo-bind tubes to serve as technical replicates. Cells were centrifuged at 500 g for 5 minutes at 4° C., washed once in PBS with protease inhibitor, centrifuged at 500 g for 5 minutes at 4° C. and supernatant was removed completely using two separate pipetting steps with extreme caution taken to avoid resuspension (e.g. smooth and consistent aspiration). The transposition reaction consisted of 20 μL total volume of the following mixture (10 μL 2×TD Buffer, 1 or 0.5 μL TDEnzyme, 0.1 μL of 2% digitonin, 0.2 μL of 10% Tween 20, 0.2 μL of 10% NP40, 6.6 μL of 1×PBS and 2.3 μL of nuclease free water). Replicates were performed with two distinct concentrations of TDE since, when dealing with minute clinical samples, flow sorting can sometimes give variable cell numbers, and the ratio of TDE to cells is critical in determining the frequency with which cuts are made in the genome. It was optimized in pilot experiments that for basal cell inputs in the range of 500 to 10,000 cells, the aforementioned two ratios gave expected patterns of nucleosome banding in gels (data not shown). Two reactions were performed and then later, during in silico analysis, peaks were pooled together for downstream analysis. The cells were resuspended into the transposition mixture and incubated at 37° C. for 30 minutes in an Eppendorf Thermomixer with agitation at 300 rpm. Transposed DNA was purified using a Qiagen MinElute Reaction Cleanup Kit with elution in 15 uL. Libraries were constructed from 10 μL of DNA using a 50 μL total reaction volume of NEB HF 2×PCR Master Mix with custom Nextera N700 and N500 index primers to barcode samples (also used in Smart-Seq2 protocol). 14 cycles of PCR amplification were performed and SPRI purified at 1.8× ratio. Based on the molarity of each library, the number of subsequent PCR cycles was adjusted to either 3, 4 or 5 more for each sample. A 0.25× reverse SPRI was then performed to remove larger fragments followed by a 1.7×SPRI to purify libraries for sequencing. Libraries were sequenced on an Illumina NextSeq with paired end 38 cycle read structure at a loading density of 1.95 pM.

### ATAC-Seq Data Analysis

Reads were aligned using bowtie2 using the following flags: “−S −p 1 −X 2000—chunkmbs 1000” then bams were created using samtools view with the following flags: ‘samtools view −bS −F 4 −”. Duplicates were removed with picard. Forward reads were shifted 4 bp and negative reads were shifted 5 bp using a custom python script and the pysam package as is recommended for ATAC-seq data. Samples for each patient were merged using samtools merge and all patients were downsampled to 3 million reads using custom python scripts and ‘samtools view’ with the ‘−b’ and ‘−s’ flags. MACS2 ‘callpeak’ command was used to call peaks on each sample with flags “‘−f BAMPE’-q 0.001--nomodel--shift-100--extsize 200-B -broad’. Peaks from all samples were merged into one peakfile with bedtools and counts of reads per peak for each sample was generated with bedtools multicov. DESeq2 was run with the design ˜polyp, testing for significant differences between polyp and non-polyp samples on this peak matrix and differential peaks with Benjamini-Hochberg adjusted p-value less than 0.01 with ‘greater’ or ‘less’ null hypotheses were used in downstream analysis. Homer2 was run for known motif finding on differential peaks with the set of all peaks as background¹⁶⁶. To determine a false discovery rate, Homer2 was run on sets of random peaks chosen with replacement from the set of all peaks.

### Epithelial Cell Culture

Tissues were digested as described above from either non-polyp or polyp surgical resections from the ethmoid sinus. 1,000,000 digested cells were added to a 25 cm² tissue culture flask (Corning) pre-coated with 0.03 mg/mL collagen solution (StemCell Technologies) and cultured in PneumaCult Ex media (Stemcell Technologies, 5008). Media was changed every second day until cells reached confluence. Cells were subsequently frozen in 70% basal media with 20% FBS and 10% DMSO.

### Air-Liquid Interface Cultures

For air-liquid interface cultures, 100,000 cultured epithelial cells per well were added to 0.4 um pore 24-well polyester membrane inserts (Corning) pre-coated with 0.03 mg/mL collagen solution (StemCell Technologies) with Pneumacult Ex media (Stemcell Technologies, 5008) on both sides of the membrane. After 24 hours, apical media was changed to remove dead cells. After 72 hours, apical media was removed completely and basal media was changed to Pneumacult ALI (Stemcell Technologies, 05001) supplemented with 5 mL 100× penicillin-streptomycin (Fisher), 1 mL 500× gentamicin/amphotericin B (ThermoFisher), 1 mL 0.2% heparin sodium salt in PBS (Stemcell Technologies) and 2.5 mL 200× hydrocortisone stock solution (Stemcell Technologies) and 0, 0.1, 1 or 10 ng/mL IL-13 (Biolegend). Basal media was changed every 2-3 days for 21 days, after which membranes were removed and cells dissociated with Stempro Accutase Cell Dissociateion Reagent (Gibco) for Seq-Well or flow cytometry.

### Basal Cell Stimulation

Basal cells from non-polyp or polyp surgical resections from ethmoid sinus were placed into epithelial cell culture (e.g. “lateral expansion” in the absence of differentiation, see above) were passaged, and 10,000 cells seeded at passage 5 (e.g. 5 weeks ex vivo) and cultured at confluence in 96 well flat-bottom collagen-coated tissue culture plates (Corning, 3799) for 48 hours in Pneumacult Ex serum-free media (Stemcell Technologies, 5008). Cytokines were added for 12 hours overnight at increasing doses (0, 0.1, 1, 10 ng/mL) of IL-4 (Biolegend 766205), IL-13 (Biolegend 571104), and (0.1 ng/mL) IL-4+IL-13 in combination (n=32 samples non-polyp and n=32 samples polyp basal cells over all conditions, each condition run as a biological duplicate, and a technical duplicate therein) before lysis using RLT+BME. Bulk RNA-seq was performed as described for sorted basal cells starting from lysates. Basal cell stimulation samples were sequenced at an average read depth of 3 million reads per sample and all samples met quality thresholds regarding genomic and transcriptomic alignment.

### Statistical Analyses

Number of samples included in analyses are listed throughout figure legends and all represent distinct biological samples. The same surgeon performed surgeries on all individuals and was independent of study design. The same allergist/immunologist performed nasal scrapings on all samples and was independent of study design. Quantification of histological sections was performed in a blinded fashion. No samples or cells meeting quality thresholds were excluded from analyses. Statistical analyses were performed using GraphPad Prism v7.0a, Seurat 1.4.0.1 implemented in RStudio, DESeq2 1.10.1 package implemented in RStudio, and Ingenuity Pathway Analysis run through the Broad Institute, and macs2, DESeq2 and Homer2 for omni-ATAC-Seq. All violin plots, which were elected to use due to zero-inflation in single-cell data, contain at minimum 498 individual data points in any one cluster, and have points suppressed for ease of legibility. Some violin plots with less than 498 cells have individual data points displayed and corresponding statistical metrics are available in accompanying figure legend. Violins are generated through default code implemented in Seurat for generation. For scores in single-cell data, effect sizes are reported in addition to statistical significance as an additional metric for the magnitude of the effect observed. The calculation was performed as Cohen's d where: effect size d=(Mean₁−Mean₂)/(S.D. pooled). Unpaired 2-tail t-tests for direct comparisons and t-test with Holm-Sidak correction, Bonferroni correction, or Benjamini-Hochberg for multiple comparisons, depending on software package used, where appropriate. Mann-Whitney U-test for quantification of histological data due to non-gaussian distribution. Pearson correlation thresholds were determined as significant through determination of asymptotic p-values through use of rcorr function in Hmisc, but exact corrected p-values by Holm-Sidak method for multiple comparisons are calculated for those highlighted in text using RcmdrMisc package. Comparison of Pearson correlation coefficients in pseudotime analyses was done using Fisher's 1925 z-statistic accounting for the number of cells.

### REFERENCES

1 Belkaid, Y. & Artis, D. Immunity at the barriers. Eur J Immunol 43, 3096-3097, doi:10.1002/eji.201344133 (2013).
2 Schleimer, R. P. & Berdnikovs, S. Etiology of epithelial barrier dysfunction in patients with type 2 inflammatory diseases. J Allergy Clin Immunol 139, 1752-1761, doi:10.1016/j.jaci.2017.04.010 (2017).
3 Hogan, B. L. et al. Repair and regeneration of the respiratory system: complexity, plasticity, and mechanisms of lung stem cell function. Cell Stem Cell 15, 123-138, doi:10.1016/j.stem.2014.07.012 (2014).
4 Whitsett, J. A. & Alenghat, T. Respiratory epithelial cells orchestrate pulmonary innate immunity. Nat Immunol 16, 27-35, doi: 10.1038/ni.3045 (2015).
Iwasaki, A., Foxman, E. F. & Molony, R. D. Early local immune defences in the respiratory tract. Nat Rev Immunol 17, 7-20, doi: 10.1038/nri.2016.117 (2017).
6 Hammad, H. & Lambrecht, B. N. Barrier Epithelial Cells and the Control of Type 2 Immunity. Immunity 43, 29-40, doi: 10. 1016/j. immuni.2015.07.007 (2015).
7 Holtzman, M. J., Byers, D. E., Alexander-Brett, J. & Wang, X. The role of airway epithelial cells and innate immune cells in chronic respiratory disease. Nat Rev Immunol 14, 686-698, doi:10.1038/nri3739 (2014).
8 Schleimer, R. P. Immunopathogenesis of Chronic Rhinosinusitis and Nasal Polyposis. Annu Rev Pathol 12, 331-357, doi:10.1146/annurev-pathol-052016-100401 (2017).
9 Al-Rawi, M. M., Edelstein, D. R. & Erlandson, R. A. Changes in nasal epithelium in patients with severe chronic sinusitis: a clinicopathologic and electron microscopic study. Laryngoscope 108, 1816-1823 (1998).
Li, C. W. et al. Role of p63/p73 in epithelial remodeling and their response to steroid treatment in nasal polyposis. J Allergy Clin Immunol 127, 765-772 e761-762, doi:10.1016/j.jaci.2010.12.011 (2011).
11 Zhao, L. et al. Increase of poorly proliferated p63(+)/Ki67(+) basal cells forming multiple layers in the aberrant remodeled epithelium in nasal polyps. Allergy 72, 975-984, doi:10.1111/all.13074 (2017).
12 Hansel, F. K. Clinical and Histopathological Studies of the Nose and Sinuses in Allergy Journal of Allergy 1, 43-70 (1929).
13 Teixeira, V. H. et al. Stochastic homeostasis in human airway epithelium is achieved by neutral competition of basal cell progenitors. Elife 2, e00966, doi:10.7554/eLife.00966 (2013).
14 Rock, J. R. et al. Basal cells as stem cells of the mouse trachea and human airway epithelium. Proc Natl Acad Sci USA 106, 12771-12775, doi:10.1073/pnas.0906850106 (2009).
Gierahn, T. M. et al. Seq-Well: portable, low-cost RNA sequencing of single cells at high throughput. Nat Methods 14, 395-398, doi:10.1038/nmeth.4179 (2017).
16 Cheng, L. E. & Locksley, R. M. Allergic inflammation-innately homeostatic. Cold Spring Harb Perspect Biol 7, a016352, doi: 10.1 101/cshperspect.a016352 (2014).
17 Palm, N. W., Rosenstein, R. K. & Medzhitov, R. Allergic host defences. Nature 484, 465-472, doi: 10.1038/nature 11047 (2012).
18 Iwasaki, A. & Medzhitov, R. Control of adaptive immunity by the innate immune system. Nat Immunol 16, 343-353, doi:10.1038/ni.3123 (2015).
19 Wills-Karp, M. et al. Interleukin-13: central mediator of allergic asthma. Science 282, 2258-2261 (1998).
20 Kotas, M. E. & Medzhitov, R. Homeostasis, inflammation, and disease susceptibility. Cell 160, 816-827, doi:10.1016/j.cell.2015.02.010 (2015).
21 Karin, M. & Clevers, H. Reparative inflammation takes charge of tissue regeneration. Nature 529, 307-315, doi:10.1038/naturel7039 (2016).
22 Gieseck, R. L., 3rd, Wilson, M. S. & Wynn, T. A. Type 2 immunity in tissue repair and fibrosis. Nat Rev Immunol, doi: 10.103 8/nri.2017.90 (2017).
23 Soyka, M. B. et al. Defective epithelial barrier in chronic rhinosinusitis: the regulation of tight junctions by IFN-gamma and IL-4. J Allergy Clin Immunol 130, 1087-1096 el010, doi:10.1016/j.jaci.2012.05.052 (2012).
24 Bernstein, J. M., Gorfien, J., Noble, B. & Yankaskas, J. R. Nasal polyposis: immunohistochemistry and bioelectrical findings (a hypothesis for the development of nasal polyps). J Allergy Clin Immunol 99, 165-175 (1997).
25 Collins, M. H. et al. Newly developed and validated eosinophilic esophagitis histology scoring system and evidence that it outperforms peak eosinophil count for disease diagnosis and monitoring. Dis Esophagus 30, 1-8, doi: 10.1111/dote. 12470 (2017).
26 Hong, K. U., Reynolds, S. D., Watkins, S., Fuchs, E. & Stripp, B. R. Basal cells are a multipotent progenitor capable of renewing the bronchial epithelium. Am J Pathol 164, 577-588, doi:10.1016/S0002-9440(10)63147-1 (2004).
27 Guttman-Yassky, E. et al. Broad defects in epidermal cornification in atopic dermatitis identified through genomic analysis. J Allergy Clin Immunol 124, 1235-1244 e1258, doi:10.1016/j.jaci.2009.09.031 (2009).
28 Heymann, P. W. et al. Rhinovirus infection results in stronger and more persistent genomic dysregulation: Evidence for altered innate immune response in asthmatics at baseline, early in infection, and during convalescence. PLoS One 12, e0178096, doi:10.1371/journal.pone.0178096 (2017).
29 Satija, R., Farrell, J. A., Gennert, D., Schier, A. F. & Regev, A. Spatial reconstruction of single-cell gene expression data. Nat Biotechnol 33, 495-502, doi:10.1038/nbt. 3192 (2015).
Purkis, P. E. et al. Antibody markers of basal cells in complex epithelia. J Cell Sci 97 (Pt 1), 39-50 (1990).
31 Yu, X., Ng, C. P., Habacher, H. & Roy, S. Foxj1 transcription factors are master regulators of the motile ciliogenic program. Nat Genet 40, 1445-1453, doi:10.1038/ng.263 (2008).
32 Fukami, M., Stierna, P., Veress, B. & Carlsoo, B. Lysozyme and lactoferrin in human maxillary sinus mucosa during chronic sinusitis. An immunohistochemical study. Eur Arch Otorhinolaryngol 250, 133-139 (1993).
33 Raphael, G. D. et al. Pathophysiology of rhinitis. Lactoferrin and lysozyme in nasal secretions. J Clin Invest 84, 1528-1535, doi:10.1172/JCI114329 (1989).
34 Thiriot, A. et al. Differential DARC/ACKR1 expression distinguishes venular from non-venular endothelial cells in murine tissues. BMC Biol 15, 45, doi:10.1186/s12915-017-0381-7 (2017).
Zheng, B., Zhang, Z., Black, C. M., de Crombrugghe, B. & Denton, C. P. Ligand-dependent genetic recombination in fibroblasts: a potentially powerful technique for investigating gene function in fibrosis. Am J Pathol 160, 1609-1617, doi:10.1016/S0002-9440(10)61108-X (2002).
36 Mason, D. Y. et al. The IgM-associated protein mb-1 as a marker of normal and neoplastic B cells. J Immunol 147, 2474-2482 (1991).
37 Guilliams, M. et al. Unsupervised High-Dimensional Analysis Aligns Dendritic Cells across Tissues and Species. Immunity 45, 669-684, doi:10.1016/j.immuni.2016.08.015 (2016).
38 Banchereau, J. & Steinman, R. M. Dendritic cells and the control of immunity. Nature 392, 245-252, doi:10.1038/32588 (1998).
39 Hedrick, S. M., Cohen, D. I., Nielsen, E. A. & Davis, M. M. Isolation of cDNA clones encoding T cell-specific membrane-associated proteins. Nature 308, 149-153 (1984).
40 Yanagi, Y. et al. A human T cell-specific cDNA clone encodes a protein having extensive homology to immunoglobulin chains. Nature 308, 145-149 (1984).
41 Dwyer, D. F., Barrett, N. A., Austen, K. F. & Immunological Genome Project, C. Expression profiling of constitutive mast cells reveals a unique identity within the immune system. Nat Immunol 17, 878-887, doi:10.1038/ni.3445 (2016).
42 Steinert, E. M. et al. Quantifying Memory CD8 T Cells Reveals Regionalization of Immunosurveillance. Cell 161, 737-749, doi:10.1016/j.cell.2015.03.031 (2015).
43 Poposki, J. A. et al. Group 2 innate lymphoid cells are elevated and activated in chronic rhinosinusitis with nasal polyps. Immun Inflamm Dis 5, 233-243, doi:10.1002/iid3.161 (2017).
44 Weller, P. F. & Spencer, L. A. Functions of tissue-resident eosinophils. Nat Rev Immunol, doi: 10.1038/nri.2017.95 (2017).
45 Villani, A. C. et al. Single-cell RNA-seq reveals new types of human blood dendritic cells, monocytes, and progenitors. Science 356, doi:10.1126/science.aah4573 (2017).
46 Gordon, S. & Martinez, F. O. Alternative activation of macrophages: mechanism and functions. Immunity 32, 593-604, doi: 10. 1016/j.immuni.2010.05.007 (2010).
47 Okabe, Y. & Medzhitov, R. Tissue biology perspective on macrophages. Nat Immunol 17, 9-17, doi:10.1038/ni.3320 (2015).
48 Zepp, J. A. et al. Distinct Mesenchymal Lineages and Niches Promote Epithelial Self-Renewal and Myofibrogenesis in the Lung. Cell 170, 1134-1148 el110, doi:10.1016/j.cell.2017.07.034 (2017).
49 Ohlund, D., Elyada, E. & Tuveson, D. Fibroblast heterogeneity in the cancer wound. J Exp Med 211, 1503-1523, doi:10.1084/jem.20140692 (2014).
50 von Andrian, U. H. & Mackay, C. R. T-cell function and migration. Two sides of the same coin. NEngl J Med 343, 1020-1034, doi:10.1056/NEJM200010053431407 (2000).
51 Swamy, M., Jamora, C., Havran, W. & Hayday, A. Epithelial decision makers: in search of the ‘epimmunome’. Nat Immunol 11, 656-665, doi: 10.1038/ni. 1905 (2010).
52 Maravillas-Montero, J. L. et al. Cutting edge: GPR35/CXCR8 is the receptor of the mucosal chemokine CXCL17. J Immunol 194, 29-33, doi: 10.4049/j immunol. 1401704 (2015).
53 Lazarus, N. H. et al. A common mucosal chemokine (mucosae-associated epithelial chemokine/CCL28) selectively attracts IgA plasmablasts. J Immunol 170, 3799-3805 (2003).
54 Fanning, L. B. & Boyce, J. A. Lipid mediators and allergic diseases. Ann Allergy Asthma Immunol 111, 155-162, doi:10.1016/j.anai.2013.06.031 (2013).
55 Miyoshi, H. et al. Prostaglandin E2 promotes intestinal repair through an adaptive cellular response of the epithelium. EMBO J 36, 5-24, doi:10. 15252/embj.201694660 (2017).
56 Zhao, J. et al. Preferential Generation of 15-HETE-PE Induced by IL-13 Regulates Goblet Cell Differentiation in Human Airway Epithelial Cells. Am J Respir Cell Mol Biol 57, 692-701, doi: 10.1165/rcmb.2017-00310OC (2017).
57 Wenzel, S. E. et al. PEBP1 Wardens Ferroptosis by Enabling Lipoxygenase Generation of Lipid Death Signals. Cell 171, 628-641 e626, doi:10.1016/j.cell.2017.09.044 (2017).
58 von Moltke, J., Ji, M., Liang, H. E. & Locksley, R. M. Tuft-cell-derived IL-25 regulates an intestinal ILC2-epithelial response circuit. Nature 529, 221-225, doi:10.1038/nature16161 (2016).
59 Schmitz, J. et al. IL-33, an interleukin-1-like cytokine that signals via the IL-1 receptor-related protein ST2 and induces T helper type 2-associated cytokines. Immunity 23, 479-490, doi:10.1016/j.immuni.2005.09.015 (2005).
60 Barlow, J. L. et al. IL-33 is more potent than IL-25 in provoking IL-13-producing nuocytes (type 2 innate lymphoid cells) and airway contraction. J Allergy Clin Immunol 132, 933-941, doi:10.1016/j.jaci.2013.05.012 (2013).
61 Moussion, C., Ortega, N. & Girard, J. P. The IL-1-like cytokine IL-33 is constitutively expressed in the nucleus of endothelial cells and epithelial cells in vivo: a novel ‘alarmin’? PLoS One 3, e3331, doi:10.1371/journal.pone.0003331 (2008).
62 Allakhverdi, Z. et al. Thymic stromal lymphopoietin is released by human epithelial cells in response to microbes, trauma, or inflammation and potently activates mast cells. J Exp Med 204, 253-258, doi:10.1084/jem.20062211 (2007).
63 Omori, M. & Ziegler, S. Induction of IL-4 expression in CD4(+) T cells by thymic stromal lymphopoietin. J Immunol 178, 1396-1404 (2007).
64 Byers, D. E. et al. Long-term IL-33-producing epithelial progenitor cells in chronic obstructive lung disease. J Clin Invest 123, 3967-3982, doi:10.1172/JCI65570 (2013).
65 Soumelis, V. et al. Human epithelial cells trigger dendritic cell mediated allergic inflammation by producing TSLP. Nat Immunol 3, 673-680, doi:10.1038/ni805 (2002).
66 Leyva-Castillo, J. M. et al. Skin thymic stromal lymphopoietin initiates Th2 responses through an orchestrated immune cascade. Nat Commun 4, 2847, doi: 10. 103 8/ncomms3 847 (2013).
67 Wambre, E. et al. A phenotypically and functionally distinct human TH2 cell subpopulation is associated with allergic disorders. Sci Transl Med 9, doi:10.1126/scitranslmed.aam9171 (2017).
68 Monticelli, L. A. et al. Innate lymphoid cells promote lung-tissue homeostasis after infection with influenza virus. Nat Immunol 12, 1045-1054, doi:10.1031/ni.2131 (2011).
69 Zaiss, D. M. W., Gause, W. C., Osborne, L. C. & Artis, D. Emerging functions of amphiregulin in orchestrating immunity, inflammation, and tissue repair. Immunity 42, 216-226, doi:10.1016/j.immuni.2015.01.020 (2015).
70 Yoshimura, T., Yoshikawa, M., Otori, N., Haruna, S. & Moriyama, H. Correlation between the prostaglandin D(2)/E(2) ratio in nasal polyps and the recalcitrant pathophysiology of chronic rhinosinusitis associated with bronchial asthma. Allergol Int 57, 429-436, doi:10.2332 allergolint.O-08-545 (2008).
71 Portelli, M. A., Hodge, E. & Sayers, I. Genetic risk factors for the development of allergic disease identified by genome-wide association. Clin Exp Allergy 45, 21-31, doi: 10.1111/cea. 12327 (2015).
72 Ober, C. Asthma Genetics in the Post-GWAS Era. Ann Am Thorac Soc 13 Suppl 1, S85-90, doi:10.1513/AnnalsATS.201507-459MG (2016).
73 Griggs, T. F. et al. Rhinovirus C targets ciliated airway epithelial cells. Respir Res 18, 84, doi:10.1186/s12931-017-0567-0 (2017).
74 Bonnelykke, K. et al. Cadherin-related Family Member 3 Genetics and Rhinovirus C Respiratory Illnesses. Am J Respir Crit Care Med 197, 589-594, doi:10.1164/rccm.201705-10210C (2018).
75 Waikel, R. L., Kawachi, Y., Waikel, P. A., Wang, X. J. & Roop, D. R. Deregulated expression of c-Myc depletes epidermal stem cells. Nat Genet 28, 165-168, doi:10.1038/88889 (2001).
76 Kuperman, D. A. et al. Direct effects of interleukin-13 on epithelial cells cause airway hyperreactivity and mucus overproduction in asthma. Nat Med 8, 885-889, doi:10.1038/nm734 (2002).
77 Gomperts, B. N., Kim, L. J., Flaherty, S. A. & Hackett, B. P. IL-13 regulates cilia loss and foxj 1l expression in human airway epithelium. Am J Respir Cell Mol Biol 37, 339-346, doi: 10.1165/rcmb.2006-04000C (2007).
78 Hackett, N. R. et al. The human airway epithelial basal cell transcriptome. PLoS One 6, e18378, doi:10.1371/journal.pone.0018378 (2011).
79 Gordon, E. D. et al. A protective role for periostin and TGF-beta in IgE-mediated allergy and airway hyperresponsiveness. Clin Exp Allergy 42, 144-155, doi:10.1111/j.1365-2222.2011.03840.x (2012).
80 Butler, A., Hoffman, P., Smibert, P., Papalexi, E. & Satija, R. Integrating single-cell transcriptomic data across different conditions, technologies, and species. Nat Biotechnol, doi:10.1038/nbt.4096 (2018).
81 Barrett, A. J. The cystatins: a diverse superfamily of cysteine peptidase inhibitors. Biomed Biochim Acta 45, 1363-1374 (1986).
82 Takaoka, M. et al. EGF-mediated regulation of IGFBP-3 determines esophageal epithelial cellular response to IGF-I. Am J Physiol Gastrointest Liver Physiol 290, G404-416, doi:10.1152/ajpgi.00344.2005 (2006).
83 Epstein, A. C. et al. C. elegans EGL-9 and mammalian homologs define a family of dioxygenases that regulate HIF by prolyl hydroxylation. Cell 107, 43-54 (2001).
84 Podolsky, D. K., Gerken, G., Eyking, A. & Cario, E. Colitis-associated variant of TLR2 causes impaired mucosal repair because of TFF3 deficiency. Gastroenterology 137, 209-220, doi: 10. 1053/j.gastro.2009.03.007 (2009).
85 McLoughlin, K., Schluter, J., Rakoff-Nahoum, S., Smith, A. L. & Foster, K. R. Host Selection of Microbiota via Differential Adhesion. Cell Host Microbe 19, 550-559, doi:10.1016/j.chom.2016.02.021 (2016).
86 Lafkas, D. et al. Therapeutic antibodies reveal Notch control of transdifferentiation in the adult lung. Nature 528, 127-131, doi:10.1038/nature15715 (2015).
87 Rock, J. R. et al. Notch-dependent differentiation of adult airway basal stem cells. Cell Stem Cell 8, 639-648, doi:10.1016/j.stem.2011.04.003 (2011).
88 Danahay, H. et al. Notch2 is required for inflammatory cytokine-driven goblet cell metaplasia in the lung. Cell Rep 10, 239-252, doi:10.1016/j.celrep.2014.12.017 (2015).
89 Tata, P. R. & Rajagopal, J. Plasticity in the lung: making and breaking cell identity. Development 144, 755-766, doi: 10. 1242/dev. 143784 (2017).
90 Rawlins, E. L. et al. The role of Scgb1a1+Clara cells in the long-term maintenance and repair of lung airway, but not alveolar, epithelium. Cell Stem Cell 4, 525-534, doi:10.1016/j.stem.2009.04.002 (2009).
91 Ostedgaard, L. S. et al. Gel-forming mucins form distinct morphologic structures in airways. Proc Natl Acad Sci USA 114, 6842-6847, doi:10.1073/pnas.1703228114 (2017).
92 Roy, M. G. et al. Muc5b is required for airway defence. Nature 505, 412-416, doi: 10.1038/nature12807 (2014).
93 Basbaum, C. B. & Finkbeiner, W. E. Airway secretion: a cell-specific analysis. Horm Metab Res 20, 661-667, doi:10.1055/s-2007-1010912 (1988).
94 Miller, L. S. & Cho, J. S. Immunity against Staphylococcus aureus cutaneous infections. Nat Rev Immunol 11, 505-518, doi: 10.1038/nri3010 (2011).
95 Musa, M. et al. Differential localisation of BPIFA1 (SPLUNC1) and BPIFB1 (LPLUNC1) in the nasal and oral cavities of mice. Cell Tissue Res 350, 455-464, doi: 10.1007/s00441-012-1490-9 (2012).
96 Adelson, J. W. & Miller, P. E. Heterogeneity of the exocrine pancreas. Am J Physiol 256, G817-825, doi:10.1152/ajpgi.1989.256.5.G817 (1989).
97 Basbaum, C. B., Jany, B. & Finkbeiner, W. E. The serous cell. Annu Rev Physiol 52, 97-113, doi:10.1146/annurev.ph.52.030190.000525 (1990).
98 Pipkorn, U. & Karlsson, G. Methods for obtaining specimens from the nasal mucosa for morphological and biochemical analysis. Eur Respir J 1, 856-862 (1988).
99 El Hadri, K. et al. Thioredoxin-1 promotes anti-inflammatory macrophages of the M2 phenotype and antagonizes atherosclerosis. Arterioscler Thromb Vasc Biol 32, 1445-1452, doi:10.1161/ATVBAHA.112.249334 (2012).
100 Corrotte, M. et al. Ral isoforms are implicated in Fc gamma R-mediated phagocytosis: activation of phospholipase D by RalA. J Immunol 185, 2942-2950, doi: 10.4049/jimmunol.0903 138 (2010).
101 Kawai, T. & Akira, S. The role of pattern-recognition receptors in innate immunity: update on Toll-like receptors. Nat Immunol 11, 373-384, doi:10.1038/ni.1863 (2010).
102 Bethunaickan, R. et al. Identification of stage-specific genes associated with lupus nephritis and response to remission induction in (NZB×NZW) F1 and NZM2410 mice. Arthritis Rheumatol 66, 2246-2258, doi:10.1002/art.38679 (2014).
103 Zhao, F. et al. Disruption of Paneth and goblet cell homeostasis and increased endoplasmic reticulum stress in Agr2−/− mice. Dev Biol 338, 270-279, doi:10. 1016/j.ydbio.2009.12.008 (2010).
104 Bingle, L. et al. WFDC2 (HE4): a potential role in the innate immunity of the oral cavity and respiratory tract and the development of adenocarcinomas of the lung. Respir Res 7, 61, doi:10.1186/1465-9921-7-61 (2006).
105 Kaetzel, C. S., Robinson, J. K., Chintalacharuvu, K. R., Vaerman, J. P. & Lamm, M. E. The polymeric immunoglobulin receptor (secretory component) mediates transport of immune complexes across epithelial cells: a local defense function for IgA. Proc Natl Acad Sci USA 88, 8796-8800 (1991).
106 Corbin, B. D. et al. Metal chelation and inhibition of bacterial growth in tissue abscesses. Science 319, 962-965, doi: 10.1126/science. 1152449 (2008).
107 Chaturvedi, P., Singh, A. P. & Batra, S. K. Structure, evolution, and biology of the MUC4 mucin. FASEB J 22, 966-981, doi:10.1096/fj.07-9673rev (2008).
108 Harrod, K. S., Mounday, A. D., Stripp, B. R. & Whitsett, J. A. Clara cell secretory protein decreases lung inflammation after acute virus infection. Am J Physiol Lung Cell Mol Physiol 275, L924-L930, doi: 10.1152/aj plung. 1998.275.5.L924 (1998).
109 Mouta Carreira, C. et al. S100A13 is involved in the regulation of fibroblast growth factor-1 and p40 synaptotagmin-1 release in vitro. J Biol Chem 273, 22224-22231 (1998).
110 Zhu, J. et al. Conversion of proepithelin to epithelins: roles of SLPI and elastase in host defense and wound repair. Cell 111, 867-878 (2002).
111 Giovannini-Chami, L. et al. Distinct epithelial gene expression phenotypes in childhood respiratory allergy. Eur Respir J 39, 1197-1205, doi: 10.1183/09031936.00070511 (2012).
112 Naeem, S., Thompson, L. J., Lawler, S. P., Lawton, J. H., Woodfin, R. M. . Declining biodiversity can alter the performance of ecosystems. Nature 368, 734-737, doi:doi:10.1038/368734a0 (1994).
113 Simpson, E. H. Measurement of Diversity. Nature 163, 688-688, doi:doi:10.1038/163688a0 (1949).
114 Olender, T. et al. The human olfactory transcriptome. BMC Genomics 17, 619, doi:10.1186/s12864-016-2960-3 (2016).
115 Wang, W. et al. Transcriptome Analysis Reveals Distinct Gene Expression Profiles in Eosinophilic and Noneosinophilic Chronic Rhinosinusitis with Nasal Polyps. Sci Rep 6, 26604, doi: 10.1038/srep26604 (2016).
116 Lorts, A., Schwanekamp, J. A., Baudino, T. A., McNally, E. M. & Molkentin, J. D.
Deletion of periostin reduces muscular dystrophy and fibrosis in mice by modulating the transforming growth factor-beta pathway. Proc Natl Acad Sci USA 109, 10978-10983, doi:10.1073/pnas. 1204708109 (2012).
117 Di, W. L., Hennekam, R. C., Callard, R. E. & Harper, J. I. A heterozygous null mutation combined with the G1258A polymorphism of SPINK5 causes impaired LEKTI function and abnormal expression of skin barrier proteins. Br J Dermatol 161, 404-412, doi:10.1111/j.1365-2133.2009.09231.x (2009).
118 Nusse, R. & Clevers, H. Wnt/beta-Catenin Signaling, Disease, and Emerging Therapeutic Modalities. Cell 169, 985-999, doi:10.1016/j.cell.2017.05.016 (2017).
119 Reuter, S., Beckert, H. & Taube, C. Take the Wnt out of the inflammatory sails: modulatory effects of Wnt in airway diseases. Lab Invest 96, 177-185, doi:10. 1038/labinvest.2015.143 (2016).
120 Wagner, T. E., Frevert, C. W., Herzog, E. L. & Schnapp, L. M. Expression of the integrin subunit alpha8 in murine lung development. J Histochem Cytochem 51, 1307-1315, doi:10.1177/002215540305101008 (2003).
121 Levine, D. et al. Expression of the integrin alpha8betal during pulmonary and hepatic fibrosis. Am J Pathol 156, 1927-1935 (2000).
122 Haghverdi, L., Buttner, M., Wolf, F. A., Buettner, F. & Theis, F. J. Diffusion pseudotime robustly reconstructs lineage branching. Nat Methods 13, 845-848, doi:10.1038/nmeth.3971 (2016).
123 Buenrostro, J. D. et al. Single-cell chromatin accessibility reveals principles of regulatory variation. Nature 523, 486-490, doi:10.1038/nature14590 (2015).
124 Corces, M. R. et al. An improved ATAC-seq protocol reduces background and enables interrogation of frozen tissues. Nat Methods, doi:10. 1038/nmeth.4396 (2017).
125 Dekker, J. & Mirny, L. The 3D Genome as Moderator of Chromosomal Communication. Cell 164, 1110-1121, doi:10.1016/j.cell.2016.02.007 (2016).
126 Bernstein, B. E. et al. A bivalent chromatin structure marks key developmental genes in embryonic stem cells. Cell 125, 315-326, doi:10.1016/j.cell.2006.02.041 (2006).
127 Kim, K. et al. Epigenetic memory in induced pluripotent stem cells. Nature 467, 285-290, doi: 10.1038/nature09342 (2010).
128 Ostuni, R. et al. Latent enhancers activated by stimulation in differentiated cells. Cell 152, 157-171, doi:10.1016/j.cell.2012.12.018 (2013).
129 Lambert, S. A. et al. The Human Transcription Factors. Cell 172, 650-665, doi:10.1016/j.cell.2018.01.029 (2018).
130 Shaulian, E. & Karin, M. AP-1 as a regulator of cell life and death. Nat Cell Biol 4, E131-136, doi:10.1038/ncb0502-e131 (2002).
131 Wan, H. et al. Compensatory roles of Foxal and Foxa2 during lung morphogenesis. J Biol Chem 280, 13809-13816, doi:10.1074/jbc.M414122200 (2005).
132 Thompson, M. R., Xu, D. & Williams, B. R. ATF3 transcription factor and its emerging roles in immunity and cancer. J Mol Med (Berl) 87, 1053-1060, doi:10.1007/s00109-009-0520-x (2009).
133 Sur, I., Rozell, B., Jaks, V., Bergstrom, A. & Toftgard, R. Epidermal and craniofacial defects in mice overexpressing Klf5 in the basal layer of the epidermis. J Cell Sci 119, 3593-3601, doi: 10.1242/jcs.03070 (2006).
134 Yang, A. et al. p63 is essential for regenerative proliferation in limb, craniofacial and epithelial development. Nature 398, 714-718, doi:10.1038/19539 (1999).
135 Lopez-Bergami, P., Lau, E. & Ronai, Z. Emerging roles of ATF2 and the dynamic AP1 network in cancer. Nat Rev Cancer 10, 65-76, doi:10.1038/nrc2681 (2010).
136 Sarkar, A. & Hochedlinger, K. The sox family of transcription factors: versatile regulators of stem and progenitor cell fate. Cell Stem Cell 12, 15-30, doi:10.1016/j.stem.2012.12.007 (2013).
137 Stehling-Sun, S., Dade, J., Nutt, S. L., DeKoter, R. P. & Camargo, F. D. Regulation of lymphoid versus myeloid fate ‘choice’ by the transcription factor Mef2c. Nat Immunol 10, 289-296, doi: 10.1038/ni. 1694 (2009).
138 Senoo, M., Pinto, F., Crum, C. P. & McKeon, F. p63 Is essential for the proliferative potential of stem cells in stratified epithelia. Cell 129, 523-536, doi:10.1016/j.cell.2007.02.045 (2007).
139 Fulcher, M. L., Gabriel, S., Burns, K. A., Yankaskas, J. R. & Randell, S. H. Well-differentiated human airway epithelial cell cultures. Methods Mol Med 107, 183-206 (2005).
140 Rajavelu, P. et al. Airway epithelial SPDEF integrates goblet cell differentiation and pulmonary Th2 inflammation. J Clin Invest 125, 2021-2031, doi:10.1172/JCI79422 (2015).
141 Woodruff, P. G. et al. Genome-wide profiling identifies epithelial cell genes associated with asthma and with treatment response to corticosteroids. Proc Natl Acad Sci USA 104, 15858-15863, doi:10.1073/pnas.0707413104 (2007).
142 Netea, M. G. et al. Trained immunity: A program of innate immune memory in health and disease. Science 352, aaf1098, doi:10.1126/science.aafl098 (2016).
143 Gosselin, D. et al. An environment-dependent transcriptional network specifies human microglia identity. Science 356, doi:10.1126/science.aa13222 (2017).
144 Mercurio, S., Latinkic, B., Itasaki, N., Krumlauf, R. & Smith, J. C. Connective-tissue growth factor modulates WNT signalling and interacts with the WNT receptor complex. Development 131, 2137-2147, doi:10.1242/dev.01045 (2004).
145 Boscke, R. et al. Wnt Signaling in Chronic Rhinosinusitis with Nasal Polyps. Am J Respir Cell Mol Biol 56, 575-584, doi: 10.1165/rcmb.2016-00240C (2017).
146 Sanchez-Tillo, E. et al. beta-catenin/TCF4 complex induces the epithelial-to-mesenchymal transition (EMT)-activator ZEB 1 to regulate tumor invasiveness. Proc Natl Acad Sci USA 108, 19204-19209, doi:10.1073/pnas.1108977108 (2011).
147 Wenzel, S. et al. Dupilumab in persistent asthma with elevated eosinophil levels. N Engl J Med 368, 2455-2466, doi:10.1056/NEJMoa1304048 (2013).
148 Yan, L. et al. Activation of the canonical Wnt/beta-catenin pathway in ATF3-induced mammary tumors. PLoS One 6, e16515, doi:10.1371/journal.pone.0016515 (2011).
149 Lambrecht, B. N. & Hammad, H. The immunology of the allergy epidemic and the hygiene hypothesis. Nat Immunol 18, 1076-1083, doi:10.1038/ni.3829 (2017).
150 Bachert, C. et al. Effect of Subcutaneous Dupilumab on Nasal Polyp Burden in Patients With Chronic Sinusitis and Nasal Polyposis: A Randomized Clinical Trial. JAMA 315, 469-479, doi:10.1001/jama.2015.19330 (2016).
151 Rochman, M. et al. Neurotrophic tyrosine kinase receptor 1 is a direct transcriptional and epigenetic target of IL-13 involved in allergic inflammation. Mucosal Immunol 8, 785-798, doi:10.1038/mi.2014.109 (2015).
152 Beyaz, S. et al. High-fat diet enhances stemness and tumorigenicity of intestinal progenitors. Nature 531, 53-58, doi: 10.1038/nature17173 (2016).
153 Lindemans, C. A. et al. Interleukin-22 promotes intestinal-stem-cell-mediated epithelial regeneration. Nature 528, 560-564, doi:10.1038/nature 16460 (2015).
154 Naik, S. et al. Inflammatory memory sensitizes skin epithelial stem cells to tissue damage. Nature, doi:10.1038/nature24271 (2017).
155 Dai, X. & Medzhitov, R. Inflammation: Memory beyond immunity. Nature 550, 460-461, doi: 10.1038/nature24154 (2017).
156 Meltzer, E. O. et al. Rhinosinusitis: establishing definitions for clinical research and patient care. J Allergy Clin Immunol 114, 155-212, doi:10.1016/j.jaci.2004.09.029 (2004).
157 Dhariwal, J. et al. Mucosal Type 2 Innate Lymphoid Cells Are a Key Component of the Allergic Response to Aeroallergens. Am J Respir Crit Care Med 195, 1586-1596, doi:10.1164/rccm.201609-18460C (2017).
158 Proud, D., Sanders, S. P. & Wiehler, S. Human rhinovirus infection induces airway epithelial cell production of human beta-defensin 2 both in vitro and in vivo. J Immunol 172, 4637-4645 (2004).
159 Beck, L. A. et al. Dupilumab treatment in adults with moderate-to-severe atopic dermatitis. NEngl J Med 371, 130-139, doi:10.1056/NEJMoa1314768 (2014).
160 Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
161 McDavid, A. et al. Data exploration, quality control and testing in single-cell qPCR-based gene expression experiments. Bioinformatics 29, 461-467, doi:10.1093/bioinformatics/bts714 (2013).
162 Trombetta, J. J. et al. Preparation of Single-Cell RNA-Seq Libraries for Next Generation Sequencing. Curr Protoc Mol Biol 107, 4 22 21-24 22 17, doi:10.1002/0471142727.mb0422s107 (2014).
163 Dobin, A. et al. STAR: ultrafast universal RNA-seq aligner. Bioinformatics 29, 15-21, doi:10.1093/bioinformatics/bts635 (2013).
164 Li, B. & Dewey, C. N. RSEM: accurate transcript quantification from RNA-Seq data with or without a reference genome. BMC Bioinformatics 12, 323, doi:10.1186/1471-2105-12-323 (2011).
165 Love, M. I., Huber, W. & Anders, S. Moderated estimation of fold change and dispersion for RNA-seq data with DESeq2. Genome Biol 15, 550, doi: 10.1186/s13059-014-0550-8 (2014).
166 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for modulating cellular interactions within cellular ensembles, comprising
administering to a cellular ensemble a modulating agent in an amount sufficient to change or modify extracellular signaling from a first cell type such that a change in one or more cell states is induced in a second cell type.

2. The method of claim 1, wherein the cellular ensemble is a two dimensional (2D) or three dimensional (3D) in vitro or ex vivo culture, a tissue on a chip, an organoid, or in vivo cells within a defined tissue, tissue compartment, or signaling microenvironment.

3. The method of claim 2, wherein the first cell type comprises one or more pathogenic cells and the second cell type comprises on or more host cell types.

4. The method of claim 2, wherein the first cell type comprises one or more diseased cell types and the second cell type comprises one or more healthy cell types.

5. The method of claim 4, wherein the one or more diseased cell types are cancer cells.

6. The method of claim 2, wherein the first cell type comprises one or more stem cell types and the second cell type comprises one or more immune cell types.

7. The method of claim 1, wherein extracellular signaling is receptor-ligand mediated, cytokine/chemokine mediated, or metabolically mediated.

8. The method of claim 1, wherein the cellular ensemble comprises epithelial tissues.

9. The method of claim 8, wherein the first cell type is an immune cell and the second cell type is an epithelial stem cell.

10. The method of claim 9, wherein the epithelial stem cell is a basal cell.

11. The method of claim 9, wherein the modulating agent antagonizes IL-4/13 signaling in the immune cells such that the differentiation of basal cells is induced.

12. A method of treating inflammatory disease in barrier tissues comprising administering to a subject in need thereof, an IL-4 and/or IL-13 modulating agent in an amount sufficient to induce basal cell differentiation.

13. The method of claim 11, wherein the modulating agent blocks IL-4 and/or IL-13 signaling.

14. The method of claim 11, wherein the modulating agent reduces or blocks induction of CTNNB1/Wnt.

15. The method of claim 13, further comprising administration of a second modulating agent, wherein the second modulating agent targets one or more gene or gene products selected from the group consisting of:
a) FAM3C and CTNNB1; or
b) PHAX, LPP, PDE6A, MEFV, TVP23C, PLCXD1, PHACTR4, AP1S3, TRAF3IP2, PGAM5, SMYD4, UGGT1, DFFA, UBB, LOC284454, IFITM3, TUBB2A, IRF1, AXL, SPN, RNF41, ANGPTL4, TUBB2B, NMNAT1, TANGO2, HOOK3, S1PR2, HLA-H, LRTOMT, ZNF430, SCOC, ZNF793, CYP20A1, MDM2, KREMEN1, ZC3H12D, FBLIM1, LOC646214, PDDC1, HLA-E, KRT6C, HYDIN2, COX18, PPM1K, MBOAT1, LINC00294, ZNF526, P2RX5-TAX1BP3, GNE, NUDT19, KIF18B, PDE4C, LETM1, TLCD2, CHST6, METTL21A, IKZF3, POTEE, HLA-G, XPNPEP3, TMEM33, MOG, TSPYL1, RBMS2, TAPBP, ANKRD20A9P, STON2, CYP4V2, POTEM, OPA3, POLH, ZNF805, IFITM2, C17orf75, IRGQ, KCNQ1OT1, TAF8, UGDH-AS1, CCL5, NOL9, CHP1, ORAI2, CA5B, HLA-B, LYZ, TOR1AIP2, TRAPPC2, SGTB, ZNF264, XIAP, RAMP2-AS1, SCAI, ZBTB3, ZNF490, ORC4, DNAL1, FBXL18, CPT1A, TNFAIP8L1, LRRC57, RBM3, HLA-C, LOC148709, LYRM7, ATCAY, PRICKLE2-AS3, AGMAT, NF2, LOC100131257, ACTG1, RUNDC1, MAVS, RPL36A-HNRNPH2, PNPO, GDPD1, ILF3-AS1, LOC284023, PRR11, TMEM41B, ZBTB8A, TUBB4A, ZNF850, VHL, IVD, FOXK1, MDM4, CCDC142, TRPV1, UBC and SENP5; or
c) TM4SF1, IL1B; or
d) CD44, CTNNB1, POSTN and TNC; or
e) CD44, ANXA2, YBX1, EDN1, ARPC2, VAMP8, RPS18, EHF, HAS3, MBNL2, MTRNR2L9, CPXM2, NOS2, HMGB3, TNC, IFITM3, TSPAN3, BIK, CXADR, LGALS7, SERPINFI, TPM4, SERINC5, IGHG4, SERPINE2, NTRK2, PAPSS2, SDPR, ANXA3, RPL21, POSTN, CXCL2, MTRNR2L1, NDRG1, TPI1, SERPINB4, WIPI1, AL353147.1, CKB, GCLM, LRRC17, FAM110C, RPL18A, CAV1, VAMP5, IGHA1, VSNL1, KCNE3, SPARCL1, ARID5B, ARL4C, MTRNR2L3, CXCL1, IGHG1, CNN3, CST1, MYO10, IGHG3, AAMDC, HSPH1, ENO1, VCL, SYNGR2, LAMTOR4, CCL26, TSLP, SERPINB2, TMEM123, TGIF1, IGHM, RPL7, CMYA5, SH3BGRL3, LOXL4, MAP3K8, ANO1, PGAM1, STOM, MYL9, PLS1, CTNNAL1, EFNA1, MTRNR2L8, RPL13A, PAWR, RPL41, LDLR, ITGB1, TCEB2, SERPINB10, NEDD9, SERPINB13, LDHA, DSE, DDX58, GPC3, SERF2, KLHL5, MRPS6, ADAM9, CDCP1, EGLN3, SMARCAD1, CTNNB1, MIF, PFN1, CTD-2228K2.5, CDH26, IGHA2, ST6GAL1, PLEKHA1, RPS25, GALNT7, CTD-2090113.1, SNRPG, RPL17, SRGN, ACTN1, MTRNR2L13, MORF4L1, RPSAP58, AREG, PLEKHA5, TMSB4X, IFITM1, SLC5A3, GEM, REXO2, KCNJ16, RPS4Y1, MMP10, NRG1, TBC1D9, LBH, FBXO32, TXNDC17, PHLDB2, SLC6A14, KRT6A, CP, TNFAIP3, CTD-2319112.1, SERTAD4-AS1, AMD1, FOSL1, LRRFIP1, IL6, UBBP4, BTG3, SLC2A1, IGJ, TMA7, SSR4, FGFBP1, HSD17B13, CCDC51, SMOC2, SHISA5, CTSC and XPR1; or
f) HLA-F, SOX4, MYL12B, BST2, SHC1, INHBA, IF1 6, SQSTM1, LYPLA1, RAP1B, MT1L, ANXA8L1, PLTP, NBPF10, MGST3, PLEKHB2, ICAM1, PHLDA1, PPP1CB, PAFAH1B2, SLC3A2, ANXA8L2, ISG20, GNG12, TNFRSF10B, GRN, DSG3, FN1, IF127, C6orf62, SERPINE1, BNIP3, HLA-L, AKIRIN1, SYPL1, EFNA1, CDKN1A, TUBA1A, HLA-A, CCND2, CACUL1, RPS26, B2M, FKBP9L, AHNAK2, IFITM1, TFPI2, RPS4Y1, SNAI2, KRT6A, SRP9, CTGF and PGK1; or
g) CDH3, MYO1B, HAS3, EHF, TNC, DKK3, SERPINB4, FABP5, TNFSF10, PTHLH, TBL1XR1, GJB2, CAV1, THBS1, TNS4, SHISA9, LOC100132247, KRT18, LITAF, HSPH1, CCL26, CSF3, KRT8, SPHK1, HK1, CDH6, SERPINB3, TP63, KRT23 and CTSC; or
h) ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4 and EGR1; or
i) ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4, EGR1, GLUL, ANXA1, EGR3, JUN, ID1, LMNA, JUNB, KRT17, HSPA1B, PPP1R15A, TSC22D1, SERPINE1, ETS2, DNAJB1, MTSS1L, ERRFI1, KRT5, TIPARP, LGALS3, SERTAD1, TSC22D3, PTP4A1, BPIFB1, MAFF, MUC5AC, FOSB, PMAIP1, HSPA8, ZFP36L2, IER2, NFKBIA, CDKN1A, BRD2, RPS16, CAPS, EZR, FAM107B, KRT19, F3, KLF4, RHOB, FOSL2, GPRC5A, SOCS3, TRIB1, MIR22HG, HSP90AA1, PRDX1, WEE1, FTL, SLC25A25, VMP1, DDIT3, RND3, ATP5G2, TACSTD2, HBEGF, CD55, EMP1, MCL1, CEBPD, FKBP5, YPEL5, ACTG1, C12orf57, KAL1, RN7SK, NR1D2, FXYD3, KLF5, FOS, ANKRD37, MIDN, PER2, STARD7, WFDC2, DUSP6, HSPB1, SEPW1, MYC, CXCL17, KLF9, SYT8, GPX4, MIR24-2, EPS8, AHNAK, JUP, ADH7, S100P, POLR2A, PPP1R10, GCNT1, GAPDH, IER3, HOOK1, HN1L, TOMM20, SCGB1A1, GSR, SERPINB5, S100A6, PEBP1, CDC42EP4, AQP3, ADRB2, DDIT4, PER1, ADM, IRF1, PRDX6, RPS14, TPT1, PLXNB1, BHLHE40, SQSTM1, SCP2, KRT15, SGK1, ACTB, NABP1, IGFBP3, PPP1R15B, SOD2, SOX9, CTSH, MXD1, THBS1, CIB1, RAP2B, NEDD4L, RAB34, PRDX5, ANAPC11, RAC1, ARF1, HS3ST1, MAOA, HLA-E, EID1, BAG5, SERPINH1, CITED2, AJUBA, CTSK, PAFAH1B2, GNB2L1, LMO7, PDPN, UBE3A, HNRNPAB, GSTP1, TM4SF1, PIM3, DLX5, C9orf3, HPGD, ANXA11, UBC, PLP2, EGR2, S100A10, LAMA5, ALOX15, MUC16, RPL4, TNS4, TUBB, RHOC, CYP2S1, PPAP2C, GPX1, QARS, CSNK1D, HINT1, PDLIM1, BLVRB, PSMB6, TSPO, CDCA7L, HNRNPF, RPS4X, EPB41L4A-AS1, IRF6, FSTL1, FTH1, UBL5, ADI1, PHLDA1, IFITM2, VAPA, PPP1CB, EIF4A3, UBB, SELM, NFKBIZ, UBR5, PTPN14, RAN, PPP2CA, CKS2, ELF3, EIF4A1, CSTB, TSPYL2, PLEC, UGDH, IBTK, TUBA1C, HLA-A, PHYHDI, RNA28S5, UBA52, RPL30, FLOT1, CHST9, TMEM14B, EIF1, PTHLH, STAT6, LRRC8A, MTRNR2L2, VPS37B, HN1, ELK4, NBN, MID1, RAB2A, CCND2, PSMB1, KDSR, SIK1, KDM2A, CDC42SE2, SLC35F5, CLTA, SF3B14, TUBAIB, C6orf48, SRSF3, RPS12, ARHGAP18, C16orf72, ALDOA, MRFAP1, LYPLAL1, JUND, TRAK1, MINK1, RPL11, HMGN2, LNX2, CFL1, TAGLN2, SPRY2, HLA-C, SDC4, EEF2, PCBP1, PER3, RPLP1, RPL8, REV1, S100A2, NDUFV1, EIF3K, MYADM, DDX3Y, KRT8, SLPI, CHD4, SEMA5A, TOR1AIP2, CHD2, GSN, GDI2, HLA-B, FLRT3, PERP, DNAJA1, OBSCN, TNFRSF1A, PSMB3, KLF6, WDR26, TFCP2L1, ZCRB1, LITAF, SNRNP70, EIF3I, SPINT1, FLOT2, SLC3A2, UGT2A2, ID2, TMEM59, HMGA1, CTNND1, FKBP9, TUBB4B, POLR2G, SLC9A3R1, SNHG5, HEBP2, RNF181, BCAM, SRI, ARHGDIA, HERPUDI, KLF10, MAT2A, LEPROT, SPCS1, SORL1, CCNL1, CYB5R3, USP22, MKNK2, SEC14L1, SEC61B, BTF3, FZD6, DAZAP2, SFN, MGST1, LAMB3, TNFRSF12A, OAT, CD99, CAV2, FLNA, STXBP3, RIPK4, SRSF7, EIF3D, TNPO1, MYL12B, GCLC, METTL7A, RPL28, F2R, RNA18S5, TMEM261, SOD1, YBX3, C1 lorf31, MYL6, ELOVL5, ATOX1, RPLP2, FBXL5, MAP3K13, MDK, TOB1, DLL1, HSP90AB1, SNCA, LAMA3, CSNK1A1, ATP1B3, RPS2, EIF4H, UBE2D3, CEBPB, TGFBR2, RPS3, GADD45B, GAS5, ZNHIT1, DDR1, JAG2, WNT4, NDFIP1, APH1A, OAZ1, HDAC7, ATP1A1, FAU, PPL, RAB11A, NCOA4, ATP50O, SF1, UQCRQ, NONO, FBXL3, ID3, DHCR24, IER5, OPTN, TMSB10, PPP1CA, ARPC5, RAB1A, TSKU, RPL19, BTG1, NACA, RAI14, CAPN1, 10-Sep, CDKN2AIP, PHB2, DDOST, S100A16, BLCAP, CSDE1, EDF1, CDK5RAP2, PRKAR1A, CNN2, FGFR3, ALDH3A2, TMX4, RPL7A, RPS27, C14orf166, RPL18, C7orf55-LUC7L2, GNG5, GHITM, IVNS1ABP, H3F3B, RBM3, RPL27, SMIM15, ARFGAP3, PPP4R1, NDUFB4, SSBP1, RPS21, DDB1, ALAS1, SERINC3, TMBIM6, AQP5, RPS5, EIF3F, 7-Mar, SDF4, RPL15, CDC42SE1, LGALS8, PAK1, NAMPT, EGFR, NDUFS1, GPR155, RAD23B, CD9, LIMK2, DLK2, CBR1, PPP2R5C, PRRC2C, RPS11, SH3GLB1, HNRNPAO, NUMA1, FGFR2, GABARAPL2, ETV3, H3F3C, SSH1, ATF4, SH3BGRL, ABHD2, ZFYVE21, RPS24, SYNCRIP, DDX17, RPS8, AFG3L2, RPL26, HNRNPA3, VPS26A, CHD8, RPS19, NCL, PDCD10, DST, POMP, APP, CLU, SRP9, SKP1, RPL41, RPL39, UQCRB, MTUS1, IFT81, USP8, RPL6, RNF19A, ADAM28, LSM3, ASPH, GOLGB1, HNRNPH1, CP, TPM3, EEF1D, PDIA4, PTMA, CMYA5, PGAP1, MPHOSPH6, MTRNR2L8, MALAT1, VMO1, ITGAV, PDIA3, TLK1, RPL21, MTRNR2L10, CXADR, SPCS2, HSPA5, POSTN, LARS, MORF4L1, ATPSI, TNC, AKAP9, TMSB4X, BPTF, SESN3, SYNE2, TTC3, C1R, RPL10, MT-ND6, SERPINB4, PHPT1, RPL34, IGFBP2, MTRNR2L1, SLC38A1, SMC6, ENAH, SON, HLA-DRA, YWHAE, HSP90B1, MTRNR2L7, MTRNR2L5, MTRNR2L11, MT-ND2, MTRNR2L3, CTC-338M12.5, MTRNR2L13, MMP10, MT-RNR1, IGHA1, IGJ, MT-ATP8, MT-ND1, MT-ND5, MT-RNR2, MT-CO2, MT-CYB, MT-ATP6, MT-ND4L, MT-CO3, IGKC, MT-ND4, and MT-CO1; or
j) SERPINB3, SERPINB4, FABP5, CTSC, GJB2, SPHK1, MBOAT1, ORAI2, KCNQ1OT1, PDDC1, CHP1, CCL5, CYP20A1, IGFBP7, LRTOMT, TOR1AIP2, XPNPEP3, LOXL4, TRAPPC2, ZNF490, GNE, LOC100131257, ZBTB8A, TAPBP, TNFAIP8L1, UGDH-AS1, CCL26, CDH6, IRF1, LOC646214, TNS4, PRICKLE2-AS3, KRT16, PRR11, RBMS2, LYZ, FAM3C, NOL9, POLH, DFFA, PPM1K, UBB, METTL21A, CAV1, IKZF3, PDE4C, SHISA9, AP1S3, HAS3, IRGQ, XIAP, FBLIM1, PHACTR4, SPN, C17orf75, HLA-B, HLA-C, KRT23, NMNAT1, HOOK3, KRT75, THBS1, CCDC142, HYDIN2, LETM1, CAV2, RUNDC1, TLCD2, PLCXD1, HLA-E, UGGT1, UBC, ZNF793, LOC643406, TNC, PNPO, FBXL18, EHF, ZNF264, SMYD4, LEPREL4, RPL36A-HNRNPH2, LRRC57, ANKRD20A9P, VHL, CDH3, ZNF850, MOG, TAF8, RNF41, GDPD1, KRT8, COTL1, PNPT1, ORC4, TRAF3IP2, NUDT19, RNF125, IVD, ODF2L, BTG1, OR7D2, MEFV, MDM4, CTNNB1, OPA3, LINC00294, ZNF526, CRX, CYP4V2, GLTP, KIF18B, SPREDI, MYO1B, ILF3-AS1, TANGO2, TMEM41B, SCAI, LYRM7, TRPV1, CYCS, TUBB2A, CA5B, ATCAY, ASTN2, SLC16A3, POTEE, PDE6A, P2RX5-TAX1BP3, TUBA4A, DDX51, CPT1A, TMEM212, KREMEN1, GPR82, S1PR2, SENP5, PHAX, TUBB4B, ZBTB3, TVP23C, PXMP4, WDR92, OPHN1, AP4S1, LOC284023, L2HGDH, TBC1D24, MTFMT, SGTB, PGAM5, SLFNL1-AS1, IFNLR1, GJC1, RAMP2-AS1, FKBP14, MDM2, KRT6C, AGMAT, CHST6, NWD1, MAVS, AKIP1, FOXK1, METTL2A, FDPSL2A, HLA-G, TFDP2, VSIG1, ZNF483, PTCHD4, SLC6A4, ZNF805, DNAL1, LOC284454, CD3EAP, PIGX, C10orf32, COX18, HLA-H, CPM, FBXO27, PALLD, LPP, NXN, NMT2, NPIPL3, PARD6B, VPS53, FUT1, GREB1, C21orf62, TINAGL1, WDR55, ZC3H12D, DTX3L, NF2, GATAD1, TRPM7, QPCTL, CFLAR, AXL, ALDH1A3, ARPP19, ZNF814, PSTPIP2, LOC90834, MREG, MPPE1, ZFP14, NT5DC3, SLC35F6, MAP1LC3C, PER2, UTP11L, TMEM136, KIN, NPHS1, GK5, PTHLH, LOH12CR2, GNL3L, MXRA7, TUBB4A, TSPYL1, POTEM, FBXL20, TBL1XR1, HP1BP3, TMEM33, PTRF, MYLK3, IFITM3, CABP4, LINC00338, ICA1L, MRI1, EMX2OS, IRAK4, IBA57, LOC728558, POU5F1, ZKSCAN3, PCDH11X, STON2, SLC50A1, LOC613037, AKAP5, ZNF430, ZNF587, EMP2, SLC5A5, STK4, RAC1, ARGFX, MCTS1, MAPK13, ULK2, CYB5R3, PEX13, LOC100132247, LOC100506190, MS4A10, PPP1CC, LIMS1, DCAF10, TBXA2R, ANAPC16, CD84, LOC284950, ACTG1, DLEU2, TRIM45, CCBE1, MED18, ME2, SLC28A2, ZNF738, DAND5, TTC39C, WHAMM, TMEM120B, FLVCR1, SMU1, CEP104, GTF2H3, FXN, IFITM2, RPS2, NME1-NME2, LOC727896, ZYG11B, LOC100507173, MTCH1, ATXN3, NLRP12, TUBB2B, ENPP1, MRPS16, SHOX, SLC35E3, GCLM, EDARADD, ESYT2, C12orf65, CNNM3, BRIP1, GOLGA3, FAM227A, TM7SF3, FCAR, DBT, CACNG8, ANPEP, LOC100287792, SLC12A6, LITAF, INMT, GTF2H2C, SIX4, CYP51A1, ALG1, SPIB, LRPAP1, DKK3, PRRG4, SNHG16, ARSA, RABL5, PACS2, DNAJC22, RELL1, LOC100289019, SCAMP4, KRT18, ZXDC, MOB3A, EXPH5, PCBD2, LOC286437, ANGPTL4, ABCC9, SNIP1, AFMID, Cllorf58, GEMIN8, LOC148709, PTGIS, ZNF785, RNF168, SHROOM1, ZSCAN29, CARD8, FAM122C, FAM73A, SDE2, HSPH1, NLRC3, HAUS3, IAPP, SPAST, PNMA2, LRRC58, LOC100506746, GFOD2, UBE2Q2P1, APOL1, LRP10, CORO2A, ZSCAN22, TUBB6, MIR143HG, HNF1A-AS1, ZNF865, SPATS2, SLC25A32, CCL22, LOC100505876, ADAMTS4, RPS6KA3, TRIM16L, PDLIM5, MPL, ENTPD4, ADAT1, SAR1B, UCKL1-AS1, CYP1A2, METTL2B, ASB6, CEACAM22P, ABL2, LRRC27, ENAH, TP63, ZFAND5, LOC100506688, AK3, PPIEL, OCLN, WAC, SPATA5, TNFSF10, SCOC, LRRN4CL, BHMT2, PTAFR, GSTM3, FKBP5, TNFAIP8L2-SCNM1, PDK3, ZNF714, CXorf56, TRMT2B, CBFA2T2, SLC35E2, MTDH, EEF2K, HAUS2, TPMT, CEP68, SLC4A8, TSIX, CXADR, KLRD1, TMEM165, IDS, SS18, EFNB1, APOBEC3F, ADRA1A, SLC25A15, CLCC1, CBX5, STYX, RBBP5, GNG4, RBM34, ZNF829, RBM3, JAK3, FZD3, ZYG11A, ARHGAP1, PLEKHG2, NUP43, CXorf38, TRIM58, ZNF818P, SKA1, MTPAP, Clorf174, MFSD11, MAPK1IP1L, GATM-AS1, DESI1, IVNS1ABP, GLT25D1, C4orf19, GLG1, RFT1, ZNF626, LPIN3, CSF3, GPR155, SSR1, FCF1, ZNF737, NDUFV3, ATP1A1, EIF2S3, ATP6V1G1, TADA3, CLSPN, TBC1D15, RAB27A, HK1, RTCA, ARNTL2, KCNA7, SMIM12 and ZSWIM1; or
k) IF127, HLA-A, HLA-B, HLA-C, HLA-H, IFITM3, RPS26, ICAM1, IFITM1, IF1 6, UBC, SERPINE1, TFPI2, EFNA1, TUBB2A, HLA-E, HLA-G, AHNAK2, TUBA1A, TUBB2B, POTEE, B2M, FN1, MBOAT1, TAPBP, KRT6C, RPS4Y1, TUBB4A, ORAI2, TORIAIP2, SQSTM1, MT1L, RPL36A-HNRNPH2, TSPYL1, IFITM2, PDDC1, UBB, CHP1, CCL5, LYPLA1, NBPF10, ZBTB8A, PPP1CB, KCNQ1OT1, CYP20A1, ZNF490, IL1B, ANXA8L1, XPNPEP3, CTNNB1, LRTOMT, SHC1, STON2, LYZ, UGDH-AS1, POTEM, MDM2, NOL9, HLA-F, IKZF3, TNFAIP8L1, PPM1K, SLC3A2, AP1S3, PRICKLE2-AS3, DFFA, POLH, IRF1, PAFAH1B2, PLEKHB2, XIAP, UGGT1, CTGF, LOC100131257, TRAPPC2, PDE6A, FBLIM1, PDE4C, PRR11, PNPO, C17orf75, SRP9, RBMS2, CCDC142, AXL, ZNF793, KRT6A, TMEM41B, RAP1B, GNE, RBM3, FOXK1, INHBA, NMNAT1, IRGQ, TLCD2, BST2, NF2, TRAF3IP2, CA5B, SENP5, NUDT19, LINC00294, HOOK3, CYP4V2, LOC646214, CACUL1, PHACTR4, VHL, ZNF526, ZNF264, RUNDC1, HYDIN2, FBXL18, SPN, GDPD1, PHAX, TAF8, LRRC57, METTL21A, IVD, MGST3, SMYD4, ANXA8L2, PLTP, TVP23C, TM4SF1, ZBTB3, FKBP9L, MDM4, C6orf62, ILF3-AS1, TRPV1, LETM1, CHST6, TNFRSF10B, S1PR2, KIF18B, CPT1A, MOG, RNF41, SOX4, PLCXD1, PGAM5, LPP, GNG12, MEFV, ZNF850, OPA3, SCAI, TMEM33, LYRM7, SNAI2, SCOC, DSG3, LOC148709, SGTB, GRN, ATCAY, SYPL1, KREMEN1, BNIP3, ANGPTL4, RAMP2-AS1, MAVS, ZC3H12D, P2RX5-TAX1BP3, CDKN1A, ISG20, ANKRD20A9P, ZNF805, LOC284454, HLA-L, AGMAT, AKIRIN1, MYL12B, TANGO2, ACTG1, LOC284023, COX18, ORC4, PHLDA1, DNAL1, FAM3C, PGK1, ZNF430 and CCND2.

16. The method of claim 12 or 13, wherein the subject suffers from a chronic human inflammatory disease.

17. The method of claim 16, wherein the chronic human inflammatory disease is characterized by basal cell hyperplasia.

18. The method of claim 16, wherein the chronic human inflammatory disease comprises a Type 2 immunity response.

19. The method of claim 16, wherein the chronic human inflammatory disease is chronic rhinosinusitis.

20. The method of any one of claims 12 to 19, wherein the subject in need thereof is selected based on the presence of an IL4/13 signature in on or more epithelia cell types.

21. The method of claim 20, wherein the IL4/IL13 signature comprises one or more genes selected from the group consisting of:
a) FAM3C and CTNNB1; or
b) PHAX, LPP, PDE6A, MEFV, TVP23C, PLCXD1, PHACTR4, AP1S3, TRAF3IP2, PGAM5, SMYD4, UGGT1, DFFA, UBB, LOC284454, IFITM3, TUBB2A, IRF1, AXL, SPN, RNF41, ANGPTL4, TUBB2B, NMNAT1, TANGO2, HOOK3, S1PR2, HLA-H, LRTOMT, ZNF430, SCOC, ZNF793, CYP20A1, MDM2, KREMEN1, ZC3H12D, FBLIM1, LOC646214, PDDC1, HLA-E, KRT6C, HYDIN2, COX18, PPM1K, MBOAT1, LINC00294, ZNF526, P2RX5-TAX1BP3, GNE, NUDT19, KIF18B, PDE4C, LETM1, TLCD2, CHST6, METTL21A, IKZF3, POTEE, HLA-G, XPNPEP3, TMEM33, MOG, TSPYL1, RBMS2, TAPBP, ANKRD20A9P, STON2, CYP4V2, POTEM, OPA3, POLH, ZNF805, IFITM2, C17orf75, IRGQ, KCNQ1OT1, TAF8, UGDH-AS1, CCL5, NOL9, CHP1, ORAI2, CA5B, HLA-B, LYZ, TOR1AIP2, TRAPPC2, SGTB, ZNF264, XIAP, RAMP2-AS1, SCAI, ZBTB3, ZNF490, ORC4, DNAL1, FBXL18, CPT1A, TNFAIP8L1, LRRC57, RBM3, HLA-C, LOC148709, LYRM7, ATCAY, PRICKLE2-AS3, AGMAT, NF2, LOC100131257, ACTG1, RUNDC1, MAVS, RPL36A-HNRNPH2, PNPO, GDPD1, ILF3-AS1, LOC284023, PRR11, TMEM41B, ZBTB8A, TUBB4A, ZNF850, VHL, IVD, FOXK1, MDM4, CCDC142, TRPV1, UBC and SENP5; or
c) TM4SF1, IL1B; or
d) CD44, CTNNB1, POSTN and TNC; or
e) CD44, ANXA2, YBX1, EDN1, ARPC2, VAMP8, RPS18, EHF, HAS3, MBNL2, MTRNR2L9, CPXM2, NOS2, HMGB3, TNC, IFITM3, TSPAN3, BIK, CXADR, LGALS7, SERPINFI, TPM4, SERINC5, IGHG4, SERPINE2, NTRK2, PAPSS2, SDPR, ANXA3, RPL21, POSTN, CXCL2, MTRNR2L1, NDRG1, TPI1, SERPINB4, WIPI1, AL353147.1, CKB, GCLM, LRRC17, FAM11OC, RPL18A, CAV1, VAMP5, IGHA1, VSNL1, KCNE3, SPARCL1, ARID5B, ARL4C, MTRNR2L3, CXCL1, IGHG1, CNN3, CST1, MYO10, IGHG3, AAMDC, HSPH1, ENO1, VCL, SYNGR2, LAMTOR4, CCL26, TSLP, SERPINB2, TMEM123, TGIF1, IGHM, RPL7, CMYA5, SH3BGRL3, LOXL4, MAP3K8, ANO1, PGAM1, STOM, MYL9, PLS1, CTNNAL1, EFNA1, MTRNR2L8, RPL13A, PAWR, RPL41, LDLR, ITGB1, TCEB2, SERPINB10, NEDD9, SERPINB13, LDHA, DSE, DDX58, GPC3, SERF2, KLHL5, MRPS6, ADAM9, CDCP1, EGLN3, SMARCAD1, CTNNB1, MIF, PFN1, CTD-2228K2.5, CDH26, IGHA2, ST6GAL1, PLEKHA1, RPS25, GALNT7, CTD-2090113.1, SNRPG, RPL17, SRGN, ACTN1, MTRNR2L13, MORF4L1, RPSAP58, AREG, PLEKHA5, TMSB4X, IFITM1, SLC5A3, GEM, REXO2, KCNJ16, RPS4Y1, MMP10, NRG1, TBC1D9, LBH, FBXO32, TXNDC17, PHLDB2, SLC6A14, KRT6A, CP, TNFAIP3, CTD-2319112.1, SERTAD4-AS1, AMD1, FOSL1, LRRFIP1, IL6, UBBP4, BTG3, SLC2A1, IGJ, TMA7, SSR4, FGFBP1, HSD17B13, CCDC51, SMOC2, SHISA5, CTSC and XPR1; or
f) HLA-F, SOX4, MYL12B, BST2, SHC1, INHBA, IF1 6, SQSTM1, LYPLA1, RAP1B, MT1L, ANXA8L1, PLTP, NBPF10, MGST3, PLEKHB2, ICAM1, PHLDA1, PPP1CB, PAFAH1B2, SLC3A2, ANXA8L2, ISG20, GNG12, TNFRSF10B, GRN, DSG3, FN1, IF127, C6orf62, SERPINE1, BNIP3, HLA-L, AKIRIN1, SYPL1, EFNA1, CDKN1A, TUBA1A, HLA-A, CCND2, CACUL1, RPS26, B2M, FKBP9L, AHNAK2, IFITM1, TFPI2, RPS4Y1, SNAI2, KRT6A, SRP9, CTGF and PGK1; or
g) CDH3, MYO1B, HAS3, EHF, TNC, DKK3, SERPINB4, FABP5, TNFSF10, PTHLH, TBL1XR1, GJB2, CAV1, THBS1, TNS4, SHISA9, LOC100132247, KRT18, LITAF, HSPH1, CCL26, CSF3, KRT8, SPHK1, HK1, CDH6, SERPINB3, TP63, KRT23 and CTSC; or
h) ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4 and EGR1; or
i) ATF3, SYF2, ALDH3A1, DUSP1, SPAG4, NCOA7, CTGF, ZFP36, NR4A1, CYR61, HSPA1A, BTG2, MSMB, IL8, TFF3, SUOX, CLDN4, EGR1, GLUL, ANXA1, EGR3, JUN, ID1, LMNA, JUNB, KRT17, HSPA1B, PPP1R15A, TSC22D1, SERPINE1, ETS2, DNAJB1, MTSS1L, ERRFI1, KRT5, TIPARP, LGALS3, SERTADI, TSC22D3, PTP4A1, BPIFB1, MAFF, MUC5AC, FOSB, PMAIP1, HSPA8, ZFP36L2, IER2, NFKBIA, CDKN1A, BRD2, RPS16, CAPS, EZR, FAM107B, KRT19, F3, KLF4, RHOB, FOSL2, GPRC5A, SOCS3, TRIB1, MIR22HG, HSP90AA1, PRDX1, WEE1, FTL, SLC25A25, VMP1, DDIT3, RND3, ATP5G2, TACSTD2, HBEGF, CD55, EMP1, MCL1, CEBPD, FKBP5, YPEL5, ACTG1, C12orf57, KAL1, RN7SK, NR1D2, FXYD3, KLF5, FOS, ANKRD37, MIDN, PER2, STARD7, WFDC2, DUSP6, HSPB1, SEPW1, MYC, CXCL17, KLF9, SYT8, GPX4, MIR24-2, EPS8, AHNAK, JUP, ADH7, S100P, POLR2A, PPP1R10, GCNT1, GAPDH, IER3, HOOK1, HN1L, TOMM20, SCGB1A1, GSR, SERPINB5, S100A6, PEBP1, CDC42EP4, AQP3, ADRB2, DDIT4, PER1, ADM, IRF1, PRDX6, RPS14, TPT1, PLXNB1, BHLHE40, SQSTM1, SCP2, KRT15, SGK1, ACTB, NABP1, IGFBP3, PPP1R15B, SOD2, SOX9, CTSH, MXD1, THBS1, CIB1, RAP2B, NEDD4L, RAB34, PRDX5, ANAPC11, RAC1, ARF1, HS3ST1, MAOA, HLA-E, EID1, BAG5, SERPINH1, CITED2, AJUBA, CTSK, PAFAH1B2, GNB2L1, LMO7, PDPN, UBE3A, HNRNPAB, GSTP1, TM4SF1, PIM3, DLX5, C9orf3, HPGD, ANXA11, UBC, PLP2, EGR2, S100A10, LAMA5, ALOX15, MUC16, RPL4, TNS4, TUBB, RHOC, CYP2S1, PPAP2C, GPX1, QARS, CSNK1D, HINT1, PDLIM1, BLVRB, PSMB6, TSPO, CDCA7L, HNRNPF, RPS4X, EPB41L4A-AS1, IRF6, FSTL1, FTH1, UBL5, ADI1, PHLDA1, IFITM2, VAPA, PPP1CB, EIF4A3, UBB, SELM, NFKBIZ, UBR5, PTPN14, RAN, PPP2CA, CKS2, ELF3, EIF4A1, CSTB, TSPYL2, PLEC, UGDH, IBTK, TUBA1C, HLA-A, PHYHDI, RNA28S5, UBA52, RPL30, FLOT1, CHST9, TMEM14B, EIF1, PTHLH, STAT6, LRRC8A, MTRNR2L2, VPS37B, HN1, ELK4, NBN, MID1, RAB2A, CCND2, PSMB1, KDSR, SIK1, KDM2A, CDC42SE2, SLC35F5, CLTA, SF3B14, TUBAIB, C6orf48, SRSF3, RPS12, ARHGAP18, C16orf72, ALDOA, MRFAP1, LYPLAL1, JUND, TRAK1, MINK1, RPL11, HMGN2, LNX2, CFL1, TAGLN2, SPRY2, HLA-C, SDC4, EEF2, PCBP1, PER3, RPLP1, RPL8, REV1, S100A2, NDUFV1, EIF3K, MYADM, DDX3Y, KRT8, SLPI, CHD4, SEMA5A, TOR1AIP2, CHD2, GSN, GDI2, HLA-B, FLRT3, PERP, DNAJA1, OBSCN, TNFRSF1A, PSMB3, KLF6, WDR26, TFCP2L1, ZCRB1, LITAF, SNRNP70, EIF3I, SPINTi, FLOT2, SLC3A2, UGT2A2, ID2, TMEM59, HMGA1, CTNND1, FKBP9, TUBB4B, POLR2G, SLC9A3R1, SNHG5, HEBP2, RNF181, BCAM, SRI, ARHGDIA, HERPUDI, KLF10, MAT2A, LEPROT, SPCS1, SORL1, CCNL1, CYB5R3, USP22, MKNK2, SEC14L1, SEC61B, BTF3, FZD6, DAZAP2, SFN, MGST1, LAMB3, TNFRSF12A, OAT, CD99, CAV2, FLNA, STXBP3, RIPK4, SRSF7, EIF3D, TNPO1, MYL12B, GCLC, METTL7A, RPL28, F2R, RNA18S5, TMEM261, SOD1, YBX3, C1 lorf31, MYL6, ELOVL5, ATOX1, RPLP2, FBXL5, MAP3K13, MDK, TOB1, DLL1, HSP90AB1, SNCA, LAMA3, CSNK1A1, ATP1B3, RPS2, EIF4H, UBE2D3, CEBPB, TGFBR2, RPS3, GADD45B, GAS5, ZNHIT1, DDR1, JAG2, WNT4, NDFIP1, APH1A, OAZ1, HDAC7, ATP1A1, FAU, PPL, RAB11A, NCOA4, ATP50O, SF1, UQCRQ, NONO, FBXL3, ID3, DHCR24, IER5, OPTN, TMSB10, PPP1CA, ARPC5, RAB1A, TSKU, RPL19, BTG1, NACA, RAI14, CAPN1, 10-Sep, CDKN2AIP, PHB2, DDOST, S100A16, BLCAP, CSDE1, EDF1, CDK5RAP2, PRKAR1A, CNN2, FGFR3, ALDH3A2, TMX4, RPL7A, RPS27, C14orf166, RPL18, C7orf55-LUC7L2, GNG5, GHITM, IVNS1ABP, H3F3B, RBM3, RPL27, SMIM15, ARFGAP3, PPP4R1, NDUFB4, SSBP1, RPS21, DDB1, ALAS1, SERINC3, TMBIM6, AQP5, RPS5, EIF3F, 7-Mar, SDF4, RPL15, CDC42SE1, LGALS8, PAK1, NAMPT, EGFR, NDUFS1, GPR155, RAD23B, CD9, LIMK2, DLK2, CBR1, PPP2R5C, PRRC2C, RPS11, SH3GLB1, HNRNPAO, NUMA1, FGFR2, GABARAPL2, ETV3, H3F3C, SSH1, ATF4, SH3BGRL, ABHD2, ZFYVE21, RPS24, SYNCRIP, DDX17, RPS8, AFG3L2, RPL26, HNRNPA3, VPS26A, CHD8, RPS19, NCL, PDCD10, DST, POMP, APP, CLU, SRP9, SKP1, RPL41, RPL39, UQCRB, MTUS1, IFT81, USP8, RPL6, RNF19A, ADAM28, LSM3, ASPH, GOLGB1, HNRNPH1, CP, TPM3, EEF1D, PDIA4, PTMA, CMYA5, PGAP1, MPHOSPH6, MTRNR2L8, MALAT1, VMO1, ITGAV, PDIA3, TLK1, RPL21, MTRNR2L10, CXADR, SPCS2, HSPA5, POSTN, LARS, MORF4L1, ATPSI, TNC, AKAP9, TMSB4X, BPTF, SESN3, SYNE2, TTC3, C1R, RPL10, MT-ND6, SERPINB4, PHPT1, RPL34, IGFBP2, MTRNR2L1, SLC38A1, SMC6, ENAH, SON, HLA-DRA, YWHAE, HSP90B1, MTRNR2L7, MTRNR2L5, MTRNR2L11, MT-ND2, MTRNR2L3, CTC-338M12.5, MTRNR2L13, MMP10, MT-RNR1, IGHA1, IGJ, MT-ATP8, MT-ND1, MT-ND5, MT-RNR2, MT-CO2, MT-CYB, MT-ATP6, MT-ND4L, MT-CO3, IGKC, MT-ND4, and MT-CO1; or
j) SERPINB3, SERPINB4, FABP5, CTSC, GJB2, SPHK1, MBOAT1, ORAI2, KCNQ1OT1, PDDC1, CHP1, CCL5, CYP20A1, IGFBP7, LRTOMT, TOR1AIP2, XPNPEP3, LOXL4, TRAPPC2, ZNF490, GNE, LOC100131257, ZBTB8A, TAPBP, TNFAIP8L1, UGDH-AS1, CCL26, CDH6, IRF1, LOC646214, TNS4, PRICKLE2-AS3, KRT16, PRR11, RBMS2, LYZ, FAM3C, NOL9, POLH, DFFA, PPM1K, UBB, METTL21A, CAV1, IKZF3, PDE4C, SHISA9, AP1S3, HAS3, IRGQ, XIAP, FBLIM1, PHACTR4, SPN, C17orf75, HLA-B, HLA-C, KRT23, NMNAT1, HOOK3, KRT75, THBS1, CCDC142, HYDIN2, LETM1, CAV2, RUNDC1, TLCD2, PLCXD1, HLA-E, UGGT1, UBC, ZNF793, LOC643406, TNC, PNPO, FBXL18, EHF, ZNF264, SMYD4, LEPREL4, RPL36A-HNRNPH2, LRRC57, ANKRD20A9P, VHL, CDH3, ZNF850, MOG, TAF8, RNF41, GDPD1, KRT8, COTL1, PNPT1, ORC4, TRAF3IP2, NUDT19, RNF125, IVD, ODF2L, BTG1, OR7D2, MEFV, MDM4, CTNNB1, OPA3, LINC00294, ZNF526, CRX, CYP4V2, GLTP, KIF18B, SPREDI, MYO1B, ILF3-AS1, TANGO2, TMEM41B, SCAI, LYRM7, TRPV1, CYCS, TUBB2A, CA5B, ATCAY, ASTN2, SLC16A3, POTEE, PDE6A, P2RX5-TAX1BP3, TUBA4A, DDX51, CPT1A, TMEM212, KREMEN1, GPR82, S1PR2, SENP5, PHAX, TUBB4B, ZBTB3, TVP23C, PXMP4, WDR92, OPHN1, AP4S1, LOC284023, L2HGDH, TBC1D24, MTFMT, SGTB, PGAM5, SLFNL1-AS1, IFNLR1, GJC1, RAMP2-AS1, FKBP14, MDM2, KRT6C, AGMAT, CHST6, NWD1, MAVS, AKIP1, FOXK1, METTL2A, FDPSL2A, HLA-G, TFDP2, VSIG1, ZNF483, PTCHD4, SLC6A4, ZNF805, DNAL1, LOC284454, CD3EAP, PIGX, C10orf32, COX18, HLA-H, CPM, FBXO27, PALLD, LPP, NXN, NMT2, NPIPL3, PARD6B, VPS53, FUT1, GREB1, C21orf62, TINAGL1, WDR55, ZC3H12D, DTX3L, NF2, GATAD1, TRPM7, QPCTL, CFLAR, AXL, ALDH1A3, ARPP19, ZNF814, PSTPIP2, LOC90834, MREG, MPPE1, ZFP14, NT5DC3, SLC35F6, MAP1LC3C, PER2, UTP11L, TMEM136, KIN, NPHS1, GK5, PTHLH, LOH12CR2, GNL3L, MXRA7, TUBB4A, TSPYL1, POTEM, FBXL20, TBL1XR1, HP1BP3, TMEM33, PTRF, MYLK3, IFITM3, CABP4, LINC00338, ICA1L, MRI1, EMX2OS, IRAK4, IBA57, LOC728558, POU5F1, ZKSCAN3, PCDH11X, STON2, SLC50A1, LOC613037, AKAP5, ZNF430, ZNF587, EMP2, SLC5A5, STK4, RAC1, ARGFX, MCTS1, MAPK13, ULK2, CYB5R3, PEX13, LOC100132247, LOC100506190, MS4A10, PPP1CC, LIMS1, DCAF10, TBXA2R, ANAPC16, CD84, LOC284950, ACTG1, DLEU2, TRIM45, CCBE1, MED18, ME2, SLC28A2, ZNF738, DAND5, TTC39C, WHAMM, TMEM120B, FLVCR1, SMU1, CEP104, GTF2H3, FXN, IFITM2, RPS2, NME1-NME2, LOC727896, ZYG11B, LOC100507173, MTCH1, ATXN3, NLRP12, TUBB2B, ENPP1, MRPS16, SHOX, SLC35E3, GCLM, EDARADD, ESYT2, C12orf65, CNNM3, BRIP1, GOLGA3, FAM227A, TM7SF3, FCAR, DBT, CACNG8, ANPEP, LOC100287792, SLC12A6, LITAF, INMT, GTF2H2C, SIX4, CYP51A1, ALG1, SPIB, LRPAP1, DKK3, PRRG4, SNHG16, ARSA, RABL5, PACS2, DNAJC22, RELL1, LOC100289019, SCAMP4, KRT18, ZXDC, MOB3A, EXPH5, PCBD2, LOC286437, ANGPTL4, ABCC9, SNIP1, AFMID, Cllorf58, GEMIN8, LOC148709, PTGIS, ZNF785, RNF168, SHROOM1, ZSCAN29, CARD8, FAM122C, FAM73A, SDE2, HSPH1, NLRC3, HAUS3, IAPP, SPAST, PNMA2, LRRC58, LOC100506746, GFOD2, UBE2Q2P1, APOL1, LRP10, CORO2A, ZSCAN22, TUBB6, MIR143HG, HNF1A-AS1, ZNF865, SPATS2, SLC25A32, CCL22, LOC100505876, ADAMTS4, RPS6KA3, TRIM16L, PDLIM5, MPL, ENTPD4, ADAT1, SAR1B, UCKL1-AS1, CYP1A2, METTL2B, ASB6, CEACAM22P, ABL2, LRRC27, ENAH, TP63, ZFAND5, LOC100506688, AK3, PPIEL, OCLN, WAC, SPATA5, TNFSF10, SCOC, LRRN4CL, BHMT2, PTAFR, GSTM3, FKBP5, TNFAIP8L2-SCNM1, PDK3, ZNF714, CXorf56, TRMT2B, CBFA2T2, SLC35E2, MTDH, EEF2K, HAUS2, TPMT, CEP68, SLC4A8, TSIX, CXADR, KLRD1, TMEM165, IDS, SS18, EFNB1, APOBEC3F, ADRA1A, SLC25A15, CLCC1, CBX5, STYX, RBBP5, GNG4, RBM34, ZNF829, RBM3, JAK3, FZD3, ZYG11A, ARHGAP1, PLEKHG2, NUP43, CXorf38, TRIM58, ZNF818P, SKA1, MTPAP, Clorf174, MFSD11, MAPK1IP1L, GATM-AS1, DESI1, IVNS1ABP, GLT25D1, C4orf19, GLG1, RFT1, ZNF626, LPIN3, CSF3, GPR155, SSR1, FCF1, ZNF737, NDUFV3, ATP1A1, EIF2S3, ATP6V1G1, TADA3, CLSPN, TBC1D15, RAB27A, HK1, RTCA, ARNTL2, KCNA7, SMIM12 and ZSWIM1; or
k) IF127, HLA-A, HLA-B, HLA-C, HLA-H, IFITM3, RPS26, ICAM1, IFITM1, IF1 6, UBC, SERPINE1, TFPI2, EFNA1, TUBB2A, HLA-E, HLA-G, AHNAK2, TUBA1A, TUBB2B, POTEE, B2M, FN1, MBOAT1, TAPBP, KRT6C, RPS4Y1, TUBB4A, ORAI2, TOR1AIP2, SQSTM1, MT1L, RPL36A-HNRNPH2, TSPYL1, IFITM2, PDDC1, UBB, CHP1, CCL5, LYPLA1, NBPF10, ZBTB8A, PPP1CB, KCNQ1OT1, CYP20A1, ZNF490, IL1B, ANXA8L1, XPNPEP3, CTNNB1, LRTOMT, SHC1, STON2, LYZ, UGDH-AS1, POTEM, MDM2, NOL9, HLA-F, IKZF3, TNFAIP8L1, PPM1K, SLC3A2, AP1S3, PRICKLE2-AS3, DFFA, POLH, IRF1, PAFAH1B2, PLEKHB2, XIAP, UGGT1, CTGF, LOC100131257, TRAPPC2, PDE6A, FBLIM1, PDE4C, PRR11, PNPO, C17orf75, SRP9, RBMS2, CCDC142, AXL, ZNF793, KRT6A, TMEM41B, RAP1B, GNE, RBM3, FOXK1, INHBA, NMNAT1, IRGQ, TLCD2, BST2, NF2, TRAF3IP2, CA5B, SENP5, NUDT19, LINC00294, HOOK3, CYP4V2, LOC646214, CACUL1, PHACTR4, VHL, ZNF526, ZNF264, RUNDC1, HYDIN2, FBXL18, SPN, GDPD1, PHAX, TAF8, LRRC57, METTL21A, IVD, MGST3, SMYD4, ANXA8L2, PLTP, TVP23C, TM4SF1, ZBTB3, FKBP9L, MDM4, C6orf62, ILF3-AS1, TRPV1, LETM1, CHST6, TNFRSF10B, S1PR2, KIF18B, CPT1A, MOG, RNF41, SOX4, PLCXD1, PGAM5, LPP, GNG12, MEFV, ZNF850, OPA3, SCAI, TMEM33, LYRM7, SNAI2, SCOC, DSG3, LOC148709, SGTB, GRN, ATCAY, SYPL1, KREMEN1, BNIP3, ANGPTL4, RAMP2-AS1, MAVS, ZC3H12D, P2RX5-TAX1BP3, CDKN1A, ISG20, ANKRD20A9P, ZNF805, LOC284454, HLA-L, AGMAT, AKIRIN1, MYL12B, TANGO2, ACTG1, LOC284023, COX18, ORC4, PHLDA1, DNAL1, FAM3C, PGK1, ZNF430 and CCND2.

22. The method of anyone of the preceding claims wherein the modulating agent is an antibody, or antigen binding fragment, an aptamer, affimer, non-immunoglobulin scaffold, small molecule, or genetic modifying agent.

23. An isolated barrier tissue cell characterized by expression of one or more markers from one of clusters 0 to 21 in Table 1.

24. The isolated barrier tissue cell of claim 23, wherein the isolated barrier tissue cell is a basal cell, the basal cell characterized by expression of one or more markers selected from:
a. clusters 2, 8, and 12 of Table 1;
b. selected from the group consisting of; S100A2, KRT5, KRT15, POSTN, MMP10, PERP, AQP3, EGR1, CD9, MIR205HG, F3, FOS, TACSTD2, KRT17, ALOX15, ETS2, JUNB, KRT19, DST, TNC, TSC22D1, ID1, TP63, LAMB3, CLDN1, IL33, ALDH3A1, SERPINFI, NCOA7, BTF3, FXYD3, PRSS23, ALDH3A2, SFN, CYR61, ATF3, SGK1, RPL10A;
c. selected from the group consisting of; POSTN, S100A2, KRT5, KRT15, JUNB, MMP10, EGR1, MIR205HG, KRT17, TNC, RPL3, ETS2, DST, SERPINF1, TP63, RPL13A, RPS25, EIF1, IFITM3, IL33, LAMB3, RPL10A, RPL4, BTF3, RPS9; or
d. selected from the group consisting of; KRT5, TSC22D1, KRT15, S100A2, DST, ALDH3A2, MIR205HG, CLDN1, TP63, KRT17, RASSF6, CYR61, ETS2, ADH7, MPZL2, BCAM, SLC6A6, PDPN, TNC, SFN, LAMB3, NTRK1, NTRK2, and SPINK5.

25. The isolated barrier tissue cell of claim 23, wherein the isolated barrier tissue cell is a fibroblast cell, the fibroblast cell characterized by expression of one or more markers selected from;
a. clusters 5 and 14 of Table 1; or
b. selected from the group consisting of; DCN, COL1A2, LUM, COL3A1, MGP, LGALS1, CALD1, IGFBP7, FBLN1, CPE, SPAR, VIM, POSTN, IFITM3, SFRP1, SFRP2, CIS, COL1A1, SERPINGI, AEBP1, PCOLCE, TAGLN, C1R, SEPP1, PPAP2B, CRABP2, TPM2, IGFBP6, THY1, CDH11, CXCL14, FGF7, SELM, TMSB4X, RARRES2, VCAN, PRRX1, CLDN11, TPM1, NNMT, IGFBP4, BGN, LAPTM4A, PDGFRA, COL6A2, MXRA8, LIMA1, S100A6, APOD, FSTL1, LAMP5, NBL1, THBS1, EID1, IL6ST, KCNE4, IGF2, COL6A1, CCL2, MFAP4, COL15A1, ITGBL1, COL8A1, GLIPR1, TIMP3, RGS5, MYL9, ITGB1, APP, RARRES1, SERPINF 1, TGM2, and CLU.

26. The isolated barrier tissue cell of claim 23, wherein the isolated barrier tissue cell is a myeloid cell, the myeloid cell characterized by expression of one or more markers selected from;
a. cluster 11 of Table 1; or
b. selected from the group consisting of; HLA-DRA, CD74, HLA-DRB1, SRGN, HLA-DPB1, HLA-DPA1, TMSB4X, FTH1, AIF1, TMSB10, TYROBP, FTL, CST3, GPR183, HLA-DQA1, IF1 30, HLA-DRB5, FGL2, ACTB, CTSS, IL8, PLAUR, LAPTM5, HLA-DQB1, PSAP, MS4A6A, FCER1G, NFKBIA, COTL1, DUSP2, HLA-DMA, IL1B, CPVL, MNDA, NAMPT, VIM, RGS2, CD83, PTPRC, ITGB2, SH3BGRL3, PLEK, LST1, TNFAIP3, OAZ1, BCL2A1, HLA-DMB, CLEC10A, LCP1, GPX1, F13A1, NPC2, TPM3, AMICA1, PFN1, HLA-B, CCL3, SAMSN1, ZNF331, ARPC1B, CYBB, NR4A2, PPP1R15A, ARPC2, RGS1, ARPC5, ARHGDIB, HLA-E, CTSH, CD68, CTSB, CD14, ACTR2, IGSF6, ARPC3, PTPRE, CFL1, ATP5E, CD52, CLEC7A, GRB2, MS4A7, SAMHD1, C5AR1, S100A4, CXCL2, CTSC, AREG, SOD2, S100A9, FCGRT, PABPC1.

27. The isolated barrier tissue cell of claim 23, wherein the isolated barrier tissue cell is an apical cell, the apical cell characterized by expression of one or more markers selected from;
a. clusters 0, 1, and 4 of Table 1; or
b. selected from the group consisting of; SERPINB3, KRT19, S100A6, AGR2, ANXA, CLDN4, ELF3, SLPI, WFDC2, PRSS23, KRT8, TACSTD2, HSPB1, VMO1, SAT1, KRT18, TSPAN1, GSTP1, SERPINB4, AQP3, UGT2A2, EPAS1, ALDH1A1, LGALS3, ANXA2, PERP, EZR, CD9, TXN, ATP1B1, F3, MGST1, ALCAM, CXCL17, MT1X, FXYD3, PRDX1, S1OOP, GABRP, NTS, CSTB, ALOX15, CTG1, KRT7, CP, HES1, S100A11, DUSP1, CTSB, CLDN7, ATF3, ADAM28, KLF5, TNFSF10, SPINT2, CST1, and CD55.

28. The isolated barrier tissue cell of claim 23, wherein the isolated barrier tissue cell is a glandular epithelium cell, the glandular epithelium cell characterized by expression of one or more markers selected from;
a. clusters 3 and 13 of Table 1;
b. selected from the group consisting of; LYZ, SLPI, AZGP1, PIGR, BPIFB1, LTF, ZG16B, STATH, TCN1, BPIFA1, PIP, C6orf58, DMBT1, RP11-1143G9.4, ODAM, XBP1, CXCL17, RNASE1, WFDC2, CCL28, NUCB2, NDRG2, SLC12A2, SCGB3A1, CA2, EHF, FAM3D, LRRC26, AQP5, PHLDA1, TMED3, PART1, CST3, PPP1R1B, MSMB, CLDN10, KIAA1324, FDCSP, P4HB, PRR4, HP, and MT-ND3; or
c. selected from the group consisting of; LYZ, AZGP1, LTF, ZG16B, STATH, TCN1, BPIFB1, PIGR, SLPI, BPIFA1, PIP, C6orf58, DMBT1, RP11-1143G9.4, ODAM, XBP1, RNASE1, NUCB2, CCL28, SEC11C, SSR4, SCGB3A1, NDRG2, CA2, PHLDA1, CST3, CXCL17, LRRC26, SLC12A2, PPP1R1B, PART1, TMED3, FDCSP, FAM3D, PRR4, and HP; or
d. selected from the group consisting of; LTF, ZG16B, STATH, AZGP1, TCN1, C6orf58, DMBT1, PIP, RP11-1143G9.4, ODAM, FDCSP, LPO, MUC5B, SCGB3A1, CCL28, CA2, NDRG2, SLC12A2, LRRC26, HP, PART1, PPP1R1B, CLDN10, and S100A1.

29. The isolated barrier tissue cell of claim 23, wherein the isolated barrier tissue cell is a differentiating/secretory cell, the differentiating/secretory cell characterized by expression of one or more markers selected from;
a. Table 15; or
b. the group consisting of; VMO1, LYPD2, PSCA, SCGB1A1, S1OOP, MSMB, ALCAM, KRT7, KRT8, MGST1, MUC5AC, SPDEF, LCN2, CLDN7, PI3, SLC31A1, GABRP, TMEM213, STEAP4, GDF15, S100A14, MSLN, SORD, NTS, MUC16, ST6GALNAC1, SLC9A3R1, MUC1, CST1, CST4, IGFBP3, SLC6A14, CST2, CDH26, EGLN3, and POR.

30. The isolated barrier tissue cell of claim 23, wherein the isolated barrier tissue cell is a ciliated cell, the ciliated cell characterized by expression of one or more markers selected from;
a. cluster 16 of Table 1;
b. selected from the group consisting of; CAPS, C9orf24, TSPAN1, PIFO, TPPP3, C20orf85, SNTN, FAM183A, TUBB4B, TUBA1A, GSTA1, Cllorf88, RSPH1, PRDX5, OMG, AGR3, CAPSL, CIB1, CCDC170, DYNLT1, HSP90AA1, IFT57, DNAH5, DYNLL1, EZR, TMEM190, Clorf194, NUCB2, CALM1, ATPIF1, MORN2, RP11-356K23.1, PSENEN, SPA17, C9orf116, ZMYND10, ROPN1L, CETN2, LRRIQ1, DNAH12, C5orf49, PLAC8. TMC5, GSTP1, CCDC146, Clorf173, CALM2, CYP4B1, CHST9, TCTEX1D4, ARL3, CD59, FAM216B, SPAG6, FAM154B, FAM81B, FAM229B, SMIM22, EFCAB1, NQO1, ABCA13, IK, ARMC3, FOXJ1, CDHR3, SCGB2A1, IQCG, PRDX1, RRAD, ANXA1, TSPAN19, PCM1, FAM92B, DYDC2, RSPH4A, SLC44A4, UFC1, DNALI1, CKB, NME5, TEKT1, ODF3B, C9orf135, ALDH1A1, LGALS3, WDR78, ODF2L, HSPH1, ALDH3B1, TSPAN6, LRRC23, WDR52, CTSS, MS4A8, SPAG16, ENKUR, EFHC1, PSCA, NUDC, HMGN3, ZBBX, MLF1, KIF21A, RSPH9, Clorf192, CCDC11, CCDC113, AK7, AKAP9, LDLRAD1, WDR54, KIF9, EFCAB10, WDR96, C12orf75, DYNLRB2, HSPB11, FXYD3, TSTD1, HSBP1, AKAP14, WDR86-AS1, C10orf107, Cllorf70, CES1, MNS1, SPEF2, SPATA18, CCDC17, NPHP1, DPY30, TAX1BP1, TCTEX1D2, ARHGAP18, PPIL6, C14orf142, C21orf59, GSTA2, CCDC19, TMEM231, C6orf118, STOML3, FANK1, SEPW1, SPAG1, ALCAM, ANXA2, CSPP1, DHRS9, MRPS31, TSPAN3, CYSTM1, RP11-867G2.2, SRI, NEK10, ANKUB1, SYNE1, DPCD, CATSPERD, CCDC39, NWD1, MORN5, CD164, CLDN7, S100A6, SAMHD1, DNPH1, SPAG17, RP11-275114.4, B9D1, WDR66, LRRC46, MAP3K19, LRRC48, EFCAB2, AGR2, LINC0094, DNAAF1, PROM1, DNAJA4, CDS1, C9orf117, FHAD1, DNAH3, OSCP1, FAM174A, H2AFJ, WFDC2, PIH1D2, RABL5, PERP, IF127, CCDC173, IGFBP2, SAT1, DTHD1, CCDC42B, DNAH9, CCDC176, LZTFL1, SOD1, CLU, CCDC65, Cllorf74, CTGF, DRC1, CASC1, DSTN, TRAF3IP1, CCDC104, YWHAE, COX6A1, TMBIM6, IFT172, SLC27A2, LRP11, S100A11, ALOX15, IFT43, TXN, STK33, ARMC4, DZIP3, RAB11FIP1, UBXN10, IFT81, IGFBP7, TTC18, CYB5A, CAST, TMEM59, ELF3, UBB, DNAH11, C7orf57, PTGES3, TTC29, PPP1R42, CLDN3, MUC16, TUSC3, TCTN1, POLR2I, CCDC78, RUVBL2, TNFAIP8L1, CC2D2A, GDF15, TAGLN2, CDHR4, DNAL1, ECT2L, RUVBL1, SYAP1, METTL7A, DNAH7, IQCD, NDUFB1, UBL5, RP4-666F24.3, C9orf9, C21orf58, ANKRD66, EPCAM, PCDP1, CMPK1, TMEM14B, MORF4L2, and MT-RNR1; or
c. from the group consisting of; CAPS, C9orf24, PIFO, TPPP3, C20orf85, SNTN, FAM183A, Cllorf88, RSPH1, CAPSL, TMEM190, Clorf194, MORN2, RP11-356K23.1, SPA17, C9orf116, ZMYND10, ROPN1L, LRRIQ1, DNAH12, C5orf49, CCDC146, Clorf173, TCTEX1D4, FAM216B, SPAG6, FAM154B, FAM81B, FAM229B, SMIM22, EFCAB1, ARMC3, FOXJ1, CDHR3, IQCG, RRAD, TSPAN19, FAM92B, DYDC2, RSPH4A, DNALI1, NME5, TEKT1, ODF3B, and C9orf135.

31. The isolated barrier tissue cell of claim 23, wherein the isolated barrier tissue cell is a plasma cell, the plasma cell characterized by expression of one or more markers selected from:
a. clusters 7, 10, 15, and 17 of Table 1;
b. or selected from the group consisting of; IGJ, SSR4, MZB1, IGHA1, SEC11C, HSP90B1, ENAM, IGHG1, IGHA2, IGHG4, IGHG3, HERPUD1, DERL3, PRDX4, IGHG2, FKBP11, IGKC, AC096579.7, SPCS3, RGS1, TSC22D3, SLAMF7, FAM46C, SSR3, PIM2, RNA28S5, CD79A, XBP1, ITM2C, IGLC7, SEL1L, IGLL1, FCRL5, PRDM1, TRAM1, UBE2J1, RRBP1, SUB1, SPCS1, CCND2, IGLC3, ERLEC1, FKBP2, SPCS2, SELK, IGKV3-20, ISG20, CYBA, and C19orf10.

32. The isolated barrier tissue cell of claim 23, wherein the isolated barrier tissue cell is a endothelial cell, the endothelial cell characterized by expression of one or more markers selected from:
a. cluster 6 from Table 1; or
b. from the group consisting of; SPARCL1, VIM, HLA-E, GNG11, A2M, TMSB10, IF127, IGFBP7, IFITM3, CD74, CLDN5, ELTD1, TMSB4X, DARC, EMCN, TM4SF1, SPARC, PTRF, VWF, GIMAP7, IFITM2, PLVAP, RPS23, RPL3, ECSCR, RPL32, EMP1, HLA-DRB1, RAMP2, CALCRL, PTMA, HLA-DRA, RPL31, ESAM, ID3, APOLDI, FKBP1A, ADAMTS1, ADIRF, RPS27A, RAMP3, RPS15A, EGFL7, HLA-B, RPS13, IL33, RPS3, LIFR, CAV1, NPDC1, CD34, AC011526.1, RPS9, NOSTRIN, RPL24, IL6ST, CYYR1, CRIP2, RDX, RPL5, JAM2, TGFBR2, STOM, TPM3, TXNIP, HLA-DPA1, RPL26, TSPAN7, ENG, SPRY1, EEF1A1, PALMD, SPTBN1, RPL9, CD93, ELK3, SOCS3, MYL12A, RPL19, SELE, KCTD12, RPL12, HLA-DRB5, and RPL10

33. The isolated barrier tissue cell of claim 23, wherein the isolated barrier tissue cell is a T cell, the T cell characterized by expression of one or more markers selected from;
a. cluster 9 of Table 1;
b. the group consisting of; TMSB4X, RPS29, RPS27A, CD52, RPS15A, RPS27, CXCR4, RPS25, SRGN, PTPRC, TRBC2, RPL13A, RPS20, HLA-B, IL32, CCL5, RPL32, RPL23A, CD2, RPS3, RPL37, CD3D, IL7R, HLA-E, PFN1, ARHGDIB, RPL39, TSC22D3, PTPRCAP, CD69, RPL14, RGS1, RPL10, HLA-C, HLA-A, S100A4, TNFAIP3, LCP1, UBA52, ETS1, KLRB1, and GZMA.

34. The isolated barrier tissue cell of claim 23, wherein the isolated barrier tissue cell is a mast cell, the mast cell characterized by expression of one or more markers selected from;
a. TPSAB1, CPA3, CD69, SRGN, HPGD, RGS1, HPGDS, SLC18A2, SAMSN1, KIT, NFKBIZ, HDC, CTSG, ACSL4, FTH1, LAPTM5, TMSB4X, TNFAIP3, TPSD1, CD52, PTGS2, GATA2, NFKBIA, PPP1R15A, IL1RL1, VIM, FTL, DUSP6, AHR, MS4A2, CD63, NR4A2, Clorf186, VWA5A, CLU, AREG, SELK, RGS2, CCL4, ANXA1, ALOX5AP, GPR65, TYROBP, GLUL, RGS13, S100A4, FOSB, CAPG, UBB, TSC22D3, FCER1G, PTMA, GCSAML, ALAS1, CTSD, NR4A1, KLF6, RAC2, BTG, ARHGDIB, RP11-354E11.2

35. A method of modulating barrier cell proliferation, differentiation, maintenance and/or function, comprising: contacting a barrier cell or population of barrier cells with a barrier cell modulating agent in an amount sufficient to modify differentiation, maintenance, and/or function of the barrier cell or population of barrier cells as compared to differentiation, maintenance, and/or function of the barrier cell population or population of barrier cells in the absence of the barrier cell modulating agent.

36. The method of claim 35, wherein contacting the barrier cell or population of barrier cells with the barrier cell modulating agent rebalances barrier cell type diversity relevant to normal and/or non-diseased barrier cell tissue

37. The method of claim 35, wherein modulating barrier cell proliferation, differentiation, maintenance and/or function is used to treat or prevent type 2 inflammatory responses in barrier tissues.

38. The method of claim 35, wherein the barrier cell modulating agent modulates one or more of any of the targets: KRT5, KRT8, FOXJ1, LTF, DARC, COL1A2, CD79A, HLA-DRA, TRBC2, TPSAB1, CCL26, CCL11, CCL24, CXCL17, CCL28, HPGDS, PTGS2. ALOX5, ALOX15, IL-18, IL-1B, TP63, SCGB1A1, PIFO, MUC5B, MSMB, SCGB3A1, PSCA, LYPD2, CST4, CST1, TFF3, POSTN, PTHLH, SERPINB2, HS3ST1, CDH26, MMP10, SPINK5, ALDH3A1, CLCA4, GLUL, WNT, Notch, ITGA8, FN1, EPAS1, NTRK2, FGFBP1, ETS2, CSRP2, SULTIE1, FAM107A, PTN, ID2, EGFR, PAPPA, NNMT, COL1A1, LAMB1, ABCA5, LIMA1, TRAPPC3L, SMOC1, COL16A1, DCBLD2, RGS4, SLC39A6, EFEMP1, OXTR, PLOD2, LINC00152, TNFRSF12A, SERPINE1, CALU, TPM4, MPP4, RHOJ, FAM65C, ABCA8, KANK4, FGF10, FGFBP2, ABCA3, HUNK, PRB4, TENM1, CLMN, RIC3, BPIFB2, CCBE1, ATP1A2, CNTN3, COL11A1, PNISR, CCL2, CXCL12, CCL5, CCL13, IL-6, IL-10, IGFBP3, STEAP4, or EGLN3.

39. The method of claim 38, wherein one or more modulating agents modulate at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104 or more of the targets, or at least (or equal to) 1-5, 5-10, 10-20, 20-50, 50-110 of the targets.

40. The method of claim 35, wherein the barrier cell modulating agent modulates one or more of any of the targets: TBXAS1, MUC5AC, FOXA3, SPDEF, IGFB3, ELGN3, LGR6, CD44, p63, FOXA1, Bach2, Sox, IF144L, AP-1, STAT, CCL17, CCL18, PTGES, PTGDS, TGFB2, TNF, S100A8, or S100A9.

41. The method of claim 40, wherein one or more modulating agents modulate 2, 3, 4, 5, 6, 7, 8, 9, 10, 11, 12, 13, 14, 15, 16, 17, 18, 19, 20, 21, 22, or 23 of the targets.

42. The method of claim 35, wherein the barrier cell modulating agent modulates one or more of the genes in Table 1, Table 2, Table 3, or a combination thereof

43. The method of any one of claims 35 to 37, wherein the barrier cell modulating agent modulates one or more of any of CCL26, CCL11, CCL24, CXCL17, CCL28.

44. The method of claim 43, wherein the barrier cell modulating agent inhibits expression of
a. CCL26 in the basal cells of claim 24 and/or the fibroblasts of claim 25;
b. CCL11 in the fibroblasts of claim 25;
c. CCL24 in the myeloid cells of claim 26;
d. CXCL17 in the apical cells of claim 27 and/or the glandular epithelium cells of claim 6;
e. CCL28 in the glandular epithelium of claim 28; or
f. a combination thereof.

45. The method of claim 44, wherein the barrier cell modulating agent modulates one or more of HPGDS, PTGS2, ALOX5, and TBXAS.

46. The method of claim 45, wherein the barrier cell modulating agent inhibits expression of;
a. HPGDS, PTGS2, and/or ALOX5 expression in the mast cells of claim 34; or
b. TBXAS expression in the myeloid cells of claim 26.

47. The method of any one of claims 13 to 37, wherein the barrier cell modulating agent inhibits IL-33 and/or TSLP expression or prevents IL-33 or TSLP from binding to a corresponding receptor.

48. The method of claim 47, wherein the barrier cell modulating agent inhibits;
a. IL-33 expression in the basal cells of claim 24, the apical cells of claim 27, and/or the ciliated cells of claim 30; or
b. TSLP expression in the basal cells of claim 24.

49. The method of any one of claims 35 to 39, wherein the barrier cell modulating agent inhibits IL4, IL5, IL13, HPGDS, and/or AREG expression, or prevents IL4, 115, IL13, HPGDS, and/or AREG from binding to a corresponding receptor

50. The method of claim 49, wherein the barrier cell modulating agent inhibits;
a. IL4, IL15, IL13, and/or HPGDS expression in the T cells of claim 33;
b. IL5 and IL13 expression in the mast cells of claim 34;
c. AREG expression in the mast cells of claim 34 and/or the myeloid cells of claim 4; or
d. a combination thereof.

51. The method of any one of claims 35 to 37, wherein the barrier cell modulating agent either;
a. inhibits expression of one or more of CST4, CST1, IGFBP3, TFF3, and ELGN3 in the glandular epithelium cells of claim 6 the differentiating/secretory cells of claim 7, or the apical cells of claim 27; or
b. induces increased expression of MSMB, SCGB1A1, STEAP4, PSCA, LYPDL in the glandular epithelium cells of claim 6 the differentiating/secretory cells of claim 7, or the apical cells of claim 27; or
c. a combination thereof

52. The method of any one of claims 35 to 37, wherein the barrier cell modulating agent increases production of secreted mucins in the barrier tissue.

53. The method of claim 52, wherein the secreted mucins are MUC5B and MUC5AC.

54. The method of claim 52 or 53, wherein the barrier cell modulating agent increases;
a. production of MUC5B and MUC5AC+ by increasing expression of SPDEF; or
b. production of MUC5AC+ by increasing expression of SCGB1A1 and/or FOXA3.
The method of any one of claims 35 to 39, wherein the barrier cell modulating agent induces differentiation of basal cells.

55. The method of claim 0, wherein the barrier cell modulating agent inhibits expression of one or more of DLK2, DLL1, JAG2, DKK3, POSTN, FN1, and TNC in the basal cells of claim 24.

56. The method of claim 0, wherein the barrier cell modulating agent either;
a. inhibits expression of one or more AP-1 transcription factor family members, including JUN, FOXA1, BACH2, and p63; or
b. increases expression of one or more SOX/STAT/MEF2 transcription factor family members; or
c. a combination thereof

57. The method of any one of claims 35 to 56, wherein the barrier cell modulating agent induces differentiation of basal cells.

58. A method of modulating basal cell proliferation, differentiation, maintenance and/or function comprising administering, to the basal cell, an effective amount of one or more modulating agents able to interact with one or more genes or gene expression products in Table 4.

59. The method of claim 58, wherein the one or more genes or gene expression products is one or more of POSTN, PTHLH, ALOX15, SERPINB2, HS3ST1, CDH26, MMP10, CCL26, SPINK5, ALDH3A1, CLCA4, and/or GLUL.

60. The method of claim 58 wherein one or more of POSTN, PTHLH, ALOX15, SERPINB2, HS3ST1, CDH26, MMP10, or CCL26, is decreased or one or more of SPINK5, ALDH3A1, CLCA4, or GLUL is increased, relative to prior to administration of the effective amount of one or more modulating agents.

61. A method of modulating basal cell proliferation, differentiation, maintenance and/or function comprising administering, to the basal cell, an effective amount of one or more modulating agents able to interact with one or more of KRT5, IL-33, TSLP, and/or TP63.

62. The method of claim 61, wherein one or more of KRT5, IL-33, TSLP, and/or TP63 is increased relative to prior to administration of the effective amounts of one or more modulating agents.

63. A method of modulating endothelial cell proliferation, differentiation, maintenance and/or function comprising administering, to the endothelial cell, an effective amount of one or more modulating agents able to interact with one or more genes or gene expression products in Table 5.

64. The method of claim 16, wherein the one or more genes or gene expression products is MSMB.

65. The method of claim 64, wherein MSMB is increased relative to prior to administration of the effective amounts of one or more modulating agents.

66. A method of modulating endothelial cell proliferation, differentiation, maintenance and/or function comprising administering, to the endothelial cell, an effective amount of one or more modulating agents able to interact with one or more genes or gene expression products, wherein the one or more genes or gene expression products is one or more of IL-33, IL-18, SCGB1A1, SCGB3A1, PSCA, or LYPD2.

67. The method of claim 66, wherein one or more of IL-33 or IL-18 is decreased and/or one or more of, SCGB1A1, SCGB3A1, PSCA, and/or LYPD2 is increased relative to prior to administration of the effective amounts of one or more modulating agents.

68. A method of modulating endothelial cell proliferation, differentiation, maintenance and/or function comprising administering, to the endothelial cell, an effective amount of one or more modulating agents able to interact with one or more genes or gene expression products, wherein the one or more genes or gene expression products is DARC.

69. The method of claim 68, wherein DARC is increased relative to prior administration of the effective amounts of one or more modulating agents.

70. A method of modulating fibroblast cell proliferation, differentiation, maintenance and/or function in barrier tissues comprising administering, to the fibroblast cell, an effective amount of one or more modulating agents able to interact with one or more genes or gene expression products in Table 6.

71. The method of claim 70, wherein the one or more genes or gene expression products is one or more of CCL26 and/or CCL11.

72. The method of claim 71, wherein one or more of CCL26 or CCL11 is decreased relative to prior to administration of the effective amounts of one or more modulating agents.

73. A method of modulating fibroblast cell proliferation, differentiation, maintenance and/or function in barrier tissues comprising administering, to the fibroblast cell, an effective amount of one or more modulating agents able to interact with one or more genes or gene expression products wherein the one or more genes or gene expression products is one or more of COL1A2, and/or ITGA8.

74. The method of claim 73 wherein one or more of COL1A2, and/or ITGA8 is increased/decreased relative to prior to administration of the effective amounts of one or more modulating agents.

75. A method of modulating macrophage cell proliferation, differentiation, maintenance and/or function in barrier tissues comprising administering, to the macrophage cell, an effective amount of one or more modulating agents able to interact with one or more genes or gene expression targets in Table 7.

76. A method of modulating mast cell proliferation differentiation, maintenance and/or function in barrier tissues comprising administering, to the macrophage cell, an effective amount of one or more modulating agents able to interact with one or more genes or gene expression targets in Table 8.

77. The method of claim 76, wherein the one or more genes or gene expression products is one or more of HPGDS, PTGS2, or AREG.

78. The method of claim 77, wherein one or more of HPGDS, PTGS2, or AREG is decreased relative to prior to administration of the effective amounts of one or more modulating agents.

79. A method of modulating mast cell proliferation differentiation, maintenance and/or function in barrier tissues comprising administering, to the mast cell, an effective amount of one or more modulating agents able to interact with one or more genes or gene expression targets, wherein the one or more genes or gene expression products is TPSAB1, IL-5, ALOX5 and/or IL-13.

80. The method of claim 79, wherein one or more of TPSAB1, IL-5, ALOX5, and/or IL-13 is increased/decreased relative to prior to administration of the effective amounts of one or more modulating agents.

81. A method of modulating plasma cell proliferation, differentiation, maintenance and/or function in barrier tissues comprising modulating one or more genes or gene expression targets in Table 9.

82. A method of modulating T cell proliferation, differentiation, maintenance and/or function in barrier tissues comprising modulating one or more genes or gene expression targets in Table 10.

83. The method according to any one of the preceding claims, wherein the barrier modulating agent comprises a CRISPR system, a zinc finger system, TALE, TALEN, therapeutic antibody, bi-specific antibody, antibody fragment, antibody-like protein scaffold, aptamer, RNAi or small molecule.

84. A method of identifying a basal cell comprising detecting one or more genes or gene expression products of claim 2.

85. The method of claim 84, wherein the gene or gene expression products are one or more of POSTN, PTHLH, ALOX15, SERPINB2, HS3ST1, CDH26, MMP10, CCL26, SPINK5, ALDH3A1, CLCA4, or GLUL.

86. The method of claim 85, wherein a basal cell in diseased tissue is identified by detecting an increase in one or more of POSTN, PTHLH, ALOX15, SERPINB2, HS3ST1, CDH26, MMP10, or CCL26, and/or a decrease in one or more of SPINK5, ALDH3A1, CLCA4, or GLUL compared to a basal cell from non-disease tissue.

87. A method of identifying a basal cell comprising detecting one or more genes or gene expression products, wherein the one or more genes or gene expression products is one or more of KRT5, IL-33, TSLP, and/or TP63.

88. The method of claim 87, wherein one or more of KRT5, IL-33, TSLP, and/or TP63 is increased compared to a basal cell from non-disease tissue.

89. A method of identifying an endothelial cell comprising detecting one or more genes or gene expression products in Table 5.

90. The method of claim 89, wherein the one or more genes or gene expression products is MSMB.

91. The method of claim 90, wherein MSMB is decreased compared to an endothelial cell from non-disease tissue.

92. A method of identifying an endothelial cell comprising detecting one or more genes or gene expression products, wherein the one or more genes or gene expression products is one or more of IL-33, IL-18, SCGB1A1, SCGB3A1, PSCA, or LYPD2.

93. The method of claim 92, wherein an endothelial cell from diseased tissue is identified by detecting an increase in one or more of IL-33 or IL-18, and/or a decrease in one or more of SCGB1A1, SCGB3A1, PSCA, or LYPD2 compared to an endothelial cell from non-disease tissue.

94. A method of identifying an endothelial cell comprising detecting one or more genes or gene expression products, wherein the one or more genes or gene expression products is DARC.

95. The method of claim 94, wherein DARC is increased/decreased compared to an endothelial cell from non-disease tissue.

96. A method of identifying a fibroblast cell comprising detecting one or more genes or gene expression products in Table 6.

97. The method of claim 96, wherein the one or more genes or gene expression products is one or more of CCL26 and/or CCL1 1.

98. The method of claim 97, wherein a fibroblast from disease tissue is identified by detecting an increase in one or more of CCL26 and/or CCL11 compared to a fibroblast from non-disease tissue.

99. A method of identifying a fibroblast cell comprising detecting one or more genes or gene expression products, wherein the one or more genes or gene expression products is one or more of COLIA2, and/or ITGA8.

100. A method of identifying a macrophage cell comprising detecting one or more target genes or target gene expression products in Table 7.

101. A method of identifying a mast cell comprising detecting one or more target genes or target gene expression products in Table 8.

102. The method of claim 101, wherein the target gene or target gene expression products are one or more of HPGDS, PTGS2, AREG.

103. The method of claim 102, wherein a mast cell from disease tissue is identified by detecting an increase in one or more of HPGDS, PTGS2, or AREG compared to a mast cell from non-disease tissue.

104. A method of identifying a mast cell comprising detecting one or more genes or gene expression targets, wherein the one or more genes or gene expression targets is TPSAB1, IL-5, ALOX5 and/or IL-13.

105. The method of claim 104, wherein one or more of TPSAB1, IL-5, ALOX5 and/or IL-13 is increased/decreased compared to a mast cell from non-disease tissue.

106. A method of identifying a plasma cell comprising detecting one or more genes or gene expression targets in Table 9.

107. A method of identifying a T cell comprising detecting one or more genes or gene expression targets in Table 10.

108. A method of treating, preventing or ameliorating chronic type 2 inflammation in barrier tissue comprising administering a modulating agent to a subject in need thereof, wherein the modulating agent restores cell diversity in epithelial tissue.

109. The method of claim 109, wherein restoring cell diversity in epithelial tissue comprises inducing basal cell differentiation.

110. The method of claim 108 or 109, wherein the modulating agent inhibits IL4, IL5 IL13, and/or NGF.

111. The method of claim 108 or 109, wherein the modulating agent inhibits WNT.

112. The method of claim 108 or 109, wherein the modulating agent increases expression of Notch, DLL1, DLL2, or a combination thereof.

113. A method of detecting a type 2 inflammation induced tissue disease comprising:
producing a single cell transcriptome/proteome for one or more cells from a first population of cells in a first sample;
analyzing the transcriptome/proteome from the first sample to determine sub-populations of cells in the first sample;
comparing the transcriptome/proteome of the one or more cells from the first population of cells with a transcriptome/proteome from a second population of cells; and
determining a difference in the expression of any one of the following genes:
i) one or more of IL4, IL5, or IL13;
ii) Wnt or Notch;
iii) POSTN, PTHLH, ALOX15, SERPINB2, HS3ST1, CDH26, MMP10, CCL26, SPINK5, ALDH3A1, CLCA4, or GLUL;
iv) HPGDS, PTGS2, ALOX5, OR AREG;
v) IL-33, IL-18, MSMB, SCGB1A1, SCGB3A1, PSCA, or LYPD2; or CCL26 and CCL11;
wherein an increase in the expression of any one of:
i) one or more of IL4, IL5, or IL13;
ii) Wnt;
iii) POSTN, PTHLH, ALOX15, SERPINB2, HS3ST1, CDH26, MMP10, CCL26;
iv) HPGDS, PTGS2, ALOX5, OR AREG; or
wherein a decrease in the expression of any one of:
i) Notch;
ii) SPINK5, ALDH3A1, CLCA4, or GLUL;
iii) MSMB, SCGB1A1, SCGB3A1, PSCA, or LYPD2, or
iv)CCL26 and CCL11; or
v) one or more markers from non-polyp cluster of Table 15.
is indicative of the type 2 inflammation induced tissue disease.

114. The method of claim 113, further comprising producing a single cell transcriptome/proteome for one or more cells from a second population of cells in a second sample to thereby provide transcriptome/proteome from the second population of cells.

115. The method of claim 114, wherein the transcriptome/proteome from the second population of cells is a reference sample, standard or a control.

116. The method of claim 115, wherein the tissue disease is nasal polyps.

117. The method of claim 115, wherein the first and second samples are from the same individual organism.

118. The method of claim 115 wherein the second sample is a reference sample, optionally from a second individual.

119. The method of claim 115, wherein t-distributed stochastic neighbor embedding is used to analyze the transcriptomes/proteome to determine sub-populations of cells.

120. A method of modulating basal cell proliferation, differentiation, maintenance and/or function comprising administering, to the basal cell, an effective amount of one or more modulating agents able to interact with an AP-1 transcription family member.

121. The method of claim 120, wherein the one or more modulating agent inhibits the AP-1 transcription family member, wherein inhibition of the AP-1 transcription family member induces differentiation of the basal cell.

122. The method of claim 121, wherein the AP-1 transcription family member is p63, FOXA1, or Bach2.

123. A method of determining cellular diversity in barrier tissue samples comprising;
detecting an amount of basal, fibroblast, myeloid, apical, glandular epithelium, differentiating/secretory, ciliated, plasma, endothelial, mast, and T cells in a sample, by detecting one more markers corresponding to each cell type as defined in claims 24 to 34.

124. The method of claim 123, further comprising detecting type 2 inflammation in in the sample by detecting a decrease in cellular diversity relative to a healthy barrier tissue reference.

125. The method of claim 124, wherein the decrease in cellular diversity comprises an increase in basal cells and/or a decrease in ciliated and glandular cells relative to the healthy barrier tissue reference.

126. A method of detecting type 2 inflammation in barrier tissues comprising;
detecting:
i) an increase in expression of one or more of DLK2, DLL1, JAG2, DKK3, POSTN, FN1, and TNC relative to a healthy barrier tissue reference;
ii) a decrease in SPINK5, ALDH3A1, CLCA4, an GLUL expression relative to a healthy barrier tissue reference;
iii) an increase in expression of one or more of JUN, FOXA1, BACH2, and p63 relative to a healthy barrier tissue reference; or
iv) a combination thereof.

127. A method of characterizing a cell phenotype, cell signature, cell expression profile, or cell expression program, the method comprising detecting one or more expression products from Table 1, Table 2, Table 3, Table 4, Table 5, Table 6, Table 7, Table 8, Table 9, Table 10, Table 11, Table 12, Table 13, Table 14, Table 15, or combination thereof.

128. The method according to any of the proceeding claims, wherein:
the cell phenotype, cell signature, cell expression profile, or cell expression program is microenvironment specific,
the cell phenotype, cell signature, cell expression profile, or cell expression program is found in a particular spatio-temporal context;
the cell phenotype, cell signature, cell expression profile, or cell expression program is specific to a particular pathological context;
a combination of cell subtypes having a particular cell phenotype, cell signature, cell expression profile, or cell expression program indicates a therapeutic or diagnostic outcome.
the cell phenotype, cell signature, cell expression profile, or cell expression program are used to deconvolute the network of cells present in a particular pathological condition.
the presence of specific cells and cell subtypes are indicative of a particular response to treatment, optionally including increased or decreased susceptibility to treatment, optional by a particular pharmaceutical agent or mode of therapy;
the cell phenotype, cell signature, cell expression profile, or cell expression program indicates the presence of one particular cell type.

129. A method for screening for drugs that induce or reduce a cell phenotype, cell signature, cell expression profile, or cell expression program, optionally in immune cells, the method comprising detecting changes in one or more expression products from Table 1, Table 2, Table 3, Table 4, Table 5, Table 6, Table 7, Table 8, Table 9, Table 10, Table 11, Table 12, Table 13, Table 14, Table 15, or combination thereof.

130. The method of claim 130, wherein the cell phenotype, cell signature, cell expression profile, or cell expression program is used for GE-HTS (Gene Expression-based High-Throughput Screening) and, optionally, a pharmacological screen is used to identify drugs that selectively activate barrier cells.
